(19) United States
(12) Patent Application Publication
(10) Pub. No.: US 2024/0124428 A1
(43) Pub. Date: Apr. 18, 2024

(54) HETEROCYCLIC INHIBITORS OF PCSK9

(71) Applicant: CARDIO THERAPEUTICS PTY LTD, Gordon (AU)

(72) Inventors: Phillip Albert COGHLAN, Gordon (AU); Alexandra Kalyna SUCHOWERSKA, Gordon (AU); James T. PALMER, Warrandyte (AU); James Anthony BONNAR, Gordon (AU); Benny EVISON, Gordon (AU)

(21) Appl. No.: 18/268,952

(22) PCT Filed: Dec. 21, 2021
(86) PCT No.: PCT/AU2021/051534

§ 371 Date: Jun. 21, 2023

(30) Foreign Application Priority Data
Dec. 21, 2020 (AU) 2020904774

(51) Int. Cl. C07D 405/14; A61K 45/06; A61P 9/10; C07D 401/10; C07D 401/14; C07D 403/10; C07D 403/14; C07D 413/14; C07D 417/14; C07D 471/04

(52) U.S. Cl. CPC C07D 405/14; A61K 45/06; A61P 9/10; C07D 401/10; C07D 401/14; C07D 403/10; C07D 403/14; C07D 413/14; C07D 417/14; C07D 471/04; C07B 2200/05

(57) ABSTRACT

This application relates to chemical compounds which may act as inhibitors of, or which may otherwise modulate the activity of, PCSK9, or a pharmaceutically acceptable salt, solvate, prodrug or polymorph thereof, and to compositions and formulations comprising such compounds, and methods of using and making such compounds. Compounds include compounds of Formula (I): (I) wherein R¹, R², R³ and L are described herein.

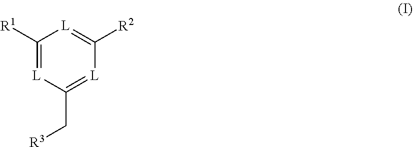

### CROSS REFERENCE TO RELATED APPLICATION

This application claims the benefit of Australian Provisional Application No. 2020904774 the entire contents of which are incorporated herein by reference.

### FIELD OF THE INVENTION

The present disclosure relates to compounds for the treatment of LDL related disorders, to their compositions and methods for their use, and to PCSK9 inhibition.

### BACKGROUND OF THE INVENTION

Cardiovascular diseases are said to cause an estimated 17.5 million (over 30%) of all deaths as of 2012 (E. Corey, The Pharmaceutical Journal, 2015). A particular risk factor, atherosclerosis, results from high levels of circulating low-density lipoprotein (LDL-C, a.k.a. “bad” cholesterol) in the blood. LDL-C accumulation in the inner walls of arteries results in atherosclerosis and can provoke an inflammatory response, which in turn can lead to cardiovascular events such as heart attack and stroke. Thus, LDL-C measurement is an effective surrogate marker for the risk of cardiovascular events.

Proprotein convertase subtilisin kexin type 9 (PCSK9) was discovered in 2003 (Seidah, N. G. et al, PNAS, 2003), is a serine protease, and is highly expressed in the liver. It is a genetically validated target for hypercholesterolemia (Abifadel, M. et al, Nature Genetics, 2003). Loss-of-function mutations of the PCSK9 gene have been linked to lower levels of LDL-C and a reduction of cardiovascular risk (Cohen, J. C. et al, NEJM, 2006). Its regulatory mechanisms have been reviewed (Lagace, T. A, Curr. Opin. Lipidol. (2014), 387-393). PCSK9 is synthesized as an enzyme precursor. Following synthesis, PCSK9 undergoes autocatalytic cleavage, which is required for secretion from the cell. The cleaved prodomain remains with PCSK9, blocking access to the active site of the enzyme. While LDL-C normally binds to the LDL receptor (LDL-R), which are together internalized and degraded intracellularly, PCSK9 attaches to the LDL-R/LDL complex for internalization/degradation. As a result, recirculation of LDL-R is reduced, resulting in increased circulatory LDL. Inhibition of PCSK9 or prevention of LDL-R attachment thereto results in increased cell surface expression of LDL-R, lowering circulatory LDL.

More recently, it was shown that PCSK9 regulation also has an effect on tumour growth. Inhibition of PCSK9 boosted the response of tumours to immune checkpoint therapy, through a mechanism that is independent of PCSK9's cholesterol-regulating functions (Liu et al. Nature, 2020). PCSK9's capacity to regulate cholesterol levels in the body lies in its ability to downregulate the cell-surface levels of low-density-lipoprotein. Based on the ability of PCSK9 to regulate a diverse group of cell-surface proteins, it was shown that Inhibiting PCSK9 increases the expression of major histocompatibility protein class I (MHC1) proteins on the tumour cell surface, promoting intratumoral infiltration of cytotoxic T cells and rendering tumours more responsive to immune checkpoint therapy.

Because PCSK9's only substrate is itself, targeting circulating PCSK9 by small molecule inhibitors is unlikely to represent an option for LDL reduction because the mechanism of action of PCSK9 in reducing cellular LDLR does not involve proteolytic activity. However, small cell-permeable molecules targeting the catalytic site of PCSK9 pro-enzyme could theoretically inhibit the auto-processing of PCSK9, thereby promoting its degradation in the ER. However, cross-reactivity associated with such inhibitors raises concern that PCSK9 pro-enzyme inhibition could co-inhibit other proprotein convertases. (Mousavi, S. A. et al., J. Int. Med. (2009) 266, 517-519).

Despite the discovery of PCSK9 and its role in LDL regulation, statins have served as the primary therapy used to prevent cardiovascular events. By inhibiting the rate-limiting enzyme HMG-CoA reductase, which has a vital role in internal (hepatic) cholesterol production through the reduction of 3-hydroxy-3-methylglutaryl coenzyme A to mevalonic acid, various statins can reduce LDL-C levels from 10-60% and have been shown to reduce the risk of heart attack and stroke.

Familial hypercholesterolemia (FH) is a hereditary disorder of LDL cholesterol metabolism, affects 1 in 250 persons and is characterized by greatly increased levels of LDL-C (Besseling, J. et al., J. Am. Coll. Cardiol. (2016) 68, 252-268). Patients with heterozygous FH are at 3- to 4-fold higher risk for coronary artery disease (CAD) and tend to develop CAD on average 10 years earlier in life than unaffected persons. Statins lower LDL cholesterol in patients with heterozygous FH, approximately to the same extent as in the general population while the average relative risk reduction of statins for CAD is estimated to be 22% per mmol/l among the general population it was unknown whether there is a comparable risk reduction in the setting of heterozygous FH because it would be unethical to withhold treatment from these patients. In the Besseling study to estimate the relative risk reduction for CAD and mortality by statins in heterozygous FH patients, the authors concluded that moderate- to high-intensity statin therapy lowered the risk for CAD and mortality by 44%. However, reduction in LDL-C is not considered sufficient in many cases. One mechanism by which statins display a countervailing mechanism is in the upregulation of sterol regulatory element binding protein 2 (SREBP-2, see Wong, J. et al., Biochem. J. (2006), 400, 485-491.). This increased activity results in the activation of both LDL receptors (LDLR) and PCSK9. Increased expression and secretion binds LDLR, resulting in higher LDLC. Thus, while statins reduce LDL via HMGCoA inhibition, their effect on SREPB acts as a counterbalance. Adding PCSK9 inhibitors to therapy can help override this mechanism.

While statins have been on the market for almost 30 years, some patients find statins to be ineffective or are burdened by intolerable side effects such as muscle pain (Abd, T. T., Jacobson, T. A., Expert Opinion on Drug Safety, p 373-387, 2011). Observationally, up to 10-15% of statin users develop muscle side effects ranging from mild myalgia to more severe symptoms. Furthermore, it has been reported that statin therapy is associated with a slightly higher risk of diabetes (2-17%, Sattar, N. et al., Lancet, (2010) 375, 735-742.) Given that familial hypercholesterolemia patients may not sufficiently benefit from statin therapy even in the absence of adverse side effects, there exists a need for alternative therapy avenues such as PCSK9 inhibition.

To date, there are no marketed small molecule inhibitors of PCSK9. Monoclonal antibody based drugs alirocumab and evolocumab have shown evidence of large improvements in lipid levels. These drugs are administered by injection, for instance biweekly. Alirocumab, when delivered every 2 weeks, showed greatest effect in heterozygous FH patients at cardiovascular risk who had not achieved LCL-C goals with statin therapy alone. Alirocumab also showed a moderate increase in “good” cholesterol (HDL-C) of 6-12% over this period. However, legal disputes over the intellectual property surrounding alirocumab have resulted in an injunction from its marketing in some jurisdictions. These issues, together with the substantially higher costs typically associated with monoclonal antibody production over small molecule inhibitors, clearly illustrates the very high need for competitive small molecule inhibitors of PCSK9.

Small molecule approaches have been described in the following: See WO2014170786, (Pfizer), WO2014150395, WO2014150326 (Shifa), WO2011051961, WO2014002106 (Cadila Healthcare) and US20120004223 (CVI), none of which have progressed beyond the discovery stage. Additional reported approaches include RNAi and gene-silencing oligonucleotides.

The present invention seeks to provide small molecule inhibitors of PCSK9.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

As discussed above, the present invention seeks to provide small molecule inhibitors of PCSK9.

In a first aspect, therefore, the invention provides a compound according to Formula (I):

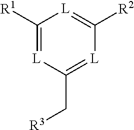

or a pharmaceutically acceptable salt, solvate, prodrug or polymorph thereof, wherein
L is CH or N;
R¹ is selected from the group consisting of —NHCOR¹¹ and

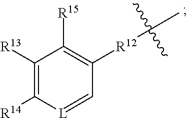

R² is selected from the group consisting of

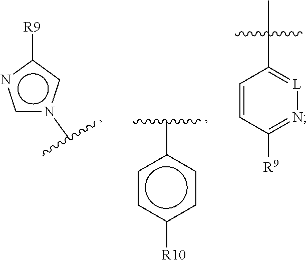

R³ is selected from the group consisting of —OCH₂CH₂NH₂,

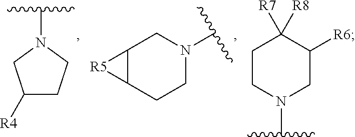

R⁴ is selected from the group consisting of —NH₂ and —NHCH₃;
R⁵ is selected from the group consisting of —NHCH₂CH₂O— and —OCH₂CH₂NH—;
R⁶ is selected from the group consisting of —NH₂, —OH, —NHCOR¹⁶, and —NHSO₂CH₂CH₃;
R⁷ is selected from the group consisting of —H, —OH, and —F;
R⁸ is selected from the group consisting of —H and —F;
R⁹ is selected from the group consisting of —CH₃ and —CF₃;
R¹⁰ is selected from the group consisting of —H and —CH₃;
R¹¹ is selected from the group consisting of imidazo[1,5-a]pyridin-1-yl,

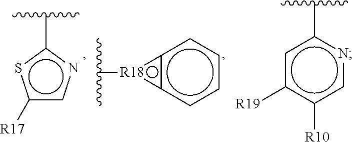

R¹² is selected from the group consisting of —CH₂NH—, —CONH—, —SO₂NH—,

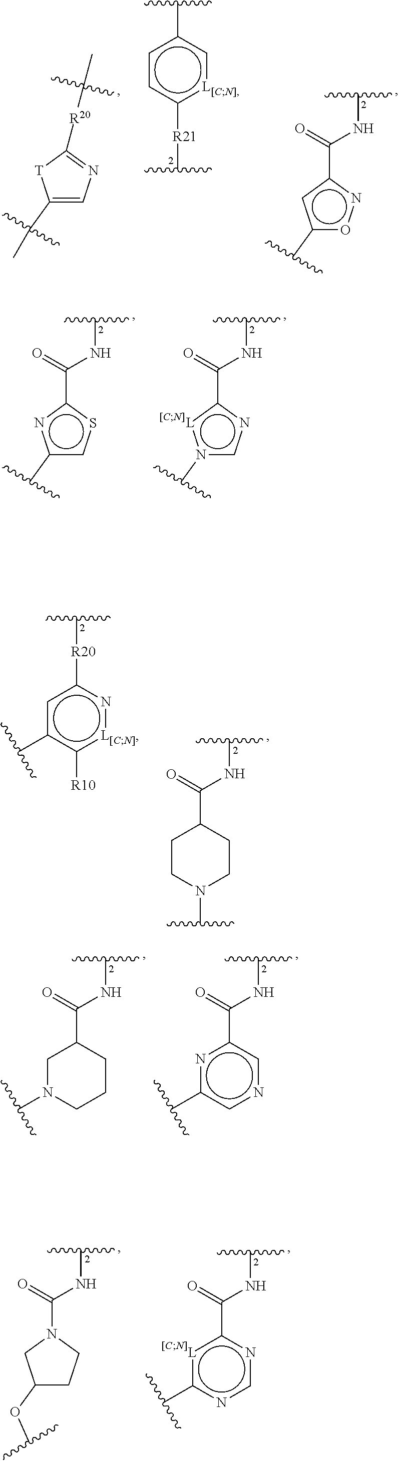

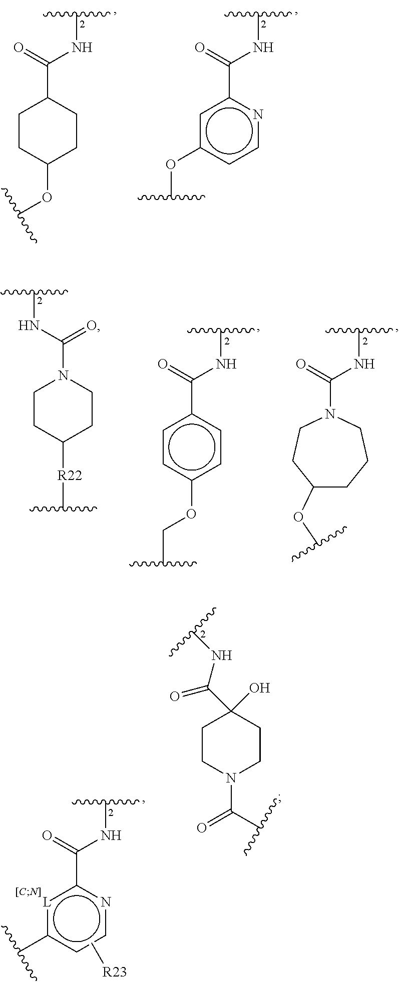

R¹³ is selected from the group consisting of —H, —CH₃, —F, —OCH₃, —CHCH₃OH, —SCH₃O₂, 2-methoxyethoxy, phenyl, oxolan-3-yloxy,

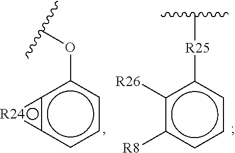

R¹⁴ is selected from the group consisting of —H, —CH₃, —OH, —F, —Cl, —OCH₃, cyclopropoxy, —OCF₃, —OCH(R¹⁰)CH₂R²⁷, and

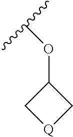

wherein Q is CH₂ or O;
R¹⁵ is selected from the group consisting of —H and —CHF₂;
R¹⁶ is selected from the group consisting of —CH₂CH₃, (3-hydroxyazetidin-1-yl)methyl, morpholin-4-yl, (pyridin-2-yl)methyl, 2-(1H-indol-3-yl)ethyl, and 2-{2,2-difluoro-10,12-dimethyl-1λ⁵,3-diaza-2-boratricyclo[7.3.0.0^{3,7}]dodeca-1(12),4,6,8,10-pentaen-1-ylium-2-uid-4-yl}ethyl;
R¹⁷ is selected from the group consisting of 1,3-thiazol-2-yl, cyclohexyl, morpholin-4-yl, 1,4-dioxan-2-yl, and 1,3-benzothiazol-5-yl;
R¹⁸ is selected from the group consisting of

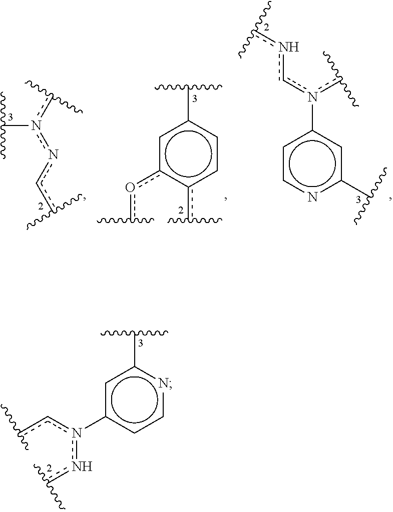

selected from the group consisting of —H, —C(CH₃)₃, 1,3-thiazol-4-yl, cyclohexyl, pyridin-2-yl, 4-methyl-1H-imidazol-1-yl, 4-methyl-1H-pyrazol-1-yl, 1,4-dioxan-2-yl, 3-azabicyclo[3.3.1]nonan-3-yl, 1,3-benzothiazol-5-yl, 2,3,4,5,6-pentamethylphenyl,

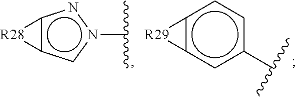

R²⁰ is selected from the group consisting of —CH₂NH— and —CONH—;
R²¹ is selected from the group consisting of —CONH— and

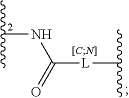

R²² is selected from the group consisting of —O— and —OCH₂—;
R²³ is selected from the group consisting of —CH₃ and —OCH₃;
R²⁴ is selected from the group consisting of

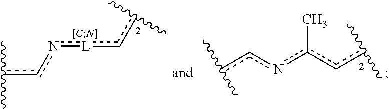

R²⁵ is selected from the group consisting of —CH₂O— and

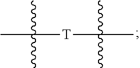

wherein T is O or S;
R²⁶ is selected from the group consisting of —H, —F, and —CHF₂;
R²⁷ is selected from the group consisting of —H, —OH, —OCH₃, and —OCH(R¹⁰)CH₂R³⁰;
R²⁸ is selected from the group consisting of

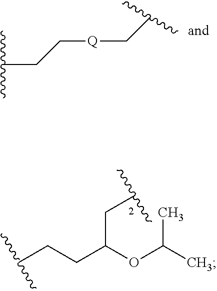

wherein Q is CH₂ or O;
R²⁹ is selected from the group consisting of —OCH₂O— and —OCH₂CH₂O—;
R³⁰ is selected from the group consisting of —H and —OCH₂CH₂OCH₂CH₂OCH₂CH₂OCH₂CH₂OCH₂CH₂OR¹⁰.

In a second aspect, therefore, the invention provides a compound according to Formula (II):

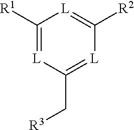

or a pharmaceutically acceptable salt, solvate, prodrug or polymorph thereof,
wherein
L is CH or N;
R¹ is selected from the group consisting of —NHCOR¹¹ and

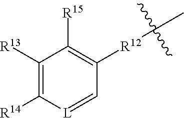

R² is selected from the group consisting of

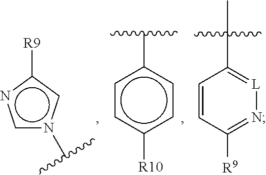

R³ is selected from the group consisting of —OCH₂CH₂NH₂,

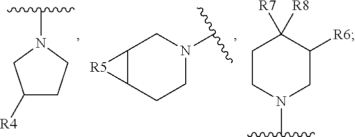

R⁴ is selected from the group consisting of —NH₂ and —NHCH₃;
R⁵ is selected from the group consisting of —NHCH₂CH₂O— and —OCH₂CH₂NH—;
R⁶ is selected from the group consisting of —NH₂, —OH, —NHCOR¹⁶, and —NHSO₂CH₂CH₃;
R⁷ is selected from the group consisting of —H, —OH, and —F;
R⁸ is selected from the group consisting of —H and —F;
R⁹ is selected from the group consisting of —CH₃ and —CF₃;
R¹⁰ is selected from the group consisting of —H and —CH₃;
R¹¹ is selected from the group consisting of imidazo[1,5-a]pyridin-1-yl,

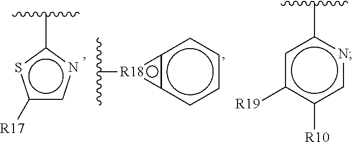

R¹² is selected from the group consisting of —CONH—,

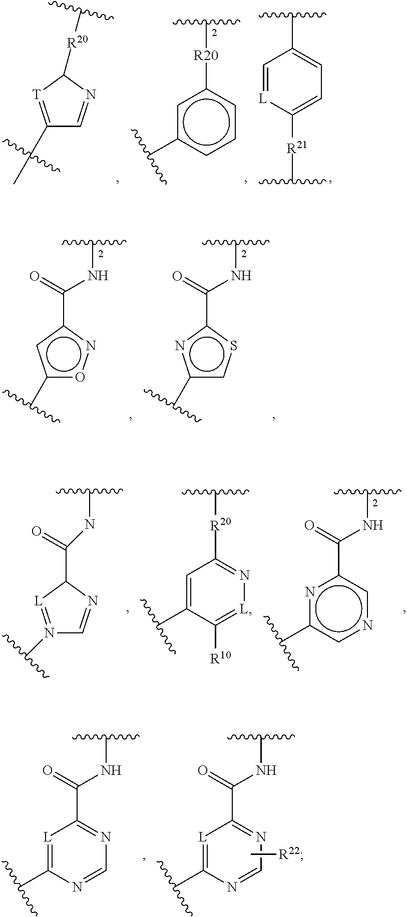

wherein T is O or S and wherein L is as previously defined
R¹³ is selected from the group consisting of —H, —CH₃, —OCH₃, —CHCH₃OH, —SCH₃O₂, and 2-methoxyethoxy;
R¹⁴ is selected from the group consisting of —H, —CH₃, —OH, —F, —OCH₃, cyclopropoxy, —OCH(R¹⁰)CH₂R²³, and

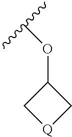

Q wherein Q is CH₂ or O;
R¹⁵ is selected from the group consisting of —H and —CHF₂;
R¹⁶ is selected from the group consisting of —CH₂CH₃, (3-hydroxyazetidin-1-yl)methyl, morpholin-4-yl, (pyridin-2-yl)methyl, 2-(1H-indol-3-yl)ethyl, and 2-{2,2-difluoro-10,12-dimethyl-1λ⁵,3-diaza-2-boratricyclo[7.3.0.0^{3,7}]dodeca-1(12),4,6,8,10-pentaen-1-ylium-2-uid-4-yl}ethyl;
R¹⁷ is selected from the group consisting of 1,3-thiazol-2-yl, cyclohexyl, morpholin-4-yl, 1,4-dioxan-2-yl, and 1,3-benzothiazol-5-yl;
R¹⁸ is selected from the group consisting of

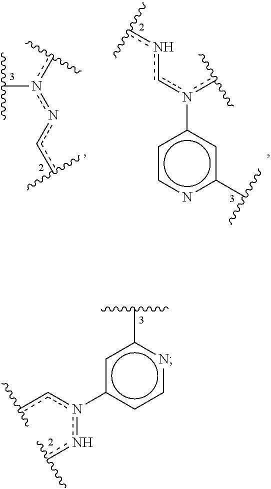

R¹⁹ is selected from the group consisting of —H, —C(CH₃)₃, 1,3-thiazol-4-yl, cyclohexyl, pyridin-2-yl, 4-methyl-1H-imidazol-1-yl, 4-methyl-1H-pyrazol-1-yl, 1,4-dioxan-2-yl, 3-azabicyclo[3.3.1]nonan-3-yl, 1,3-benzothiazol-5-yl, 2,3,4,5,6-pentamethylphenyl,

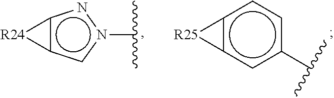

R²⁰ is selected from the group consisting of —CH₂NH— and —CONH—;
R²¹ is selected from the group consisting of —CONH— and

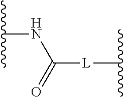

wherein L is as previously defined;
R²² is selected from the group consisting of —CH₃ and —OCH₃;
R²³ is selected from the group consisting of —H, —OH, —OCH₃, and —OCH(R¹⁰)CH₂R²⁶;
R²⁴ is selected from the group consisting of

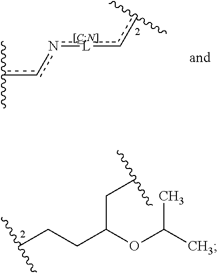

R²⁵ is selected from the group consisting of —OCH₂O— and —OCH₂CH₂O—;
R²⁶ is selected from the group consisting of —H and —OCH₂CH₂OCH₂CH₂OCH₂CH₂OCH₂CH₂OCH₂CH₂OR¹⁰.
   There are some numbers indicated on the structures of some chemical moieties. These are an artefact of the program Markush Editor, used to generate the Markush structure and are not intended to have any significance.

In a third aspect of the disclosure there is provided a compound according to Formula (I):

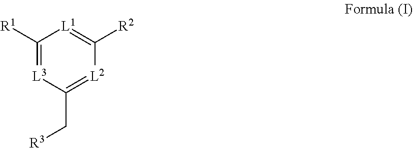

or a pharmaceutically acceptable salt, solvate, prodrug or polymorph thereof,
wherein
L¹, L² and L³ are independently CH or N;
R¹ is —NHCOR¹¹ or

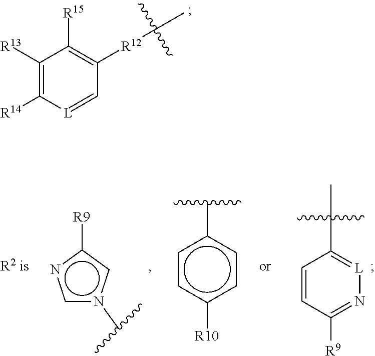

wherein L is CH or N;
R³ is —OCH₂CH₂NH₂,

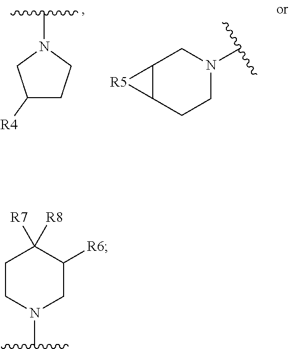

R⁴ is —NH₂ or —NHCH₃;
R⁵ is —NHCH₂CH₂O— or —OCH₂CH₂NH—;
R⁶ is —NH₂, —OH, —NHCOR¹⁶, or —NHSO₂CH₂CH₃;
R⁷ is —H, —OH, or —F;
R⁸ is —H or —F;
R⁹ is —CH₃ or —CF;
R¹⁰ is —H or —CH₃;
R¹¹ is

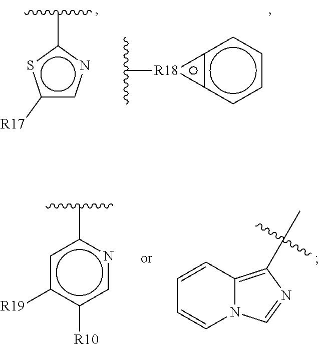

R¹² is —CH₂NH*—, —CONH*—, —SO₂NH*—,

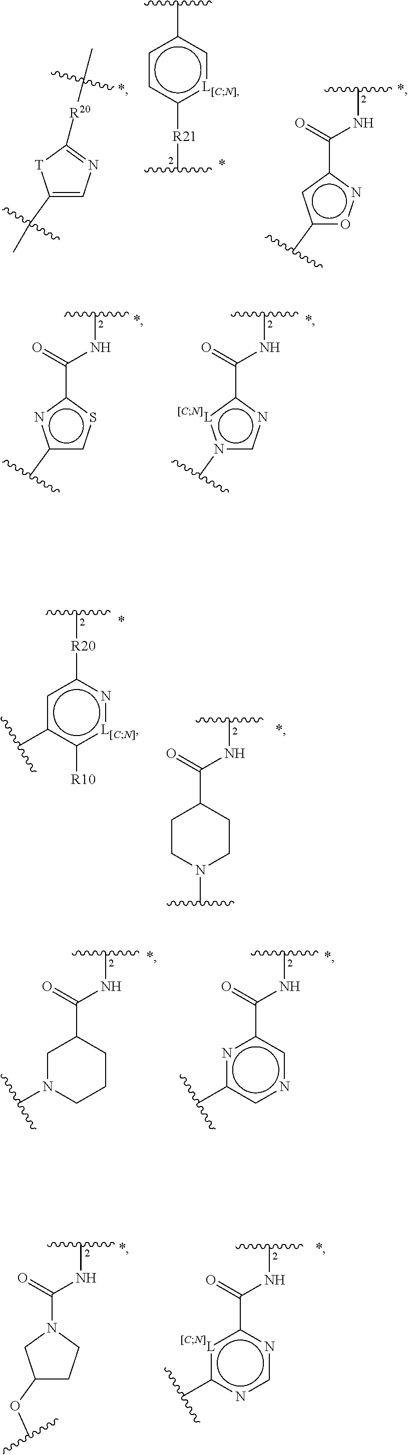

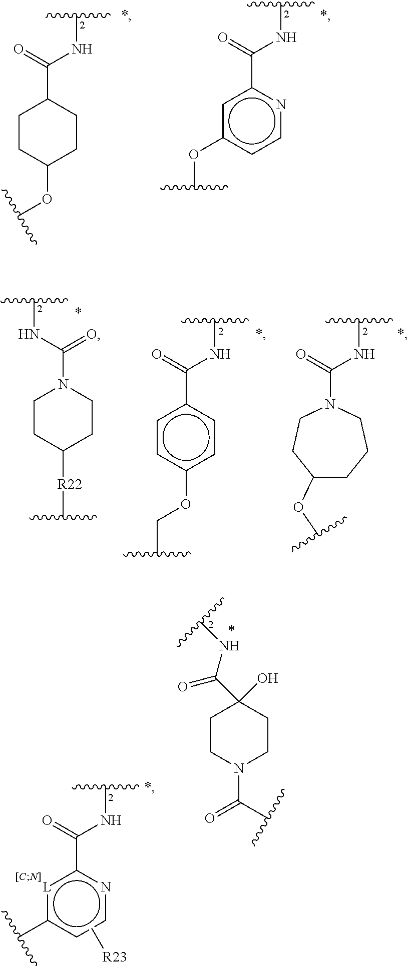

wherein the * indicates the attachment of R¹² to the carbon of the central ring of formula (I) and the other attachment point signifies the attachment of R¹² to the phenyl ring of R¹; wherein L is CH or N;
R¹³ is —H, —CH₃, —F, —OCH₃, —CHCH₃OH, —SCH₃O₂, 2-methoxyethoxy, phenyl or oxolan-3-yloxy;
R¹⁴ is selected from the group consisting of —H, —CH₃, —OH, —F, —Cl, —OCH₃, cyclopropoxy, —OCF₃—OCH(R¹⁰)CH₂R²⁷ or

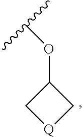

wherein Q is CH₂ or O;
R¹⁵ is —H or —CHF₂;
R¹⁶ is —CH₂CH₃,

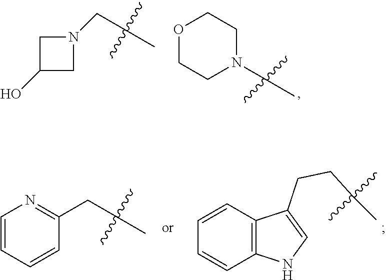 [SMILES: *CN1CC(O)C1 ; *N1CCOCC1 ; *Cc1ccccn1 ; *CCc1c[nH]c2ccccc12]

R¹⁷ is

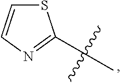 [SMILES: *c1nccs1]

cyclohexyl,

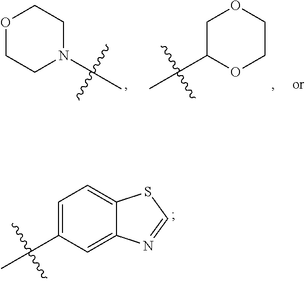 [SMILES: *N1CCOCC1 ; *C1COCCO1 ; *c1ccc2scnc2c1]

R¹⁸ is selected from the group consisting of

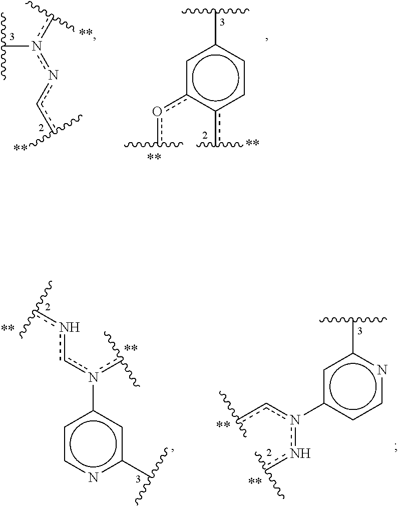

wherein the ** indicates the attachment points of R¹⁸ which form the fused ring and the remaining attachment point indicates the attachment to the carbonyl carbon of R¹; R¹⁹ is —H, —C(CH₃)₃,

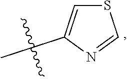 [SMILES: *c1cscn1]

cyclohexyl,

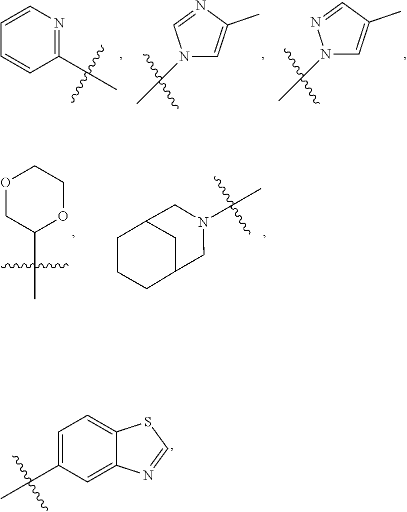

2,3,4,5,6-deuterated phenyl,

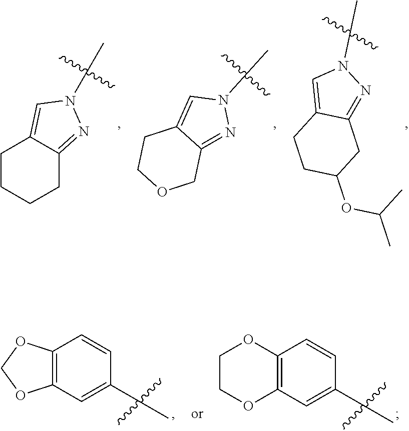 [SMILES: *n1cc2c(n1)CCCC2 ; *n1cc2c(n1)COCC2 ; *n1cc2c(n1)CC(OC(C)C)CC2 ; *c1ccc2c(c1)OCO2 ; *c1ccc2c(c1)OCCO2]

R²⁰ is —CH₂NH*— or —CONH*—; wherein the * indicates the attachment of R²⁰ to the carbon of the central ring of Formula (I);
R²¹ is —CONH*—,

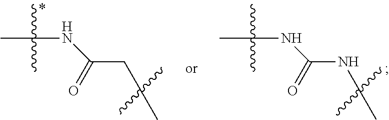 [SMILES: *CC(=O)N* ; *NC(=O)N*]

wherein the * indicates the preferred attachment of R²¹ to the carbon of the central ring of Formula (I);
R²² is —O— or —*OCH₂—; wherein the * indicates the attachment of R²² to the phenyl ring of R¹²;
R²³ is —CH₃ or —OCH₃;
R²⁶ is —H, —F, or —CHF₂;
R²⁷ is —H, —OH, —OCH₃, or —OCH(R¹⁰)CH₂R³⁰;
R³⁰ is —H or —OCH₂CH₂OCH₂CH₂OCH₂CH₂OCH₂CH₂OCH₂CH₂OR¹⁰, wherein R¹⁰ is H or CH₃.

In an embodiment of the third aspect there is provided a compound or a pharmaceutically acceptable salt, solvate, prodrug or polymorph thereof wherein:
R¹² is —CONH*—,

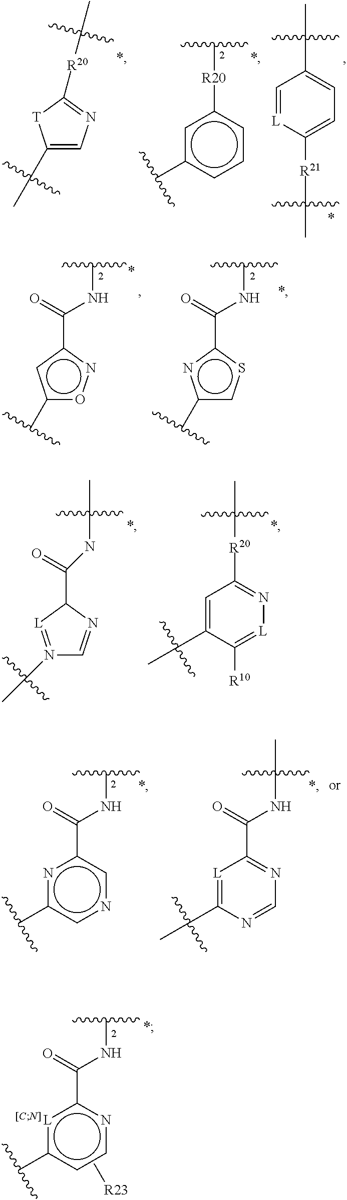

wherein T is O or S, wherein L is CH or N, and wherein the * indicates the attachment of R¹² to the carbon on the central ring of Formula (I);
R¹³ is —H, —CH₃, —OCH₃, —CHCH₃OH, —SCH₃O₂, or 2-methoxyethoxy;
R¹⁴ is —H, —CH₃, —OH, —F, —OCH₃, cyclopropoxy, —OCH₂CH₂R²⁷, —OCH(CH₃)CH₂R²⁷

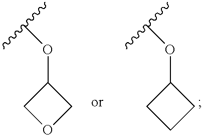

wherein R²⁷ is as defined herein.
R¹⁸ is

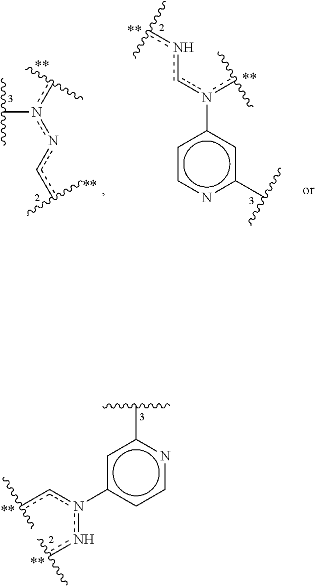

wherein the ** indicates the attachment points of R¹⁸ which form the fused ring and the remaining attachment point indicates the attachment to the carbonyl carbon of R¹; and
R²³ is —CH₃ or —OCH₃.

In an embodiment of the third aspect there is provided a compound or a pharmaceutically acceptable salt, solvate, prodrug or polymorph or deuterated analogue thereof wherein the compound is a compound of Formula (III)

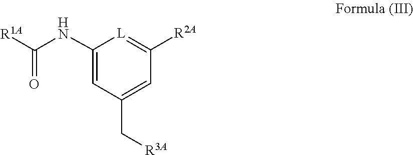

wherein
L is CH or N;
R^{1A} is

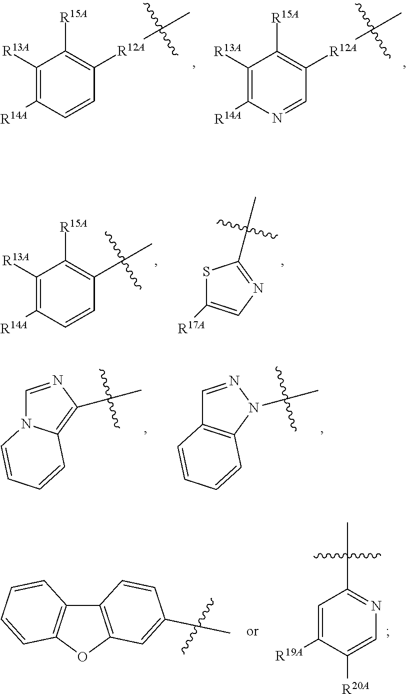

wherein R^{12A} is

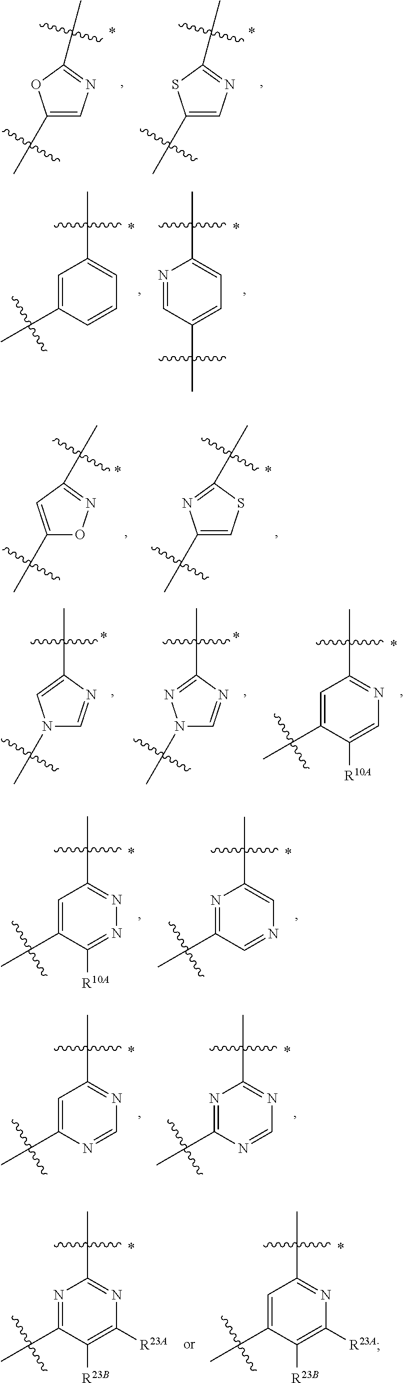

wherein the * signifies the attachment of R^{12A} to the carbonyl carbon of Formula (III) and the other attachment point signifies the attachment of R^{12A} to the phenyl ring of R^{1A};
R^{2A} and R^{3A} are as defined for R² and R³ in in the third aspect;
R^{10A} is —H or —CH₃;
R^{23A} and R^{23B} are independently —H, —CH₃ or —OCH₃;
R^{13A} is as defined for R¹³ in in the third aspect;
R^{14A} is —H, —CH₃, —OH, —F, —OCH₃, cyclopropoxy, —OCH₂CH₂OCH₃, —OCH₂CH₂OH, —OCH(CH₃)₂, —OCH₂CH₂OCH(CH₃)₂, —O(CH₂CH₂O)₇CH₃, —O(CH₂CH₂O)₇H,

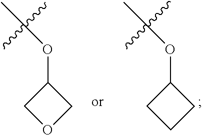 [SMILES: *OC1COC1 ; *OC1CCC1]

R^{15A} is as defined for R¹⁵ in the third aspect;
R^{17A} is as defined for R¹⁷ in the third aspect;
R^{19A} is as defined for R¹⁹ in the third aspect; and
R^{20A} is —H or —CH₃.

In an embodiment of the second aspect there is provided a compound or a pharmaceutically acceptable salt, solvate, prodrug or polymorph or deuterated analogue thereof, wherein the compound is a compound of formula (IIIa) or formula (IIIb)

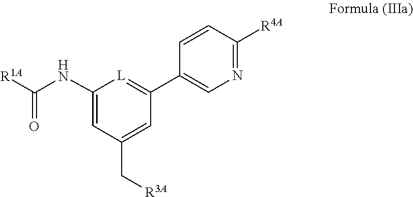

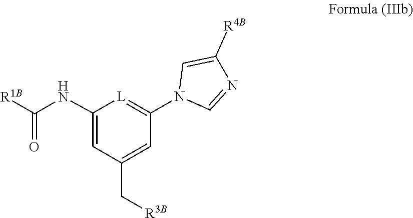

wherein L is CH or N;
wherein R^{4A} and R^{4B} are independently CH₃ or CF₃;
R^{1A} and R^{1B} are independently as defined for R^{1A} in the third aspect; and
R^{3A} and R^{3B} are independently as defined for R^{3A} in the third aspect.

In an embodiment of the third aspect there is provided a compound or a pharmaceutically acceptable salt, solvate, prodrug or polymorph or deuterated analogue thereof, wherein the compound is a compound of formula (IVa) or formula (IVb)

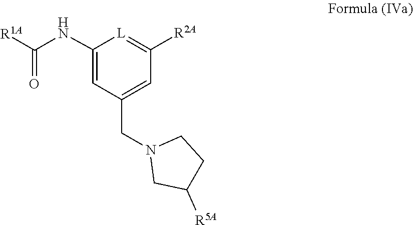

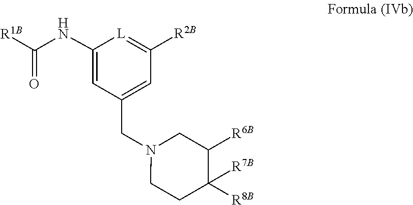

wherein L is CH or N;
wherein R^{5A} is —NH₂ or —NHCH₃;
R^{6B} is defined as for R⁶ in the third aspect;
R^{7B} is defined as for R⁷ in the third aspect
R^{8B} is defined as for R⁷ in the third aspect;
R^{1A} and R^{1B} are independently as defined for R^{1A} in the third aspect; and
R^{2A} and R^{2B} are independently as defined for R^{2A} in the third aspect.

In a fourth aspect there is provided a compound or a pharmaceutically acceptable salt, solvate, prodrug or polymorph or deuterated analogue thereof wherein the compound is a compound of Formula (V)

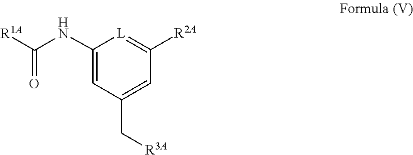

wherein L is CH or N;
wherein R^{1A} is

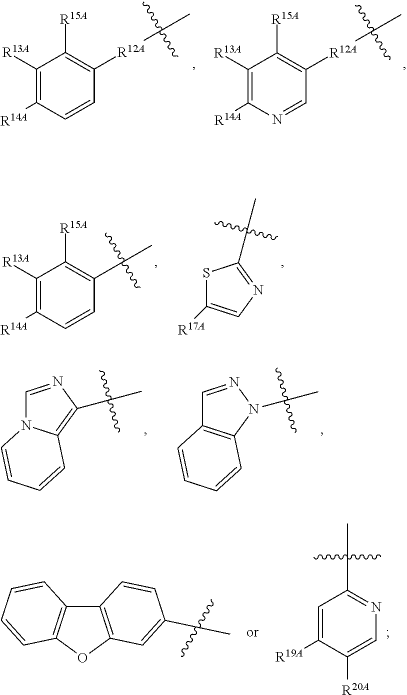

wherein R^{12A} is selected from

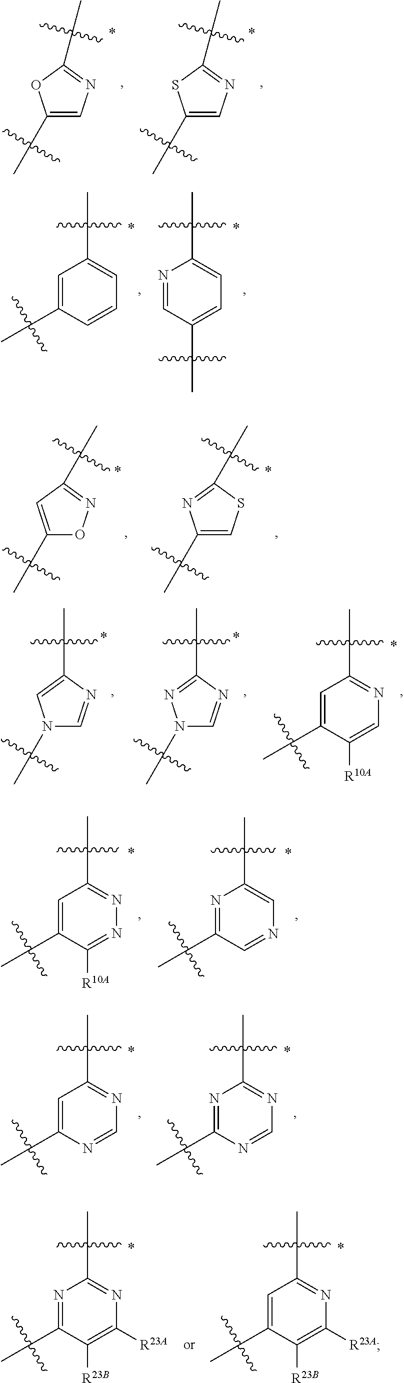

wherein the * signifies the attachment of R^{12A} to the carbonyl carbon of Formula (V) and the other attachment point signifies the attachment of R^{12A} to the phenyl ring of R^{1A};
R^{10A} is —H, —CH₃C₂-C₆alkyl, or C₁-C₆ fluoroalkyl;
R^{23A} and R^{23B} are independently —H, —CH₃, —OCH₃, C₂-C₆alkyl, or C₂-C₆alkoxy;
R^{13A} is —H, —CH₃, —F, —OCH₃, —CH₂CH₃OH, —SCH₃O₂, 2-methoxyethoxy, phenyl, or oxolan-3-yloxy;
R^{14A} is —H, —CH₃, C₂-C₆ alkyl, —OH, —F, —Cl, —I, —Br, —OCH₃, —OC₂-C₆alkyl, cyclopropoxy, —OCH₂CH₂OCH₃, —OCH₂CH₂OH, —OCH(CH₃)₂, —OCH₂CH₂OCH(CH₃)₂, —O(CH₂CH₂O)₇CH₃, —O(CH₂CH₂O)₇H, —O(CH2CH2O)ₙCH₃, or —O(CH2CH3O)ₙH,
wherein n is 2, 3, 4, 5, or 6.

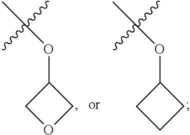 [SMILES: *OC1COC1 ; *OC1CCC1]

R^{15A} is —H, —CHF₂, —CH₂F, —CF₃, —C₁-C₆alkyl, or C₂-C₆ fluoroalkyl;
R^{17A} is

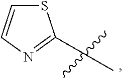 [SMILES: *c1nccs1]

cyclohexyl, C₃-C₅ cycloalkyl,

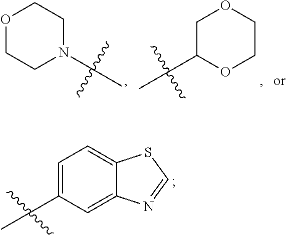 [SMILES: *N1CCOCC1 ; *C1COCCO1 ; *c1ccc2scnc2c1]

R^{19A} is —H, —C(CH₃)₃, C₁-C₃alkyl, C₅-C₆alkyl, C₁-C₆ fluoroalkyl,

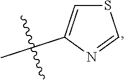 [SMILES: *c1cscn1]

cyclohexyl, C₃-C₅ cycloalkyl,

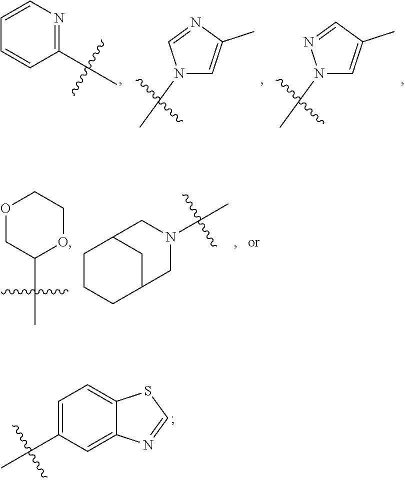

R^{20A} is —H, —CH₃, C₂-C₆ alkyl, C₁-C₆ fluoroalkyl, substituted C₁ alkyl, or substituted C₁-C₆ alkyl;
R^{2A} is selected from the group consisting of

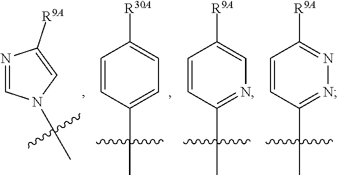

wherein R^{9A} is —CH₃, —CF₃, C₂-C₆alkyl, C₂-C₆fluoroalkyl, —CH₂F, —CHF₂;
R^{30A} is —H, —F, —CH₃, —C₂-C₆alkyl, or C₁-C₆ fluoroalkyl;
R^{3A} is selected from the group consisting of —OCH₂CH₂NH₂,

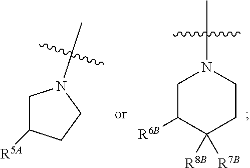

wherein R^{5A} is —NH₂, —NHCH₃ or —C₂-C₆alkylamino;
R^{6B} is —NH₂, —NH₂CH₃, C₂-C₆alkylamino, —OH, —NHCOR^{16B}, or —NHSO₂CH₂CH₃;
R^{16B} is —CH₂CH₃, —CH₃, —C₃-C₆alkyl,

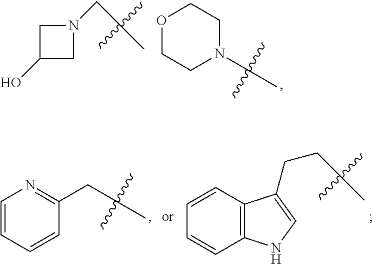

R^{7B} is —H, —OH, —F, —Cl, —Br, —I or —C₁-C₃ alkyl; and
R^{8B} is —H, —F, —Cl, —Br, —I or —C₁-C₃ alkyl.

In an embodiment of the fourth aspect there is provided a compound or a pharmaceutically acceptable salt, solvate, prodrug or polymorph or deuterated analogue thereof wherein the compound is of formula (Va) or formula (Vb):

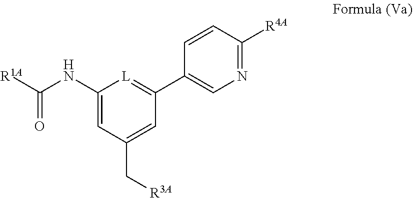

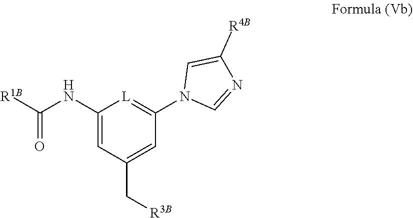

wherein
L is CH or N;
R^{4A} and R^{4B} are independently —CH₃, —CF₃, —C₂-C₆ alkyl or —C₂-C₆ fluoroalkyl;
R^{1A} and R^{1B} are independently as defined for R^{1A} as previously defined; and
R^{3A} and R^{3B} are independently as defined for R^{3A} as previously defined.

In an embodiment of the fourth aspect there is provided a compound or a pharmaceutically acceptable salt, solvate, prodrug or polymorph or deuterated analogue thereof wherein the compound is a compound of formula (VIa) or formula (VIb):

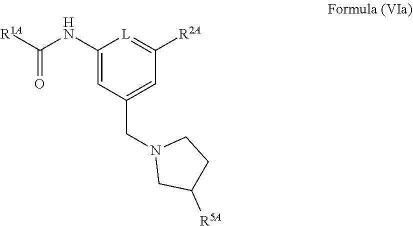

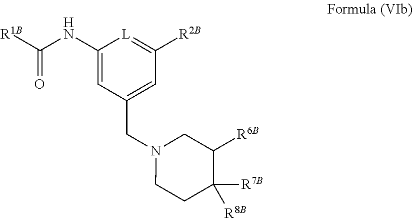

wherein
L is CH or N;
R^{5A} is —NH₂, —NHCH₃ or —C₂-C₆alkylamino;
R^{6B} is —NH₂, —NH₂CH₃, —C₂-C₆alkylamino;
R^{7B} is —H, —OH, —F, —Cl, —Br, or —I;
R^{8B} is —H, —F, —Cl, —Br, or —I;
R^{1A} and R^{1B} are independently as defined for R^{1A} as previously defined; and
R^{3A} and R^{3B} are independently as defined for R^{3A} as previously defined.

In one aspect, there is provided a composition comprising a compound according to Formula (I) and/or Formula (II), or a pharmaceutically acceptable salt, solvate, prodrug or polymorph thereof, and a pharmaceutically acceptable excipient.

In another aspect, there is provided a composition comprising a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph thereof, and a pharmaceutically acceptable excipient.

In one aspect, there is provided a method for inhibiting PCSK9 in a subject in need thereof, the method comprising administering a therapeutically effective amount of a compound or a pharmaceutically acceptable salt, solvate, prodrug or polymorph thereof of Formula (I) and/or Formula (II) to a subject.

In another aspect, there is provided a method for inhibiting PCSK9 in a subject in need thereof the method comprising administering a therapeutically effective amount of a compound or a pharmaceutically acceptable salt, solvate, deuterated analogue, prodrug or polymorph thereof of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) to a subject.

In one aspect, there is provided a method for reducing LDL in a subject in need thereof, the method comprising administering a therapeutically effective amount of a compound or a pharmaceutically acceptable salt, solvate, prodrug or polymorph thereof of Formula (I) and/or Formula (II) to a subject.

In another aspect, there is provided a method for reducing LDL in a subject in need thereof, the method comprising administering a therapeutically effective amount of a compound or a pharmaceutically acceptable salt, solvate, deuterated analogue, prodrug or polymorph thereof of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) to a subject.

In one aspect, there is provided a method for treating a disease or condition responsive to PCSK9 inhibition in a subject in need thereof, wherein the disease or condition is any one of the following: cardiovascular disease, cerebrovascular disease, atherosclerosis and/or their associated diseases or their symptoms, the method comprising administering a therapeutically effective amount of a compound according to Formula (I) and/or Formula (II) or a pharmaceutically acceptable salt, solvate, prodrug or polymorph thereof to a subject.

In another aspect, there is provided a method for treating a disease or condition responsive to PCSK9 inhibition in a subject in need thereof, wherein the disease or condition is any one of the following: cardiovascular disease, cerebrovascular disease, atherosclerosis and/or their associated diseases or their symptoms, the method comprising administering a therapeutically effective amount of a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph thereof to a subject.

In each of the abovementioned methods of treatment, a composition of the invention may alternatively be administered. That is, a composition comprising a therapeutically effective amount of a compound according Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph may be administered.

In another aspect, there is provided use of a compound of Formula (I) and/or Formula (II) or a pharmaceutically acceptable salt, solvate, prodrug or polymorph thereof, in the preparation of a medicament for the inhibition of PCSK9 in a subject.

In another aspect, there is provided use of a compound of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph thereof, in the preparation of a medicament for the inhibition of PCSK9 in a subject.

In another aspect, there is provided use of a compound of Formula (I) and/or Formula (II) or a pharmaceutically acceptable salt, solvate, prodrug or polymorph thereof, in the preparation of a medicament for the reduction of LDL in a subject.

In another aspect, there is provided use of a compound of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, or a composition thereof, in the preparation of a medicament for the reduction of LDL in a subject.

In another aspect, there is provided use of a compound of Formula (I) and/or Formula (II) or a pharmaceutically acceptable salt, solvate, prodrug or polymorph thereof, or a composition thereof, in the preparation of a medicament for the treatment of a disease or condition in a subject, wherein the disease or condition is any one of the following: cardiovascular disease, cerebrovascular disease, atherosclerosis and/or their associated diseases or their symptoms.

In another aspect, there is provided use of a compound Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph thereof in the preparation of a medicament for the treatment of a disease or condition in a subject, wherein the disease or condition is any one of the following: cardiovascular disease, cerebrovascular disease, atherosclerosis and/or their associated diseases or their symptoms.

In each of the abovementioned uses, a composition of the invention may alternatively be used in the preparation of a medicament. That is, a composition comprising a therapeutically effective amount of a compound according Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph.

In another aspect, there is provided use of a compound according to Formula (I) and/or Formula (II) or a pharmaceutically acceptable salt, solvate, prodrug or polymorph thereof, for the inhibition of PCSK9.

In another aspect, there is provided use of a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph thereof, for the inhibition of PCSK9.

In another aspect, there is provided use of a compound according to Formula (I) and/or Formula (II) or a pharmaceutically acceptable salt, solvate, prodrug or polymorph thereof, for the reduction of LDL.

In another aspect, there is provided use of a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph thereof, for the reduction of LDL.

In another aspect, there is provided use of a compound of Formula (I) and/or Formula (II) or a pharmaceutically acceptable salt, solvate, prodrug or polymorph thereof, for the treatment of a disease or condition in a subject, wherein the disease or condition is any one of the following: cardiovascular disease, cerebrovascular disease, atherosclerosis and/or their associated diseases or their symptoms.

In another aspect, there is provided use of a compound of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph thereof, for the treatment of a disease or condition in a subject, wherein the disease or condition is any one of the following: cardiovascular disease, cerebrovascular disease, atherosclerosis and/or their associated diseases or their symptoms.

In each of the abovementioned uses, a composition of the invention may alternatively be used. That is, a composition comprising a therapeutically effective amount of a compound according Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph may be used.

In yet another aspect, there is provided a compound according to Formula (I) and/or Formula (II) or a pharmaceutically acceptable salt, solvate, prodrug or polymorph thereof, for use in inhibiting PCSK9.

In yet another aspect, there is provided a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph thereof, for use in inhibiting PCSK9.

In another aspect, there is provided a compound according to Formula (I) and/or Formula (II) or a pharmaceutically acceptable salt, solvate, prodrug or polymorph thereof, for use in reducing LDL.

In another aspect, there is provided a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph thereof, for use in reducing LDL.

In another aspect, there is provided a compound according to Formula (I) and/or Formula (II) or a pharmaceutically acceptable salt, solvate, prodrug or polymorph thereof, for use in the treatment of a disease or condition responsive to PCSK9 inhibition in a subject, wherein the disease or condition is any one of the following: cardiovascular disease, cerebrovascular disease, atherosclerosis and/or their associated diseases or their symptoms.

In another aspect, there is provided a compound according Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph thereof, for use in the treatment of a disease or condition responsive to PCSK9 inhibition in a subject, wherein the disease or condition is any one of the following: cardiovascular disease, cerebrovascular disease, atherosclerosis and/or their associated diseases or their symptoms.

In each of the abovementioned uses, it may be the composition of the invention for use. That is, a composition comprising a therapeutically effective amount of a compound according Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph.

In yet another aspect, there is provided a compound according to Formula (I) and/or Formula (II) or a pharmaceutically acceptable salt, solvate, prodrug or polymorph thereof, when used for inhibiting PCSK9.

In yet another aspect, there is provided a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph thereof, when used for inhibiting PCSK9.

In yet another aspect, there is provided a compound according to Formula (I) and/or Formula (II) or a pharmaceutically acceptable salt, solvate, prodrug or polymorph thereof, when used for reducing LDL.

In yet another aspect, there is provided a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph thereof, when used for reducing LDL.

In yet another aspect, there is provided a compound of Formula (I) and/or, Formula (II) or a pharmaceutically acceptable salt, solvate, prodrug or polymorph thereof, when used for the treatment of a disease or condition in a subject, wherein the disease or condition is any one of the following: cardiovascular disease, cerebrovascular disease, atherosclerosis and/or their associated diseases or their symptoms.

In yet another aspect, there is provided a compound of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph thereof, when used for the treatment of a disease or condition in a subject, wherein the disease or condition is any one of the following: cardiovascular disease, cerebrovascular disease, atherosclerosis and/or their associated diseases or their symptoms.

In each of the abovementioned uses, it may be the composition of the invention when used. That is, a composition comprising a therapeutically effective amount of a compound according Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, deuterated analogue, prodrug or polymorph.

Any embodiment herein shall be taken to apply mutatis mutandis to any other embodiment unless specifically stated otherwise.

The present disclosure is not to be limited in scope by the specific embodiments described herein, which are intended for the purpose of exemplification only. Functionally-equivalent products, compositions and methods are clearly within the scope of the invention, as described herein.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Mechanism of LDL uptake following PCSK9-LDLR binding.

FIG. 2: a) Sequence alignment of sequences for existing PCSK9 structures and key species from NCBI database; b) lack of sequence conservation across the PCSK family (PCSK1 to PCSK7 and PCSK9); The sequences and alignments in the Figures are based on a particular UNIPROT sequence database.

FIG. 3: Chemical structures and IC₅₀ (μM) for values as determined by an in vitro PCSK9-LDLR binding assay. The hashtag symbol denotes the compounds that exhibited a linear dose-response. The * symbol indicates that the depth of the IC50 curve is shallow, perhaps due to limited solubility of the compound. A $ symbol indicates that the compound was assayed twice and therefore duplicate IC50 values are shown.

FIG. 4A: In vitro ADME and in vivo cassette data. The efflux ratios and pharmacokinetics (PK) properties of the compounds. The compounds were assessed for their oral suitability in an in vitro assay utilizing the human colon epithelial cancer cell line Caco-2. The permeability coefficient (Papp) denotes the permeability of the drug through a monolayer of cells. The PK parameters of half-life (T_{1/2}) and area under the curve (AUC) of the compounds were assessed following a single intravenous (IV) injection (0.4 mg/kg) in mice (male, C57BL/6).

FIG. 4B: Tabulating hERG (% inhibition), HepG2 cell toxicity, and CYP (% inhibition) data for a selection of compounds. FIG. 4C demonstrates the cytotoxicity profiles of compounds 29, 122 and 131 in HepG2 cells following 48 h of exposure to the compounds.

FIG. 5A-C: The mean plasma concentration-time profiles from dedicated pharmacokinetic analyses of compounds 29, 122 and 131. Mean plasma concentration-time profiles of compound 29 (FIG. 5A), compound 122 (FIG. 5B) and compound 131 (FIG. 5C) after a PO dose of 50 mg/kg in female C57BL/6 mice (N=3/time point).

FIG. 6: A) The percentage decrease in LDLR expression levels on the surface of human primary lymphocytes upon the addition of recombinant PCSK9-D374Y to cells relative to non-PCSK9-D374Y-treated controls in the presence of either 0, 1 or 4 μM test and control compounds. Alirocumab was used as a positive control for compound 3f, compound 29 and alirocumab. B)-E) graphically illustrate the results summarized in FIG. 6A.

FIG. 7. The effect of treatment with compound 29 following 30 mg/kg and 50 mg/kg bid oral dosing on plasma total cholesterol levels, normalised to control.

FIG. 8. The effect of treatment with compound 29 following 30 mg/kg and 50 mg/kg bid oral dosing on plasma PCSK9 protein levels.

FIG. 9. The effect of treatment with compound 29 following 30 mg/kg and 50 mg/kg bid oral dosing on cholesterol levels fractionated by lipoprotein type at 28 days post-dosing.

FIG. 10. Plasma total cholesterol levels (FIG. 10A) and plasma non-HDL-cholesterol levels (FIG. 10B) of female APOE*3-Leiden.CETP mice treated orally with either vehicle (solid circles), 4.9 mg/kg/day atorvastatin (solid triangles), 50 mg/kg compound 29 twice per day (solid squares) or a combination of 4.9 mg/kg/day atorvastatin and 50 mg/kg compound 29 twice per day (solid diamonds).

FIG. 11. The fractionation of cholesterol levels according to lipoprotein type in pooled plasma samples of female APOE*3-Leiden.CETP mice following 35 days of treatment. The mice were treated orally with either vehicle (solid circles), 4.9 mg/kg/day atorvastatin (solid triangles), 50 mg/kg compound 29 twice per day (solid squares) or a combination of 4.9 mg/kg/day atorvastatin and 50 mg/kg compound 29 twice per day (solid diamonds).

FIG. 12. Plasma total PCSK9 levels of female APOE*3-Leiden.CETP mice treated orally with either vehicle (solid circles), 4.9 mg/kg/day atorvastatin (solid triangles), 50 mg/kg compound 29 twice per day (solid squares) or a combination of 4.9 mg/kg/day atorvastatin and 50 mg/kg compound 29 twice per day (solid diamonds).

FIG. 13. The hepatic levels of the LDL receptor in APOE*3-Leiden.CETP mice treated orally with either vehicle, 4.9 mg/kg/day atorvastatin, 50 mg/kg compound 29 twice per day or a combination of 4.9 mg/kg/day atorvastatin and 50 mg/kg compound 29 twice per day as indicated.

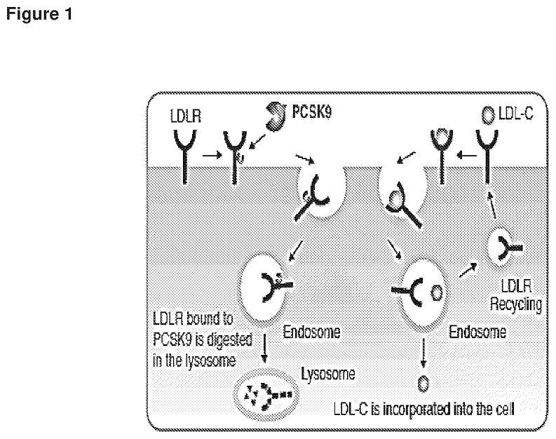

### DETAILED DESCRIPTION OF THE EMBODIMENTS

### Compounds of the Invention

In one aspect, therefore, the invention provides a compound according to Formula (I):

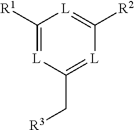

or a pharmaceutically acceptable salt, solvate, prodrug or polymorph thereof,
wherein L is CH or N;
R¹ is selected from the group consisting of —NHCOR¹¹ and

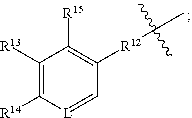

R² is selected from the group consisting of

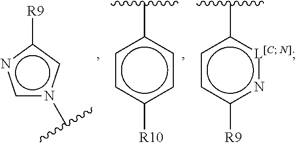

R³ is selected from the group consisting of —OCH₂CH₂NH₂,

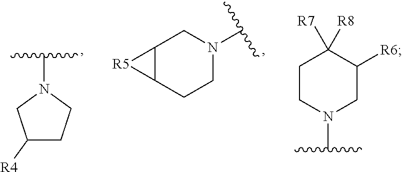

R⁴ is selected from the group consisting of —NH₂ and —NHCH₃;
R⁵ is selected from the group consisting of —NHCH₂CH₂O— and —OCH₂CH₂NH—;
R⁶ is selected from the group consisting of —NH₂, —OH, —NHCOR¹⁶, and NHSO₂CH₂CH₃;
R⁷ is selected from the group consisting of —H, —OH, and —F;
R⁸ is selected from the group consisting of —H and —F;
R⁹ is selected from the group consisting of —CH₃ and —CF₃;
R¹⁰ is selected from the group consisting of —H and —CH₃;
R¹¹ is selected from the group consisting of imidazo[1,5-a]pyridin-1-yl,

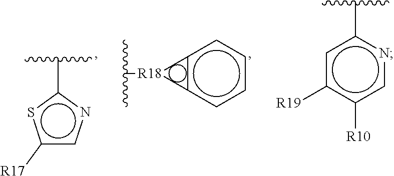

R¹² is selected from the group consisting of —CH₂NH—, —CONH—, —SO₂NH—,

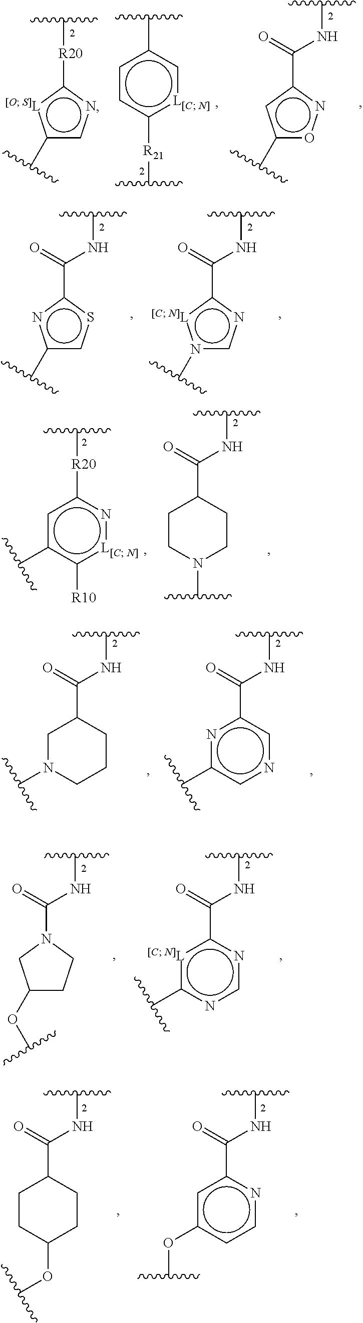

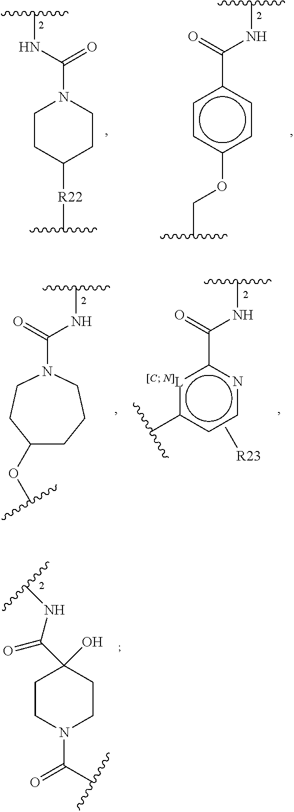

R¹³ is selected from the group consisting of —H, —CH₃, —F, —OCH₃, —CHCH₃OH, —SCH₃O₂, 2-methoxyethoxy, phenyl, oxolan-3-yloxy,

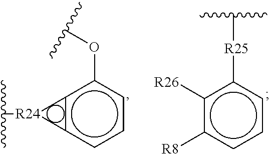

R¹⁴ is selected from the group consisting of —H, —CH₃, —OH, —F, —Cl, —OCH₃, cyclopropoxy, —OCF₃, —OCH(R¹⁰)CH₂R²⁷, and

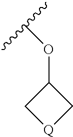

Q wherein Q is CH₂ or O;
R¹⁵ is selected from the group consisting of —H and —CHF₂;
R¹⁶ is selected from the group consisting of —CH₂CH₃, (3-hydroxyazetidin-1-yl)methyl, morpholin-4-yl, (pyridin-2-yl)methyl, 2-(1H-indol-3-yl)ethyl, and 2-{2,2-difluoro-10,12-dimethyl-1λ⁵,3-diaza-2-boratricyclo[7.3.0.0^{3,7}]dodeca-1(12),4,6,8,10-pentaen-1-ylium-2-uid-4-yl}ethyl;
R¹⁷ is selected from the group consisting of 1,3-thiazol-2-yl, cyclohexyl, morpholin-4-yl, 1,4-dioxan-2-yl, and 1,3-benzothiazol-5-yl;
R¹⁸ is selected from the group consisting of

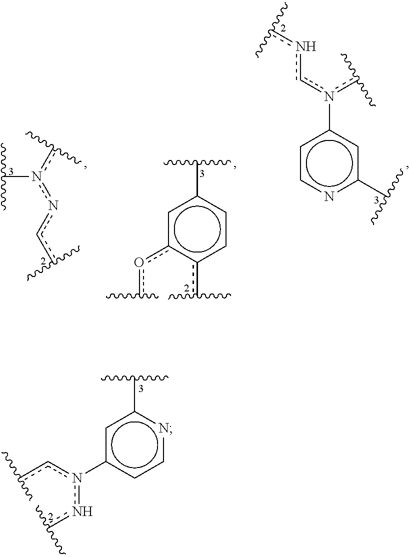

R¹⁹ is selected from the group consisting of —H, —C(CH₃)₃, 1,3-thiazol-4-yl, cyclohexyl, pyridin-2-yl, 4-methyl-1H-imidazol-1-yl, 4-methyl-1H-pyrazol-1-yl, 1,4-dioxan-2-yl, 3-azabicyclo[3.3.1]nonan-3-yl, 1,3-benzothiazol-5-yl, 2,3,4,5,6-pentamethylphenyl,

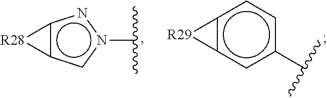

R²⁰ is selected from the group consisting of —CH₂NH— and —CONH—;
R²¹ is selected from the group consisting of —CONH— and

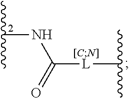

R²² is selected from the group consisting of —O— and —OCH₂—;
R²³ is selected from the group consisting of —CH₃ and —OCH₃;
R²⁴ is selected from the group consisting of

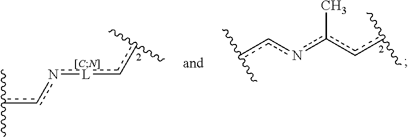

R²⁵ is selected from the group consisting of —CH₂O— and

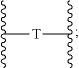

wherein T is O or S;
R²⁶ is selected from the group consisting of —H, —F, and —CHF₂;
R²⁷ is selected from the group consisting of —H, —OH, —OCH₃, and —OCH(R¹⁰)CH₂R³⁰;
R²⁸ is selected from the group consisting of

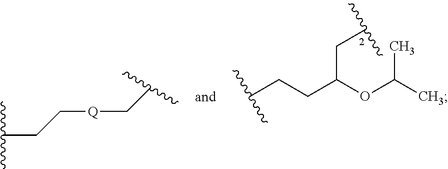

wherein Q is CH₂ or O;
R²⁹ is selected from the group consisting of —OCH₂O— and —OCH₂CH₂O—;
R³⁰ is selected from the group consisting of —H and —OCH₂CH₂OCH₂CH₂OCH₂CH₂OCH₂CH₂OCH₂CH₂OR¹⁰.

In another aspect, therefore, the invention provides a compound according to Formula (II):

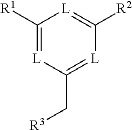

or a pharmaceutically acceptable salt, solvate, prodrug or polymorph thereof,
wherein
L is CH or N;
R¹ is selected from the group consisting of —NHCOR¹¹ and

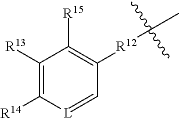

R² is selected from the group consisting of

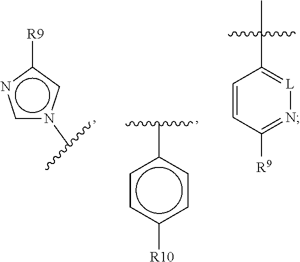

R³ is selected from the group consisting of —OCH₂CH₂NH₂,

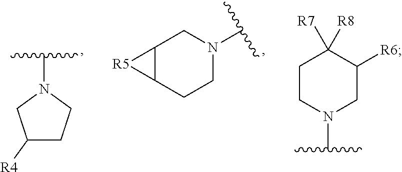

R⁴ is selected from the group consisting of —NH₂ and —NHCH₃;
R⁵ is selected from the group consisting of —NHCH₂CH₂O— and —OCH₂CH₂NH—;
R⁶ is selected from the group consisting of —NH₂, —OH, —NHCOR¹⁶, and —NHSO₂CH₂CH₃;
R⁷ is selected from the group consisting of —H, —OH, and —F;
R⁸ is selected from the group consisting of —H and —F;
R⁹ is selected from the group consisting of —CH₃ and —CF₃;
R¹⁰ is selected from the group consisting of —H and —CH₃;
R¹¹ is selected from the group consisting of imidazo[1,5-a]pyridin-1-yl,

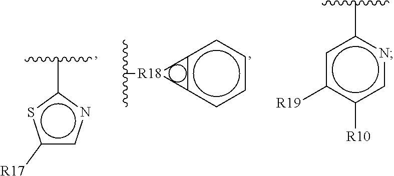

R¹² is selected from the group consisting of —CONH—,

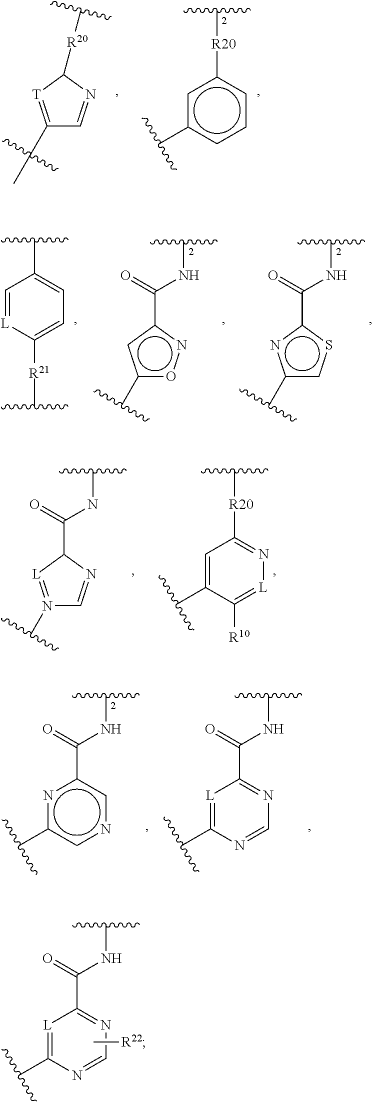

wherein T is O or S and wherein L is as previously defined
R¹³ is selected from the group consisting of —H, —CH₃, —OCH₃, —CHCH₃OH, —SCH₃O₂, and 2-methoxyethoxy;
R¹⁴ is selected from the group consisting of —H, —CH₃, —OH, —F, —OCH₃, cyclopropoxy, —OCH(R¹⁰)CH₂R²³, and

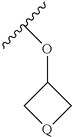

wherein Q is CH₂ or O;
R¹⁵ is selected from the group consisting of —H and —CHF₂;
R¹⁶ is selected from the group consisting of —CH₂CH₃, (3-hydroxyazetidin-1-yl)methyl, morpholin-4-yl, (pyridin-2-yl)methyl, 2-(1H-indol-3-yl)ethyl, and 2-{2,2-difluoro-10,12-dimethyl-1λ⁵,3-diaza-2-boratricyclo[7.3.0.0^{3,7}]dodeca-1(12),4,6,8,10-pentaen-1-ylium-2-uid-4-yl}ethyl;
R¹⁷ is selected from the group consisting of 1,3-thiazol-2-yl, cyclohexyl, morpholin-4-yl, 1,4-dioxan-2-yl, and 1,3-benzothiazol-5-yl;
R¹⁸ is selected from the group consisting of

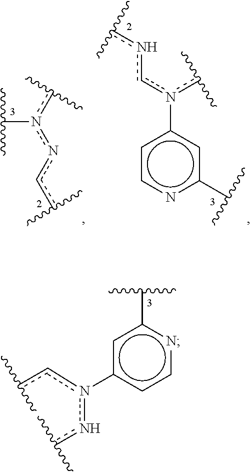

R¹⁹ is selected from the group consisting of —H, —C(CH₃)₃, 1,3-thiazol-4-yl, cyclohexyl, pyridin-2-yl, 4-methyl-1H-imidazol-1-yl, 4-methyl-1H-pyrazol-1-yl, 1,4-dioxan-2-yl, 3-azabicyclo[3.3.1]nonan-3-yl, 1,3-benzothiazol-5-yl, 2,3,4,5,6-pentamethylphenyl,

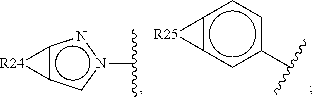

R²⁰ is selected from the group consisting of —CH₂NH— and —CONH—;
R²¹ is selected from the group consisting of —CONH— and

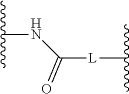

wherein L is as previously defined;
R²² is selected from the group consisting of —CH₃ and —OCH₃;
R²³ is selected from the group consisting of —H, —OH, —OCH₃, and —OCH(R¹⁰)CH₂R²⁶;
R²⁴ is selected from the group consisting of

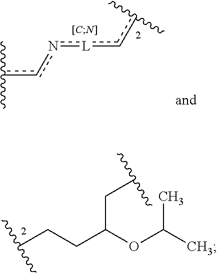

R²⁵ is selected from the group consisting of —OCH₂O— and —OCH₂CH₂O—;
R²⁶ is selected from the group consisting of —H and —OCH₂CH₂OCH₂CH₂OCH₂CH₂OCH₂CH₂OCH₂CH₂OR¹⁰.

Individual Substituent Selection

In embodiments of third aspect of the disclosure there is provided a compound according to Formula (I):

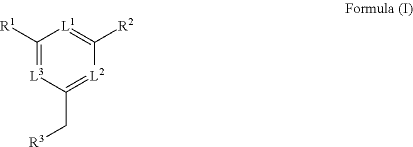

or a pharmaceutically acceptable salt, solvate, prodrug or polymorph thereof,
wherein
L¹, L² and L³ are independently CH or N; preferably not more than one of L¹, L² or L³ is N, for example is some embodiments L¹ is N or CH, and L² and L³ are CH, or L² is N or CH and L¹ and L³ are CH or L³ is N or CH and L¹ and L² are CH; more preferably L¹ is CH or N and L² and L³ are CH; even more preferably L¹ and L² and L³ are CH.
R¹ is —NHCOR¹¹ or

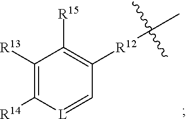

wherein L is CH or N;
R² is

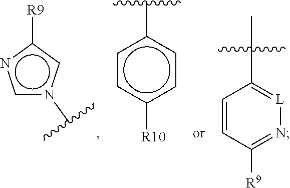

R³ is —OCH₂CH₂NH₂,

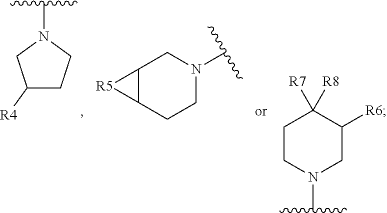

R⁴ is —NH₂ or —NHCH₃;
R⁵ is —NHCH₂CH₂O— or —OCH₂CH₂NH—;
R⁶ is —NH₂, —OH, —NHCOR¹⁶, or —NHSO₂CH₂CH₃;
R⁷ is —H, —OH, or —F;
R⁸ is —H or —F;
R⁹ is —CH₃ or —CF₃;
R¹⁰ is —H or —CH₃;
R¹¹ is

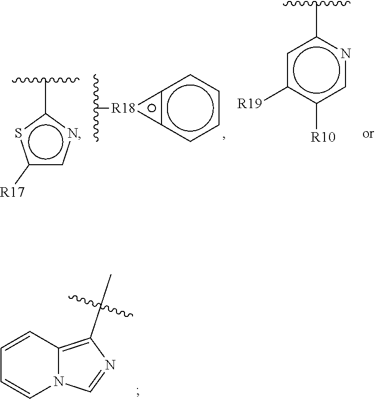

R¹² is —CH₂NH*—, —CONH*—, —SO₂NH*—,

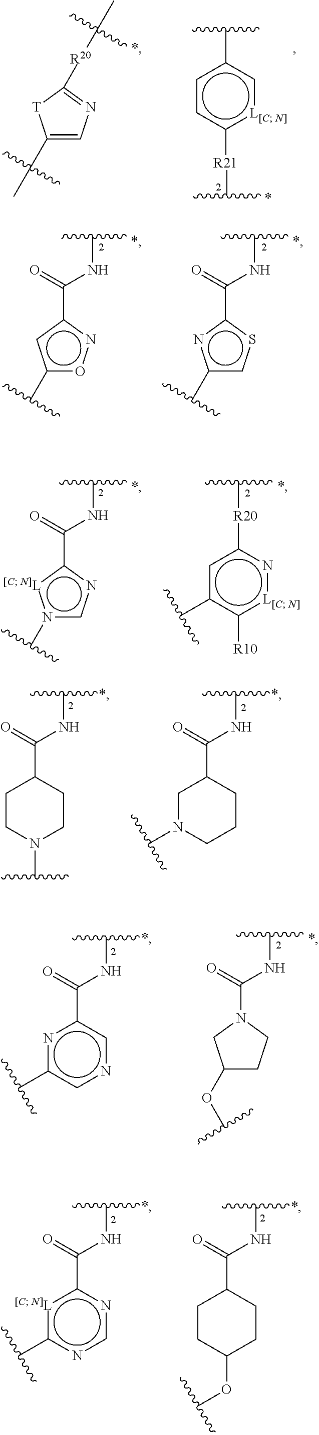

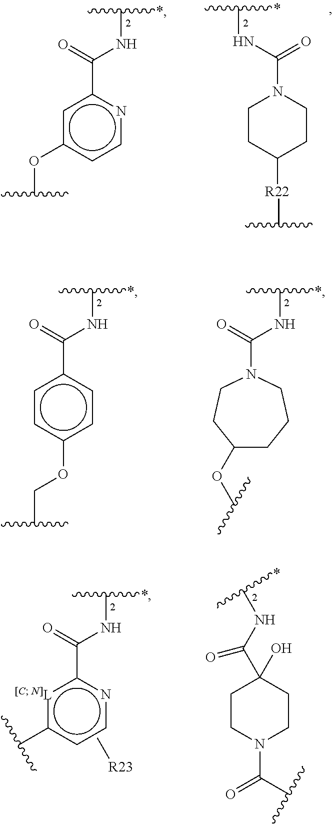

wherein the * indicates the attachment of R¹² to the carbon of the central ring of formula (I) and the other attachment point signifies the attachment of R¹² to the phenyl ring of R¹, and wherein T is O or S;
R¹³ is —H, —CH₃, —F, —OCH₃, —CHCH₃OH, —SCH₃O₂, 2-methoxyethoxy, phenyl or oxolan-3-yloxy;
R¹⁴ is selected from the group consisting of —H, —CH₃, —OH, —F, —Cl, —OCH₃, cyclopropoxy, —OCF₃—OCH(R¹⁰)CH₂R²⁷ or

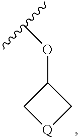

wherein Q is CH₂ or O;
R¹⁵ is —H or —CHF₂;
R¹⁶ is —CH₂CH₃,

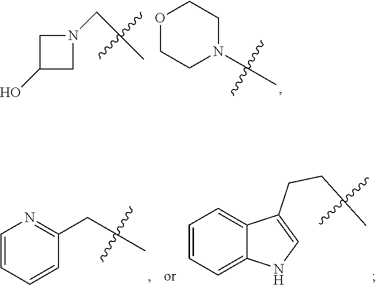

R¹⁷ is

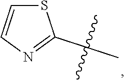 [SMILES: *c1nccs1]

cyclohexyl,

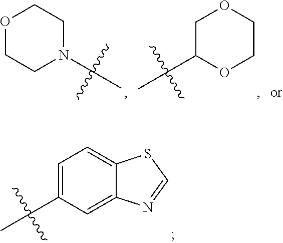 [SMILES: *N1CCOCC1 ; *C1COCCO1 ; *c1ccc2scnc2c1]

R¹⁸ is selected from the group consisting of

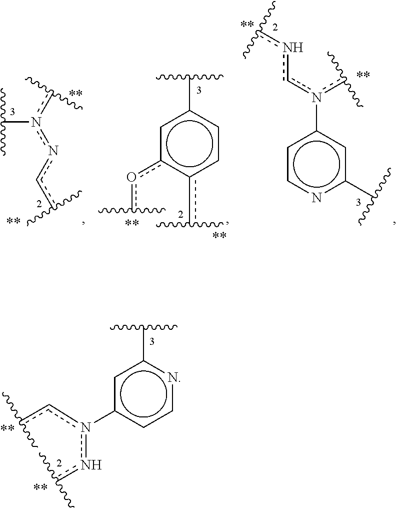

wherein the ** indicates the attachment points of R¹⁸ which form the fused ring and the remaining attachment point indicates the attachment to the carbonyl carbon of R¹; and wherein the dashed lines indicate that R18 when attached forms an aromatic ring structure.
R¹⁹ is —H, —C(CH₃)₃,

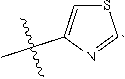 [SMILES: *c1cscn1]

cyclohexyl,

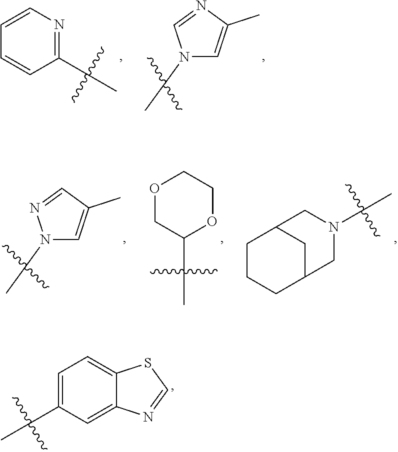

2,3,4,5,6-deuterated phenyl,

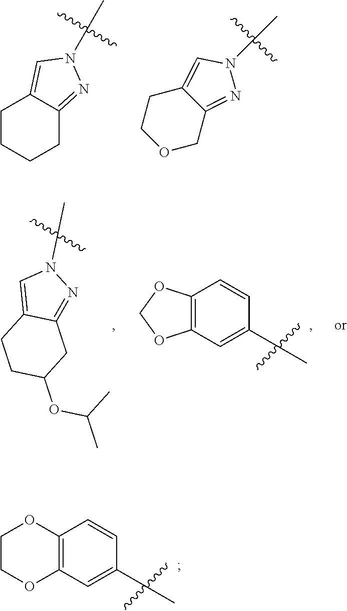 [SMILES: *n1cc2c(n1)CCCC2 ; *n1cc2c(n1)COCC2 ; *n1cc2c(n1)CC(OC(C)C)CC2 ; *c1ccc2c(c1)OCO2 ; *c1ccc2c(c1)OCCO2]

R²⁰ is —CH₂NH*— or —CONH*—; wherein the * indicates the attachment of R²⁰ to the carbon of the central ring of Formula (I);
R²¹ is —CONH*—,

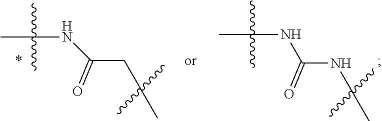 [SMILES: *CC(=O)N* ; *NC(=O)N*]

wherein the * indicates the preferred attachment of R²¹ to the carbon of the central ring of Formula (I);
R²² is —O— or —*OCH₂—; wherein the * indicates the attachment of R²² to the phenyl ring of R¹²;
R²³ is —CH₃ or —OCH₃;
R²⁶ is —H, —F, or —CHF₂;
R²⁷ is —H, —OH, —OCH₃, or —OCH(R¹⁰)CH₂R³⁰;
R³⁰ is —H or —OCH₂CH₂OCH₂CH₂OCH₂CH₂OCH₂CH₂OCH₂CH₂OR¹⁰, wherein R¹⁰ is H or CH₃.

In a preferred embodiment of the third aspect R¹² is —CONH*—,

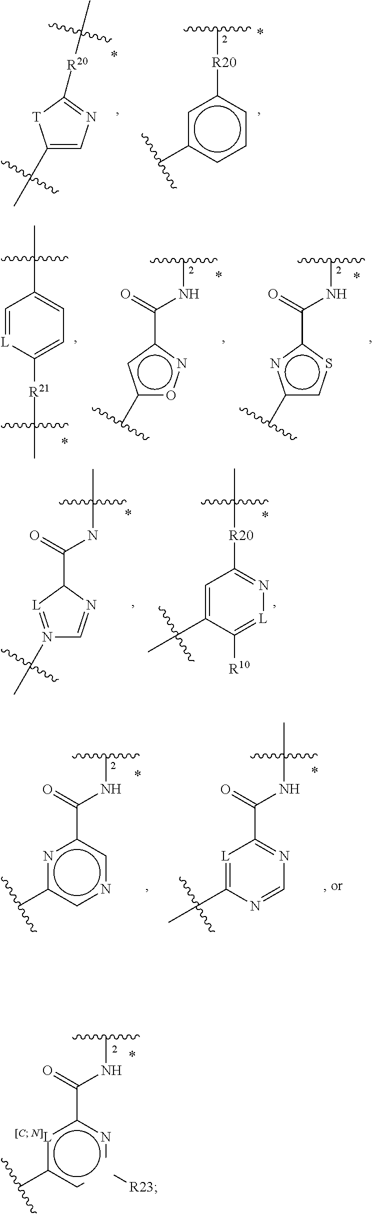

wherein T is O or S, wherein L is CH or N, and wherein the * indicates the attachment of R¹² to the carbon on the central ring of Formula (I).

In a preferred embodiment of the third aspect R¹³ is —H, —CH₃, —OCH₃, —CHCH₃OH, —SCH₃O₂, or 2-methoxyethoxy.

In a preferred embodiment of the third aspect R¹⁴ is —H, —CH₃, —OH, —F, —OCH₃, cyclopropoxy, —OCH₂CH₂R²⁷, —OCH(CH₃)CH₂R²⁷

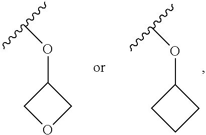 [SMILES: *OC1COC1 ; *OC1CCC1]

In a preferred embodiment of the third aspect R¹⁸ is

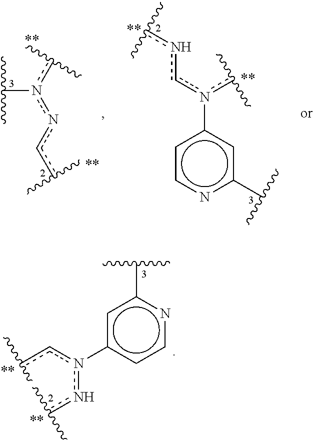

In a preferred embodiment of the third aspect R²³ is —CH₃ or —OCH₃.

In a preferred embodiment of the third aspect there is provided a compound or a pharmaceutically acceptable salt, solvate, prodrug or polymorph or deuterated analogue thereof wherein the compound is a compound of formula (III)

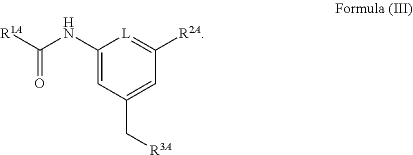

In preferred embodiments L is CH or N; in some embodiments L is more preferably CH; in some embodiments L is more preferably N.

In preferred embodiments R^{1A} is

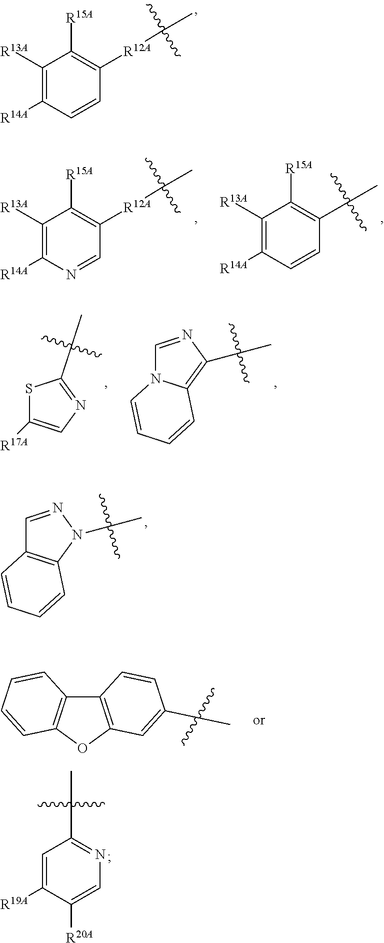

more preferably R^{1A} is

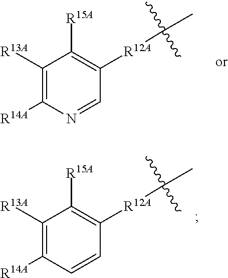

even more preferably R^{1A} is

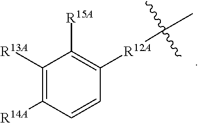

In a preferred embodiment of the third aspect R^{12A} is

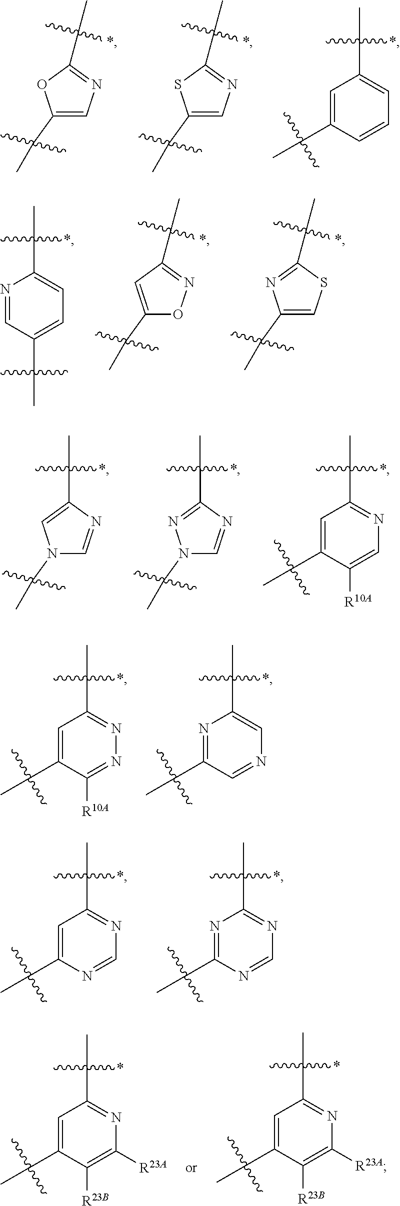

preferably R^{12A} is

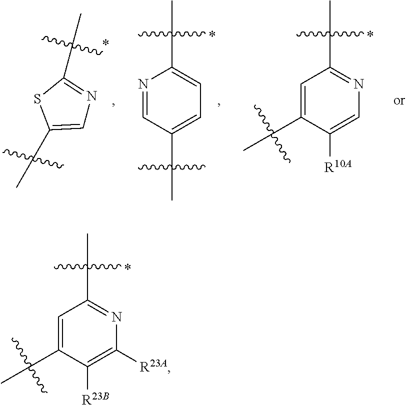

more preferably R^{12A} is

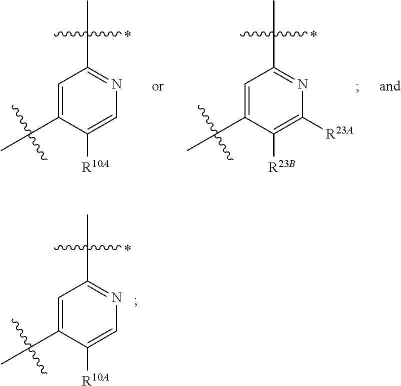

even more preferably R^{12A} is
wherein the * signifies the attachment of R^{12A} to the carbonyl carbon of Formula (III) and the other attachment point signifies the attachment of R^{12A} to the phenyl ring of R^{1A}; in

In a preferred embodiment of the third aspect R^{10A} is —H or —CH₃.

In a preferred embodiment R^{23A} and R^{23B} are independently —H, —CH₃ or —OCH₃.

In an preferred embodiment R^{13A} is as defined for R¹³ in in the third aspect and is —H, —CH₃, —F, —OCH₃, —CHCH₃OH, —SCH₃O₂, 2-methoxyethoxy, phenyl or oxolan-3-yloxy; more preferably H or —CH₃, even more preferably H.

In preferred embodiment of the third aspect R^{14A} is —H, —CH₃, —OH, —F, —OCH₃, cyclopropoxy, —OCH₂CH₂OCH₃, —OCH₂CH₂OH, —OCH(CH₃)₂, —OCH₂CH₂OCH(CH₃)₂, —O(CH₂CH₂O)₇CH₃, —O(CH₂CH₂O)₇H,

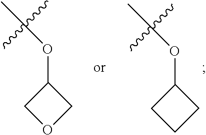 [SMILES: *OC1COC1 ; *OC1CCC1]

more preferably —H, —OH, —OCH(CH₃)₂, —OCH₂CH₂OCH(CH₃)₂ or —F; even more preferably —H.

In a preferred embodiment R^{15A} is as defined for R¹⁵ in the third aspect and is —H, —CHF₂, —CH₂F, —CF₃, —C₁-C₆alkyl, or C₂-C₆ fluoroalkyl; more preferably —H.

In a preferred embodiment R^{17A} is as defined for R¹⁷ in the third aspect and is

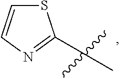 [SMILES: *c1nccs1]

cyclohexyl,

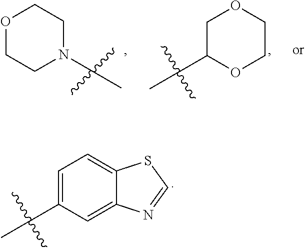 [SMILES: *N1CCOCC1 ; *C1COCCO1 ; *c1ccc2scnc2c1]

In a preferred embodiment R^{19A} is as defined for R¹⁹ in the third aspect and is —H, —C(CH₃)₃,

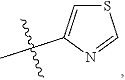 [SMILES: *c1cscn1]

cyclohexyl,

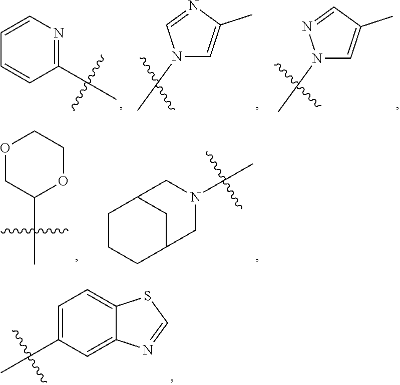

2,3,4,5,6-deuterated phenyl.

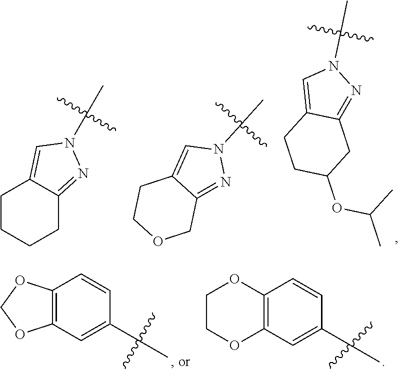

In preferred embodiments R^{20A} is —H or —CH₃.

In preferred embodiments R^{2A} is defined as R² in the third aspect; more preferably R^{2A} is

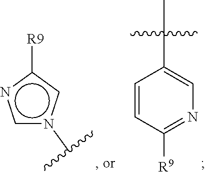

and R⁹ is CH₃ or CF₃;

In preferred embodiments R^{3A} is defined as R³ is the third aspect; more preferably R^{3A} is

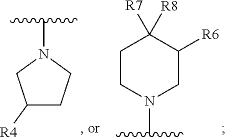

even more preferably

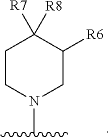

In preferred embodiments R⁴ is —NH₂ or —NHCH₃;

In preferred embodiments R⁶ is —NH₂, —OH, —NHCOR¹⁶ wherein R¹⁶ is a previously defined, or —NHSO₂CH₂CH₃; more preferably R⁶ is —NH₂ or —OH; even more preferably —NH₂.

In preferred embodiments R⁷ is —H, —OH, or —F; more preferably —H or —F, even more preferably —F.

In preferred embodiments R^{a} is —H or —F; more preferably —F.

In a preferred embodiment of the third aspect there is provided a compound or a pharmaceutically acceptable salt, solvate, prodrug or polymorph or deuterated analogue thereof, wherein the compound is a compound of formula (IIIa)

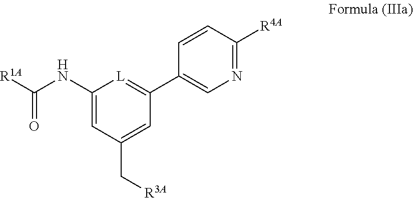

wherein
L is CH or N;
R^{1A} and R^{3A} are as previously defined; and
R^{4A} is CH₃ or CF₃;

In a preferred embodiment of the second aspect there is provided a compound or a pharmaceutically acceptable salt, solvate, prodrug or polymorph or deuterated analogue thereof, wherein the compound is a compound of formula (IIIb)

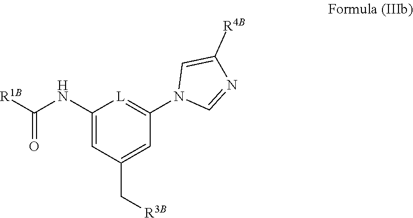

R^{4B} is CH₃ or CF₃; more preferably CH₃;
R^{1B} is defined as R^{1A} as previously defined; and
R^{3B} is defined as R^{3A} as previously defined.

In preferred embodiments L is CH or N; in some embodiments L is preferably CH; in some embodiments L is preferably N.

In a preferred embodiment of the third aspect there is provided a compound or a pharmaceutically acceptable salt, solvate, prodrug or polymorph or deuterated analogue thereof, wherein the compound is a compound of formula (IVa)

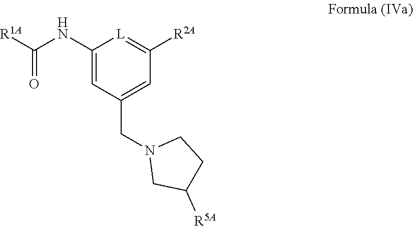

wherein
R^{1A} and R^{2A} are as previously defined.

In preferred embodiments L is CH or N; in some embodiments L is preferably CH; in some embodiments L is preferably N.

In preferred embodiments R^{5A} is —NH₂ or —NHCH₃; more preferably —NH₂.

In a preferred embodiment of the third aspect there is provided a compound or a pharmaceutically acceptable salt, solvate, prodrug or polymorph or deuterated analogue thereof, wherein the compound is a compound of formula (IVb)

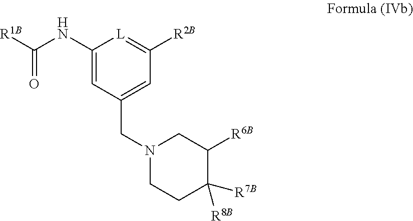

wherein R^{1B} and R^{2B} are as previously defined.

In preferred embodiments L is CH or N; in some embodiments L is preferably CH; in some embodiments L is preferably N.

In preferred embodiments R^{6B} is defined as for R⁶ in the third aspect; preferably —NH₂ or —NH₂CH₃, more preferably NH₂;

In preferred embodiments R^{7B} is defined as for R⁷ in the third aspect, preferably H or F; more preferably F;

In preferred embodiments R^{8B} is defined as for R⁷ in the third aspect; preferably H or F; more preferably F;

In a fourth aspect there is provided a compound or a pharmaceutically acceptable salt, solvate, prodrug or polymorph or deuterated analogue thereof wherein the compound is a compound of Formula (V)

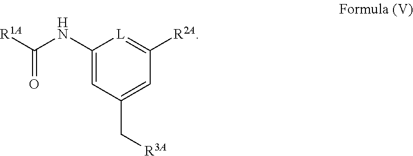

In preferred embodiments L is CH or N; in some embodiments L is more preferably CH; in some embodiments L is more preferably N.

In preferred embodiments R^{1A} is

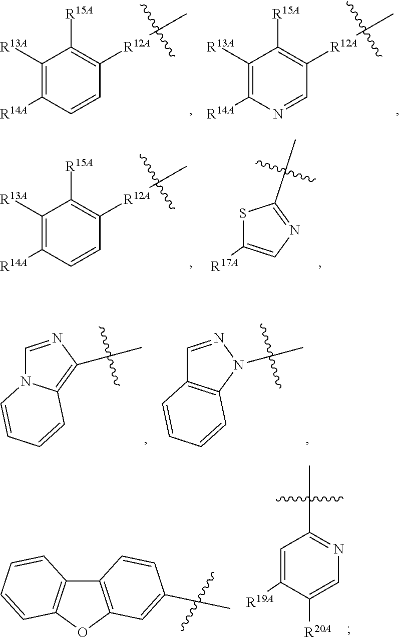

preferably R1A is

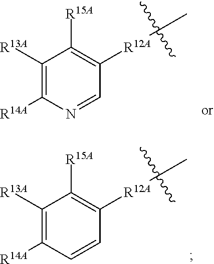

even more preferably R1A is

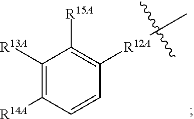

In preferred embodiments of the fourth aspect R^{12A} is selected from

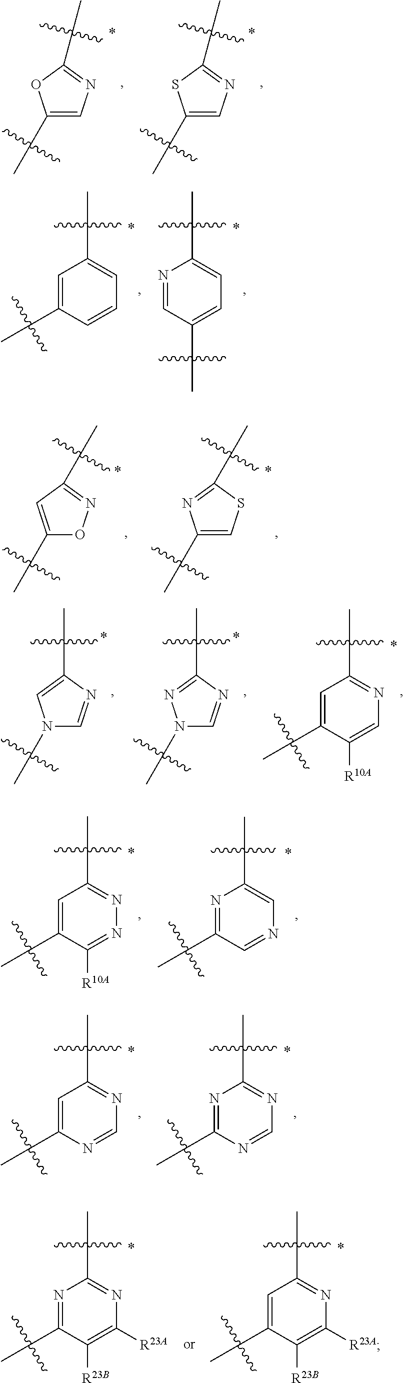

preferably R^{12A} is

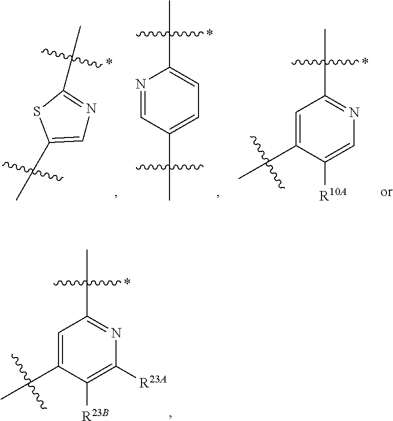

more preferably R^{12A} is

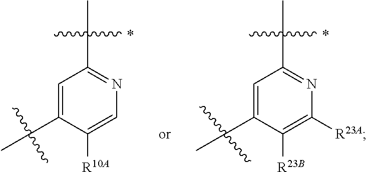

and even more preferably R^{12A} is

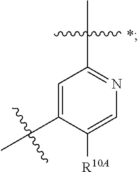

wherein the * signifies the attachment of R^{12A} to the carbonyl carbon of Formula (V) and the other attachment point signifies the attachment of R^{12A} to the phenyl ring of R^{1A};

In preferred embodiments R^{10A} is —H, —CH₃C₂-C₆alkyl, or C₁-C₆ fluoroalkyl; more preferably —H, —CH₃C₂-C₆alkyl, even more preferably H or CH₃.

In preferred embodiments R^{23A} and R^{32B} are independently —H, —CH₃, —OCH₃, C₂-C₆alkyl, or C₂-C₆alkoxy; more preferably —H, —CH₃ or —OCH₃.

In preferred embodiments R^{13A} is —H, —CH₃, —F, —OCH₃, —CH₂CH₃OH, —SCH₃O₂, 2-methoxyethoxy, phenyl, or oxolan-3-yloxy; more preferably H or —CH₃.

In preferred embodiments R^{14A} is —H, —CH₃, C₂-C₆ alkyl, —OH, —F, —Cl, —I, —Br, —OCH₃, —OC₂-C₆alkyl, cyclopropoxy, —OCH₂CH₂OCH₃, —OCH₂CH₂OH, —OCH(CH₃)₂, —OCH₂CH₂OCH(CH₃)₂, —O(CH₂CH₂O)₇CH₃, —O(CH₂CH₂O)₇H, O(CH2CH2O)ₙCH₃, or —O(CH2CH3O)ₙH, wherein n is 2, 3, 4, 5, or 6,

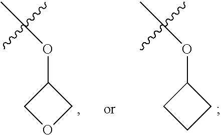 [SMILES: *OC1COC1 ; *OC1CCC1]

more preferably —H, —OH, —OCH(CH₃)₂, —OCH₂CH₂OCH(CH₃)₂ or —F; even more preferably —H.

In preferred embodiments R^{15A} is —H, —CHF₂, —CH₂F, —CF₃, —C₁-C₆alkyl, or C₂-C₆ fluoroalkyl; more preferably —H.

In preferred embodiments of the fourth aspect R^{17A} is

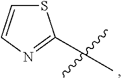 [SMILES: *c1nccs1]

cyclohexyl, C₃-C₅ cycloalkyl,

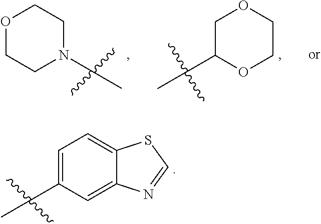 [SMILES: *N1CCOCC1 ; *C1COCCO1 ; *c1ccc2scnc2c1]

In preferred embodiments of the fourth aspect R^{19A} is —H, —C(CH₃)₃, C₁-C₃alkyl, C₅-C₆alkyl,

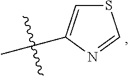 [SMILES: *c1cscn1]

cyclohexyl, C₃-C₅ cycloalkyl, C₁-C₆ fluoroalkyl,

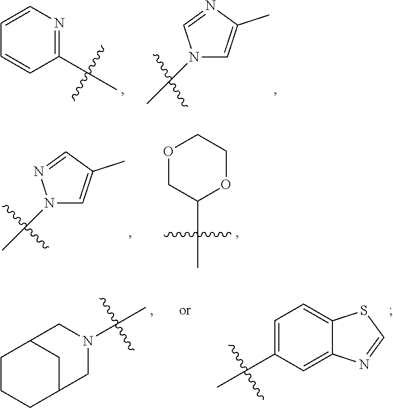

In preferred embodiments of the R^{20A} is —H, —F, —CH₃, C₂-C₆ alkyl C₁-C₆ fluoroalkyl, substituted C₁ alkyl, or substituted C₁-C₆ alkyl; more preferably —H or —CH₃.

In preferred embodiments of the fourth aspect
R^{2A} is selected from the group consisting of

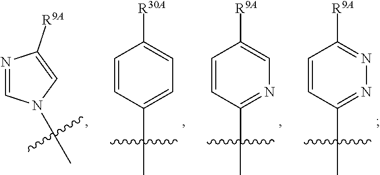

more preferably,

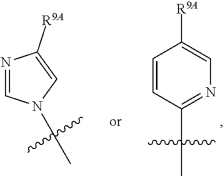

even more preferably

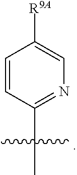

In preferred embodiments of the fourth aspect R^{9A} is —CH₃, —CF₃, C₂-C₆ alkyl, C₂-C₆ fluoroalkyl, —CH₂F, —CHF₂; more preferably —CH3, CH₂F, —CHF₂ or —CF₃, even more preferably —CH₃ or CF₃.

In preferred embodiments of the fourth aspect R^{30A} is H, —CH₃—C₂-C₆alkyl, or C₂-C₆ fluoroalkyl; more preferably —H or —CH₃ or —C₂-C₆alkyl; even more preferably —H or —CH₃.

In preferred embodiments of the fourth aspect R^{3A} is selected from the group consisting of —OCH₂CH₂NH₂,

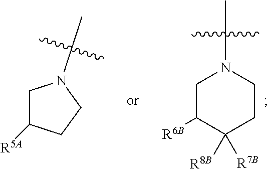

more preferably

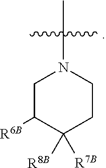

In preferred embodiments of the fourth aspect wherein R^{5A} is —NH₂, —NHCH₃ or —C₂-C₆alkylamino; more preferably —NH₂, —NHCH₃.

In preferred embodiments of the fourth aspect R^{6B} is —NH₂, —NH₂CH₃, C₂-C₆alkylamino, —OH, —NHCOR^{16B}, or —NHSO₂CH₂CH₃; more preferably-NH₂.

In preferred embodiments of the fourth aspect R^{16B} is —CH₂CH₃, —CH₃, —C₃-C₆alkyl,

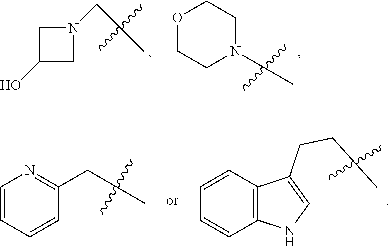 [SMILES: *CN1CC(O)C1 ; *N1CCOCC1 ; *Cc1ccccn1 ; *CCc1c[nH]c2ccccc12]

In preferred embodiments of the fourth aspect R^{7B} is —H, —OH, —F, —Cl, —Br, —I or —C₁-C₃alkyl; more preferably —H or —F, even more preferably —F.

In preferred embodiments of the fourth aspect R^{8B} is —H, —F, —Cl, —Br, —I or —C₁-C₃alkyl; more preferably —H or —F, even more preferably —F.

In a preferred embodiment of fourth aspect there is provided a compound or a pharmaceutically acceptable salt, solvate, prodrug or polymorph or deuterated analogue thereof wherein the compound is of formula (Va):

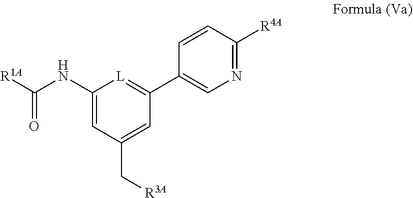

wherein R^{1A} and R^{3A} are as previously defined.

In preferred embodiments L is CH or N; in some embodiments L is more preferably CH; in some embodiments L is more preferably N.

In preferred embodiments R^{4A} is —CH₃, —CF₃, —C₂-C₆ alkyl or —C₂-C₆ fluoroalkyl; more preferably —CH₃ or —CF₃.

In preferred embodiments of the fourth aspect there is provided a compound or a pharmaceutically acceptable salt, solvate, prodrug or polymorph or deuterated analogue thereof wherein the compound is of formula (Vb):

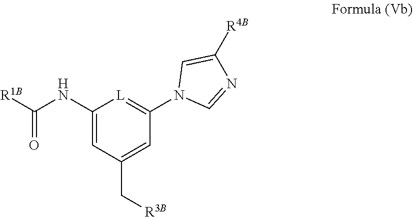

wherein R^{1B} and R^{3B} are as previously defined.

In preferred embodiments L is CH or N; in some embodiments L is more preferably CH; in some embodiments L is more preferably N.

In preferred embodiments R^{4B} is —CH₃, —CF₃, —C₂-C₆ alkyl or —C₂-C₆ fluoroalkyl; more preferably —CH₃.

In preferred embodiments of the fourth aspect there is provided a compound or a pharmaceutically acceptable salt, solvate, prodrug or polymorph or deuterated analogue thereof, wherein the compound is a compound of formula (VIa)

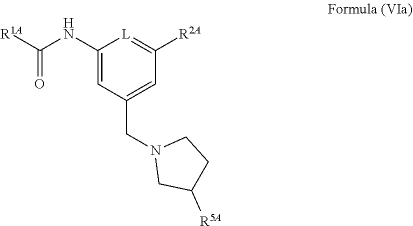

wherein R^{1A} and R^{2A} are as previously defined.

In preferred embodiments L is CH or N; in some embodiments L is more preferably CH; in some embodiments L is more preferably N.

In preferred embodiments R^{5A} is —NH₂, —NHCH₃ or —C₂-C₆ alkylamino; more preferably —NH₂ or —NHCH₃.

In preferred embodiments of the fourth aspect there is provided a compound or a pharmaceutically acceptable salt, solvate, prodrug or polymorph or deuterated analogue thereof, wherein the compound is a compound of formula (VIb)

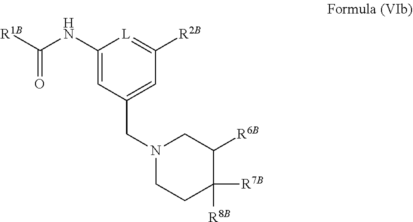

wherein R^{1B} and R^{2B} are as previously defined.

In preferred embodiments L is OH or N; more preferably OH.

In preferred embodiments R^{6B} is —NH₂, —NH₂CH₃, —C₂-C₆alkylamino; more preferably —NH₂.

In preferred embodiments R^{7B} is —H, —OH, —F, —Cl, —Br, or —I; more preferably —H or —F; even more preferably —F.

In preferred embodiments R^{8B} is —H, —F, —Cl, —Br, or —I; more preferably —H or —F; even more preferably —F.

### Compounds

In particular embodiments of the invention, the compound of Formula (I) has a structure selected from any one of the following in Table 1:

TABLE 1

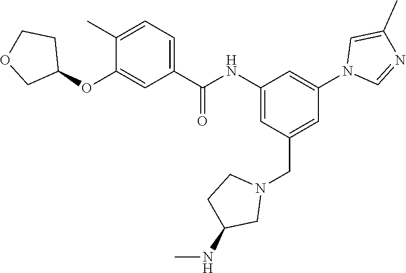 [SMILES: CN[C@H]1CCN(Cc2cc(NC(=O)c3ccc(C)c(O[C@@H]4CCOC4)c3)cc(-n3cnc(C)c3)c2)C1]

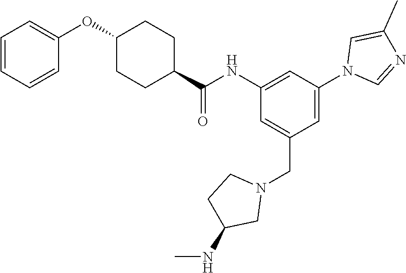 [SMILES: CN[C@H]1CCN(Cc2cc(NC(=O)[C@H]3CC[C@H](Oc4ccccc4)CC3)cc(-n3cnc(C)c3)c2)C1]

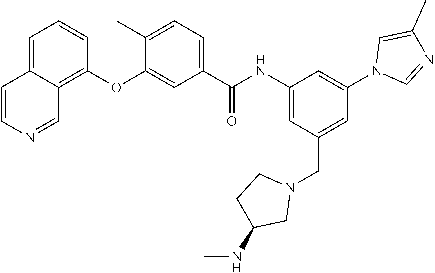 [SMILES: CN[C@H]1CCN(Cc2cc(NC(=O)c3ccc(C)c(Oc4cccc5ccncc45)c3)cc(-n3cnc(C)c3)c2)C1]

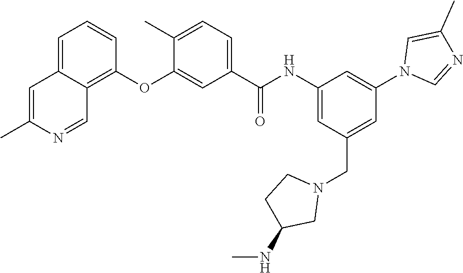 [SMILES: CN[C@H]1CCN(Cc2cc(NC(=O)c3ccc(C)c(Oc4cccc5cc(C)ncc45)c3)cc(-n3cnc(C)c3)c2)C1]

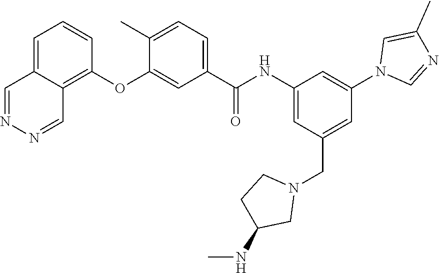 [SMILES: CN[C@H]1CCN(Cc2cc(NC(=O)c3ccc(C)c(Oc4cccc5cnncc45)c3)cc(-n3cnc(C)c3)c2)C1]

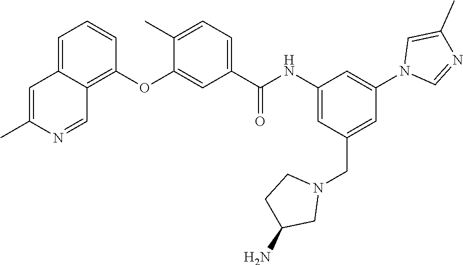 [SMILES: Cc1cc2cccc(Oc3cc(C(=O)Nc4cc(CN5CC[C@H](N)C5)cc(-n5cnc(C)c5)c4)ccc3C)c2cn1]

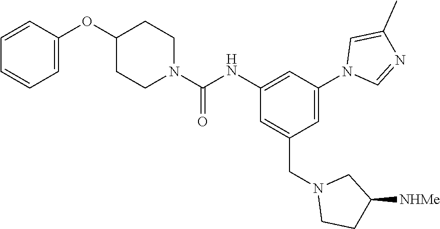 [SMILES: CN[C@H]1CCN(Cc2cc(NC(=O)N3CCC(Oc4ccccc4)CC3)cc(-n3cnc(C)c3)c2)C1]

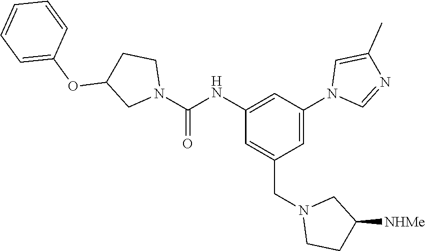 [SMILES: CN[C@H]1CCN(Cc2cc(NC(=O)N3CCC(Oc4ccccc4)C3)cc(-n3cnc(C)c3)c2)C1]

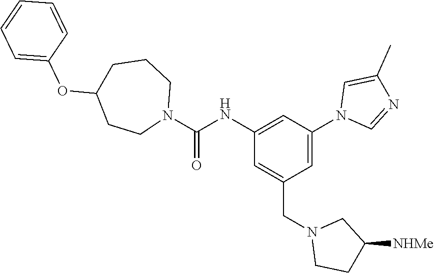 [SMILES: CN[C@H]1CCN(Cc2cc(NC(=O)N3CCCC(Oc4ccccc4)CC3)cc(-n3cnc(C)c3)c2)C1]

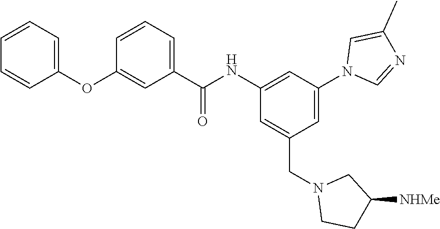 [SMILES: CN[C@H]1CCN(Cc2cc(NC(=O)c3cccc(Oc4ccccc4)c3)cc(-n3cnc(C)c3)c2)C1]

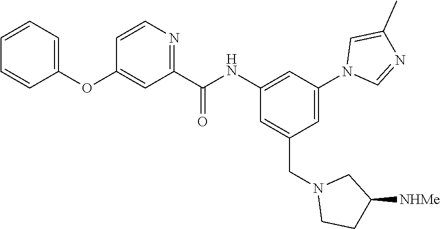 [SMILES: CN[C@H]1CCN(Cc2cc(NC(=O)c3cc(Oc4ccccc4)ccn3)cc(-n3cnc(C)c3)c2)C1]

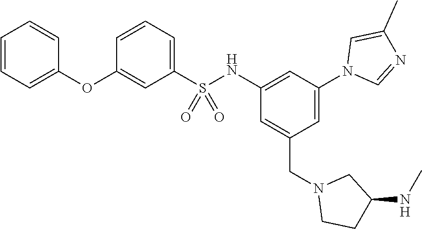 [SMILES: CN[C@H]1CCN(Cc2cc(NS(=O)(=O)c3cccc(Oc4ccccc4)c3)cc(-n3cnc(C)c3)c2)C1]

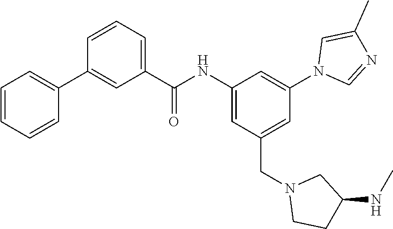 [SMILES: CN[C@H]1CCN(Cc2cc(NC(=O)c3cccc(-c4ccccc4)c3)cc(-n3cnc(C)c3)c2)C1]

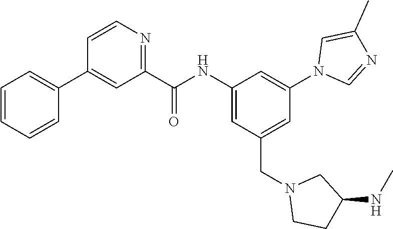 [SMILES: CN[C@H]1CCN(Cc2cc(NC(=O)c3cc(-c4ccccc4)ccn3)cc(-n3cnc(C)c3)c2)C1]

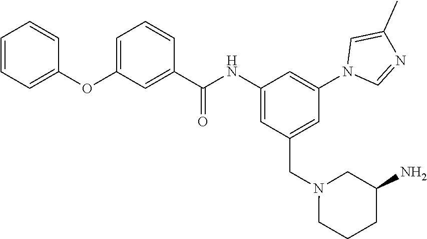 [SMILES: Cc1cn(-c2cc(CN3CCC[C@H](N)C3)cc(NC(=O)c3cccc(Oc4ccccc4)c3)c2)cn1]

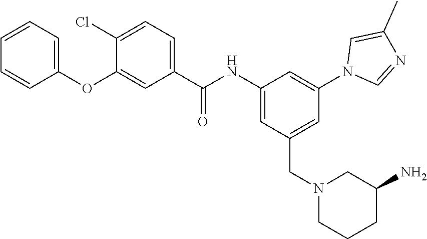 [SMILES: Cc1cn(-c2cc(CN3CCC[C@H](N)C3)cc(NC(=O)c3ccc(Cl)c(Oc4ccccc4)c3)c2)cn1]

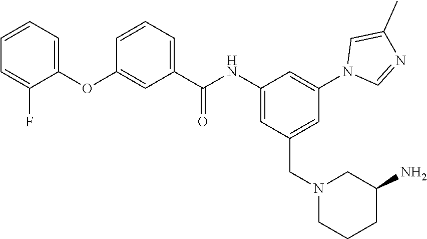 [SMILES: Cc1cn(-c2cc(CN3CCC[C@H](N)C3)cc(NC(=O)c3cccc(Oc4ccccc4F)c3)c2)cn1]

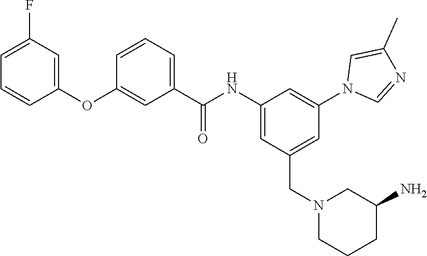 [SMILES: Cc1cn(-c2cc(CN3CCC[C@H](N)C3)cc(NC(=O)c3cccc(Oc4cccc(F)c4)c3)c2)cn1]

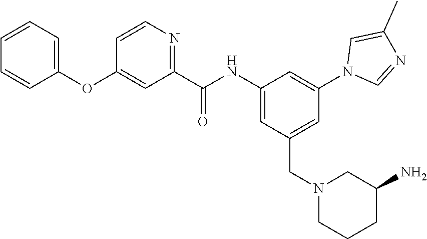 [SMILES: Cc1cn(-c2cc(CN3CCC[C@H](N)C3)cc(NC(=O)c3cc(Oc4ccccc4)ccn3)c2)cn1]

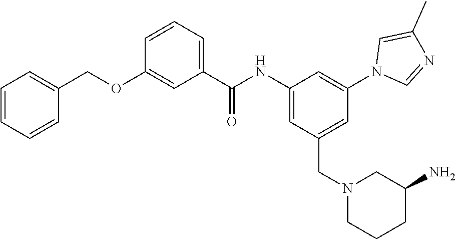 [SMILES: Cc1cn(-c2cc(CN3CCC[C@H](N)C3)cc(NC(=O)c3cccc(OCc4ccccc4)c3)c2)cn1]

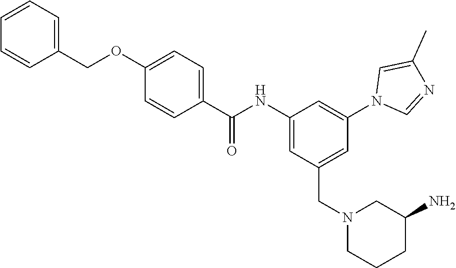 [SMILES: Cc1cn(-c2cc(CN3CCC[C@H](N)C3)cc(NC(=O)c3ccc(OCc4ccccc4)cc3)c2)cn1]

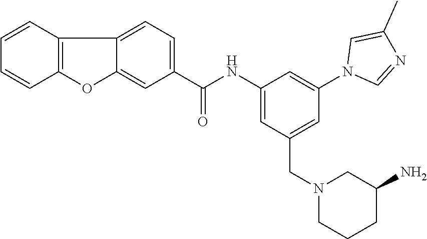 [SMILES: Cc1cn(-c2cc(CN3CCC[C@H](N)C3)cc(NC(=O)c3ccc4c(c3)oc3ccccc34)c2)cn1]

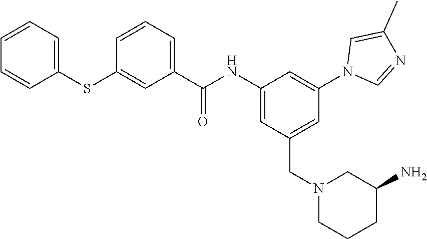 [SMILES: Cc1cn(-c2cc(CN3CCC[C@H](N)C3)cc(NC(=O)c3cccc(Sc4ccccc4)c3)c2)cn1]

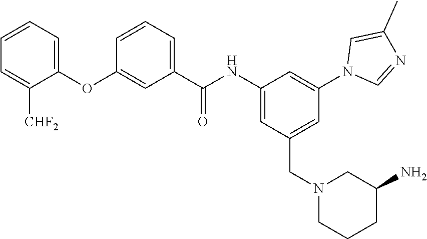 [SMILES: Cc1cn(-c2cc(CN3CCC[C@H](N)C3)cc(NC(=O)c3cccc(Oc4ccccc4C(F)F)c3)c2)cn1]

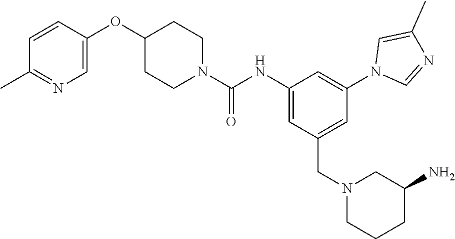 [SMILES: Cc1ccc(OC2CCN(C(=O)Nc3cc(CN4CCC[C@H](N)C4)cc(-n4cnc(C)c4)c3)CC2)cn1]

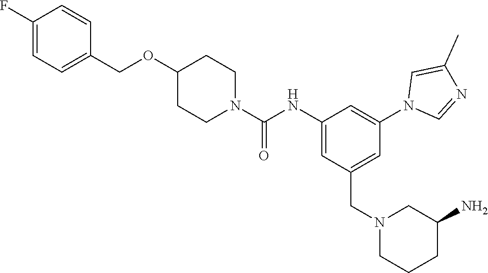 [SMILES: Cc1cn(-c2cc(CN3CCC[C@H](N)C3)cc(NC(=O)N3CCC(OCc4ccc(F)cc4)CC3)c2)cn1]

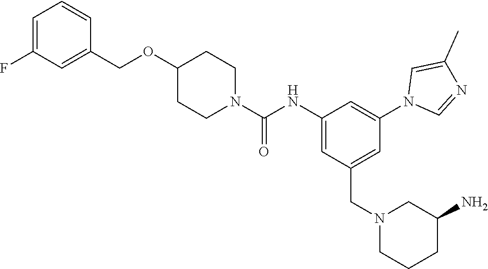 [SMILES: Cc1cn(-c2cc(CN3CCC[C@H](N)C3)cc(NC(=O)N3CCC(OCc4cccc(F)c4)CC3)c2)cn1]

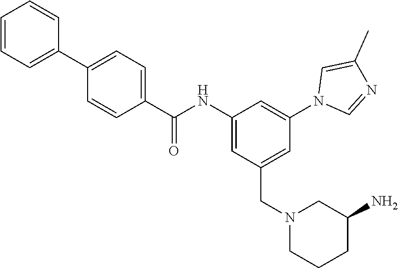 [SMILES: Cc1cn(-c2cc(CN3CCC[C@H](N)C3)cc(NC(=O)c3ccc(-c4ccccc4)cc3)c2)cn1]

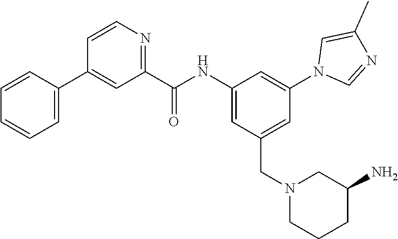 [SMILES: Cc1cn(-c2cc(CN3CCC[C@H](N)C3)cc(NC(=O)c3cc(-c4ccccc4)ccn3)c2)cn1]

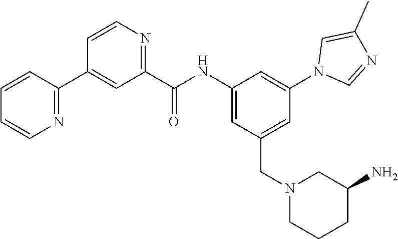 [SMILES: Cc1cn(-c2cc(CN3CCC[C@H](N)C3)cc(NC(=O)c3cc(-c4ccccn4)ccn3)c2)cn1]

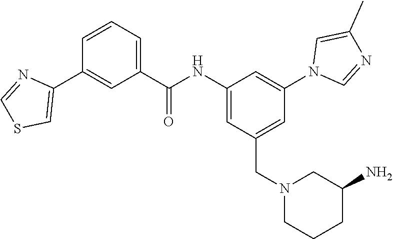 [SMILES: Cc1cn(-c2cc(CN3CCC[C@H](N)C3)cc(NC(=O)c3cccc(-c4cscn4)c3)c2)cn1]

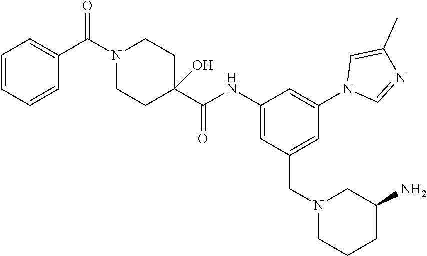 [SMILES: Cc1cn(-c2cc(CN3CCC[C@H](N)C3)cc(NC(=O)C3(O)CCN(C(=O)c4ccccc4)CC3)c2)cn1]

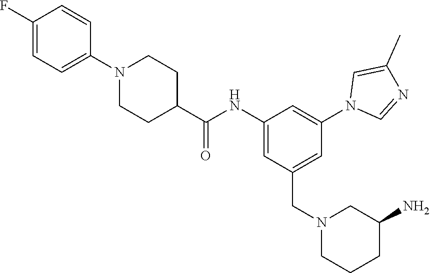 [SMILES: Cc1cn(-c2cc(CN3CCC[C@H](N)C3)cc(NC(=O)C3CCN(c4ccc(F)cc4)CC3)c2)cn1]

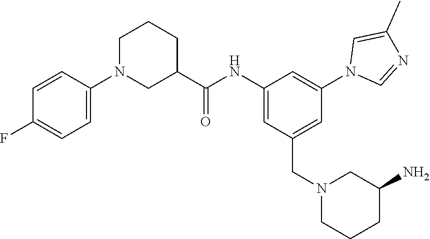 [SMILES: Cc1cn(-c2cc(CN3CCC[C@H](N)C3)cc(NC(=O)C3CCCN(c4ccc(F)cc4)C3)c2)cn1]

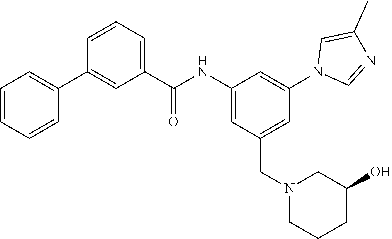 [SMILES: Cc1cn(-c2cc(CN3CCC[C@H](O)C3)cc(NC(=O)c3cccc(-c4ccccc4)c3)c2)cn1]

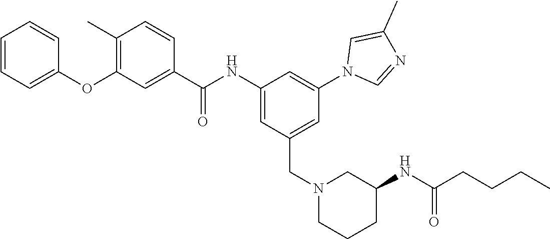 [SMILES: CCCCC(=O)N[C@H]1CCCN(Cc2cc(NC(=O)c3ccc(C)c(Oc4ccccc4)c3)cc(-n3cnc(C)c3)c2)C1]

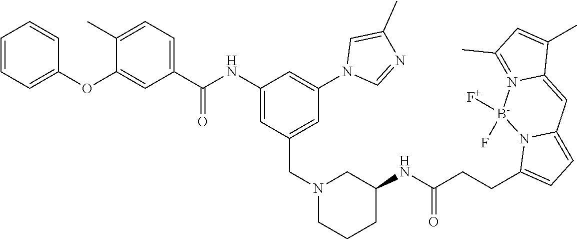

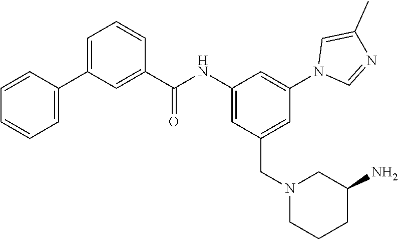 [SMILES: Cc1cn(-c2cc(CN3CCC[C@H](N)C3)cc(NC(=O)c3cccc(-c4ccccc4)c3)c2)cn1]

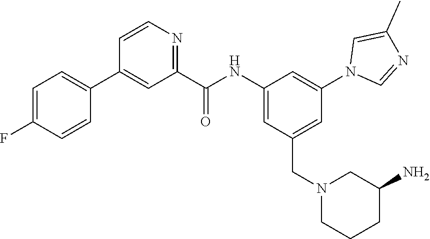 [SMILES: Cc1cn(-c2cc(CN3CCC[C@H](N)C3)cc(NC(=O)c3cc(-c4ccc(F)cc4)ccn3)c2)cn1]

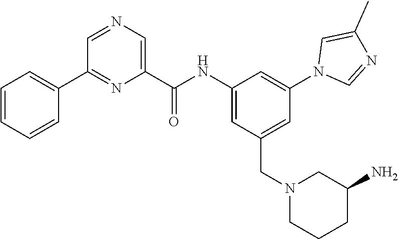 [SMILES: Cc1cn(-c2cc(CN3CCC[C@H](N)C3)cc(NC(=O)c3cncc(-c4ccccc4)n3)c2)cn1]

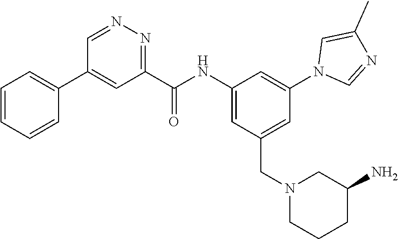 [SMILES: Cc1cn(-c2cc(CN3CCC[C@H](N)C3)cc(NC(=O)c3cc(-c4ccccc4)cnn3)c2)cn1]

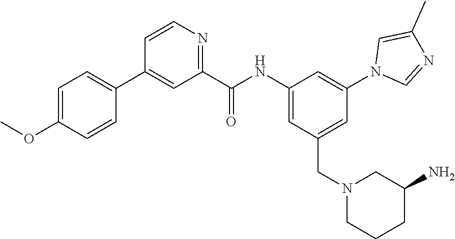 [SMILES: COc1ccc(-c2ccnc(C(=O)Nc3cc(CN4CCC[C@H](N)C4)cc(-n4cnc(C)c4)c3)c2)cc1]

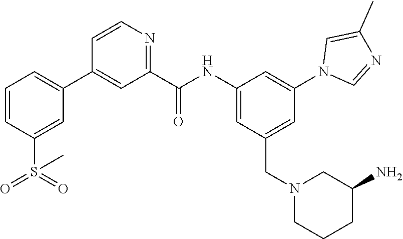 [SMILES: Cc1cn(-c2cc(CN3CCC[C@H](N)C3)cc(NC(=O)c3cc(-c4cccc(S(C)(=O)=O)c4)ccn3)c2)cn1]

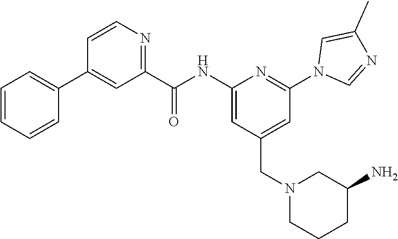 [SMILES: Cc1cn(-c2cc(CN3CCC[C@H](N)C3)cc(NC(=O)c3cc(-c4ccccc4)ccn3)n2)cn1]

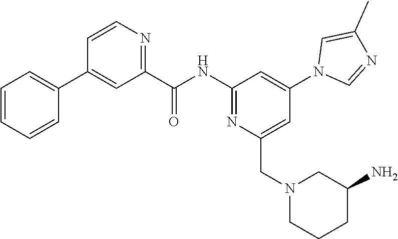 [SMILES: Cc1cn(-c2cc(CN3CCC[C@H](N)C3)nc(NC(=O)c3cc(-c4ccccc4)ccn3)c2)cn1]

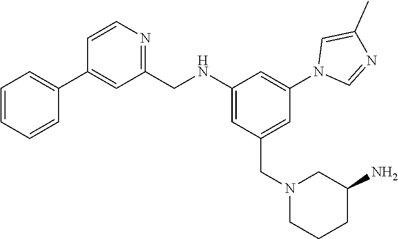 [SMILES: Cc1cn(-c2cc(CN3CCC[C@H](N)C3)cc(NCc3cc(-c4ccccc4)ccn3)c2)cn1]

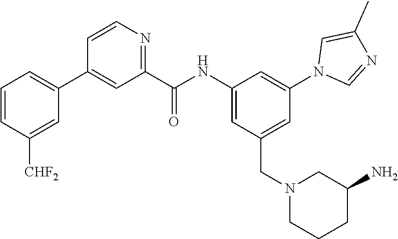 [SMILES: Cc1cn(-c2cc(CN3CCC[C@H](N)C3)cc(NC(=O)c3cc(-c4cccc(C(F)F)c4)ccn3)c2)cn1]

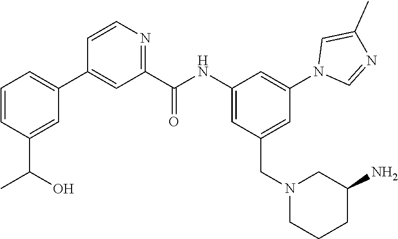 [SMILES: Cc1cn(-c2cc(CN3CCC[C@H](N)C3)cc(NC(=O)c3cc(-c4cccc(C(C)O)c4)ccn3)c2)cn1]

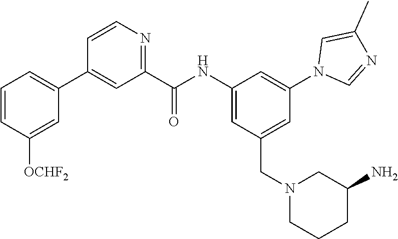 [SMILES: Cc1cn(-c2cc(CN3CCC[C@H](N)C3)cc(NC(=O)c3cc(-c4cccc(OC(F)F)c4)ccn3)c2)cn1]

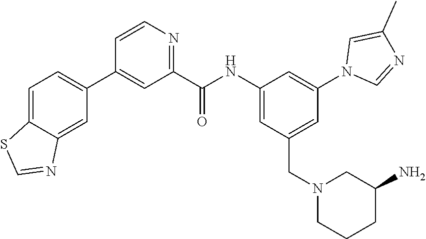 [SMILES: Cc1cn(-c2cc(CN3CCC[C@H](N)C3)cc(NC(=O)c3cc(-c4ccc5scnc5c4)ccn3)c2)cn1]

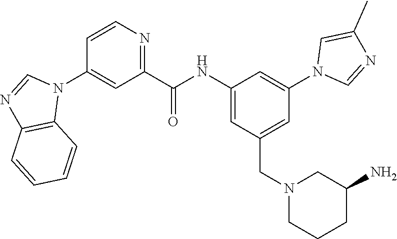 [SMILES: Cc1cn(-c2cc(CN3CCC[C@H](N)C3)cc(NC(=O)c3cc(-n4cnc5ccccc54)ccn3)c2)cn1]

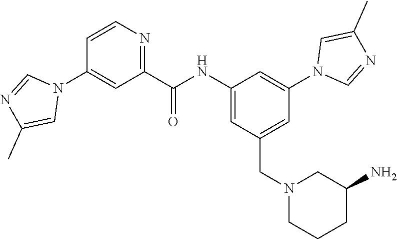 [SMILES: Cc1cn(-c2cc(CN3CCC[C@H](N)C3)cc(NC(=O)c3cc(-n4cnc(C)c4)ccn3)c2)cn1]

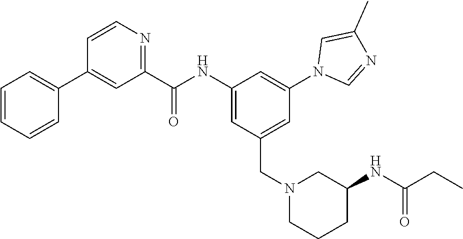 [SMILES: CCC(=O)N[C@H]1CCCN(Cc2cc(NC(=O)c3cc(-c4ccccc4)ccn3)cc(-n3cnc(C)c3)c2)C1]

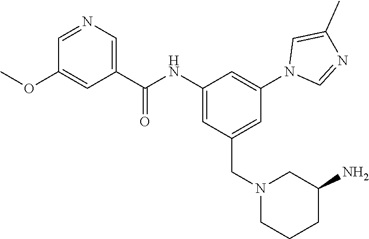 [SMILES: COc1cncc(C(=O)Nc2cc(CN3CCC[C@H](N)C3)cc(-n3cnc(C)c3)c2)c1]

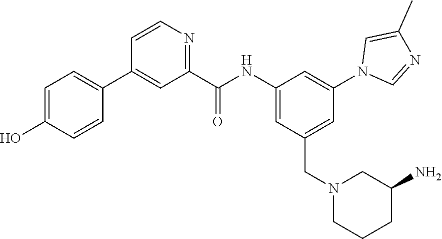 [SMILES: Cc1cn(-c2cc(CN3CCC[C@H](N)C3)cc(NC(=O)c3cc(-c4ccc(O)cc4)ccn3)c2)cn1]

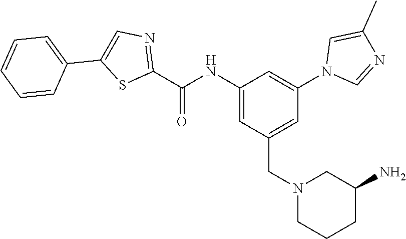 [SMILES: Cc1cn(-c2cc(CN3CCC[C@H](N)C3)cc(NC(=O)c3ncc(-c4ccccc4)s3)c2)cn1]

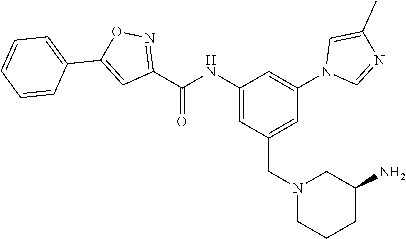 [SMILES: Cc1cn(-c2cc(CN3CCC[C@H](N)C3)cc(NC(=O)c3cc(-c4ccccc4)on3)c2)cn1]

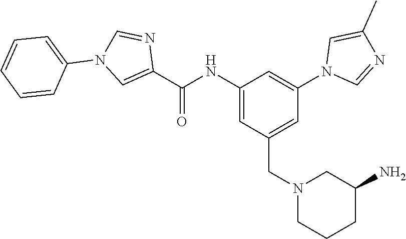 [SMILES: Cc1cn(-c2cc(CN3CCC[C@H](N)C3)cc(NC(=O)c3cn(-c4ccccc4)cn3)c2)cn1]

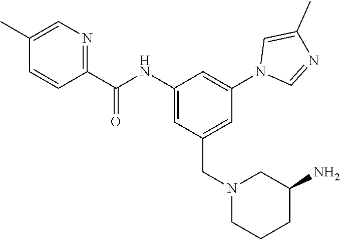 [SMILES: Cc1ccc(C(=O)Nc2cc(CN3CCC[C@H](N)C3)cc(-n3cnc(C)c3)c2)nc1]

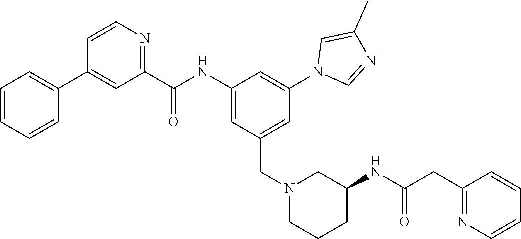 [SMILES: Cc1cn(-c2cc(CN3CCC[C@H](NC(=O)Cc4ccccn4)C3)cc(NC(=O)c3cc(-c4ccccc4)ccn3)c2)cn1]

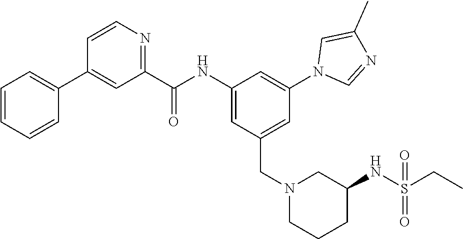 [SMILES: CCS(=O)(=O)N[C@H]1CCCN(Cc2cc(NC(=O)c3cc(-c4ccccc4)ccn3)cc(-n3cnc(C)c3)c2)C1]

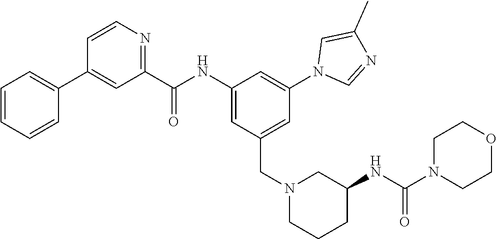 [SMILES: Cc1cn(-c2cc(CN3CCC[C@H](NC(=O)N4CCOCC4)C3)cc(NC(=O)c3cc(-c4ccccc4)ccn3)c2)cn1]

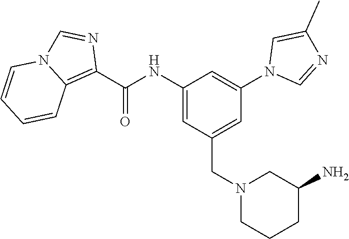 [SMILES: Cc1cn(-c2cc(CN3CCC[C@H](N)C3)cc(NC(=O)c3ncn4ccccc34)c2)cn1]

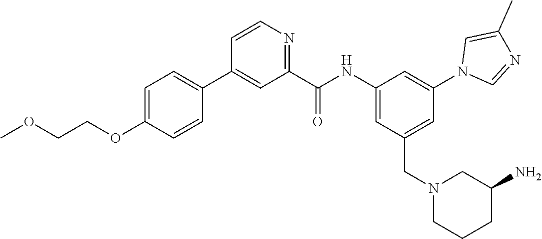 [SMILES: COCCOc1ccc(-c2ccnc(C(=O)Nc3cc(CN4CCC[C@H](N)C4)cc(-n4cnc(C)c4)c3)c2)cc1]

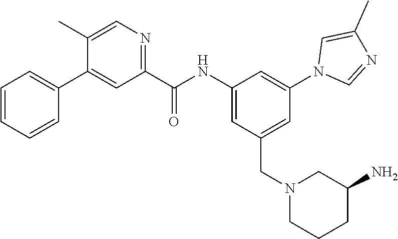 [SMILES: Cc1cn(-c2cc(CN3CCC[C@H](N)C3)cc(NC(=O)c3cc(-c4ccccc4)c(C)cn3)c2)cn1]

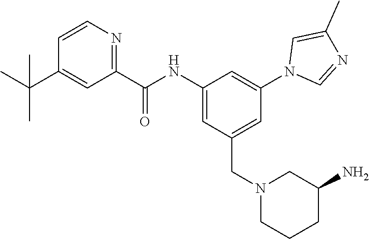 [SMILES: Cc1cn(-c2cc(CN3CCC[C@H](N)C3)cc(NC(=O)c3cc(C(C)(C)C)ccn3)c2)cn1]

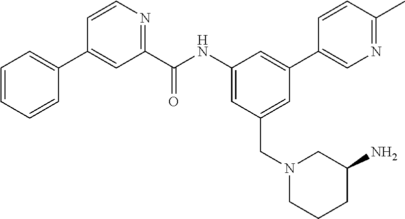 [SMILES: Cc1ccc(-c2cc(CN3CCC[C@H](N)C3)cc(NC(=O)c3cc(-c4ccccc4)ccn3)c2)cn1]

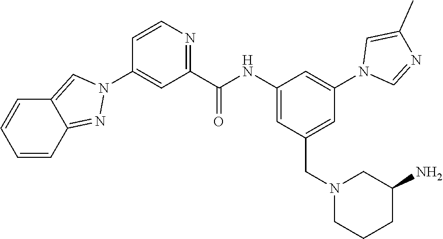 [SMILES: Cc1cn(-c2cc(CN3CCC[C@H](N)C3)cc(NC(=O)c3cc(-n4cc5ccccc5n4)ccn3)c2)cn1]

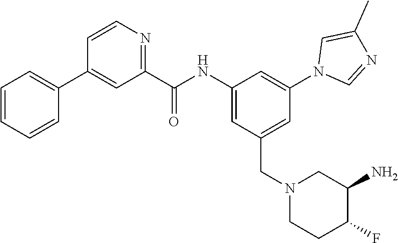 [SMILES: Cc1cn(-c2cc(CN3CC[C@@H](F)[C@H](N)C3)cc(NC(=O)c3cc(-c4ccccc4)ccn3)c2)cn1]

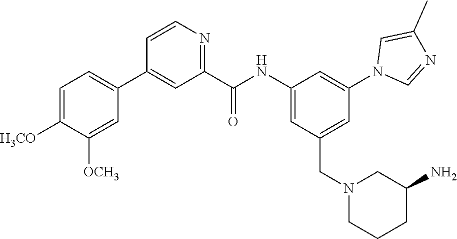 [SMILES: COc1ccc(-c2ccnc(C(=O)Nc3cc(CN4CCC[C@H](N)C4)cc(-n4cnc(C)c4)c3)c2)cc1OC]

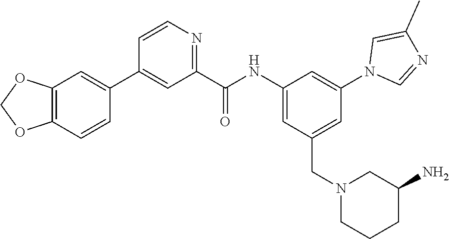 [SMILES: Cc1cn(-c2cc(CN3CCC[C@H](N)C3)cc(NC(=O)c3cc(-c4ccc5c(c4)OCO5)ccn3)c2)cn1]

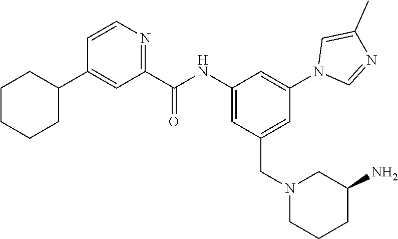 [SMILES: Cc1cn(-c2cc(CN3CCC[C@H](N)C3)cc(NC(=O)c3cc(C4CCCCC4)ccn3)c2)cn1]

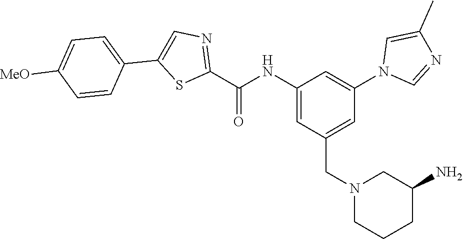 [SMILES: COc1ccc(-c2cnc(C(=O)Nc3cc(CN4CCC[C@H](N)C4)cc(-n4cnc(C)c4)c3)s2)cc1]

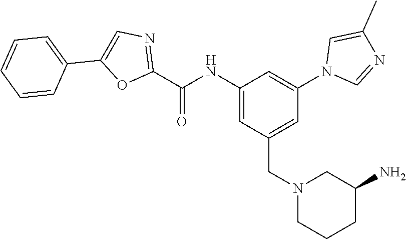 [SMILES: Cc1cn(-c2cc(CN3CCC[C@H](N)C3)cc(NC(=O)c3ncc(-c4ccccc4)o3)c2)cn1]

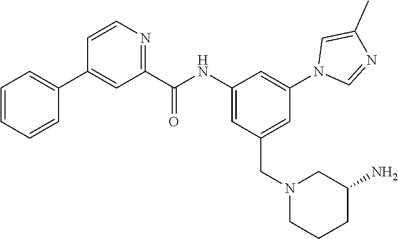 [SMILES: Cc1cn(-c2cc(CN3CCC[C@@H](N)C3)cc(NC(=O)c3cc(-c4ccccc4)ccn3)c2)cn1]

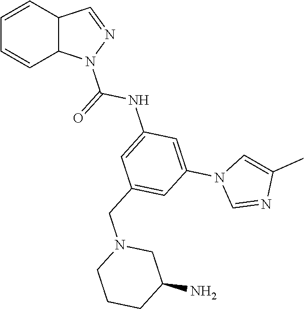 [SMILES: Cc1cn(-c2cc(CN3CCC[C@H](N)C3)cc(NC(=O)N3N=CC4C=CC=CC43)c2)cn1]

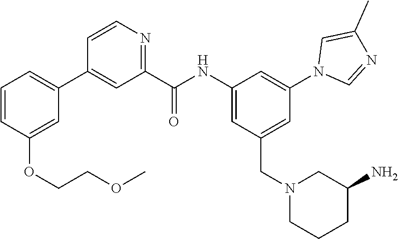 [SMILES: COCCOc1cccc(-c2ccnc(C(=O)Nc3cc(CN4CCC[C@H](N)C4)cc(-n4cnc(C)c4)c3)c2)c1]

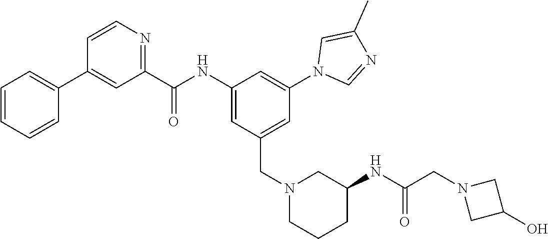 [SMILES: Cc1cn(-c2cc(CN3CCC[C@H](NC(=O)CN4CC(O)C4)C3)cc(NC(=O)c3cc(-c4ccccc4)ccn3)c2)cn1]

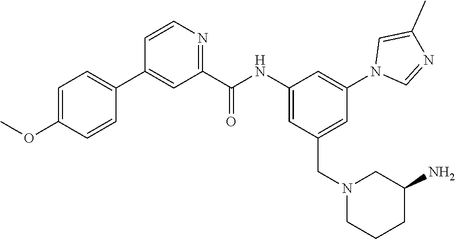 [SMILES: COc1ccc(-c2ccnc(C(=O)Nc3cc(CN4CCC[C@H](N)C4)cc(-n4cnc(C)c4)c3)c2)cc1]

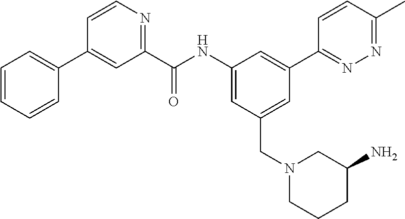 [SMILES: Cc1ccc(-c2cc(CN3CCC[C@H](N)C3)cc(NC(=O)c3cc(-c4ccccc4)ccn3)c2)nn1]

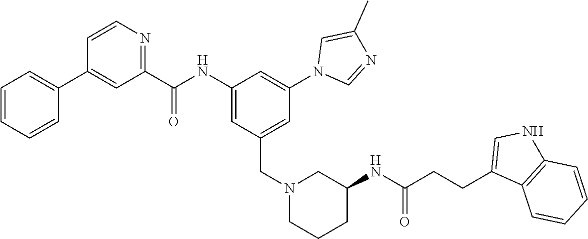 [SMILES: Cc1cn(-c2cc(CN3CCC[C@H](NC(=O)CCc4c[nH]c5ccccc45)C3)cc(NC(=O)c3cc(-c4ccccc4)ccn3)c2)cn1]

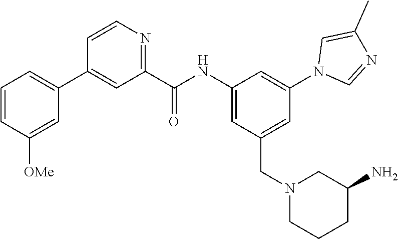 [SMILES: COc1cccc(-c2ccnc(C(=O)Nc3cc(CN4CCC[C@H](N)C4)cc(-n4cnc(C)c4)c3)c2)c1]

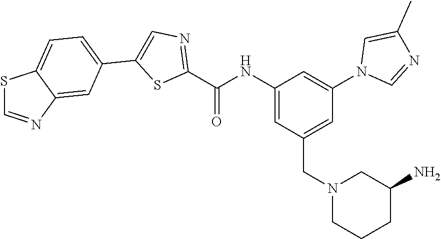 [SMILES: Cc1cn(-c2cc(CN3CCC[C@H](N)C3)cc(NC(=O)c3ncc(-c4ccc5scnc5c4)s3)c2)cn1]

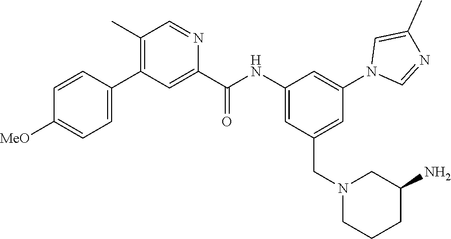 [SMILES: COc1ccc(-c2cc(C(=O)Nc3cc(CN4CCC[C@H](N)C4)cc(-n4cnc(C)c4)c3)ncc2C)cc1]

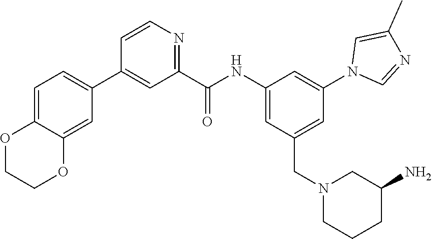 [SMILES: Cc1cn(-c2cc(CN3CCC[C@H](N)C3)cc(NC(=O)c3cc(-c4ccc5c(c4)OCCO5)ccn3)c2)cn1]

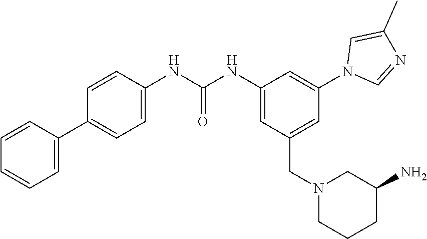 [SMILES: Cc1cn(-c2cc(CN3CCC[C@H](N)C3)cc(NC(=O)Nc3ccc(-c4ccccc4)cc3)c2)cn1]

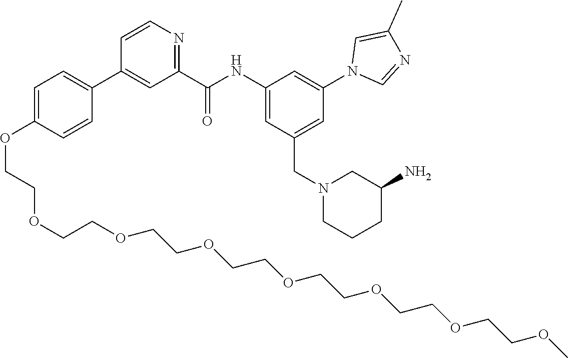 [SMILES: COCCOCCOCCOCCOCCOCCOCCOc1ccc(-c2ccnc(C(=O)Nc3cc(CN4CCC[C@H](N)C4)cc(-n4cnc(C)c4)c3)c2)cc1]

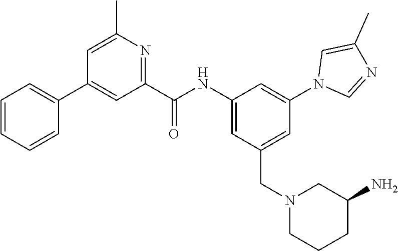 [SMILES: Cc1cn(-c2cc(CN3CCC[C@H](N)C3)cc(NC(=O)c3cc(-c4ccccc4)cc(C)n3)c2)cn1]

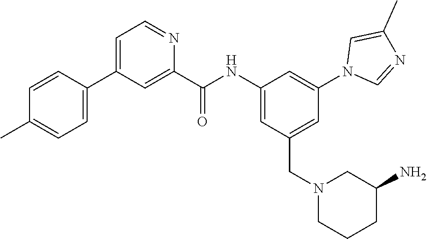 [SMILES: Cc1ccc(-c2ccnc(C(=O)Nc3cc(CN4CCC[C@H](N)C4)cc(-n4cnc(C)c4)c3)c2)cc1]

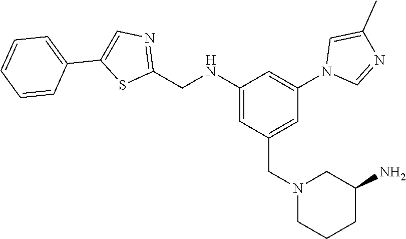 [SMILES: Cc1cn(-c2cc(CN3CCC[C@H](N)C3)cc(NCc3ncc(-c4ccccc4)s3)c2)cn1]

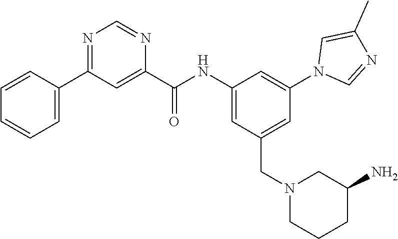 [SMILES: Cc1cn(-c2cc(CN3CCC[C@H](N)C3)cc(NC(=O)c3cc(-c4ccccc4)ncn3)c2)cn1]

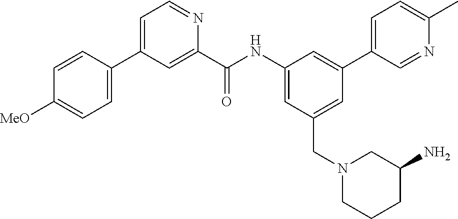 [SMILES: COc1ccc(-c2ccnc(C(=O)Nc3cc(CN4CCC[C@H](N)C4)cc(-c4ccc(C)nc4)c3)c2)cc1]

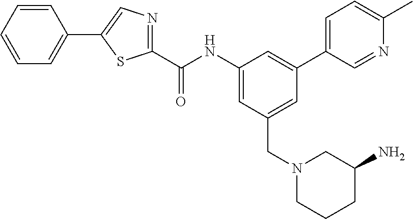 [SMILES: Cc1ccc(-c2cc(CN3CCC[C@H](N)C3)cc(NC(=O)c3ncc(-c4ccccc4)s3)c2)cn1]

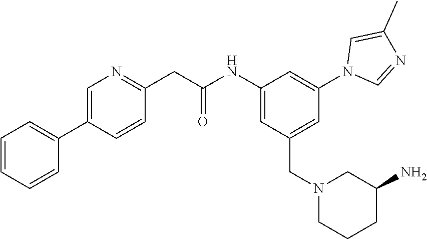 [SMILES: Cc1cn(-c2cc(CN3CCC[C@H](N)C3)cc(NC(=O)Cc3ccc(-c4ccccc4)cn3)c2)cn1]

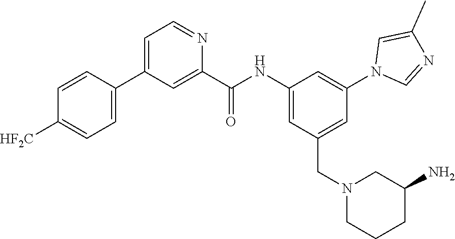

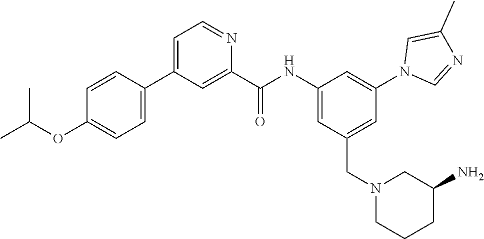 [SMILES: Cc1cn(-c2cc(CN3CCC[C@H](N)C3)cc(NC(=O)c3cc(-c4ccc(OC(C)C)cc4)ccn3)c2)cn1]

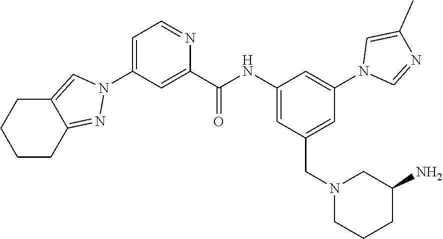 [SMILES: Cc1cn(-c2cc(CN3CCC[C@H](N)C3)cc(NC(=O)c3cc(-n4cc5c(n4)CCCC5)ccn3)c2)cn1]

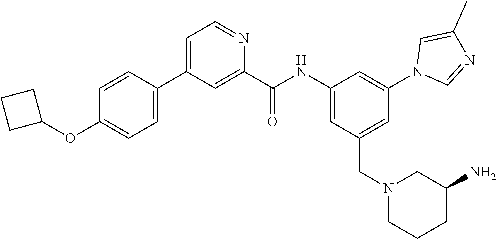 [SMILES: Cc1cn(-c2cc(CN3CCC[C@H](N)C3)cc(NC(=O)c3cc(-c4ccc(OC5CCC5)cc4)ccn3)c2)cn1]

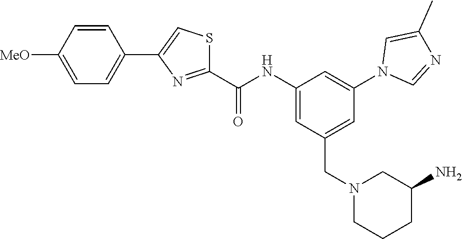 [SMILES: COc1ccc(-c2csc(C(=O)Nc3cc(CN4CCC[C@H](N)C4)cc(-n4cnc(C)c4)c3)n2)cc1]

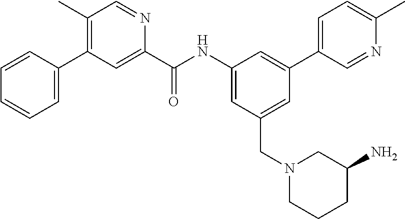 [SMILES: Cc1ccc(-c2cc(CN3CCC[C@H](N)C3)cc(NC(=O)c3cc(-c4ccccc4)c(C)cn3)c2)cn1]

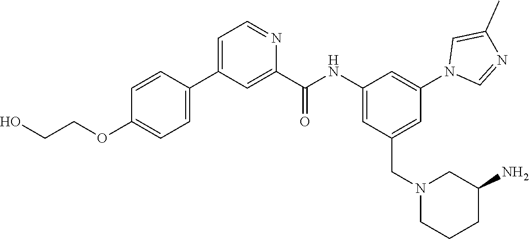 [SMILES: Cc1cn(-c2cc(CN3CCC[C@H](N)C3)cc(NC(=O)c3cc(-c4ccc(OCCO)cc4)ccn3)c2)cn1]

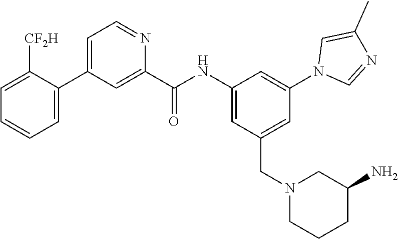 [SMILES: Cc1cn(-c2cc(CN3CCC[C@H](N)C3)cc(NC(=O)c3cc(-c4ccccc4C(F)F)ccn3)c2)cn1]

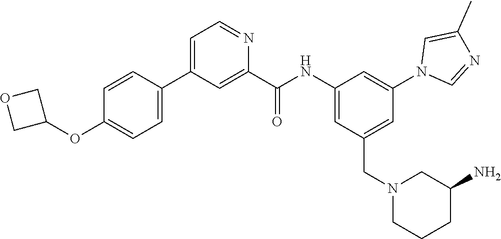 [SMILES: Cc1cn(-c2cc(CN3CCC[C@H](N)C3)cc(NC(=O)c3cc(-c4ccc(OC5COC5)cc4)ccn3)c2)cn1]

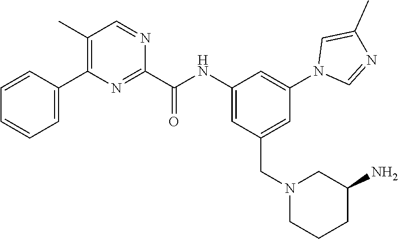 [SMILES: Cc1cn(-c2cc(CN3CCC[C@H](N)C3)cc(NC(=O)c3ncc(C)c(-c4ccccc4)n3)c2)cn1]

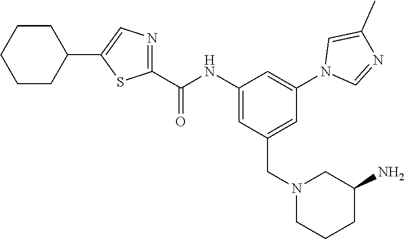 [SMILES: Cc1cn(-c2cc(CN3CCC[C@H](N)C3)cc(NC(=O)c3ncc(C4CCCCC4)s3)c2)cn1]

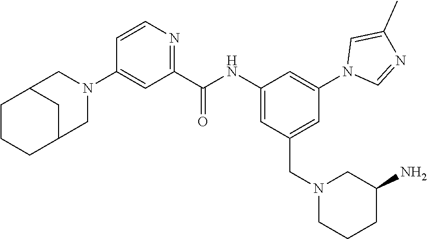 [SMILES: Cc1cn(-c2cc(CN3CCC[C@H](N)C3)cc(NC(=O)c3cc(N4CC5CCCC(C5)C4)ccn3)c2)cn1]

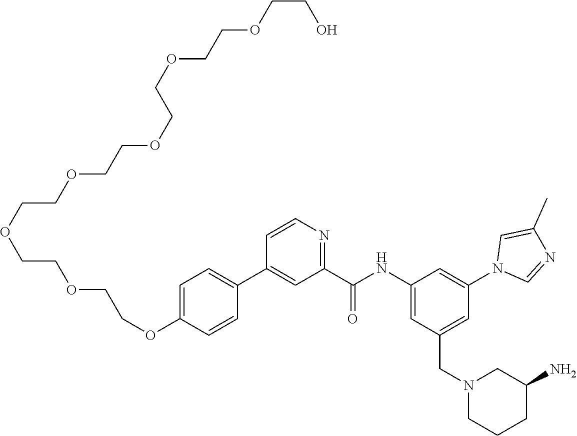 [SMILES: Cc1cn(-c2cc(CN3CCC[C@H](N)C3)cc(NC(=O)c3cc(-c4ccc(OCCOCCOCCOCCOCCOCCOCCO)cc4)ccn3)c2)cn1]

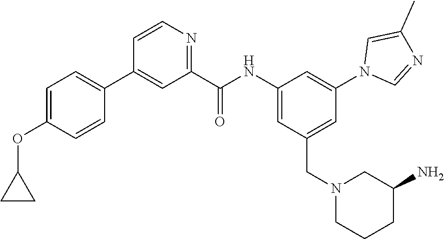 [SMILES: Cc1cn(-c2cc(CN3CCC[C@H](N)C3)cc(NC(=O)c3cc(-c4ccc(OC5CC5)cc4)ccn3)c2)cn1]

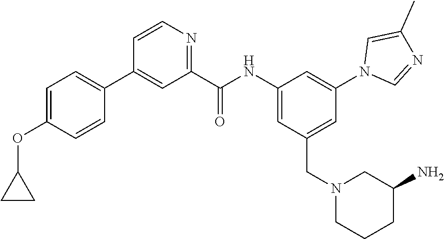 [SMILES: Cc1cn(-c2cc(CN3CCC[C@H](N)C3)cc(NC(=O)c3cc(-c4ccc(OC5CC5)cc4)ccn3)c2)cn1]

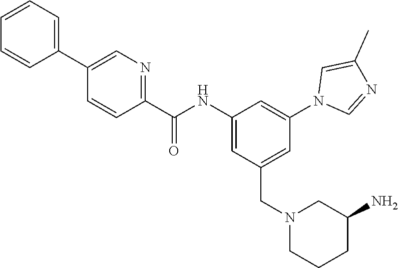 [SMILES: Cc1cn(-c2cc(CN3CCC[C@H](N)C3)cc(NC(=O)c3ccc(-c4ccccc4)cn3)c2)cn1]

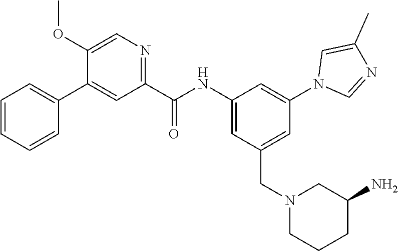 [SMILES: COc1cnc(C(=O)Nc2cc(CN3CCC[C@H](N)C3)cc(-n3cnc(C)c3)c2)cc1-c1ccccc1]

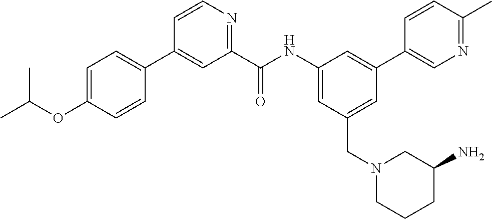 [SMILES: Cc1ccc(-c2cc(CN3CCC[C@H](N)C3)cc(NC(=O)c3cc(-c4ccc(OC(C)C)cc4)ccn3)c2)cn1]

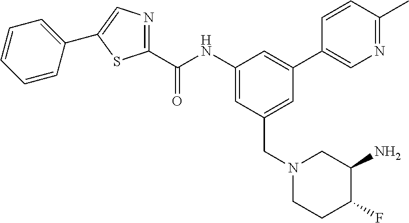 [SMILES: Cc1ccc(-c2cc(CN3CC[C@@H](F)[C@H](N)C3)cc(NC(=O)c3ncc(-c4ccccc4)s3)c2)cn1]

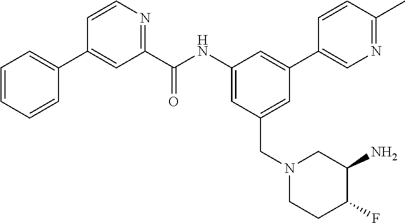 [SMILES: Cc1ccc(-c2cc(CN3CC[C@@H](F)[C@H](N)C3)cc(NC(=O)c3cc(-c4ccccc4)ccn3)c2)cn1]

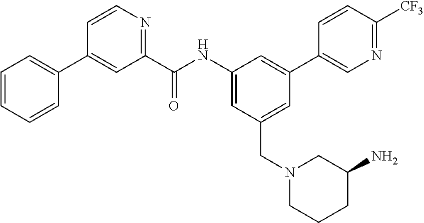 [SMILES: N[C@H]1CCCN(Cc2cc(NC(=O)c3cc(-c4ccccc4)ccn3)cc(-c3ccc(C(F)(F)F)nc3)c2)C1]

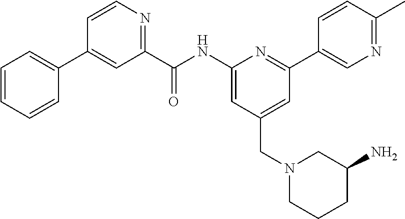 [SMILES: Cc1ccc(-c2cc(CN3CCC[C@H](N)C3)cc(NC(=O)c3cc(-c4ccccc4)ccn3)n2)cn1]

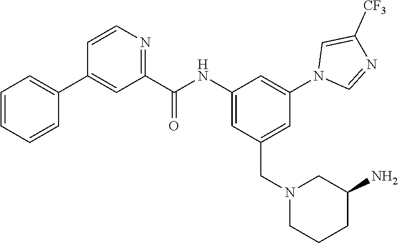 [SMILES: N[C@H]1CCCN(Cc2cc(NC(=O)c3cc(-c4ccccc4)ccn3)cc(-n3cnc(C(F)(F)F)c3)c2)C1]

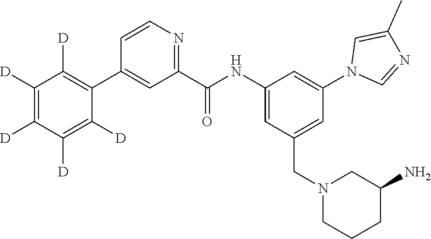 [SMILES: [2H]c1c([2H])c([2H])c(-c2ccnc(C(=O)Nc3cc(CN4CCC[C@H](N)C4)cc(-n4cnc(C)c4)c3)c2)c([2H])c1[2H]]

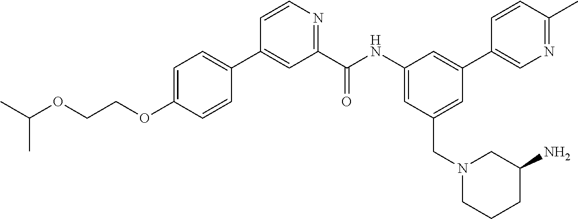 [SMILES: Cc1ccc(-c2cc(CN3CCC[C@H](N)C3)cc(NC(=O)c3cc(-c4ccc(OCCOC(C)C)cc4)ccn3)c2)cn1]

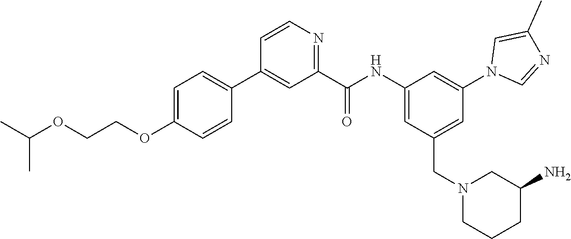 [SMILES: Cc1cn(-c2cc(CN3CCC[C@H](N)C3)cc(NC(=O)c3cc(-c4ccc(OCCOC(C)C)cc4)ccn3)c2)cn1]

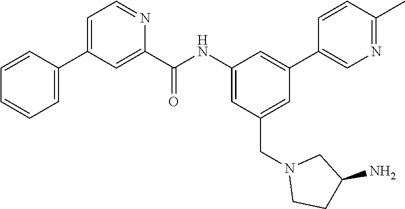 [SMILES: Cc1ccc(-c2cc(CN3CC[C@H](N)C3)cc(NC(=O)c3cc(-c4ccccc4)ccn3)c2)cn1]

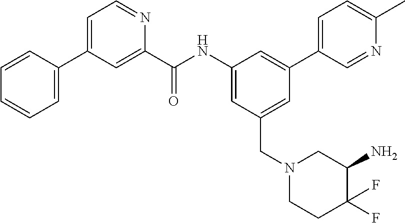 [SMILES: Cc1ccc(-c2cc(CN3CCC(F)(F)[C@H](N)C3)cc(NC(=O)c3cc(-c4ccccc4)ccn3)c2)cn1]

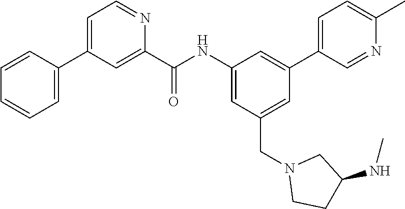 [SMILES: CN[C@H]1CCN(Cc2cc(NC(=O)c3cc(-c4ccccc4)ccn3)cc(-c3ccc(C)nc3)c2)C1]

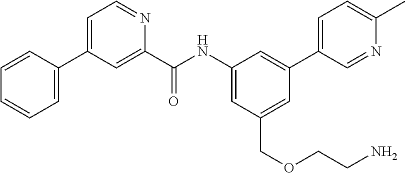 [SMILES: Cc1ccc(-c2cc(COCCN)cc(NC(=O)c3cc(-c4ccccc4)ccn3)c2)cn1]

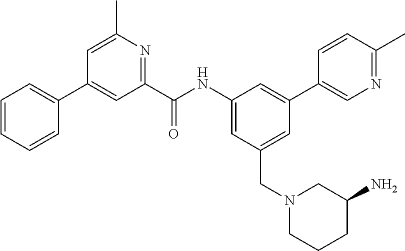 [SMILES: Cc1ccc(-c2cc(CN3CCC[C@H](N)C3)cc(NC(=O)c3cc(-c4ccccc4)cc(C)n3)c2)cn1]

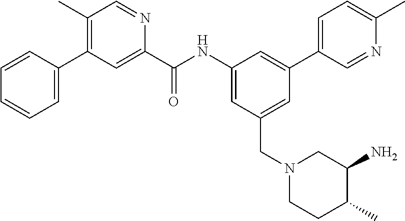 [SMILES: Cc1ccc(-c2cc(CN3CC[C@@H](C)[C@H](N)C3)cc(NC(=O)c3cc(-c4ccccc4)c(C)cn3)c2)cn1]

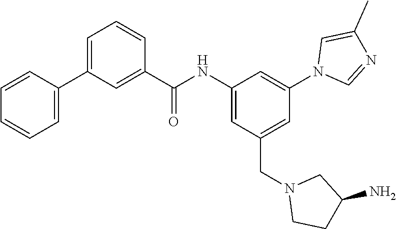 [SMILES: Cc1cn(-c2cc(CN3CC[C@H](N)C3)cc(NC(=O)c3cccc(-c4ccccc4)c3)c2)cn1]

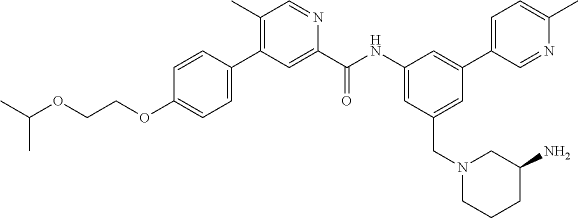 [SMILES: Cc1ccc(-c2cc(CN3CCC[C@H](N)C3)cc(NC(=O)c3cc(-c4ccc(OCCOC(C)C)cc4)c(C)cn3)c2)cn1]

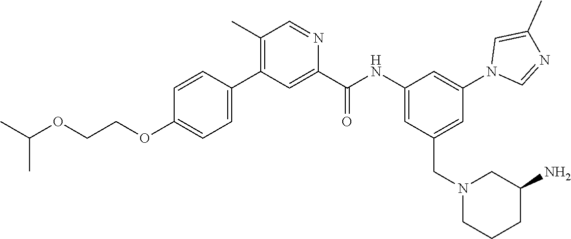 [SMILES: Cc1cn(-c2cc(CN3CCC[C@H](N)C3)cc(NC(=O)c3cc(-c4ccc(OCCOC(C)C)cc4)c(C)cn3)c2)cn1]

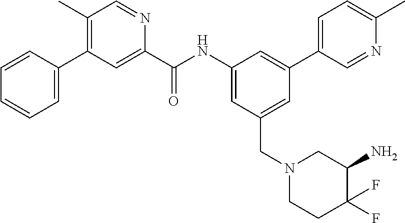 [SMILES: Cc1ccc(-c2cc(CN3CCC(F)(F)[C@H](N)C3)cc(NC(=O)c3cc(-c4ccccc4)c(C)cn3)c2)cn1]

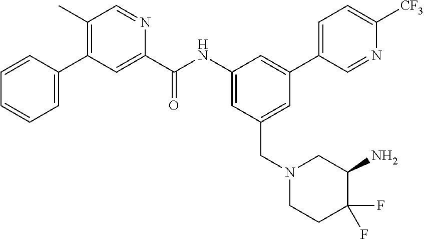 [SMILES: Cc1cnc(C(=O)Nc2cc(CN3CCC(F)(F)[C@H](N)C3)cc(-c3ccc(C(F)(F)F)nc3)c2)cc1-c1ccccc1]

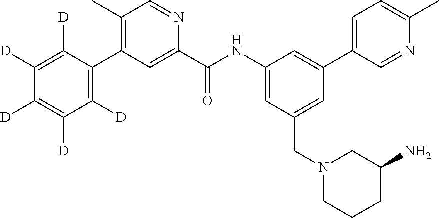 [SMILES: [2H]c1c([2H])c([2H])c(-c2cc(C(=O)Nc3cc(CN4CCC[C@H](N)C4)cc(-c4ccc(C)nc4)c3)ncc2C)c([2H])c1[2H]]

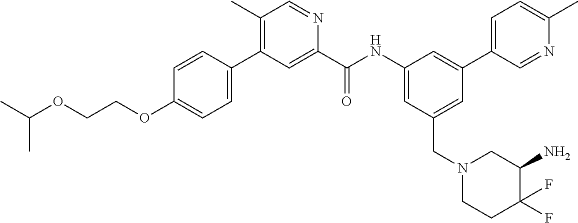 [SMILES: Cc1ccc(-c2cc(CN3CCC(F)(F)[C@H](N)C3)cc(NC(=O)c3cc(-c4ccc(OCCOC(C)C)cc4)c(C)cn3)c2)cn1]

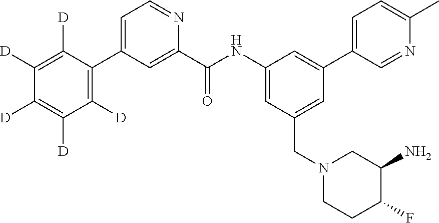 [SMILES: [2H]c1c([2H])c([2H])c(-c2ccnc(C(=O)Nc3cc(CN4CC[C@@H](F)[C@H](N)C4)cc(-c4ccc(C)nc4)c3)c2)c([2H])c1[2H]]

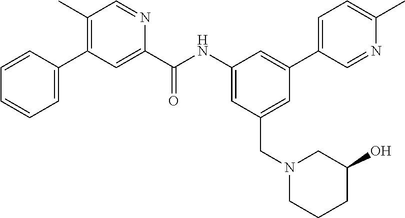 [SMILES: Cc1ccc(-c2cc(CN3CCC[C@H](O)C3)cc(NC(=O)c3cc(-c4ccccc4)c(C)cn3)c2)cn1]

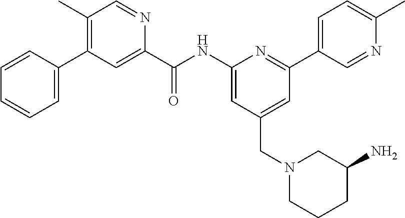 [SMILES: Cc1ccc(-c2cc(CN3CCC[C@H](N)C3)cc(NC(=O)c3cc(-c4ccccc4)c(C)cn3)n2)cn1]

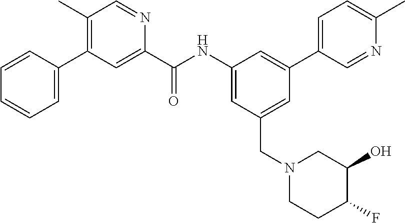 [SMILES: Cc1ccc(-c2cc(CN3CC[C@@H](F)[C@H](O)C3)cc(NC(=O)c3cc(-c4ccccc4)c(C)cn3)c2)cn1]

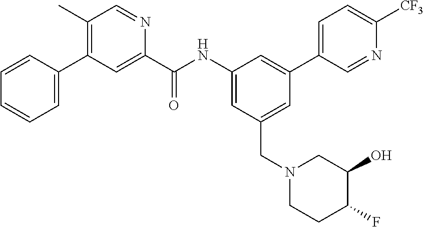 [SMILES: Cc1cnc(C(=O)Nc2cc(CN3CC[C@@H](F)[C@H](O)C3)cc(-c3ccc(C(F)(F)F)nc3)c2)cc1-c1ccccc1]

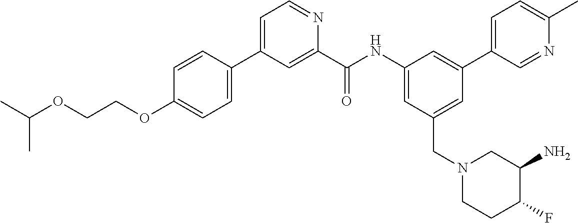 [SMILES: Cc1ccc(-c2cc(CN3CC[C@@H](F)[C@H](N)C3)cc(NC(=O)c3cc(-c4ccc(OCCOC(C)C)cc4)ccn3)c2)cn1]

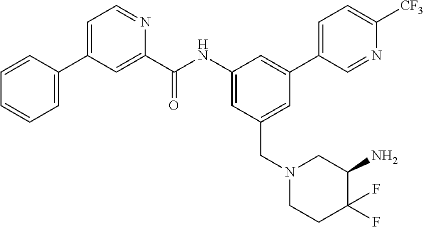 [SMILES: N[C@@H]1CN(Cc2cc(NC(=O)c3cc(-c4ccccc4)ccn3)cc(-c3ccc(C(F)(F)F)nc3)c2)CCC1(F)F]

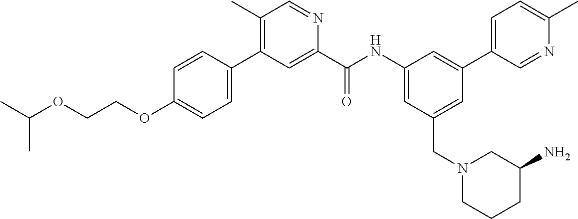 [SMILES: Cc1ccc(-c2cc(CN3CCC[C@H](N)C3)cc(NC(=O)c3cc(-c4ccc(OCCOC(C)C)cc4)c(C)cn3)c2)cn1]

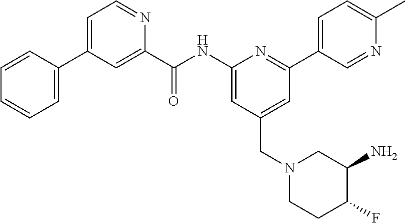 [SMILES: Cc1ccc(-c2cc(CN3CC[C@@H](F)[C@H](N)C3)cc(NC(=O)c3cc(-c4ccccc4)ccn3)n2)cn1]

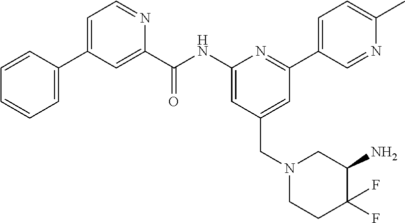 [SMILES: Cc1ccc(-c2cc(CN3CCC(F)(F)[C@H](N)C3)cc(NC(=O)c3cc(-c4ccccc4)ccn3)n2)cn1]

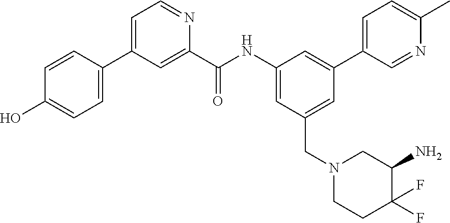 [SMILES: Cc1ccc(-c2cc(CN3CCC(F)(F)[C@H](N)C3)cc(NC(=O)c3cc(-c4ccc(O)cc4)ccn3)c2)cn1]

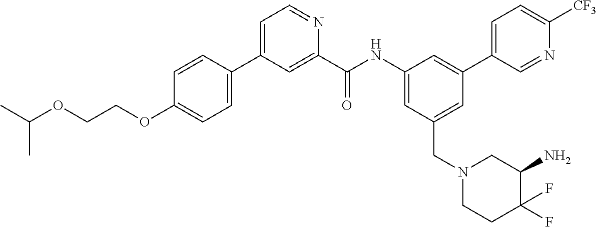 [SMILES: CC(C)OCCOc1ccc(-c2ccnc(C(=O)Nc3cc(CN4CCC(F)(F)[C@H](N)C4)cc(-c4ccc(C(F)(F)F)nc4)c3)c2)cc1]

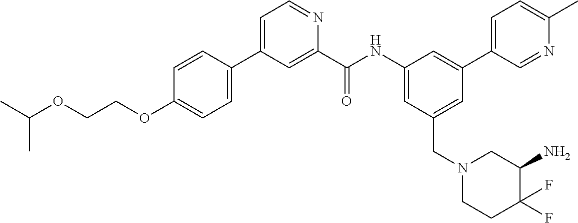 [SMILES: Cc1ccc(-c2cc(CN3CCC(F)(F)[C@H](N)C3)cc(NC(=O)c3cc(-c4ccc(OCCOC(C)C)cc4)ccn3)c2)cn1]

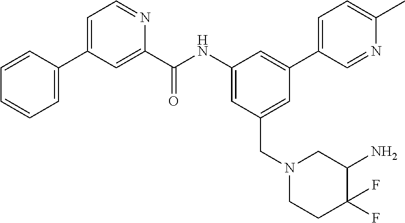 [SMILES: Cc1ccc(-c2cc(CN3CCC(F)(F)C(N)C3)cc(NC(=O)c3cc(-c4ccccc4)ccn3)c2)cn1]

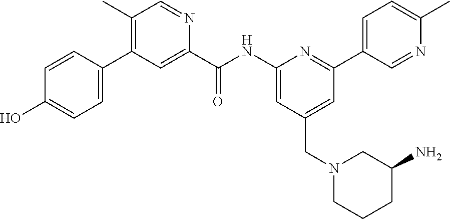 [SMILES: Cc1ccc(-c2cc(CN3CCC[C@H](N)C3)cc(NC(=O)c3cc(-c4ccc(O)cc4)c(C)cn3)n2)cn1]

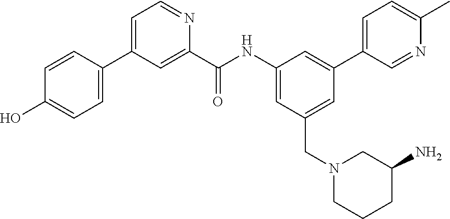 [SMILES: Cc1ccc(-c2cc(CN3CCC[C@H](N)C3)cc(NC(=O)c3cc(-c4ccc(O)cc4)ccn3)c2)cn1]

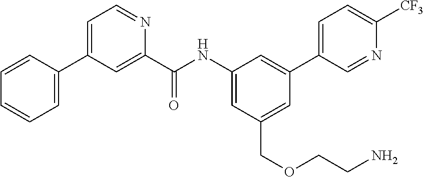 [SMILES: NCCOCc1cc(NC(=O)c2cc(-c3ccccc3)ccn2)cc(-c2ccc(C(F)(F)F)nc2)c1]

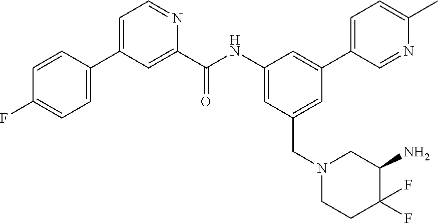 [SMILES: Cc1ccc(-c2cc(CN3CCC(F)(F)[C@H](N)C3)cc(NC(=O)c3cc(-c4ccc(F)cc4)ccn3)c2)cn1]

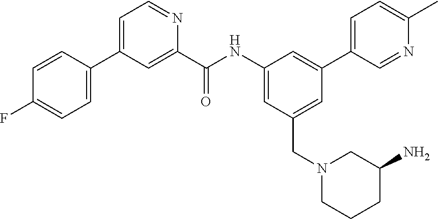 [SMILES: Cc1ccc(-c2cc(CN3CCC[C@H](N)C3)cc(NC(=O)c3cc(-c4ccc(F)cc4)ccn3)c2)cn1]

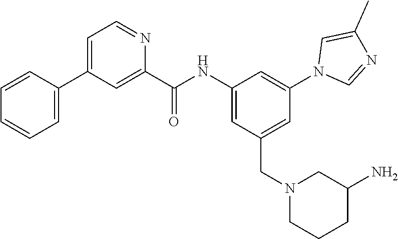 [SMILES: Cc1cn(-c2cc(CN3CCCC(N)C3)cc(NC(=O)c3cc(-c4ccccc4)ccn3)c2)cn1]

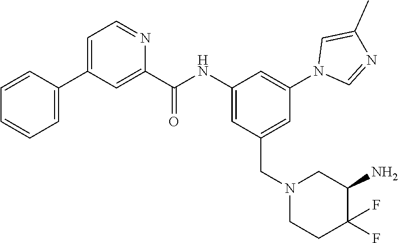 [SMILES: Cc1cn(-c2cc(CN3CCC(F)(F)[C@H](N)C3)cc(NC(=O)c3cc(-c4ccccc4)ccn3)c2)cn1]

In particular embodiments of the invention, the compound of Formula (I) has a structure selected from any one of the compounds in Table 1 other than compounds of the following structure:

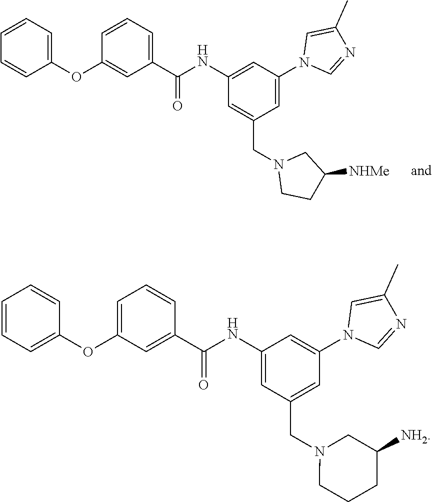 [SMILES: CN[C@H]1CCN(Cc2cc(NC(=O)c3cccc(Oc4ccccc4)c3)cc(-n3cnc(C)c3)c2)C1 ; "and" ; Cc1cn(-c2cc(CN3CCC[C@H](N)C3)cc(NC(=O)c3cccc(Oc4ccccc4)c3)c2)cn1]

In particular embodiments of the invention, the compound of Formula (I) has a structure selected from any one of the compounds in Table 1 other than compounds of the following structure:

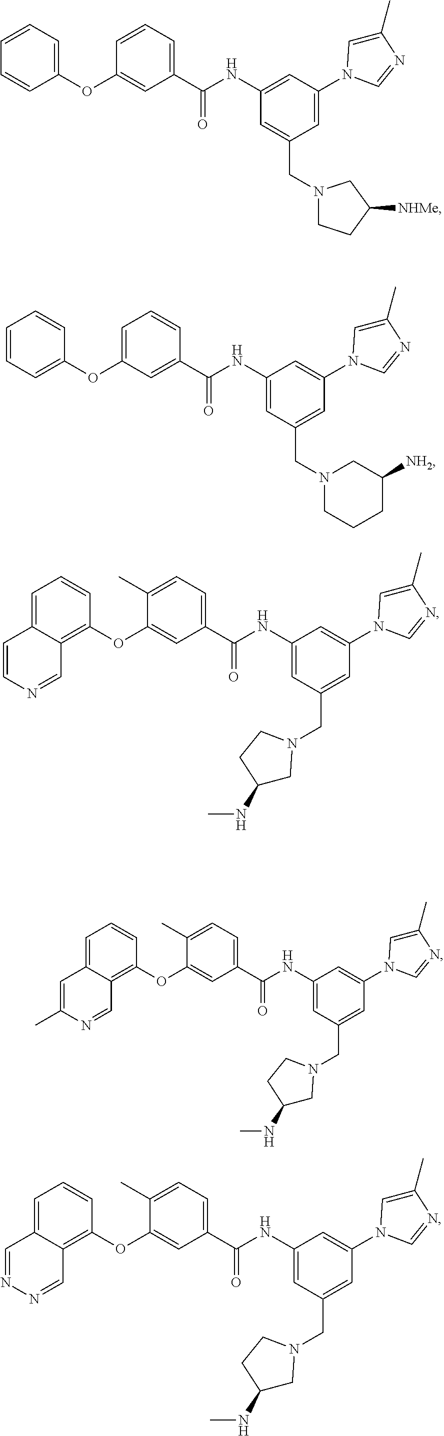 [SMILES: CN[C@H]1CCN(Cc2cc(NC(=O)c3cccc(Oc4ccccc4)c3)cc(-n3cnc(C)c3)c2)C1 ; Cc1cn(-c2cc(CN3CCC[C@H](N)C3)cc(NC(=O)c3cccc(Oc4ccccc4)c3)c2)cn1 ; CN[C@H]1CCN(Cc2cc(NC(=O)c3ccc(C)c(Oc4cccc5ccncc45)c3)cc(-n3cnc(C)c3)c2)C1 ; CN[C@H]1CCN(Cc2cc(NC(=O)c3ccc(C)c(Oc4cccc5cc(C)ncc45)c3)cc(-n3cnc(C)c3)c2)C1 ; CN[C@H]1CCN(Cc2cc(NC(=O)c3ccc(C)c(Oc4cccc5cnncc45)c3)cc(-n3cnc(C)c3)c2)C1]

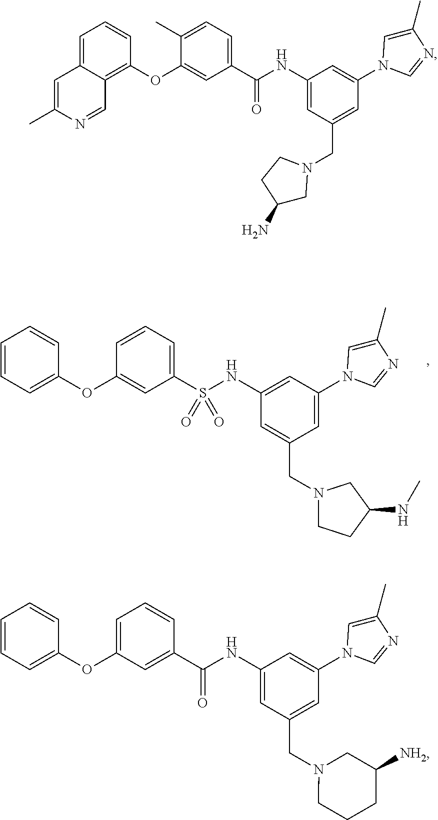

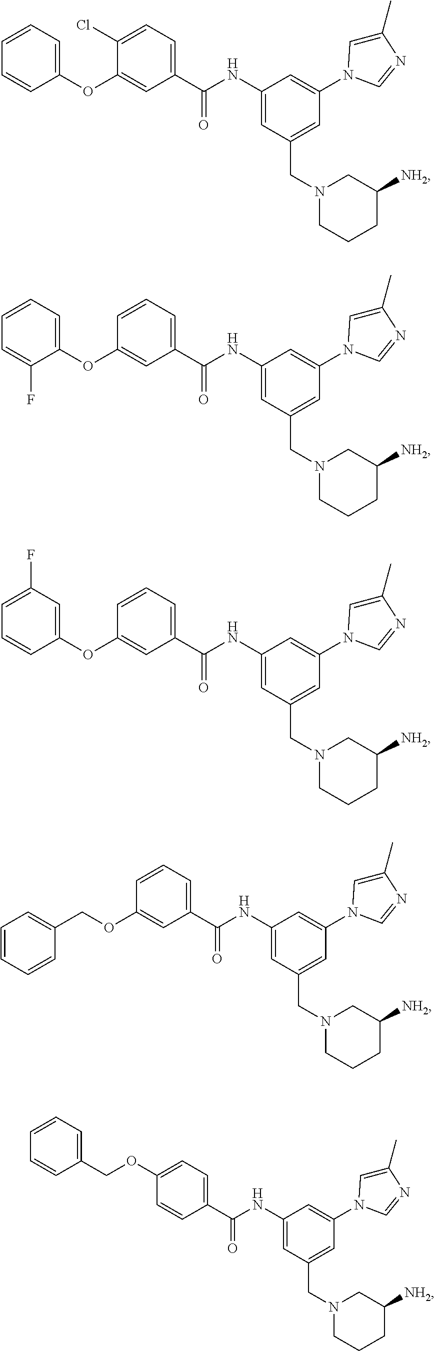 [SMILES: Cc1cn(-c2cc(CN3CCC[C@H](N)C3)cc(NC(=O)c3ccc(Cl)c(Oc4ccccc4)c3)c2)cn1 ; Cc1cn(-c2cc(CN3CCC[C@H](N)C3)cc(NC(=O)c3cccc(Oc4ccccc4F)c3)c2)cn1 ; Cc1cn(-c2cc(CN3CCC[C@H](N)C3)cc(NC(=O)c3cccc(Oc4cccc(F)c4)c3)c2)cn1 ; Cc1cn(-c2cc(CN3CCC[C@H](N)C3)cc(NC(=O)c3cccc(OCc4ccccc4)c3)c2)cn1 ; Cc1cn(-c2cc(CN3CCC[C@H](N)C3)cc(NC(=O)c3ccc(OCc4ccccc4)cc3)c2)cn1]

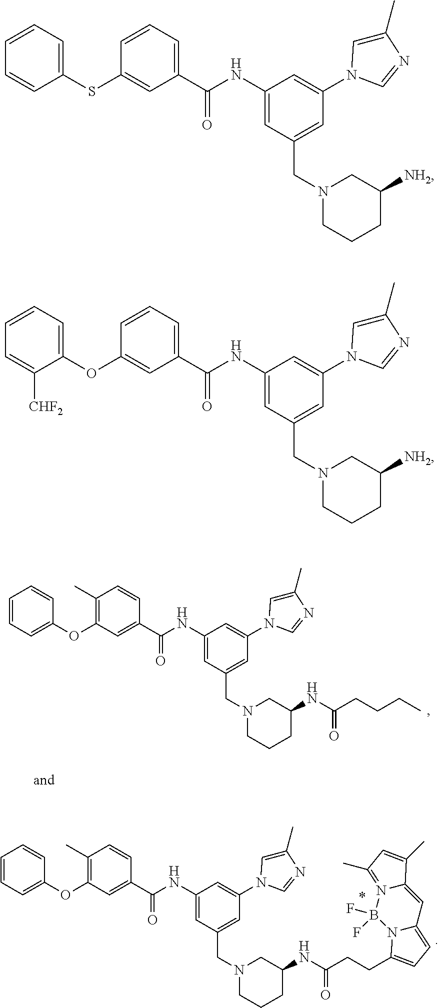

In particular embodiments of the invention, the compound of Formula (I) and/or Formula (III) has a structure selected from any one of the following in Table 2:

TABLE 2

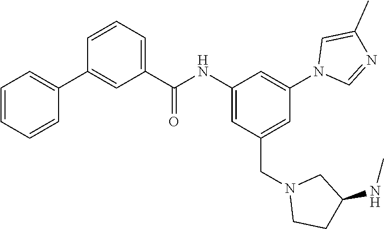 [SMILES: CN[C@H]1CCN(Cc2cc(NC(=O)c3cccc(-c4ccccc4)c3)cc(-n3cnc(C)c3)c2)C1]

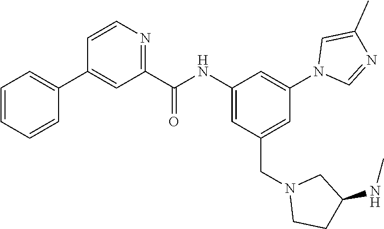 [SMILES: CN[C@H]1CCN(Cc2cc(NC(=O)c3cc(-c4ccccc4)ccn3)cc(-n3cnc(C)c3)c2)C1]

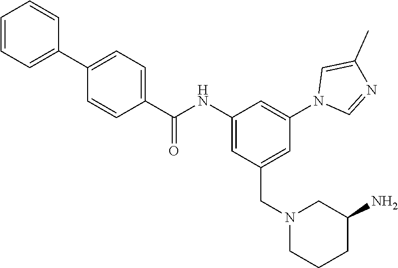 [SMILES: Cc1cn(-c2cc(CN3CCC[C@H](N)C3)cc(NC(=O)c3ccc(-c4ccccc4)cc3)c2)cn1]

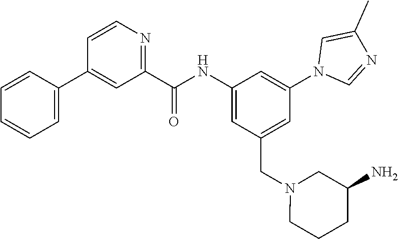 [SMILES: Cc1cn(-c2cc(CN3CCC[C@H](N)C3)cc(NC(=O)c3cc(-c4ccccc4)ccn3)c2)cn1]

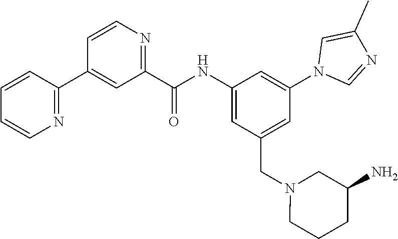 [SMILES: Cc1cn(-c2cc(CN3CCC[C@H](N)C3)cc(NC(=O)c3cc(-c4ccccn4)ccn3)c2)cn1]

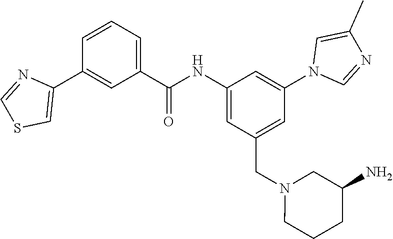 [SMILES: Cc1cn(-c2cc(CN3CCC[C@H](N)C3)cc(NC(=O)c3cccc(-c4cscn4)c3)c2)cn1]

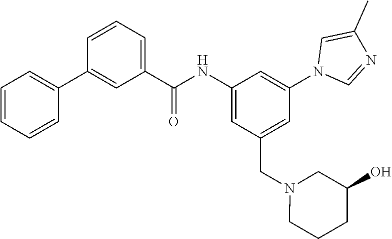 [SMILES: Cc1cn(-c2cc(CN3CCC[C@H](O)C3)cc(NC(=O)c3cccc(-c4ccccc4)c3)c2)cn1]

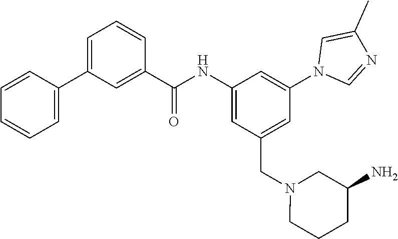 [SMILES: Cc1cn(-c2cc(CN3CCC[C@H](N)C3)cc(NC(=O)c3cccc(-c4ccccc4)c3)c2)cn1]

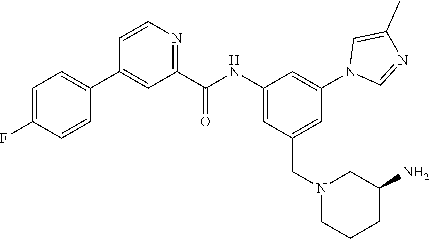 [SMILES: Cc1cn(-c2cc(CN3CCC[C@H](N)C3)cc(NC(=O)c3cc(-c4ccc(F)cc4)ccn3)c2)cn1]

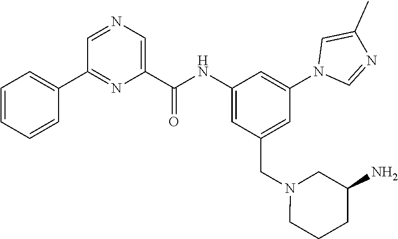 [SMILES: Cc1cn(-c2cc(CN3CCC[C@H](N)C3)cc(NC(=O)c3cncc(-c4ccccc4)n3)c2)cn1]

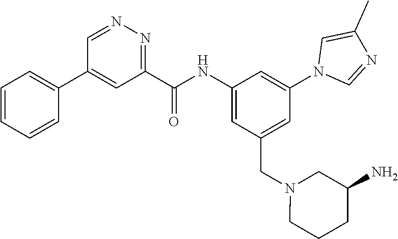 [SMILES: Cc1cn(-c2cc(CN3CCC[C@H](N)C3)cc(NC(=O)c3cc(-c4ccccc4)cnn3)c2)cn1]

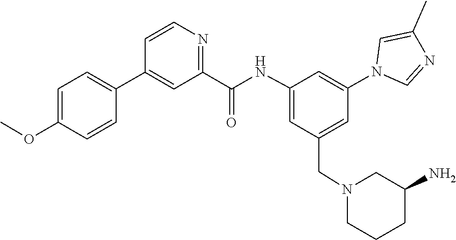 [SMILES: COc1ccc(-c2ccnc(C(=O)Nc3cc(CN4CCC[C@H](N)C4)cc(-n4cnc(C)c4)c3)c2)cc1]

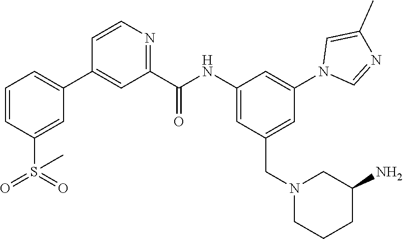 [SMILES: Cc1cn(-c2cc(CN3CCC[C@H](N)C3)cc(NC(=O)c3cc(-c4cccc(S(C)(=O)=O)c4)ccn3)c2)cn1]

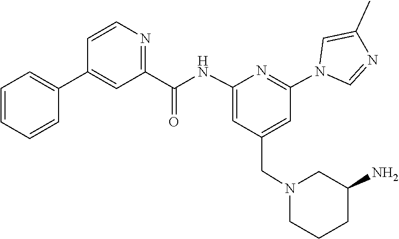 [SMILES: Cc1cn(-c2cc(CN3CCC[C@H](N)C3)cc(NC(=O)c3cc(-c4ccccc4)ccn3)n2)cn1]

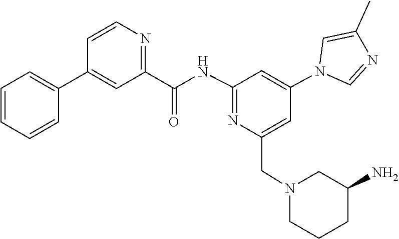 [SMILES: Cc1cn(-c2cc(CN3CCC[C@H](N)C3)nc(NC(=O)c3cc(-c4ccccc4)ccn3)c2)cn1]

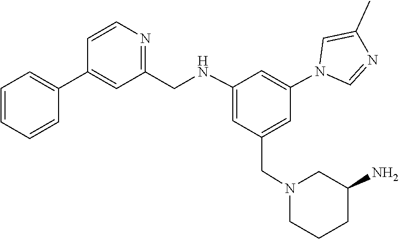 [SMILES: Cc1cn(-c2cc(CN3CCC[C@H](N)C3)cc(NCc3cc(-c4ccccc4)ccn3)c2)cn1]

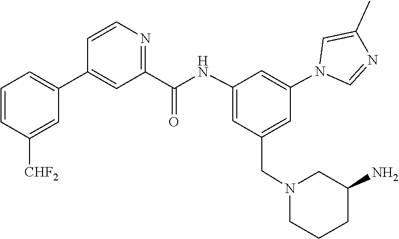 [SMILES: Cc1cn(-c2cc(CN3CCC[C@H](N)C3)cc(NC(=O)c3cc(-c4cccc(C(F)F)c4)ccn3)c2)cn1]

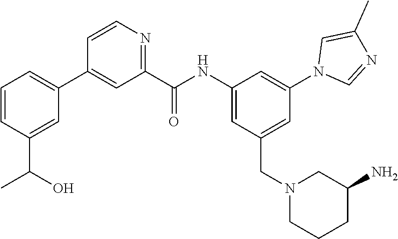 [SMILES: Cc1cn(-c2cc(CN3CCC[C@H](N)C3)cc(NC(=O)c3cc(-c4cccc(C(C)O)c4)ccn3)c2)cn1]

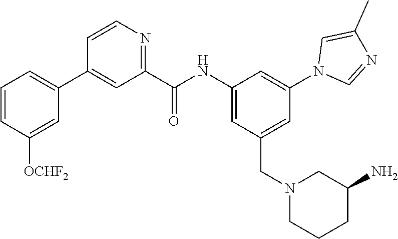 [SMILES: Cc1cn(-c2cc(CN3CCC[C@H](N)C3)cc(NC(=O)c3cc(-c4cccc(OC(F)F)c4)ccn3)c2)cn1]

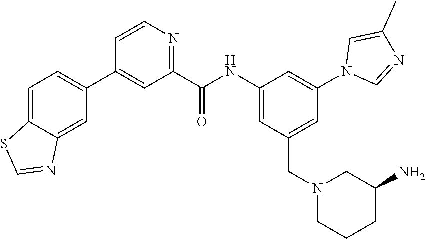 [SMILES: Cc1cn(-c2cc(CN3CCC[C@H](N)C3)cc(NC(=O)c3cc(-c4ccc5scnc5c4)ccn3)c2)cn1]

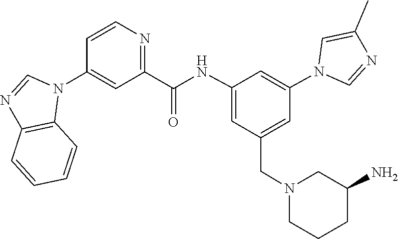 [SMILES: Cc1cn(-c2cc(CN3CCC[C@H](N)C3)cc(NC(=O)c3cc(-n4cnc5ccccc54)ccn3)c2)cn1]

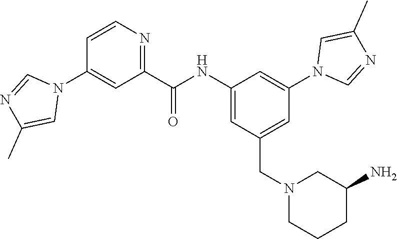 [SMILES: Cc1cn(-c2cc(CN3CCC[C@H](N)C3)cc(NC(=O)c3cc(-n4cnc(C)c4)ccn3)c2)cn1]

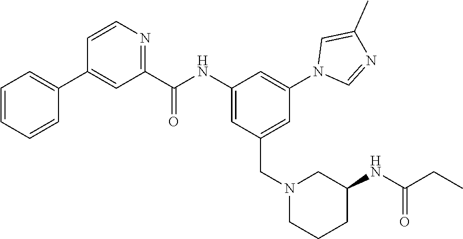 [SMILES: CCC(=O)N[C@H]1CCCN(Cc2cc(NC(=O)c3cc(-c4ccccc4)ccn3)cc(-n3cnc(C)c3)c2)C1]

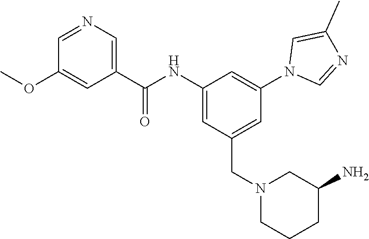 [SMILES: COc1cncc(C(=O)Nc2cc(CN3CCC[C@H](N)C3)cc(-n3cnc(C)c3)c2)c1]

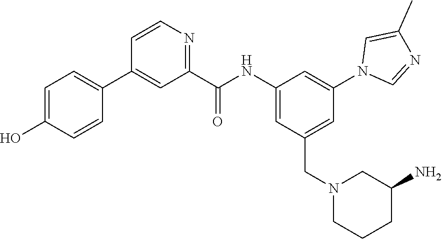 [SMILES: Cc1cn(-c2cc(CN3CCC[C@H](N)C3)cc(NC(=O)c3cc(-c4ccc(O)cc4)ccn3)c2)cn1]

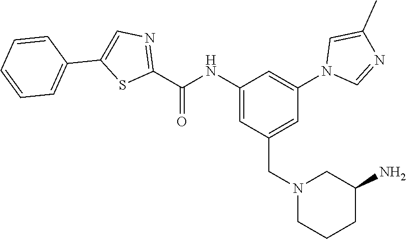 [SMILES: Cc1cn(-c2cc(CN3CCC[C@H](N)C3)cc(NC(=O)c3ncc(-c4ccccc4)s3)c2)cn1]

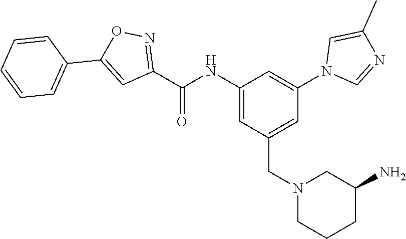 [SMILES: Cc1cn(-c2cc(CN3CCC[C@H](N)C3)cc(NC(=O)c3cc(-c4ccccc4)on3)c2)cn1]

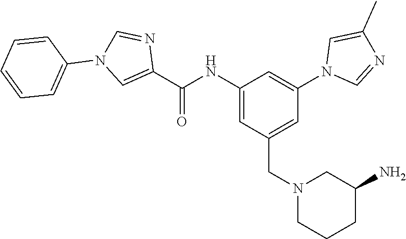 [SMILES: Cc1cn(-c2cc(CN3CCC[C@H](N)C3)cc(NC(=O)c3cn(-c4ccccc4)cn3)c2)cn1]

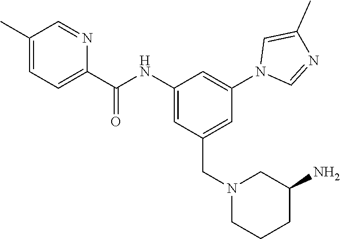 [SMILES: Cc1ccc(C(=O)Nc2cc(CN3CCC[C@H](N)C3)cc(-n3cnc(C)c3)c2)nc1]

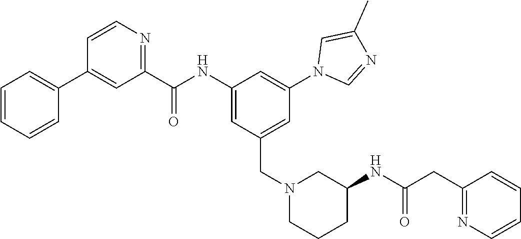 [SMILES: Cc1cn(-c2cc(CN3CCC[C@H](NC(=O)Cc4ccccn4)C3)cc(NC(=O)c3cc(-c4ccccc4)ccn3)c2)cn1]

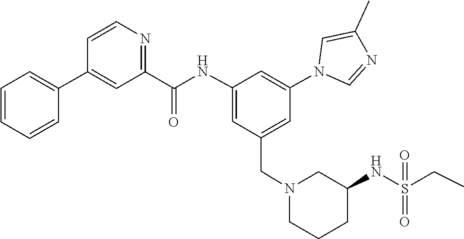 [SMILES: CCS(=O)(=O)N[C@H]1CCCN(Cc2cc(NC(=O)c3cc(-c4ccccc4)ccn3)cc(-n3cnc(C)c3)c2)C1]

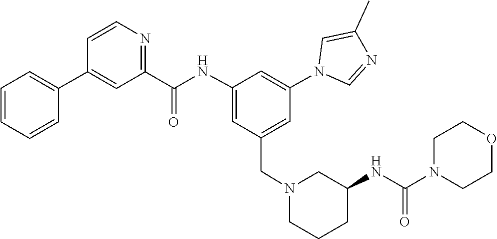 [SMILES: Cc1cn(-c2cc(CN3CCC[C@H](NC(=O)N4CCOCC4)C3)cc(NC(=O)c3cc(-c4ccccc4)ccn3)c2)cn1]

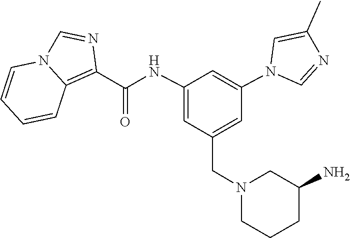 [SMILES: Cc1cn(-c2cc(CN3CCC[C@H](N)C3)cc(NC(=O)c3ncn4ccccc34)c2)cn1]

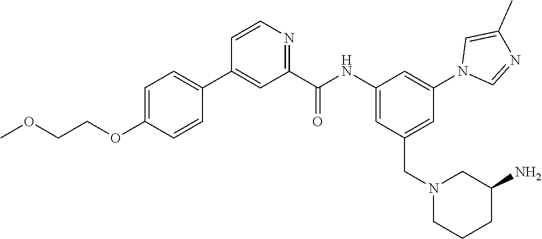 [SMILES: COCCOc1ccc(-c2ccnc(C(=O)Nc3cc(CN4CCC[C@H](N)C4)cc(-n4cnc(C)c4)c3)c2)cc1]

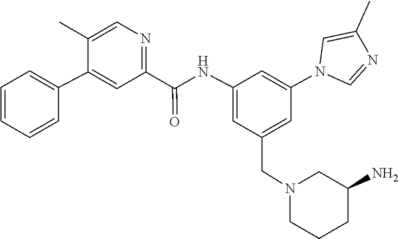 [SMILES: Cc1cn(-c2cc(CN3CCC[C@H](N)C3)cc(NC(=O)c3cc(-c4ccccc4)c(C)cn3)c2)cn1]

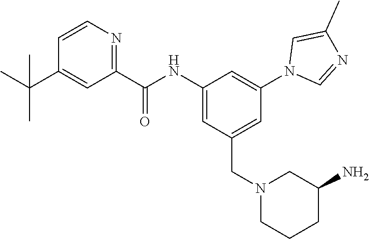 [SMILES: Cc1cn(-c2cc(CN3CCC[C@H](N)C3)cc(NC(=O)c3cc(C(C)(C)C)ccn3)c2)cn1]

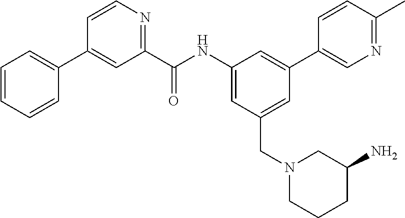 [SMILES: Cc1ccc(-c2cc(CN3CCC[C@H](N)C3)cc(NC(=O)c3cc(-c4ccccc4)ccn3)c2)cn1]

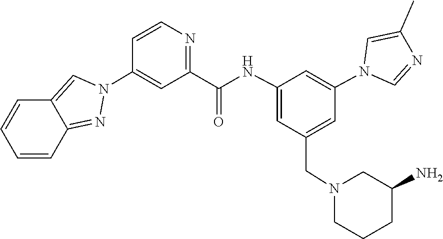 [SMILES: Cc1cn(-c2cc(CN3CCC[C@H](N)C3)cc(NC(=O)c3cc(-n4cc5ccccc5n4)ccn3)c2)cn1]

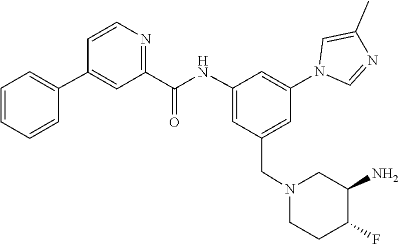 [SMILES: Cc1cn(-c2cc(CN3CC[C@@H](F)[C@H](N)C3)cc(NC(=O)c3cc(-c4ccccc4)ccn3)c2)cn1]

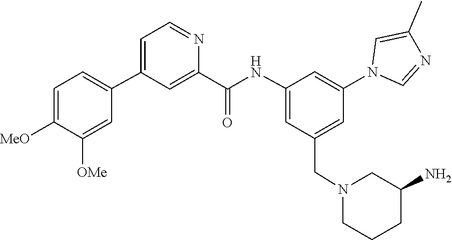 [SMILES: COc1ccc(-c2ccnc(C(=O)Nc3cc(CN4CCC[C@H](N)C4)cc(-n4cnc(C)c4)c3)c2)cc1OC]

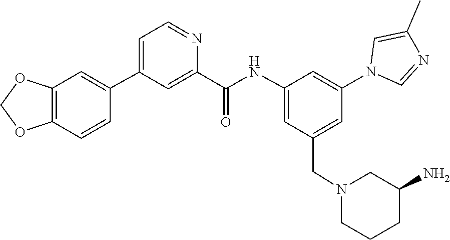 [SMILES: Cc1cn(-c2cc(CN3CCC[C@H](N)C3)cc(NC(=O)c3cc(-c4ccc5c(c4)OCO5)ccn3)c2)cn1]

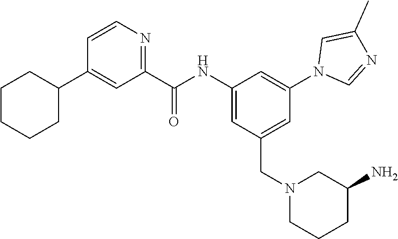 [SMILES: Cc1cn(-c2cc(CN3CCC[C@H](N)C3)cc(NC(=O)c3cc(C4CCCCC4)ccn3)c2)cn1]

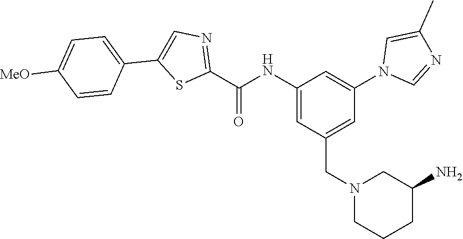 [SMILES: COc1ccc(-c2cnc(C(=O)Nc3cc(CN4CCC[C@H](N)C4)cc(-n4cnc(C)c4)c3)s2)cc1]

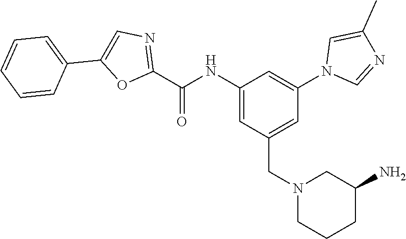 [SMILES: Cc1cn(-c2cc(CN3CCC[C@H](N)C3)cc(NC(=O)c3ncc(-c4ccccc4)o3)c2)cn1]

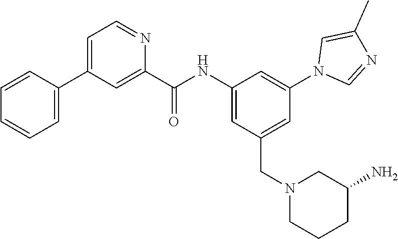 [SMILES: Cc1cn(-c2cc(CN3CCC[C@@H](N)C3)cc(NC(=O)c3cc(-c4ccccc4)ccn3)c2)cn1]

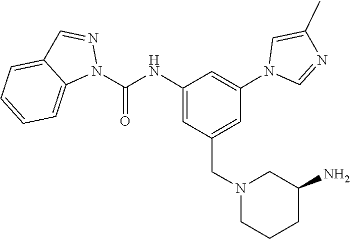 [SMILES: Cc1cn(-c2cc(CN3CCC[C@H](N)C3)cc(NC(=O)n3ncc4ccccc43)c2)cn1]

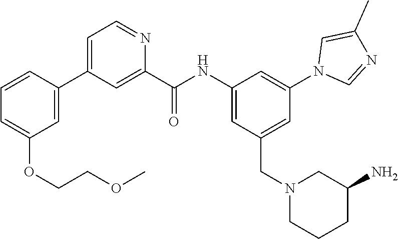 [SMILES: COCCOc1cccc(-c2ccnc(C(=O)Nc3cc(CN4CCC[C@H](N)C4)cc(-n4cnc(C)c4)c3)c2)c1]

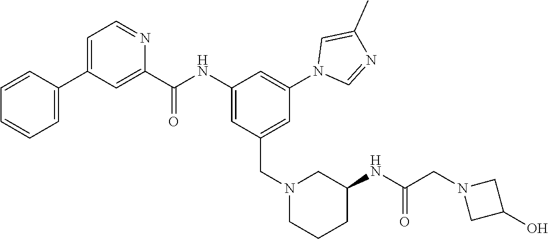 [SMILES: Cc1cn(-c2cc(CN3CCC[C@H](NC(=O)CN4CC(O)C4)C3)cc(NC(=O)c3cc(-c4ccccc4)ccn3)c2)cn1]

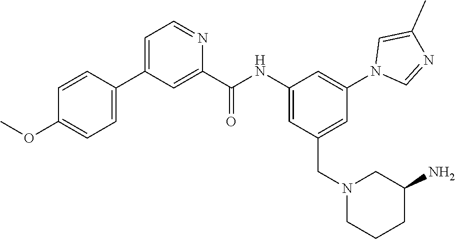 [SMILES: COc1ccc(-c2ccnc(C(=O)Nc3cc(CN4CCC[C@H](N)C4)cc(-n4cnc(C)c4)c3)c2)cc1]

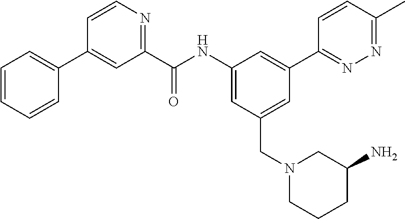 [SMILES: Cc1ccc(-c2cc(CN3CCC[C@H](N)C3)cc(NC(=O)c3cc(-c4ccccc4)ccn3)c2)nn1]

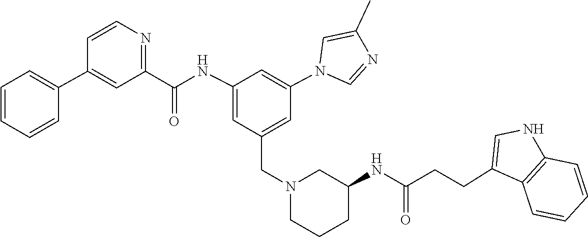 [SMILES: Cc1cn(-c2cc(CN3CCC[C@H](NC(=O)CCc4c[nH]c5ccccc45)C3)cc(NC(=O)c3cc(-c4ccccc4)ccn3)c2)cn1]

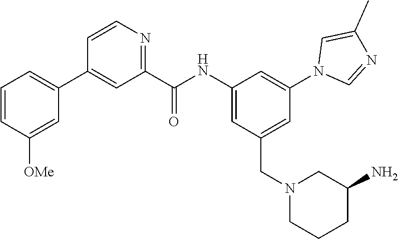 [SMILES: COc1cccc(-c2ccnc(C(=O)Nc3cc(CN4CCC[C@H](N)C4)cc(-n4cnc(C)c4)c3)c2)c1]

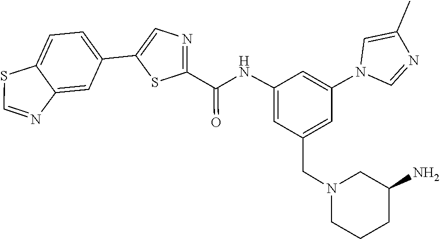 [SMILES: Cc1cn(-c2cc(CN3CCC[C@H](N)C3)cc(NC(=O)c3ncc(-c4ccc5scnc5c4)s3)c2)cn1]

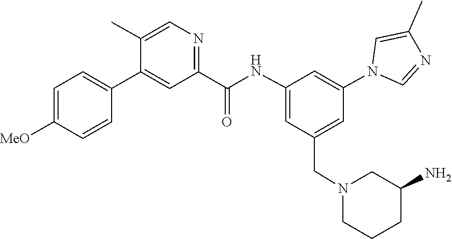 [SMILES: COc1ccc(-c2cc(C(=O)Nc3cc(CN4CCC[C@H](N)C4)cc(-n4cnc(C)c4)c3)ncc2C)cc1]

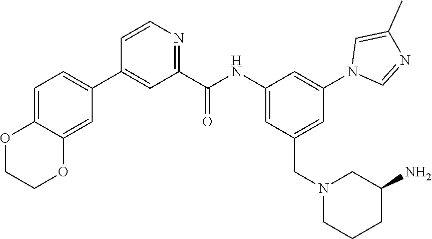 [SMILES: Cc1cn(-c2cc(CN3CCC[C@H](N)C3)cc(NC(=O)c3cc(-c4ccc5c(c4)OCCO5)ccn3)c2)cn1]

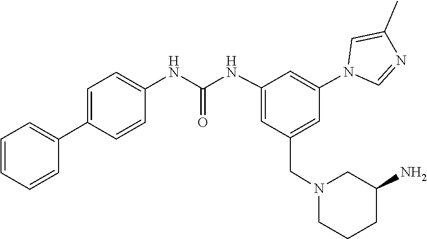 [SMILES: Cc1cn(-c2cc(CN3CCC[C@H](N)C3)cc(NC(=O)Nc3ccc(-c4ccccc4)cc3)c2)cn1]

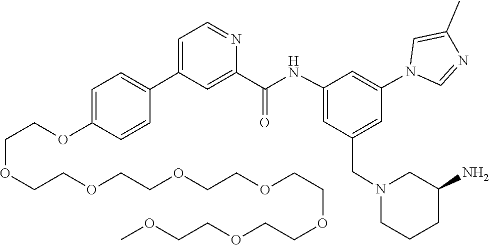 [SMILES: COCCOCCOCCOCCOCCOCCOCCOc1ccc(-c2ccnc(C(=O)Nc3cc(CN4CCC[C@H](N)C4)cc(-n4cnc(C)c4)c3)c2)cc1]

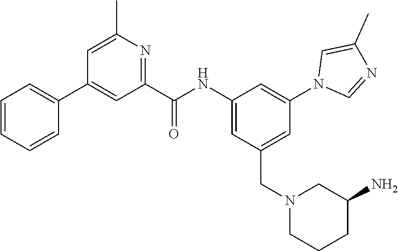 [SMILES: Cc1cn(-c2cc(CN3CCC[C@H](N)C3)cc(NC(=O)c3cc(-c4ccccc4)cc(C)n3)c2)cn1]

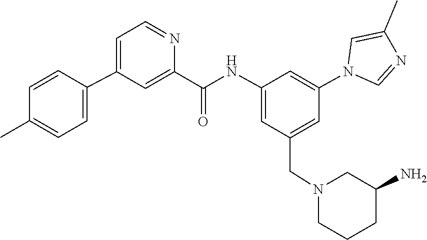 [SMILES: Cc1ccc(-c2ccnc(C(=O)Nc3cc(CN4CCC[C@H](N)C4)cc(-n4cnc(C)c4)c3)c2)cc1]

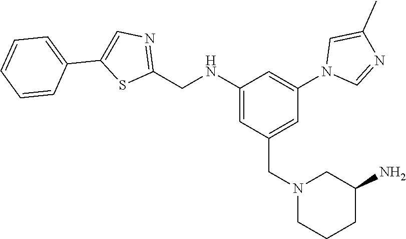 [SMILES: Cc1cn(-c2cc(CN3CCC[C@H](N)C3)cc(NCc3ncc(-c4ccccc4)s3)c2)cn1]

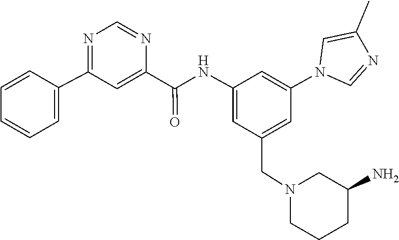 [SMILES: Cc1cn(-c2cc(CN3CCC[C@H](N)C3)cc(NC(=O)c3cc(-c4ccccc4)ncn3)c2)cn1]

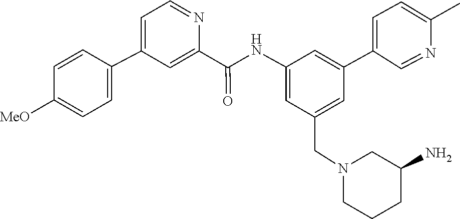 [SMILES: COc1ccc(-c2ccnc(C(=O)Nc3cc(CN4CCC[C@H](N)C4)cc(-c4ccc(C)nc4)c3)c2)cc1]

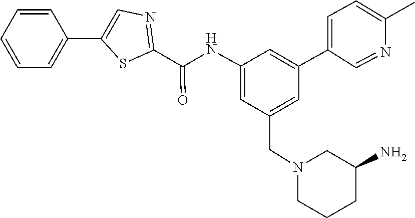 [SMILES: Cc1ccc(-c2cc(CN3CCC[C@H](N)C3)cc(NC(=O)c3ncc(-c4ccccc4)s3)c2)cn1]

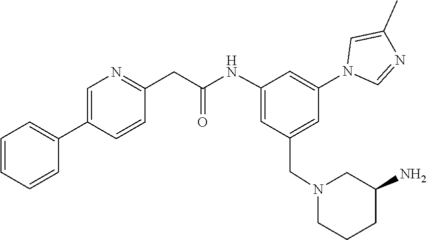 [SMILES: Cc1cn(-c2cc(CN3CCC[C@H](N)C3)cc(NC(=O)Cc3ccc(-c4ccccc4)cn3)c2)cn1]

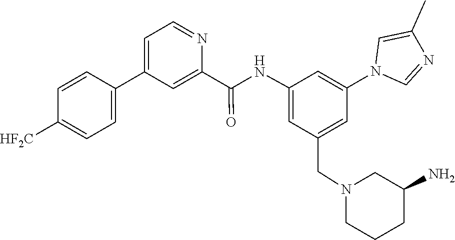

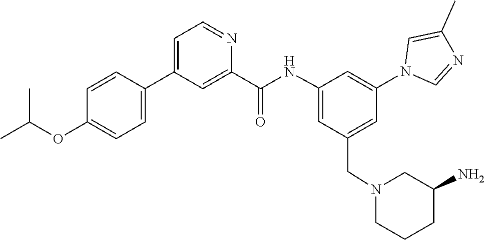 [SMILES: Cc1cn(-c2cc(CN3CCC[C@H](N)C3)cc(NC(=O)c3cc(-c4ccc(OC(C)C)cc4)ccn3)c2)cn1]

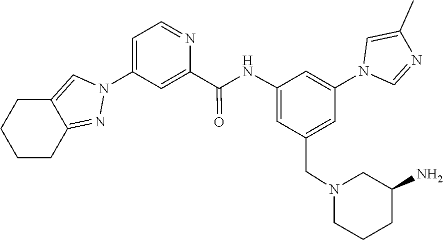 [SMILES: Cc1cn(-c2cc(CN3CCC[C@H](N)C3)cc(NC(=O)c3cc(-n4cc5c(n4)CCCC5)ccn3)c2)cn1]

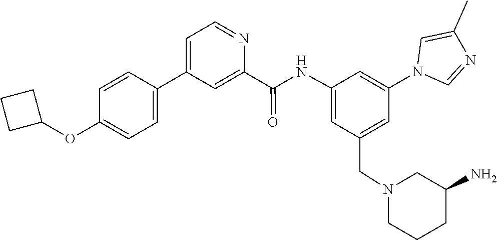 [SMILES: Cc1cn(-c2cc(CN3CCC[C@H](N)C3)cc(NC(=O)c3cc(-c4ccc(OC5CCC5)cc4)ccn3)c2)cn1]

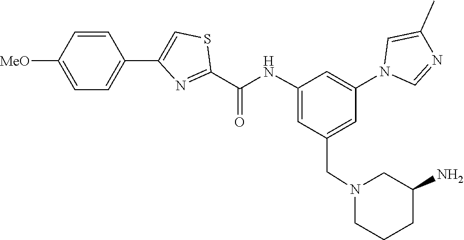 [SMILES: COc1ccc(-c2csc(C(=O)Nc3cc(CN4CCC[C@H](N)C4)cc(-n4cnc(C)c4)c3)n2)cc1]

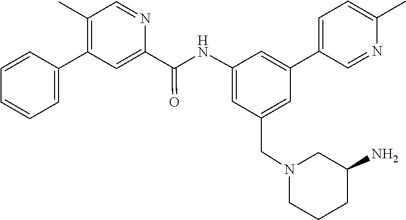 [SMILES: Cc1ccc(-c2cc(CN3CCC[C@H](N)C3)cc(NC(=O)c3cc(-c4ccccc4)c(C)cn3)c2)cn1]

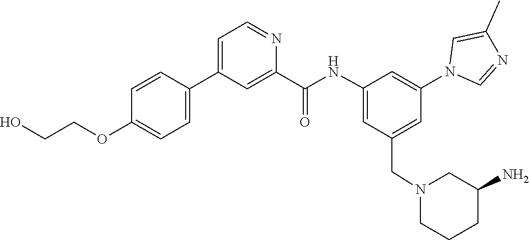 [SMILES: Cc1cn(-c2cc(CN3CCC[C@H](N)C3)cc(NC(=O)c3cc(-c4ccc(OCCO)cc4)ccn3)c2)cn1]

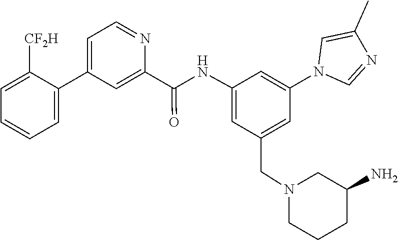 [SMILES: Cc1cn(-c2cc(CN3CCC[C@H](N)C3)cc(NC(=O)c3cc(-c4ccccc4C(F)F)ccn3)c2)cn1]

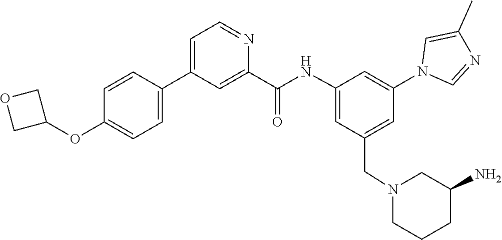 [SMILES: Cc1cn(-c2cc(CN3CCC[C@H](N)C3)cc(NC(=O)c3cc(-c4ccc(OC5COC5)cc4)ccn3)c2)cn1]

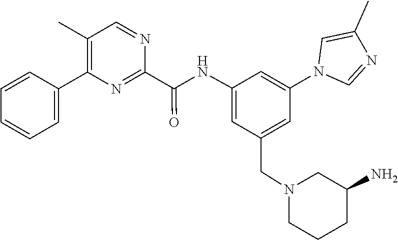 [SMILES: Cc1cn(-c2cc(CN3CCC[C@H](N)C3)cc(NC(=O)c3ncc(C)c(-c4ccccc4)n3)c2)cn1]

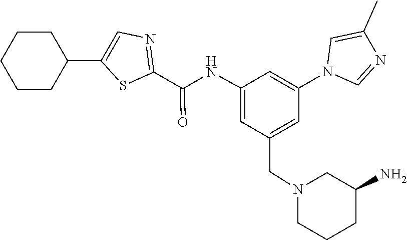 [SMILES: Cc1cn(-c2cc(CN3CCC[C@H](N)C3)cc(NC(=O)c3ncc(C4CCCCC4)s3)c2)cn1]

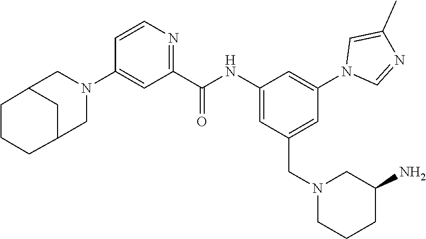 [SMILES: Cc1cn(-c2cc(CN3CCC[C@H](N)C3)cc(NC(=O)c3cc(N4CC5CCCC(C5)C4)ccn3)c2)cn1]

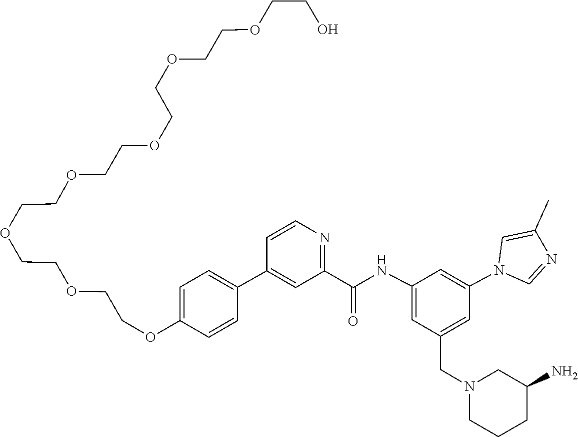 [SMILES: Cc1cn(-c2cc(CN3CCC[C@H](N)C3)cc(NC(=O)c3cc(-c4ccc(OCCOCCOCCOCCOCCOCCOCCO)cc4)ccn3)c2)cn1]

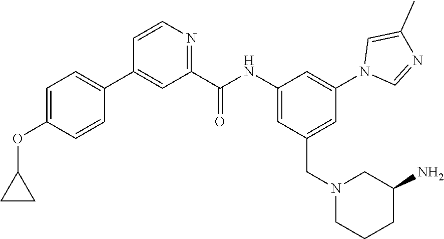 [SMILES: Cc1cn(-c2cc(CN3CCC[C@H](N)C3)cc(NC(=O)c3cc(-c4ccc(OC5CC5)cc4)ccn3)c2)cn1]

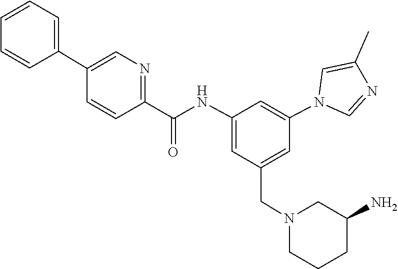 [SMILES: Cc1cn(-c2cc(CN3CCC[C@H](N)C3)cc(NC(=O)c3ccc(-c4ccccc4)cn3)c2)cn1]

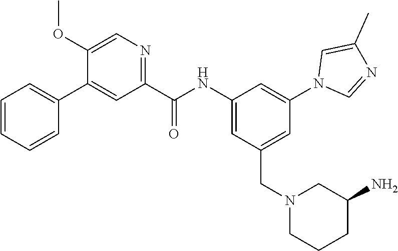 [SMILES: COc1cnc(C(=O)Nc2cc(CN3CCC[C@H](N)C3)cc(-n3cnc(C)c3)c2)cc1-c1ccccc1]

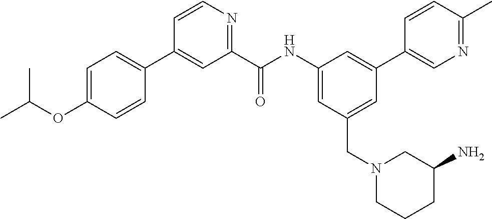 [SMILES: Cc1ccc(-c2cc(CN3CCC[C@H](N)C3)cc(NC(=O)c3cc(-c4ccc(OC(C)C)cc4)ccn3)c2)cn1]

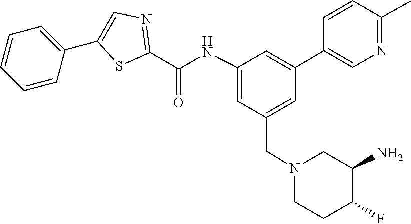 [SMILES: Cc1ccc(-c2cc(CN3CC[C@@H](F)[C@H](N)C3)cc(NC(=O)c3ncc(-c4ccccc4)s3)c2)cn1]

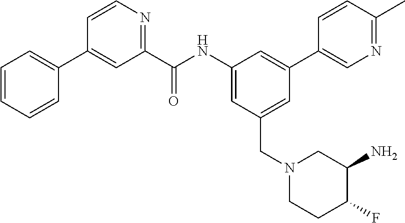 [SMILES: Cc1ccc(-c2cc(CN3CC[C@@H](F)[C@H](N)C3)cc(NC(=O)c3cc(-c4ccccc4)ccn3)c2)cn1]

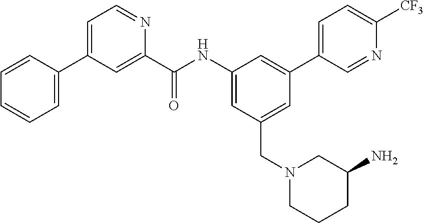 [SMILES: N[C@H]1CCCN(Cc2cc(NC(=O)c3cc(-c4ccccc4)ccn3)cc(-c3ccc(C(F)(F)F)nc3)c2)C1]

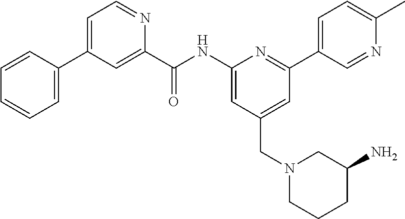 [SMILES: Cc1ccc(-c2cc(CN3CCC[C@H](N)C3)cc(NC(=O)c3cc(-c4ccccc4)ccn3)n2)cn1]

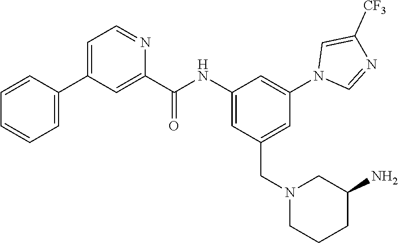 [SMILES: N[C@H]1CCCN(Cc2cc(NC(=O)c3cc(-c4ccccc4)ccn3)cc(-n3cnc(C(F)(F)F)c3)c2)C1]

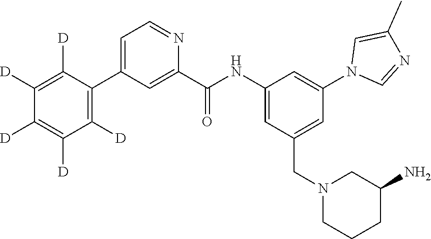 [SMILES: [2H]c1c([2H])c([2H])c(-c2ccnc(C(=O)Nc3cc(CN4CCC[C@H](N)C4)cc(-n4cnc(C)c4)c3)c2)c([2H])c1[2H]]

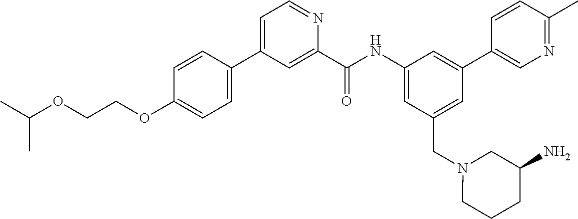 [SMILES: Cc1ccc(-c2cc(CN3CCC[C@H](N)C3)cc(NC(=O)c3cc(-c4ccc(OCCOC(C)C)cc4)ccn3)c2)cn1]

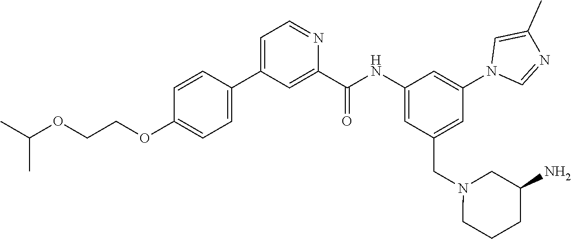 [SMILES: Cc1cn(-c2cc(CN3CCC[C@H](N)C3)cc(NC(=O)c3cc(-c4ccc(OCCOC(C)C)cc4)ccn3)c2)cn1]

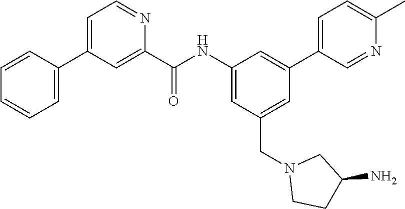 [SMILES: Cc1ccc(-c2cc(CN3CC[C@H](N)C3)cc(NC(=O)c3cc(-c4ccccc4)ccn3)c2)cn1]

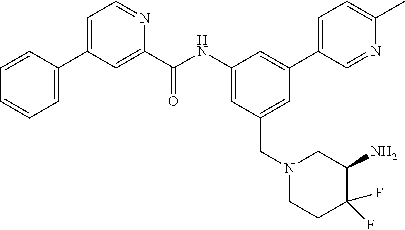 [SMILES: Cc1ccc(-c2cc(CN3CCC(F)(F)[C@H](N)C3)cc(NC(=O)c3cc(-c4ccccc4)ccn3)c2)cn1]

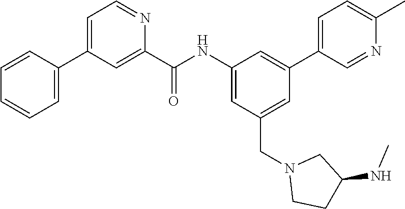 [SMILES: CN[C@H]1CCN(Cc2cc(NC(=O)c3cc(-c4ccccc4)ccn3)cc(-c3ccc(C)nc3)c2)C1]

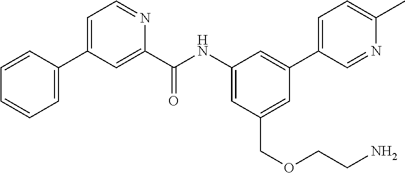 [SMILES: Cc1ccc(-c2cc(COCCN)cc(NC(=O)c3cc(-c4ccccc4)ccn3)c2)cn1]

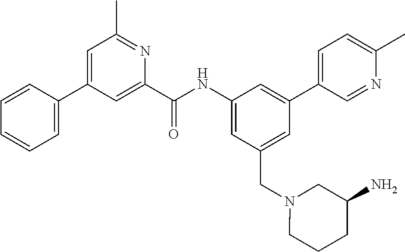 [SMILES: Cc1ccc(-c2cc(CN3CCC[C@H](N)C3)cc(NC(=O)c3cc(-c4ccccc4)cc(C)n3)c2)cn1]

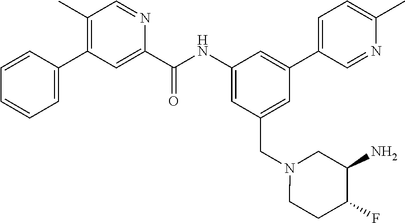 [SMILES: Cc1ccc(-c2cc(CN3CC[C@@H](F)[C@H](N)C3)cc(NC(=O)c3cc(-c4ccccc4)c(C)cn3)c2)cn1]

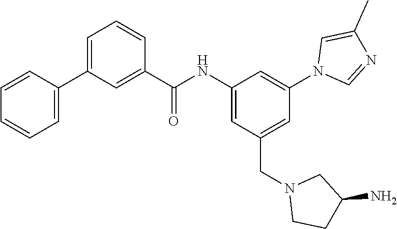 [SMILES: Cc1cn(-c2cc(CN3CC[C@H](N)C3)cc(NC(=O)c3cccc(-c4ccccc4)c3)c2)cn1]

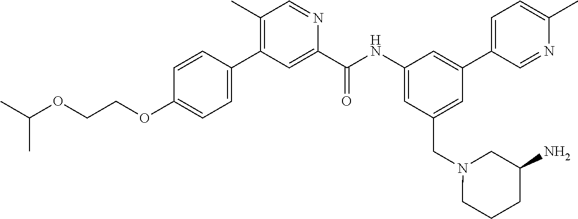 [SMILES: Cc1ccc(-c2cc(CN3CCC[C@H](N)C3)cc(NC(=O)c3cc(-c4ccc(OCCOC(C)C)cc4)c(C)cn3)c2)cn1]

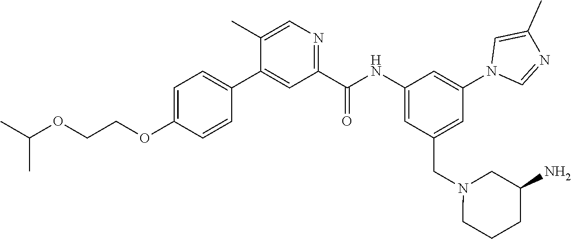 [SMILES: Cc1cn(-c2cc(CN3CCC[C@H](N)C3)cc(NC(=O)c3cc(-c4ccc(OCCOC(C)C)cc4)c(C)cn3)c2)cn1]

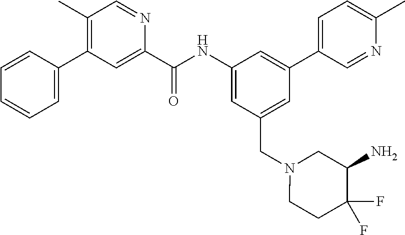 [SMILES: Cc1ccc(-c2cc(CN3CCC(F)(F)[C@H](N)C3)cc(NC(=O)c3cc(-c4ccccc4)c(C)cn3)c2)cn1]

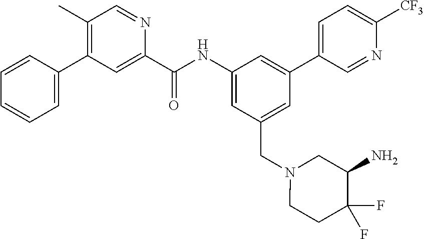 [SMILES: Cc1cnc(C(=O)Nc2cc(CN3CCC(F)(F)[C@H](N)C3)cc(-c3ccc(C(F)(F)F)nc3)c2)cc1-c1ccccc1]

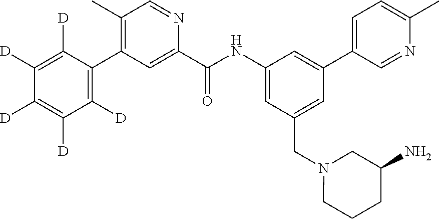 [SMILES: [2H]c1c([2H])c([2H])c(-c2cc(C(=O)Nc3cc(CN4CCC[C@H](N)C4)cc(-c4ccc(C)nc4)c3)ncc2C)c([2H])c1[2H]]

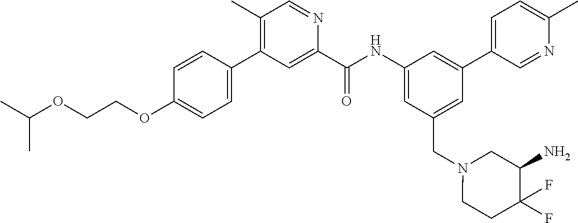 [SMILES: Cc1ccc(-c2cc(CN3CCC(F)(F)[C@H](N)C3)cc(NC(=O)c3cc(-c4ccc(OCCOC(C)C)cc4)c(C)cn3)c2)cn1]

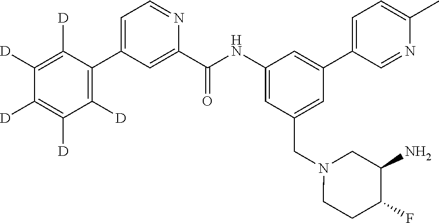 [SMILES: [2H]c1c([2H])c([2H])c(-c2ccnc(C(=O)Nc3cc(CN4CC[C@@H](F)[C@H](N)C4)cc(-c4ccc(C)nc4)c3)c2)c([2H])c1[2H]]

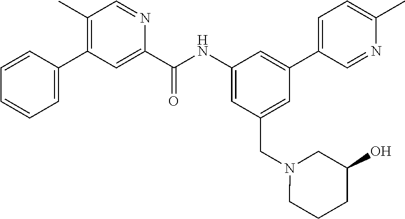 [SMILES: Cc1ccc(-c2cc(CN3CCC[C@H](O)C3)cc(NC(=O)c3cc(-c4ccccc4)c(C)cn3)c2)cn1]

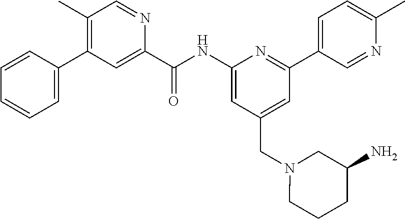 [SMILES: Cc1ccc(-c2cc(CN3CCC[C@H](N)C3)cc(NC(=O)c3cc(-c4ccccc4)c(C)cn3)n2)cn1]

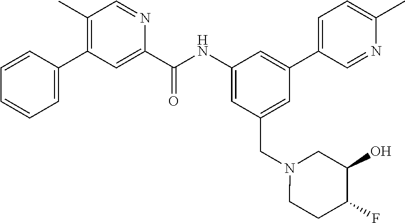 [SMILES: Cc1ccc(-c2cc(CN3CC[C@@H](F)[C@H](O)C3)cc(NC(=O)c3cc(-c4ccccc4)c(C)cn3)c2)cn1]

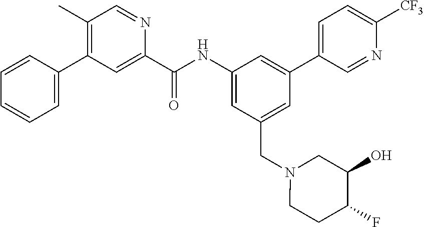 [SMILES: Cc1cnc(C(=O)Nc2cc(CN3CC[C@@H](F)[C@H](O)C3)cc(-c3ccc(C(F)(F)F)nc3)c2)cc1-c1ccccc1]

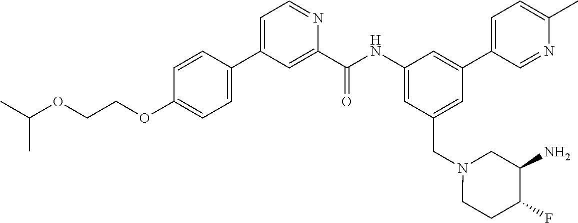 [SMILES: Cc1ccc(-c2cc(CN3CC[C@@H](F)[C@H](N)C3)cc(NC(=O)c3cc(-c4ccc(OCCOC(C)C)cc4)ccn3)c2)cn1]

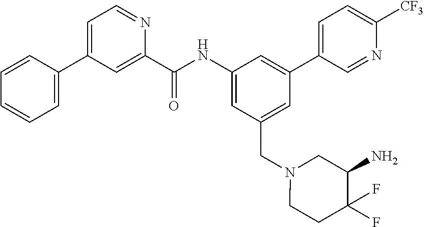 [SMILES: N[C@@H]1CN(Cc2cc(NC(=O)c3cc(-c4ccccc4)ccn3)cc(-c3ccc(C(F)(F)F)nc3)c2)CCC1(F)F]

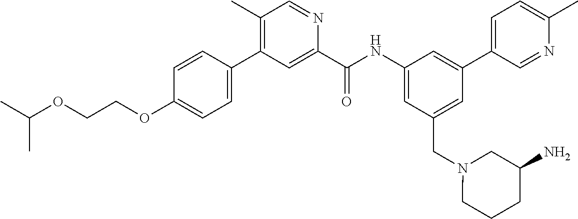 [SMILES: Cc1ccc(-c2cc(CN3CCC[C@H](N)C3)cc(NC(=O)c3cc(-c4ccc(OCCOC(C)C)cc4)c(C)cn3)c2)cn1]

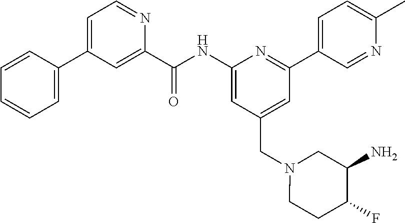 [SMILES: Cc1ccc(-c2cc(CN3CC[C@@H](F)[C@H](N)C3)cc(NC(=O)c3cc(-c4ccccc4)ccn3)n2)cn1]

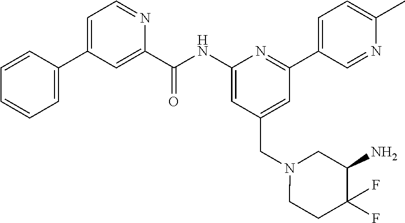 [SMILES: Cc1ccc(-c2cc(CN3CCC(F)(F)[C@H](N)C3)cc(NC(=O)c3cc(-c4ccccc4)ccn3)n2)cn1]

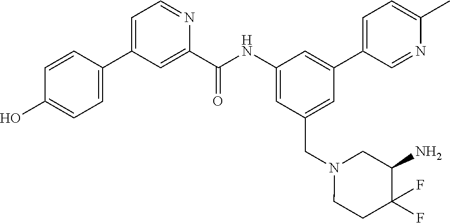 [SMILES: Cc1ccc(-c2cc(CN3CCC(F)(F)[C@H](N)C3)cc(NC(=O)c3cc(-c4ccc(O)cc4)ccn3)c2)cn1]

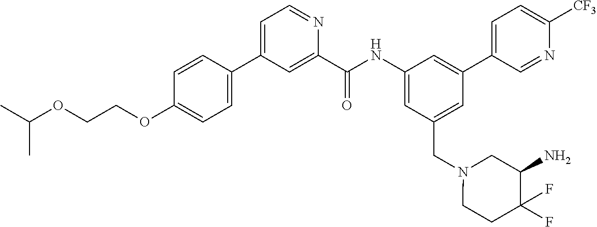 [SMILES: CC(C)OCCOc1ccc(-c2ccnc(C(=O)Nc3cc(CN4CCC(F)(F)[C@H](N)C4)cc(-c4ccc(C(F)(F)F)nc4)c3)c2)cc1]

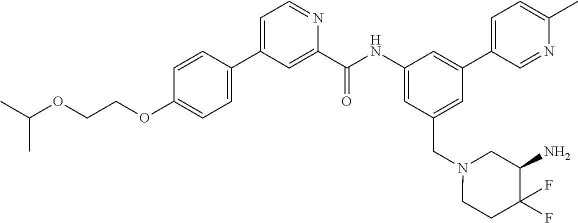 [SMILES: Cc1ccc(-c2cc(CN3CCC(F)(F)[C@H](N)C3)cc(NC(=O)c3cc(-c4ccc(OCCOC(C)C)cc4)ccn3)c2)cn1]

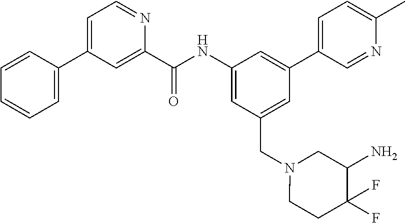 [SMILES: Cc1ccc(-c2cc(CN3CCC(F)(F)C(N)C3)cc(NC(=O)c3cc(-c4ccccc4)ccn3)c2)cn1]

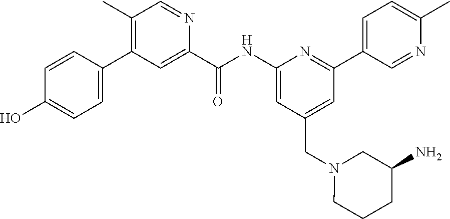 [SMILES: Cc1ccc(-c2cc(CN3CCC[C@H](N)C3)cc(NC(=O)c3cc(-c4ccc(O)cc4)c(C)cn3)n2)cn1]

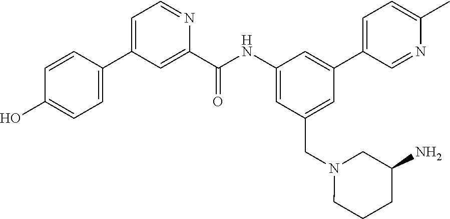 [SMILES: Cc1ccc(-c2cc(CN3CCC[C@H](N)C3)cc(NC(=O)c3cc(-c4ccc(O)cc4)ccn3)c2)cn1]

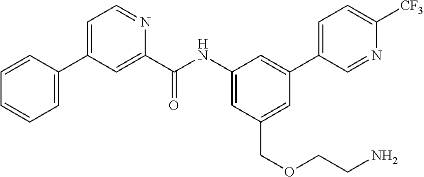 [SMILES: NCCOCc1cc(NC(=O)c2cc(-c3ccccc3)ccn2)cc(-c2ccc(C(F)(F)F)nc2)c1]

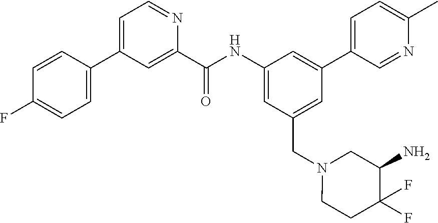 [SMILES: Cc1ccc(-c2cc(CN3CCC(F)(F)[C@H](N)C3)cc(NC(=O)c3cc(-c4ccc(F)cc4)ccn3)c2)cn1]

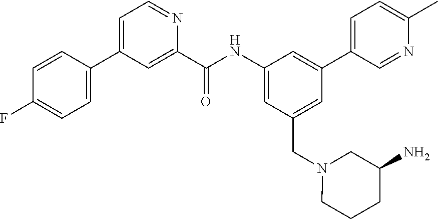 [SMILES: Cc1ccc(-c2cc(CN3CCC[C@H](N)C3)cc(NC(=O)c3cc(-c4ccc(F)cc4)ccn3)c2)cn1]

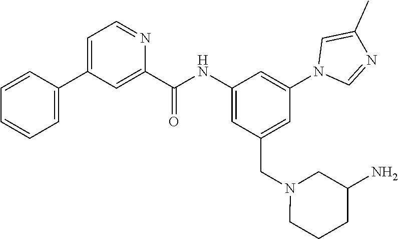 [SMILES: Cc1cn(-c2cc(CN3CCCC(N)C3)cc(NC(=O)c3cc(-c4ccccc4)ccn3)c2)cn1]

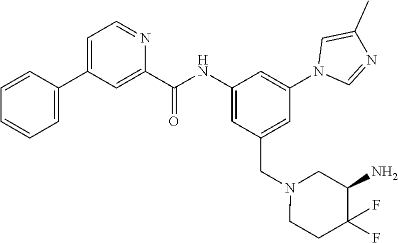 [SMILES: Cc1cn(-c2cc(CN3CCC(F)(F)[C@H](N)C3)cc(NC(=O)c3cc(-c4ccccc4)ccn3)c2)cn1]

In a particularly preferred embodiment, the compound has the structure

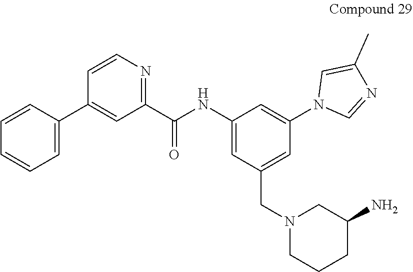 [SMILES: Cc1cn(-c2cc(CN3CCC[C@H](N)C3)cc(NC(=O)c3cc(-c4ccccc4)ccn3)c2)cn1]

In particular embodiments of the invention, the compound of Formula (I) or Formula (II) or Formula (III) or Formula (IIIa) or Formula (IIIb) or Formula (IVa) or Formula (IVb) or Formula (V) or Formula (VIa) or Formula (VIb) has a structure selected from any one of the following in Table 3:

TABLE 3

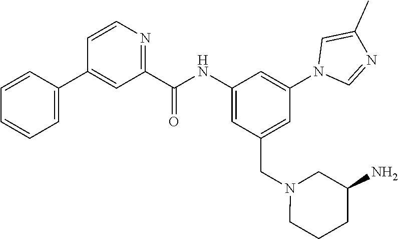 [SMILES: Cc1cn(-c2cc(CN3CCC[C@H](N)C3)cc(NC(=O)c3cc(-c4ccccc4)ccn3)c2)cn1]

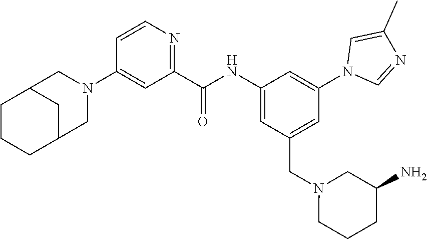 [SMILES: Cc1cn(-c2cc(CN3CCC[C@H](N)C3)cc(NC(=O)c3cc(N4CC5CCCC(C5)C4)ccn3)c2)cn1]

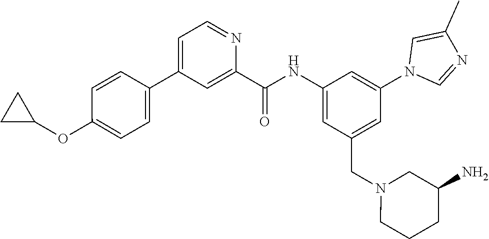 [SMILES: Cc1cn(-c2cc(CN3CCC[C@H](N)C3)cc(NC(=O)c3cc(-c4ccc(OC5CC5)cc4)ccn3)c2)cn1]

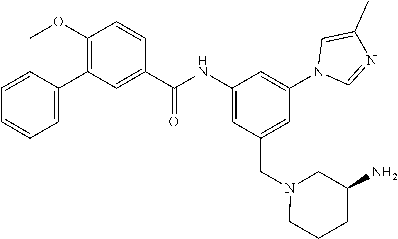 [SMILES: COc1ccc(C(=O)Nc2cc(CN3CCC[C@H](N)C3)cc(-n3cnc(C)c3)c2)cc1-c1ccccc1]

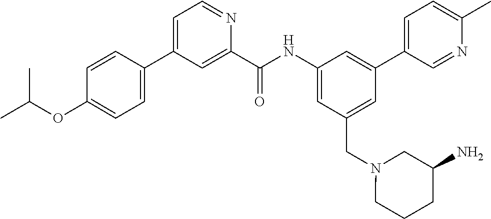 [SMILES: Cc1ccc(-c2cc(CN3CCC[C@H](N)C3)cc(NC(=O)c3cc(-c4ccc(OC(C)C)cc4)ccn3)c2)cn1]

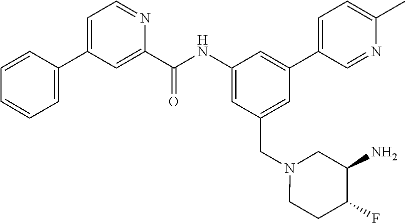 [SMILES: Cc1ccc(-c2cc(CN3CC[C@@H](F)[C@H](N)C3)cc(NC(=O)c3cc(-c4ccccc4)ccn3)c2)cn1]

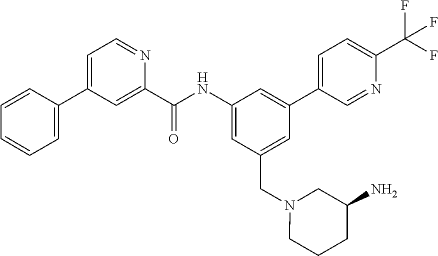 [SMILES: N[C@H]1CCCN(Cc2cc(NC(=O)c3cc(-c4ccccc4)ccn3)cc(-c3ccc(C(F)(F)F)nc3)c2)C1]

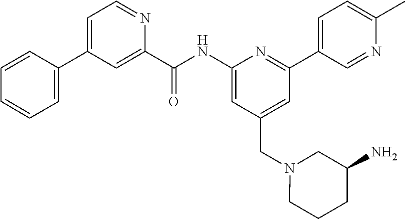 [SMILES: Cc1ccc(-c2cc(CN3CCC[C@H](N)C3)cc(NC(=O)c3cc(-c4ccccc4)ccn3)n2)cn1]

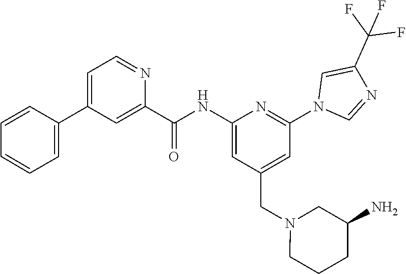 [SMILES: N[C@H]1CCCN(Cc2cc(NC(=O)c3cc(-c4ccccc4)ccn3)nc(-n3cnc(C(F)(F)F)c3)c2)C1]

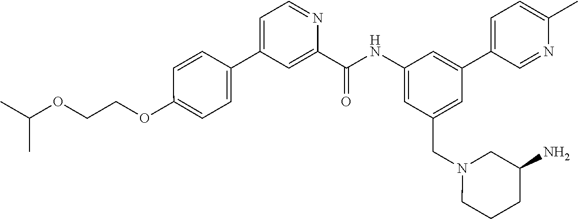 [SMILES: Cc1ccc(-c2cc(CN3CCC[C@H](N)C3)cc(NC(=O)c3cc(-c4ccc(OCCOC(C)C)cc4)ccn3)c2)cn1]

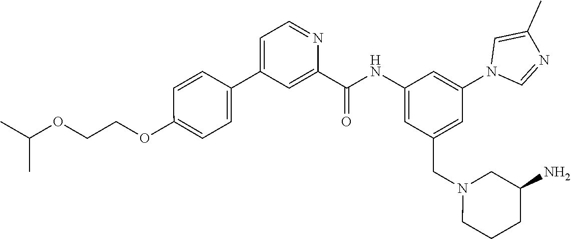 [SMILES: Cc1cn(-c2cc(CN3CCC[C@H](N)C3)cc(NC(=O)c3cc(-c4ccc(OCCOC(C)C)cc4)ccn3)c2)cn1]

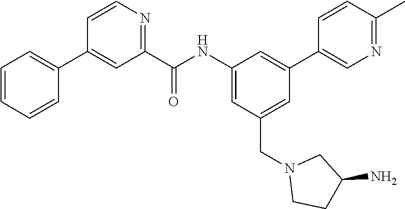 [SMILES: Cc1ccc(-c2cc(CN3CC[C@H](N)C3)cc(NC(=O)c3cc(-c4ccccc4)ccn3)c2)cn1]

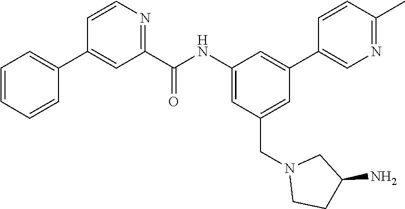 [SMILES: Cc1ccc(-c2cc(CN3CC[C@H](N)C3)cc(NC(=O)c3cc(-c4ccccc4)ccn3)c2)cn1]

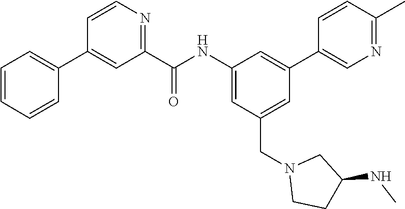 [SMILES: CN[C@H]1CCN(Cc2cc(NC(=O)c3cc(-c4ccccc4)ccn3)cc(-c3ccc(C)nc3)c2)C1]

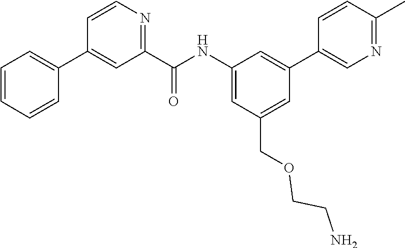 [SMILES: Cc1ccc(-c2cc(COCCN)cc(NC(=O)c3cc(-c4ccccc4)ccn3)c2)cn1]

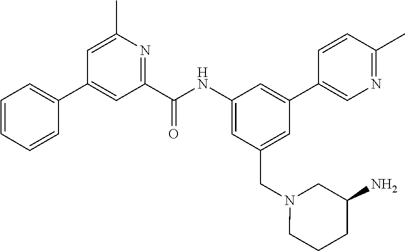 [SMILES: Cc1ccc(-c2cc(CN3CCC[C@H](N)C3)cc(NC(=O)c3cc(-c4ccccc4)cc(C)n3)c2)cn1]

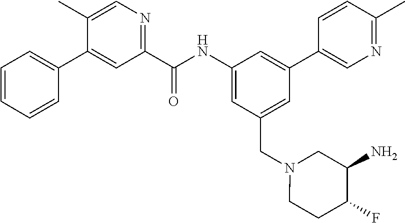 [SMILES: Cc1ccc(-c2cc(CN3CC[C@@H](F)[C@H](N)C3)cc(NC(=O)c3cc(-c4ccccc4)c(C)cn3)c2)cn1]

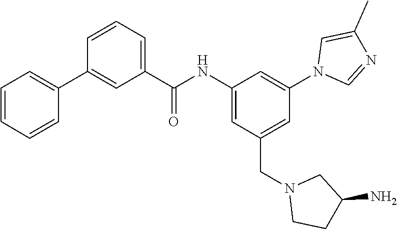 [SMILES: Cc1cn(-c2cc(CN3CC[C@H](N)C3)cc(NC(=O)c3cccc(-c4ccccc4)c3)c2)cn1]

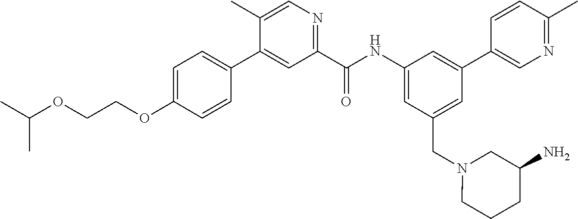 [SMILES: Cc1ccc(-c2cc(CN3CCC[C@H](N)C3)cc(NC(=O)c3cc(-c4ccc(OCCOC(C)C)cc4)c(C)cn3)c2)cn1]

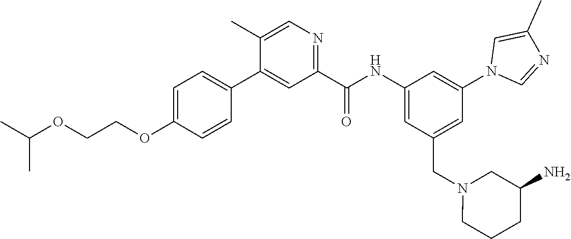 [SMILES: Cc1cn(-c2cc(CN3CCC[C@H](N)C3)cc(NC(=O)c3cc(-c4ccc(OCCOC(C)C)cc4)c(C)cn3)c2)cn1]

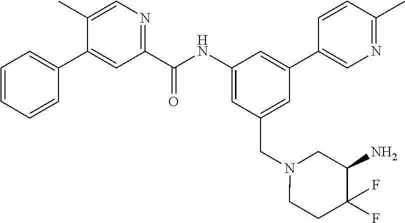 [SMILES: Cc1ccc(-c2cc(CN3CCC(F)(F)[C@H](N)C3)cc(NC(=O)c3cc(-c4ccccc4)c(C)cn3)c2)cn1]

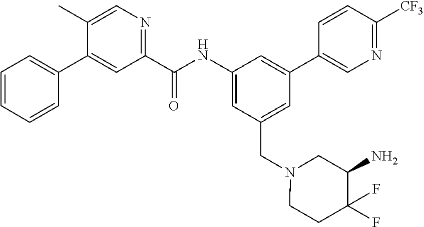 [SMILES: Cc1cnc(C(=O)Nc2cc(CN3CCC(F)(F)[C@H](N)C3)cc(-c3ccc(C(F)(F)F)nc3)c2)cc1-c1ccccc1]

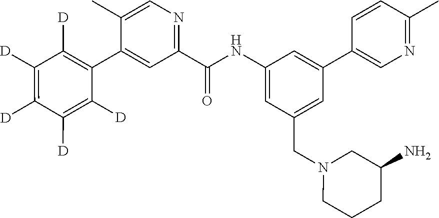 [SMILES: [2H]c1c([2H])c([2H])c(-c2cc(C(=O)Nc3cc(CN4CCC[C@H](N)C4)cc(-c4ccc(C)nc4)c3)ncc2C)c([2H])c1[2H]]

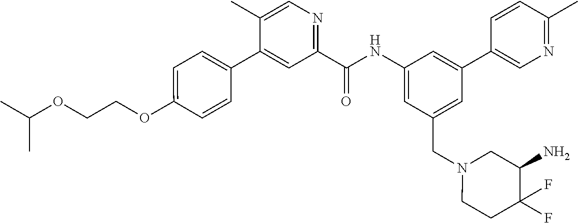 [SMILES: Cc1ccc(-c2cc(CN3CCC(F)(F)[C@H](N)C3)cc(NC(=O)c3cc(-c4ccc(OCCOC(C)C)cc4)c(C)cn3)c2)cn1]

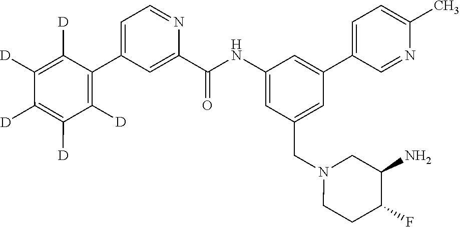 [SMILES: [2H]c1c([2H])c([2H])c(-c2ccnc(C(=O)Nc3cc(CN4CC[C@@H](F)[C@H](N)C4)cc(-c4ccc(C)nc4)c3)c2)c([2H])c1[2H]]

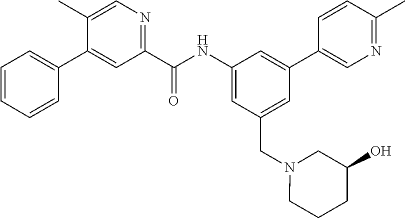 [SMILES: Cc1ccc(-c2cc(CN3CCC[C@H](O)C3)cc(NC(=O)c3cc(-c4ccccc4)c(C)cn3)c2)cn1]

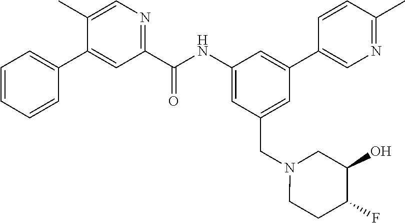 [SMILES: Cc1ccc(-c2cc(CN3CC[C@@H](F)[C@H](O)C3)cc(NC(=O)c3cc(-c4ccccc4)c(C)cn3)c2)cn1]

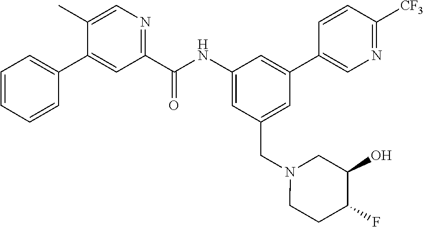 [SMILES: Cc1cnc(C(=O)Nc2cc(CN3CC[C@@H](F)[C@H](O)C3)cc(-c3ccc(C(F)(F)F)nc3)c2)cc1-c1ccccc1]

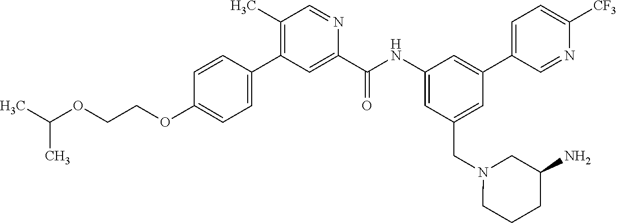 [SMILES: Cc1cnc(C(=O)Nc2cc(CN3CCC[C@H](N)C3)cc(-c3ccc(C(F)(F)F)nc3)c2)cc1-c1ccc(OCCOC(C)C)cc1]

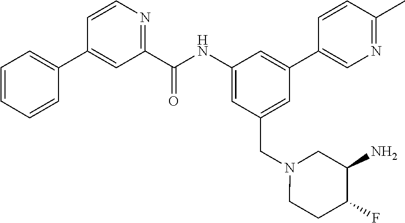 [SMILES: Cc1ccc(-c2cc(CN3CC[C@@H](F)[C@H](N)C3)cc(NC(=O)c3cc(-c4ccccc4)ccn3)c2)cn1]

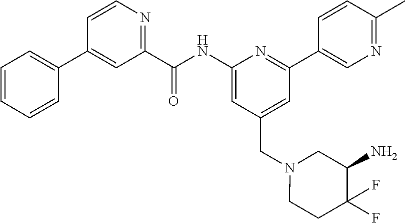 [SMILES: Cc1ccc(-c2cc(CN3CCC(F)(F)[C@H](N)C3)cc(NC(=O)c3cc(-c4ccccc4)ccn3)n2)cn1]

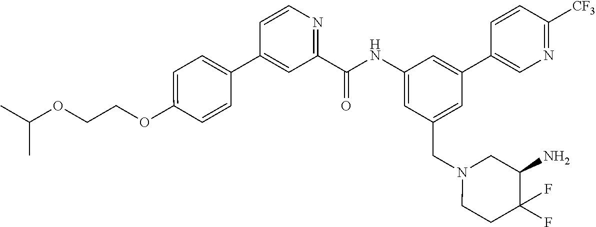 [SMILES: CC(C)OCCOc1ccc(-c2ccnc(C(=O)Nc3cc(CN4CCC(F)(F)[C@H](N)C4)cc(-c4ccc(C(F)(F)F)nc4)c3)c2)cc1]

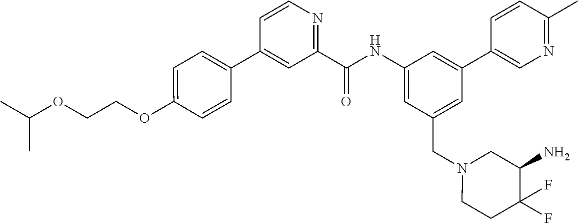 [SMILES: Cc1ccc(-c2cc(CN3CCC(F)(F)[C@H](N)C3)cc(NC(=O)c3cc(-c4ccc(OCCOC(C)C)cc4)ccn3)c2)cn1]

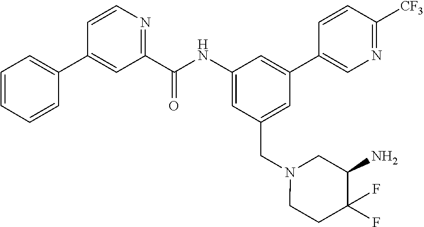 [SMILES: N[C@@H]1CN(Cc2cc(NC(=O)c3cc(-c4ccccc4)ccn3)cc(-c3ccc(C(F)(F)F)nc3)c2)CCC1(F)F]

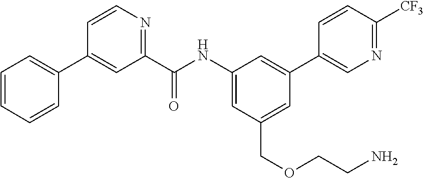 [SMILES: NCCOCc1cc(NC(=O)c2cc(-c3ccccc3)ccn2)cc(-c2ccc(C(F)(F)F)nc2)c1]

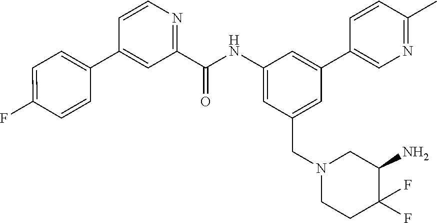 [SMILES: Cc1ccc(-c2cc(CN3CCC(F)(F)[C@H](N)C3)cc(NC(=O)c3cc(-c4ccc(F)cc4)ccn3)c2)cn1]

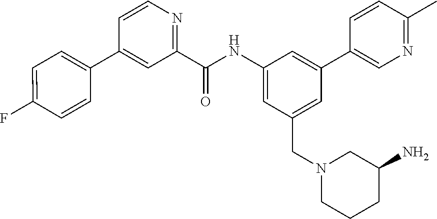 [SMILES: Cc1ccc(-c2cc(CN3CCC[C@H](N)C3)cc(NC(=O)c3cc(-c4ccc(F)cc4)ccn3)c2)cn1]

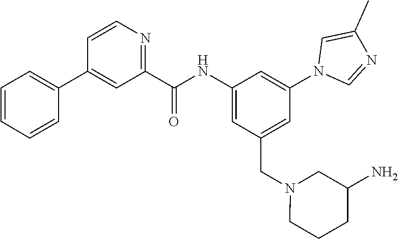 [SMILES: Cc1cn(-c2cc(CN3CCCC(N)C3)cc(NC(=O)c3cc(-c4ccccc4)ccn3)c2)cn1]

In a particularly preferred embodiment, the compound, pharmaceutically acceptable salt, solvate, prodrug or polymorph thereof is selected from

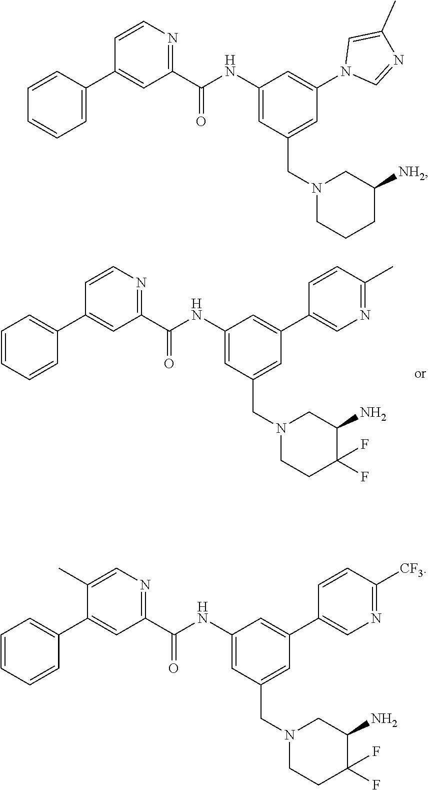

In a particularly preferred embodiment, the compound salt, solvate, prodrug or polymorph thereof is selected from

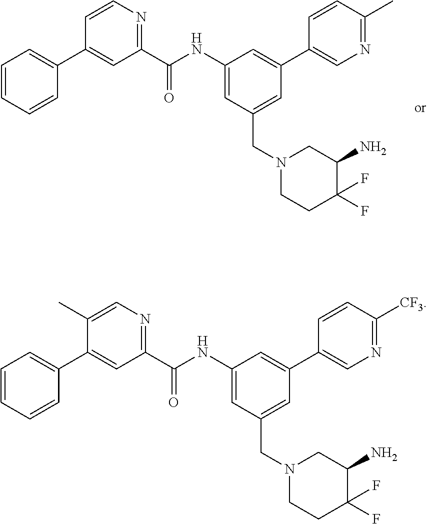 [SMILES: Cc1ccc(-c2cc(CN3CCC(F)(F)[C@H](N)C3)cc(NC(=O)c3cc(-c4ccccc4)ccn3)c2)cn1 ; Cc1cnc(C(=O)Nc2cc(CN3CCC(F)(F)[C@H](N)C3)cc(-c3ccc(C(F)(F)F)nc3)c2)cc1-c1ccccc1]

In some embodiments, the compounds may not inhibit kinase activity at physiologically relevant concentrations, particularly c-KIT, SRC, ABL and PDGFR kinases.

### Definitions of Nomenclature

As used herein the term “alkyl” refers to a straight or branched chain hydrocarbon radical having from one to twelve carbon atoms, or any range between, i.e. it contains 1, 2, 3, 4, 5, 6, 7, 8, 9, 10, 11 or 12 carbon atoms. The alkyl group is optionally substituted with substituents, multiple degrees of substitution being allowed.

Examples of “alkyl” as used herein include, but are not limited to, methyl, ethyl, n-propyl, isopropyl, n-butyl, isobutyl, t-butyl, n-pentyl, isopentyl, and the like.

As used herein, the terms “C₁-C₂ alkyl”, “C₁-C₄ alkyl”, “C₂-C₆ alkyl” and “C₁-C₆ alkyl” refer to an alkyl group, as defined above, containing at least 1, and at most 2, 4 or 6 carbon atoms respectively, or any range in between (e.g. alkyl groups containing 2-5 carbon atoms are also within the range of C₁-C₆).

As used herein the term “alkenyl” refers to an alkyl group containing a double bond. It may also be optionally substituted with substituents, multiple degrees of substitution being allowed.

As used herein, the term “halogen” refers to fluorine (F), chlorine (Cl), bromine (Br), or iodine (I) and the term “halo” refers to the halogen radicals fluoro (—F), chloro (—Cl), bromo (—Br), and iodo (—I). Preferably, ‘halo’ is fluoro or chloro.

As used herein, the term “haloalkyl” refers to an alkyl group as defined herein substituted with at least one halogen.

As used herein, the terms “C₁-C₂ haloalkyl”, “C₁-C₄ haloalkyl” and “C₁-C₆ haloalkyl” refer to an haloalkyl group, as defined herein, containing at least 1, and at most 2, 4 or 6 carbon atoms respectively, or any range in between (e.g. haloalkyl groups containing 2-5 carbon atoms are also within the range of C₁-C₆).

For example a C₁ haloalkyl group could be, but is not limited to, chloromethyl, or dichloromethyl, or trichloromethyl.

As used herein the term haloalkoxy refers to an alkoxy group as defined herein substituted with at least one halogen.

As used herein the term “fluoroalkyl” refers to a straight or branched chain hydrocarbon radical having from one to twelve carbon atoms or any range between i.e. it contains 1, 2, 3, 4, 5, 6, 7, 8, 9, 10, 11 or 12 carbon atoms and wherein at least one of the hydrogen atoms is substituted by a fluorine, All of the hydrogen atoms may be substituted by a fluorine. The fluoroalkyl group is optionally substituted with substituents. Examples of “fluoroalkyl” as used herein include, but are not limited to, fluoromethyl, difluoromethyl, trifluoromethyl, fluroethyl, difluoroethyl, trifluoroethyl, perfluoroethyl and the like.

As used herein the terms “C₁-C₂ fluoro alkyl”, “C₁-C₄ fluoralkyl” and “C₁-C₆ fluoroalkyl” refer to an fluoroalkyl group, as defined above, containing at least 1, and at most 2, 4 or 6 carbon atoms respectively, or any range in between (e.g. fluoroalkyl groups containing 2-5 carbon atoms are also within the range of C₁-C₆).

As used herein, the term “alkoxy” refers to an alkyl group as defined herein covalently bound via an O linkage. The alkoxy group is optionally substituted with substituents. Examples of “alkoxy” as used herein include, but are not limited to methoxy, ethoxy, propoxy, isoproxy, butoxy, iso-butoxy, tert-butoxy and pentoxy.

As used herein, the terms “C₁-C₂ alkoxy”, “C₁-C₄ alkoxy” and “C₁-C₆ alkoxy” refer to an alkoxy group, as defined herein, containing at least 1, and at most 2, 4 or 6 carbon atoms respectively, or any range in between (eg alkoxy groups containing 2-5 carbon atoms are also within the range of C₁-C₆).

As used herein, the term “cycloalkyl” refers to a non-aromatic cyclic hydrocarbon ring. In a like manner the term “C₃-C₇ cycloalkyl” refers to a non-aromatic cyclic hydrocarbon ring having from three to seven carbon atoms, or any range in between. For example, the C₃-C₇ cycloalkyl group would also include cycloalkyl groups containing 6 to 7 carbon atoms. The alkyl group is as defined above, and may be substituted. The cycloalkyl group refers to a nonaromatic cyclic ring, being saturated or having one or more degrees of unsaturation. Exemplary “C₃-C₇ cycloalkyl” groups useful in the present invention include, but are not limited to, cyclopropyl, cyclobutyl, cyclopentyl, cyclohexyl and cycloheptyl.

As used herein, the terms “heterocyclic” or “heterocyclyl” refer to a nonaromatic heterocyclic ring, being saturated or having one or more degrees of unsaturation, containing one or more heteroatom substitutions selected from S, S(O), S(O)₂, O, N, Si(RₐR_{b}), P, P(O)RₐR_{b}, or B(OR_{c}), wherein Rₐ and R_{b} are C₁-C₆ alkyl or aryl, or together with the atom between them form a 5- or 6-membered heterocyclyl ring, and R_{c} is hydrogen or C₁-C₆ alkyl. The term “C₃-C₇ heterocyclyl” refers to a non-aromatic cyclic hydrocarbon ring having from three to seven carbon atoms containing one or more heteroatom substitutions as referred to herein. The heterocyclic moiety may be substituted, multiple degrees of substitution being allowed. The term “C₃-C₇ heterocyclyl” also includes heterocyclyl groups containing C₄-C₅, C₅-C₇, C₆-C₇, C₄-C₇, C₄-C₆ and C₅-C₆ carbon atoms. Preferably, the heterocyclic ring contains four to six carbon atoms and one or two heteroatoms. More preferably, the heterocyclic ring contains five carbon atoms and one heteroatom, or four carbon atoms and two heteroatom substitutions, or five carbon atoms and one heteroatom. Such a ring may be optionally fused to one or more other “heterocyclic” ring(s) or cycloalkyl ring(s). Examples of “heterocyclic” moieties include, but are not limited to, tetrahydrofuran, pyran, oxetane, 1,4-dioxane, 1,3-dioxane, piperidine, piperazine, N-methylpiperazinyl, 2,4-piperazinedione, pyrrolidine, imidazolidine, pyrazolidine, morpholine, thiomorpholine, tetrahydrothiopyran, tetrahydrothiophene, and the like.

As an example of substituted heterocyclic groups, the term “(C₁-C₃ alkyl)C₃-C₇ heterocyclyl” includes heterocyclyl groups containing an alkyl group of one to three carbons in length as a linker between the compound and the heterocycle, (e.g. —CH₂-heterocycle or —CH₂CH₂-heterocycle). These heterocycles may be further substituted.

Substituted cycloalkyl and heterocyclyl groups may be substituted with any suitable substituent as described below.

The term “amino” or “amine” refers to the group —NH₂.

The term “substituted amino” or “secondary amino” refers to an amino group having a hydrogen replaced with, for example a C₁-C₆ alkyl group (“C₁-C₆ alkylamino”), an aryl or aralkyl group (“arylamino”, “aralkylamino”) and so on. C₁-C₃ alkylamino groups are preferred, such as for example, methylamino (NHMe), ethylamino (NHEt) and propylamino (NHPr).

The term “C₁-C₆ alkylamino” refers to a amino group having a hydrogen replaced with a C₁-C₆ alkyl group, wherein the C₁-C₆ alkyl group is as herein defined.

The term “disubstituted amino” or “tertiary amino” refers to an amino group having the two hydrogens replaced with, for example a C₁-C₆alkyl group, which may be the same or different (“dialkylamino”), an aryl and alkyl group (“aryl(alkyl)amino”) and so on. Di(C₁-C₃alkyl)amino groups are preferred, such as for example, dimethylamino (NMe₂), diethylamino (NEt₂), dipropylamino (NPr₂) and variations thereof (eg N(Me)(Et) and so on).

The term “sulfonyl” refers to the group —SO₂H.

The term “substituted sulfonyl” refers to a sulfonyl group having the hydrogen replaced with, for example a C₁-C₆ alkyl group (“sulfonylC₁-C₆ alkyl”), an aryl (“arylsulfonyl”), an aralkyl (“aralkylsulfonyl”) and so on. Sulfonyl C₁-C₃ alkyl groups are preferred, such as for example, —SO₂Me, —SO₂Et and —SO₂Pr.

The term “sulfonylamido” or “sulfonamide” refers to the group —SO₂NH₂.

The term “substituted sulfonamido” or “substituted sulphonamide” refers to an sulfonylamido group having a hydrogen replaced with, for example a C₁-C₆ alkyl group (“sulfonylamidoC₁-C₆ alkyl”), an aryl (“arylsulfonamide”), aralkyl (“aralkylsulfonamide”) and so on. SulfonylamidoC₁-C₃ alkyl groups are preferred, such as for example, —SO₂NHMe, —SO₂NHEt and —SO₂NHPr and includes reverse sulfonamides thereof (e.g. —NHSO₂Me, —NHSO₂Et and —NHSO₂Pr).

The term “disubstituted sufonamido” or “disubstituted sulphonamide” refers to an sulfonylamido group having the two hydrogens replaced with, for example a C₁-C₆ alkyl group, which may be the same or different (“sulfonylamidodi(C₁-C₆ alkyl)”), an aralkyl and alkyl group (“sulfonamido(aralkyl)alkyl”) and so on. Sulfonylamidodi(C₁-C₃ alkyl) groups are preferred, such as for example, —SO₂NMe₂, —SO₂NEt₂ and —SO₂NPr₂ and variations thereof (eg —SO₂N(Me)Et and so on) and includes reserve sulfonamides thereof (eg —N(Me)SO₂Me and so on).

The term “carboxylate” or “carboxyl” refers to the group —COO— or —COOH.

The term “carbamate” or “carbomyl” refers to the group —OC(O)NH₂. The carbamate may be substituted, or may be disubstituted, for example with an alkyl group such as but not limited to C₁-C₆ alkyl.

The term “carbonate” refers to the group —OC(O)O— or —OC(O)OH.

The term “alkylcarbonate” as herein defined refers to a carbonate group having the hydrogen replaced with, for example a C₁-C₆ alkyl group, an aryl or aralkyl group (“arylcabonate” or “aralkylcabonater”) and so on. CO₃C₁-C₃alkyl groups are preferred, such as for example, methylcarbonate (CO₃Me), ethylcarbonate (CO₃Et) and propylcarbonate (CO₃Pr).

The term “ester” refers to a carboxyl group having the hydrogen replaced with, for example a C₁-C₆ alkyl group (“carboxylC₁-C₆ alkyl” or “alkylester”), an aryl or aralkyl group (“arylester” or “aralkylester”) and so on. CO₂C₁-C₃ alkyl groups are preferred, such as for example, methylester (CO₂Me), ethylester (CO₂Et) and propylester (CO₂Pr) and includes reverse esters thereof (eg —OC(O)Me, —OC(O)Et and —OC(O)Pr).

As used herein, the term “aryl” refers to an optionally substituted benzene ring or to an optionally substituted benzene ring system fused to one or more optionally substituted benzene rings to form, for example, anthracene, phenanthrene, or napthalene ring systems. Examples of “aryl” groups include, but are not limited to, phenyl, 2-naphthyl, 1-naphthyl, biphenyl, as well as substituted derivatives thereof. Preferred aryl groups include arylamino, aralkyl and aralkoxy groups.

As used herein, the term “heteroaryl” refers to a monocyclic five, six or seven membered aromatic ring, or to a fused bicyclic or tricyclic aromatic ring system comprising at least one monocyclic five, six or seven membered aromatic ring. These heteroaryl rings contain one or more nitrogen, sulfur, and/or oxygen heteroatoms, where N-oxides and sulfur oxides and dioxides are permissible heteroatom substitutions and may be optionally substituted with up to three members. Examples of “heteroaryl” groups used herein include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl, quinolinyl, isoquinolinyl, cinnolyl, phthalazyl, naphthyridinyl, benzofuranyl, benzothiophenyl, indolyl, indazolyl, benzimidazolyl, and substituted versions thereof. Preferred heteroaryl groups include isoquinolinyl, imidazolyl and oxazolyl groups.

A “substituent” as used herein, refers to a molecular moiety that is covalently bonded to an atom within a molecule of interest. For example, a “ring substituent” may be a moiety such as a halogen, alkyl group, or other substituent described herein that is covalently bonded to an atom, preferably a carbon or nitrogen atom, that is a ring member. The term “substituted,” as used herein, means that any one or more hydrogens on the designated atom is replaced with a selection from the indicated substituents, provided that the designated atom's normal valence is not exceeded, and that the substitution results in a stable compound, i.e., a compound that can be isolated, characterized and tested for biological activity.

The terms “optionally substituted” or “may be substituted” and the like, as used throughout the specification, denotes that the group may or may not be further substituted or fused (so as to form a polycyclic system), with one or more non-hydrogen substituent groups. Suitable chemically viable substituents for a particular functional group will be apparent to those skilled in the art.

Examples of substituents include but are not limited to C₁-C₆ alkyl, C₁-C₆ haloalkyl, C₁-C₆ haloalkoxy, C₁-C₆ hydroxyalkyl, C₃-C₇ heterocyclyl, C₃-C₇ cycloalkyl, C₁-C₆ alkoxy, C₁-C₆ alkylsulfanyl, C₁-C₆ alkylsulfenyl, C₁-C₆ alkylsulfonyl, C₁-C₆ alkylsulfonylamino, arylsulfonoamino, alkylcarboxy, alkylcarboxyamide, oxo, hydroxy, mercapto, amino, acyl, carboxy, carbamoyl, aryl, aryloxy, heteroaryl, aminosulfonyl, aroyl, aroylamino, heteroaroyl, acyloxy, aroyloxy, heteroaroyloxy, alkoxycarbonyl, nitro, cyano, halogen, ureido, C₁-C₆ perfluoroalkyl or phosphorus containing groups such as phosphine oxides, P(O)Rₐ, P(O)ORₐOR_{b}, P(O)RₐR_{b}, C₁-C₆ alkyl-P(O)RₐR_{b} or the like, wherein Rₐ and R_{b} are C₁-C₆ alkyl or aryl, or together with the atom between them form a 5- or 6-membered heterocyclyl ring.

Any of these groups may be further substituted by any of the above-mentioned groups, where appropriate. For example, alkylamino, or dialkylamino, C₁-C₆ alkoxy, etc.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. The term “stereoisomers” refers to compounds which have identical chemical constitution, but differ with regard to the arrangement of the atoms or groups in space. As used herein, the term “stereoisomer” includes but is not limited to diastereomers, enantiomers and atropisomers, as well as mixtures thereof such as racemic mixtures.

Where the compounds are chiral, the compound may exist as a racemic mixture, predominantly one enantiomer, or only one enantiomer.

The activity of the compounds of the invention was measured first in a binding assay wherein the compounds interfered with the above-mentioned protein-protein interaction between the LDLR and PCSK9. Selected compounds were then subjected to a functional, cell-based assay wherein positive activity was recorded as a measure of increase in the expression of the LDLR on the surface of human lymphocytes. This assay therefore demonstrated the link between the targeted molecular interaction and the intended consequence, namely, to increase LDLR expression on the surface of cells in order to capture LDL particles from plasma, thereby reducing LDL plasma levels

The compounds have demonstrated efficacy and the levels of LDLR have increased with their use. Accordingly, the present invention also provides for the use of these compounds in inhibiting PCSK9, preventing the protein-protein interaction between PCSK9 and LDLR, increasing LDLR expression on the surface of PCSK9-exposed human lymphocytes and in reducing LDL levels.

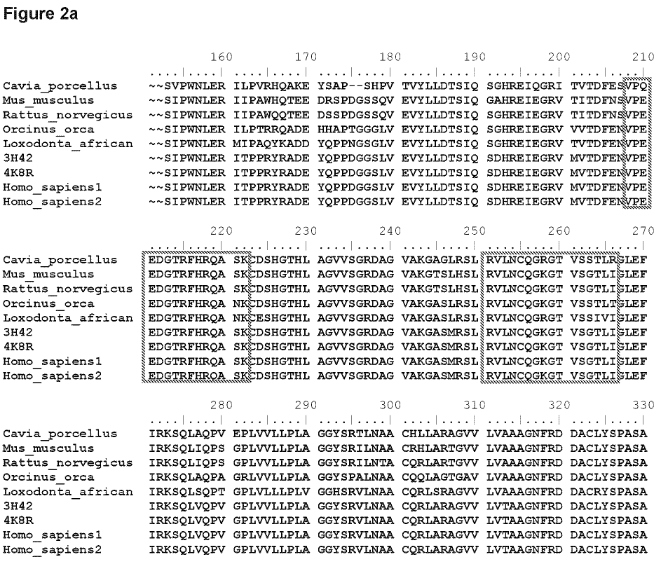

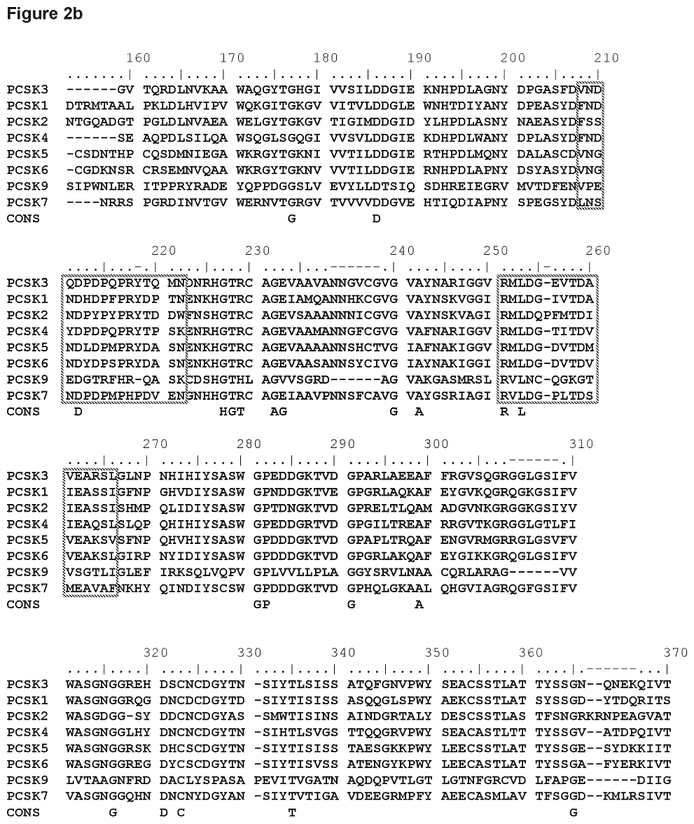

The targeted site is specific to the PCSK9 protein and the homology of this region is conserved across species. For example, it is conserved between humans, mice, rats, guinea pigs, pigs, elephants and killer whales (see FIG. 2a).

The PCSK family show very low levels of sequence identity. Cross-reactivity of the compounds with other PCSK molecules is therefore unlikely (See FIG. 2b).

### Compositions of the Invention

In one aspect, therefore, there is provided a composition comprising a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, and a pharmaceutically acceptable excipient.

As used herein, except where the context requires otherwise, the term “comprise” and variations of the term, such as “comprising”, “comprises” and “comprised”, are not intended to exclude further additives, components, integers or steps.

The term “pharmaceutically acceptable salt” as used herein refers to one or more salts of a given compound which possesses the desired pharmacological activity of the free compound and which are suitable for use in contact with the tissues of human and animals without undue toxicity, irritation or adverse response. Pharmaceutically acceptable salts are well known in the art. General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine.

In the case of compounds that are solids, it will be understood by those skilled in the art that the inventive compounds, agents and salts may exist in different crystalline or polymorphic forms, all of which are intended to be within the scope of the present invention and specified formulae.

The term “polymorph” includes any crystalline form of compounds of Formula (I) and/or Formula (II) such as anhydrous forms, hydrous forms, solvate forms and mixed solvate forms.

Formula (I), Formula (II), Formula (III), Formula (IIIa), Formula (IIIb), Formula (IVa), Formula (IVb), Formula (V) or combinations thereof are intended to cover, where applicable, solvated as well as unsolvated forms of the compounds. Thus, Formula (I), Formula (II), Formula (III), Formula (IIIa), Formula (IIIb), Formula (IVa), Formula (IVb), Formula (V) or Formula (VIa) or Formula (VIb) include compounds having the indicated structures, including the hydrated or solvated forms, as well as the non-hydrated and non-solvated forms.

As used herein, the term “solvate” refers to a complex of variable stoichiometry formed by a solute (in this invention, a compound of Formula (I), Formula (II), Formula (III), Formula (IIIa), Formula (IIIb), Formula (IVa), Formula (IVb), or Formula (V) Formula (VIa) or Formula (VIb) or a salt, prodrug or polymorph thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Basic nitrogen-containing groups may be quaternized with such agents as lower alkyl halide, such as methyl, ethyl, propyl, and butyl chlorides, bromides and iodides; dialkyl sulfates like dimethyl and diethyl sulfate; and others.

A “prodrug” is a compound that may not fully satisfy the structural requirements of the compounds provided herein, but is modified in vivo, following administration to a subject or patient, to produce a compound of formula (I) provided herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of compounds of Formula (I). The amino acid residues include the 20 naturally occurring amino acids commonly designated by three letter symbols and also include, 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) through the carbonyl carbon prodrug sidechain.

The compounds of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) and prodrugs thereof may be covalent irreversible or covalent reversible inhibitors of the active site of a protein.

Pharmaceutical compositions may be formulated from compounds according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) for any appropriate route of administration including, for example, topical (for example, transdermal or ocular), oral, buccal, nasal, vaginal, rectal or parenteral administration. The term parenteral as used herein includes subcutaneous, intradermal, intravascular (for example, intravenous), intramuscular, spinal, intracranial, intrathecal, intraocular, periocular, intraorbital, intrasynovial and intraperitoneal injection, as well as any similar injection or infusion technique. In certain embodiments, compositions in a form suitable for oral use or parenteral use are preferred. Suitable oral forms include, for example, tablets, troches, lozenges, aqueous or oily suspensions, dispersible powders or granules, emulsions, hard or soft capsules, or syrups or elixirs. For intravenous, intramuscular, subcutaneous, or intraperitoneal administration, one or more compounds may be combined with a sterile aqueous solution which is preferably isotonic with the blood of the recipient. Such formulations may be prepared by dissolving solid active ingredient in water containing physiologically compatible substances such as sodium chloride or glycine, and having a buffered pH compatible with physiological conditions to produce an aqueous solution, and rendering said solution sterile. The formulations may be present in unit or multi-dose containers such as sealed ampoules or vials. Examples of components are described in Martindale—The Extra Pharmacopoeia (Pharmaceutical Press, London 1993) and Martin (ed.), Remington's Pharmaceutical Sciences.

In a particularly preferred embodiment, the pharmaceutical compositions may be formulated from compounds according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) for oral administration, preferably at a dosage of 2-4.5 mg/kg, most preferably at a dosage of about 4.1 mg/kg.

Pharmaceutically acceptable carriers or diluents contemplated by the invention include any diluents, carriers, excipients, and stabilizers that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as plasma albumin, gelatine, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

### Uses of the Compounds and Compositions of the Invention

The compounds have demonstrated efficacy at inhibiting PCSK9 and the levels of LDL have been decreased with their use. Accordingly, the present invention also provides for the use of these compounds in inhibiting PCSK9, preventing the protein-protein interaction between PCSK9 and LDLR, and in reducing LDL levels.

The targeted site is specific to the PCSK9 protein and the homology of this region is conserved across species. For example, it is conserved between humans, mice, rats, guinea pigs, pigs, elephants and killer whales (see FIG. 2a). The PCSK family show very low levels of sequence identity. Cross-reactivity of the compounds with other PCSK molecules is therefore unlikely (See FIG. 2b).

In one aspect, therefore, there is provided a method for inhibiting PCSK9 in a subject in need thereof, the method comprising administering a therapeutically effective amount of a compound or a pharmaceutically acceptable salt, solvate, prodrug or polymorph thereof of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) to a subject.

By “therapeutically effective amount” it is meant the amount of a compound of the invention or a pharmaceutically acceptable salt, solvate, prodrug or polymorph thereof, that achieves the desired outcome in the subject. The desired outcome may be one or more of: complete or partial inhibition of PCSK9, reducing LDL, treating a disease or condition responsive to PCSK9 inhibition, and treating a disease or condition responsive to a reduction of LDL. The inhibition of PCSK9 and reduction of LDL is preferably to a level that results in a therapeutic effect including but not limited to delaying, slowing, stabilizing, curing, healing, alleviating, relieving, altering, remedying, less worsening, ameliorating, improving, or affecting the disease or condition or one or more symptoms thereof. Thus, in the context of the present disclosure the term “treating” encompasses any form of inhibition of PCSK9 that results in prevention, reduction or otherwise amelioration of the above-mentioned diseases or conditions, including complete and partial inhibition of PCSK9; and in the context of reducing the severity of elevated LDL levels, thereby resulting in the treatment or a reduced risk of cardiovascular diseases such as stroke, heart attack, coronary artery disease, hypercholesterolemia, and/or cerebrovascular diseases, atherosclerosis and/or associated diseases or their symptoms.

In the context of this specification the term “administering” and variations of that term including “administer” and “administration”, includes contacting, applying, delivering or providing a compound or composition of the invention to an organism, or a surface by any appropriate means.

The amount of active ingredient that is “effective” will vary from subject to subject, depending on the age and general condition of the individual, the particular active ingredient and the route of administration. Thus, it is not always possible to specify an exact “effective amount”. Preferred doses range from about 0.1 mg to about 140 mg per kilogram of body weight per day (e.g. about 0.5 mg to about 7 g per patient per day). The daily dose may be administered as a single dose or in a plurality of doses. The amount of active ingredient that may be combined with the carrier materials to produce a single dosage form will vary depending upon the subject treated and the particular mode of administration. Dosage unit forms will generally contain between about 1 mg to about 500 mg of an active ingredient. However, an appropriate “effective amount” in any individual case may be confirmed by one of ordinary skill in the art using routine experimentation.

In a preferred embodiment, the oral dose is 2 to 4.5 mg/kg.

“Subject” includes any human or non-human animal. Thus, in addition to being useful for human treatment, the compounds of the present invention may also be useful for veterinary treatment of mammals, including companion animals and farm animals, such as, but not limited to dogs, cats, horses, cows, sheep, and pigs.

As used herein, the term “subject” or “patient” can be used interchangeably with each other. The term “individual” or “patient” refers to an animal that is treatable by the compound and/or method, respectively, including but not limited to, for example, dogs, cats, horses, sheep, pigs, cows, and the like, as well as human, non-human primates. Unless otherwise specified, the “subject” or “patient” may include both male and female genders. Further, it also includes a subject or patient, preferably a human, suitable for receiving treatment with a pharmaceutical composition and/or method of the present invention.

In one aspect, there is provided a method for inhibiting PCSK9 in a subject in need thereof, the method comprising administering a therapeutically effective amount of a composition comprising a compound or a pharmaceutically acceptable salt, solvate, prodrug or polymorph thereof of Formula (I) or Formula (II) Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) to a subject.

In one aspect, there is provided a method for reducing LDL in a subject in need thereof, the method comprising administering a therapeutically effective amount of a compound or a pharmaceutically acceptable salt, solvate, prodrug or polymorph thereof of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) to a subject.

In one aspect, there is provided a method for reducing LDL in a subject in need thereof, the method comprising administering a therapeutically effective amount of a composition comprising a compound or a pharmaceutically acceptable salt, solvate, prodrug or polymorph thereof of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) to a subject.

Generally, the optimal level of LDL in a human adult is less than 100 mg/dL. LDL levels in the range of 100-129 mg/dL are considered as slightly elevated, 130-159 mg/dL are considered as borderline high, 160-189 mg/dL is considered as high and over 190 mg/dL as very high.

Accordingly, in one aspect of the invention, the patients receiving treatment have an LDL level greater than 100 mg/dL. In another embodiment, the patients receiving treatment will have an LDL level above 130 mg/dL. In another embodiment, the patients receiving treatment will have an LDL level above 160 mg/dL. In yet embodiment, the patients receiving treatment will have an LDL level above 190 mg/dL.

In one aspect, there is provided a method for treating a disease or condition in a subject in need thereof, wherein the disease or condition is any one of the following: cardiovascular disease, cerebrovascular disease, atherosclerosis and/or their associated diseases or their symptoms, the method comprising administering a therapeutically effective amount of a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof to a subject.

In one aspect, there is provided a method for treating a disease or condition in a subject in need thereof, wherein the disease or condition is any one of the following: cardiovascular disease, cerebrovascular disease, atherosclerosis and/or their associated diseases or their symptoms, the method comprising administering a therapeutically effective amount of a composition comprising a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof to a subject.

The terms ‘treatment’ or ‘treating’ of a subject includes the administration of a compound or composition, as described herein, to a subject for the purpose of delaying, slowing, stabilizing, curing, healing, alleviating, relieving, altering, remedying, less worsening, ameliorating, improving, or affecting the disease or condition or one or more symptoms thereof. The term “treating” further refers to any indication of success in the treatment including any objective or subjective parameter such as abatement; remission; lessening of the rate of worsening; lessening severity of the disease; stabilization, diminishing of symptoms or making the disease or condition more tolerable to the subject; slowing in the rate of degeneration or decline; making the final point of degeneration less debilitating; or improving a subject's physical or mental well-being.

As used herein, “preventing” or “prevention” is intended to refer to at least the reduction of likelihood of the risk of (or susceptibility to) acquiring a disease or disorder (i.e., causing at least one of the clinical signs or symptoms of the disease not to develop in an individual that may be exposed to or predisposed to the disease but does not yet experience or display signs or symptoms of the disease). Biological and physiological parameters for identifying such patients are provided herein and are also well known by physicians.

In another aspect, there is provided use of a compound of Formula (I) and/or Formula (II) or a pharmaceutically acceptable salt, solvate, prodrug or polymorph thereof, in the preparation of a medicament for the inhibition of PCSK9 in a subject.

In another aspect, there is provided use of a composition comprising a compound of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, in the preparation of a medicament for the inhibition of PCSK9 in a subject.

In another aspect, there is provided use of a compound of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, in the preparation of a medicament for the reduction of LDL in a subject.

In another aspect, there is provided use of a composition comprising a compound of Formula (I) or Formula (II)) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, in the preparation of a medicament for the reduction of LDL in a subject.

In another aspect, there is provided use of a compound of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof in the preparation of a medicament for the treatment of a disease or condition in a subject, wherein the disease or condition is any one of the following: cardiovascular disease, cerebrovascular disease, atherosclerosis and/or their associated diseases or their symptoms.

In another aspect, there is provided use of a composition comprising a compound of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof in the preparation of a medicament for the treatment of a disease or condition in a subject, wherein the disease or condition is any one of the following: cardiovascular disease, cerebrovascular disease, atherosclerosis and/or their associated diseases or their symptoms.

In another aspect, there is provided use of a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, for the inhibition of PCSK9.

In another aspect, there is provided use of a composition comprising a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, for inhibiting PCSK9.

In another aspect, there is provided use of a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, for the reduction of LDL.

In another aspect, there is provided use of a composition comprising a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, for the reduction of LDL.

In another aspect, there is provided use of a compound Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, for the treatment of a disease or condition in a subject, wherein the disease or condition is any one of the following: cardiovascular disease, cerebrovascular disease, atherosclerosis and/or their associated diseases or their symptoms.

In another aspect, there is provided use of a composition comprising a compound Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, for the treatment of a disease or condition in a subject, wherein the disease or condition is any one of the following: cardiovascular disease, cerebrovascular disease, atherosclerosis and/or their associated diseases or their symptoms.

In yet another aspect, there is provided a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, for use in inhibiting PCSK9.

In another aspect, there is provided a composition comprising a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, for use in inhibiting PCSK9.

In another aspect, there is provided a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, for use in reducing LDL.

In another aspect, there is provided a composition comprising a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, for use in reducing LDL.

In another aspect, there is provided a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, for use in the treatment of a disease or condition in a subject, wherein the disease or condition is any one of the following: cardiovascular disease, cerebrovascular disease, atherosclerosis and/or their associated diseases or their symptoms.

In another aspect, there is provided a composition comprising a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, for use in the treatment of a disease or condition in a subject, wherein the disease or condition is any one of the following: cardiovascular disease, cerebrovascular disease, atherosclerosis and/or their associated diseases or their symptoms.

In yet another aspect, there is provided a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, when used for inhibiting PCSK9.

In yet another aspect, there is provided a composition comprising a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, when used for inhibiting PCSK9.

In yet another aspect, there is provided a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, when used for reducing LDL.

In yet another aspect, there is provided a composition comprising a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, when used for reducing LDL.

In yet another aspect, there is provided a compound of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, when used for the treatment of a disease or condition in a subject, wherein the disease or condition is any one of the following: cardiovascular disease, cerebrovascular disease, atherosclerosis and/or their associated diseases or their symptoms.

In yet another aspect, there is provided a composition comprising a compound of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof, when used for the treatment of a disease or condition in a subject, wherein the disease or condition is any one of the following: cardiovascular disease, cerebrovascular disease, atherosclerosis and/or their associated diseases or their symptoms.

As set out above, the term “pharmaceutically acceptable” may be used to describe any pharmaceutically acceptable salt, hydrate or prodrug, or any other compound which upon administration to a subject, is capable of providing (directly or indirectly) a compound of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or an active metabolite or residue thereof.

The compounds of the present invention may be administered along with a pharmaceutical carrier, diluent or excipient as described above.

The methods of the present disclosure can be used to prevent or treat elevated LDL levels, which may or not have been diagnosed as one of the diseases or conditions referred to above.

In another aspect, the compounds of the present invention may be used to treat patients with a high diastolic blood pressure. In one embodiment of the invention, the patient receiving the treatment may have a diastolic blood pressure greater than 80.

In another embodiment, the patient receiving the treatment may have a diastolic blood pressure greater than 90.

Diabetes can be associated with hypercholesterolemia, both in terms of a potential risk due to hypercholesterolemia or as a result of previous treatments, such as statin treatment. Accordingly, a high blood glucose level may represent a cohort of patients for which treatment using the compounds of the invention may be appropriate. For example, it may be beneficial to treat patients with high blood glucose levels who may or may not be considered to be diabetic with compounds of the present invention rather than with medication that can further increase the risk of diabetes and/or an even higher blood glucose level. Alternatively, such patients may benefit from a lower dose of the other treatment in combination with the compounds of the present invention, as discussed below.

Accordingly, in one aspect, the compounds of the present invention may be used to treat patients with a high blood glucose level. For the majority of healthy individuals, normal blood sugar levels are below 6.1 mmol/L (108 mg/dL) when fasting, and up to 7.8 mmol/L (140 mg/dL) two hours after eating. For patients with pre-diabetes, blood sugar levels are increased from between 6.1-6.9 mmol/L (108-125 mg/dL) or more when fasting, and between 7.8-11.0 mmol/L (140-199 mg/dL) or more two hours after eating. For patients with diabetes, blood sugar levels are increased to 7 mmol/L (126 mg/dL) or more when fasting, and 11.1 mmol/L (200 mg/dL) or more two hours after eating. In one aspect, therefore, the compounds of the present invention are particularly suited for patients with pre-diabetes or diabetes.

### Combination Therapy

As discussed above, the compounds of the present invention are useful in reducing LDL. The compounds provide this result by inhibiting PCSK9, which is a different mechanism of action to that of the statins. Consequently, these compounds may provide treatment for the diseases or conditions listed above for patients who do not want, or who are unable, to take statins on their own. This may be due, for example, to the side effects of the statins, or simply that the statins will be (or have been) ineffective at (sufficiently) treating the disease or condition, such as some forms of hypercholesterolemia.

Statins inhibit the synthesis of cholesterol being produced by the liver, thereby decreasing the amount of LDL. They increase activity of sterol regulatory element-binding protein 2 (SREBP-2), resulting in activation of both LDL receptor (LDLR) and PCSK9. Increased expression and secretion of PCSK9 binds LDLR, resulting in higher LDL-C. Thus, while statins reduce LDL, as HMGcoA inhibitors, their effect on SREBP-2 acts as a counterbalance.

The addition of PCKS9 inhibitors to statin therapies may therefore help override this mechanism. Accordingly, the compounds of the present invention may therefore also be used together with statins to provide a more effective reduction in LDL than the statins alone, or to enable a lower dose of the statins to be used to reach a similar efficacy. This could then result in more effective treatments and/or fewer side effects for the patient than treatment or prophylaxis with statins alone.

Accordingly, in one aspect, the invention also provides a composition comprising:
a compound of the present invention, or a pharmaceutically acceptable salt, solvate, prodrug or polymorph thereof; and
a statin.

In another aspect, the present invention provides a method for reducing LDL in a subject in need thereof, the method comprising administering a therapeutically effective amount of a composition comprising:
compound of formula (I) or formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof; and
a statin.

In one aspect, there is provided a method for treating a disease or condition in a subject in need thereof, wherein the disease or condition is any one of the following: cardiovascular disease, cerebrovascular disease, atherosclerosis and/or their associated diseases or their symptoms, the method comprising administering a therapeutically effective amount of a composition comprising:
a compound according to formula (I) or formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof; and
a statin.

In another aspect, the present invention provides use of a composition comprising:
compound of formula (I) or, formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof; and
a statin in the preparation of a medicament for reducing LDL in a subject.

In another aspect, there is provided use of a composition comprising:
a compound of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof; and
a statin
   in the preparation of a medicament for the treatment of a disease or condition in a subject, wherein the disease or condition is any one of the following: cardiovascular disease, cerebrovascular disease, atherosclerosis and/or their associated diseases or their symptoms.

In another aspect, the present invention provides use of a composition comprising:
compound of formula (I) or formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb) or a pharmaceutically acceptable salt, solvate, prodrug or polymorph thereof; and
a statin for reducing LDL.

In another aspect, there is provided use of a composition comprising:
a compound Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb), or a pharmaceutically acceptable salt, solvate, prodrug or polymorph thereof, and
a statin
   for the treatment of a disease or condition in a subject, wherein the disease or condition is any one of the following: cardiovascular disease, cerebrovascular disease, atherosclerosis and/or their associated diseases or their symptoms.

In another aspect, the present invention provides use of a composition comprising:
compound of formula (I) or formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb), or a pharmaceutically acceptable salt, solvate, prodrug or polymorph thereof; and
a statin
   for use in reducing LDL.

In another aspect, there is provided a composition comprising:
a compound according to Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb), or a pharmaceutically acceptable salt, solvate, prodrug or polymorph thereof, and
a statin
   for use in the treatment of a disease or condition in a subject, wherein the disease or condition is any one of the following: cardiovascular disease, cerebrovascular disease, atherosclerosis and/or their associated diseases or their symptoms.

In another aspect, the present invention provides use of a composition comprising:
compound of formula (I) or formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb), or a pharmaceutically acceptable salt, solvate, prodrug or polymorph thereof; and
a statin
   when used for reducing LDL.

In yet another aspect, there is provided a composition comprising:
a compound of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb), or a pharmaceutically acceptable salt, solvate, prodrug or polymorph thereof, and
a statin
   when used for the treatment of a disease or condition in a subject, wherein the disease or condition is any one of the following: cardiovascular disease, cerebrovascular disease, atherosclerosis and/or their associated diseases or their symptoms.

The statins referred to in these aspects of the invention can include any statin that is approved for medical use. For example, the following statins may be used: atorvastatin (Lipitor), fluvastatin (Lescol, Lescol XL), lovastatin (Mevacor, Altoprev), pravastatin (Pravachol), rosuvastatin (Crestor), simvastatin (Zocor), and pitavastatin (Livalo).

Alternatively, a compound of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb), or a pharmaceutically acceptable salt, solvate, prodrug or polymorph thereof can be used in a combination with fibrates, bempedoic acid, resins or ezetimibe (Zetia):
Like the statins, fibrates reduce the body's cholesterol production. For example, a compound of Formula (I) and/or Formula (II), or a pharmaceutically acceptable salt, solvate, prodrug or polymorph thereof can be used in a combination with gemfibrozil (Lopid), clofibrate (Atromid-S) or fenofibrate (TriCor).
Bempedoic acid, like statins, targets the cholesterol biosynthesis pathway in the liver. Whereas statins inhibit HMG CoA reductase, bempedoic acid inhibits ATP-citrate lyase (ACL), two steps upstream of HMG CoA reductase.
Unlike other cholesterol-lowering medications, which are absorbed into the body, resins (which are also referred to as bile acid sequestrants and may be used interchangeably) remain in the intestinal tract, where they latch onto bile acids, preventing them from being absorbed into the bloodstream. Because the liver uses bile acids to produce cholesterol, the net effect is to lower LDL levels. A compound of Formula (I) and/or Formula (II), or a pharmaceutically acceptable salt, solvate, prodrug or polymorph thereof can be used in a combination with a resin such as colesevelam (WelChol), cholestyramine (Locholest) or colestipol (Colestid).
A type of cholesterol-lowering drug, ezetimibe (Zetia), acts by blocking the intestinal absorption of cholesterol in a different way than the resins do. On its own, ezetimibe can reduce LDL cholesterol levels; when added to a statin drug, ezetimibe can produce additional LDL reductions. A compound of Formula (I) and/or Formula (II), or a pharmaceutically acceptable salt, solvate, prodrug or polymorph thereof can be used in a combination with ezetimibe, or ezetimibe and a statin.

Compositions that are a combination of a compound of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb), or a pharmaceutically acceptable salt, solvate, prodrug or polymorph thereof with fibrates, bempedoic acid, resins and ezetimibe (optionally with a statin) can be used in methods and medicaments to reduce LDL in a subject and to treat a disease or condition in a subject, wherein the disease or condition is any one of the following: cardiovascular disease, cerebrovascular disease, atherosclerosis and/or their associated diseases or their symptoms.

As would be understood by the skilled person, the combination therapies may involve administering compounds via different administration routes. For example, a compound of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb), or a pharmaceutically acceptable salt, solvate, prodrug or polymorph thereof may be formulated for oral administration and the other active in the combination therapy may be administered intravenously. The two or more components of the combination therapy may be administered simultaneously or sequentially.

Accordingly, the combination therapies of the invention may be formulated as:
a single composition comprising a compound of Formula (I) or Formula (II) or Formula (III) or Formula (IIIa) or Formula (IIIb) or Formula (IVa) or Formula (IVb) or Formula (V) or Formula (Va) or Formula (Vb) or Formula (VIa) or Formula (VIb), or a pharmaceutically acceptable salt, solvate, prodrug or polymorph thereof with fibrates, bempedoic acid, resins and ezetimibe (optionally with a statin)
a composition for oral administration and a composition for non-oral administration. The two compositions may be administered simultaneously or sequentially.

It will be understood that the invention disclosed and defined in this specification extends to all alternative combinations of two or more of the individual features mentioned or evident from the text or drawings. All of these different combinations constitute various alternative aspects of the invention.

The methods and compounds described herein are described by the following illustrative and non-limiting examples. It will be understood that the invention disclosed and defined in this specification extends to all alternative combinations of two or more of the individual features mentioned or evident from the text or drawings. All of these different combinations constitute various alternative aspects of the invention.

### Definitions

TLC Thin layer chromatography
Prep-TLC Preparative thin layer chromatography
DIPEA Diisopropyl ethyl amine
TPP Triphenylphosphine
DIAD Diisopropyl azodicarboxylate
NBS N-bromosuccinimide
HATU O-(7-Azabenzotriazol-1-yl)-N,N,N′,N′-tetramethyluronium hexafluorophosphate
TFA Trifluoroacetic acid
DMF Dimethylformamide
mL milliliter(s)
mmol millimole(s)
h hour or hours
min minute or minutes
g gram(s)
mg milligram(s)
μL microlitres
eq equivalent(s
rt or RT room temperature, ambient, about 25° C.
MS mass spectrometry

### Experimental Procedure

Yields reported herein refer to purified products (unless specified) and are not optimized. All evaporations were carried out in vacuo with a rotary evaporator. Analytical samples were dried in vacuo (1-5 mmHg) at room temperature. Thin layer chromatography (TLC) was performed on silica gel plates, spots were visualized by UV light (214 and 254 nm). Purification by column and flash chromatography was carried out using silica gel (300-400 mesh). Solvent systems are reported as mixtures by volume. All NMR spectra were recorded on a Bruker 400 (400 MHz) spectrometer. ¹H chemical shifts are reported in δ values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration. LCMS spectra were obtained on an Agilent 1200 series 6110 or 6120 mass spectrometer with electrospray ionization and excepted as otherwise indicated, the general LCMS condition was as follows: Waters X Bridge C18 column (50 mm×4.6 mm×3.5 um), Flow Rate: 2.0 mL/min, the column temperature: 40° C.

### Example 1. Synthesis of compound 1-bromo-3-(bromomethyl)-5-nitrobenzene (29-B)

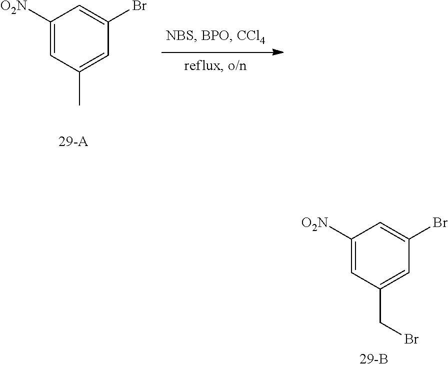

To a mixture of 29-A (25.0 g, 115.7 mmol) and NBS (21.6 g, 121.5 mmol) in CCl₄ (250 mL) was added benzoyl peroxide (2.80 g, 11.6 mmol), and the reaction mixture was heated to reflux and stirred overnight. After cooling to room temperature, the precipitate was removed by filtration and the filtrate was evaporated under reduced pressure to give a residue. The residue was purified by column chromatography on silica gel (eluting with 5% EA in PE) to give 29-B (27.0 g, 80% yield) as yellow solid.

### Example 2. Synthesis of Compound tert-butyl (S)-(1-3-bromo-5 nitrobenzyl)piperidin-3-yl)carbamate (29-C)

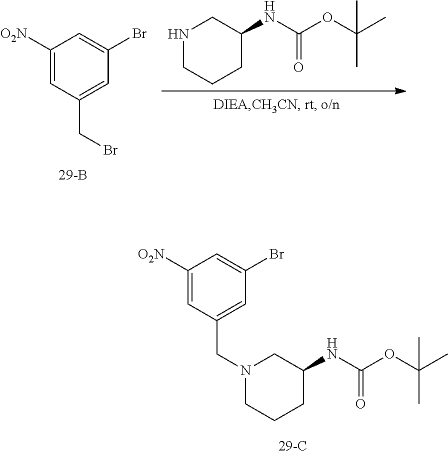

To a mixture of 29-B (27.0 g, 92.2 mmol), (S)-tert-butyl piperidin-3-ylcarbamate (18.4 g, 92.2 mmol) in CH₃CN (300 mL) was added DIEA (23.8 g, 184.4 mmol), and the reaction mixture was stirred at room temperature overnight. After consumption of the starting material, water (300 mL) was added and the mixture was extracted with EtOAc (2×300 mL). The combined organic layers were washed with brine (300 mL), dried over anhydrous Na₂SO₄ and concentrated under reduced pressure to give a residue. The residue was purified by column chromatography on silica gel (eluting with 15% EA in PE) to give 29-C (25.0 g, 66% yield) as yellow solid.

¹H NMR (400 MHz, CDCl₃) δ 8.19 (t, J=1.9 Hz, 1H), 8.04 (s, 1H), 7.74 (s, 1H), 4.82 (d, J=2.0 Hz, 1H), 3.71 (s, 1H), 3.51-3.38 (m, 2H), 2.54-2.52 (m, 1H), 2.30-2.24 (m, 3H), 1.63-1.50 (m, 3H), 1.37 (s, 9H).

### Example 3. The synthesis of compound tert-butyl (S)-(1-3-(4-methyl-1-imidazol-1-yl)-5-nitrobenzyl)piperidin-3-yl)carbamate (29-D)

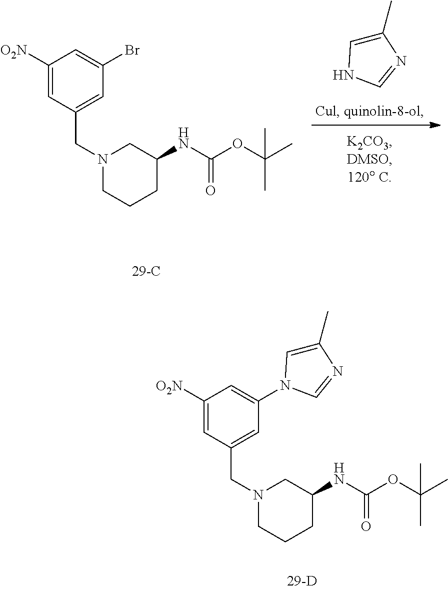

A suspension of 29-C (25.0 g, 60.5 mmol), 4-methyl-1H-imidazole (14.9 g, 181.5 mmol), K₂CO₃ (16.7 g, 121.0 mmol), CuI (3.5 g, 18.2 mmol) and quinolin-8-ol (1.8 g, 12.1 mmol) in DMSO (250 mL) was heated at 120° C. overnight under nitrogen atmosphere. After cooling to room temperature, water (300 mL) was added and the mixture was extracted with EtOAc (2×300 mL). The combined organic layers were washed with water (4×300 mL) and brine (300 mL) successively, dried over Na₂SO₄ and concentrated under reduced pressure to give a residue. The residue was purified by column chromatography on silica gel (eluting with 3% MeOH in DCM) to give 29-D (20.0 g, 99% yield) as yellow solid.

¹H NMR (400 MHz, DMSO-d₆) δ 8.35 (s, 1H), 8.33 (s, 1H), 8.08 (s, 1H), 8.02 (s, 1H), 7.66 (s, 1H), 6.78 (d, J=8.0 Hz, 1H), 3.70-3.60 (m, 2H), 3.44 (s, 1H), 2.77 (d, J=7.7 Hz, 1H), 2.62 (s, 1H), 2.18 (s, 3H), 2.00-1.97 (m, 1H), 1.88-1.85 (m, 1H), 1.73-1.60 (m, 2H), 1.54-1.40 (m, 1H), 1.35 (s, 9H), 1.23-1.10 (m, 1H).

### Example 4. The synthesis of tert-butyl (S)-(1-(3-amino-5-(4-methyl-1-imidazol-1-yl) benzyl)piperidin-3-yl)carbamate (29-E)

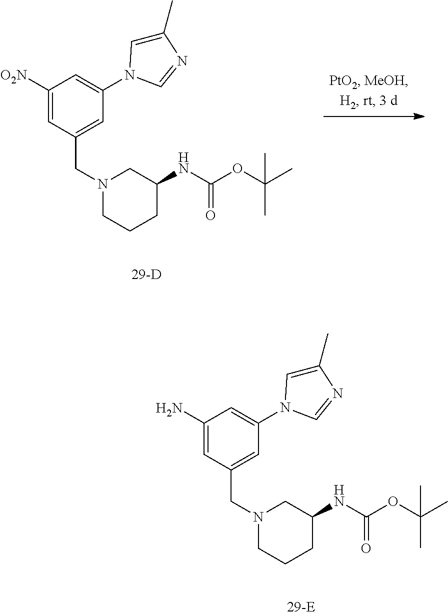 [SMILES: Cc1cn(-c2cc(CN3CCC[C@H](NC(=O)OC(C)(C)C)C3)cc([N+](=O)[O-])c2)cn1 ; Cc1cn(-c2cc(N)cc(CN3CCC[C@H](NC(=O)OC(C)(C)C)C3)c2)cn1]

To a solution of 29-D (54.1 g, 130.2 mmol) in MeOH (1.0 L) was added PtO₂ (2.0 g), and the reaction mixture was stirred at room temperature for 3 days under hydrogen atmosphere. After consumption of the starting material, the reaction mixture was filtered through celite to remove the solid and the filtrate was evaporated under reduced pressure to give crude 29-E (45.0 g, 90% yield) as black solid.

LC/MS (ESI): m/z=386.4 [M+1]⁺. RT=1.787 min

¹H NMR (400 MHz, DMSO-d₆) δ 6.71 (d, J=7.4 Hz, 1H), 6.63 (s, 1H), 6.49 (s, 1H), 5.35 (s, 2H), 3.37-3.34 (m, 2H), 3.17 (d, J=4.9 Hz, 1H), 2.76 (s, 1H), 2.63 (s, 1H), 2.54 (s, 3H), 1.85-1.83 (m, 1H), 1.72-1.68 (m, 2H), 1.62-1.59 (m, 1H), 1.35 (s, 9H), 1.28-1.26 (m, 1H), 1.18-1.06 (m, 1H).

### Example 5. The synthesis of tert-butyl (S)-(1-(3-(4-bromopicolinamido)-5-(4-methyl-1H-imidazol-1-yl)benzyl)piperidin-3-yl)carbamate (29-F)

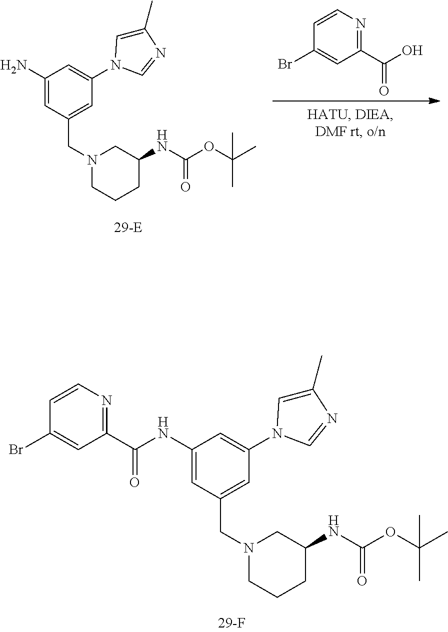 [SMILES: Cc1cn(-c2cc(N)cc(CN3CCC[C@H](NC(=O)OC(C)(C)C)C3)c2)cn1 ; O=C(O)c1cc(Br)ccn1 ; Cc1cn(-c2cc(CN3CCC[C@H](NC(=O)OC(C)(C)C)C3)cc(NC(=O)c3cc(Br)ccn3)c2)cn1]

To a solution of 29-E (45.0 g, 116.9 mmol) and 4-bromopicolinic acid (23.6 g, 116.9 mmol) in DMF (800 mL) was added HATU (53.3 g, 140.2 mmol) and DIEA (45.3 g, 350.6 mmol), and the reaction mixture was allowed to stir at room temperature overnight. After consumption of the starting material, the reaction mixture was diluted by water (800 mL) and extracted with EtOAc (2×800 mL). The organic extract was washed by water (4×1000 mL) and brine (1000 mL) successively, dried over anhydrous Na₂SO₄, filtered and evaporated under reduced pressure to give a residue. The residue was purified by column chromatography on silica gel (eluting with 3% MeOH in DCM) to give 29-F (45.0 g, 67% yield) as yellow solid.

LC/MS (ESI): m/z=568.8 [M+1]⁺. RT=2.241 min

### Example 6. The synthesis of compound tert-butyl (S)-(1-(3-(4-methyl-1H-imidazol-1-yl)-5-(4-phenylpicolinamido)benzyl)piperidin-3-yl)carbamate (29-G)

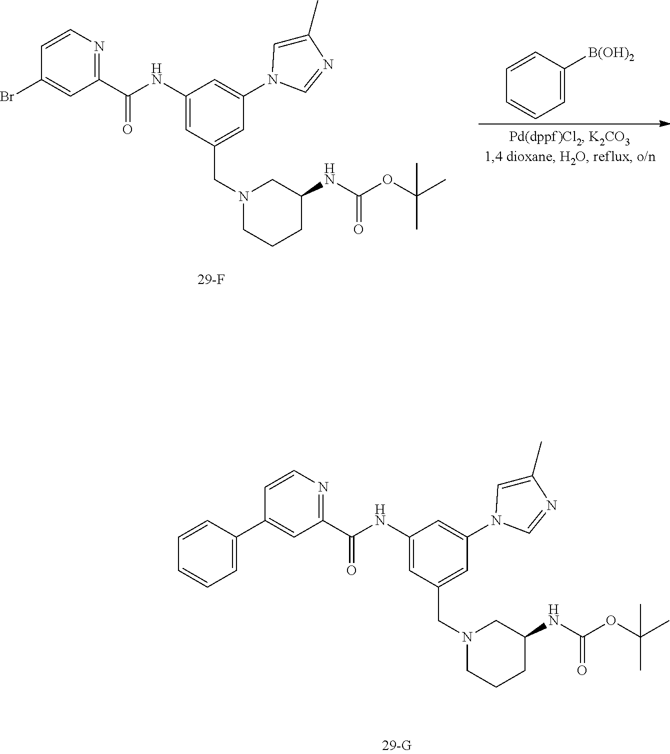 [SMILES: Cc1cn(-c2cc(CN3CCC[C@H](NC(=O)OC(C)(C)C)C3)cc(NC(=O)c3cc(Br)ccn3)c2)cn1 ; OB(O)c1ccccc1 ; Cc1cn(-c2cc(CN3CCC[C@H](NC(=O)OC(C)(C)C)C3)cc(NC(=O)c3cc(-c4ccccc4)ccn3)c2)cn1]

To a mixture of 29-F (40.0 g, 70.3 mmol), phenylboronic acid (12.9 g, 105.5 mmol), and K₂CO₃ (19.5 g, 140.6 mmol) in 1, 4-dioxane (500 mL) and H₂O (100 mL) was added Pd(dppf)Cl₂ (7.7 g, 10.5 mmol), and the reaction mixture was allowed to heat at reflux and stir overnight under argon atmosphere. After cooling to room temperature, it was diluted by water (500 mL) and extracted with EtOAc (2×400 mL). The organic extract was washed by brine (500 mL), dried over anhydrous Na₂SO₄ and evaporated under reduced pressure to give a residue. The residue was purified by column chromatography on silica gel (eluting with 3% MeOH in DCM) to give 29-G (20.0 g, 50% yield) as gray solid.

LC/MS (ESI): m/z=567.0 [M+1]⁺. RT=6.813 min

¹H NMR (400 MHz, DMSO-d₆) δ 10.84 (s, 1H), 8.82 (d, J=4.8 Hz, 1H), 8.43 (d, J=1.2 Hz, 1H), 8.08-8.03 (m, 3H), 7.96-7.90 (m, 3H), 7.61-7.54 (m, 3H), 7.41 (s, 1H), 7.28 (s, 1H), 6.74 (d, J=7.6 Hz, 1H), 3.58-3.43 (m, 3H), 2.79 (d, J=7.6 Hz, 1H), 2.69-2.66 (m, 1H), 2.18 (s, 3H), 1.97-1.91 (m, 1H), 1.85-1.81 (m, 1H), 1.72-1.65 (m, 1H), 1.51-1.45 (m, 1H), 1.35 (s, 9H), 1.27-1.13 (m, 2H).

### Example 7. The synthesis of compound (S)—N-(3-((3-aminopiperidin-1-yl)methyl)-5-(4-methyl-1H-imidazol-1-yl)phenyl)-4-phenylpicolinamide (compound 29)

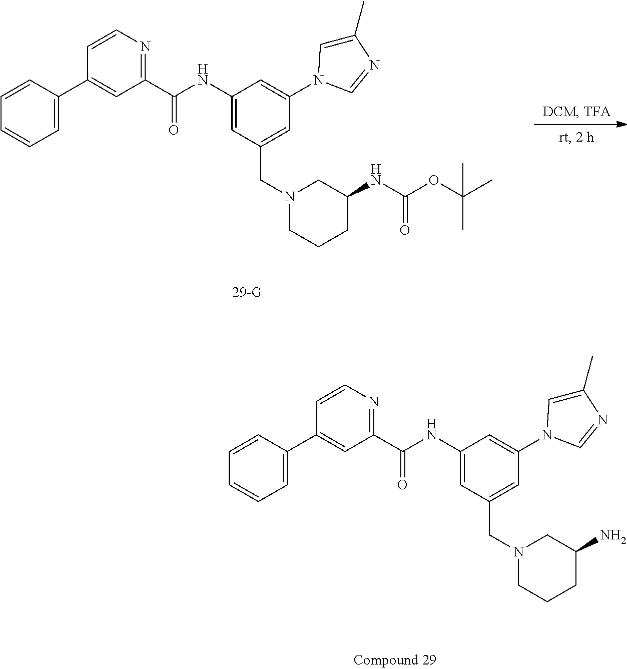 [SMILES: Cc1cn(-c2cc(CN3CCC[C@H](NC(=O)OC(C)(C)C)C3)cc(NC(=O)c3cc(-c4ccccc4)ccn3)c2)cn1 ; Cc1cn(-c2cc(CN3CCC[C@H](N)C3)cc(NC(=O)c3cc(-c4ccccc4)ccn3)c2)cn1]

To a solution of 29-G (20.0 g, 35.3 mmol) in DCM (150 mL) was added TFA (50 mL), and the reaction mixture was stirred at room temperature for 2 h. After consumption of the starting material, the volatiles were evaporated under reduced pressure to give a residue. The resulted residue was dissolved in DCM (300 mL) and basified by aqueous K₂CO₃ (300 mL). The organic layer was separated, washed by water (300 mL) and brine (300 mL) successively, dried over anhydrous Na₂SO₄, filtered and evaporated under reduced pressure to give compound 29 (10.5 g, 64% yield) as gray solid.

LC/MS (ESI): m/z=467.0 [M+1]⁺. RT=5.482 min

¹H NMR (400 MHz, DMSO-d₆) δ 10.84 (s, 1H), 8.82 (d, J=5.2 Hz, 1H), 8.43 (d, J=0.8 Hz, 1H), 8.08-8.00 (m, 3H), 7.97-7.90 (m, 3H), 7.61-7.52 (m, 3H), 7.40 (s, 1H), 7.27 (s, 1H), 3.54 (d, J=13.6 Hz, 1H), 3.46 (d, J=13.2 Hz, 1H), 2.75-2.67 (m, 3H), 2.18 (s, 3H), 1.97 (t, J=9.6 Hz, 1H), 1.74-1.61 (m, 3H), 1.51-1.43 (m, 1H), 1.01-0.94 (m, 1H).

### Example 8: Synthesis of Compounds of the Invention

Experimental Details:

General information: Yields reported herein refer to purified products (unless specified). All evaporations were carried out in vacuo with a rotary evaporator. Analytical samples were dried in vacuo (1-5 mm Hg) at room temperature. Thin layer chromatography (TLC) was performed on Merck silica gel 60 F₂₅₄ aluminium-backed silica gel plates, spots were visualized by UV light (214 and 254 nm) or iodine or ninhydrin or potassium permanganate solution followed by heating. Purification by column and flash chromatography was carried out using silica gel (300-400 mesh, unless specified otherwise). Solvent systems are reported as mixtures by volume. ¹H-NMR spectra were recorded on a Bruker 400 MHz spectrometer. ¹H chemical shifts are reported in δ values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration. LCMS spectra were obtained on an Agilent 1200 series 6110 or 6120 mass spectrometer with electrospray ionization and excepted as otherwise indicated, the general LCMS condition was as follows: Waters X Bridge C18 column (50 mm×4.6 mm×3.5 μm), Flow Rate: 2.0 mL/min, the column temperature: 40° C., unless specified otherwise.

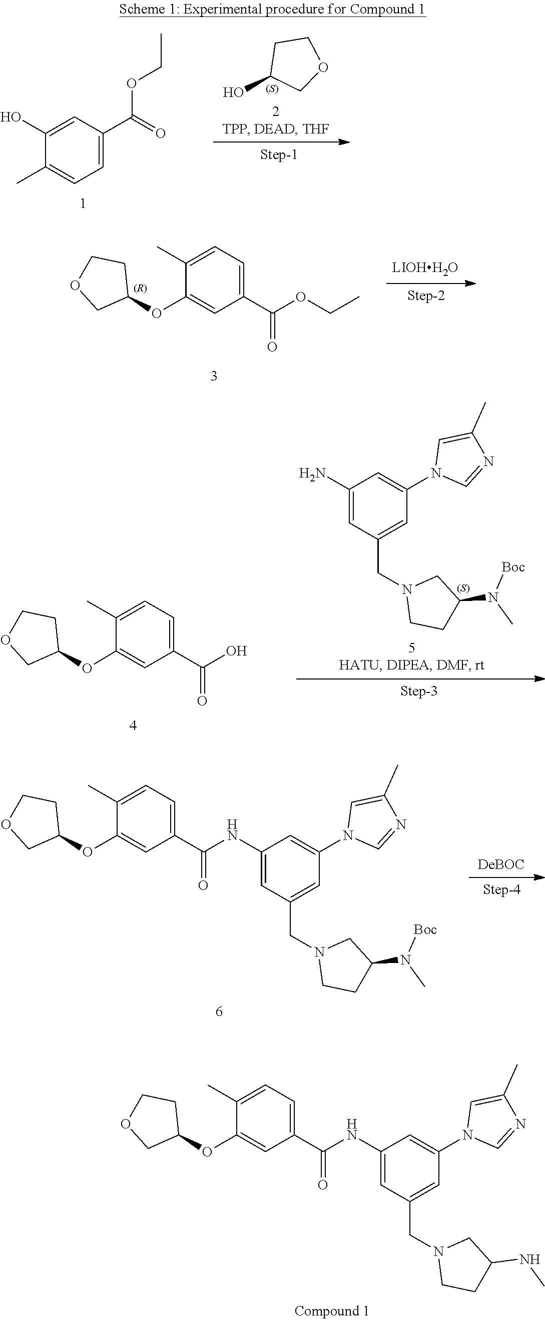

Step-1 Synthesis of ethyl (R)-4-methyl-3-((tetrahydrofuran-3-yl)oxy)benzoate (3): To a mixture of ethyl 3-hydroxy-4-methylbenzoate (1, 1 g, 6.02 mmol), (S)-tetrahydrofuran-3-ol (0.79 g, 9.05 mmol) and PPh₃ (2, 2.05 g, 7.83 mmol) in THF, DEAD (1.57 g, 9.03 mmol) was added at 0° C. The reaction mixture was stirred at 70° C. for 16 h. After consumption of starting material, the reaction mixture was concentrated in vacuo. The residue was purified by flash column chromatography [normal phase, silica gel (100-200 mesh), gradient 5% to 8% EtOAc in hexane] to give the title compound as a light-yellow solid (3, 850 mg, 59%). LC/MS: m/z 254.05 (M+18)+(ES+), 96.16%

Step-2 Synthesis of (R)-4-methyl-3-((tetrahydrofuran-3-yl)oxy)benzoic acid (4): To a solution of ethyl (R)-4-methyl-3-((tetrahydrofuran-3-yl)oxy)benzoate (3, 850 mg, 3.60 mmol) in THF:H₂O (12 mL: 3:1), LiOH·H₂O (4 g, 18 mmol) was added at 0° C. The reaction mixture was stirred at room temperature for 16 h. The reaction progress was monitored by TLC which showed consumption of starting material. The reaction mixture was concentrated in vacuo and the reaction mixture was acidified with 1N HCl (pH=2-3). The aqueous layer was extracted with ethyl acetate (2×60 mL). The organic layers were combined, dried (Na₂SO₄) and concentrated in vacuo to give the title compound (4, 600 mg, 75%) as a white solid. LCMS: m/z 220.9 (M−H)⁻ (ES−), 99.03%

Step-3 Synthesis of tert-butyl methyl((S)-1-(3-(4-methyl-1H-imidazol-1-yl)-5-(4-methyl-3-(((R)-tetrahydrofuran-3-yl)oxy)benzamido)benzyl)pyrrolidin-3-yl)carbamate (6): To a solution of amine 5 (205 mg, 0.93 mmol) and acid 4 (300 mg, 0.779 mmol) in DMF (10 mL), DIPEA (310 mg, 2.33 mmol) was added followed by addition of HATU (592 mg, 1.55 mmol) at 0° C. and the reaction mixture was stirred at room temperature for 16 h. Progress of the reaction was monitored by TLC, which showed consumption of starting material. After completion of reaction, the reaction mixture was diluted with water (50 mL) and extracted with EtOAc (2×50 ml), the resulting combined organic layer was dried (Na₂SO₄) and concentrated in vacuo to give the title compound (6, 510 mg crude) as brown solid. LCMS: m/z 590.45 (M+H)⁺ (ES+), 34%

Step-4 Synthesis of 4-methyl-N-(3-(4-methyl-1H-imidazol-1-yl)-5-(((S)-3-(methylamino)pyrrolidin-1-yl)methyl)phenyl)-3-(((R)-tetrahydrofuran-3-yl)oxy)benzamide (Compound 1): To a solution of 6 (510 mg crude) in 1,4-dioxane (5 mL), 4 M HCl in dioxane (5 mL) was added. After completion (monitored by TLC), the reaction mixture was concentrated in vacuo. The crude material was purified by prep-HPLC (reverse phase, X Select Phenyl Hexyl C18 (19×250) mm, 10 μm, gradient 40% to 60% ACN in water containing 5 mM Ammonium acetate, RT: 14.1 min to give the title compound (Compound 1, 48 mg, 35% over 2 steps) as an off-white solid.

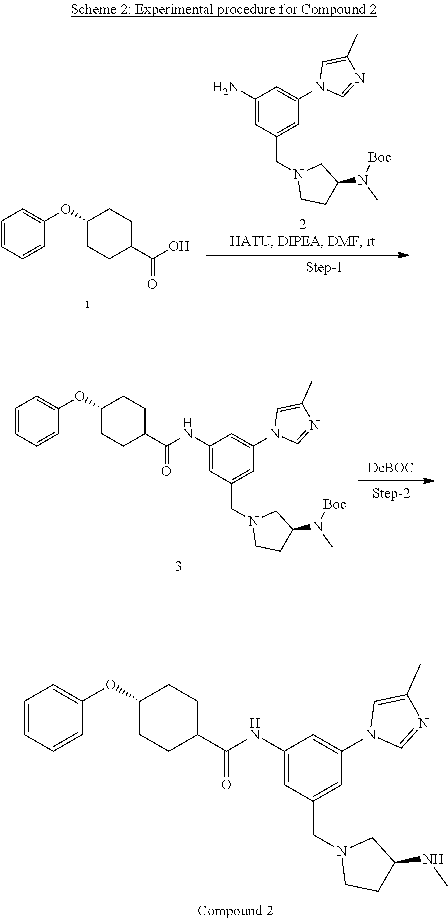

Step-1 Synthesis of tert-butyl methyl((3S)-1-(3-(4-methyl-1H-imidazol-1-yl)-5-(4-phenoxycyclohexane-1-carboxamido)benzyl)pyrrolidin-3-yl)carbamate (3): To a solution of amine 5 (110 mg, 0.28 mmol and acid 1 (70 mg, 0.314 mmol) in DMF (5 mL), DIPEA (110 mg, 0.85 mmol) was added followed by addition of HATU (216 mg, 0.57 mmol) at 0° C. The reaction mixture was stirred the at room temperature for 16 h. Progress of the reaction was monitored by TLC, which showed consumption of starting material. After completion of reaction, the reaction mixture was diluted with water (50 mL) and extracted with EtOAc (2×20 mL), the resulting organic layers was dried (Na₂SO₄) and concentrated in vacuo to give the title compound (3, 210 mg crude) as a brown solid. LCMS: m/z 588.41 (M+H)⁺ (ES+), 66%

Step-2 Synthesis of N-(3-(4-methyl-1H-imidazol-1-yl)-5-(((S)-3-(methylamino)pyrrolidin-1-yl)methyl)phenyl)-4-phenoxycyclohexane-1-carboxamide (Compound 2): To a solution of 3 (210 mg crude) in 1,4-dioxane (5 mL), 4 M HCl in dioxane (5 mL) was added. After completion (monitored by TLC), the reaction mixture was concentrated in vacuo. The crude material was purified by prep-HPLC (reverse phase) to give the title compound (Compound 2, 55 mg, 23% over 2 steps) as an off-white solid. LCMS: m/z 488.41 (M+H)⁺ (ES+), 99.66%.

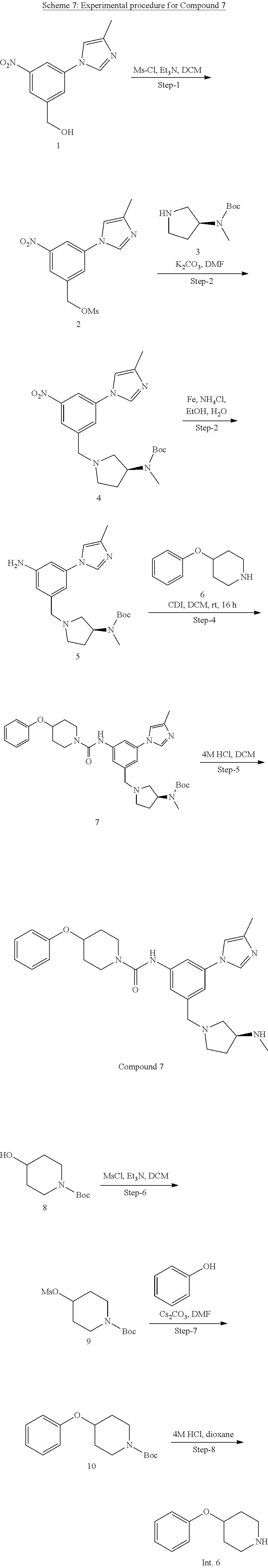

Step-1 Synthesis of 3-(4-methyl-1H-imidazol-1-yl)-5-nitrobenzyl methanesulfonate (2): To a solution of 1 (20 g, 85 mmol) in DCM (350 mL), Et₃N (57 mL, 34 mmol) was drop wise added at 0° C. After 15 min stirring at same temperature, methane sulfonylchloride (57 mL, 2.42 mmol) was slowly added at 0° C. and the reaction was continued at the room temperature for 1 h. After completion (monitored by TLC), the reaction mixture was quenched with water (200 mL) and compound was extracted with DCM (3×200 mL). The combined organic layer was washed with brine solution (50 mL), dried (Na₂SO₄), concentrated in vacuo to give the title compound (2, 20 g, 96%) as a sticky solid. LCMS: m/z 309.87 (M−H)⁻ (ES−), 99% ¹H NMR (400 MHz, CDCl₃): δ 2.17 (s, 3H), 4.92 (s, 2H), 7.64 (s, 1H), 8.21 (s, 1H), 8.25 (s, 1H), 8.35 (s, 1H), 8.41 (s, 1H).

Step-2 Synthesis of tert-butyl (S)-methyl(1-(3-(4-methyl-1H-imidazol-1-yl)-5-nitrobenzyl)pyrrolidin-3-yl)carbamate (4): To a solution of amine 3 (12.9 g, 64 mmol) in dry DMF (120 mL), K₂CO₃ (22 g, 16 mmol) was added. The reaction was stirred for 10 min and 2 (20 g, 60 mmol) was added at room temperature. The resulting mixture was stirred for 16 h at room temperature. After completion of reaction (monitored by TLC), water (200 mL) was added to the reaction mixture and the aqueous layer was extracted with ethyl acetate (3×100 mL). The organic layers were combined, dried (Na₂SO₄) and concentrated in vacuo. The residue was purified by flash column chromatography [normal phase, silica gel (100-200 mesh), gradient 30% to 40% EtOAc in Hexane] to give the title compound (4, 9.6 g, 40%) as a sticky solid. LCMS: m/z 360.16 (M−H)⁻ (ES−), 92.40%

Step-3 Synthesis of tert-butyl (S)-(1-(3-amino-5-(4-methyl-1H-imidazol-1-yl)benzyl)pyrrolidin-3-yl)(methyl)carbamate (5): A mixture of 4 (9.69 g, 23.0 mmol), NH₄Cl (7.41 g, 138 mmol) and Fe powder (11.53 g, 207 mmol) in ethanol (100 mL) were heated at 70° C. for 3 h. After complete consumption of the starting material (monitored by TLC), the reaction mixture was filtered through a pad of celite, washed with 10% MeOH in DCM (300 mL) and concentrated in vacuo. The residue obtained was dissolved in 20% IPA/CHCl₃ (200 mL), washed with H₂O (50 mL). The combined organic layers were dried (Na₂SO₄) and concentrated in vacuo to give the title compound (5, 8.1 g, 92%) as sticky solid. LCMS: m/z 386.26 (M+H)⁺ (ES+), 89.5%

Step-4 Synthesis of tert-butyl (S)-methyl(1-(3-(4-methyl-1H-imidazol-1-yl)-5-(4-phenoxypiperidine-1-carboxamido)benzyl)pyrrolidin-3-yl)carbamate (7): To a solution of 5 (150 mg, 0.389 mmol) in DCM, CDI (94 mg, 0.584 mmol) was added to it and the reaction mixture was stirred at room temperature for 1 h. Then, 4-phenoxypiperidine (6, 103 mg, 0.54 mmol) was added and the resulting reaction mixture was stirred at room temperature for 16 h. After completion of reaction (monitored by TLC), the reaction was quenched with water (20 mL) and extracted with DCM (2×20 mL). The combined organic layer was dried (Na₂SO₄) and concentrated in vacuo to give the title compound (7, 200 mg crude) as an oily mass which was used to the next step without purification. LCMS: m/z 587.46 (M−H)⁻ (ES−), 23%

Step-5 Synthesis of (S)—N-(3-(4-methyl-1H-imidazol-1-yl)-5-((3-(methylamino)pyrrolidin-1-yl)methyl)phenyl)-4-phenoxypiperidine-1-carboxamide (Compound 7): To a solution of 7 (200 mg crude) in 1,4-dioxane (5 mL), 4 M HCl in dioxane was added. After consumption of starting material (monitored by TLC), the reaction mixture was concentrated in vacuo. The crude material was purified by prep-HPLC (reverse phase, X Terra C18 (19×250) mm, 10 μm, gradient 62% to 38% ACN in water containing 5 mM Ammonium acetate, RT: 9.82 min to give the title compound (Compound 7, 15 mg, 8% over 2 steps) as an off-white solid.

Step-6 Synthesis of tert-butyl 4-((methylsulfonyl)oxy)piperidine-1-carboxylate (9): To a solution of 8 (3 g, 14.9 mmol) in DCM (60 mL), Et₃N (2.2 g, 17.9 mmol) was drop wise added at 0° C. After 15 min stirring at same temperature, methane sulfonylchloride (2.2 g, 22.3 mmol) was slowly added at 0° C. and the reaction was continued at the room temperature for 1 h. After completion (monitored by TLC), the reaction mixture was quenched with water (50 mL) and compound was extracted with DCM (3×100 mL). All the organic layer was washed with brine solution (50 mL), dried (Na₂SO₄), concentrated in vacuo to give the title compound (9, 2.8 g, crude) as a white solid which was used for the next step without further purification. ¹H NMR (400 MHz, DMSO-d₆): δ1.39 (s, 9H), 1.53-1.65 (m, 2H), 1.85-1.95 (m, 2H), 3.10-3.20 (m, 2H), 3.19 (s, 3H), 3.55-3.65 (m, 2H), 4.80-4.90 (s, 1H).

Step-7 Synthesis of tert-butyl 4-phenoxypiperidine-1-carboxylate (10): To a solution of phenol (2.7 g, 9.67 mmol) in dry DMF (27 mL), Cs₂CO₃ (6.2 g, 19.35 mmol) was added. The reaction mixture was stirred at room temperature for 10 min and 9 (2.7 g, 9.67 mmol) was added to it. The resulting mixture was stirred for 16 h. After completion (monitored by TLC), water (50 mL) was added to the reaction mixture and the aqueous layer was extracted with ethyl acetate (2×80 mL). The organic layers were combined, dried (Na₂SO₄) and concentrated in vacuo. The residue was purified by flash column chromatography [normal phase, silica gel (100-200 mesh), gradient 30% to 40% EtOAc in hexane] to give the title compound (10, 2.3 g, 88%) as a solid. ¹H NMR (400 MHz, DMSO-d₆): δ1.40 (s, 9H), 1.45-1.55 (m, 2H), 1.85-1.92 (m, 2H), 3.12-3.22 (m, 2H), 3.60-3.70 (m, 2H), 4.50-4.60 (s, 1H), 6.72-7.80 (m, 1H), 6.90-7.00 (m, 2H), 7.25-7.30 (m, 2H).

Step-8 Synthesis of 4-phenoxypiperidine (Int. 6): To a solution of 10 (2.2 g, 7.9 mmol) in 1,4-dioxane (20 mL), 4 M HCl in dioxane (10 mL) was added. After completion (monitored by TLC), the reaction mixture was concentrated in vacuo to give the title compound (Int. 6, 1.6 g crude) as a solid. ¹H NMR (400 MHz, DMSO-d₆): δ1.80-1.90 (m, 2H), 2.05-2.15 (m, 2H), 3.00-3.10 (m, 2H), 3.18-3.25 (m, 2H), 4.60-4.70 (s, 1H), 6.90-7.00 (m, 3H), 7.28-7.30 (m, 2H), 9.02 (bs, 1H).

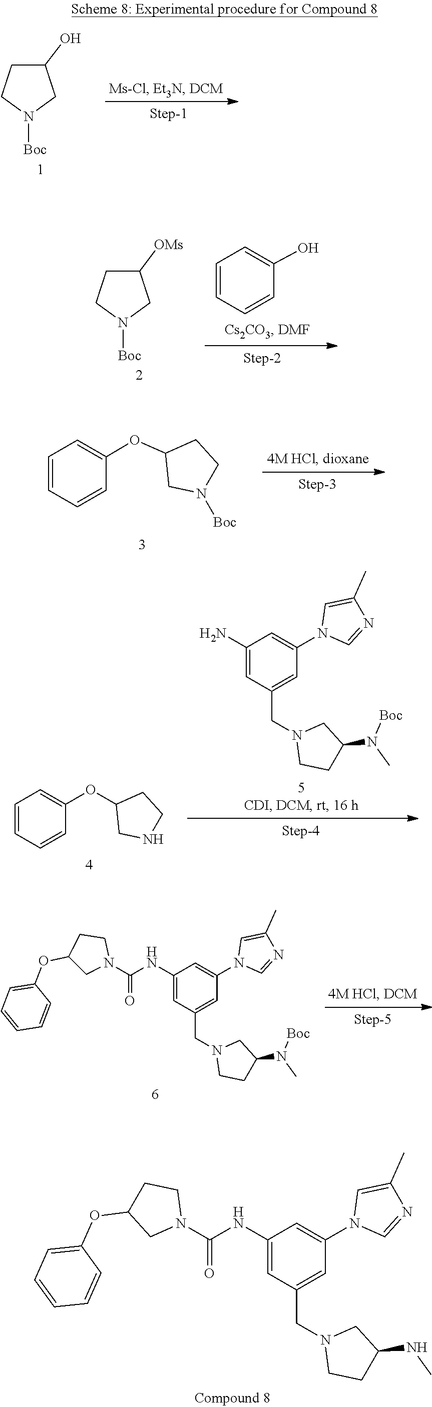

Step-1 Synthesis of tert-butyl 3-((methylsulfonyl)oxy)pyrrolidine-1-carboxylate (2): To a solution of 1 (3.5 g, 17.41 mmol) in DCM (70 mL), Et₃N (2.6 g, 26.1 mmol) was drop wise added at 0° C. After 15 min stirring at same temperature, methane sulfonylchloride (2.3 g, 20.8 mmol) was slowly added at 0° C. The reaction was continued at the room temperature for 1 h. After completion (monitored by TLC), the reaction mixture was quenched with water (70 mL) and compound was extracted with DCM (3×100 mL). The combined organic layer was washed with brine solution (50 mL), dried (Na₂SO₄), concentrated in vacuo to give the title compound (2, 3.6 g, crude) as a white solid which was used for the next step without further purification. LCMS: m/z 264 (M+H)⁺ (ES+), 65%

Step-2 Synthesis of tert-butyl 3-phenoxypyrrolidine-1-carboxylate (3): To the stirred solution of phenol (248 mg, 2.63 mmol) in dry DMF (7 mL), Cs₂CO₃ (1.7 g, 5.27 mmol) was added. The reaction mass was stirred for 10 min and 2 (700 mg, 2.63 mmol) was added at room temperature. The resulting mixture was stirred for 16 h. After completion (monitored by TLC), water (60 mL) was added to the reaction mixture and the aqueous layer was extracted with ethyl acetate (2×30 mL). The combined organic layer was dried (Na₂SO₄) and concentrated in vacuo. The residue was purified by flash column chromatography [normal phase, silica gel (100-200 mesh), gradient 30% to 40% EtOAc in hexane] to give the title compound (3, 600 mg, 86%) as a solid. LCMS: m/z 264 (M+H)⁺ (ES+), 92%

Step-3 Synthesis of 3-phenoxypyrrolidin (4): To a solution of 3 (600 mg, 2.28 mmol) in 1,4-dioxane (10 mL), 4 M HCl in dioxane (5 mL) was added. After completion (monitored by TLC), the reaction mixture was concentrated in vacuo to give 3-phenoxypyrrolidin (4, 250 mg, 67%) as an oily mass. LCMS: m/z 164.12 (M+H)⁺ (ES+), 91%

Step-4 Synthesis of tert-butylmethyl((3S)-1-(3-(4-methyl-1H-imidazol-1-yl)-5-(3-phenoxypyrrolidine-1-carboxamido)benzyl)pyrrolidin-3-yl)carbamate (6): To a solution of 5 (300 mg, 0.77 mmol) in DCM, CDI (378 mg, 2.33 mmol) was added to it and the reaction mixture was stirred at room temperature for 1 h. Then, 3-phenoxypyrrolidine (4, 190 mg, 1.16 mmol) was added and the resulting reaction mixture was stirred at room temperature for 16 h. After completion of reaction (monitored by TLC), the reaction was quenched with water (20 mL) and extracted with DCM (2×20 mL). The combined organic layer was dried (Na₂SO₄) and concentrated in vacuo to give the title compound (6, 430 mg crude) as an oily mass. LCMS: m/z 575.49 (M+H)⁺ (ES+), 70%

Step-5 Synthesis of N-(3-(4-methyl-1H-imidazol-1-yl)-5-(((S)-3-(methylamino)pyrrolidin-1-yl)methyl) phenyl)-3-phenoxypyrrolidine-1-carboxamide (Compound 8): To a solution of 6 (430 mg crude) in 1,4-dioxane (5 mL), 4 M HCl in dioxane (5 mL) was added. After completion (monitored by TLC), the reaction mixture was concentrated in vacuo. The crude material was purified by prep-HPLC (reverse phase, X Terra C18 (19×250) mm, 10 μm, gradient 40% to 60% ACN in water containing 5 mM Ammonium acetate, RT: 11.19 min to give the title compound (Compound 8, 90 mg) as an off-white solid.

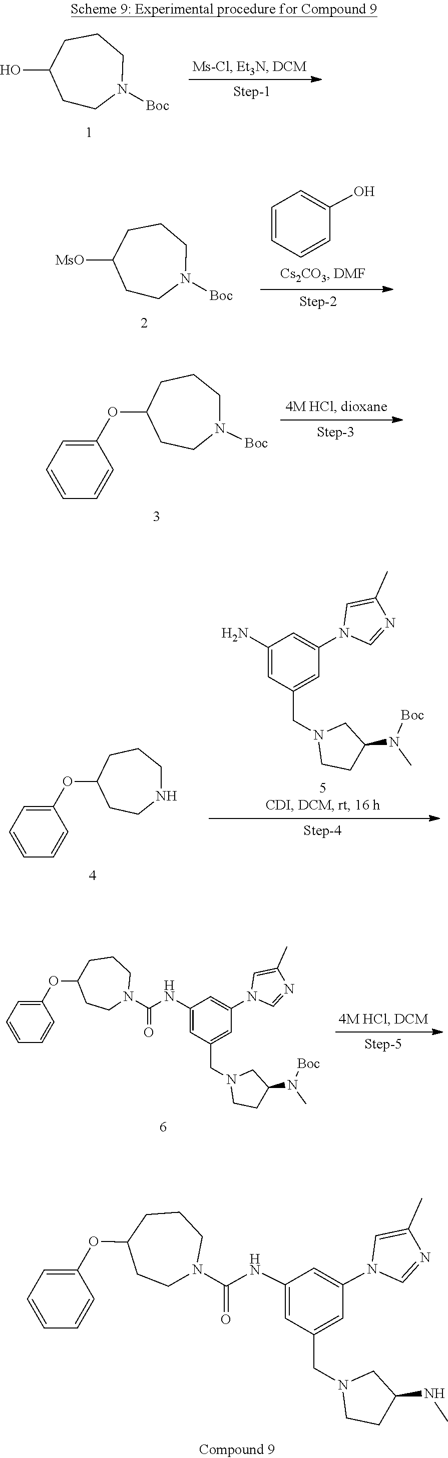

Step-1 Synthesis of tert-butyl 4-((methylsulfonyl)oxy)azepane-1-carboxylate (2): To a solution of 1 (2.5 g, 11.6 mmol) in DCM (50 mL), Et₃N (1.7 g, 17.4 mmol) was drop wise added at 0° C. After 15 min stirring at same temperature, methane sulfonylchloride (1.5 g, 22.3 mmol) was slowly added at 0° C. and the reaction was continued at the room temperature for 1 h. After completion (monitored by TLC), the reaction mixture was quenched with water (70 mL) and compound was extracted with DCM (3×100 mL). The organic layer was washed with brine solution (50 mL), dried (Na₂SO₄), concentrated in vacuo to give the title compound (2, 2.0 g, crude) as an oil which was used for the next step without further purification.

Step-2 Synthesis of tert-butyl 4-phenoxyazepane-1-carboxylate (3): To a solution of phenol (641 mg, 6.82 mmol) in dry DMF (10 mL), Cs₂CO₃ (4.4 g, 13.64 mmol) was added. The reaction mass was stirred for 10 min and 2 (2.0 g, 6.82 mmol) was added at room temperature. The resulting mixture was stirred for 16 h. After completion (monitored by TLC), water (40 mL) was added to the reaction mixture and the aqueous layer was extracted with ethyl acetate (2×60 mL). The organic layers were combined, dried (Na₂SO₄) and concentrated in vacuo. The residue was purified by flash column chromatography [normal phase, silica gel (100-200 mesh), gradient 30% to 40% EtOAc in hexane] to give the title compound (3, 1.2 g, 63%) as a solid. LCMS: m/z 264 (M+H)⁺ (ES+), 92%

Step-3 Synthesis of 4-phenoxyazepane (4): To a solution of 3 (800 mg, 2.74 mmol) in 1,4-dioxane (10 mL), 4 M HCl in dioxane (5 mL) was added. After completion (monitored by TLC), the reaction mixture was concentrated in vacuo to give the title compound (4, 450 g, 67%) as a solid. LCMS: m/z 192.13 (M+H)⁺ (ES+), 99%

Step-4 Synthesis of tert-butyl methyl((3S)-1-(3-(4-methyl-1H-imidazol-1-yl)-5-(4-phenoxyazepane-1-carboxamido)benzyl)pyrrolidin-3-yl)carbamate (6): To a solution of 5 (250 mg, 0.77 mmol) in DCM, CDI (157 mg, 0.974 mmol) was added to it and the reaction mixture was stirred at room temperature for 1 h. Then 4-phenoxyazepane (4, 136 mg, 0.97 mmol) was added and the resulting reaction mixture was stirred at room temperature for 16 h. After completion of reaction (monitored by TLC), the reaction was quenched with water (20 mL) and extracted with DCM (2×40 mL). The combined organic layer was dried (Na₂SO₄) and concentrated in vacuo to give the title compound (6, 350 mg crude) as an oily mass. LCMS: m/z 603.53 (M+H)⁺ (ES+), 66.54%

Step-5 Synthesis of N-(3-(4-methyl-1H-imidazol-1-yl)-5-(((S)-3-(methylamino)pyrrolidin-1-yl)methyl)phenyl)-4-phenoxyazepane-1-carboxamide (Compound 9): To a solution of 6 (320 mg, 0.53 mmol) in 1,4-dioxane (15 mL), 4 M HCl in dioxane (5 mL) was added. After completion (monitored by TLC), the reaction mixture was concentrated in vacuo. The crude material was purified by prep-HPLC (reverse phase, X Terra C18 (19×250) mm, 10 μm, gradient 38% to 62% ACN in water containing 5 mM Ammonium acetate, RT: 17.28 min to give the title compound (Compound 9, 90 mg) as an off-white solid.

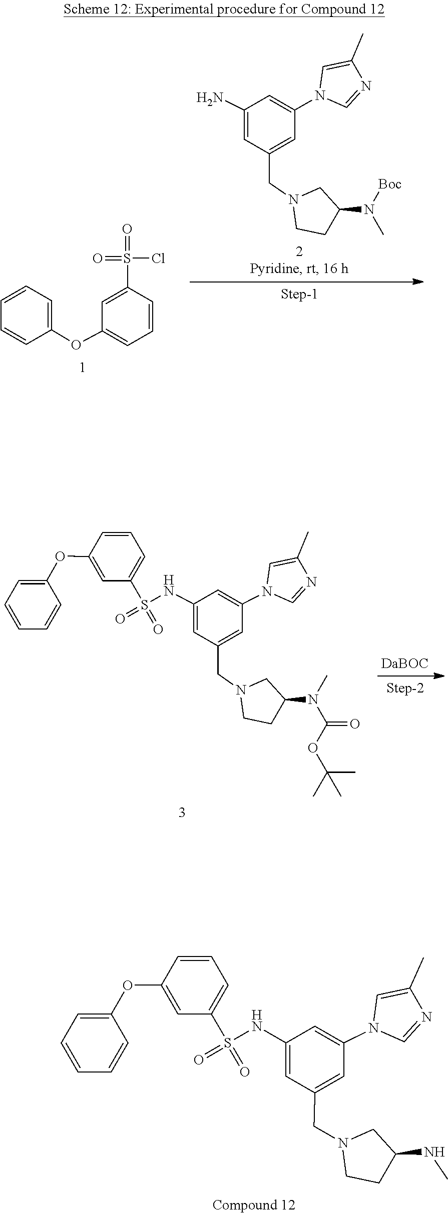 [SMILES: O=S(=O)(Cl)c1cccc(Oc2ccccc2)c1 ; Cc1cn(-c2cc(N)cc(CN3CC[C@H](N(C)C(=O)OC(C)(C)C)C3)c2)cn1 ; Cc1cn(-c2cc(CN3CC[C@H](N(C)C(=O)OC(C)(C)C)C3)cc(NS(=O)(=O)c3cccc(Oc4ccccc4)c3)c2)cn1 ; CN[C@H]1CCN(Cc2cc(NS(=O)(=O)c3cccc(Oc4ccccc4)c3)cc(-n3cnc(C)c3)c2)C1]

Step-1 Synthesis of tert-butyl (S)-methyl(1-(3-(4-methyl-1H-imidazol-1-yl)-5-((3-phenoxyphenyl) sulfonamido)benzyl)pyrrolidin-3-yl)carbamate (3): To a solution of amine 2 (200 mg, 0.52 mmol) in pyridine (5 mL), 3-phenoxybenzenesulfonyl chloride (1, 139 mg, 0.51) was added. The reaction mixture was stirred at room temperature for 16 h. After completion (monitored by TLC), the reaction mixture was concentrated and the residue was dissolved in water. The aqueous layer was washed with DCM (2×30 mL) and the organic layer was dried (Na₂SO₄), concentrated in vacuo to give the title compound (3, 200 mg, crude) as brown solid. LCMS: m/z 618 (M+H)⁺ (ES+), 67%

Step-2 Synthesis of (S)—N-(3-(4-methyl-1H-imidazol-1-yl)-5-((3-(methylamino)pyrrolidin-1-yl)methyl)phenyl)-3-phenoxybenzenesulfonamide (Compound 12): To a solution 3 (200 mg, crude) in dioxane (5 mL), 4 M HCl in dioxane (2 mL) was added and stirred at room temperature for 1 h. After completion (monitored by TLC), the reaction mixture was concentrated in vacuo. The crude material was purified by prep-HPLC (reverse phase, X Bridge Shield C18 (19×250) mm, 10 μm, gradient 22% to 78% ACN in water containing 5 mM Ammonium acetate, RT: 14.73 min to give the title compound as a TFA salt (Compound 12, 40 mg, 26%) as an off-white solid.

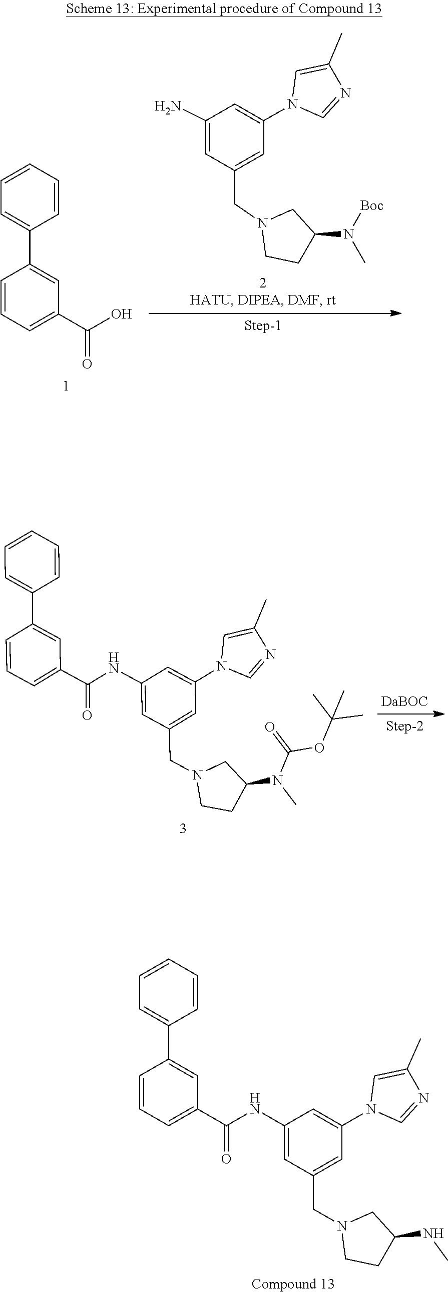 [SMILES: O=C(O)c1cccc(-c2ccccc2)c1 ; Cc1cn(-c2cc(N)cc(CN3CC[C@H](N(C)C(=O)OC(C)(C)C)C3)c2)cn1 ; Cc1cn(-c2cc(CN3CC[C@H](N(C)C(=O)OC(C)(C)C)C3)cc(NC(=O)c3cccc(-c4ccccc4)c3)c2)cn1 ; CN[C@H]1CCN(Cc2cc(NC(=O)c3cccc(-c4ccccc4)c3)cc(-n3cnc(C)c3)c2)C1]

Step-1 Synthesis of tert-butyl (S)-(1-(3-([1,1′-biphenyl]-3-carboxamido)-5-(4-methyl-1H-imidazol-1-yl)benzyl)pyrrolidin-3-yl)(methyl)carbamate (3): To a solution of amine 2 (194 mg, 0.50 mmol) and acid 1 (100 mg, 0.50 mmol) in DMF (5 mL), DIPEA (418 mg, 0.707 mmol) was added followed by addition of HATU (690 mg, 1.16 mmol) at 0° C. The reaction mixture was stirred the at room temperature for 16 h. Progress of the reaction was monitored by TLC, which showed consumption of starting material. After completion of reaction, the reaction mixture was diluted with water (50 mL) and extracted with EtOAc (2×20 mL), the resulting organic layers was dried (Na₂SO₄) and concentrated in vacuo to give the title compound (3, 160 mg, 56%) as a yellow solid. LCMS: m/z 566 (M+H)⁺ (ES+), 81%

Step-2 Synthesis of (S)—N-(3-(4-methyl-1H-imidazol-1-yl)-5-((3-(methylamino)pyrrolidin-1-yl)methyl) phenyl)-[1,1′-biphenyl]-3-carboxamide (Compound 13): To a solution 3 (120 mg) in dioxane 4 mL), 4 M HCl in dioxane (2 mL) was added and stirred at room temperature for 2 h. After completion (monitored by TLC), the reaction mixture was concentrated in vacuo. The crude material was purified by prep-HPLC (reverse phase, X Bridge C18 (19×250) mm, 10 μm, gradient 28% to 72% ACN in water containing 0.1% TFA, RT: 11.14 min to give the title compound as the TFA salt (Compound 13, 40 mg, 40%) as an off-white solid.

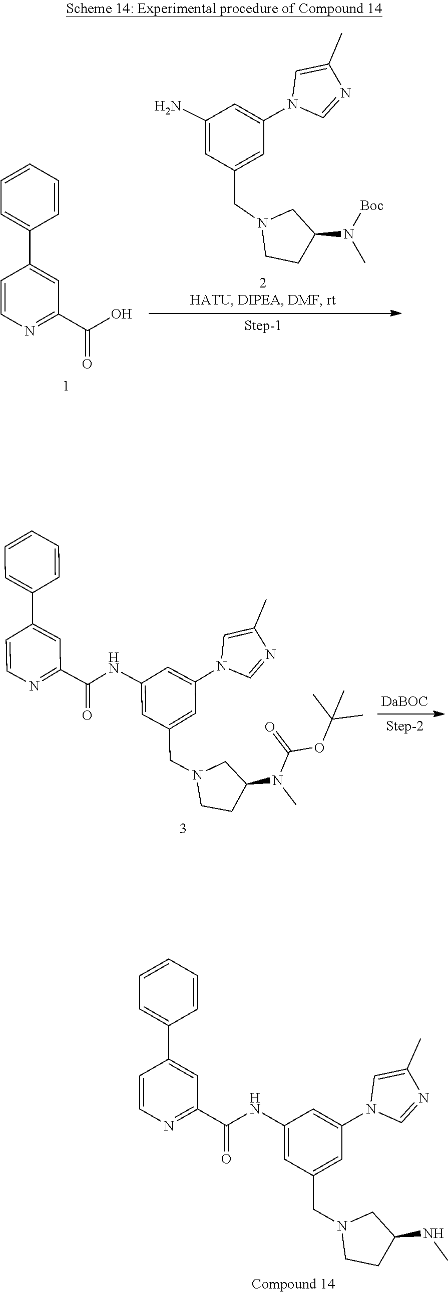 [SMILES: O=C(O)c1cc(-c2ccccc2)ccn1 ; Cc1cn(-c2cc(N)cc(CN3CC[C@H](N(C)C(=O)OC(C)(C)C)C3)c2)cn1 ; Cc1cn(-c2cc(CN3CC[C@H](N(C)C(=O)OC(C)(C)C)C3)cc(NC(=O)c3cc(-c4ccccc4)ccn3)c2)cn1 ; CN[C@H]1CCN(Cc2cc(NC(=O)c3cc(-c4ccccc4)ccn3)cc(-n3cnc(C)c3)c2)C1]

Step-1 Synthesis of tert-butyl (S)-methyl(1-(3-(4-methyl-1H-imidazol-1-yl)-5-(4-phenylpicolinamido)benzyl)pyrrolidin-3-yl)carbamate (3): To a solution of amine 2 (194 mg, 0.75 mmol) and 1 (150 mg, 0.75 mmol) in DMF (5 mL), DIPEA (243 mg, 1.88 mmol) was added followed by addition of HATU (243 mg, 0.90 mmol) at 0° C. The reaction mixture was stirred the at room temperature for 16 h. Progress of the reaction was monitored by TLC, which showed consumption of starting material. After completion of reaction, the reaction mixture was diluted with water (50 mL) and extracted with EtOAc (2×20 mL). The combined organic layers was dried (Na₂SO₄) and concentrated in vacuo to give the title compound (3,280 mg crude) as an off-white solid. LCMS: m/z 567 (M+H)⁺ (ES+), 54.36%

Step-2 Synthesis of (S)—N-(3-(4-methyl-1H-imidazol-1-yl)-5-((3-(methylamino)pyrrolidin-1-yl)methyl) phenyl)-4-phenylpicolinamide (Compound 14): To a solution 3 (250 mg crude) in dioxane (4 mL), 4 M HCl in dioxane (2 mL) was added and stirred at room temperature for 2 h. After completion (monitored by TLC), the reaction mixture was concentrated in vacuo. The crude material was purified by prep-HPLC (reverse phase, X Bridge C18 (19×250) mm, 10 μm, gradient 34% to 66% ACN in water containing 0.1% TFA, RT: 12.15 min to give the title compound (Compound 14, 55 mg, 92%) as an off-white solid.

Structures of Compounds 19, 22, 28, 32, 33, 34, 47, 85 and 94:

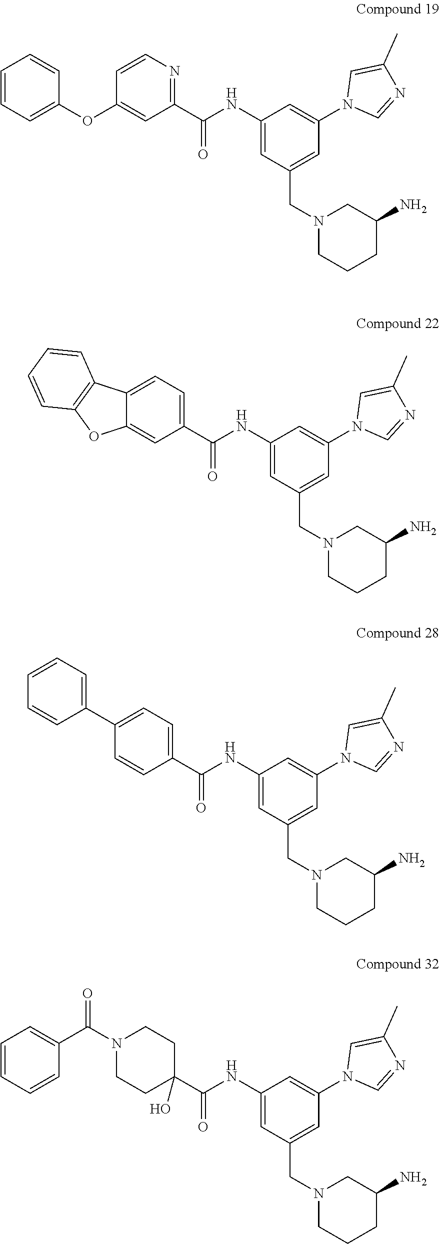 [SMILES: Cc1cn(-c2cc(CN3CCC[C@H](N)C3)cc(NC(=O)c3cc(Oc4ccccc4)ccn3)c2)cn1 ; Cc1cn(-c2cc(CN3CCC[C@H](N)C3)cc(NC(=O)c3ccc4c(c3)oc3ccccc34)c2)cn1 ; Cc1cn(-c2cc(CN3CCC[C@H](N)C3)cc(NC(=O)c3ccc(-c4ccccc4)cc3)c2)cn1 ; Cc1cn(-c2cc(CN3CCC[C@H](N)C3)cc(NC(=O)C3(O)CCN(C(=O)c4ccccc4)CC3)c2)cn1]

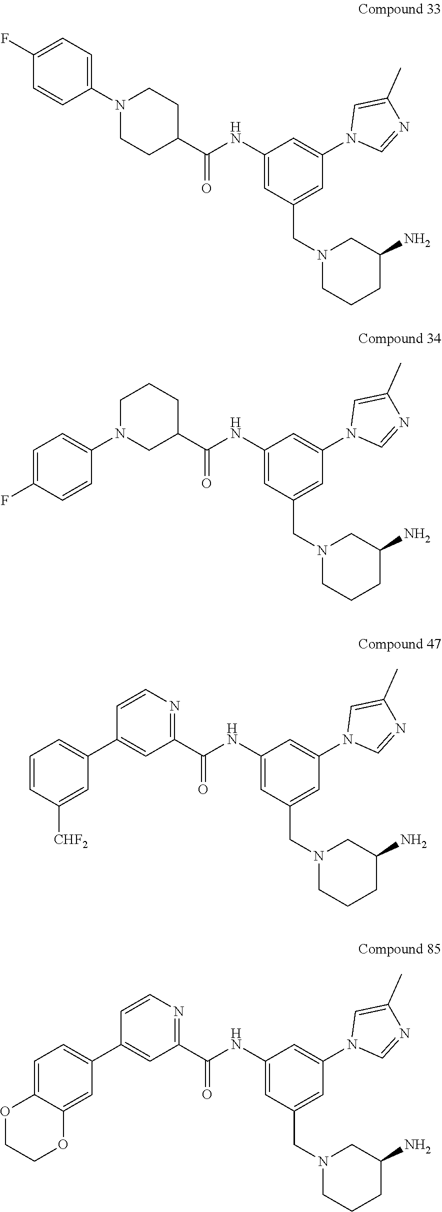 [SMILES: Cc1cn(-c2cc(CN3CCC[C@H](N)C3)cc(NC(=O)C3CCN(c4ccc(F)cc4)CC3)c2)cn1 ; Cc1cn(-c2cc(CN3CCC[C@H](N)C3)cc(NC(=O)C3CCCN(c4ccc(F)cc4)C3)c2)cn1 ; Cc1cn(-c2cc(CN3CCC[C@H](N)C3)cc(NC(=O)c3cc(-c4cccc(C(F)F)c4)ccn3)c2)cn1 ; Cc1cn(-c2cc(CN3CCC[C@H](N)C3)cc(NC(=O)c3cc(-c4ccc5c(c4)OCCO5)ccn3)c2)cn1]

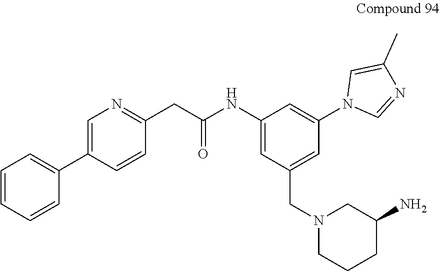 [SMILES: Cc1cn(-c2cc(CN3CCC[C@H](N)C3)cc(NC(=O)Cc3ccc(-c4ccccc4)cn3)c2)cn1]

Experimental Procedure for Compound 19

Step 1 Synthesis of tert-butyl (S)-(1-(3-(4-methyl-1H-imidazol-1-yl)-5-(4-phenoxypicolinamido)benzyl) piperidin-3-yl)carbamate (2b): To a solution of the acid 1b (200 mg, 0.93 mmol) in dry DMF (3 mL), DIPEA (0.29 mL, 2.32 mmol) and HATU (530 mg, 1.39 mmol) was added, followed by the stirring of 10 min and Int. 8 (286 mg, 0.74 mmol) was added at 0° C. The reaction mixture was stirred at room temperature for 16 h. After completion of the reaction (monitored by TLC/LCMS), the reaction mixture was diluted with water (50 mL) and extracted with EtOAc (2×50 mL), the combined organic layer was dried (Na₂SO₄) and concentrated in vacuo. The residue was purified by flash column chromatography [normal phase, silica gel (100-200 mesh), gradient 30% to 40% EtOAc in Hexane] to enrich the purity up to 44% of the title compound (2b, 250 mg) as an off-white semi-solid. LCMS: m/z 583.60 (M+H)⁺ (ES+), 44.63%

Step-2 Synthesis of (S)—N-(3-((3-aminopiperidin-1-yl)methyl)-5-(4-methyl-1H-imidazol-1-yl)phenyl)-4-phenoxypicolinamide (Compound 19): To a solution of corresponding amide 2b (250 mg, 0.43 mmol) in DCM (5 mL), HCl in dioxane (10 mL, 4M) was added at 0° C. and the reaction mass was stirred at room temperature for 2 h. The reaction progress was monitored by TLC, which showed consumption of starting material. After completion of the reaction, the reaction mixture was concentrated in vacuo. The crude material was purified by prep-HPLC (reverse phase, Sunfire C18 (19×250) mm, 10 μm, gradient 16% to 84% ACN in water containing 5 mM Ammonium acetate, RT: 15.16 min to give the title compound (Compound 19, 13 mg, 3% over 2 steps) as an off-white solid.

Experimental Procedure for Compound 22

Step 1 Synthesis of tert-butyl (S)-(1-(3-(dibenzo[b,d]furan-3-carboxamido)-5-(4-methyl-1H-imidazol-1-yl)benzyl)piperidin-3-yl)carbamate (2c): To a solution of the acid 1c (200 mg, 0.94 mmol) in dry DMF (3 mL), DIPEA (0.29 mL, 2.35 mmol) and HATU (537 mg, 1.41 mmol) was added, followed by the stirring of 10 min and Int. 8 (360 mg, 0.74 mmol) was added at 0° C. The reaction mixture was stirred at room temperature for 16 h. After completion of the reaction (monitored by TLC/LCMS), the reaction mixture was diluted with water (50 mL) and extracted with EtOAc (2×50 mL), the combined organic layer was dried (Na₂SO₄) and concentrated in vacuo. The residue was purified by flash column chromatography [normal phase, silica gel (100-200 mesh), gradient 30% to 40% EtOAc in Hexane] to give the title compound (2c, 400 mg) as an off-white semi-solid which was used for further reaction without analysis.

Step-2 Synthesis of (S)—N-(3-((3-aminopiperidin-1-yl)methyl)-5-(4-methyl-1H-imidazol-1-yl)phenyl) dibenzo[b,d]furan-3-carboxamide (Compound 22): To a solution of corresponding amide 2c (400 mg, 0.7 mmol) in DCM (10 mL), HCl in dioxane (7 mL, 4M) was added at 0° C. and the reaction mass was stirred at room temperature for 2 h. The reaction progress was monitored by TLC, which showed consumption of starting material. After completion of the reaction, the reaction mixture was concentrated in vacuo. The crude material was purified by prep-HPLC (reverse phase, X Bridge shield C18 (19×250) mm, 10 μm, gradient 30% to 70% ACN in water containing 5 mM Ammonium bicarbonate, RT: 13.17 min to give the title compound (Compound 22, 70 mg, 17% over 2 steps) as an off-white solid.

Experimental Procedure for Compound 28

Step 1 Synthesis of tert-butyl (S)-(1-(3-([1,1′-biphenyl]-4-carboxamido)-5-(4-methyl-1H-imidazol-1-yl)benzyl)piperidin-3-yl)carbamate (2e): To a solution of the acid 1e (300 mg, 1.30 mmol) in dry DMF (3 mL), DIPEA (0.56 mL, 2.32 mmol) and HATU (741 mg, 1.95 mmol) was added, followed by the stirring of 10 min and Int. 8 (401 mg, 1.03 mmol) was added at 0° C. The reaction mixture was stirred at room temperature for 16 h. After completion of the reaction (monitored by TLC/LCMS), the reaction mixture was diluted with water (50 mL) and extracted with EtOAc (2×50 mL), the combined organic layer was dried (Na₂SO₄) and concentrated in vacuo. The residue was purified by flash column chromatography [normal phase, silica gel (100-200 mesh), gradient 30% to 40% EtOAc in Hexane] to enrich 68% purity of the title compound (2e, 600 mg) as brown solid. LCMS: m/z 564.60 (M+H)⁺ (ES+), 67.76%

Step-2 Synthesis of (S)—N-(3-((3-aminopiperidin-1-yl)methyl)-5-(4-methyl-1H-imidazol-1-yl)phenyl)-[1,1′-biphenyl]-4-carboxamide (Compound 28): To a solution of corresponding amide 2e (300 mg, 0.53 mmol) in DCM (10 mL), HCl in dioxane (10 mL, 4M) was added at 0° C. and the reaction mass was stirred at room temperature for 2 h. The reaction progress was monitored by TLC, which showed consumption of starting material. After completion of the reaction, the reaction mixture was concentrated in vacuo. The crude material was purified by prep-HPLC (reverse phase, Sunfire C18 (19×250) mm, 10 μm, gradient 40% to 80% ACN in water containing 0.1% TFA, RT: 11.19 min to give the title compound (Compound 28, 93 mg, 26% over 2 steps) as an off-white solid.

Experimental Procedure for Compound 32

Step 1 Synthesis of tert-butyl (S)-(1-(3-(1-benzoyl-4-hydroxypiperidine-4-carboxamido)-5-(4-methyl-1H-imidazol-1-yl)benzyl)piperidin-3-yl)carbamate (2f): To a solution of the acid 1f (280 mg, 0.538 mmol) in dry DMF (3 mL), DIPEA (0.56 mL, 2.32 mmol) and HATU (307 mg, 0.538 mmol) was added, followed by the stirring of 10 min and Int. 8 (207 mg, 0.538 mmol) was added at 0° C. The reaction mixture was stirred at room temperature for 16 h. After completion of the reaction (monitored by TLC/LCMS), the reaction mixture was diluted with water (50 mL) and extracted with EtOAc (2×50 mL), the combined organic layer was dried (Na₂SO₄) and concentrated in vacuo. The residue was purified by flash column chromatography [normal phase, silica gel (100-200 mesh), gradient 30% to 40% EtOAc in Hexane] to give the title compound (2f, 120 mg, 37.79%) as a brown solid. LCMS: m/z 591.63 (M+H)⁺ (ES+), 68.22%

Step-2 Synthesis of (S)—N-(3-((3-aminopiperidin-1-yl)methyl)-5-(4-methyl-1H-imidazol-1-yl)phenyl)-1-benzoyl-4-hydroxypiperidine-4-carboxamide (Compound 32): To a solution of corresponding amide 2f (280 mg, 0.31 mmol) in DCM (20 mL), HCl in dioxane (10 mL, 4M) was added at 0° C. and the reaction mass was stirred at room temperature for 2 h. The reaction progress was monitored by TLC, which showed consumption of starting material. After completion of the reaction, the reaction mixture was concentrated in vacuo. The crude material was purified by prep-HPLC (reverse phase, X Bridge shield C₈ (19×250) mm, 10 μm, gradient 30% to 70% ACN in water containing 5 mM Ammonium acetate, RT: 12.39 min to give the title compound (Compound 32, 25 mg, 5.7% over 2 steps) as an off-white solid.

Experimental Procedure for Compound 33

Step 1 Synthesis of tert-butyl (S)-(1-(3-(1-(4-fluorophenyl)piperidine-4-carboxamido)-5-(4-methyl-1H-imidazol-1-yl)benzyl)piperidin-3-yl)carbamate (2g): To a solution of the acid 1g (200 mg, 0.89 mmol) in dry DMF (3 mL), DIPEA (0.39 mL, 2.24 mmol) and HATU (686 mg, 1.34 mmol) was added, followed by the stirring of 10 min and Int. 8 (276 mg, 0.71 mmol) was added at 0° C. The reaction mixture was stirred at room temperature for 16 h. After completion of the reaction (monitored by TLC/LCMS), the reaction mixture was diluted with water (50 mL) and extracted with EtOAc (2×50 mL), the combined organic layer was dried (Na₂SO₄) and concentrated in vacuo. The residue was purified by flash column chromatography [normal phase, silica gel (100-200 mesh), gradient 30% to 40% EtOAc in Hexane] to enrich the purity up to 46% of the title compound (2g, 420 mg) as a brown solid. LCMS: m/z 589.29 (M−H)⁻ (ES−), 46.62%

Step-2 Synthesis of (S)—N-(3-((3-aminopiperidin-1-yl)methyl)-5-(4-methyl-1H-imidazol-1-yl)phenyl)-1-(4-fluorophenyl)piperidine-4-carboxamide (Compound 33): To a solution of corresponding amide 2g (420 mg, 0.71 mmol) in DCM (10 mL), HCl in dioxane (15 mL, 4M) was added at 0° C. and the reaction mass was stirred at room temperature for 2 h. The reaction progress was monitored by TLC, which showed consumption of starting material. After completion of the reaction, the reaction mixture was concentrated in vacuo. The crude material was purified by prep-HPLC (reverse phase, X Bridge shield C18 (19×250) mm, 10 μm, gradient 28% to 72% ACN in water containing 5 mM Ammonium acetate, RT: 14.89 min to give the title compound (Compound 33, 60 mg, 14% over 2 steps) as an off-white solid.

Experimental Procedure for Compound 34

Step 1 Synthesis of tert-butyl ((3S)-1-(3-(1-(4-fluorophenyl)piperidine-3-carboxamido)-5-(4-methyl-1H-imidazol-1-yl)benzyl)piperidin-3-yl)carbamate (2h): To a solution of the acid 1h (200 mg, 0.89 mmol) in dry DMF (10 mL), DIPEA (0.39 mL, 2.24 mmol) and HATU (530 mg, 2.25 mmol) was added, followed by the stirring of 10 min and Int. 8 (276 mg, 0.71 mmol) was added at 0° C. The reaction mixture was stirred at room temperature for 16 h. After completion of the reaction (monitored by TLC/LCMS), the reaction mixture was diluted with water (50 mL) and extracted with EtOAc (2×50 mL), the combined organic layer was dried (Na₂SO₄) and concentrated in vacuo. The residue was purified by flash column chromatography [normal phase, silica gel (100-200 mesh), gradient 30% to 40% EtOAc in Hexane] to enrich the purity up to 63% of the title compound (2h, 300 mg) as brown solid. LCMS: m/z 589.78 (M−H)⁻ (ES−), 63%

Step-2 Synthesis of N-(3-(((S)-3-aminopiperidin-1-yl)methyl)-5-(4-methyl-1H-imidazol-1-yl)phenyl)-1-(4-fluorophenyl)piperidine-3-carboxamide (Compound 34): To a solution of corresponding amide 2h (136 mg, 0.237 mmol) in DCM (5 mL), HCl in dioxane (15 mL, 4M) was added at 0° C. and the reaction mass was stirred at room temperature for 2 h. The reaction progress was monitored by TLC, which showed consumption of starting material. After completion of the reaction, the reaction mixture was concentrated in vacuo. The crude material was purified by prep-HPLC (reverse phase, Symmetry C18 (19×250) mm, 10 μm, gradient 28% to 72% ACN in water containing 5 mM Ammonium acetate, RT: 10.12 min to give the title compound (Compound 34, 8 mg, 2.3% over 2 steps) as a white solid.

Experimental Procedure for Compound 47

Step 1 Synthesis of tert-butyl (S)-(1-(3-(4-(4-(difluoromethyl)phenyl)picolinamido)-5-(4-methyl-1H-imidazol-1-yl)benzyl)piperidin-3-yl)carbamate (2i): To a solution of the acid 1i (270 mg, 1.08 mmol) in dry DMF (3 mL), DIPEA (0.39 mL, 4.33 mmol) and HATU (494 mg, 1.93 mmol) was added, followed by the stirring of 10 min and Int. 8 (209 mg, 0.54 mmol) was added at 0° C. The reaction mixture was stirred at room temperature for 16 h. After completion of the reaction (monitored by TLC/LCMS), water (20 mL) was added to the reaction mixture and the aqueous layer was extracted with ethyl acetate (2×30 mL). The organic layers were combined, dried (Na₂SO₄) and concentrated in vacuo to give the title compound (2i, 100 mg crude) as a brownish solid which was used to next step without purification. LCMS: m/z 617.12 (M+H)⁺ (ES+), 30.44%

Step-2 Synthesis of (S)—N-(3-((3-aminopiperidin-1-yl)methyl)-5-(4-methyl-1H-imidazol-1-yl)phenyl)-4-(3-(difluoromethyl)phenyl)picolinamide (Compound 47): To a solution of corresponding amide 2i (100 mg, 0.16 mmol) in DCM (5 mL), HCl in dioxane (10 mL, 4M) was added at 0° C. and the reaction mass was stirred at room temperature for 2 h. The reaction progress was monitored by TLC, which showed consumption of starting material. After completion of the reaction, the reaction mixture was concentrated in vacuo. The residue was purified by prep-HPLC (reverse phase, T3 C18 (19×250) mm, 10 μm, gradient 37% to 65% ACN in water containing 5 mM Ammonium acetate, RT: 15.10 min to give the title compound (Compound 47, 15 mg, 17.37%, over three steps) as an off-white semi-solid.

Experimental Procedure for Compound 85

Step 1 Synthesis of tert-butyl N-[(3S)-1-({3-[4-(2,3-dihydro-1,4-benzodioxin-6-yl)pyridine-2-amido]-5-(4-methyl-1H-imidazol-1-yl)phenyl}methyl)piperidin-3-yl]carbamate (2j): To a solution of the acid 1j (150 mg, 0.583 mmol) in dry DMF (3 mL), DIPEA (0.30 mL, 1.75 mmol) and HATU (165 mg, 0.70 mmol) was added, followed by the stirring of 10 min and Int. 8 (225 mg, 0.583 mmol) was added at 0° C. The reaction mixture was stirred at room temperature for 16 h. After completion of the reaction (monitored by TLC/LCMS), the reaction mixture was diluted with water (50 mL) and extracted with EtOAc (2×50 mL), the combined organic layer was dried (Na₂SO₄) and concentrated in vacuo. The residue was purified by flash column chromatography [normal phase, silica gel (100-200 mesh), gradient 30% to 40% EtOAc in Hexane] to give the title compound (2j, 275 mg, 66%) as brown solid. LCMS: m/z 625.37 (M+H)⁺ (ES+), 88%

Step-2 Synthesis of (S)—N-(3-((3-aminopiperidin-1-yl)methyl)-5-(4-methyl-1H-imidazol-1-yl)phenyl)-4-(2,3-dihydrobenzo[b][1,4]dioxin-6-yl)picolinamide (Compound 85): To a solution of corresponding amide 2j (220 mg, 0.35 mmol) in DCM (10 mL), HCl in dioxane (10 mL, 4M) was added at 0° C. and the reaction mass was stirred at room temperature for 2 h. The reaction progress was monitored by TLC, which showed consumption of starting material. After completion of the reaction, the reaction mixture was concentrated in vacuo. The residue was purified by prep-HPLC (reverse phase, X bridge shield C18 (19×250) mm, 10 μm, gradient 42% to 58% ACN in water containing 5 mM Ammonium acetate, RT: 9.79 min to give the title compound (Compound 85, 50 mg, 19%) as an off-white solid.

Experimental Procedure for Compound 94

Step 1 Synthesis of tert-butyl N-[(3S)-1-{[3-(4-methyl-1H-imidazol-1-yl)-5-[2-(5-phenylpyridin-2-yl)acetamido]phenyl]methyl}piperidin-3-yl]carbamate (2k): To a solution of the acid 1k (170 mg, 0.598 mmol) in dry DMF (3 mL), DIPEA (0.62 mL, 2.24 mmol) and HATU (718 mg, 1.20 mmol) was added, followed by the stirring of 10 min and Int. 8 (231 mg, 0.598 mmol) was added at 0° C. The reaction mixture was stirred at room temperature for 16 h. After completion of the reaction (monitored by TLC/LCMS), the reaction mixture was diluted with water (50 mL) and extracted with EtOAc (2×50 mL), the combined organic layer was dried (Na₂SO₄) and concentrated in vacuo. The residue was purified by flash column chromatography [normal phase, silica gel (100-200 mesh), gradient 30% to 40% EtOAc in Hexane] to give the title compound (2k, 100 mg, 27%) as brown solid. LCMS: m/z 581 (M+H)⁺ (ES+), 96%

Step-2 Synthesis of (S)—N-(3-((3-aminopiperidin-1-yl)methyl)-5-(4-methyl-1H-imidazol-1-yl)phenyl)-2-(5-phenylpyridin-2-yl)acetamide (Compound 94): To a solution of corresponding amide 2k (100 mg) in DCM (10 mL), HCl in dioxane (4.0 mL, 4M) was added at 0° C. and the reaction mass was stirred at room temperature for 2 h. The reaction progress was monitored by TLC, which showed consumption of starting material. After completion of the reaction, the reaction mixture was concentrated in vacuo. The residue was purified by flash column chromatography [normal phase, silica gel (100-200 mesh), gradient 5% to 10% methanol in DCM] to give the title compound (Compound 94, 50 mg, 59.72%) as an off-white solid.

Corresponding Acid Synthesis 1b, 1f, 1g, 1h, 1i, 1j and 1k

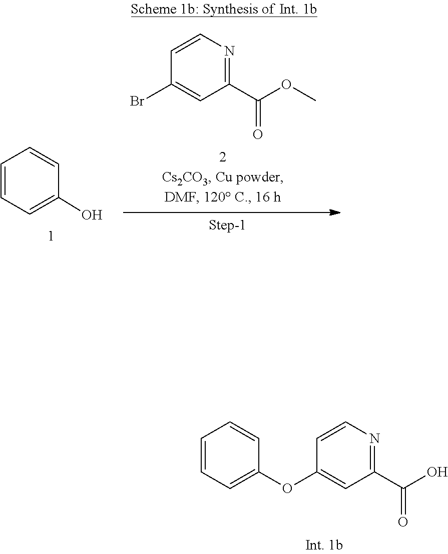 [SMILES: COC(=O)c1cc(Br)ccn1 ; Oc1ccccc1 ; O=C(O)c1cc(Oc2ccccc2)ccn1]

Step-1 Synthesis of 4-phenoxypicolinic acid (1b): A solution of methyl 4-bromopicolinate (2, 0.70 g, 3.25 mmol) and phenol (1, 0.46 g, 4.88 mmol) in DMF (10 mL), Cs₂CO₃ (3.17 g, 9.75 mmol) was added. The reaction vial was sealed with aluminum cap and heated at 175° C. for 48 h. After completion of the reaction (monitored by TLC), the reaction mixture was concentrated and water was added and the aqueous layer was extracted with ethyl acetate (2×30 mL). The organic layers were dried (Na₂SO₄) and concentrated in vacuo to give the title compound (1b, 0.64 g, crude) as brown gummy. LCMS: m/z 214 (M−H)⁻ (ES−), 72%

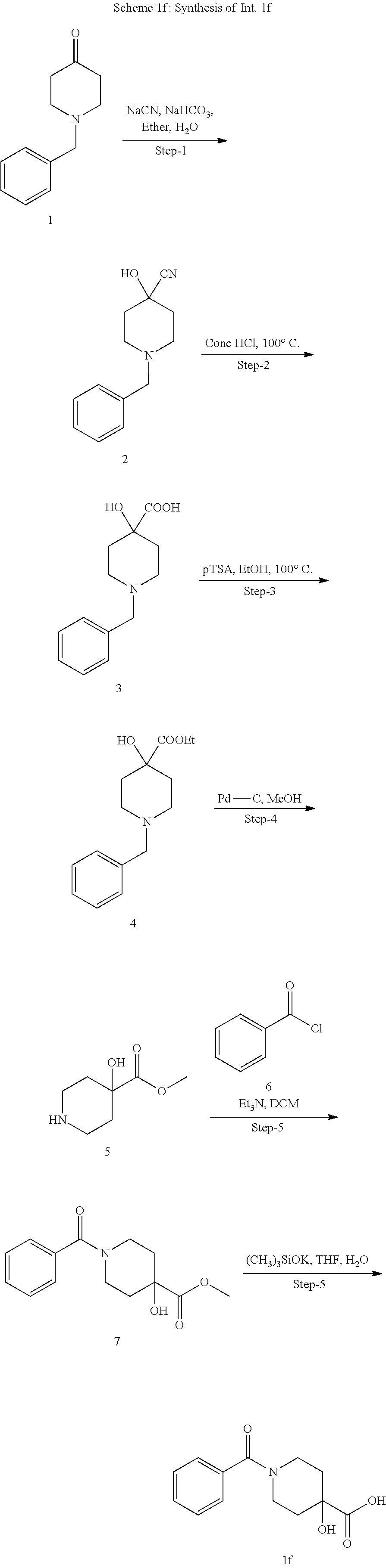

Step-1 Synthesis of 1-benzyl-4-hydroxypiperidine-4-carbonitrile (2): To a mixture of NaCN (3.11 g, 63.4 mmol) and NaHCO₃ (8.88 g, 106 mmol) in H₂O (100 mL), 1-benzylpiperidin-4-one (1, 9.79 mL, 52.8 mmol) in diethyl Ether (200 mL) was added at room temperature. The reaction was stirred at room temperature for 18 h. The Et₂O layer was separated and water layer was extracted with Et₂O (50 mL). The combined organic layer was washed with brine, dried (sodium sulfate) and concentrated in vacuo to give the title compound (2, 8.00 g, crude) as a white solid. LCMS: m/z 217.19 (M+H)⁺ (ES+), 57.51%

Step-2 Synthesis of 1-benzyl-4-hydroxypiperidine-4-carboxylic acid (3): A mixture of 1-benzyl-4-hydroxypiperidine-4-carbonitrile (2, 7.00 g crude) and conc. HCl (50 mL) was stirred at 100° C. for 4 h. After completion of reaction (monitored by TLC & LCMS), the solvent was evaporated in vacuo to give the title compound (3, 7.0 g crude) as a white solid. LCMS: m/z 236.16 (M+H)⁺ (ES+), 83%

Step-3 Synthesis of ethyl 1-benzyl-4-hydroxypiperidine-4-carboxylate (4): To a solution of 1-benzyl-4-hydroxypiperidine-4-carboxylic acid (3, 7.00 g, 28.1 mmol) in EtOH (100 mL), p-TsOH (454 mg) was added and stirred at 105° C. for 5 h. After completion of reaction (monitored by TLC & LCMS), the solvent was evaporated in vacuo and the crude material was dissolved in DCM (200 mL). The organic layer was washed with saturated NaHCO₃ (2×60 mL), dried (sodium sulfate) and evaporated in vacuo to give the title compound (4, 6 g, 77%) as a white solid. LCMS: m/z 264 (M+H)⁺ (ES+), 91%

Step-4 Synthesis of ethyl 4-hydroxypiperidine-4-carboxylate (5): To a stirred solution of ethyl 1-benzyl-4-hydroxypiperidine-4-carboxylate (4, 4.00 g, 15.2 mmol) in MeOH (250 mL), 10% Pd on carbon (1.62 g, 15.2 mmol, 50% wet) was added to it and the reaction mixture was stirred at room temperature for 18 h. After completion of reaction (monitored by TLC), the reaction mixture was filtered on celite bed and thoroughly washed with methanol. All mother liquor was concentrated to give the title compound (5, 2.50 g crude) as a brown solid, which was used for further reaction without purification.

Step-5 Synthesis of methyl 1-benzoyl-4-hydroxypiperidine-4-carboxylate (7): To a stirred solution of ethyl 4-hydroxypiperidine-4-carboxylate (5, 1.31 g crude, 7.54 mmol) in DCM (10.0 mL), Et₃N (1.25 mL, 9.05 mmol) was added followed by addition of Benzoyl chloride (6, 0.657 mL, 5.65 mmol) at 0° C. and the reaction mixture was stirred at room temperature for 18 h. After completion of reaction, the reaction mixture was diluted with water (30 mL) and aqueous layer was extracted with DCM (2×50 mL). The combined organic layer was dried (Na₂SO₄), concentrated in vacuo to give the title compound (7, 1 g, 47%) as a brownish sticky solid. LCMS: m/z 278.22 (M+H)⁺ (ES+), 98%

Step-6 Synthesis of 1-benzoyl-4-hydroxypiperidine-4-carboxylic acid (1f): To a stirred solution of methyl 1-benzoyl-4-hydroxypiperidine-4-carboxylate (7, 500 mg, 1.90 mmol) in THF (10.0 mL) and water (3.00 mL), trimethylsilanol potassium (122 mg, 0.950 μmol) was added. The reaction mixture was stirred at room temperature for 18 h. After completion of the reaction (monitored by TLC), reaction mixture was concentrated and water (20 mL) was added. Aqueous layer was extracted with ether (2×20 mL) and organic layer was separated. Aqueous layer was acidified with aq NaHSO₄ solution (10 mL). The aqueous layer was extracted with 10% MeOH in DCM (2×30 mL). The organic layers were combined, dried (Na₂SO₄) and concentrated in vacuo to give the title compound (1f, 390 mg, 42%) as a brown gum. LCMS: m/z 249.98 (M+H)⁺ (ES+), 98%

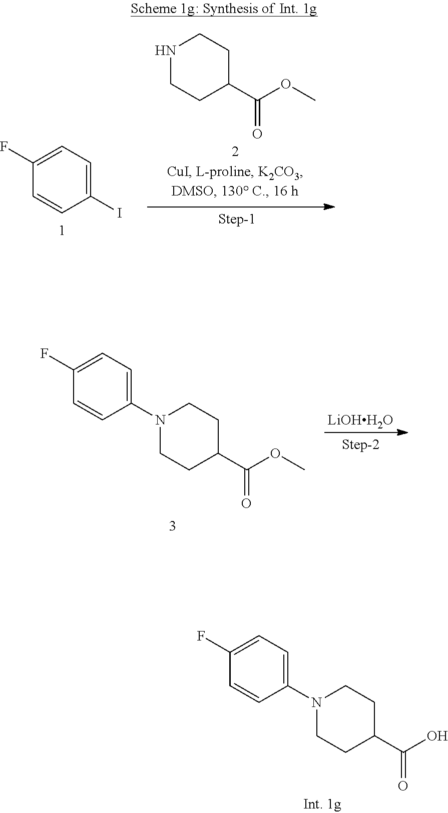

Step-1 Synthesis of methyl 1-(4-fluorophenyl)piperidine-4-carboxylate (3): To a solution of 1-fluoro-4-iodobenzene (1, 2.26 g, 10.19 mmol) in DMSO (10 mL), methyl piperidine-4-carboxylate (2, 1.0 g, 6.36 mmol), L-proline (1.39 g, 12.10 mmol), CuI (0.605 g, 3.18 mmol) and K₂CO₃ (2.9 g, 21.19 mmol) were sequentially added at room temperature under N₂. The reaction mixture was heated at 130° C. for 18 h. After cooling at room temperature, water (30 mL) was added and reaction mixture was filtered through a pad of celite, washed with EtOAc (2×30 mL). The filtrate was extracted with ethyl acetate (2×500 mL). The organic layer washed with brine solution (8×500 mL) and dried (Na₂SO₄), concentrated in vacuo to give crude residue. This obtained residue was triturated by ethyl acetate (3×250 ml) to give the title compound (3, 0.9 g, 56%) as a greenish liquid. LCMS: m/z 238.12 (M+H)⁺ (ES+), 60.26%

Step-2 Synthesis of 1-(4-fluorophenyl)piperidine-4-carboxylic acid (1g): To a stirred solution of (3, 900 mg, 3.58 mmol) in THF (10.0 mL) and water (3.00 mL), LiOH·H₂O (1.5 g, 35.58 mmol) was added. The reaction mixture was stirred at rt for 18 h. After completion of the reaction (monitored by TLC), water (20 mL) was added and aqueous layer was extracted with ether (2×20 mL) and the organic layer was separated. Aqueous layer was acidified with 5% aqueous NaHSO₄ solution (10 mL) and extracted with 10% MeOH in DCM (2×30 mL). The organic layers were combined, dried (Na₂SO₄) and concentrated in vacuo to give the title compound (1g, 265 mg, 33%) as a white solid. LCMS: m/z 222.26 (M−H)⁻ (ES−), 97%

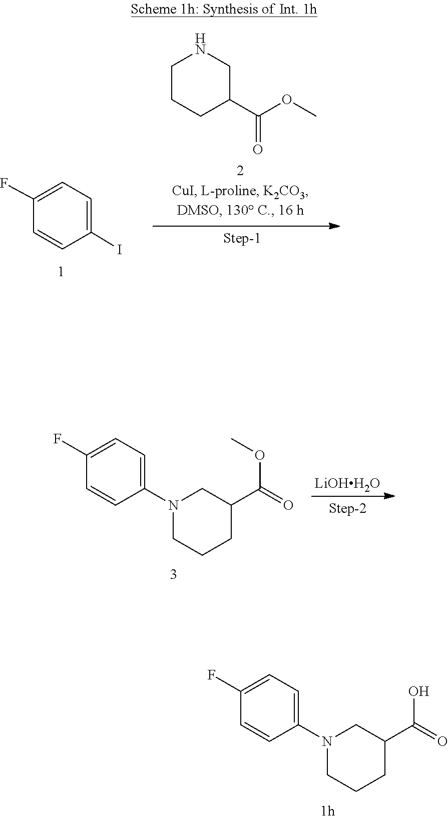

Step-1 Synthesis of methyl 1-(3-fluorophenyl)piperidine-3-carboxylate (3): To a solution of 1-fluoro-4-iodobenzene (1, 1.29 g, 11.2 mmol) in DMSO (10 mL), methyl piperidine-3-carboxylate (2, 0.979 mL, 6.98 mmol), L-proline (1.53 g, 13.3 mmol), CuI (0.665 g, 3.49 mmol) and K₂CO₃ (3.19 g, 23 mmol) were sequentially added at room temperature under N₂. The reaction mixture was heated at 70° C. for 18 h. After cooling at room temperature, water (30 mL) was added and reaction mixture was filtered through a pad of celite, washed with EtOAc (2×30 mL). The filtrate was extracted with ethyl acetate (2×500 mL). The organic layer washed with brine solution (8×500 mL) and dried (Na₂SO₄), concentrated in vacuo to give crude residue. This obtained residue was triturated by ethyl acetate (3×250 ml) to give the title compound (3, 230 mg, 13.8%) as a yellow liquid. LCMS: m/z 238.12 (M+H)⁺ (ES+), 96%

Step-2 Synthesis of 1-(4-fluorophenyl)piperidine-3-carboxylic acid (1h): To a stirred solution of methyl 1-(3-fluorophenyl)piperidine-3-carboxylate (3, 230 mg, 0.969 mmol) in THF (20.0 mL), and aqueous solution of LIOH·H₂O (46.4 mg, 1.94 mmol) in water (7 mL) was added. The reaction mixture was stirred at 60° C. for 18 h. After completion of reaction (monitored by TLC). The reaction mixture was diluted in water and the aqueous layer was extracted with ethyl acetate (2×30 mL). Aqueous layer was acidified with 5% aqueous NaHSO₄ solution (10 mL) and extracted with 10% MeOH in DCM (2×30 mL). The organic layers were combined, dried (Na₂SO₄) and concentrated in vacuo to give the title compound (1h, 200 mg, crude) as a white solid which was used for further reaction without purification.

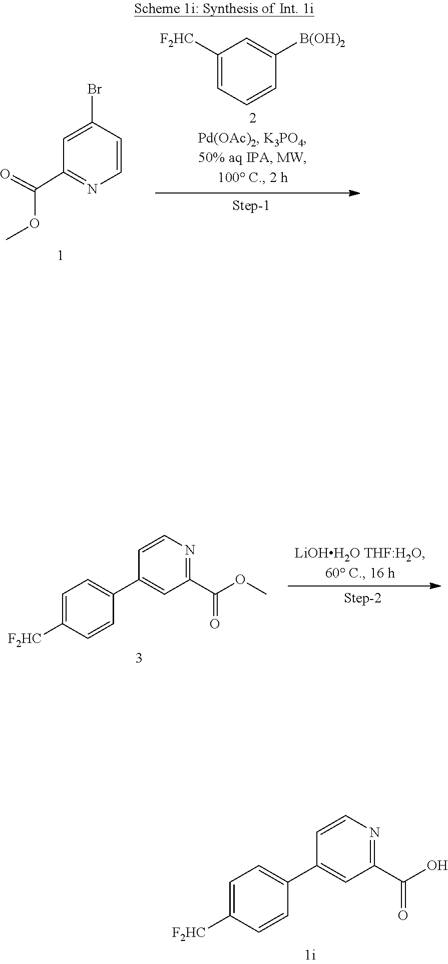 [SMILES: COC(=O)c1cc(Br)ccn1 ; OB(O)c1cccc(C(F)F)c1 ; COC(=O)c1cc(-c2ccc(C(F)F)cc2)ccn1 ; O=C(O)c1cc(-c2ccc(C(F)F)cc2)ccn1]

Step-1 Synthesis of methyl 4-(4-(difluoromethyl)phenyl)picolinate(3): A solution of methyl 4-bromopicolinate (1, 500 mg, 1.97 mmol) and (3-(difluoromethyl)phenyl) boronic acid (2, 425 mg, 1.97 mmol) in) in propan-2-ol and water (10 mL, 1:1) was degassed with argon for 10 min. After degassing, palladium diacetate (44 mg, 0.19 mmol) and tri potassium phosphate (1.25 g, 5.90 mmol) were added. The reaction vial was sealed with aluminum cap and irradiated in microwave at 100° C. for 1 h. After completion of the reaction (monitored by TLC), the reaction was filtered through a pad of celite and washed with IPA (50 mL). Organic layers were combined, dried (Na₂SO₄) and concentrated in vacuo to give crude of the title compound (3, 120 mg crude) as a black liquid. Crude was used for next step without purification. LCMS: m/z 263.86 (M+H)⁺ (ES+), 32.05%

Step-2 Synthesis of 4-(4-(difluoromethyl)phenyl)picolinic acid (1i): To a stirred solution of methyl 4-(4-(difluoromethyl)phenyl)picolinate (3, 100 mg, 0.38 mmol) in THF (10.0 mL) and water (3.00 mL), LiOH·H₂O (313 mg, 7.47 mmol) was added. The reaction mixture was stirred at 60° C. for 18 h. After completion of the reaction (monitored by TLC), reaction mixture was concentrated and water (20 mL) was added. Aqueous layer was extracted with ether (2×20 mL) and organic layer was separated. Aqueous layer was acidified with aq NaHSO₄ solution (10 mL). The aqueous layer was extracted with 10% MeOH in DCM (2×30 mL). The organic layers were combined, dried (Na₂SO₄) and concentrated in vacuo to give the title compound (1i, 300 mg crude) as a brown gum. Crude was used for next step without purification. LCMS: m/z 249.81 (M+H)⁺ (ES+), 34.34%

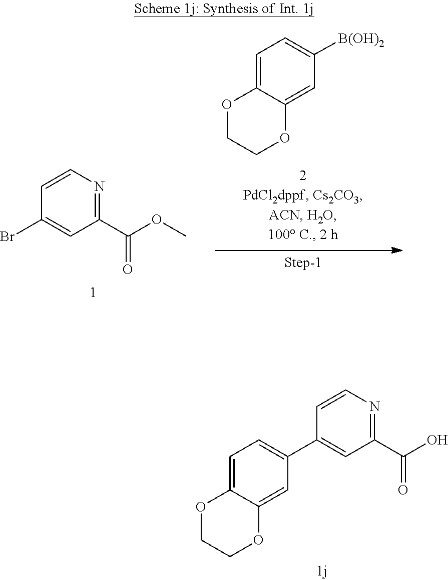 [SMILES: OB(O)c1ccc2c(c1)OCCO2 ; COC(=O)c1cc(Br)ccn1 ; O=C(O)c1cc(-c2ccc3c(c2)OCCO3)ccn1]

Synthesis of 4-(2,3-dihydrobenzo[b][1,4]dioxin-6-yl)picolinic acid (1j): To a stirred solution of methyl 4-bromopicolinate (100 mg, 0.63 mmol) and (2,3-dihydrobenzo[b][1,4]dioxin-6-yl)boronic acid (136 mg, 0.756 mmol) in ACN:H₂O (3 mL, 1:1), Cs₂CO₃ (309 mg, 0.945 mmol) was added and degassed with N₂ for 10 min. PdCl₂(dppf)·DCM (46 mg, 0.063 mmol) was added to it and heated the reaction mixture at 90° C. for 16 h. After completion (monitored by TLC or LCMS), the reaction mixture was diluted with water (50 mL) and extracted with ethyl acetate (2×50 mL). The combined organic was dried (Na₂SO₄) and concentrated in vacuo. The crude was purified by column chromatography by using 10% MeOH in DCM as eluent to give the title compound (1j, 172 mg crude) as an off-white solid which was used to the next step without purification.

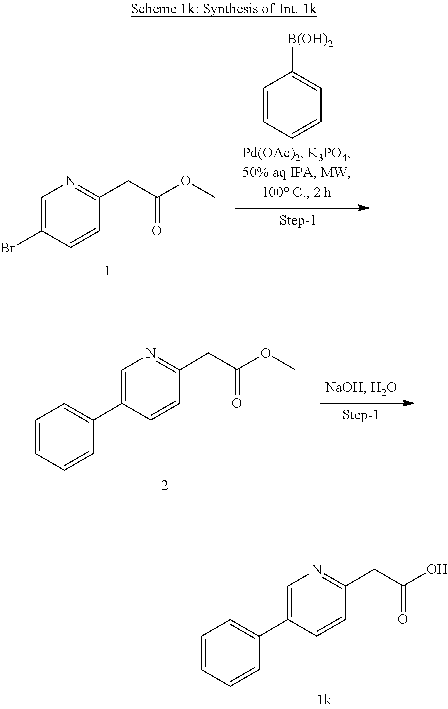 [SMILES: OB(O)c1ccccc1 ; COC(=O)Cc1ccc(Br)cn1 ; COC(=O)Cc1ccc(-c2ccccc2)cn1 ; O=C(O)Cc1ccc(-c2ccccc2)cn1]

Step-1 Synthesis of 2-(5-phenylpyridin-2-yl)acetic acid (2): A solution of methyl 2-(5-bromopyridin-2-yl)acetate (1, 250 mg, 1.09 mmol) and phenylboronic acid (132 mg, 1.09 mmol) in propan-2-ol and water (10 mL, 1:1) was degassed with argon for 10 min. After degassing, palladium acetate (24.4 mg, 0.109 mmol) and K₃PO₄ (1.25 g, 5.90 mmol) were added. The reaction vial was sealed with aluminium cap and irradiated in microwave at 100° C. for 1 h. After completion of the reaction (monitored by TLC), the reaction was filtered through a pad of celite and washed with IPA (50 mL). The combined organic layer was dried (Na₂SO₄) and concentrated in vacuo to give crude of the title compound (2, 265 mg crude) as a yellow solid as a black liquid which was used for next step without purification.

Step-2 Synthesis of 2-(5-phenylpyridin-2-yl)acetic acid (1k): To a stirred solution of methyl 2-(5-phenylpyridin-2-yl)acetate (2, 260 mg, 0.99 mmol) in MeOH (12 mL) was added H₂O (3 mL) followed by addition of sodium hydroxide (59 mg, 1.48 mmol). The reaction mixture was stirred at 70° C. for 1 h. After completion of reaction (monitored by TLC). The reaction mixture was diluted in water and the aqueous layer was extracted with ethyl acetate (2×30 mL). Aqueous layer was acidified with 5% aqueous NaHSO₄ solution (10 mL) and extracted with 10% MeOH in DCM (2×30 mL). The organic layers were combined, dried (Na₂SO₄) and concentrated in vacuo to give the title compound (1k, 250 mg crude) as a white solid which was used for further reaction.

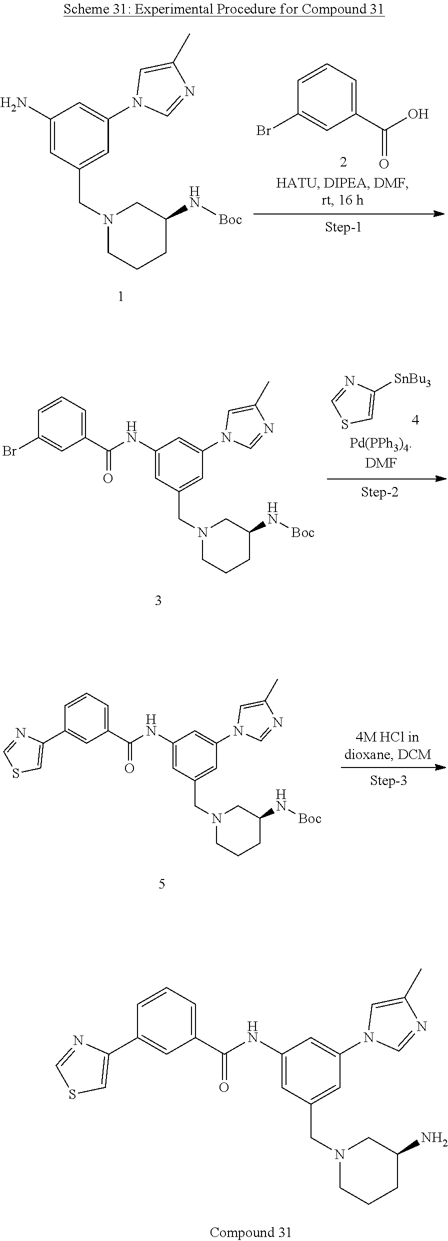

Step-1 Synthesis of tert-butyl N-(1-{[3-(3-bromobenzamido)-5-(4-methyl-1H-imidazol-1-yl)phenyl]methyl}piperidin-3-yl)carbamate (3): To a solution of amine 1 (575 mg, 1.49 mmol) and 3-bromo benzoic acid (2, 500 mg, 2.49 mmol) in DMF (10 mL), DIPEA (1.10 mL, 6.22 mmol) was added followed by addition of HATU (1.42 g, 3.73 mmol) at 0° C. and the reaction mixture was stirred the at room temperature for 16 h. Progress of the reaction was monitored by TLC, which showed consumption of starting material. After completion of reaction, the reaction mixture was diluted with water (50 mL) and extracted with EtOAc (2×50 ml), the resulting organic layers was dried over sodium sulfate and concentrated in vacuo. The residue was purified by flash column chromatography [normal phase, silica gel (100-200 mesh), gradient 0% to 5% MeOH in DCM] to give the title compound (3, 450 mg, 31%) as a brown solid. LCMS: m/z 569 (M+H)⁺ (ES+), 80%

Step-2 Synthesis of tert-butyl (S)-(1-(3-(4-methyl-1H-imidazol-1-yl)-5-(3-(thiazol-4-yl)benzamido)benzyl)piperidin-3-yl)carbamate (5): A mixture of 3 (100 mg, 0.26 mmol) and 4-(tributylstannyl)thiazole (4, 151 mg, 0.26 mmol) in DMF (3 mL) was degassed with N₂ for 10 minutes, Pd(PPh₃)₄ (0.169 g, 0.023 mmol) was added to it. The resulting reaction mixture was sealed with aluminium cap and stirred at 120° C. for 18 h. Progress of the reaction was monitored by TLC, which showed consumption of starting material. After completion of reaction, the reaction mixture was filtered through a pad of celite and washed with EtOAc (2×30 mL). The organic layers were combined, dried (Na₂SO₄) and concentrated in vacuo. The residue was purified by flash column chromatography [silica gel (100-200 mesh), gradient 10% to 20% EtOAc in Hexane] to enrich the purity up to 59% of the title compound (5, 120 mg) as a colourless gum. LCMS: m/z 573 (M+H)⁺ (ES+), 59%

Step-3 Synthesis of (S)—N-(3-((3-aminopiperidin-1-yl)methyl)-5-(4-methyl-1H-imidazol-1-yl)phenyl)-3-(thiazol-4-yl)benzamide (Compound 31): To a solution of 5 (0.12 g, 0.20 mmol) in DCM (2 mL), 4 M HCl in 1,4-dioxane (8.0 mL) was added and reaction mass was stirred at room temperature for 6 h. Progress of the reaction was monitored by TLC, which showed consumption of starting material. After completion of reaction, the resulting reaction mixture was concentrated in vacuo. The crude material was purified by prep-HPLC (reverse phase, T3 C18 (19×250) mm, 10 μm, gradient 40% to 60% ACN in water containing 0.1% TFA, RT: 10.28 min to give the title compound (Compound 31, 20 mg, 9.5%) as a yellow solid.

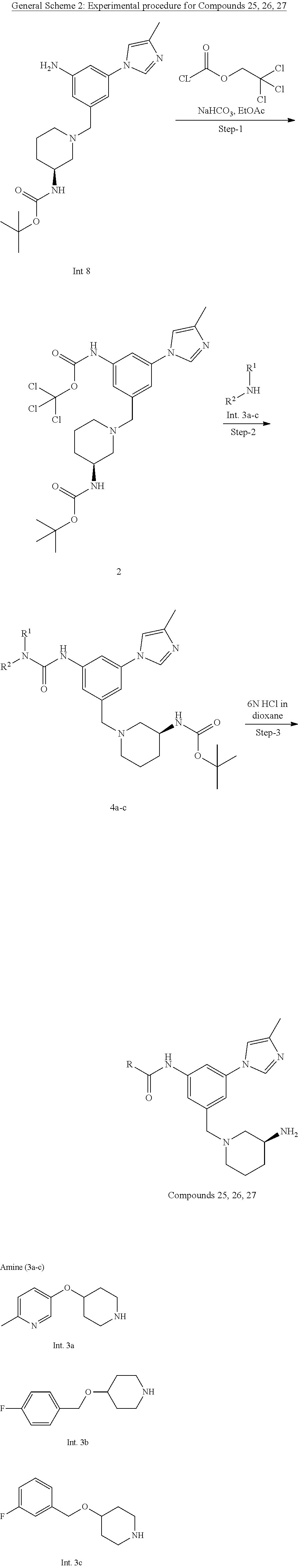

Structures of Compounds 25, 26 and 27:

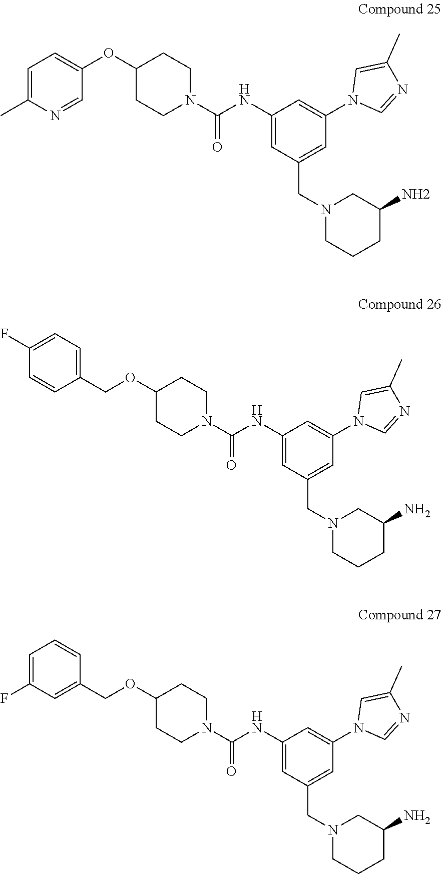 [SMILES: Cc1ccc(OC2CCN(C(=O)Nc3cc(CN4CCC[C@H](N)C4)cc(-n4cnc(C)c4)c3)CC2)cn1 ; Cc1cn(-c2cc(CN3CCC[C@H](N)C3)cc(NC(=O)N3CCC(OCc4ccc(F)cc4)CC3)c2)cn1 ; Cc1cn(-c2cc(CN3CCC[C@H](N)C3)cc(NC(=O)N3CCC(OCc4cccc(F)c4)CC3)c2)cn1]

Experimental Procedure for Intermediate 2:

Step-1 Synthesis of 2,2,2-trichloroethyl N-(3-{[(3R)-3-{[(tert-butoxy)carbonyl]amino}piperidin-1-yl]methyl}-5-(4-methyl-1H-imidazol-1-yl)phenyl)carbamate (2): To a solution amine Int. 8 (1.00 g, 2.59 mmol) in ethyl acetate (10.0 mL, 13.6 mmol), NaHCO₃ (436 mg, 5.19 mmol) was added followed by the addition of 2,2,2-trichloroethyl carbonochloridate (0.500 mL, 3.89 mmol) at 0° C. The reaction mixture was stirred at room temperature for 3 h. After completion of reaction (monitored by TLC), water was added in the reaction mixture and the aqueous layer was extracted with DCM (40 mL). The organic layer was dried (Na₂SO₄) and evaporated in vacuo to give the title compound (2, 1.20 g, 69%) as a brown solid. LCMS: m/z 561.20 (M+H)⁺ (ES+), 82%.

Experimental Procedure for Compound 25

Step-2 Synthesis of tert-butyl (S)-(1-(3-(4-methyl-1H-imidazol-1-yl)-5-(4-((6-methylpyridin-3-yl)oxy)piperidine-1-carboxamido)benzyl)piperidin-3-yl)carbamate (4a): To a solution of 2 (464 mg, 0.828 mmol) in dry DMF (3-5 mL/mmol), DIPEA (321 mg, 2.48 mmol) was added, followed by stirring of 10 min and the corresponding amine 3a (200 mg, 1.04 mmol) was added. The reaction mixture was stirred at 70° C. for 18 h. After completion of the reaction (monitored by TLC/LCMS), the reaction mixture was diluted with water (50 mL) and aqueous layer was extracted with ethyl acetate (2×50 mL). The organic layers were combined, dried (Na₂SO₄) and concentrated in vacuo to give crude material. The residue was purified by flash column chromatography [normal phase, silica gel (100-200 mesh), gradient 5% MeOH in DCM] to give the title compound (4a, 200 mg, 68%) as a brown solid. LCMS: m/z 604.20 (M+H)⁺ (ES+), 69%.

Step-3 (S)—N-(3-((3-aminopiperidin-1-yl)methyl)-5-(4-methyl-1H-imidazol-1-yl)phenyl)-4-((6-methylpyridin-3-yl)oxy)piperidine-1-carboxamide (Compound 25): To a solution of corresponding amide 4a (200 mg 0.32 mmol) in 1,4-dioxane (10 mL), 4M HCl in dioxane (10 mL) was added at 0° C. and the reaction mass was stirred at room temperature for 2 h. Progress of the reaction was monitored by TLC, which showed consumption of starting material. After completion of the reaction, the reaction mixture was concentrated in vacuo. The crude material was purified by prep-HPLC (reverse phase, X Bridge shield C18 (19×250) mm, 10 μm, gradient 47% to 53% ACN in water containing 5 mM Ammonium acetate, RT: 8.13 min to give the title compound (Compound 25, 23 mg, 21.21%) as a brown solid.

Experimental Procedure for Compound 26

Step 2 Synthesis of tert-butyl (S)-(1-(3-([1,1′-biphenyl]-3-carboxamido)-5-(4-methyl-1H-imidazol-1-yl)benzyl)piperidin-3-yl)carbamate (4b): To a solution of 2 (262 mg, 0.467 mmol) in dry DMF (3-5 mL/mmol), DIPEA (1.03 mL, 1.40 mmol) was added, followed by stirring of 10 min and the corresponding amine 3b (700 mg crude) was added. The reaction mixture was stirred at 70° C. for 18 h. After completion of the reaction (monitored by TLC/LCMS), the reaction mixture was diluted with water (50 mL) and aqueous layer was extracted with ethyl acetate (2×50 mL). The organic layers were combined, dried (Na₂SO₄) and concentrated in vacuo to give crude material. The residue was purified by flash column chromatography [normal phase, silica gel (100-200 mesh), gradient 5% MeOH in DCM] to give the title compound (4b, 200 mg) as a brown solid which was forwarded to the next step without analysis.

Step-3 Synthesis of tert-butyl (S)-(1-(3-(4-((4-fluorobenzyl)oxy)piperidine-1-carboxamido)-5-(4-methyl-1H-imidazol-1-yl)benzyl)piperidin-3-yl)carbamate (Compound 26): To a solution of corresponding amide 4b (200 mg 0.32 mmol) in 1,4-dioxane (10 mL), 4M HCl in dioxane (10 mL) was added at 0° C. and the reaction mass was stirred at room temperature for 2 h. Progress of the reaction was monitored by TLC, which showed consumption of starting material. After completion of the reaction, the reaction mixture was concentrated in vacuo. The crude material was purified by prep-HPLC (reverse phase, X Bridge shield C18 (19×250) mm, 10 μm, gradient 60% ACN in 70% 5 mM Ammonium acetate, RT: 12.29 min to give the title compound (Compound 26, 10 mg, 6%) as an off-white solid.

Experimental Procedure for Compound 27

Step-2 Synthesis of tert-butyl (S)-(1-(3-(4-((4-fluorobenzyl)oxy)piperidine-1-carboxamido)-5-(4-methyl-1H-imidazol-1-yl)benzyl)piperidin-3-yl)carbamate (4c): To a solution of 2 (401 mg, 0.71 mmol) in dry DMF (3-5 mL/mmol), DIPEA (368 mg, 2.36 mmol) was added, followed by stirring of 10 min and the corresponding amine 3c (300 mg, 1.43 mmol) was added. The reaction mixture was stirred at 70° C. for 18 h. After completion of the reaction (monitored by TLC/LCMS), the reaction mixture was diluted with water (50 mL) and aqueous layer was extracted with ethyl acetate (2×50 mL). The organic layers were combined, dried (Na₂SO₄) and concentrated in vacuo to give crude material. The residue was purified by flash column chromatography [normal phase, silica gel (100-200 mesh), gradient 5% MeOH in DCM] to give the title compound (4c, 200 mg) as a brown solid which was forwarded to the next step without analysis.

Step-3 Synthesis of (S)—N-(3-((3-aminopiperidin-1-yl)methyl)-5-(4-methyl-1H-imidazol-1-yl)phenyl)-4-((3-fluoro benzyl)oxy)piperidine-1-carboxamide (Compound 27): To a solution of corresponding amide 4c (190 mg 0.306 mmol) in DCM (3 mL), 4M HCl in dioxane (5 mL) was added at 0° C. and the reaction mass was stirred at room temperature for 2 h. Progress of the reaction was monitored by TLC, which showed consumption of starting material. After completion of the reaction, the reaction mixture was concentrated in vacuo. The crude material was purified by prep-HPLC (reverse phase, T3 C18 (19×250) mm, 10 μm, gradient 30% to 70% ACN in water containing 0.1% TFA, RT: 7.29 min to give the title compound (Compound 27, 31.0 mg, 19.45%) as an off-white solid.

Synthesis of Amine Coupling Partners Int. 3a, Int. 3b and Int. 3c

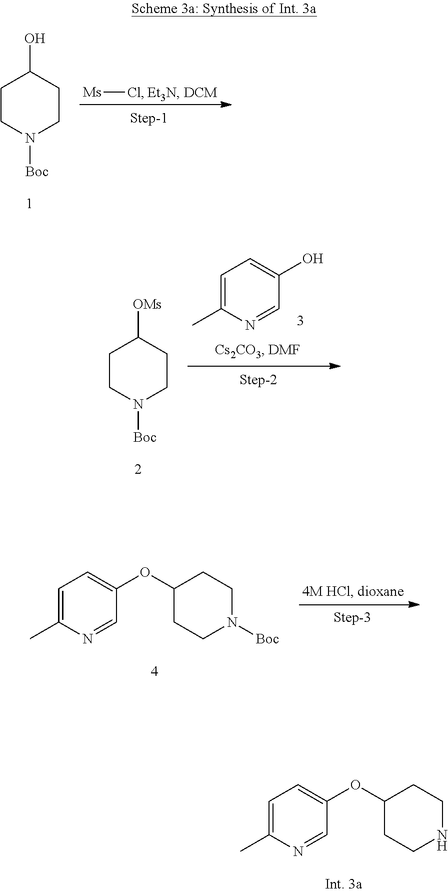

Step-1 Synthesis of tert-butyl 4-((methylsulfonyl)oxy)piperidine-1-carboxylate (2): To a stirred solution of 1 (4 g, 19.9 mmol) in DCM (25 mL), Et₃N (3.02 g, 29.8 mmol) was added and after 10 min, MsCl (2.73 g, 23.8 mmol) was added at 0° C. The reaction mixture was stirred at room temperature for 2 h. The reaction progress was monitored by TLC, which showed consumption of starting material. After completion of reaction, the reaction mixture was diluted with water (50 mL) and the aqueous layer was extracted with DCM (2×100 mL). The combined organic layers were dried (Na₂SO₄) and concentrated in vacuo to give the title compound (2, 5 g crude) as a white solid which was used for next step without purification.

Step-2 Synthesis of tert-butyl 4-((6-methylpyridin-3-yl)oxy)piperidine-1-carboxylate (4): To a stirred solution of 2 (2 g, 7.16 mmol) in DMF (10.0 mL), Cs₂CO₃ (2.32 g, 7.16 mmol) was added followed by addition of 6-methylpyridin-3-ol (3, 391 mg, 3.58 mmol) and the reaction mixture was stirred at 80° C. for 16 h. Progress of the reaction was monitored by TLC, which showed consumption of starting material. After completion of reaction, the reaction mixture was diluted with water (30 mL) and the aqueous layer was extracted with EtOAc (2×60 mL). The organic layers were combined, dried (Na₂SO₄) and concentrated in vacuo. The residue was purified by flash column chromatography [silica gel (100-200 mesh), gradient 0% to 2% EtOAc in Hexane] to give the title compound (4, 650 mg, 62.1%, over two steps) as a colourless liquid. LCMS: m/z 293.35 (M+H)⁺ (ES+), 93.66%

Step-3 Synthesis of 2-methyl-5-(piperidin-4-yloxy)pyridine hydrochloride (Int. 3a): To a stirred solution of tert-butyl 4-((6-methylpyridin-3-yl)oxy)piperidine-1-carboxylate (4, 650 mg, 2.22 mmol) in 1,4-dioxane (10.0 mL), hydrogen chloride in dioxane (7 mL, 4M) was added at 0° C. and stirred the reaction mixture at 50° C. for 6 h. After completion of the reaction (monitored by TLC), the reaction mixture was concentrated in vacuo to give the title compound (Int. 3a, 498 mg crude) as brown gum which was used for next step without purification. LCMS: m/z 193.08 (M+H)⁺ (ES+), 63.13%

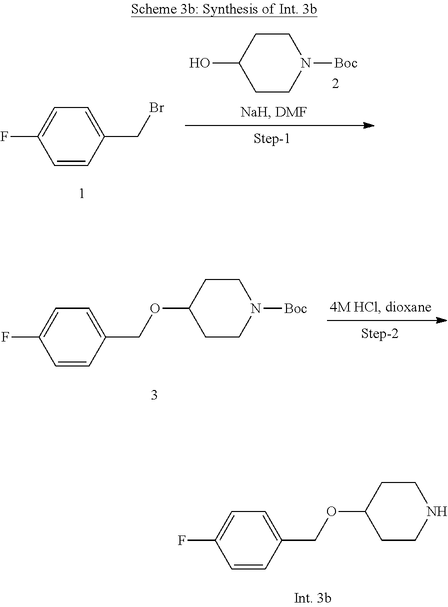 [SMILES: CC(C)(C)OC(=O)N1CCC(O)CC1 ; Fc1ccc(CBr)cc1 ; CC(C)(C)OC(=O)N1CCC(OCc2ccc(F)cc2)CC1 ; Fc1ccc(COC2CCNCC2)cc1]

Step-1 Synthesis of tert-butyl 4-((4-fluorobenzyl)oxy)piperidine-1-carboxylate (3): To a stirred solution of 2 (2.80 g, 14.9 mmol) in DMF (20.0 mL), NaH (1.18 g, 29.7 mmol) was added at 0° C. After 30 min stirring at same temperature, 1-(bromomethyl)-4-fluorobenzene (1, 2 g, 9.9 mmol) was added to it and the reaction mixture was stirred at 50° C. for 18 h. The progress of reaction was monitored by TLC. After completion, the reaction mixture was diluted with water and aqueous layer was extracted with EtOAc (2×100 mL). The organic layer was washed with brine (4×100 mL), dried (sodium sulfate) and concentrated in vacuo to give the residue which was purified by flash column chromatography [normal phase, silica gel (100-200 mesh), gradient 5% EtOAc in hexane] to give the title compound (3, 1.73 g) as colorless liquid which was forwarded to the next step without analysis.

Step-2 Synthesis of 4-[(4-fluorophenyl)methoxy]piperidine·HCl (Int. 3b): To a stirred solution of 3 (1.72 g, 12.9 mmol) in DCM (40.0 mL), HCl in dioxane (8.0 mL) was added at 0° C. and the reaction mixture was stirred at room temperature for 18 h. After completion of reaction (monitored by TLC), water was added to it and the aqueous was extracted with DCM (2×100 mL). The organic layer was washed with NaHCO₃ (4×100 mL), dried over sodium sulfate and concentrated in vacuo to give the title compound (Int. 3b, 931 mg, crude) as a brown solid.

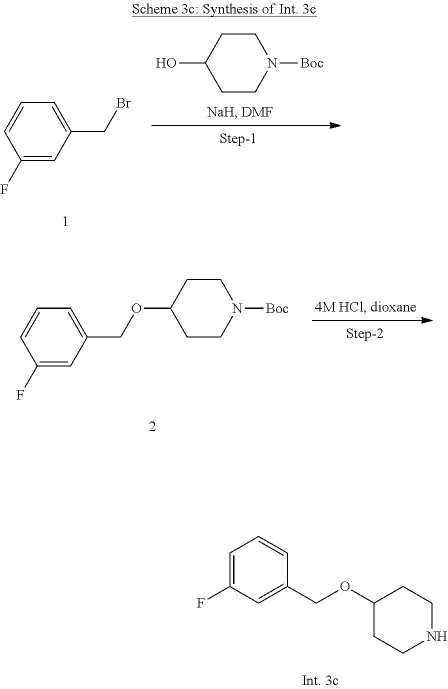 [SMILES: CC(C)(C)OC(=O)N1CCC(O)CC1 ; Fc1cccc(CBr)c1 ; CC(C)(C)OC(=O)N1CCC(OCc2cccc(F)c2)CC1 ; Fc1cccc(COC2CCNCC2)c1]

Step-1 Synthesis of tert-butyl 4-[(3-fluorophenyl)methoxy]piperidine-1-carboxylate (2): To a stirred solution of tert-butyl 4-hydroxypiperidine-1-carboxylate (3.00 g, 14.9 mmol) in DMF (20.0 mL) NaH (1.20 g, 3.4 eq., 50.0 mmol) was added at 0° C. After 30 min stirring at same temperature, 1-(bromomethyl)-3-fluorobenzene (1, 3.70 mL, 29.8 mmol) was added and the reaction mixture was stirred at 50° C. for 18 h. The progress of reaction was monitored by TLC. After completion, the reaction mixture was diluted with water and aqueous layer was extracted with EtOAc (2×100 mL). The organic layer was washed with brine (4×100 mL), dried (sodium sulfate) and concentrated in vacuo to give residue which was purified by flash column chromatography [normal phase, silica gel (100-200 mesh), gradient 5% EtOAc in hexane] to give the title compound (2, 4.00 g, 86%) as a yellowish liquid. LCMS: m/z 310.01 (M+H)+(ES+), 95%. ¹H-NMR (400 MHz; DMSO-d6): δ 1.49 (s, 9H), 1.80-1.88 (m, 3H), 3.00-3.10 (m, 3H), 3.50-3.71 (m, 3H), 4.53 (s, 2H), 7.05-7.20 (m, 3H), 7.35-7.42 (m, 1H)

Step-2 Synthesis of 4-[(3-fluorophenyl)methoxy]piperidine·HCl (Int. 3c): To a stirred solution of 2 (4.00 g, 12.9 mmol) in DCM (40.0 mL), 4M HCl in dioxane (15.0 mL) was added at 0° C. and the reaction mixture was stirred at room temperature for 18 h. After completion of reaction (monitored by TLC), water was added to it and the aqueous was extracted with DCM (2×100 mL). The organic layer was washed with NaHCO₃ (4×100 mL), dried (sodium sulfate) and concentrated in vacuo to give the title compound (Int. 3c, 1.50 g, 55%) as a brown solid. LCMS: m/z 210.01 (M+H)⁺ (ES+), 82%.

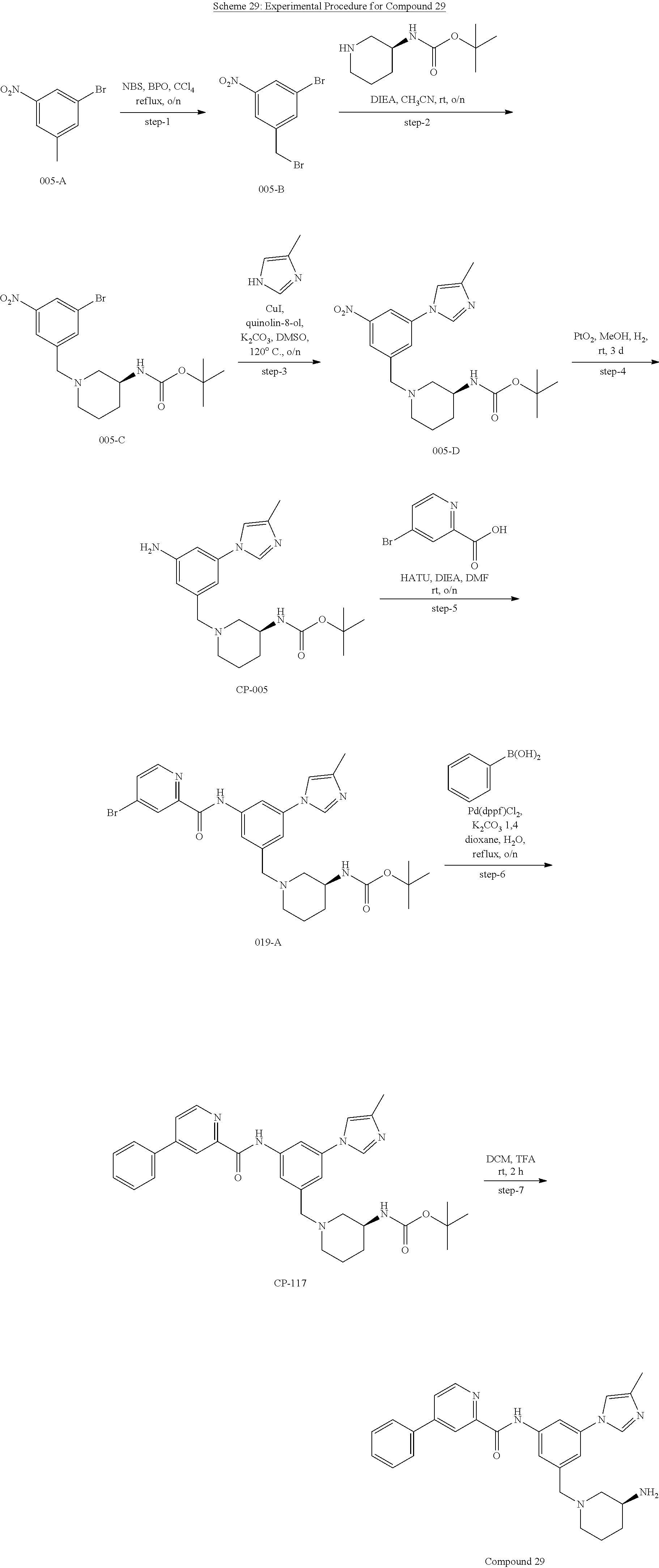

Step-1 The synthesis of 005-B: General Method A: to a mixture of 005-A (25.0 g, 115.7 mmol) and NBS (21.6 g, 121.5 mmol) in CCl₄ (250 mL) was added benzoyl peroxide (2.80 g, 11.6 mmol), and the reaction mixture was heated to reflux and stirred overnight. After cooling to room temperature, the precipitate was removed by filtration and the filtrate was evaporated under reduced pressure to give a residue. The residue was purified by column chromatography on silica gel (eluting with 5% EA in PE) to give 005-B (27.0 g, 80% yield) as a yellow solid.

Step-2 The synthesis of 005-C: General Method B1: to a mixture of 005-B (27.0 g, 92.2 mmol), (S)-tert-butyl piperidin-3-ylcarbamate (18.4 g, 92.2 mmol) in CH₃CN (300 mL) was added DIEA (23.8 g, 184.4 mmol), and the reaction mixture was stirred at room temperature overnight. After consumption of the starting material, water (300 mL) was added and the mixture was extracted with EtOAc (2×300 mL). The combined organic layers were washed with brine (300 mL), dried over anhydrous Na₂SO₄ and concentrated under reduced pressure to give a residue. The residue was purified by column chromatography on silica gel (eluting with 15% EA in PE) to give 005-C (25.0 g, 66% yield) as a yellow solid. ¹H NMR (400 MHz, CDCl₃) δ 8.19 (t, J=1.9 Hz, 1H), 8.04 (s, 1H), 7.74 (s, 1H), 4.82 (d, J=2.0 Hz, 1H), 3.71 (s, 1H), 3.51-3.38 (m, 2H), 2.54-2.52 (m, 1H), 2.30-2.24 (m, 3H), 1.63-1.50 (m, 3H), 1.37 (s, 9H).

Step-3 The synthesis of 005-D: General Method C: a suspension of 005-C (25.0 g, 60.5 mmol), 4-methyl-1H-imidazole (14.9 g, 181.5 mmol), K₂CO₃ (16.7 g, 121.0 mmol), CuI (3.5 g, 18.2 mmol) and quinolin-8-ol (1.8 g, 12.1 mmol) in DMSO (250 mL) was heated at 120° C. overnight under nitrogen atmosphere. After cooling to room temperature, water (300 mL) was added and the mixture was extracted with EtOAc (2×300 mL). The combined organic layers were washed with water (4×300 mL) and brine (300 mL) successively, dried over Na₂SO₄ and concentrated under reduced pressure to give a residue. The residue was purified by column chromatography on silica gel (eluting with 3% MeOH in DCM) to give 005-D (20.0 g, 99% yield) as a yellow solid. ¹H NMR (400 MHz, DMSO-d₆) δ 8.35 (s, 1H), 8.33 (s, 1H), 8.08 (s, 1H), 8.02 (s, 1H), 7.66 (s, 1H), 6.78 (d, J=8.0 Hz, 1H), 3.70-3.60 (m, 2H), 3.44 (s, 1H), 2.77 (d, J=7.7 Hz, 1H), 2.62 (s, 1H), 2.18 (s, 3H), 2.00-1.97 (m, 1H), 1.88-1.85 (m, 1H), 1.73-1.60 (m, 2H), 1.54-1.40 (m, 1H), 1.35 (s, 9H), 1.23-1.10 (m, 1H).

Step-4 The synthesis of CP-005: General Method D1: to a solution of 005-D (54.1 g, 130.2 mmol) in MeOH (1.0 L) was added PtO₂ (2.0 g), and the reaction mixture was stirred at room temperature for 3 days under hydrogen atmosphere. After consumption of the starting material, the reaction mixture was filtered through celite to remove the solid and the filtrate was evaporated under reduced pressure to give crude CP-005 (45.0 g, 90% yield) as a black solid. LC/MS (ESI): m/z=386.4 [M+1]⁺ RT=1.79 min ¹H NMR (400 MHz, DMSO-d₆) δ 7.91 (s, 1H), 7.24 (s, 1H), 6.73-6.71 (m, 1H), 6.57-6.55 (m, 2H), 6.48 (s, 1H), 5.35 (s, 2H), 3.38-3.29 (m, 3H), 2.77-2.74 (m, 1H), 2.65-2.63 (m, 1H), 2.14 (s, 3H), 1.86-1.81 (m, 1H), 1.74-1.68 (m, 2H), 1.62-1.59 (m, 1H), 1.44-1.41 (m, 1H), 1.35 (s, 9H), 1.15-1.07 (m, 1H).

Step-5 The synthesis of 019-A: General Method E: to a solution of CP-005 (45.0 g, 116.9 mmol) and 4-bromopicolinic acid (23.6 g, 116.9 mmol) in DMF (800 mL) was added HATU (53.3 g, 140.2 mmol) and DIEA (45.3 g, 350.6 mmol), and the reaction mixture was allowed to stir at room temperature overnight. After consumption of the starting material, the reaction mixture was diluted by water (800 mL) and extracted with EtOAc (2×800 mL). The organic extract was washed by water (4×1000 mL) and brine (1000 mL) successively, dried over anhydrous Na₂SO₄, filtered and evaporated under reduced pressure to give a residue. The residue was purified by column chromatography on silica gel (eluting with 3% MeOH in DCM) to give 019-A (45.0 g, 67% yield) as a yellow solid. LC/MS (ESI): m/z=568.8 [M+1]⁺ RT=2.24 min

Step-6 The synthesis of CP-117: General Method F: to a mixture of 019-A (40.0 g, 70.3 mmol), phenylboronic acid (12.9 g, 105.5 mmol), and K₂CO₃ (19.5 g, 140.6 mmol) in 1, 4-dioxane (500 mL) and H₂O (100 mL) was added Pd(dppf)Cl₂ (7.7 g, 10.5 mmol), and the reaction mixture was allowed to heat at reflux and stir overnight under argon atmosphere. After cooling to room temperature, it was diluted by water (500 mL) and extracted with EtOAc (2×400 mL). The organic extract was washed by brine (500 mL), dried over anhydrous Na₂SO₄ and evaporated under reduced pressure to give a residue. The residue was purified by column chromatography on silica gel (eluting with 3% MeOH in DCM) to give CP-117 (20.0 g, 50% yield) as a gray solid. LC/MS (ESI): m/z=567.0 [M+1]⁺ RT=6.81 min ¹H NMR (400 MHz, DMSO-d₆) δ 10.84 (s, 1H), 8.82 (d, J=4.8 Hz, 1H), 8.43 (d, J=1.2 Hz, 1H), 8.08-8.03 (m, 3H), 7.96-7.90 (m, 3H), 7.61-7.54 (m, 3H), 7.41 (s, 1H), 7.28 (s, 1H), 6.74 (d, J=7.6 Hz, 1H), 3.58-3.43 (m, 3H), 2.79 (d, J=7.6 Hz, 1H), 2.69-2.66 (m, 1H), 2.18 (s, 3H), 1.97-1.91 (m, 1H), 1.85-1.81 (m, 1H), 1.72-1.65 (m, 1H), 1.51-1.45 (m, 1H), 1.35 (s, 9H), 1.27-1.13 (m, 2H).

Step-7 The synthesis of Compound 29: General Method G: to a solution of CP-117 (20.0 g, 35.3 mmol) in DCM (150 mL) was added TFA (50 mL), and the reaction mixture was stirred at room temperature for 2 h. After consumption of the starting material, the volatiles were evaporated under reduced pressure to give a residue. The resulted residue was dissolved in DCM (300 mL) and basified by aqueous K₂CO₃ (300 mL). The organic layer was separated, washed by water (300 mL) and brine (300 mL) successively, dried over anhydrous Na₂SO₄, filtered and evaporated under reduced pressure to give Compound 29 (10.5 g, 64% yield) as a gray solid.

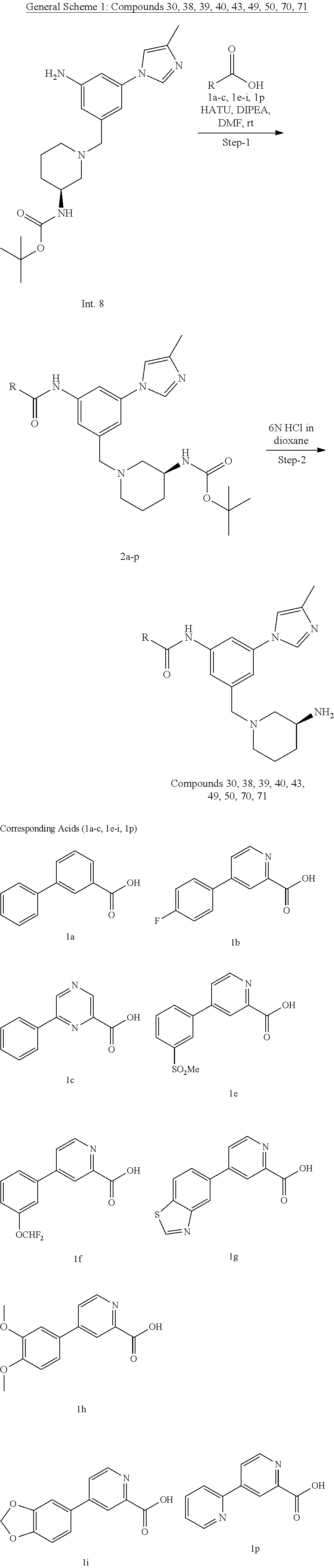

1a and 1c are available commercially.

Structures of Compounds 30, 38, 39, 40, 43, 49, 50, 70 and 71:

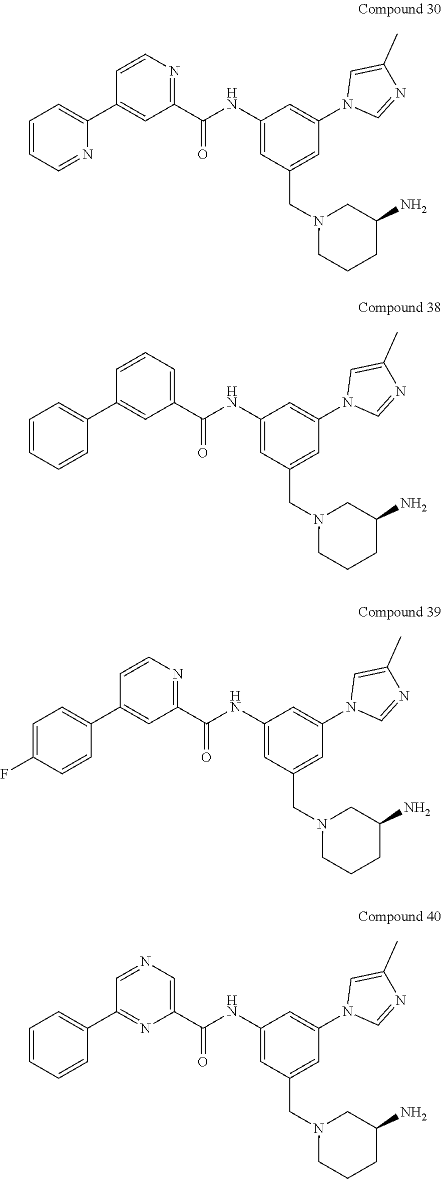 [SMILES: Cc1cn(-c2cc(CN3CCC[C@H](N)C3)cc(NC(=O)c3cc(-c4ccccn4)ccn3)c2)cn1 ; Cc1cn(-c2cc(CN3CCC[C@H](N)C3)cc(NC(=O)c3cccc(-c4ccccc4)c3)c2)cn1 ; Cc1cn(-c2cc(CN3CCC[C@H](N)C3)cc(NC(=O)c3cc(-c4ccc(F)cc4)ccn3)c2)cn1 ; Cc1cn(-c2cc(CN3CCC[C@H](N)C3)cc(NC(=O)c3cncc(-c4ccccc4)n3)c2)cn1]

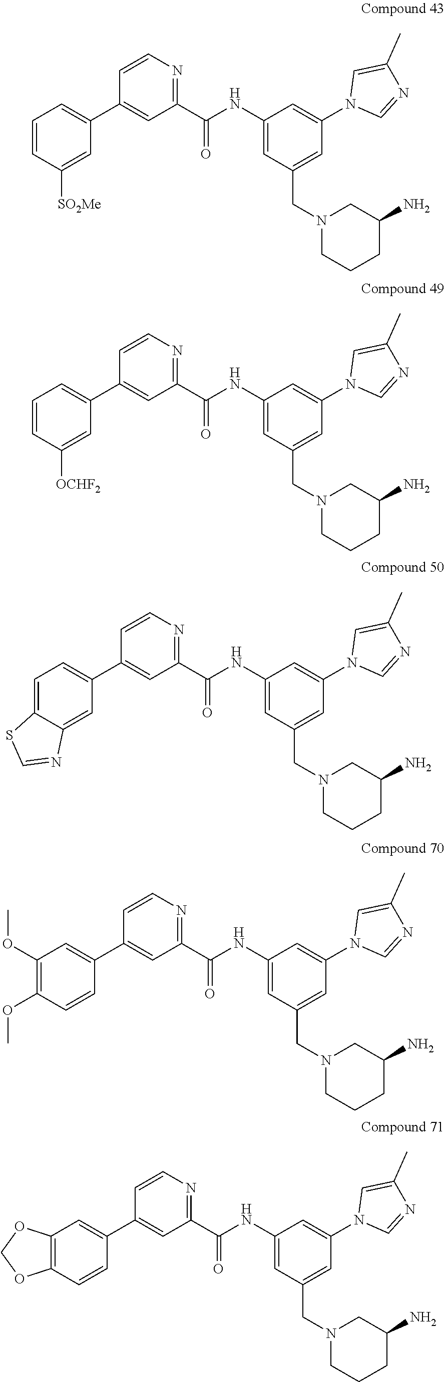 [SMILES: Cc1cn(-c2cc(CN3CCC[C@H](N)C3)cc(NC(=O)c3cc(-c4cccc(S(C)(=O)=O)c4)ccn3)c2)cn1 ; Cc1cn(-c2cc(CN3CCC[C@H](N)C3)cc(NC(=O)c3cc(-c4cccc(OC(F)F)c4)ccn3)c2)cn1 ; Cc1cn(-c2cc(CN3CCC[C@H](N)C3)cc(NC(=O)c3cc(-c4ccc5scnc5c4)ccn3)c2)cn1 ; COc1ccc(-c2ccnc(C(=O)Nc3cc(CN4CCC[C@H](N)C4)cc(-n4cnc(C)c4)c3)c2)cc1OC ; Cc1cn(-c2cc(CN3CCC[C@H](N)C3)cc(NC(=O)c3cc(-c4ccc5c(c4)OCO5)ccn3)c2)cn1]

Experimental Procedure for Compound 30

Step 1 Synthesis of tert-butyl N-[(3S)-1-[(3-{[2,4′-bipyridine]-2′-amido}-5-(4-methyl-1H-imidazol-1-yl)phenyl)methyl]piperidin-3-yl]carbamate (2p): To a solution of acid 1p (150 mg, 0.749 mmol) dry DMF (10 mL), DIPEA (0.33 mL, 1.87 mmol) and HATU (427 mg, 1.12 mmol) was added, followed by the stirring of 10 minutes and Int. 8 (173 mg, 0.450 mmol) was added at 0° C. The reaction mixture was stirred at room temperature for 16 h. After completion of reaction (monitored by TLC), the reaction mixture was diluted with water (50 mL) and extracted with EtOAc (2×50 mL), the combined organic layer was dried (Na₂SO₄) and concentrated in vacuo to give the title compound (2p, 100 mg crude) as brown semi-solid which was forwarded to the next step. MS (ESI+ve): m/z 568.60

Step 2 Synthesis of (S)—N-(3-((3-aminopiperidin-1-yl)methyl)-5-(4-methyl-1H-imidazol-1-yl)phenyl)-[2,4′-bipyridine]-2′-carboxamide (Compound 30): To a solution of amide 2p (150 mg) in 1,4-dioxane (10 mL), 4M HCl in dioxane (10 mL) was added at 0° C. and the reaction mass was stirred at room temperature for 2 h. Progress of the reaction was monitored by TLC, which showed consumption of starting material. After completion of the reaction, the reaction mixture was concentrated in vacuo. The crude material was concentrated in vacuo. The crude material was purified by prep-HPLC (reverse phase, X bridge C₈ (19×250) mm, 5 μm, gradient 25% to 75% ACN in water containing ammonium bi-carbonate, RT: 13.11 to give the title compound (Compound 30, 18 mg, 5% over 2 steps) as an off-white solid.

Experimental Procedure for Compound 38

Step 1 Synthesis of tert-butyl (S)-(1-(3-([1,1′-biphenyl]-3-carboxamido)-5-(4-methyl-1H-imidazol-1-yl)benzyl)piperidin-3-yl)carbamate (2a): To a solution of [1,1′-biphenyl]-3-carboxylic acid (1a, 150 mg, 0.76 mmol) dry DMF (3 mL), DIPEA (0.8 mL, 4.33 mmol) and HATU (494 mg, 1.93 mmol) was added, followed by the stirring of 10 minutes and Int. 8 (394 mg, 0.69 mmol) was added at 0° C. The reaction mixture was stirred at room temperature for 16 h. After completion of the reaction (monitored by TLC/LCMS) the mixture was diluted with water (50 mL) and extracted with EtOAc (2×50 mL), the combined organic layer was dried over sodium sulfate and concentrated in vacuo. The residue was purified by flash column chromatography [normal phase, silica gel (100-200 mesh), gradient 30% to 40% EtOAc in Hexane] to give the title compound (2a, 394 mg, 92%) as an off-white semi-solid. LCMS: m/z 566.15 (M+H)⁺ (ES+), 84.24%

Step 2 Synthesis of (S)—N-(3-((3-aminopiperidin-1-yl)methyl)-5-(4-methyl-1H-imidazol-1-yl)phenyl)-[1,1′-biphenyl]-3-carboxamide·HCl (Compound 38): To a solution of amide 2a (100 mg) in DCM (10 mL), 4M HCl in dioxane (10 mL) was added at 0° C. and the reaction mass was stirred at room temperature for 2 h. Progress of the reaction was monitored by TLC, which showed consumption of starting material. After completion of the reaction, the reaction mixture was concentrated in vacuo. The residue was purified by trituration with ACN/diethyl ether to give the title compound as the HCl salt (Compound 38, 79 mg, 96%) as an off-white solid.

Experimental Procedure for Compound 39

Step 1 Synthesis of tert-butyl N-[(3S)-1-({3-[4-(4-fluorophenyl)pyridine-2-amido]-5-(4-methyl-1H-imidazol-1-yl)phenyl}methyl)piperidin-3-yl]carbamate (2b): To a solution of acid 1b (269 mg, 1.24 mmol) dry DMF (10 mL), DIPEA (0.548 mL, 3.09 mmol) and HATU (706 mg, 1.86 mmol) was added, followed by the stirring of 10 minutes and Int. 8 (286 mg, 0.743 mmol) was added at 0° C. The reaction mixture was stirred at room temperature for 16 h. After completion of the reaction (monitored by TLC/LCMS) the mixture was diluted with water (50 mL) and extracted with EtOAc (2×50 mL), the combined organic layer was dried over sodium sulfate and concentrated in vacuo. The crude was purified by flash column chromatography [normal phase, silica gel (100-200 mesh), gradient 30% to 40% EtOAc in Hexane] to give the title compound (2b, 320 mg, 44%) as a brown solid. LCMS: m/z 585.29 (M+H)⁺ (ES+), 93%

Step 2 Synthesis of N-(3-{[(3S)-3-aminopiperidin-1-yl]methyl}-5-(4-methyl-1H-imidazol-1-yl)phenyl)-4-(4-fluorophenyl)pyridine-2-carboxamide (Compound 39): To a solution of amide 2a (200 mg) in DCM (15 mL), 4M HCl in dioxane (15 mL) was added at 0° C. and the reaction mass was stirred at room temperature for 2 h. Progress of the reaction was monitored by TLC, which showed consumption of starting material. After completion of the reaction, the reaction mixture was concentrated in vacuo. The residue was purified by prep-HPLC (reverse phase, X Bridge shield C18 (19×250) mm, 10 μm, gradient 20% to 80% ACN in water containing 5 mM Ammonium acetate, RT: 11.45 min (Compound 39, 24.0 mg, 14.5%) as an off-white solid.

Experimental Procedure for Compound 40

Step 1 Synthesis of tert-butyl N-[(3S)-1-{[3-(4-methyl-1H-imidazol-1-yl)-5-(6-phenylpyrazine-2-amido)phenyl]methyl}piperidin-3-yl]carbamate (2c): To a solution of acid 1c (200 mg, 0.999 mmol) dry DMF (15 mL), DIPEA (0.442 mL, 2.50 mmol) and HATU (HATU (353 mg, 1.50 mmol) was added, followed by the stirring of 10 minutes and Int. 8 (231 mg, 599 μmol) was added at 0° C. The reaction mixture was stirred at room temperature for 16 h. After completion of reaction (monitored by TLC/LCMS), the reaction mixture was diluted with water (50 mL) and extracted with EtOAc (2×50 ml), the combined organic layers was dried over sodium sulfate and concentrated in vacuo to give the title compound (2c, 200 mg crude) as a brown solid. LCMS: m/z 568.3 (M+H)⁺ (ES+), 75%

Step 2 Synthesis of N-(3-{[(3S)-3-aminopiperidin-1-yl]methyl}-5-(4-methyl-1H-imidazol-1-yl)phenyl)-6-phenylpyrazine-2-carboxamide (Compound 40): To a solution of amide 2c (194 mg) in DCM (15 mL), 4M HCl in dioxane (15 mL) was added at 0° C. and the reaction mass was stirred at room temperature for 2 h. Progress of the reaction was monitored by TLC, which showed consumption of starting material. After completion of the reaction, the reaction mixture was concentrated in vacuo. The crude material was purified by prep-HPLC (reverse phase, X Select Fluoro phenyl C18 (19×250) mm, 5 μm, gradient 20% to 80% ACN in water containing 5 mM Ammonium acetate, RT: 11.25 to give the title compound (Compound 40, 60.0 mg, 37.5%) as an off-white solid.

Experimental Procedure for Compound 43

Step 1 Synthesis of tert-butyl (S)-(1-(3-(4-methyl-1H-imidazol-1-yl)-5-(3′-(methylsulfonyl)-[1,1′-biphenyl]-3-carboxamido)benzyl)piperidin-3-yl)carbamate (2e): To a solution of acid 1e (0.32 g, 1.16 mmol) dry DMF (10 mL), DIPEA (0.0.45 g, 3.5 mmol) and HATU (0.67 g, 1.7 mmol) was added, followed by the stirring of 10 minutes and Int. 8 (0.45 g, 1.16 mmol) was added at 0° C. The reaction mixture was stirred at room temperature for 16 h. After completion of reaction, the reaction mixture was diluted with water (50 mL) and extracted with EtOAc (2×50 mL), the combined organic layers was dried (Na₂SO₄) and concentrated in vacuo. The residue was purified by flash column chromatography [normal phase, silica gel (100-200 mesh), gradient 0% to 5% MeOH in DCM] to give the title compound (2e, 0.25 g) as a white solid which was used for next step without analysis.

Step-2 Synthesis of (S)—N-(3-((3-aminopiperidin-1-yl)methyl)-5-(4-methyl-1H-imidazol-1-yl)phenyl)-4-(3-(methylsulfonyl)phenyl)picolinamide (Compound 43): To a solution of amide 2e (210 mg) in 1,4-dioxane (2 mL) and DCM (5 mL), 4M HCl in dioxane (2 mL) was added at 0° C. and the reaction mass was stirred at room temperature for 2 h. Progress of the reaction was monitored by TLC, which showed consumption of starting material. After completion of the reaction, the reaction mixture was concentrated in vacuo. The crude material was purified by prep-HPLC (reverse phase, X Bridge shield C18 (19×250) mm, 10 μm, gradient 30% to 70% ACN in water containing 5 mM Ammonium acetate, RT: 9.10 to give the title compound (Compound 43, 62 mg, 9.8% over 2 steps, ˜14% acetate w/w present) as a white solid.

Experimental Procedure for Compound 49

Step 1 Synthesis of tert-butyl N-{3-[(3-{4-[3-(difluoromethoxy)phenyl]pyridine-2-amido}-5-(4-methyl-1H-imidazol-1-yl)phenyl)methyl]cyclohexyl}carbamate (2f): To a solution of acid 1f (130 mg, 0.490 mmol) dry DMF (5 mL), DIPEA (0.341 mL, 1.96 mmol) and HATU (150 mg, 0.637 mmol) was added, followed by the stirring of 10 minutes and Int. 8 (100 mg, 0.43 mmol) was added at 0° C. The reaction mixture was stirred at room temperature for 16 h. After completion of reaction, the reaction mixture was diluted with water (50 mL) and extracted with EtOAc (2×50 ml), the resulting organic layers was dried (Na₂SO₄) and concentrated in vacuo to give the title compound (2f, 600 mg crude) which was forwarded to next step without analysis.

Step 2 Synthesis of N-(3-{[(3S)-3-aminopiperidin-1-yl]methyl}-5-(4-methyl-1H-imidazol-1-yl)phenyl)-4-[3-(difluoromethoxy)phenyl]pyridine-2-carboxamide (Compound 49): To a solution of amide 2f (600 mg, crude) in 1,4-dioxane (5 mL), 4M HCl in dioxane (1 mL) was added at 0° C. and the reaction mass was stirred at room temperature for 2 h. Progress of the reaction was monitored by TLC, which showed consumption of starting material. After completion of the reaction, the reaction mixture was concentrated in vacuo. The crude material was purified by prep-HPLC (reverse phase, Sunfire C18 (19×250) mm, 10 μm, gradient 20% to 80% ACN in water containing 0.1% TFA, RT: 12.35 to give the title compound (Compound 49, 29.0 mg, 11% over 2 steps).

Experimental Procedure for Compound 50

Step 1 Synthesis of tert-butyl N-[(3S)-1-({3-[4-(1,3-benzothiazol-5-yl)pyridine-2-amido]-5-(4-methyl-1H-imidazol-1-yl)phenyl}methyl)piperidin-3-yl]carbamate (2g): To a solution of acid 1g (250 mg, 0.975 mmol) dry DMF (5 mL), DIPEA (1.08 mL, 5.85 mmol) and HATU (0.67 g, 1.7 mmol) was added, followed by the stirring of 10 minutes and Int. 8 (0.376 g, 0.975 mmol) was added at 0° C. The reaction mixture was stirred at room temperature for 16 h. After completion, water (20 mL) was added to the reaction mixture and the aqueous layer was extracted with ethyl acetate (2×30 mL). The organic layers were combined, dried (Na₂SO₄) and concentrated in vacuo. The residue was purified by flash column chromatography [normal phase, silica gel (100-200 mesh), gradient 0% to 5% MeOH in DCM] to give to give the title compound (2g, 365 mg, 53.99%) as a yellow solid. LCMS: m/z 625.66, (M+H)⁺ (ES+), 90.11%

Step 2 Synthesis of N-(3-{[(3S)-3-aminopiperidin-1-yl]methyl}-5-(4-methyl-1H-imidazol-1-yl)phenyl)-4-(1,3-benzothiazol-5-yl)pyridine-2-carboxamide (Compound 50): To a solution of amide 2g (365 mg) in DCM (15 mL), 4M HCl in dioxane (10 mL) was added at 0° C. and the reaction mass was stirred at room temperature for 2 h. Progress of the reaction was monitored by TLC, which showed consumption of starting material. After completion of the reaction, the reaction mixture was concentrated in vacuo. The crude material was purified by prep-HPLC (reverse phase, Xbridge C-8 (19×250) mm, 5 μm, gradient 25% to 47% ACN in water containing 0.1% Ammonium Hydroxide in Water, RT: 14.6 min to give the title compound (Compound 50, 18.0 mg, 5.87%) as a white solid.

Experimental Procedure for Compound 70

Step 1 Synthesis of tert-butyl (S)-(1-(3-(4-(3,4-dimethoxyphenyl)picolinamido)-5-(4-methyl-1H-imidazol-1-yl)benzyl)piperidin-3-yl)carbamate (2h): To a solution of acid 1h (200 mg crude, 0.77 mmol) dry DMF (5 mL), DIPEA (0.42 mL, 2.31 mmol) and HATU (381 mg, 1.00 mmol) was added, followed by the stirring of 10 minutes and Int. 8 (297 mg, 0.77 mmol) was added at 0° C. The reaction mixture was stirred at room temperature for 16 h. After completion, water (20 mL) was added to the reaction mixture and the aqueous layer was extracted with ethyl acetate (2×30 mL). The organic layers were combined, dried (Na₂SO₄) and concentrated in vacuo. The residue was enriched up to 36.5% purity by flash column chromatography [Silica gel (100-200 mesh), gradient 0% to 5% MeOH in DCM] to give the title compound (2h, 200 mg) as a yellow semi-solid. LCMS: m/z 627.57 (M+H)⁺ (ES+), 36.58%

Step 2 Synthesis of (S)—N-(3-((3-aminopiperidin-1-yl)methyl)-5-(4-methyl-1H-imidazol-1-yl)phenyl)-4-(3,4-dimethoxyphenyl)picolinamide (Compound 70): To a solution of amide 2h (200 mg) in DCM (10 mL), 4M HCl in dioxane (10 mL) was added at 0° C. and the reaction mass was stirred at room temperature for 2 h. Progress of the reaction was monitored by TLC, which showed consumption of starting material. After completion of the reaction, the reaction mixture was concentrated in vacuo. The residue was purified by prep-HPLC (reverse phase, X Bridge C18 (19×250) mm, 10 μm, gradient 30% to 70% ACN in water containing 5 mM Ammonium acetate, RT: 11.24 min to give the title compound (Compound 70, 7.0 mg, 2%) as a white solid.

Experimental Procedure for Compound 71

Step 1 Synthesis of tert-butyl (S)-(1-(3-(4-(benzo[d][1,3]dioxol-5-yl)picolinamido)-5-(4-methyl-1H-imidazol-1-yl)benzyl)piperidin-3-yl)carbamate (2i): To a solution of acid 1i (180 mg, 0.30 mmol) dry DMF (4 mL), DIPEA (0.3 mL, 1.64 mmol) and HATU (313 mg, 0.82 mmol) was added, followed by the stirring of 10 minutes and Int. 8 (159 mg, 0.41 mmol) was added at 0° C. The reaction mixture was stirred at room temperature for 16 h. After completion, water (20 mL) was added to the reaction mixture and the aqueous layer was extracted with ethyl acetate (2×30 mL). The organic layers were combined, dried (Na₂SO₄) and concentrated in vacuo. The residue was enriched up to 53.3% purity through flash column chromatography [normal phase, silica gel (100-200 mesh), gradient 0% to 5% MeOH in DCM] to give the title compound (2i, 152 mg) as a brown semi-solid. LCMS: m/z 611.55 (M+H)⁺ (ES+), 53.3%

Step 2 Synthesis of (S)—N-(3-((3-aminopiperidin-1-yl)methyl)-5-(4-methyl-1H-imidazol-1-yl)phenyl)-4-(benzo[d][1,3]dioxol-5-yl)picolinamide. TFA (Compound 71): To a solution of amide 2i (150 mg) in DCM (10 mL), 4M HCl in dioxane (10 mL) was added at 0° C. and the reaction mass was stirred at room temperature for 2 h. Progress of the reaction was monitored by TLC, which showed consumption of starting material. After completion of the reaction, the reaction mixture was concentrated in vacuo. The residue was purified by prep-HPLC (reverse phase, X Bridge Shield C18 (19×250) mm, 10μ, gradient 33% to 65% ACN in water containing 5 mM Ammonium acetate, RT: 12.90 min to give the title compound as the TFA salt (Compound 71, 19 mg, 5%, over two step) as an off-white semi-solid.

Synthesis of Corresponding Acids: 1b, 1e, 1f, 1g, 1h, 1i and 1p:

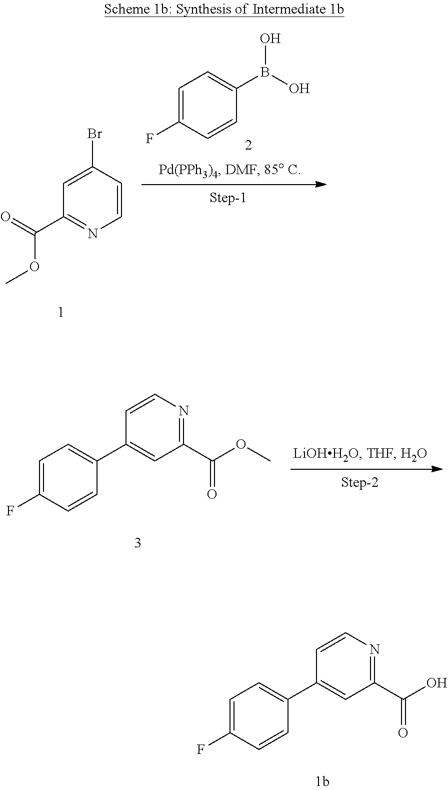 [SMILES: COC(=O)c1cc(Br)ccn1 ; OB(O)c1ccc(F)cc1 ; COC(=O)c1cc(-c2ccc(F)cc2)ccn1 ; O=C(O)c1cc(-c2ccc(F)cc2)ccn1]

Step-1 Synthesis of methyl 4-(4-fluorophenyl)pyridine-2-carboxylate (3): A stirred solution of methyl 4-bromopicolinate (1.50 g, 6.94 mmol), (4-fluorophenyl)boronic acid (1.00 g, 7.15 mmol) and K₂CO₃ (2.96 g, 21.4 mmol) in DMF (10.0 mL) was degassed with N₂ for 10 min and Pd(PPh₃)₄ (323 mg, 0.357 mol) was added to it. The resulting mixture was stirred at 150° C. for 18 h. The progress of reaction monitored by TLC, which showed consumption of starting material. After completion, water was added and extracted with EtOAc (2×100 mL). The organic layer was washed with cold brine solution (4×100 mL) and dried (sodium sulfate) and a concentrated in vacuo to give the title compound (3, 230 mg, crude) as brown solid. The obtained crude was used in the next step without analysis.

Step-2 Synthesis of 4-(4-fluorophenyl)pyridine-2-carboxylic acid (1b): To a stirred solution of methyl 4-(4-fluorophenyl)pyridine-2-carboxylate (224 mg crude) in THF (20.0 mL) and H₂O (3 mL), LiOH·H₂O (407 mg, 9.69 mmol) was added. The reaction mixture was stirred at 60° C. for 18 h. Reaction progress was monitored by TLC which showed consumption of starting material. The reaction mixture was diluted in water (20 mL) and extracted with ethyl acetate (3×30 mL). The aqueous layer was acidified with 6N HCl and extracted with 10% MeOH in DCM. The combined organic layer was washed with cold brine solution and dried (Na₂SO₄). The solvent was evaporated in vacuo to give the title compound (1b, 275 mg) as a white solid. LCMS: m/z 218.25 (M+H)⁺ (ES+), 92%.

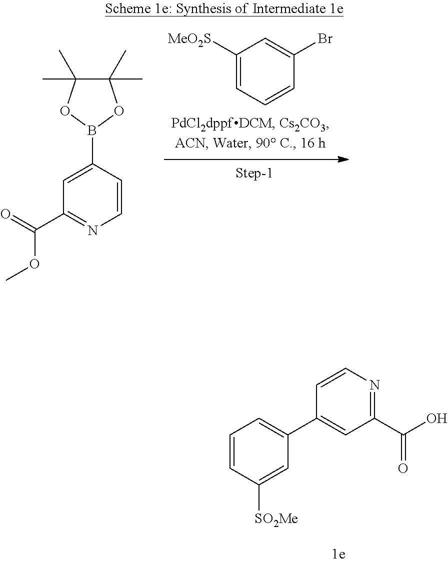 [SMILES: COC(=O)c1cc(B2OC(C)(C)C(C)(C)O2)ccn1 ; CS(=O)(=O)c1cccc(Br)c1 ; CS(=O)(=O)c1cccc(-c2ccnc(C(=O)O)c2)c1]

Step-1 Synthesis of 4-(3-(methylsulfonyl)phenyl)picolinic acid (1e): To a stirred solution of methyl 4-(4,4,5,5-tetramethyl-1,3,2-dioxaborolan-2-yl)picolinate (0.7 g, 3.2 mmol) and 1-bromo-3-(methylsulfonyl)benzene (1.37 g, 4.8 mmol) in ACN (15 mL, degassed with N₂ for 10 min), cesium carbonate (3.14 g, 9.7 mmol) and PdCl₂(dppf) (0.116 g, 0.016 mmol) were added to reaction mixture and heated to 90° C. for 16 h. Progress of the reaction was monitored by TLC or LCMS. After consumption of starting material, the reaction mixture was diluted with water (50 mL) and extracted with ethyl acetate (2×50 mL). The aqueous layer was acidified with 6N HCl (10 mL), brown precipitate was appeared. This was filtered in sintered funnel and washed with water (50 mL) and dried in vacuo to give the title compound as light-brown solid (1e, 0.38 g crude). The obtained crude material was used in the next step without analysis.

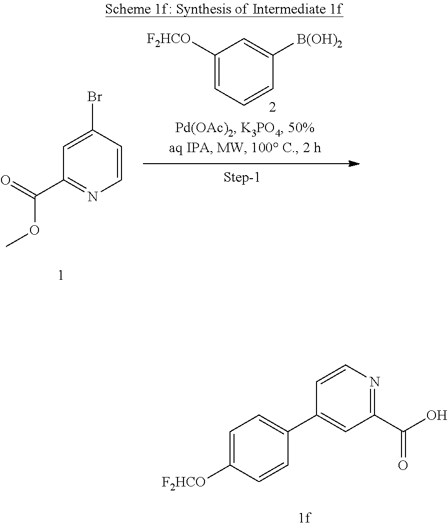 [SMILES: COC(=O)c1cc(Br)ccn1 ; OB(O)c1cccc(OC(F)F)c1 ; O=C(O)c1cc(-c2ccc(OC(F)F)cc2)ccn1]

Step-1 Synthesis of 4-(4-(difluoromethoxy)phenyl)picolinic acid (1f): A solution of methyl 4-bromopicolinate (1, 115 mg, 0.53 mmol) and (3-(difluoromethoxy)phenyl)boronic acid (2, 100 mg, 0.53 mmol) in propan-2-ol and water (10 mL, 1:1) was degassed with argon for 10 min. After degassing, palladium acetate (12 mg, 0.05 mmol) and K₃PO₄ (339 mg, 1.60 mmol) were added. The reaction vial was sealed with aluminium cap and irradiated in microwave at 100° C. for 2 h. After completion of the reaction (monitored by TLC), the reaction was filtered through a pad of celite and washed with IPA (50 mL). Organic layers were combined, dried (Na₂SO₄) and concentrated in vacuo to give crude of the title compound (1f, 150 mg crude) as a black liquid. This crude material was used in the next step without analysis.

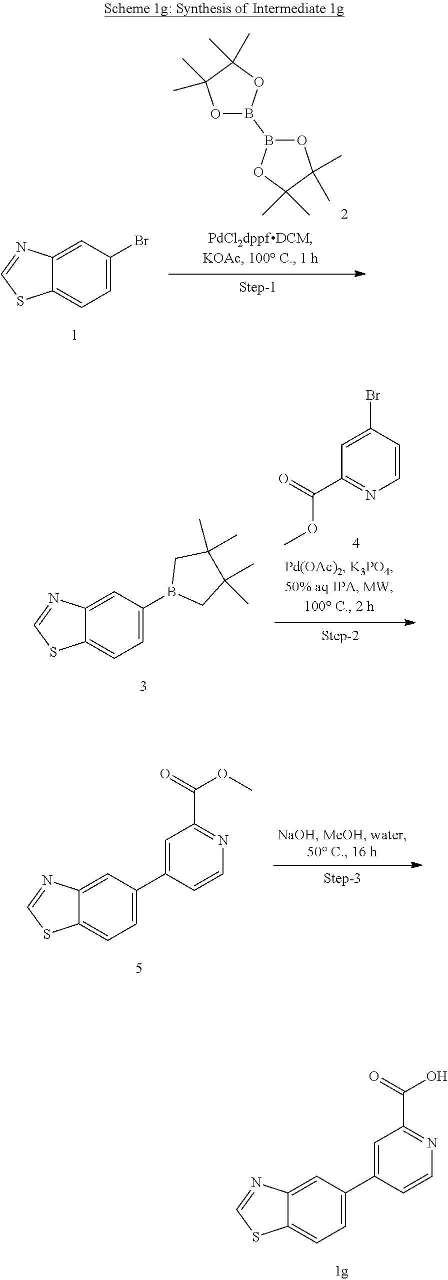

Step-1 Synthesis of 5-(4,4,5,5-tetramethyl-1,3,2-dioxaborolan-2-yl)benzo[d]thiazole (3): A solution of 5-bromobenzo[d]thiazole (1, 500 mg, 2.34 mmol) and bispinacolatodiborane (2, 593 mg, 2.34 mmol) in dioxane (12 mL) was degassed with argon for 10 min. KOAc (688 mg, 7.01 mmol) and PdCl₂dppf·DCM (95 mg, 0.12 mmol) were added to the reaction mixture and the reaction vial was sealed with aluminium cap. The reaction mixture was stirred at 100° C. for 1 h. After completion (monitored by TLC), reaction was filtered through a pad of celite and washed with EtOAc (2×50 mL). The filtrate was evaporated to dryness under reduced pressure to give the title compound (3, 982 mg crude) as a dark brown solid which was used in the next step without analysis.

Step-2 Synthesis of methyl 4-(benzo[d]thiazol-5-yl)picolinate (5): A solution of methyl 4-bromopicolinate (4, 300 mg, 1.39 mmol) and 5-(4,4,5,5-tetramethyl-1,3,2-dioxaborolan-2-yl)benzo[d]thiazole (3, 544 mg crude) in propan-2-ol and water (30 mL, 1:1) was degassed with argon for 10 min. After degassing, palladium diacetate (94 mg, 0.42 mmol) and K₃PO₄ (884 mg, 4.17 mmol) were added. The reaction vial was sealed with aluminium cap and irradiated in microwave at 100° C. for 2 h. After completion of the reaction (monitored by TLC), the reaction was filtered through a pad of celite and washed with IPA (50 mL). Organic layers were combined, dried (Na₂SO₄) and concentrated in vacuo to give crude of the title compound (3, 525 mg crude) as a black liquid which was used for next step without analysis.

Step-3 Synthesis of 4-(benzo[d]thiazol-5-yl)picolinic acid (1g): To a stirred solution of methyl 4-(benzo[d]thiazol-5-yl)picolinate (3, 522 mg crude) in THF (15.0 mL) and water (3.00 mL), sodium hydroxide (232 mg, 5.79 mmol) was added. The reaction mixture was stirred at 60° C. for 3 h. After completion of the reaction (monitored by TLC), reaction mixture was concentrated and water (20 mL) was added. Aqueous layer was extracted with ether (2×20 mL) and organic layer was separated. Aqueous layer was acidified with aq NaHSO₄ solution (10 mL). The aqueous layer was extracted with 10% MeOH in DCM (2×30 mL). The organic layers were combined, dried (Na₂SO₄) and concentrated in vacuo to give the title compound (1g, 432 mg crude) as a brown gummy which was used for next step without analysis.

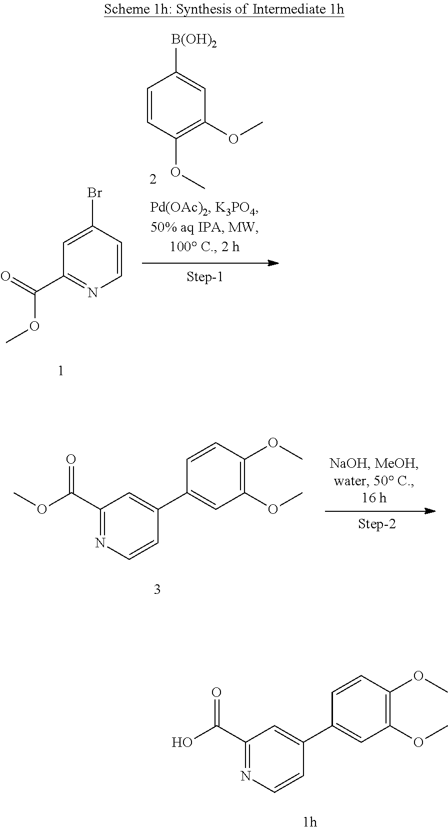

Step-1 Synthesis of methyl 4-(3,4-dimethoxyphenyl)picolinate (3): A solution of methyl 4-bromopyridine-2-carboxylate (1, 500 mg, 2.31 mmol) and (3,4-dimethoxyphenyl)boronic acid (2, 421 mg, 2.31 mmol) in propan-2-ol and water (80 mL, 1:1) was degassed with argon for 10 min. After degassing, palladium diacetate (208 mg, 0.93 mmol) and K₃PO₄ (1.47 g, 6.94 mmol) were added. The reaction vial was sealed with aluminium cap and irradiated in microwave at 100° C. for 2 h. After completion of the reaction, the reaction was filtered through a pad of celite and washed with IPA (50 mL). Organic layers were combined, dried (Na₂SO₄) and concentrated in vacuo to give crude mixture of the title compound (3, 400 mg crude) as a yellowish gum which was used for next step without analysis.

Step-2 Synthesis of 4-(3,4-dimethoxyphenyl)picolinic acid (1h): Crude mixture of methyl 4-(3,4-dimethoxyphenyl)pyridine-2-carboxylate and 4-(3,4-dimethoxyphenyl)picolinic acid (3 and 4, 400 mg crude, 1.46 mmol) was stirred in methanol (10.0 mL) and sodium hydroxide (293 mg, 7.32 mmol) in water (2.00 mL) was added to it. The reaction mixture was then heated at 50° C. for 16 h. The reaction progress was monitored by TLC, which showed consumption of starting material. After completion of the reaction, reaction mixture was concentrated under reduced pressure and water (20 mL) was added. Aqueous layer was extracted with ether (2×20 mL) and organic layer was separated. Aqueous layer was acidified with aq NaHSO₄ solution (pH=4-5). The aqueous layer was extracted with ethyl acetate (2×30 mL). The organic layers were combined, dried (Na₂SO₄) and concentrated in vacuo to give the title compound (1h, 200 mg crude) as brownish solid. LCMS: m/z 259.91 (M+H)⁺ (ES+), 81.65%

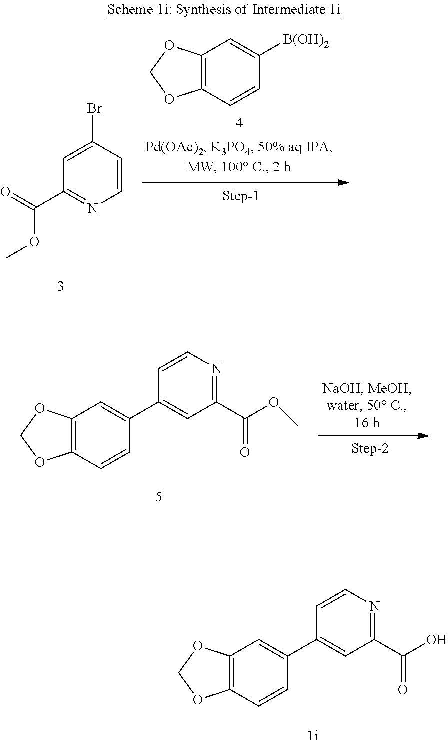 [SMILES: OB(O)c1ccc2c(c1)OCO2 ; COC(=O)c1cc(Br)ccn1 ; COC(=O)c1cc(-c2ccc3c(c2)OCO3)ccn1 ; O=C(O)c1cc(-c2ccc3c(c2)OCO3)ccn1]

Step-1 Synthesis of methyl 4-(benzo[d][1,3]dioxol-5-yl)picolinate (5): A solution of methyl 4-bromopicolinate (3, 100 mg, 0.46 mmol) and benzo[d][1,3]dioxol-5-ylboronic acid (4, 77 mg, 0.46 mmol) in propan-2-ol and water (10 mL, 1:1) was degassed with argon for 10 min. After degassing, palladium diacetate (31 mg, 0.13 mmol) and K₃PO₄ (295 mg, 1.39 mmol) were added. The reaction vial was sealed with aluminium cap and irradiated in microwave at 100° C. for 1 h. After completion of the reaction, the reaction was filtered through a pad of celite and washed with IPA (50 mL). Organic layers were combined, dried (Na₂SO₄) and concentrated in vacuo to give a crude mixture of methyl 4-(benzo[d][1,3]dioxol-5-yl)picolinate and 4-(benzo[d][1,3]dioxol-5-yl)picolinic acid (5 and 1i, 119 mg crude) as a black liquid. Crude was used for next step without purification. LCMS: m/z 258.26 (M+H)⁺ (ES+), 5% Int. 5 and 70% Int. 1i

Step-2 Synthesis of 4-(benzo[d][1,3]dioxol-5-yl)picolinic acid (1i): Crude mixture of methyl 4-(4-(difluoromethyl)phenyl)picolinate and 4-(benzo[d][1,3]dioxol-5-yl)picolinic acid (5 and 1i, 119 mg crude) was stirred in MeOH (5.0 mL) and sodium hydroxide (93 mg, 2.31 mmol) in water (1.00 mL) was added. The reaction mixture was stirred at 65° C. for 3 h. After completion of the reaction, reaction mixture was concentrated and water (20 mL) was added. Aqueous layer was extracted with ether (2×30 mL) and organic layer was separated. Aqueous layer was acidified with aq NaHSO₄ solution (10 mL). The aqueous layer was extracted with EtOAc (2×30 mL). The organic layers were combined, dried (Na₂SO₄) and concentrated in vacuo to give the title compound (1i, 110 mg) as a white solid. LCMS: m/z 244.28 (M+H)⁺ (ES+), 96.2%

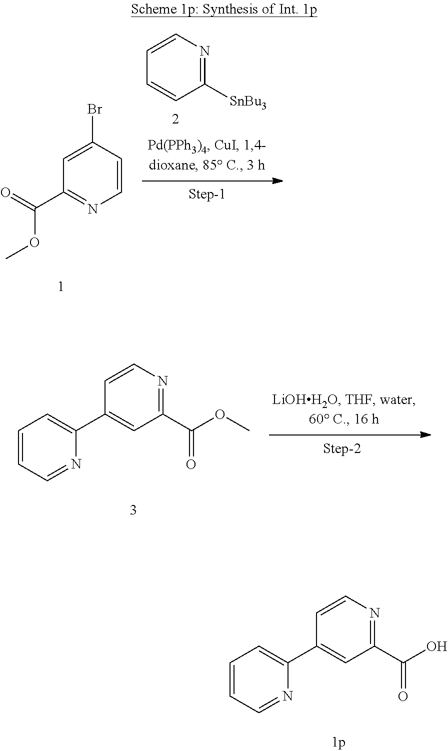

Step-1 Synthesis of methyl [2,4′-bipyridine]-2′-carboxylate (3): A solution of methyl 4-bromopicolinate (1, 1 g, 4.63 mmol) and 2-(tributylstannyl)pyridine (2, 1.70 g, 4.63 mmol) in dioxane (25.0 mL) was degassed with Argon for 10 min. Tetrakis (267 mg, 0.23 mmol) and CuI (88 mg, 0.46 mmol) were added to the reaction mixture and reaction vial was sealed with aluminium cap. The reaction mixture was stirred at 90° C. for 18 h. Progress of the reaction was monitored by TLC, which showed consumption of starting material. After completion of reaction, reaction mixture was diluted with water (50 mL) and the aqueous layer was extracted with EtOAc (2×100 mL). The organic layers were combined, dried (Na₂SO₄) and concentrated in vacuo. The residue was purified by flash column chromatography [silica gel (100-200 mesh), gradient 0% to 5% MeOH in DCM] to give a mixture of methyl [2,4′-bipyridine]-2′-carboxylate and [2,4′-bipyridine]-2′-carboxylic acid (3 and 4, 400 mg crude) as a brown solid which was used for further step without purification and analysis.

Step-2 Synthesis of [2,4′-bipyridine]-2′-carboxylic acid (1p): To a stirred a mixture 3 and 4 (160 mg, 0.74 mmol) in THF (10.0 mL) and water (3.00 mL), lithium hydroxide monohydrate (313 mg, 7.47 mmol) was added. The reaction mixture was stirred at 60° C. for 18 h. After completion of the reaction (monitored by TLC), reaction mixture was concentrated and water (20 mL) was added. Aqueous layer was extracted with ether (2×20 mL) and organic layer was separated. Aqueous layer was acidified with aq NaHSO₄ solution (10 mL). The aqueous layer was extracted with 10% MeOH in DCM (2×30 mL). The organic layers were combined, dried (Na₂SO₄) and concentrated in vacuo to give the title compound (1p, 150 mg crude) as a brown gum which was used for further step without purification and analysis.

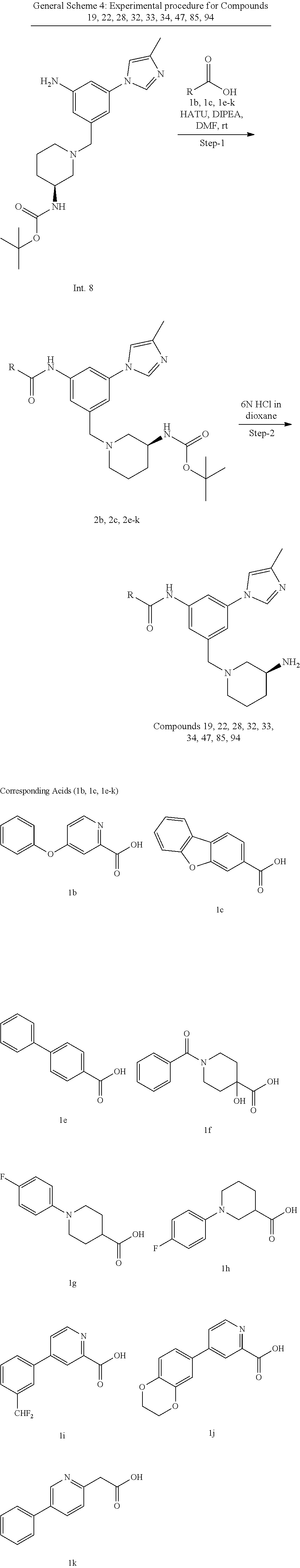

Ic is available commercially.

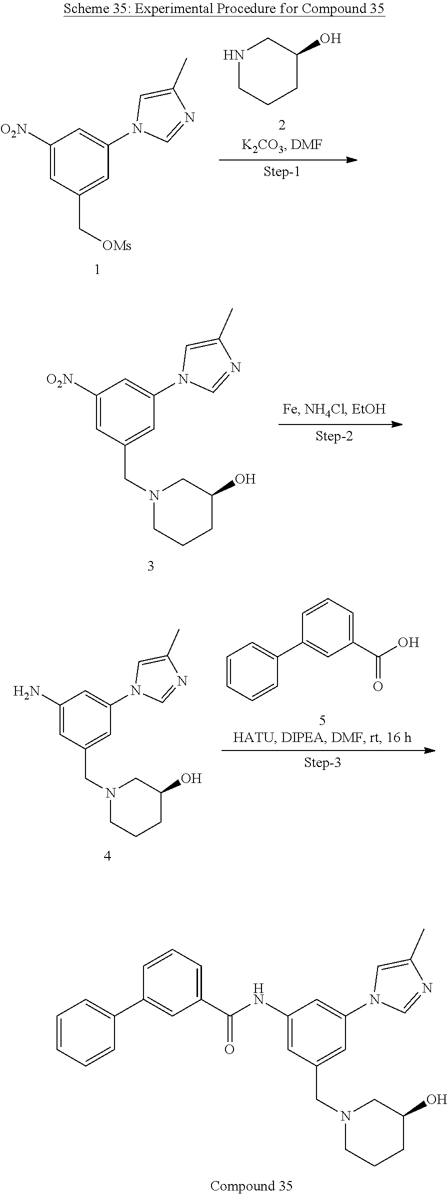

Step-1 Synthesis of (S)-1-(3-(4-methyl-1H-imidazol-1-yl)-5-nitrobenzyl)piperidin-3-ol (3): To a solution of (S)-piperidin-3-ol (2, 1.49 g, 14.72 mmol) in dry DMF (46 mL), K₂CO₃ (6.12 g, 44.3 mmol) was added. The reaction mass was stirred for 10 min and 3-(4-methyl-1H-imidazol-1-yl)-5-nitrobenzyl methanesulfonate (1, 4.6 g, 14.72 mmol) was added at room temperature. The resulting mixture was stirred for another 16 h. After completion of reaction (monitored by TLC), water (200 mL) was added to the reaction mixture and the aqueous layer was extracted with ethyl acetate (3×100 mL). The organic layers were combined, dried (Na₂SO₄) and concentrated in vacuo. The residue was purified by flash column chromatography [normal phase, silica gel (100-200 mesh), gradient 30% to 40% EtOAc in Hexane] to give the title compound (3, 1.92 g) as a brown gummy compound which was forwarded to next step without analysis.

Step-2 Synthesis of (S)-1-(3-amino-5-(4-methyl-1H-imidazol-1-yl)benzyl)piperidin-3-ol (4): A mixture of 3 (1.92 g, 63.0 mmol), NH₄Cl (2.03 g, 37.9 mmol) and Fe powder (3.14 g, 56.7 mmol) in EtOH: H₂O (40 ml, 6:1) were heated at 70° C. for 3 h. After complete consumption of the starting material (monitored by TLC), the reaction mixture was filtered through a pad of celite, washed with 10% MeOH in DCM (300 mL) and concentrated in vacuo. The residue was dissolved in 20% IPA/CHCl₃ (200 mL), washed with H₂O (50 mL). The combined organic layer was dried (Na₂SO₄) and concentrated in vacuo to give the title compound (4, 1.62 g, 92%) as a brown gummy compound. LCMS: m/z 287.31 (M+H)⁺ (ES+), 88%

Step-3 Synthesis of (S)—N-(3-((3-hydroxypiperidin-1-yl)methyl)-5-(4-methyl-1H-imidazol-1-yl)phenyl)-[1,1′-biphenyl]-3-carboxamide (Compound 35): To a solution of 4 (217 mg, 0.75 mmol) and acid 5 (150 mg, 1.50 mmol) in DMF (10 mL), DIPEA (0.39 mL, 1.89 mmol) was added followed by addition of HATU (267 mg, 2.25 mmol) at 0° C. The reaction mixture was stirred the at room temperature for 16 h. After completion of reaction, the reaction mixture was diluted with water (50 mL) and extracted with EtOAc (2×50 mL). The combined organic layer was dried (anhydrous Na₂SO₄) and concentrated in vacuo. The crude material was purified by prep-HPLC (reverse phase, Sunfire C18 (19×250) mm, 10 μm, gradient 38% to 62% ACN in water containing 0.1% TFA, RT: 8.79 min to give the title compound (Compound 35, 27 mg, 7.65%) as a white solid.

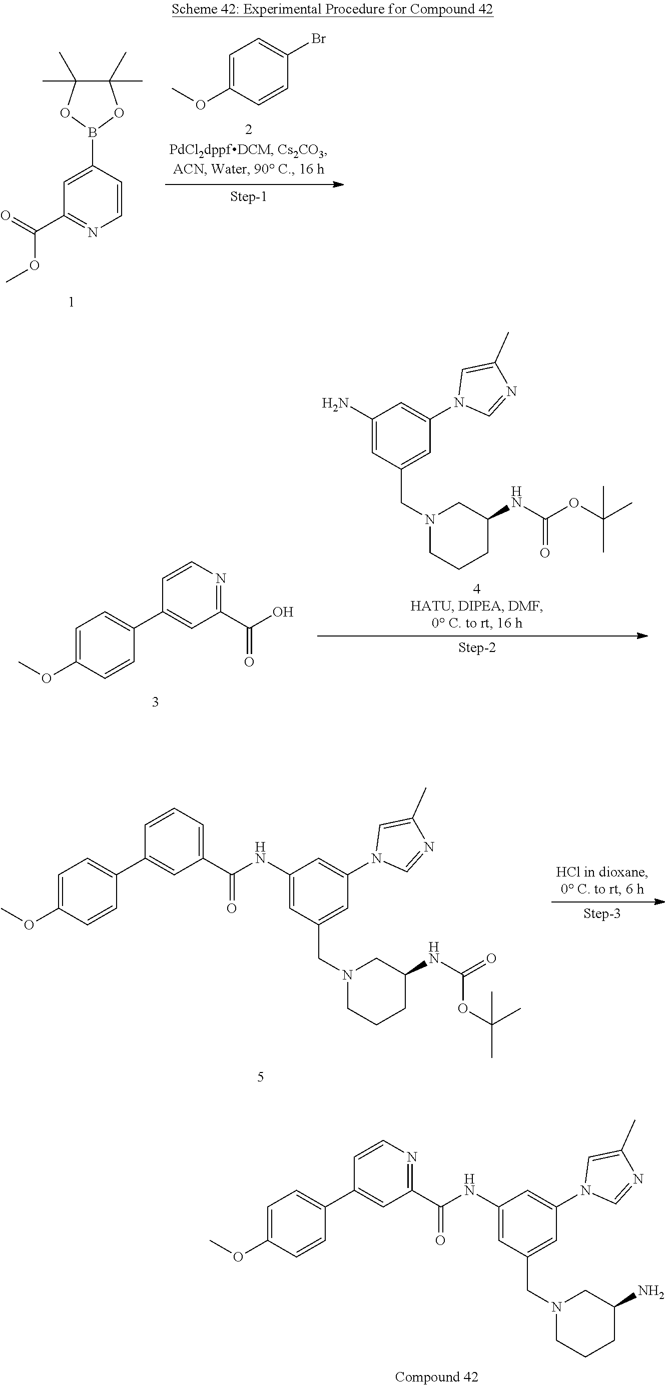

Step-1 Synthesis of 4-(4-methoxyphenyl)picolinic acid (3): To a stirred solution of methyl 4-(4,4,5,5-tetramethyl-1,3,2-dioxaborolan-2-yl)picolinate (1, 2.2 g, 10.2 mmol) and 1-bromo-4-methoxybenzene (2, 1.39 g, 9.70 mmol) in ACN (30 mL), caesium carbonate (6.6 g, 20.4 mmol) was added and reaction mass was degassed with N₂ for 10 min. PdCl₂(dppf)·DCM (0.74 g, 1.02 mmol) was added to the reaction mixture and stirred at 90° C. for 16 h. After completion (monitored by TLC), reaction mixture was diluted with water (50 mL) and extracted with EtOAc (2×50 ml). Aqueous layer was acidified with aq 1N HCl (10 mL). Brown precipitate was filtered off and wash with water (50 ml). The solid was dried under reduced pressure to give the title compound (3, 1.1 g, 47%) as a light-brown solid. LCMS: m/z 228.1 (M−H)⁻ (ES−), 97.58%

Step-2 Synthesis of tert-butyl (S)-(1-(3-(4-(4-methoxyphenyl)picolinamido)-5-(4-methyl-1H-imidazol-1-yl)benzyl)piperidin-3-yl)carbamate (5): To a solution of amine 4 (250 mg, 1.09 mmol) and acid 3 (500 mg, 1.31 mmol) in DMF (10 mL), DIPEA (422 mg, 3.27 mmol) was added followed by addition of HATU (622 mg, 1.64 mmol) at 0° C. and reaction mixture was stirred the at room temperature for 16 h. Progress of the reaction was monitored by TLC, which showed consumption of starting material. After completion of reaction, the reaction mixture was diluted with water (50 mL) and extracted with EtOAc (2×50 ml), the resulting organic layers was dried (Na₂SO₄) and concentrated in vacuo. The residue was purified by flash column chromatography [normal phase, silica gel (100-200 mesh), gradient 0% to 5% MeOH in DCM] to give the title compound (5, 180 mg, 27%) as a white solid. LCMS: m/z 597.23 (M+H)⁺ (ES+), 82.98%

Step-3 Synthesis of (S)—N-(3-((3-aminopiperidin-1-yl)methyl)-5-(4-methyl-1H-imidazol-1-yl)phenyl)-4-(4-methoxyphenyl)picolinamide (Compound 42): To a solution of 5 (0.18 g, 0.30 mmol) in DCM (4 mL), hydrogen chloride in dioxane (3.0 mL, 4M) was added and reaction mass was stirred the reaction mixture at rt for 6 h. Progress of the reaction was monitored by TLC, which showed consumption of starting material. After completion of reaction, the resulting reaction mixture was concentrated under reduced pressure. The crude material was purified by prep-HPLC (reverse phase, X Bridge shield C18 (19×250) mm, 10 μm, gradient 32% to 70% ACN in water containing ammonium bi-carbonate, RT: 9.78 min to give the title compound (Compound 42, 38 mg ˜11.8% acetate w/w present) as a yellow solid.

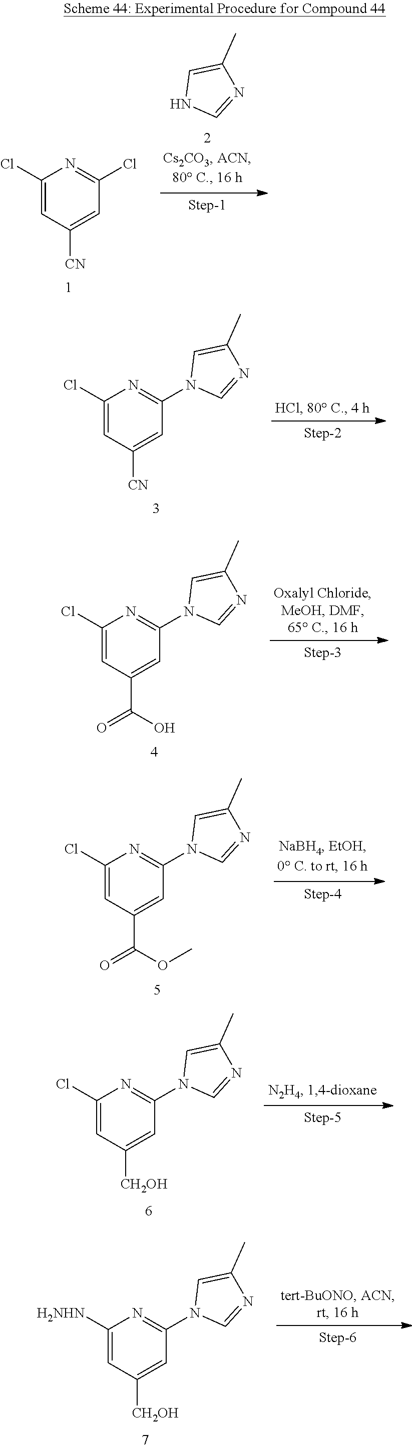

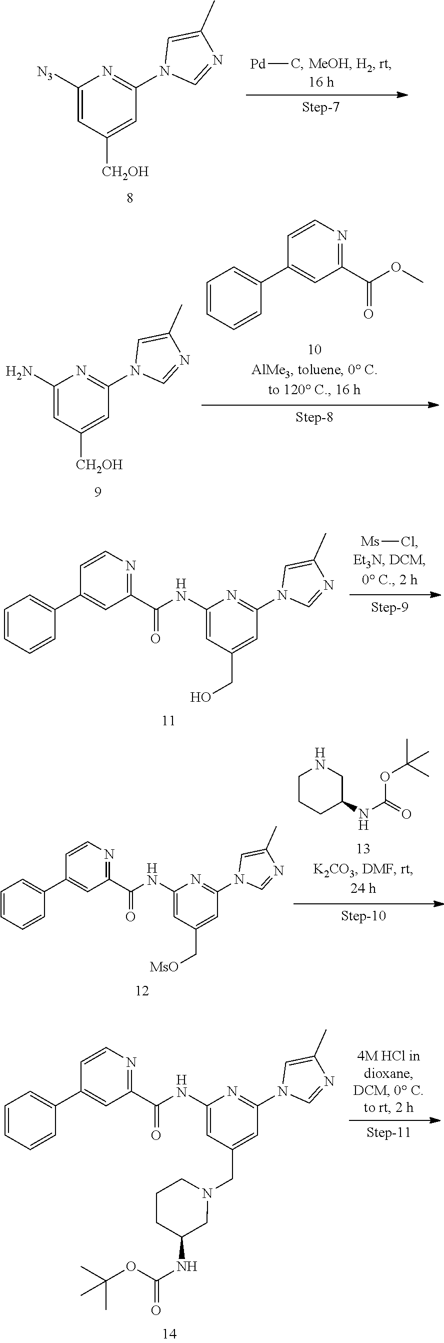

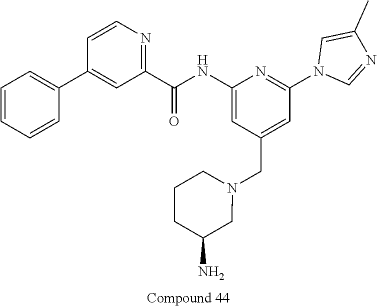 [SMILES: Cc1cn(-c2cc(CN3CCC[C@H](N)C3)cc(NC(=O)c3cc(-c4ccccc4)ccn3)n2)cn1]

Step-1 Synthesis of 2-chloro-6-(4-methyl-1H-imidazol-1-yl)isonicotinonitrile (3): To a stirred solution of 4-methyl-1H-imidazole (2, 3.76 g, 45.8 mmol) in ACN (100 mL), 052003 (24.9 g, 76.3 mmol) was added and the reaction mixture was stirred at room temperature for 30 min 2,6-dichloroisonicotinonitrile (1, 8.80 g, 50.9 mmol) was added to the reaction mixture and stirred at 80° C. for 16 h. After completion, water (200 mL) was added to the reaction mixture and the aqueous layer was extracted with ethyl acetate (3×50 mL). The organic layers were combined, dried (Na₂SO₄) and concentrated in vacuo. The residue was purified by flash column chromatography [normal phase, silica gel (100-200 mesh), gradient 0% to 1% MeOH in DCM] to give the title compound (3, 4.10 g, 36.8%) as an off-white solid. LCMS: m/z 218.98 (M+H)⁺ (ES+), 78.3%

Step-2 Synthesis of 2-chloro-6-(4-methyl-1H-imidazol-yl)isonicotinic acid (4): 3 (8.40 g, 38.4 mmol) was stirred in conc. HCl (100 mL) at 80° C. for 4 h. After completion of reaction (monitored by TLC), the resulting reaction mixture was cool to 25° C. and concentrated under reduced pressure to give the title compound (4, 8.10 g, 88.7%) as an off-white solid. LCMS: m/z 238.24 (M+H)⁺ (ES+), 81.86%

Step-3 Synthesis of methyl 2-chloro-6-(4-methyl-1H-imidazol-1-yl)isonicotinate (5): To the stirred solution of acid 4 (2.00 g, 8.42 mmol) in methanol (50.0 mL), oxalic dichloride (2.17 mL, 25.2 mmol) and DMF (100 μL) was added and reaction mixture was stirred at 65° C. for 16 h. After completion (monitored by TLC), reaction mixture was concentrated under reduced pressure. Reaction mixture was diluted with NaHCO₃ solution (40 mL) and aqueous layer was extracted with EtOAc (2×60 mL). Organic layer was collected, dried over anhydrous sodium sulfate and concentrated under reduced pressure to give the title compound (5, 2.00 g, 94.4%) as a white solid. LCMS: m/z 251.81 (M+H)⁺ (ES+), 86.30% ¹H-NMR (400 MHz; CDCl₃): δ 2.39 (s, 3H), 4.03 (s, 3H), 7.53 (s, 1H), 7.82-7.87 (m, 2H), 8.63 (s, 1H).

Step-4 Synthesis of (2-chloro-6-(4-methyl-1H-imidazol-1-yl)pyridin-4-yl)methanol (6): To the stirred solution of 5 (1.70 g, 6.75 mmol) in ethanol (50.0 mL), sodium borohydride (968 mg, 4 eq., 27.0 mmol) was added at 0° C. and reaction mixture was stirred at room temperature for 2 h. After completion (monitored by TLC), reaction mixture was concentrated under reduced pressure. Reaction mixture was diluted with water (10 mL) and aqueous layer was extracted with EtOAc (2×30 mL). Organic layers were collected, dried over anhydrous sodium sulfate and concentrated under reduced pressure to give the title compound (6, 1.45 g, 95.9%) as a white solid. LCMS: m/z 224.30 (M+H)⁺ (ES+), 80.2%

Step-5 Synthesis of (2-hydrazineyl-6-(4-methyl-1H-imidazol-1-yl)pyridin-4-yl)methanol (7): To 6 (1.40 g, 6.26 mmol), hydrazine hydrate (50 mL) was added and stirred at 100° C. for 16 h. After reaction completion (monitored by TLC), reaction mixture was concentrated under reduced pressure to give the title compound (7, 2.0 g crude) as an off-white gum. Crude was used for next step without purification. LCMS: m/z 220.39 (M+H)⁺ (ES+), 71.3%

Step-6 Synthesis of (2-azido-6-(4-methyl-1H-imidazol-1-yl)pyridin-4-yl)methanol (8): To the stirred solution 7 (2.00 g crude, 9.12 mmol) in acetonitrile (30.0 mL), tert-butyl nitrite (21.9 mL, 182 mmol) was added and stirred at room temperature for 16 h. After reaction completion (monitored by TLC), reaction mixture was concentrated under reduced pressure to give the title compound (8, 2.10 g crude) as a yellow gummy material which was used for next step without purification. LCMS: m/z 231.32 (M+H)⁺ (ES+), 56.6%

Step-7 Synthesis of (2-amino-6-(4-methyl-1H-imidazol-1-yl)pyridin-4-yl)methanol (9): To the stirred solution of 8 (2.10 g crude, 9.12 mmol) in methanol (50.0 mL), 10% palladium on carbon (1.5 g, 50% wet) was added and reaction mixture was stirred in the presence of H₂ (1 atm) at room temperature for 16 h. The reaction mixture was filtered through a pad of celite and washed with MeOH (2×50 mL). The filtrate was concentrated in vacuo. The crude was triturated with ether (50 mL) and ether was decanted. The solid was dried under reduced pressure to give the title compound (9, 2.10 g crude) as a brown solid which was used for next step without purification. LCMS: m/z 205.01 (M+H)⁺ (ES+), 52.2%

Step-8 Synthesis of N-(4-(hydroxymethyl)-6-(4-methyl-1H-imidazol-1-yl)pyridin-2-yl)-4-phenyl picolinamide (11): To the stirring solution of 9 (300 mg, 1.47 mmol) in toluene (8.00 mL), trimethylaluminum (2.94 mL, 5.88 mmol) was added at 0° C. The reaction mixture was stirred at room temperature for 20 min and methyl 4-phenylpyridine-2-carboxylate (10, 157 mg, 0.73 mmol) in toluene (2 mL) was added at 0° C. The resulting mixture was stirred at 120° C. for 16 h. After reaction completion (monitored by TLC), reaction mixture was diluted with water (30 mL) and aqueous layer was extracted with DCM (2×30 mL). Organic layers were collected, dried over anhydrous sodium sulfate and concentrated under reduced pressure. The residue was purified by flash column chromatography [normal phase, silica gel (100-200 mesh), gradient 50% to 70% EtOAc in Hexane] to give the title compound (11, 80.0 mg, 11.8%, over three steps) as a yellow solid. LCMS: m/z 386.11 (M+H)⁺ (ES+), 83.2%

Step-9: Synthesis of (2-(4-methyl-1H-imidazol-1-yl)-6-(4-phenylpicolinamido)pyridin-4-yl)methyl methanesulfonate (12): To a solution of 11 (75.0 mg, 0.19 mmol) in DCM (4 mL), triethylamine (0.08 mL, 0.58 mmol) was added drop wise. After 15 min stirring at room temperature, methanesulfonyl chloride (0.02 mL, 0.29 mmol) was slowly added at 0° C. and the reaction was stirred at same temperature for 2 h. After completion (monitored by TLC), the reaction mixture was quenched with water (30 mL) and extracted with DCM (2×30 mL). The organic layers were combined, dried (Na2SO4) and concentrated in vacuo to give the title compound (12, 110 mg crude) as a brown gummy liquid which was used for the next step without purification.

Step-10: Synthesis of tert-butyl (S)-(1-((2-(4-methyl-1H-imidazol-1-yl)-6-(4-phenylpicolinamido)pyridin-4-yl)methyl)piperidin-3-yl)carbamate (13): To a solution of 13 (156 mg, 0.77 mmol) in dry DMF (4 mL), K₂CO₃ (80.5 mg, 0.58 mmol) was added to it. The reaction mixture was stirred for 10 min and 12 (90.0 mg, 0.19 mmol) was added. The reaction mixture was stirred at room temperature for 16 h. After consumption of starting material (monitored by TLC), the reaction mixture was diluted with water (30 mL), yellow solid precipitates observed which was extracted with EtOAc (2×50). The combined organic layer was washed with bine (20 mL), dried with Na₂SO₄ and concentrated in vacuo to get crude material. The residue was purified by flash column chromatography [normal phase, silica gel (100-200 mesh), gradient 0% to 5% MeOH in DCM] to give the title compound (13, 110 mg crude) as a yellow semi-solid. LCMS: m/z 568.67 (M+H)⁺ (ES+), 60.19%

Step-11: Synthesis of (S)—N-(4-((3-aminopiperidin-1-yl)methyl)-6-(4-methyl-1H-imidazol-1-yl)pyridin-2-yl)-4-phenylpicolinamide (Compound 44): To a stirred solution of 13 (110 mg crude, 0.19 mmol) in dichloromethane (5 mL), hydrogen chloride in dioxane (4.0 mL, 4M) was added at 0° C. and stirred the reaction mixture at room temperature for 2 h. After completion of the reaction, the reaction mixture was concentrated in vacuo. The residue was purified by prep-HPLC (reverse phase, X Bridge shield C18 (19×250) mm, 10 μm, gradient 25% to 70% ACN in water containing 5 mM Ammonium bicarbonate, RT: 11.89 min to give the title compound (Compound 44, 7.70 mg, 8.48%, over two steps) as a light-pink solid.

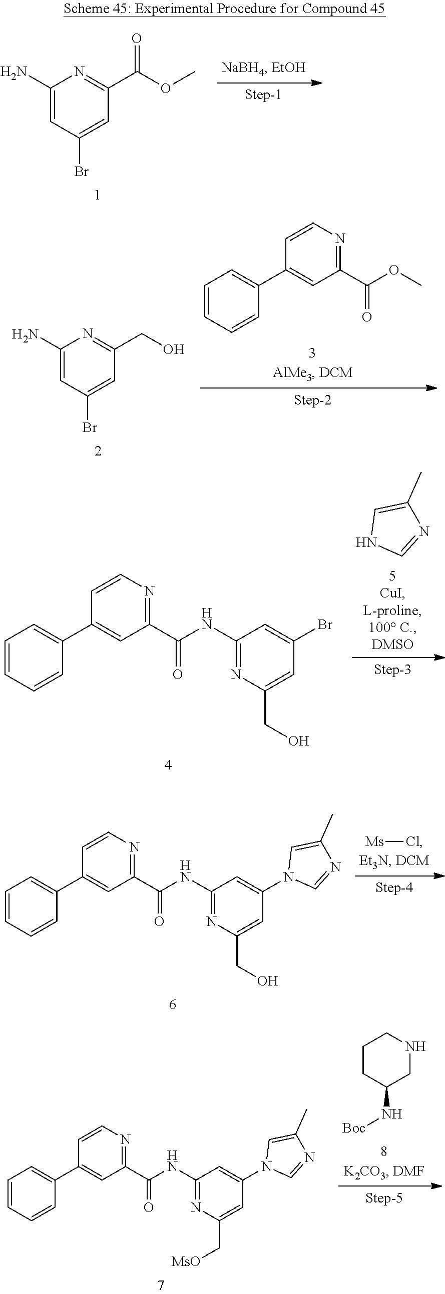

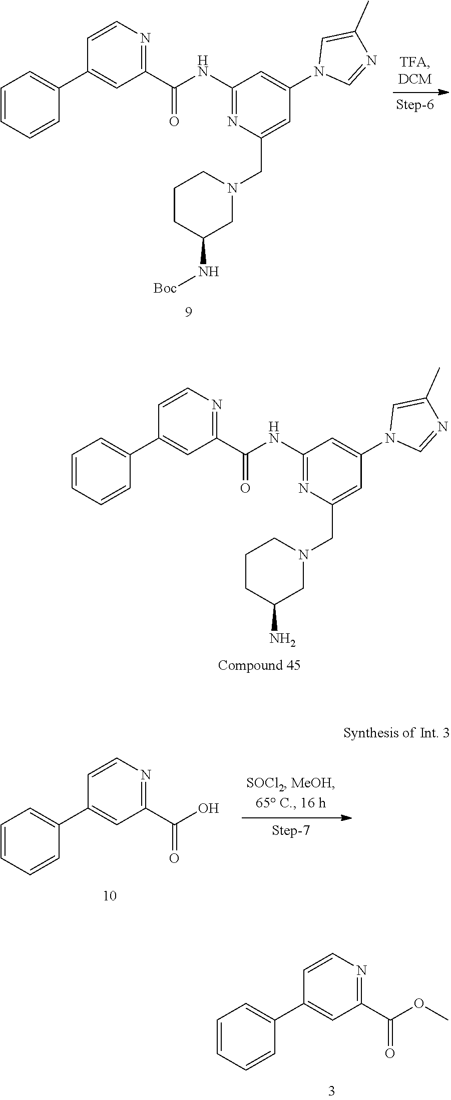 [SMILES: Cc1cn(-c2cc(CN3CCC[C@H](NC(=O)OC(C)(C)C)C3)nc(NC(=O)c3cc(-c4ccccc4)ccn3)c2)cn1 ; Cc1cn(-c2cc(CN3CCC[C@H](N)C3)nc(NC(=O)c3cc(-c4ccccc4)ccn3)c2)cn1 ; O=C(O)c1cc(-c2ccccc2)ccn1 ; COC(=O)c1cc(-c2ccccc2)ccn1]

Step-1 Synthesis of (6-amino-4-bromopyridin-2-yl)methanol (2): To the stirred solution of 1 (250 mg, 1.08 mmol) in ethanol (6.00 mL), sodium borohydride (205 mg, 5.41 mmol) was added at 0° C. and the reaction mixture was stirred at room temperature for 2 h. After completion of reaction (monitored by TLC), reaction mixture was concentrated under reduced pressure. Reaction mixture was diluted with water (10 mL) and aqueous layer was extracted with DCM (2×30 mL). Organic layer was collected, dried over anhydrous sodium sulfate and concentrated under reduced pressure to give the title compound (2, 240 mg crude) as a white solid. LCMS: m/z 203.23 (M+H)⁺ (ES+), 97.56%

Step-2 Synthesis of N-(4-bromo-6-(hydroxymethyl)pyridin-2-yl)-4-phenylpicolinamide (4): To the stirring solution of 2 (240 mg, 1.18 mmol) in DCM (10.00 mL), trimethylalumane (2.96 mL, 5.91 mmol) was added at 0° C. and reaction mixture was stirred at room temperature for 20 min. Methyl 4-phenylpyridine-2-carboxylate (3, 252 mg, 1.18 mmol) in DCM (2 mL) was added at 0° C. to the reaction mixture and reaction mixture was stirred at room temperature for 16 h. After completion (monitored by TLC), reaction mixture was diluted with water (20 mL) and aqueous layer was extracted with DCM (2×50 mL). Organic layers were collected, dried over anhydrous sodium sulphate and concentrated under reduced pressure. The residue was purified by flash column chromatography [normal phase, silica gel (100-200 mesh), gradient 50% to 70% EtOAc in Hexane] to give the title compound (4, 132 mg, 29.06%, over two steps) as a yellow solid. LCMS: m/z 384.02 (M+H)⁺ (ES+), at 2.25 min, 96.89%

Step-3 Synthesis of N-(6-(hydroxymethyl)-4-(4-methyl-1H-imidazol-1-yl)pyridin-2-yl)-4-phenylpicolinamide (6): To the solution of 4 (105 mg, 0.27 mmol) and 4-methyl-1H-imidazole (5, 90 mg, 1.09 mmol), argon gas was purged for 10 min. copper iodide (42 mg, 0.22 mmol), dipotassium carbonate (94.0 mg, 0.68 mmol) and L-proline (16 mg, 0.14 mmol) were added to the reaction mixture. The reaction vial was sealed and stirred at 100° C. for 16 h. After completion (monitored by TLC), reaction mixture was diluted with water (30 mL) and solid was filtered. The solid was dried under reduced pressure to give the title compound (6, 220 mg crude) as a light-green solid. LCMS: m/z 386.40 (M+H)⁺ (ES+), at 2.01 min, 77.22%

Step-4 Synthesis of (4-(4-methyl-1H-imidazol-1-yl)-6-(4-phenylpicolinamido)pyridin-2-yl)methyl methanesulfonate (7): To a solution of 6 (220 mg, 0.57 mmol) in DCM (5 mL), triethylamine (0.33 mL, 2.28 mmol) was added drop wise. After 15 min stirring at room temperature, methanesulfonyl chloride (0.09 mL, 1.14 mmol) was slowly added at 0° C. for 1 h. After completion (monitored by TLC), the reaction mixture was quenched with water (30 mL) and extracted with DCM (2×50 mL). The organic layers were combined, dried (Na₂SO₄) and concentrated in vacuo to give the title compound (7, 265 mg crude) as a brown gummy liquid which was used for the next step without purification.

Step-5 Synthesis of tert-butyl (S)-(1-((4-(4-methyl-1H-imidazol-1-yl)-6-(4-phenylpicolinamido)pyridin-2-yl)methyl)piperidin-3-yl)carbamate (9): To a solution of 8 (114 mg, 0.57 mmol) in dry DMF (5 mL), K₂CO₃ (237 mg, 0.58 mmol) was added to it. The reaction mixture was stirred for 15 min and 7 (265 mg, 0.57 mmol) was added. The reaction mixture was stirred at room temperature for 16 h. After consumption of starting material (monitored by TLC), the reaction mixture was diluted with water (30 mL), yellow solid precipitates observed which was filtered. The solid was washed with water (10 mL) and dried under reduced pressure to give the title compound (9, 324 mg crude) as a yellow solid. This crude was used for the next step without purification. LCMS: m/z 568.46 (M+H)⁺ (ES+), at 2.38 min, 76.06%

Step-6 Synthesis of (S)—N-(6-((3-aminopiperidin-1-yl)methyl)-4-(4-methyl-1H-imidazol-1-yl)pyridin-2-yl)-4-phenylpicolinamide (Compound 45): To a stirred solution of 9 (324 mg, 0.57 mmol) in dichloromethane (8.00 mL), hydrogen chloride in dioxane (12.0 mL, 4M) was added at 0° C. and stirred the reaction mixture at room temperature for 1 h. After completion of the reaction, the reaction mixture was concentrated in vacuo. The residue was purified by prep-HPLC (reverse phase, T3 C18 (19×250) mm, 10 μm, gradient 30% to 70% ACN in water containing 0.1% TFA, RT: 12.18 min to give the title compound (Compound 45, 20 mg, 7.49%, over three steps) as an off-white gum.

Step-7 Synthesis of methyl 4-phenylpicolinate (3): To a stirred solution of 4-phenylpyridine-2-carboxylic acid (10, 285 mg, 1.43 mmol) in methanol (15.0 mL), thionyl chloride (0.52 mL, 7.15 mmol) was added and stirred at 65° C. for 16 h. After completion (monitored by TLC), reaction mixture was concentrated under reduced pressure. Reaction mixture was diluted with NaHCO₃ solution (20 mL) and aqueous layer was extracted with EtOAc (2×40 mL). Organic layer was collected, dried over anhydrous sodium sulfate and concentrated under reduced pressure to give the title compound (3, 300 mg, 98.3%) as a white gum. LCMS: m/z 214.32 (M+H)⁺ (ES+), 99.38%

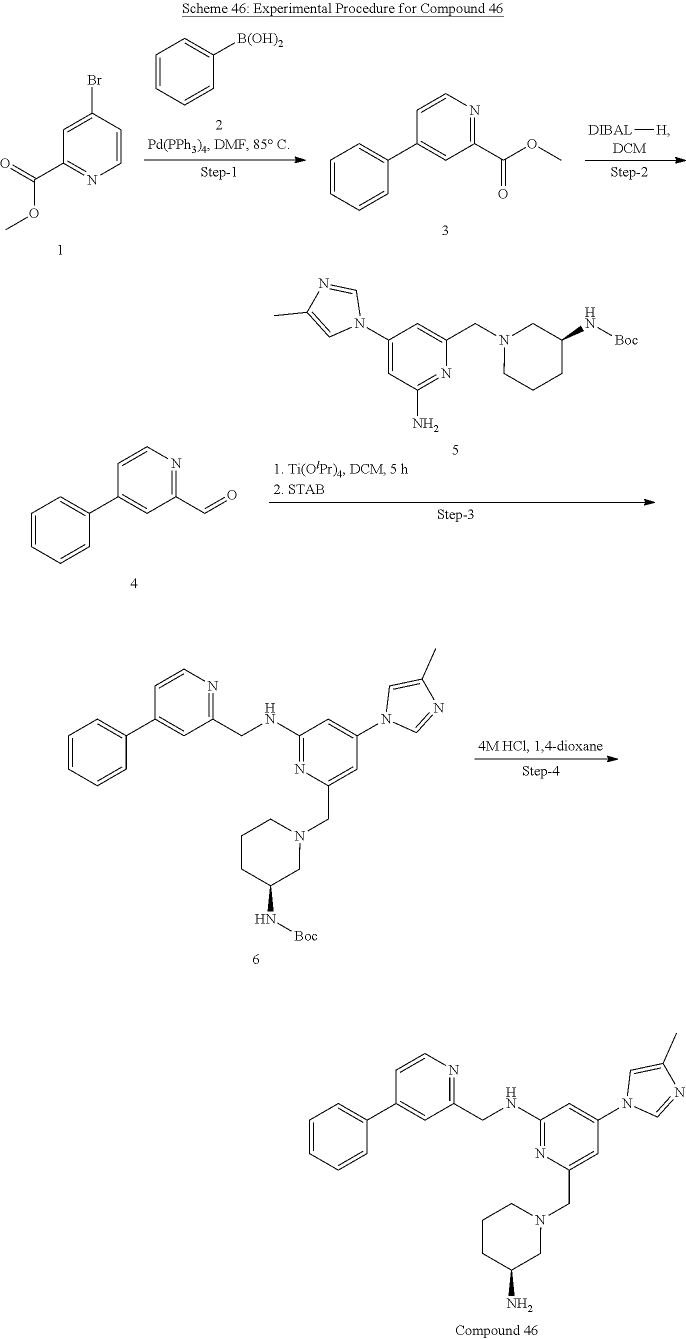

Step-1 Synthesis of methyl 4-phenylpyridine-2-carboxylate (3): To a mixture of methyl 4-bromopyridine-2-carboxylate (1, 1 g, 0.462 mmol) and phenylboronic acid (2, 0.564 g, 0.462 mmol) in Toluene:H₂O:EtOH (3:1:5, 9 mL), sodium carbonate (1.27 g, 9.22 mmol) was added. The reaction mixture was degassed with N₂ for 10 minutes, Pd(PPh₃)₄ (0.169 g, 0.0231 mmol) was added to it. The resulting reaction mixture was sealed with aluminium cap and stirred at 80° C. for 18 h. Progress of the reaction was monitored by TLC, which showed consumption of starting material. After completion of reaction, the reaction mixture was filtered through a pad of celite and washed with EtOAc (2×30 mL). Organic layers were combined, dried (Na₂SO₄) and concentrated in vacuo. The residue was purified by flash column chromatography [silica gel (100-200 mesh), gradient 10% to 20% EtOAc in Hexane] to give the title compound (3, 803 mg crude) as a colourless gum. LCMS: m/z 213.89 (M+H)⁺ (ES+), 80.21%

Step-2 Synthesis of 4-phenyl-2-pyridinaldehyde (4): To a stirred solution of 3 (0.4 g, 1.89 mmol) in dry THF (6.0 mL), DIBAL-H (5.63 mL, 5.63 mmol, 1 M in toluene) at −78° C. and reaction mixture was stirred at same temperature for 2 h. Progress of the reaction was monitored by TLC, which showed consumption of starting material. After completion of reaction, the reaction mixture was quenched with ammonium chloride solution and filter through a pad of celite and washed with EtOAc (2×30 mL). The reaction mixture was diluted with water (20 mL) and the aqueous layer was extracted with EtOAc (2×40 mL). The combined organic layer was dried (Na₂SO₄) and concentrated in vacuo to give the title compound (4, 300 mg crude) as a yellowish liquid. LCMS: m/z 184.11 (M+H)⁺ (ES+), 76.42%

Step-3 Synthesis of tert-butyl (S)-(1-((4-(4-methyl-1H-imidazol-1-yl)-6-(((4-phenylpyridin-2-yl)methyl) amino) pyridin-2-yl)methyl)piperidin-3-yl)carbamate (6): To a solution of 4 (631 mg, 1.63 mmol) and 4-phenylpicolinaldehyde (4, 300 mg, 1.63 mmol) in DCM (20 mL), Ti(OⁱPr)₄ (1.45 mL, 4.91 mmol) was added at 0° C. and the reaction mixture was stirred for 5 h then Na(OAc)₃BH (1.04 g, 4.91 mmol) was added portion wise to the reaction mixture and stirred at 0° C. for 6 h. After completion, the reaction mixture was quenched with aq sat NaHCO₃ and extracted with DCM (3×30 mL). The organic layers were combined and washed with brine, dried (Na₂SO₄) and concentrated in vacuo. The residue was purified by flash column chromatography [silica gel (100-200 mesh), gradient 1% to 2% MeOH in DCM] to give the title compound 6 (500 mg) as a brown yellow solid which was used further without analysis.

Step-4 Synthesis of (S)-6-((3-aminopiperidin-1-yl)methyl)-4-(4-methyl-1H-imidazol-1-yl)-N-((4-phenylpyridin-2-yl)methyl)pyridin-2-amine (Compound 46): To a solution of 6 (230 mg, 0.416 mmol) in DCM (5 mL), 4 M HCl in dioxane (10 mL) was added. After completion (monitored by TLC), the reaction mixture was concentrated in vacuo. The crude material was purified by prep-HPLC (reverse phase, X Bridge Shield C18 (19×250) mm, 10 μm, gradient 50% to 50% ACN in water containing 5 mM Ammonium acetate, RT: 12.28 min to give the title compound (Compound 46, 61 mg, 31%) as an off-white solid.

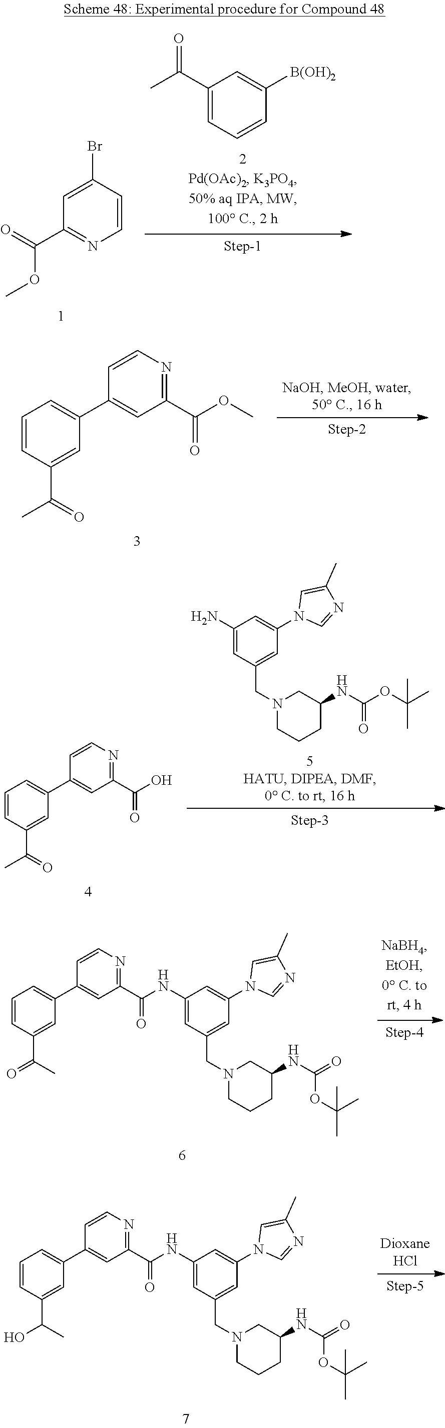

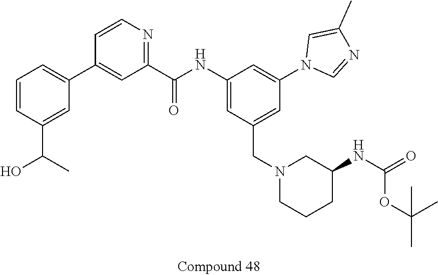 [SMILES: Cc1cn(-c2cc(CN3CCC[C@H](NC(=O)OC(C)(C)C)C3)cc(NC(=O)c3cc(-c4cccc(C(C)O)c4)ccn3)c2)cn1]

Step-1 Synthesis of methyl 4-(3-acetylphenyl)picolinate (3): A solution of 1 (1 g, 4.63 mmol) and (3-acetylphenyl)boronic acid (2, 759 mg, 4.63 mmol) in propan-2-ol and water (40 mL, 1:1) was degassed with argon for 10 min. After degassing, palladium diacetate (416 mg, 1.85 mmol) and tripotassium phosphate (2.95 g, 13.9 mmol) were added. The reaction vial was sealed with aluminium cap and irradiated in microwave at 100° C. for 1 h. After completion of the reaction (monitored by TLC), the reaction was filtered through a pad of celite and washed with IPA (50 mL). Organic layers were combined, dried (Na₂SO₄) and concentrated in vacuo to give the title compound (3, 800 mg crude) as a black liquid which was used for next step without further purification.

Step-2 Synthesis of 4-(3-acetylphenyl)picolinic acid (4): To a stirred solution of 3 (800 mg crude) in THF (10.0 mL) and methanol (10 mL), sodium hydroxide (376 mg) in water (5 mL) was added. The reaction mixture was stirred at 60° C. for 16 h. After completion of the reaction (monitored by TLC), reaction mixture was concentrated and water (20 mL) was added. Aqueous layer was extracted with ether (2×20 mL) and organic layer was separated. Aqueous layer was acidified with 5% aq NaHSO₄ solution and aqueous layer was extracted with ethyl acetate (2×30 mL). The organic layers were combined, dried (Na₂SO₄) and concentrated in vacuo to give the title compound (4, 400 mg crude) as a white solid which was used for next step without purification. LCMS: m/z 242.29 (M+H)⁺ (ES+), 54.96%

Step-3 Synthesis of tert-butyl (S)-(1-(3-(4-(3-acetylphenyl)picolinamido)-5-(4-methyl-1H-imidazol-1-yl)benzyl)piperidin-3-yl)carbamate (6): To a solution of 4 (250 mg, 0.57 mmol) in DMF (10.00 mL), DIPEA (0.76 mL, 4.15 mmol) and HATU (512 mg, 1.35 mmol) were sequentially added. After 10 min stirring at same temperature 5 (200 mg, 0.52 mmol) was added and the resulting mixture was stirred at room temperature for 16 h. After completion of the reaction (monitored by TLC), water (20 mL) was added to the reaction mixture and the aqueous layer was extracted with ethyl acetate (2×30 mL). The organic layers were combined, dried (Na₂SO₄) and concentrated in vacuo to give the title compound (6, 400 mg crude) as a yellow semi-solid which was used for next step without purification. LCMS: m/z 609.44 (M+H)⁺ (ES+), at 2.22 min, 31.6%

Step-4 Synthesis of tert-butyl ((3S)-1-(3-(4-(3-(1-hydroxyethyl)phenyl)picolinamido)-5-(4-methyl-1H-imidazol-1-yl)benzyl)piperidin-3-yl)carbamate (7): To a stirred solution of 6 (400 mg, 0.20 mmol) in methanol (10.0 mL), sodium borohydride (235 mg, 6.57 mmol) was added at 0° C. and stirred the reaction mixture at room temperature for 4 h. After completion of the reaction (monitored by TLC), reaction mixture was concentrated under reduced pressure. The reaction mixture was quenched with water (20 mL) and aqueous layer was extracted with ethyl acetate (2×50 mL). The organic layers were combined, dried (Na₂SO₄) and concentrated in vacuo. The residue was subjected to flash column chromatography to enrich the purity to 58.8% [normal phase, silica gel (100-200 mesh), gradient 0% to 5% MeOH in DCM] to give the title compound (7, 100 mg) as a yellowish gum. LCMS: m/z 611.49 (M+H)⁺ (ES+), at 2.14 min, 58.8%

Step-5 Synthesis of N-(3-(((S)-3-aminopiperidin-1-yl)methyl)-5-(1H-imidazol-1-yl)phenyl)-4-(3-(1-hydroxyethyl)phenyl)picolinamide (Compound 48): To a solution of 7 (100 mg, 0.096 mmol) DCM (10 mL), HCl in dioxane (10 mL, 4M) was added. After completion of reaction (monitored by TLC), the reaction mixture was concentrated in vacuo. The residue was purified by prep-HPLC (reverse phase, T3 C18 (19×250) mm, 10 μm, gradient 30% to 60% ACN in water containing 0.1% TFA, RT: 15.18 min to give the title compound (Compound 48, 17 mg, 20%, over 4 steps) as an off-white semi-solid.

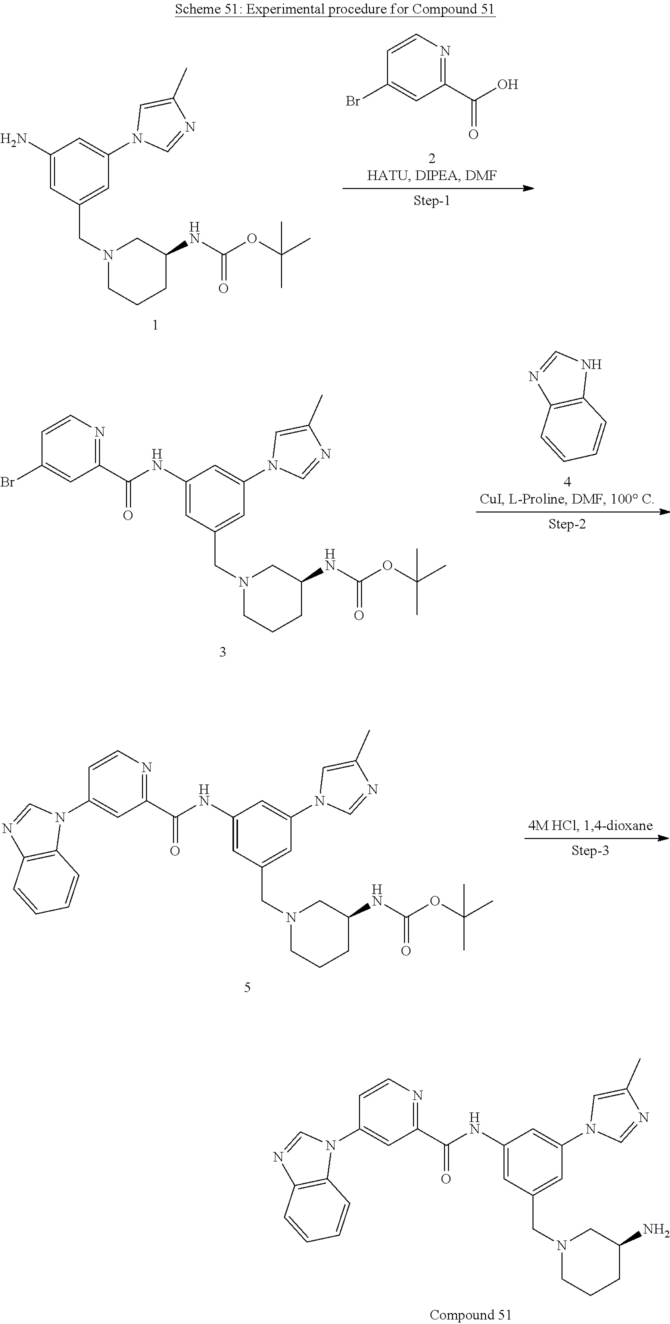 [SMILES: Cc1cn(-c2cc(N)cc(CN3CCC[C@H](NC(=O)OC(C)(C)C)C3)c2)cn1 ; O=C(O)c1cc(Br)ccn1 ; Cc1cn(-c2cc(CN3CCC[C@H](NC(=O)OC(C)(C)C)C3)cc(NC(=O)c3cc(Br)ccn3)c2)cn1 ; c1ccc2[nH]cnc2c1 ; Cc1cn(-c2cc(CN3CCC[C@H](NC(=O)OC(C)(C)C)C3)cc(NC(=O)c3cc(-n4cnc5ccccc54)ccn3)c2)cn1 ; Cc1cn(-c2cc(CN3CCC[C@H](N)C3)cc(NC(=O)c3cc(-n4cnc5ccccc54)ccn3)c2)cn1]

Step-1 Synthesis of tert-butyl (S)-(1-(3-(4-bromopicolinamido)-5-(4-methyl-1H-imidazol-1-yl)benzyl)piperidin-3-yl)carbamate (3): To a solution of 4-bromopicolinic acid (2, 300 mg, 1.49 mmol) in DMF (5.00 mL), DIPEA (0.65 mL, 3.71 mmol) and HATU (847 mg, 2.23 mmol) were sequentially added. After 10 min stirring at same temperature, amine 1 (344 mg, 0.89 mmol) was added and the resulting mixture was stirred at room temperature for 16 h. After completion of the reaction (monitored by TLC), water (20 mL) was added to the reaction mixture and the aqueous layer was extracted with DCM (2×50 mL). The organic layers were combined, dried (Na₂SO₄) and concentrated in vacuo. The residue was purified by flash column chromatography [normal phase, silica gel (100-200 mesh), gradient 0% to 5% MeOH in DCM] to give the title compound (3, 300 mg, 35%) as a brown solid.

Step-2 Synthesis of tert-butyl (S)-(1-(3-(4-(1H-benzo[d]imidazol-1-yl)picolinamido)-5-(4-methyl-1H-imidazol-1-yl)benzyl)piperidin-3-yl)carbamate (5): To a stirred solution of 3 (300 mg, 0.52 mmol) and 1H-benzo[d]imidazole (4, 218 mg, 1.84 mmol) in DMF, Argon gas was purged for 10 min. copper iodide (80 mg, 0.42 mmol), dipotassium carbonate (182 mg, 1.32 mmol) and L-proline (30 mg, 0.26 mmol) were added to the reaction mixture. The reaction vial was sealed and stirred at 110° C. for 16 h. After completion (monitored by TLC), reaction mixture was diluted with water (30 mL) and solid was filtered. The solid was dried under reduced pressure to give the title compound (5, 300 mg crude) as a light-brown semi-solid. LCMS: m/z 607.65 (M+H)⁺ (ES+), 48%

Step-3 Synthesis of (S)—N-(3-((3-aminopiperidin-1-yl)methyl)-5-(4-methyl-1H-imidazol-1-yl)phenyl)-4-(1H-benzo[d]imidazol-1-yl)picolinamide (Compound 51): To a solution of 15 (300 mg, crude) in DCM (10 mL), 4M HCl in dioxane (15.0 mL) was added. After completion of reaction (monitored by TLC), the reaction mixture was concentrated in vacuo The crude material was purified by prep-HPLC (reverse phase, X Bridge shield C18 (19×250) mm, 10 μm, gradient 92% ACN in water containing 5 mM Ammonium acetate, RT: 16.19 to give the title compound (Compound 51, 18.0 mg) as a white solid.

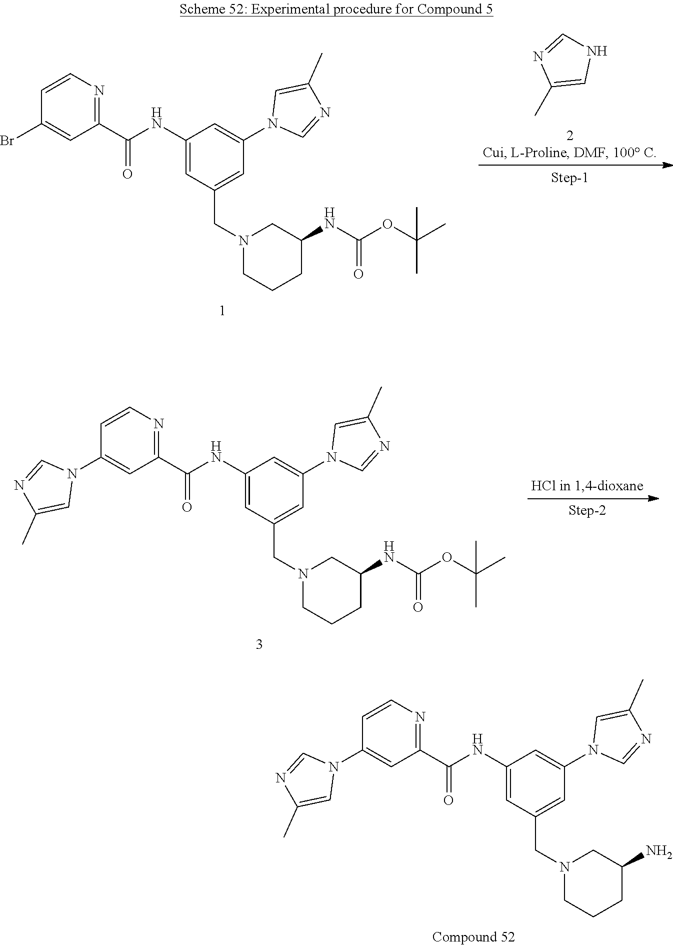

Step-1 Synthesis of tert-butyl (S)-(1-(3-(4-bromopicolinamido)-5-(4-methyl-1H-imidazol-1-yl)benzyl)piperidin-3-yl)carbamate (3): To the solution of 1 (200 mg, 0.35 mmol) and 4-methyl-1H-imidazole (2, 101 mg, 1.23 mmol) in DMF, Argon gas was purged for 10 min. copper iodide (54 mg, 0.28 mmol), dipotassium carbonate (121 mg, 0.88 mmol) and L-proline (20 mg, 0.18 mmol) were added to the reaction mixture. The reaction vial was sealed and stirred at 110° C. for 16 h. After completion (monitored by TLC), reaction mixture was diluted with water (30 mL) and solid was filtered. The solid was dried under reduced pressure to give the title compound (3, 180 mg crude) as a light-brown semi-solid. MS: m/z 571.30 (M+H)⁺

Step-2 Synthesis of N-(3-{[(3S)-3-aminopiperidin-1-yl]methyl}-5-(4-methyl-1H-imidazol-1-yl)phenyl)-4-(4-methyl-1H-imidazol-1-yl)pyridine-2-carboxamide (Compound 52): To a solution of 3 (100 mg, crude) in DCM (10 mL), 4M HCl in dioxane (10.0 mL) was added. After completion of reaction (monitored by TLC), the reaction mixture was concentrated in vacuo. The crude material was purified by prep-HPLC (reverse phase, Sunfire C18 (19×250) mm, 10 μm, gradient 10% to 90% ACN in water containing 5 mM Ammonium acetate, RT: 14.29 min to give the title compound (Compound 52, 10.0 mg, 10%) as a white solid.

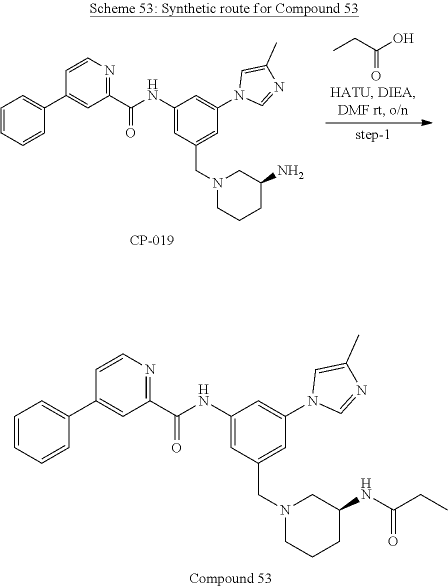

Step-1 The title compound Compound 53 (6.0 mg, 8.7% yield) was prepared according to the General Method E with CP-019 (75 mg, 0.13 mmol), propionic acid (12 mg, 0.16 mmol), HATU (60 mg, 0.16 mmol) and DIEA (34 mg, 0.26 mmol) in DMF (10 mL). LC/MS (ESI): m/z=523.3 [M+1]⁺ RT=6.44 min

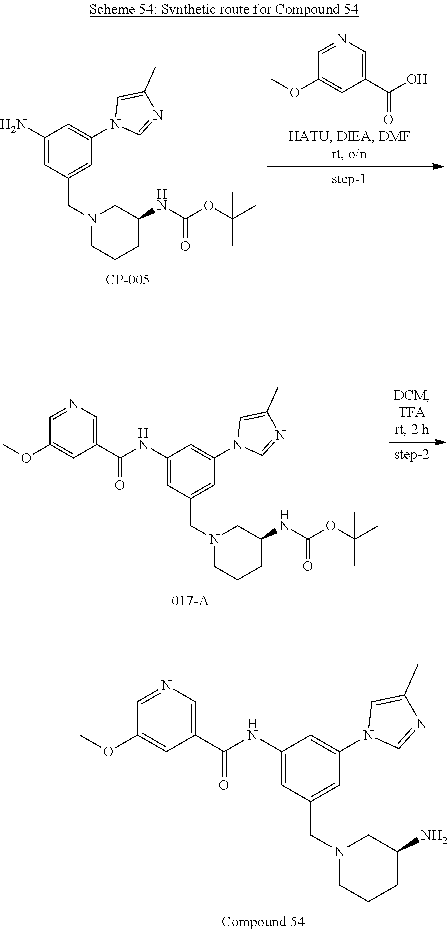

Step-1 The synthesis of 017-A: The title compound 017-A (150 mg, 0.29 mmol, 55% yield) was prepared according to the General Method E with CP-005 (200 mg, 0.52 mmol), 5-methoxynicotinic acid (120 mg, 0.78 mmol), HATU (300 mg, 0.78 mmol) and DIEA (270 mg, 2.08 mmol) in DMF (15 mL). LC/MS (ESI): m/z=521.4 [M+1]⁺ RT=2.10 min

Step-2 The synthesis of CP-017 Compound 54: The title compound Compound 54 (22 mg, 18% yield) was prepared according to the General Method G with 017-A (150 mg, 0.29 mmol) and TFA (5 mL) in DCM (15 mL).

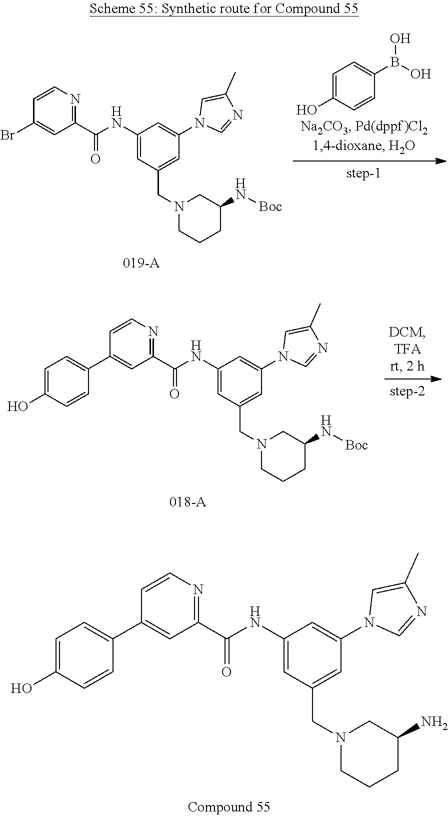 [SMILES: Cc1cn(-c2cc(CN3CCC[C@H](NC(=O)OC(C)(C)C)C3)cc(NC(=O)c3cc(Br)ccn3)c2)cn1 ; OB(O)c1ccc(O)cc1 ; Cc1cn(-c2cc(CN3CCC[C@H](NC(=O)OC(C)(C)C)C3)cc(NC(=O)c3cc(-c4ccc(O)cc4)ccn3)c2)cn1 ; Cc1cn(-c2cc(CN3CCC[C@H](N)C3)cc(NC(=O)c3cc(-c4ccc(O)cc4)ccn3)c2)cn1]

Step-1 The synthesis of 018-A: The title compound 018-A (120 mg, 0.21 mmol, 69% yield) was prepared according to the General Method F with 019-A (170 mg, 0.30 mmol), (4-hydroxyphenyl)boronic acid (62 mg, 0.45 mmol), Pd(dppf)Cl₂ (22 mg, 0.03 mmol) and K₂CO₃ (95 mg, 0.90 mmol) in 1, 4-dioxane (15 mL) and H₂O (3 mL). LC/MS (ESI): m/z=583.3 [M+1]⁺ RT=1.90 min

Step-2 The synthesis of Compound 55: The title compound Compound 55 (3.5 mg, 3.5% yield) was prepared according to the General Method G with 018-A (120 mg, 0.21 mmol) and TFA (5 mL) in DCM (15 mL).

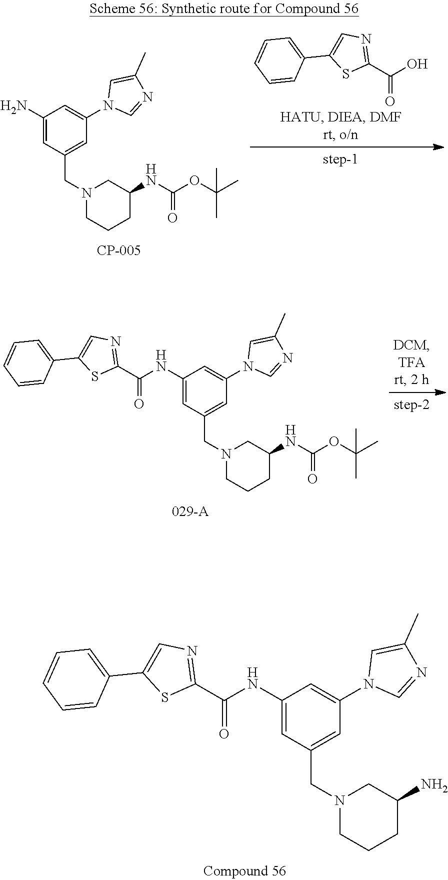 [SMILES: Cc1cn(-c2cc(N)cc(CN3CCC[C@H](NC(=O)OC(C)(C)C)C3)c2)cn1 ; O=C(O)c1ncc(-c2ccccc2)s1 ; Cc1cn(-c2cc(CN3CCC[C@H](NC(=O)OC(C)(C)C)C3)cc(NC(=O)c3ncc(-c4ccccc4)s3)c2)cn1 ; Cc1cn(-c2cc(CN3CCC[C@H](N)C3)cc(NC(=O)c3ncc(-c4ccccc4)s3)c2)cn1]

Step-1 The synthesis of 029-A: The title compound 029-A (120 mg, 0.21 mmol, 40% yield) was prepared according to the General Method E with CP-005 (200 mg, 0.52 mmol), 5-phenylthiazole-2-carboxylic acid (160 mg, 0.78 mmol), HATU (300 mg, 0.78 mmol) and DIEA (270 mg, 2.08 mmol) in DMF (15 mL). LC/MS (ESI): m/z=573.3 [M+1]⁺ RT=2.11 min

Step-2 The synthesis of Compound 56: The title compound Compound 56 (50 mg, 50% yield) was prepared according to the General Method G with 029-A (120 mg, 0.21 mmol) and TFA (5 mL) in DCM (15 mL).

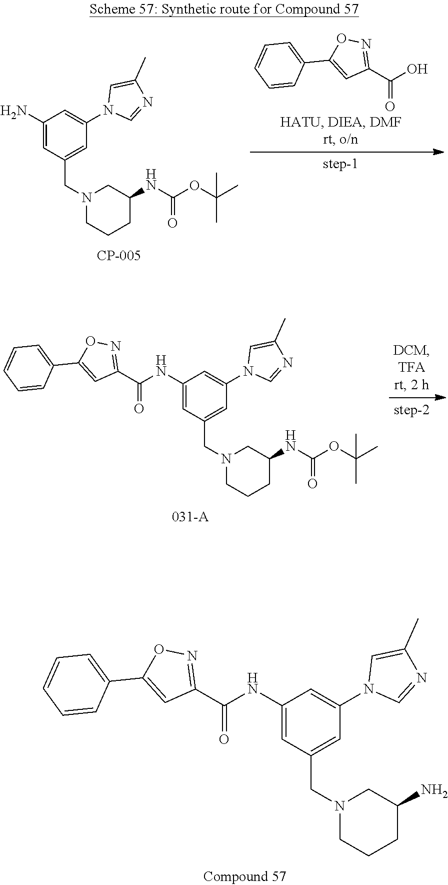 [SMILES: Cc1cn(-c2cc(N)cc(CN3CCC[C@H](NC(=O)OC(C)(C)C)C3)c2)cn1 ; O=C(O)c1cc(-c2ccccc2)on1 ; Cc1cn(-c2cc(CN3CCC[C@H](NC(=O)OC(C)(C)C)C3)cc(NC(=O)c3cc(-c4ccccc4)on3)c2)cn1 ; Cc1cn(-c2cc(CN3CCC[C@H](N)C3)cc(NC(=O)c3cc(-c4ccccc4)on3)c2)cn1]

Step-1 The synthesis of 031-A: The title compound 031-A (130 mg, 0.23 mmol, 45% yield) was prepared according to the General Method E with CP-005 (200 mg, 0.52 mmol), 5-phenylisoxazole-3-carboxylic acid (145 mg, 0.78 mmol), HATU (300 mg, 0.78 mmol) and DIEA (270 mg, 2.08 mmol) in DMF (15 mL). LC/MS (ESI): m/z=557.3 [M+1]⁺ RT=2.26 min

Step-2 The synthesis of Compound 57: The title compound Compound 57 (70 mg, 65% yield) was prepared according to the General Method G with 030-A (130 mg, 0.23 mmol) and TFA (5 mL) in DCM (15 mL).

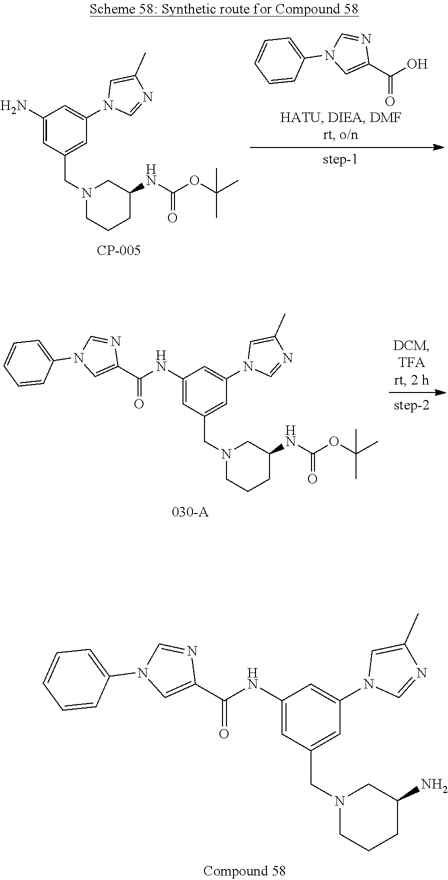 [SMILES: Cc1cn(-c2cc(N)cc(CN3CCC[C@H](NC(=O)OC(C)(C)C)C3)c2)cn1 ; O=C(O)c1cn(-c2ccccc2)cn1 ; Cc1cn(-c2cc(CN3CCC[C@H](NC(=O)OC(C)(C)C)C3)cc(NC(=O)c3cn(-c4ccccc4)cn3)c2)cn1 ; Cc1cn(-c2cc(CN3CCC[C@H](N)C3)cc(NC(=O)c3cn(-c4ccccc4)cn3)c2)cn1]

Step-1 The synthesis of 030-A: The title compound 030-A (135 mg, 0.24 mmol, 47% yield) was prepared according to the General Method E with CP-005 (200 mg, 0.52 mmol), 1-phenyl-1H-imidazole-4-carboxylic acid (145 mg, 0.78 mmol), HATU (300 mg, 0.78 mmol) and DIEA (270 mg, 2.08 mmol) in DMF (15 mL). LC/MS (ESI): m/z=556.3 [M+1]⁺ RT=2.08 min

Step-2 The synthesis of Compound 58: The title compound Compound 58 (50 mg, 45% yield) was prepared according to the General Method G with 030-A (135 mg, 0.24 mmol) and TFA (5 mL) in DCM (15 mL).

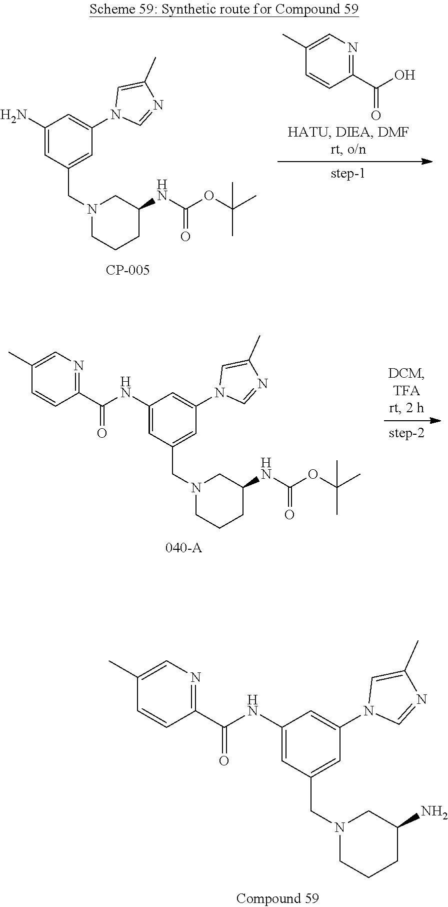

Step-1 The synthesis of 040-A: The title compound 040-A (160 mg, 0.32 mmol, 61% yield) was prepared according to the General Method E with CP-005 (200 mg, 0.52 mmol), 5-methylpicolinic acid (107 mg, 0.78 mmol), HATU (300 mg, 0.78 mmol) and DIEA (270 mg, 2.08 mmol) in DMF (15 mL). LC/MS (ESI): m/z=504.9 [M+1]⁺ RT=1.76 min

Step-2 The synthesis of Compound 59: The title compound Compound 59 (65 mg, 50.7% yield) was prepared according to the General Method G with 040-A (160 mg, 0.32 mmol) and TFA (5 mL) in DCM (15 mL).

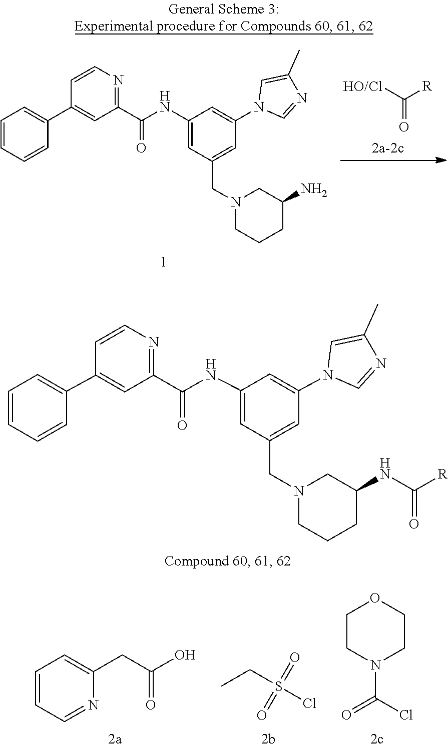

Structures of Compounds 60, 61 and 62:

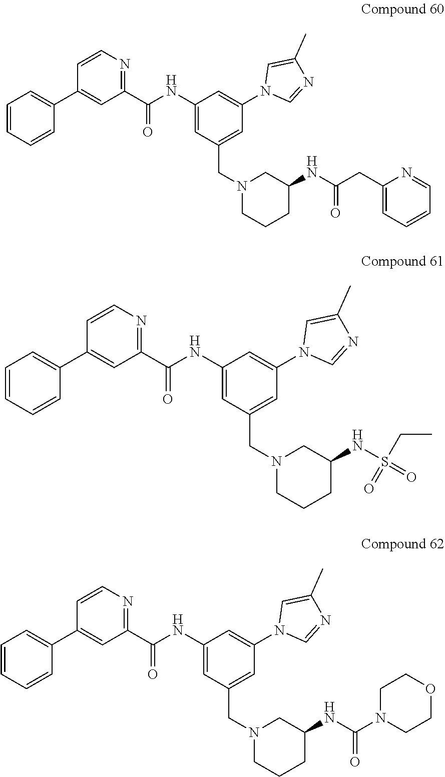 [SMILES: Cc1cn(-c2cc(CN3CCC[C@H](NC(=O)Cc4ccccn4)C3)cc(NC(=O)c3cc(-c4ccccc4)ccn3)c2)cn1 ; CCS(=O)(=O)N[C@H]1CCCN(Cc2cc(NC(=O)c3cc(-c4ccccc4)ccn3)cc(-n3cnc(C)c3)c2)C1 ; Cc1cn(-c2cc(CN3CCC[C@H](NC(=O)N4CCOCC4)C3)cc(NC(=O)c3cc(-c4ccccc4)ccn3)c2)cn1]

Experimental Procedure:

Synthesis of N-[3-(4-methyl-1H-imidazol-1-yl)-5-{[(3S)-3-[2-(pyridin-2-yl)acetamido]piperidin-1-yl]methyl}phenyl]-4-phenylpyridine-2-carboxamide (Compound 60): To the stirring solution of 1 (100 mg, 0.199 mmol) and 2-(pyridin-2-yl)acetic acid hydrochloride (2a, 34.5 mg, 0.199 mmol) in DCM (4.00 mL), ethylbis(propan-2-yl)amine (0.147 mL, 0.795 mmol) was added at 0° C. The reaction mixture was stirred at same temperature for 10 min and tripropyl-1,3,5,2λ⁵,4λ⁵,6λ⁵-trioxatriphosphinane-2,4,6-trione (253 mg, 0.398 mmol) was added at 0° C. The resulting mixture was stirred at room temperature for 16 h. After completion of reaction (monitored by TLC), the reaction mixture was quenched with sodium bicarbonate solution (30 mL) and the aqueous layer was extracted with DCM (2×50 mL). The combined organic layer was dried (sodium sulphate) and concentrated in vacuo. The crude was purified by flash column chromatography [normal phase, silica gel (100-200 mesh), gradient 0% to 5% MeOH in DCM] and triturated with ether and pentane (10 mL) to give the title compound (Compound 60, 42.0 mg, 36%) as an off-white solid.

Synthesis of N-(3-{[(3S)-3-ethanesulfonamidopiperidin-1-yl]methyl}-5-(4-methyl-1H-imidazol-1-yl)phenyl)-4-phenylpyridine-2-carboxamide (Compound 61): To a stirred solution of 1 (200 mg, 0.429 mmol) in DCM (10.0 mL) were added triethyl amine (86.8 mg, 0.857 mmol) and ethanesulfonyl chloride (2b, 0.093.1 mL, 1.07 mmol) at 0° C. and the reaction mixture was stirred at room temperature for 2 h. After completion of reaction (monitored by TLC), water was added in reaction mixture and extracted with DCM (2×20 mL), dried (sodium sulphate) and concentrated in vacuo. The crude was purified by flash column chromatography [normal phase, silica gel (100-200 mesh), gradient 0% to 5% MeOH in DCM] and triturated with ether and pentane (10 mL) to give the title compound (Compound 61, 20.0 mg, 8.35%) as a white solid.

Synthesis of N-[(3S)-1-{[3-(4-methyl-1H-imidazol-1-yl)-5-(4-phenylpyridine-2-amido)phenyl]methyl}piperidin-3-yl]morpholine-4-carboxamide (Compound 62): To a stirred solution of in 1 (100 mg, 214 μmol) in dichloromethane (10.0 mL) then, morpholine-4-carbonyl chloride was added and stirred the reaction mass at room temperature for 2 h. After completion of the reaction, the reaction was quenched with water (10 mL) and aqueous layer was extracted with DCM (2×30 mL), The organic layer were combined and dried with anhydrous Na₂SO₄ and concentrated in vacuo to give crude of material which was purified with Prep-HPLC to give the title compound (Compound 62, 24.2 mg, 18.7%) as a white solid.

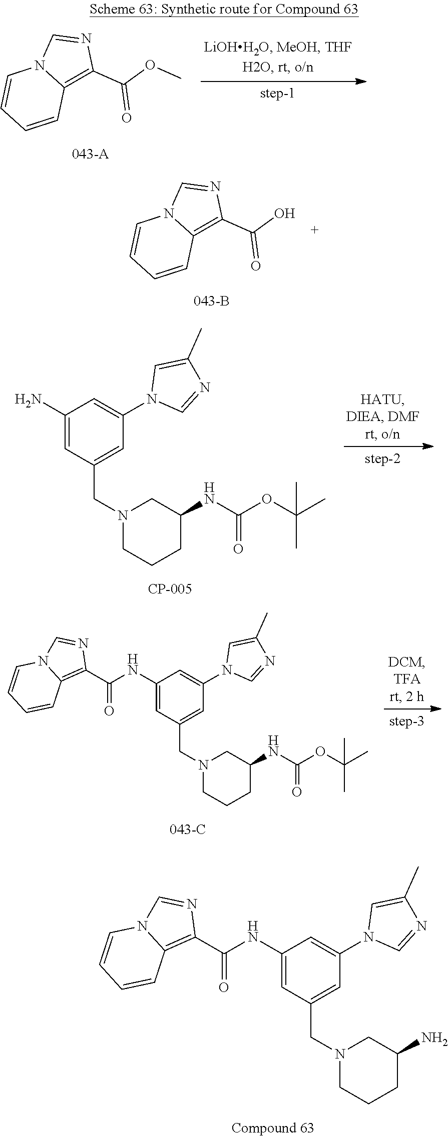 [SMILES: COC(=O)c1ncn2ccccc12 ; O=C(O)c1ncn2ccccc12 ; Cc1cn(-c2cc(N)cc(CN3CCC[C@H](NC(=O)OC(C)(C)C)C3)c2)cn1 ; Cc1cn(-c2cc(CN3CCC[C@H](NC(=O)OC(C)(C)C)C3)cc(NC(=O)c3ncn4ccccc34)c2)cn1 ; Cc1cn(-c2cc(CN3CCC[C@H](N)C3)cc(NC(=O)c3ncn4ccccc34)c2)cn1]

Step-1 The synthesis of 043-B: General Method H: To a solution of methyl imidazo[1,5-a]pyridine-1-carboxylate (200 mg, 1.14 mmol) in THF (10 mL), MeOH (10 mL) and H₂O (10 mL) was added LiOH·H₂O (630 mg, 15.0 mmol), and the reaction mixture was stirred at room temperature for 2 h. After consumption of the starting material, the volatiles were evaporated under reduced pressure. The resulted mixture was diluted by water (10 mL) and acidified by 2N HCl to give a solid. The solid was collected, washed by water and dried to give 043-B (165 mg, 90% yield) as a white solid. LC/MS (ESI): m/z=163.0 [M+1]⁺ RT=0.24 min

Step-2 The synthesis of 043-C: The title compound 043-C (160 mg, 0.30 mmol, 58% yield) was prepared according to the General Method E with CP-005 (200 mg, 0.52 mmol), 043-B (85 mg, 0.52 mmol), HATU (257 mg, 0.68 mmol) and DIEA (200 mg, 1.56 mmol) in DMF (15 mL). LC/MS (ESI): m/z=529.9 [M+1]⁺ RT=1.64 min

Step-3 The synthesis of Compound 63: The title compound Compound 63 (65 mg, 50% yield) was prepared according to the General Method G with 043-C (160 mg, 0.30 mmol) and TFA (5 mL) in DCM (15 mL).

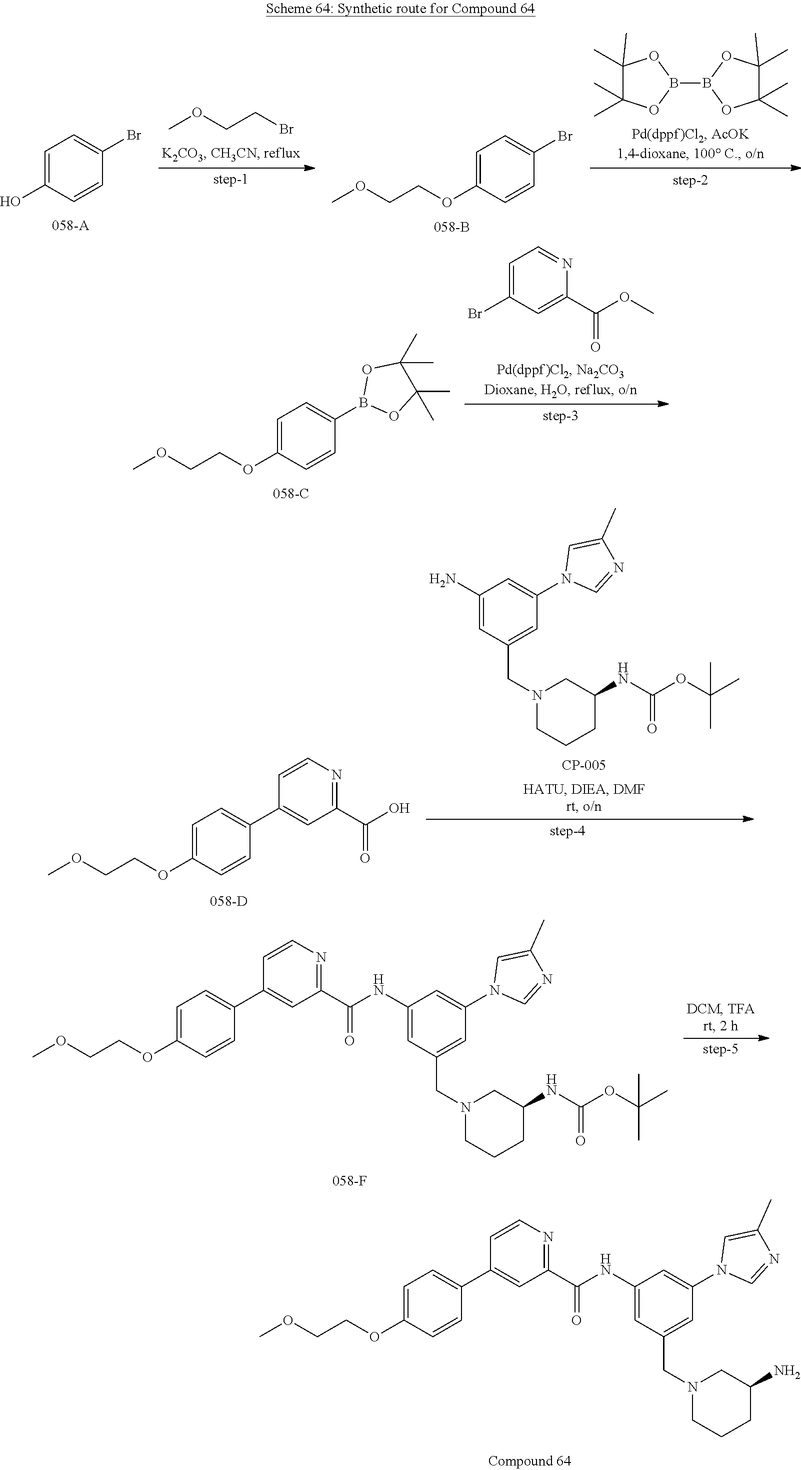

Step-1 The synthesis of 058-B: General Method B2: To a mixture of 1-bromo-2-methoxyethane (1.67 g, 12 mmol), 4-bromophenol (1.73, 10 mmol) in CH₃CN (50 mL) was added K₂CO₃ (2.08 g, 20 mmol), and the reaction mixture was stirred at 85° C. overnight. After consumption of the starting material, water (300 mL) was added and the mixture was extracted with EtOAc (3×100 mL). The combined organic layers were washed with brine (300 mL), dried over anhydrous Na₂SO₄ and concentrated under reduced pressure to give a residue. The residue was purified by column chromatography on silica gel (eluting with 15% EA in PE) to give 058-B (1.68 g, 73% yield) as a yellow solid. LC/MS (ESI): no mass signal. RT=1.65 min

Step-2 The synthesis of 058-C: General Method I: To a mixture of 058-B (1.36 g, 4 mmol), Bis(pinacolato)diborane (2.24 g, 6 mmol), and KOAc (1.15 g, 8 mmol) in 1, 4-dioxane (50 mL) was added Pd(dppf)Cl₂ (429 mg, 0.4 mmol), and the reaction mixture was purged with Ar, then stirred at 80° C. overnight. After cooling to room temperature, it was diluted by water (200 mL) and extracted with EtOAc (2×100 mL). The organic extract was washed by brine (200 mL), dried over anhydrous Na₂SO₄ and evaporated under reduced pressure to give a residue. The residue was purified by column chromatography on silica gel (eluting with 20% EA in PE) to give 058-C (1.3 g, 78% yield) as grey solid. LC/MS (ESI): m/z=296 [M+18]⁺. RT=1.78 min

Step-3 The synthesis of 058-D: The title compound 058-D (655 mg, 2.43 mmol, 80% yield) was prepared according to the General Method F with 058-C (1.25 g, 4.5 mmol), methyl 4-bromopicolinate (668 mg, 3 mmol), Na₂CO₃ (636 mg, 6 mmol) and Pd(dppf)Cl₂ (220 mg, 0.3 mmol) in 1,4-dioxane (30 mL) and H₂O (5 mL). LC/MS (ESI): m/z=274.0 [M+1]⁺. RT=0.99 min

Step-4 The synthesis of 058-F: The title compound 058-F (176 mg, 0.27 mmol, 53% yield) was prepared according to the General Method E with 058-D (156 mg, 0.57 mmol), CP-005 (200 mg, 0.52 mmol), HATU (296 mg, 0.78 mmol) and DIEA (202 mg, 1.56 mmol) in DMF (30 mL). LC/MS (ESI): m/z=640.8 [M+1]⁺. RT=1.88 min

Step-5 The synthesis of Compound 64: The title compound Compound 64 (21 mg, 14% yield) was prepared according to the General Method G with 058-C (176 mg, 0.27 mmol) and TFA (3 mL) in DCM (10 mL).

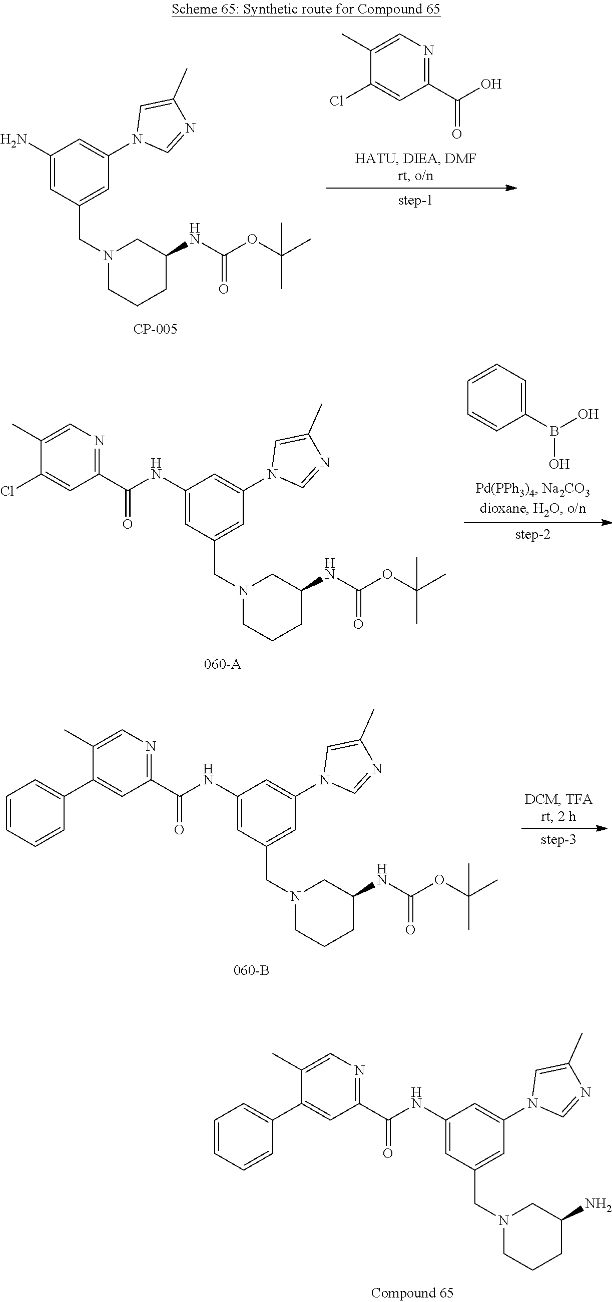 [SMILES: Cc1cn(-c2cc(N)cc(CN3CCC[C@H](NC(=O)OC(C)(C)C)C3)c2)cn1 ; Cc1cnc(C(=O)O)cc1Cl ; Cc1cn(-c2cc(CN3CCC[C@H](NC(=O)OC(C)(C)C)C3)cc(NC(=O)c3cc(Cl)c(C)cn3)c2)cn1 ; OB(O)c1ccccc1 ; Cc1cn(-c2cc(CN3CCC[C@H](NC(=O)OC(C)(C)C)C3)cc(NC(=O)c3cc(-c4ccccc4)c(C)cn3)c2)cn1 ; Cc1cn(-c2cc(CN3CCC[C@H](N)C3)cc(NC(=O)c3cc(-c4ccccc4)c(C)cn3)c2)cn1]

Step-1 The synthesis of 060-A: The title compound 060-A (240 mg, 0.45 mmol, 68.7% yield) was prepared according to the General Method E with CP-005 (250 mg, 0.65 mmol), 4-chloro-5-methylpicolinic acid (111 mg, 0.65 mmol), HATU (320 mg, 0.84 mmol) and DIEA (250 mg, 1.95 mmol) in DMF (15 mL). LC/MS (ESI): m/z=539.1 [M+1]⁺. RT=0.60 min

Step-2 The synthesis of 060-B: The title compound 060-B (180 mg, 0.31 mmol, 69% yield) was prepared according to the General Method F with 060-A (240 mg, 0.45 mmol), phenylboronic acid (80 mg, 0.67 mmol), Pd(dppf)Cl₂ (32 mg, 0.045 mmol) and K₂CO₃ (185 mg, 1.34 mmol) in 1, 4-dioxane (20 mL) and H₂O (4 mL). LC/MS (ESI): m/z=580.8 [M+1]⁺. RT=1.99 min

Step-3 The synthesis of Compound 65: The title compound Compound 65 (16 mg, 10.7% yield) was prepared according to the General Method G with 060-B (180 mg, 0.31 mmol) and TFA (3 mL) in DCM (10 mL).

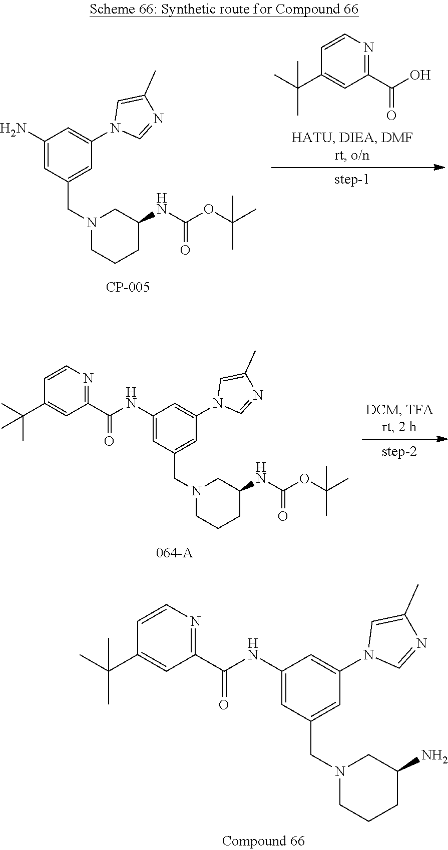

Step-1 The synthesis of 064-A: The title compound 064-A (145 mg, 0.26 mmol, 51% yield) was prepared according to the General Method E with CP-005 (200 mg, 0.52 mmol), 4-(tert-butyl)picolinic acid (140 mg, 0.78 mmol), HATU (300 mg, 0.78 mmol) and DIEA (270 mg, 2.08 mmol) in DMF (15 mL). LC/MS (ESI): m/z=547.0 [M+1]⁺. RT=2.39 min

Step-2 The synthesis of Compound 66: The title compound Compound 66 (60 mg, 50.7% yield) was prepared according to the General Method G with 064-A (145 mg, 0.26 mmol) and TFA (5 mL) in DCM (15 mL).

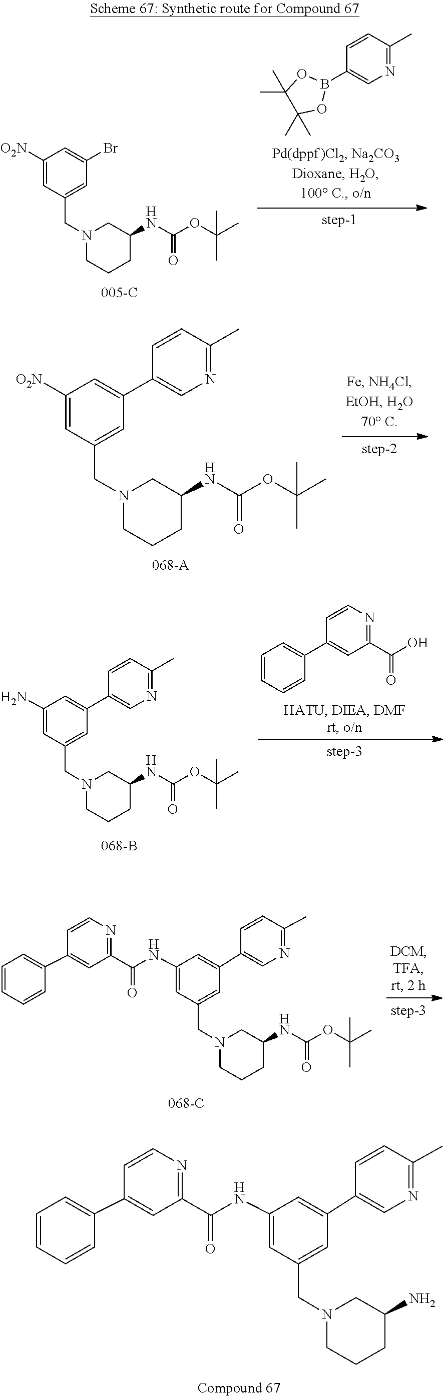

Step-1 The synthesis of 068-A: The title compound 068-A (350 mg, 0.82 mmol, 84% yield) was prepared according to the General Method F with 005-C (414 mg, 1 mmol), 2-methyl-5-(4,4,5,5-tetramethyl-1,3,2-dioxaborolan-2-yl)pyridine (328 mg, 1.5 mmol), Pd(dppf)Cl₂ (73 mg, 0.1 mmol) and K₂CO₃ (276 mg, 2 mmol) in 1, 4-dioxane (25 mL) and H₂O (5 mL). LC/MS (ESI): m/z=427.0 [M+1]⁺. RT=1.81 min

Step-2 The synthesis of 068-B: To a solution of 068-A (340 mg, 0.8 mmol), NH₄Cl (256 mg, 4.8 mmol) in EtOH (30 mL) and H₂O (6 mL) was added Fe powder (403 mg, 7.2 mmol), and the reaction mixture was stirred at room temperature for 3 hours.

After consumption of the starting material, the reaction mixture was filtered through a celite pad to remove the solid and the filtrate was diluted by H₂O (3 mL) and extracted with EtOAc (2×30 mL). The organic extract was washed by brine (30 mL), dried over Na₂SO₄, evaporated under reduced pressure to give crude 068-B (310 mg, 95% yield) as black solid. LC/MS (ESI): m/z=397.0 [M+1]⁺. RT=1.57 min

Step-3 The synthesis of 068-C: The title compound 068-C (102 mg, 0.176 mmol, 35% yield) was prepared according to the General Method E with 068-B (200 mg, 0.5 mmol), 4-phenylpicolinic acid (100 mg, 0.5 mmol), HATU (285 mg, 0.75 mmol) and DIEA (193 mg, 1.5 mmol) in DMF (15 mL). LC/MS (ESI): m/z=577.9 [M+1]⁺. RT=2.49 min

Step-4 The synthesis of Compound 67: The title compound Compound 67 (37 mg mmol, 45% yield) was prepared according to the General Method G with 068-C (102 mg, 0.17 mmol) and TFA (3 mL) in DCM (10 mL).

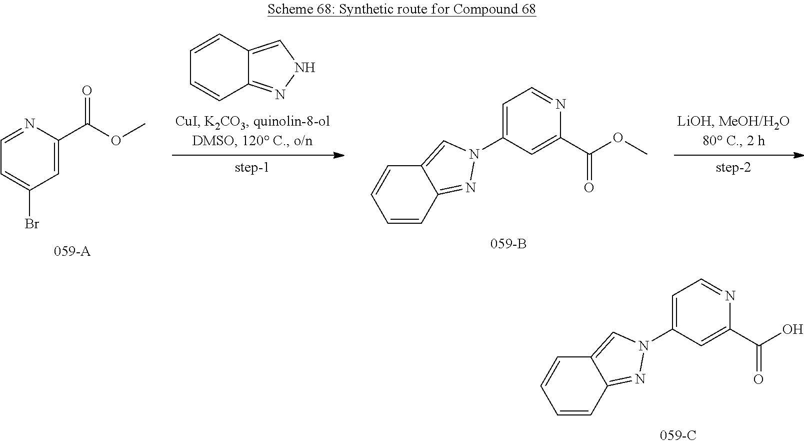 [SMILES: COC(=O)c1cc(Br)ccn1 ; c1ccc2n[nH]cc2c1 ; COC(=O)c1cc(-n2cc3ccccc3n2)ccn1 ; O=C(O)c1cc(-n2cc3ccccc3n2)ccn1]

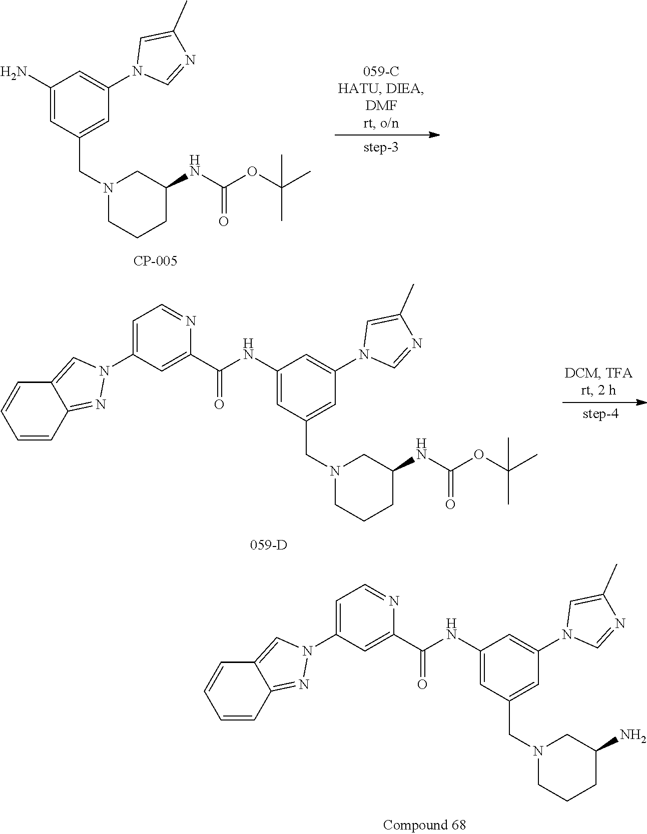 [SMILES: Cc1cn(-c2cc(N)cc(CN3CCC[C@H](NC(=O)OC(C)(C)C)C3)c2)cn1 ; Cc1cn(-c2cc(CN3CCC[C@H](NC(=O)OC(C)(C)C)C3)cc(NC(=O)c3cc(-n4cc5ccccc5n4)ccn3)c2)cn1 ; Cc1cn(-c2cc(CN3CCC[C@H](N)C3)cc(NC(=O)c3cc(-n4cc5ccccc5n4)ccn3)c2)cn1]

Step-1 The synthesis of 059-B: The title compound 059-B (135 mg, 0.53 mmol, 23% yield) was prepared according to the General Method C with 059-A (500 mg, 2.33 mmol), 2H-indazole (823 mg, 6.99 mmol), K₂CO₃ (642 mg, 4.66 mmol), CuI (133 mg, 0.70 mmol) and quinolin-8-ol (67 mg, 0.47 mmol) in DMSO (15 mL).

Step-2 The synthesis of 059-C: The title compound 059-C (120 mg, 0.50 mmol, 95% yield) was prepared according to the General Method E with methyl 4-(2H-indazol-2-yl)picolinate (059-B, 135 mg, 0.53 mmol) in MeOH (10 mL) and H₂O (10 mL) was added LiOH·H₂O (630 mg, 15.0 mmol), and the reaction mixture was stirred at 80° C. for 2 h. LC/MS (ESI): m/z=240.2 [M+1]⁺. RT=0.43 min

Step-3 The synthesis of 059-D: The title compound 059-D (120 mg, 0.20 mmol, 38% yield) was prepared according to the General Method E with CP-005 (200 mg, 0.52 mmol), 059-E (186 mg, 0.78 mmol), HATU (300 mg, 0.78 mmol) and DIEA (270 mg, 2.08 mmol) in DMF (15 mL). LC/MS (ESI): m/z=607.3 [M+1]⁺. RT=2.07 min

Step-4 The synthesis of Compound 68: The title compound Compound 68 (42 mg, 42% yield) was prepared according to the General Method G with 059-D (120 mg, 0.20 mmol) and TFA (3 mL) in DCM (10 mL).

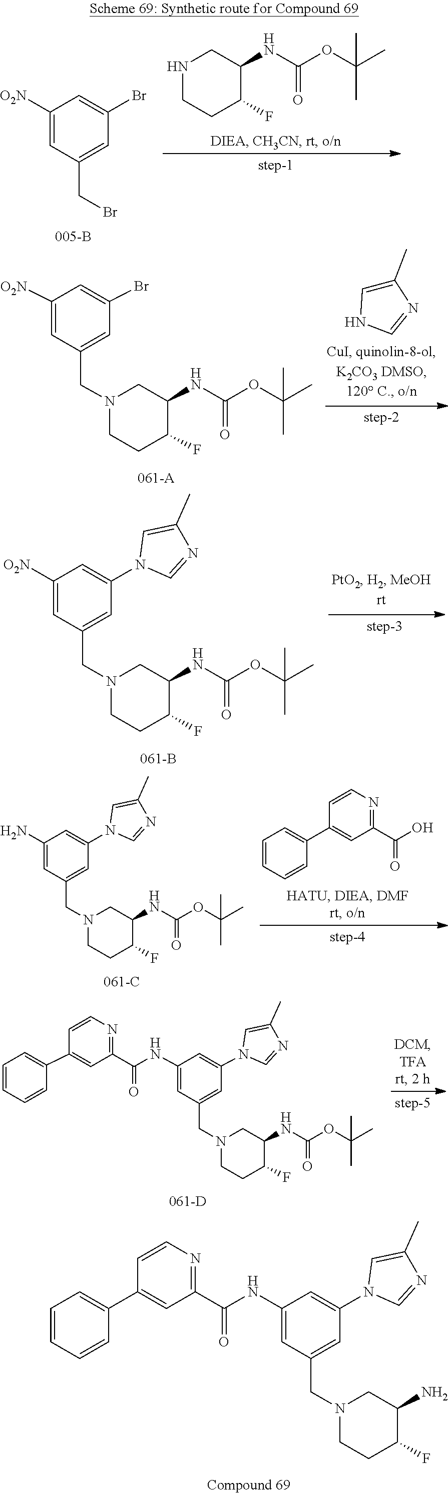

Step-1 The synthesis of 061-A: The title compound 061-A (420 mg, 0.97 mmol, 97% yield) was prepared according to the General Method B1 with 005-B (367 mg, 1 mmol), tert-butyl ((3R,4R)-4-fluoropiperidin-3-yl)carbamate (218 mg, 1 mmol) and DIEA (193 mg, 1.5 mmol) in CH₃CN (30 mL). LC/MS (ESI): m/z=433.8 [M+1]⁺. RT=1.91 min

Step-2 The synthesis of 061-B: to a mixture of 061-A (656 mg, 1.5 mmol), 4-methyl-1H-imidazole (370 mg, 4.5 mmol), CuI (85.5 mg, 0.45 mmol), and K₂CO₃ (414 mg, 3 mmol) in DMSO (30 mL) was added quinolin-8-ol (44 mg, 0.3 mmol), then the reaction mixture was purged with Ar, and stirred at 120° C. overnight. After cooling to room temperature, it was diluted by water (100 mL) and extracted with EtOAc (2×50 mL). The organic extract was washed by H₂O (4×100 mL) and brine (100 mL), dried over anhydrous Na₂SO₄ and evaporated under reduced pressure to give a residue. The residue was purified by column chromatography on silica gel (eluting with 3% MeOH in DCM) to give 061-B (202 mg, 66% yield) as a yellow solid. LC/MS (ESI): m/z=434.0 [M+1]⁺. RT=2.04 min

Step-3 The synthesis of 061-C: The title compound 061-C (180 mg, 0.45 mmol, 90% yield) was prepared according to the General Method D1 with 061-B (220 mg, 0.5 mmol), PtO₂ (40 mg, 0.18 mmol) in MeOH (30 mL). LC/MS (ESI): m/z=404.0 [M+1]⁺. RT=1.82 min

Step-4 The synthesis of 061-D: The title compound 061-D (100 mg, 0.17 mmol, 38% yield) was prepared according to the General Method E with 061-C (180 mg, 0.45 mmol), 4-phenylpicolinic acid (100 mg, 0.45 mmol), HATU (285 mg, 0.7 mmol) and DIEA (193 mg, 1.35 mmol) in DMF (30 mL). LC/MS (ESI): m/z=584.9 [M+1]⁺. RT=2.32 min

Step-5 The synthesis of Compound 69: The title compound Compound 69 (22 mg, 26% yield) was prepared according to the General Method G with 061-D (100 mg, 0.17 mmol) and TFA (3 mL) in DCM (10 mL).

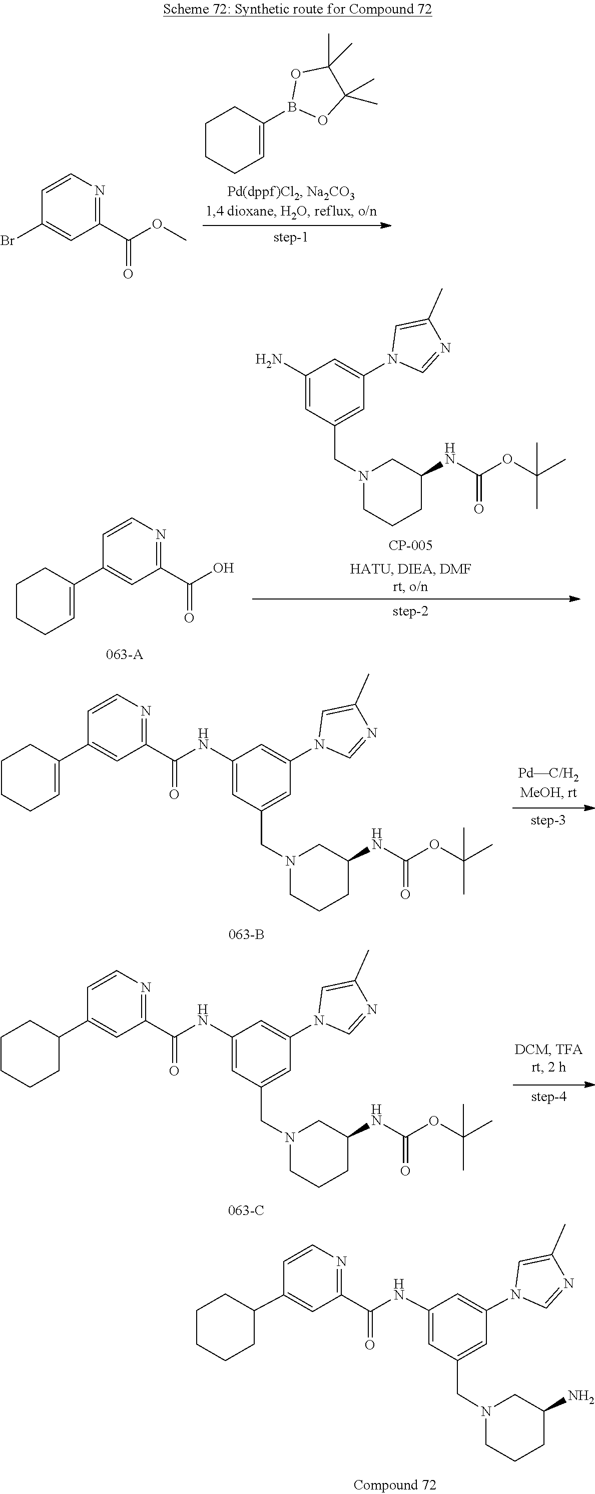

Step-1 The synthesis of 063-A: The title compound 063-A (250 mg, 1.23 mmol, 40% yield) was prepared according to the General Method F with methyl 4-bromopicolinate (668 mg, 3 mmol), 2-(cyclohex-1-en-1-yl)-4,4,5,5-tetramethyl-1,3,2-dioxaborolane (936 mg, 4.5 mmol), Pd(dppf)Cl₂ (220 mg, 0.3 mmol) and K₂CO₃ (636 mg, 6 mmol) in 1, 4-dioxane (30 mL) and H₂O (6 mL). LC/MS (ESI): m/z=204.0 [M+1]⁺. RT=1.05 min

Step-2 The synthesis of 063-B: The title compound 063-B (230 mg, 0.40 mmol, 50% yield) was prepared according to the General Method E with 063-A (162 mg, 0.80 mmol), CP-005 (308 mg, 0.80 mmol), HATU (465 mg, 1.2 mmol) and DIEA (309 mg, 2.4 mmol) in DMF (30 mL). LC/MS (ESI): m/z=571.0 [M+1]⁺. RT=2.56 min

Step-3 The synthesis of 063-C: To a solution of 063-B (230 mg, 0.4 mmol) in MeOH (30 mL) was added 10% Pd/C (100 mg), and the reaction mixture was stirred at room temperature for 1.5 days under hydrogen atmosphere. After consumption of the starting material, the reaction mixture was filtered through a celite pad to remove the solid and the filtrate was evaporated under reduced pressure to give crude 063-C (177 mg, 78% yield) as a black solid. LC/MS (ESI): m/z=573.0 [M+1]⁺. RT=2.54 min

Step-4 The synthesis of Compound 72: The title compound Compound 72 (11 mg, 0.02 mmol, 8% yield) was prepared according to the General Method G with 063-C (177 mg, 0.3 mmol) and TFA (3 mL) in DCM (10 mL).

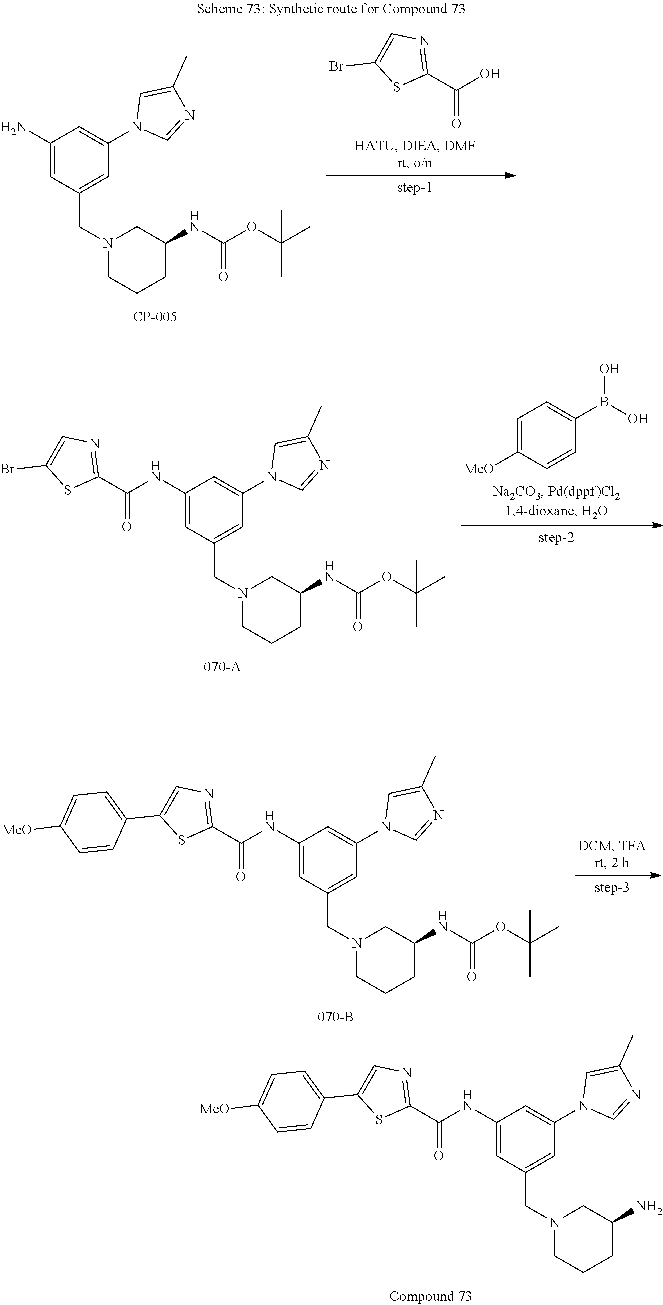 [SMILES: Cc1cn(-c2cc(N)cc(CN3CCC[C@H](NC(=O)OC(C)(C)C)C3)c2)cn1 ; O=C(O)c1ncc(Br)s1 ; Cc1cn(-c2cc(CN3CCC[C@H](NC(=O)OC(C)(C)C)C3)cc(NC(=O)c3ncc(Br)s3)c2)cn1 ; COc1ccc(B(O)O)cc1 ; COc1ccc(-c2cnc(C(=O)Nc3cc(CN4CCC[C@H](NC(=O)OC(C)(C)C)C4)cc(-n4cnc(C)c4)c3)s2)cc1 ; COc1ccc(-c2cnc(C(=O)Nc3cc(CN4CCC[C@H](N)C4)cc(-n4cnc(C)c4)c3)s2)cc1]

Step-1 The synthesis of 070-A: The title compound 070-A (650 mg, 1.13 mmol, 48% yield) was prepared according to the General Method E with CP-005 (900 mg, 2.34 mmol), 5-bromothiazole-2-carboxylic acid (726 mg, 3.51 mmol), HATU (1.33 g, 3.51 mmol) and DIEA (1.21 g, 9.36 mmol) in DMF (15 mL). LC/MS (ESI): m/z=575.0 [M+1]⁺. RT=1.99 min

Step-2 The synthesis of 070-B: The title compound 070-B (150 mg, 0.25 mmol, 71% yield) was prepared according to the General Method F with 070-A (200 mg, 0.35 mmol), (4-methoxyphenyl)boronic acid (80 mg, 0.53 mmol), Pd(dppf)Cl₂ (26 mg, 0.035 mmol) and Na₂CO₃ (111 mg, 1.05 mmol) in 1, 4-dioxane (15 mL) and H₂O (3 mL). LC/MS (ESI): m/z=603.0 [M+1]⁺. RT=1.91 min

Step-3 The synthesis of Compound 73: The title compound Compound 73 (60 mg, 48% yield) was prepared according to the General Method G with 070-B (150 mg, 0.25 mmol) and TFA (5 mL) in DCM (15 mL).

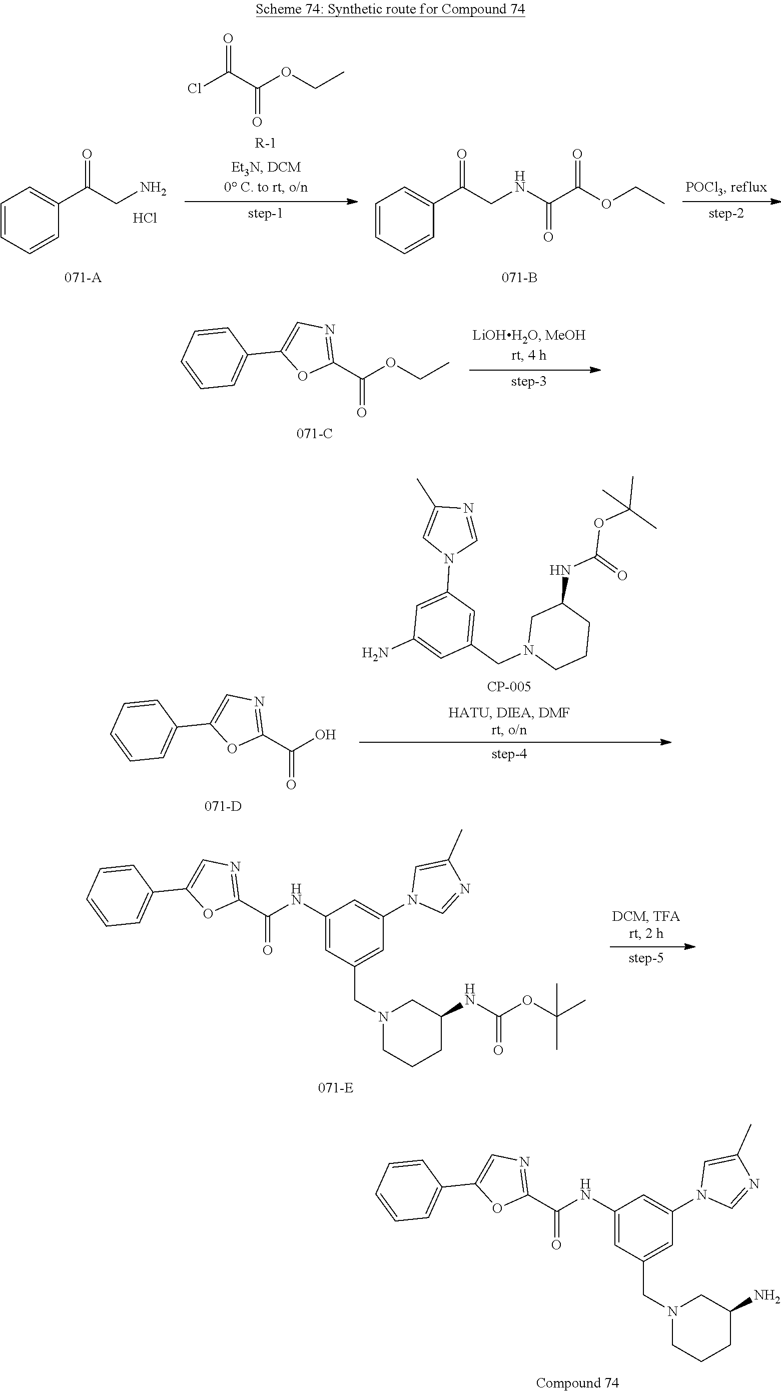

Step-1 The synthesis of 071-B: General Method K: To a mixture of 2-amino-1-phenylethanone hydrochloride (071-A, 3.68 g, 21.42 mmol) and ethyl 2-chloro-2-oxoacetate (R-1, 3.07 g, 22.49 mmol) in DCM (75 mL) was added Et₃N (4.55 g, 45.0 mmol) slowly, and the reaction mixture was allowed to stir at room temperature overnight. After consumption of the starting material, the reaction mixture was concentrated, then diluted by water (70 mL) and extracted with DCM (2×70 mL). The organic extract was washed by 2N HCl (100 mL) and brine (100 mL) successively, dried over anhydrous Na₂SO₄, filtered and evaporated under reduced pressure to give crude 071-B (3.49 g, 69% yield) as brown gel. LC/MS (ESI): m/z=236.2 [M+1]⁺. RT=0.60 min

Step-2 The synthesis of 071-C: A mixture of 071-B (3.49 g, 14.84 mmol) and POCl₃ (30 mL) was heated to reflux and stirred at the same temperature overnight. After consumption of the starting material, the reaction mixture was concentrated to remove excess POCl₃, then poured into crushed ice and extracted with EtOAc (2×70 mL). The organic extract was washed by aqueous Na₂CO₃ (100 mL) and brine (100 mL) successively, dried over anhydrous Na₂SO₄, filtered and evaporated under reduced pressure to give a residue. The residue was purified by column chromatography on silica gel (eluting with 10˜15% EA in PE) to give 071-C (2.19 g, 68% yield) as brown solid. LC/MS (ESI): m/z=218.2 [M+1]⁺. RT=0.73 min

Step-3 The synthesis of 071-D: The title compound 071-D (340 mg, 1.80 mmol, 90% yield) was prepared according to the General Method H with 071-C (434 mg, 2.0 mmol) and LiOH·H₂O (430 mg, 10.0 mmol) in THF (10 mL), MeOH (10 mL) and H₂O (10 mL). LC/MS (ESI): m/z=190.2 [M+1]⁺. RT=0.55 min

Step-4 The synthesis of 071-E: The title compound 071-E (120 mg, 0.21 mmol, 41% yield) was prepared according to the General Method E with CP-005 (200 mg, 0.52 mmol), 071-D (128 mg, 0.68 mmol), HATU (260 mg, 0.68 mmol) and DIEA (200 mg, 1.56 mmol) in DMF (15 mL). LC/MS (ESI): m/z=557.3 [M+1]⁺. RT=2.17 min

Step-5 The synthesis of Compound 74: The title compound Compound 74 (39 mg, 40% yield) was prepared according to the General Method G with 071-E (120 mg, 0.22 mmol) and TFA (5 mL) in DCM (15 mL).

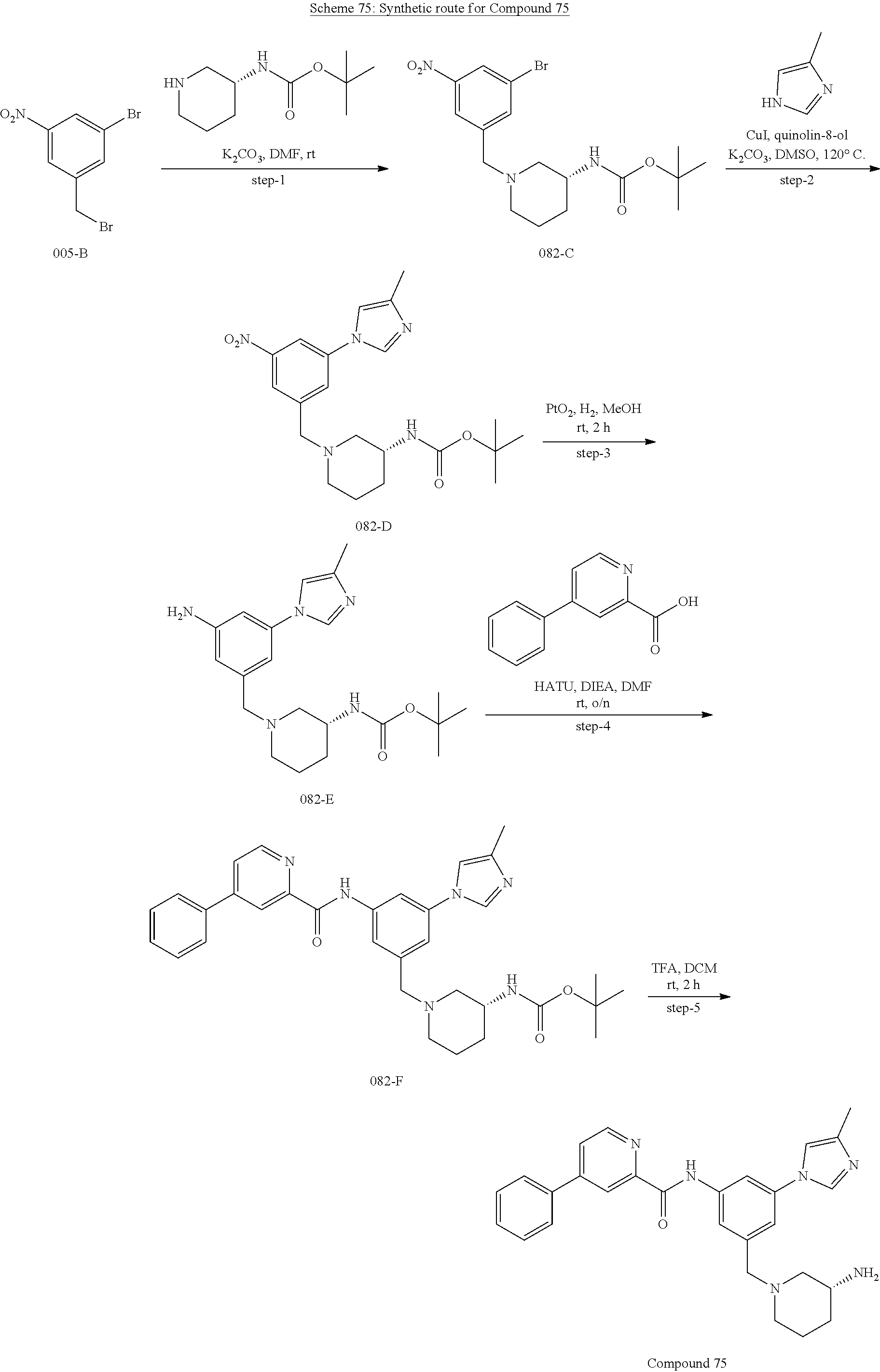

Step-1 The synthesis of 082-C: To a mixture of 005-B (1.0 g, 3.41 mmol), (R)-tert-butyl piperidin-3-ylcarbamate (680 mg, 3.41 mmol) in DMF (30 mL) was added K₂CO₃ (880 mg, 6.82 mmol), and the reaction mixture was stirred at room temperature overnight. After consumption of the starting material, water (50 mL) was added and the mixture was extracted with EtOAc (2×50 mL). The combined organic layers were washed with water (4×50 mL) and brine (50 mL) successively, dried over anhydrous Na₂SO₄ and concentrated under reduced pressure to give a residue. The residue was purified by column chromatography on silica gel (eluting with 20% EA in PE) to give 082-C (800 mg, 57% yield) as a yellow solid. LC/MS (ESI): m/z=414.0 [M+1]⁺. RT=2.16 min

Step-2 The synthesis of 082-D: The title compound 082-D (710 mg, 1.71 mmol, 88% yield) was prepared according to the General Method C with 082-C (800 mg, 1.94 mmol), 4-methyl-1H-imidazole (477 mg, 5.82 mmol), K₂CO₃ (535 mg, 3.88 mmol), CuI (110 mg, 0.58 mmol) and quinolin-8-ol (56 mg, 0.39 mmol) in DMSO (30 mL). LC/MS (ESI): m/z=416.0 [M+1]⁺. RT=1.72 min

Step-3 The synthesis of 082-E: The title compound 082-E (200 mg, 0.52 mmol, 30% yield) was prepared according to the General Method D1 with 082-D (710 mg, 1.71 mmol) and PtO₂ (70 mg) in MeOH (20 mL). LC/MS (ESI): m/z=386.0 [M+1]⁺. RT=1.51 min

Step-4 The synthesis of 082-F: The title compound 082-F (90 mg, 0.16 mmol, 30% yield) was prepared according to the General Method E with 082-E (200 mg, 0.52 mmol), 4-phenylpicolinic acid (114 mg, 0.57 mmol), HATU (296 mg, 0.78 mmol) and DIEA (268 mg, 2.08 mmol) in DMF (15 mL). LC/MS (ESI): m/z=567.0 [M+1]⁺. RT=2.13 min

Step-5 The synthesis of Compound 75: The title compound Compound 75 (30 mg, 40% yield) was prepared according to the General Method G with 082-F (90 mg, 0.16 mmol) and TFA (3 mL) in DCM (10 mL).

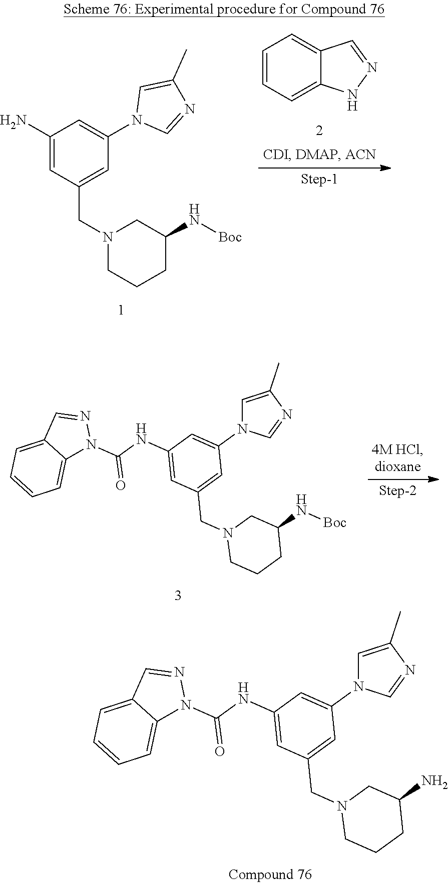 [SMILES: Cc1cn(-c2cc(N)cc(CN3CCC[C@H](NC(=O)OC(C)(C)C)C3)c2)cn1 ; c1ccc2[nH]ncc2c1 ; Cc1cn(-c2cc(CN3CCC[C@H](NC(=O)OC(C)(C)C)C3)cc(NC(=O)n3ncc4ccccc43)c2)cn1 ; Cc1cn(-c2cc(CN3CCC[C@H](N)C3)cc(NC(=O)n3ncc4ccccc43)c2)cn1]

Step-1 Synthesis of tert-butyl (S)-(1-(3-(1H-indazole-1-carboxamido)-5-(4-methyl-1H-imidazol-1-yl)benzyl)piperidin-3-yl)carbamate (3): To a solution of 1H-indazole (2, 100 mg, 0.846 mmol) in CH₃CN (5 mL), CDI (165 mg, 1.02 mmol) was added followed by addition of DMAP (catalytic) and stirred the reaction mixture at 80° C. for 16 h. Then the reaction was cooled to room temperature, amine 1 (163 mg, 0.42 mmol) was added and heated at 80° C. for 16 h. After completion of reaction (monitored by TLC), water (10 mL) was added and compound was extracted with DCM (3×50 mL). The organic layer was dried (Na₂SO₄) and concentrated in vacuo. The residue was purified by flash column chromatography [normal phase, silica gel (100-200 mesh), gradient 5% to 10% methanol in DCM] to give the title compound 3 (60 mg, 13%) as an off-white solid. LCMS: m/z 574.20 (M+H)⁺ (ES+), 88.79%

Step-2 Synthesis of N-(3-{[(3S)-3-aminopiperidin-1-yl]methyl}-5-(4-methyl-1H-imidazol-1-yl)phenyl)-1H-indazole-1-carboxamide (Compound 76): To a solution of 3 (60 mg, 0.113 mmol) in DCM (2 mL), 4N HCl in dioxane (2 mL) was added and stirred the reaction mass at room temperature for 4 h. After completion of reaction, the crude was concentrated in vacuo and the reaction mass was neutralized with saturated NaHCO₃ solution (20 mL) and compound was extracted with 5% MeOH/DCM (3×40 mL). The organic layer was dried (Na₂SO₄) and concentrated in vacuo and this was triturated with diethyl ether/pentane to give the title compound (Compound 76, 28 mg, 57%) as an off-white solid.

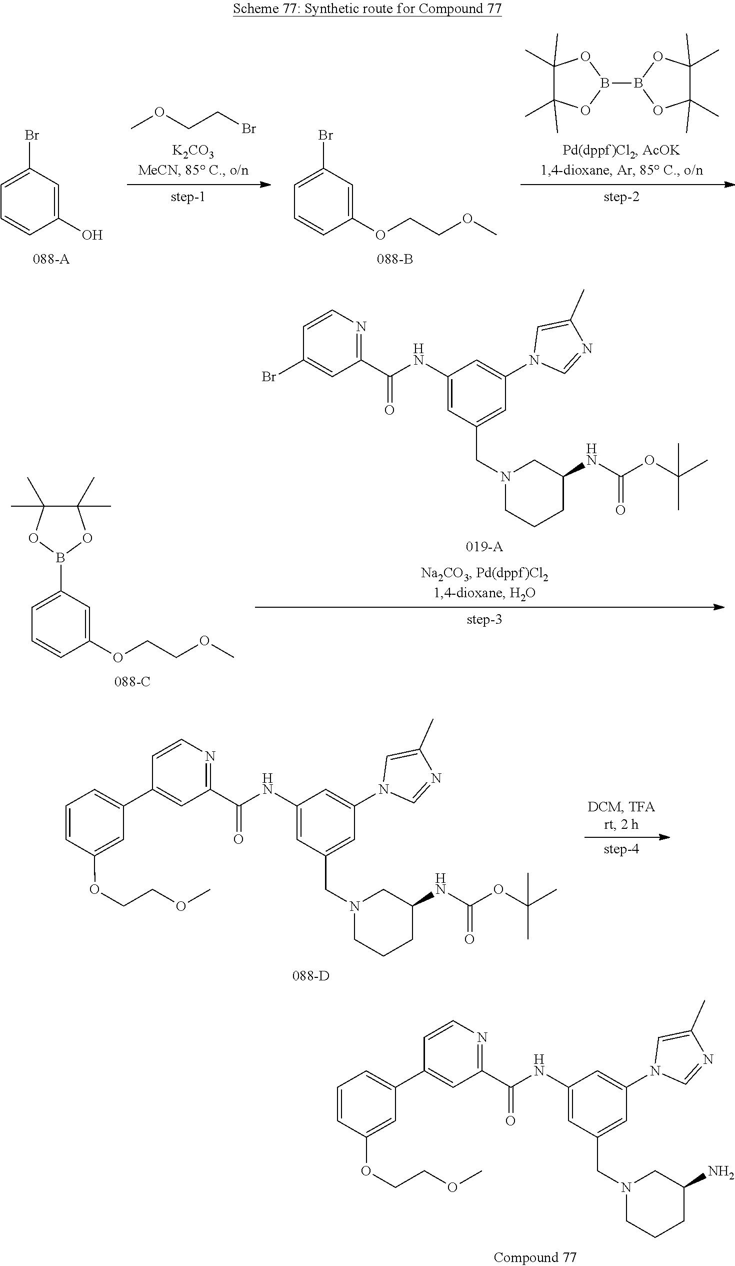

Step-1 The synthesis of 088-B: The title compound 088-B (1.7 g, 7.3 mmol, 74% yield) was prepared according to the General Method B2 with 1-bromo-2-methoxyethane (1.39 g, 10 mmol), 3-bromophenol (1.73 g, 10 mmol), K₂CO₃ (2.1 g, 15 mmol) in CH₃CN (100 mL) at 85° C.

Step-2 The synthesis of 088-C): The title compound 088-C (2.0 g, 7.2 mmol, 98% yield) was prepared according to the General Method I with 088-B (1.7 g, 7.3 mmol), bis(pinacolato)diborane (2.8 g, 11 mmol), KOAc (1.43 g, 14.6 mmol) and Pd(dppf)Cl₂ (0.54 g, 0.73 mmol) in 1,4-dioxane (40 mL) at 85° C.

Step-3 The synthesis of 088-D: The title compound 088-D (144 mg, 0.22 mmol, 64% yield) was prepared according to the General Method F with 019-A (200 mg, 0.35 mmol), 088-C (146 mg, 0.53 mmol), Pd(dppf)Cl₂ (25 mg, 0.035 mmol) and K₂CO₃ (145 mg, 1.05 mmol) in 1, 4-dioxane (20 mL) and H₂O (4 mL). LC/MS (ESI): m/z=641.3 [M+1]⁺. RT=2.27 min

Step-4 The synthesis of Compound 77: The title compound Compound 77 (48 mg, 40% yield) was prepared according to the General Method G with 088-D (144 mg, 0.23 mmol) and TFA (3 mL) in DCM (10 mL).

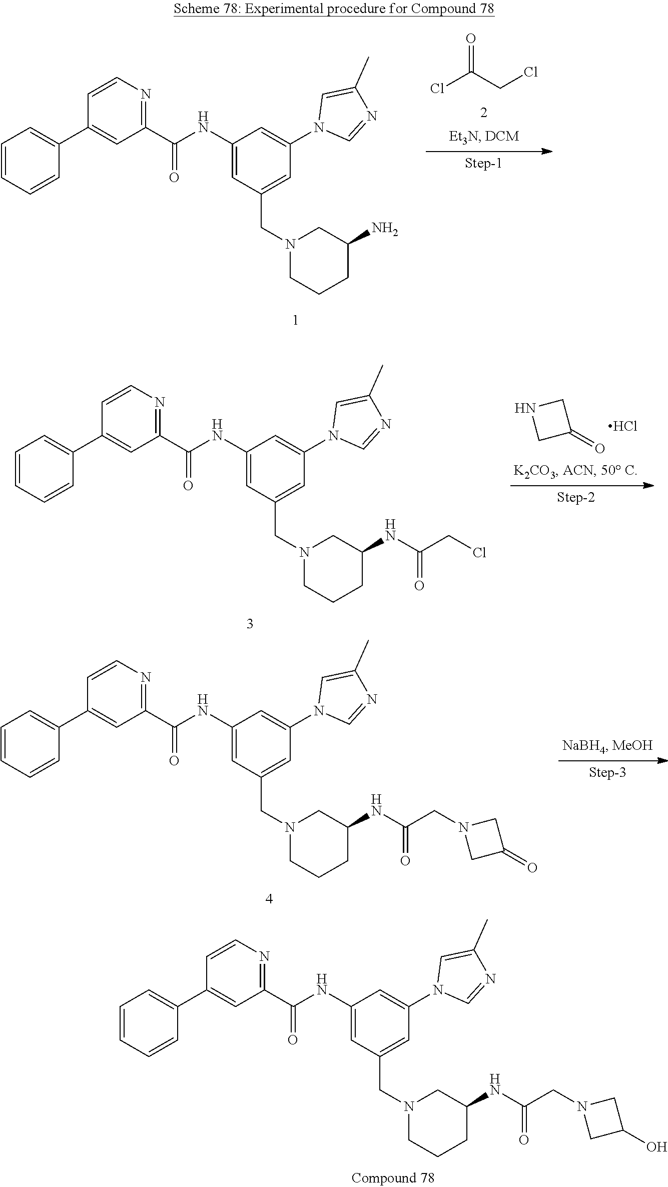

Step-1 Synthesis of N-(3-{[(3S)-3-(2-chloroacetamido)piperidin-1-yl]methyl}-5-(4-methyl-1H-imidazol-1-yl)phenyl)-4-phenylpyridine-2-carboxamide (3): To the stirred solution of 1 (300 mg, 0.596 mmol) in dichloromethane (20.0 mL), triethylamine (0.431 mL, 2.98 mmol) was added to it at 0° C. The reaction mass was stirred for 5 min at same temperature then 2-chloroacetyl chloride (2, 71.7 μL, 0.895 mmol) was added at 0° C. The resulting mixture was stirred at same temperature for 10 min. After consumption of starting material (monitored by TLC), the reaction mixture was diluted with water (10 mL) and aqueous layer was extracted with DCM (2×30 mL). The combined organic layer was dried over anhydrous sodium sulphate and concentrated under reduced pressure to give the title compound (3, 300 mg crude) as a brown gummy material which was forwarded to the next step without analysis.

Step-2 Synthesis of N-[3-(4-methyl-1H-imidazol-1-yl)-5-{[(3S)-3-[2-(3-oxoazetidin-1-yl)acetamido]piperidin-1-yl]methyl}phenyl]-4-phenylpyridine-2-carboxamide (4): To the stirred solution of 3 (300 mg, 0.552 mmol) and azetidin-3-one (238 mg, 2.21 mmol) in ACN (4.00 mL), K₂CO₃ (153 mg, 2 eq., 1.10 mmol) and potassium iodide (4.59 mg, 0.027 mmol) were added at room temperature and stirred the reaction mass at 80° C. for 6 h. After consumption of starting material (monitored by TLC), the reaction mixture was filtered through a plug of cotton and washed with EtOAc (2×20 mL). The combined organic layer was dried (anhydrous Na₂SO₄) and concentrated in vacuo to give the title compound (4, 150 mg crude) as a brown gum which was forwarded to the next step without analysis.

Step-3 Synthesis of (S)—N-(3-((3-(2-(3-hydroxyazetidin-1-yl)acetamido)piperidin-1-yl)methyl)-5-(4-methyl-1H-imidazol-1-yl)phenyl)-4-phenylpicolinamide (Compound 78): To the stirred solution of 4 (150 mg, 0.260 mmol) in EtOH (4.00 mL), sodium borohydride (37.2 mg, 1.04 mmol) was added at 0° C. and the reaction was stirred at room temperature for 2 h. After consumption of starting material (monitored by TLC), the reaction mixture was concentrated in vacuo and the residue was diluted with water (15 mL) and the aqueous layer was extracted with DCM (2×30 mL). The combined organic layer was dried (Na₂SO₄) and concentrated in vacuo. The residue was purified by T3 C18 (19×250) mm, 10 μm, gradient 50% to 50% ACN in water 0.1% TFA, RT: 12.30 min to give the title compound (Compound 78, 10.5 mg, 6.98%) as a white solid.

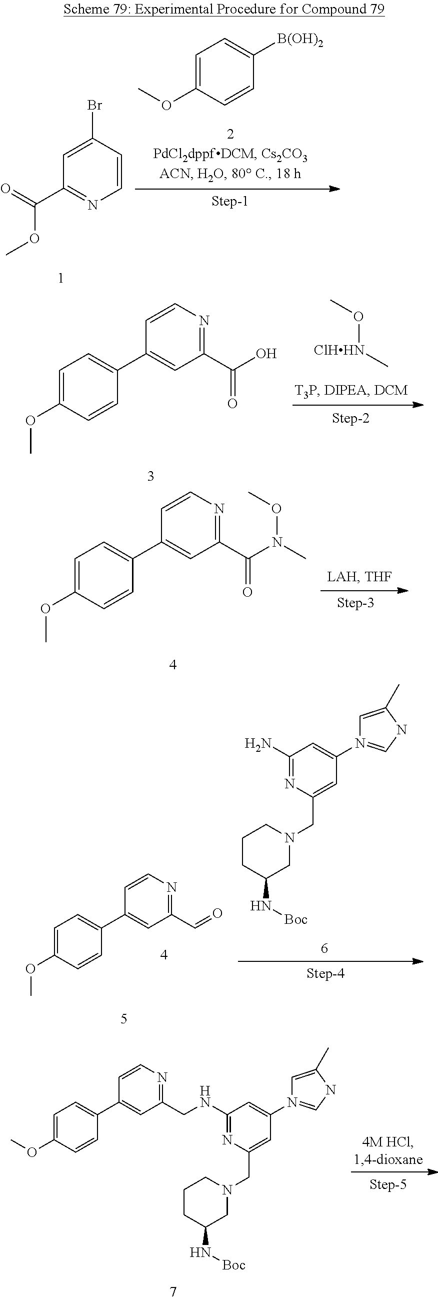

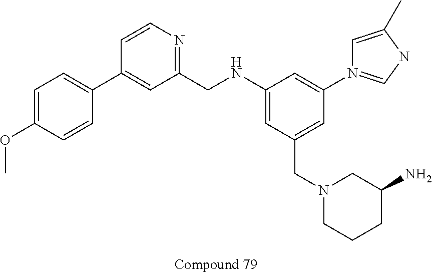

Step-1 Synthesis of 4-(4-methoxyphenyl)picolinic acid (3): To a stirred mixture of methyl 4-bromopyridine-2-carboxylate (1, 2.20 g, 10.2 mmol) and (4-methoxyphenyl)boronic acid (2, 1.39 g, 9.17 mmol) in ACN:H₂O (3:1, 4.0 mL), Cs₂CO₃ (6.60 g, 20.4 mmol) was added. The reaction mixture was degassed with Nitrogen for 10 minutes and PdCl₂dppf·DCM (0.73 g, 1.02 mmol) was added to it. The resulting reaction mixture was sealed with aluminium cap and stirred at 80° C. for 18 h. Progress of the reaction was monitored by TLC, which showed consumption of starting material. After completion of reaction, the reaction mixture was diluted with ethyl acetate and water (15 ml, 1:1) and filtered through a pad of celite. The organic layer was separated and the aqueous layer was neutralized with 1N HCl and light-brown precipitate was appeared which was filtered and dried in vacuo to give the title compound (3, 610, 26.1%) as a white solid. LCMS: m/z 230.1 (M+H)⁺ (ES+), 96%

Step-2 Synthesis of N-methoxy-4-(4-methoxyphenyl)-N-methylpicolinamide (4): To a solution of acid 3 (500 mg, 2.18 mmol) and methoxy(methyl)amine hydrochloride (319 mg, 3.27 mmol) in DCM (20 mL), DIPEA (846 mg, 6.54 mmol) was added. The reaction mixture was cooled to 0° C. and 1-propane phosphonic anhydride (50% solution in ethyl acetate, 2.04 mL and 3.27 mmol) was slowly added to the reaction mixture. The resulting mixture was stirred at room temperature for 2 h. After completion, the reaction mixture was quenched with aq NaHCO₃ (20 mL). After separating the organic layer, the aqueous layer was extracted with DCM (2×20 mL). The organic layers were combined, dried (Na₂SO₄) and concentrated in vacuo to give the title compound (4, 320 mg, 53%) as a white solid. LCMS: m/z 273.1 (M+H)⁺ (ES+), 99%

Step-3 Synthesis of 4-(4-methoxyphenyl)picolinaldehyde (5): To a stirred solution of 4 (0.5 g, 1.84 mmol) in dry THF (6.0 mL) and LAH (105 mg, 2.75 mmol) was added to it at 0° C. The reaction mixture was stirred at same temperature for 4 h. Progress of the reaction was monitored by TLC, which showed consumption of starting material. After completion of reaction, the reaction mixture was quenched with ammonium chloride solution (20 mL) and filter through a pad of celite and washed with EtOAc (2×30 mL). The organic layer was separated and aqueous layer was re-extracted with EtOAc (2×20 mL). The combined organic layer was dried (Na₂SO₄) and concentrated in vacuo to give the title compound (5, 410 mg crude) as a white solid. LCMS: m/z 214 (M+H)⁺ (ES+), 45%

Step-4 Synthesis of tert-butyl (S)-(1-((4-(4-methyl-1H-imidazol-1-yl)-6-(((4-phenylpyridin-2-yl)methyl)amino) pyridin-2-yl)methyl)piperidin-3-yl)carbamate (7): To a solution of 6 (633 mg, 1.64 mmol) and 4-(4-methoxyphenyl)picolinaldehyde (5, 350 mg, 1.64 mmol) in DCM (20 mL), Ti(OⁱPr)₄ (1.45 mL, 4.92 mmol) was added at 0° C. and the reaction mixture was stirred for 5 h. Na(OAc)₃BH (1.03 g, 4.92 mmol) was added portion wise to the reaction mixture at 0° C. and stirred at room temperature for 6 h. After completion, the reaction mixture was quenched with aqueous saturated NaHCO₃ and extracted with DCM (3×30 mL). The organic layers were combined and washed with brine, dried (Na₂SO₄) and concentrated in vacuo. The residue was purified by flash column chromatography [silica gel (100-200 mesh), gradient 1% to 2% MeOH in DCM] to enrich the purity up to 58% of the title compound 7 (220 mg, 55%) as a brown yellow solid. LCMS: m/z 583.61 (M+H)⁺ (ES+), 58%

Step-5 Synthesis of (S)-1-(3-(((4-(4-methoxyphenyl)pyridin-2-yl)methyl)amino)-5-(4-methyl-1H-imidazol-1-yl)benzyl)piperidin-3-amine (Compound 79): To a solution of 7 (220 mg, 0.378 mmol) in DCM (10 mL), 4 M HCl in dioxane (5 mL) was added. After completion (monitored by TLC), the reaction mixture was concentrated in vacuo. The crude material was purified by prep-HPLC (reverse phase, X Bridge shield C18 (19×250) mm, 10 μm, gradient 32% to 68% ACN in water containing 5 mM Ammonium acetate, RT: 10.95 min to give the title compound (Compound 79, 15 mg) as an off-white solid.

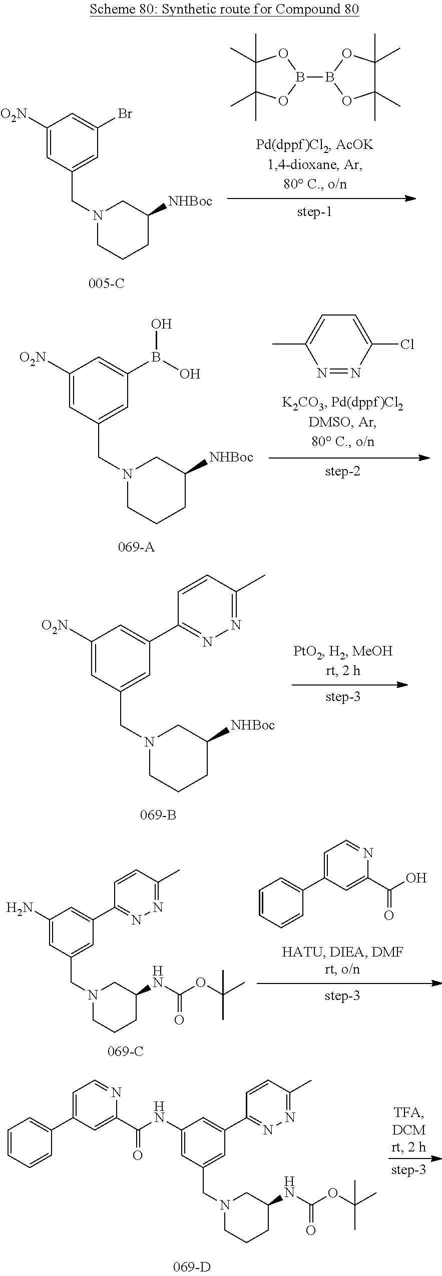

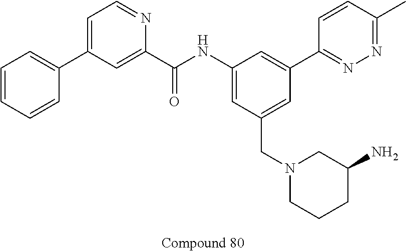 [SMILES: Cc1ccc(-c2cc(CN3CCC[C@H](N)C3)cc(NC(=O)c3cc(-c4ccccc4)ccn3)c2)nn1]

Step-1 The synthesis of 069-A: The title compound 069-A (345 mg, 0.91 mol, 75% yield) was prepared according to the General Method I with CP-005 (500 mg, 1.2 mmol), bis(pinacolato)diborane (457 mg, 1.8 mmol), KOAc (196 mg, 2.4 mmol) and Pd(dppf)Cl₂ (86 mg, 0.12 mmol) in 1,4-dioxane (25 mL) at 80° C. LC/MS (ESI): m/z=380.2 [M+1]⁺. RT=1.55 min

Step-2 The synthesis of 069-B: The title compound 069-B (145 mg, 0.34 mmol, 65% yield) was prepared according to the General Method F with 069-A (200 mg, 0.52 mmol), 3-chloro-6-methylpyridazine (66 mg, 0.52 mmol), Pd(dppf)Cl₂ (40 mg, 0.05 mmol) and K₂CO₃ (215 mg, 1.56 mmol) in DMSO (30 mL) at 80° C. LC/MS (ESI): m/z=428.0 [M+1]⁺. RT=1.91 min

Step-3 The synthesis of 069-C: The title compound 069-C (130 mg, 0.33 mmol, 96% yield) was prepared according to the General Method D1 with 069-B (145 mg, 0.34 mmol) and PtO₂ (40 mg) in MeOH (20 mL). LC/MS (ESI): m/z=398.4 [M+1]⁺. RT=1.75 min

Step-4 The synthesis of 069-D: The title compound 069-D (104 mg, 0.17 mmol, 55% yield) was prepared according to the General Method E with 069-C (130 mg, 0.33 mmol), 4-phenylpicolinic acid (66 mg, 0.33 mmol), HATU (188 mg, 0.5 mmol) and DIEA (129 mg, 1 mmol) in DMF (15 mL). LC/MS (ESI): m/z=579.0 [M+1]⁺. RT=2.11 min

Step-5 The synthesis of Compound 80: The title compound Compound 80 (20 mg, 25% yield) was prepared according to the General Method G with 069-D (104 mg, 0.18 mmol) and TFA (3 mL) in DCM (10 mL).

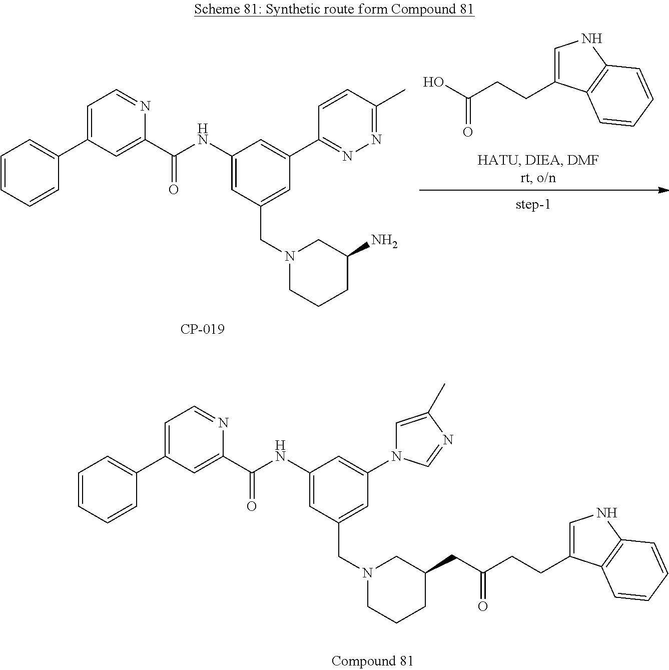

Step-1 The synthesis of Compound 81: The title compound Compound 81 (49 mg, 51% yield) was prepared according to the General Method E with CP-019 (70 mg, 0.15 mmol), 3-(1H-indol-3-yl)propanoic acid (87 mg, 0.23 mmol), HATU (60 mg, 0.23 mmol) and DIEA (78 mg, 0.60 mmol) in DMF (10 mL).

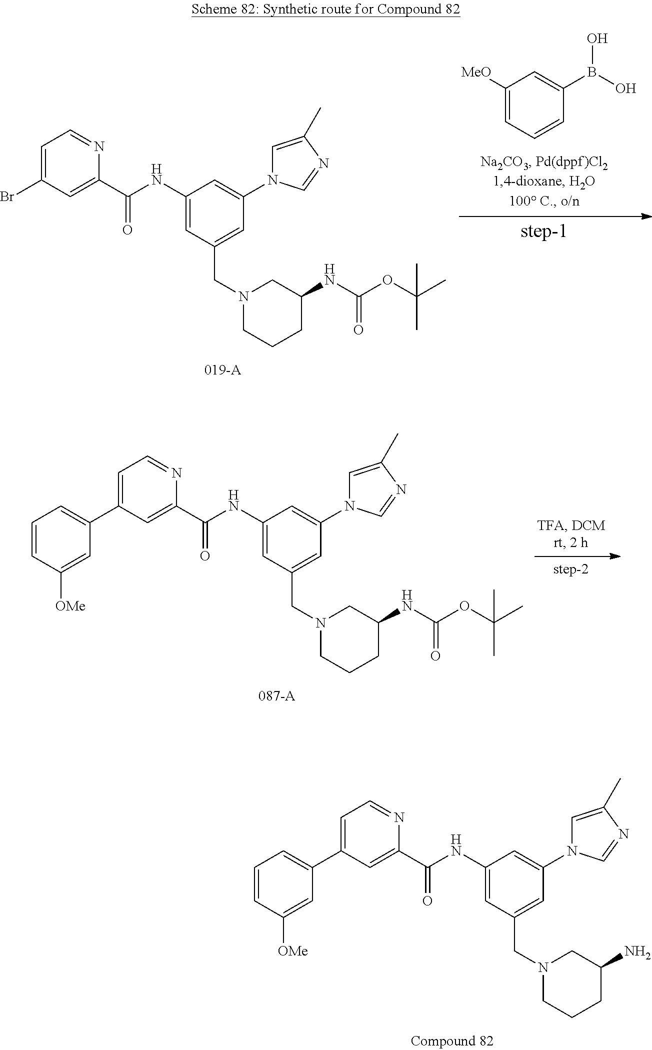 [SMILES: Cc1cn(-c2cc(CN3CCC[C@H](NC(=O)OC(C)(C)C)C3)cc(NC(=O)c3cc(Br)ccn3)c2)cn1 ; COc1cccc(B(O)O)c1 ; COc1cccc(-c2ccnc(C(=O)Nc3cc(CN4CCC[C@H](NC(=O)OC(C)(C)C)C4)cc(-n4cnc(C)c4)c3)c2)c1 ; COc1cccc(-c2ccnc(C(=O)Nc3cc(CN4CCC[C@H](N)C4)cc(-n4cnc(C)c4)c3)c2)c1]

Step-1 The synthesis of 087-A: The title compound 087-A (160 mg, 0.27 mmol, 76% yield) was prepared according to the General Method F with 019-A (200 mg, 0.35 mmol), (3-methoxyphenyl)boronic acid (80 mg, 0.53 mmol), Pd(dppf)Cl₂ (26 mg, 0.035 mmol) and Na₂CO₃ (112 mg, 1.05 mmol) in 1, 4-dioxane (15 mL) and H₂O (3 mL) at 100° C. LC/MS (ESI): m/z=597.0 [M+1]⁺. RT=2.11 min

Step-2 The synthesis of Compound 82: The title compound Compound 82 (65 mg, 49% yield) was prepared according to the General Method G with 087-A (160 mg, 0.27 mmol) and TFA (5 mL) in DCM (15 mL).

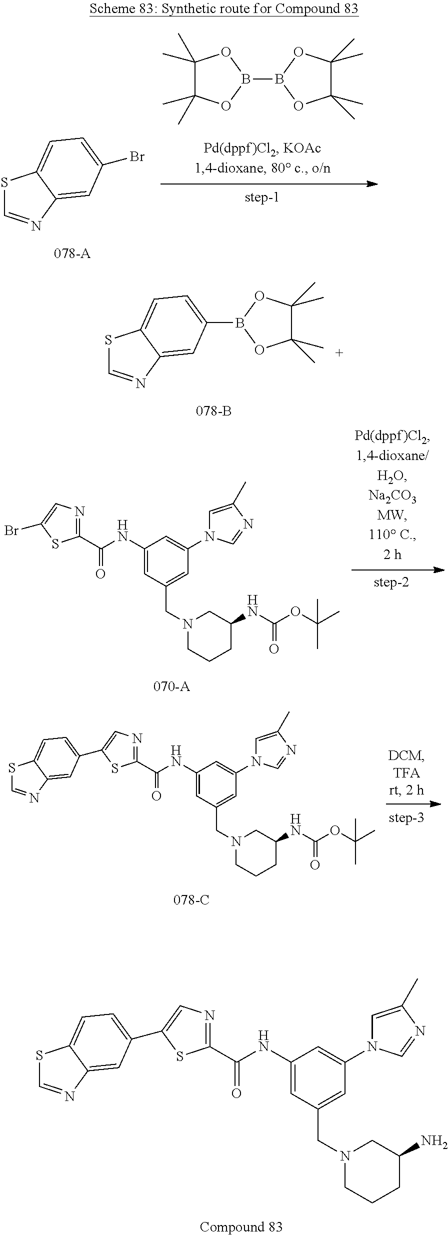

Step-1 The synthesis of 078-B: The title compound CP-078 (400 mg, 1.53 mmol, 65% yield) was prepared according to the General Method I with 078-A (500 mg, 2.35 mmol), bis(pinacolato)diborane (1.19 g, 4.70 mmol), Pd(dppf)Cl₂ (171 mg, 0.24 mmol) and KOAc (690 mg, 7.05 mmol) in 1, 4-dioxane (20 mL) at 80° C. LC/MS (ESI): m/z=262.0 [M+1]⁺. RT=1.74 min

Step-2 The synthesis of 078-C: General Method F1: To a mixture of 070-A (200 mg, 0.35 mmol), 078-B (136 mg, 0.53 mmol), and Na₂CO₃ (111 mg, 1.05 mmol) in 1,4-dioxane (2.5 mL) and H₂O (0.5 mL) was added Pd(dppf)Cl₂ (26 mg, 0.035 mmol), and the reaction mixture was purged with Ar, then stirred at 110° C. for 2 h under microwave irradiation. After cooling to room temperature, it was diluted by water (10 mL) and extracted with EtOAc (2×100 mL). The organic extract was washed by brine (20 mL), dried over anhydrous Na₂SO₄ and evaporated under reduced pressure to give a residue. The residue was purified by column chromatography on silica gel (eluting with 5% MeOH in DCM) to give 078-C (80 mg, 36% yield) as a yellow solid. LC/MS (ESI): m/z=629.8 [M+1]⁺. RT=2.01 min

Step-3 The synthesis of Compound 83: The title compound Compound 83 (14 mg, 21% yield) was prepared according to the General Method G with 078-C (80 mg, 0.13 mmol) and TFA (3 mL) in DCM (10 mL).

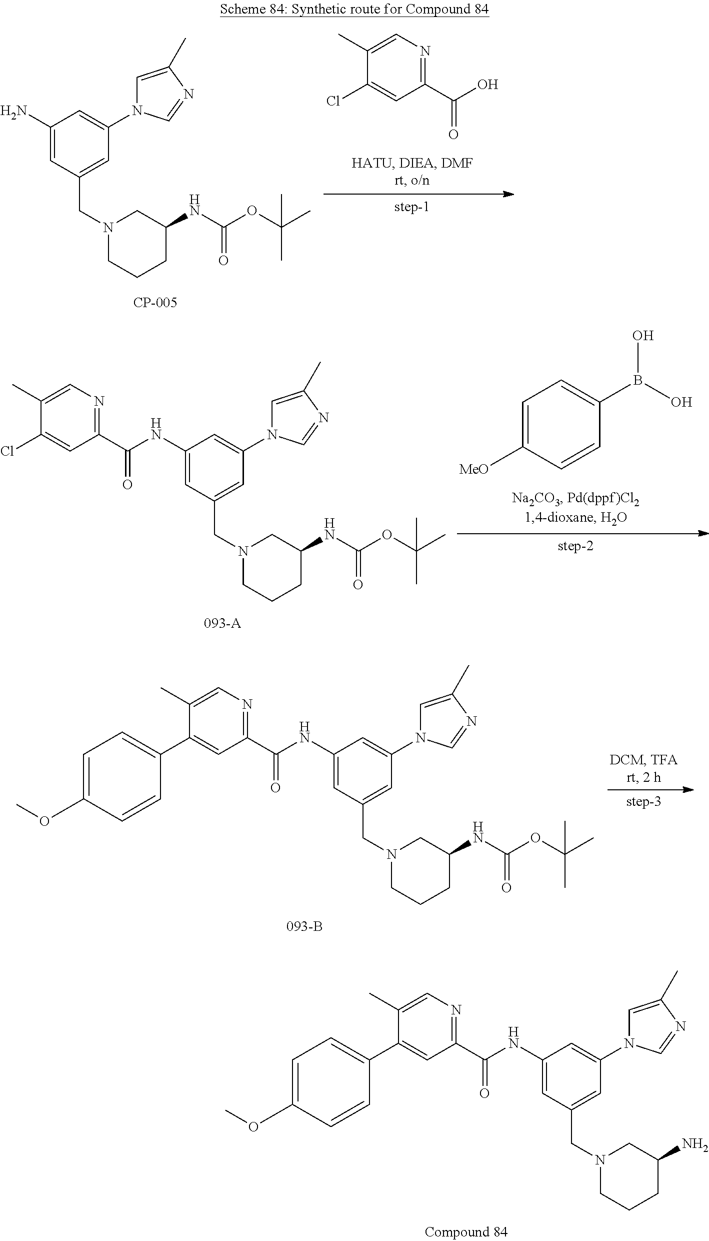

Step-1 The synthesis of 093-A: The title compound 093-A (140 mg, 0.26 mmol, 33% yield) was prepared according to the General Method E with CP-005 (300 mg, 0.78 mmol), 4-chloro-5-methylpicolinic acid (200 mg, 1.17 mmol), HATU (445 mg, 1.17 mmol) and DIEA (400 mg, 3.12 mmol) in DMF (15 mL). LC/MS (ESI): m/z=539.0 [M+1]⁺. RT=2.09 min

Step-2 The synthesis of 093-B: The title compound 093-B (90 mg, 0.15 mmol, 57% yield) was prepared according to the General Method F with 093-A (140 mg, 0.26 mmol), (4-methoxyphenyl)boronic acid (60 mg, 0.39 mmol), Pd(dppf)Cl₂ (19 mg, 0.026 mmol) and Na₂CO₃ (83 mg, 0.78 mmol) in 1, 4-dioxane (15 mL) and H₂O (3 mL) at reflux.

Step-3 The synthesis of Compound 84: The title compound Compound 84 (10 mg, 13% yield) was prepared according to the General Method G with 060-B (90 mg, 0.15 mmol) and TFA (5 mL) in DCM (15 mL).

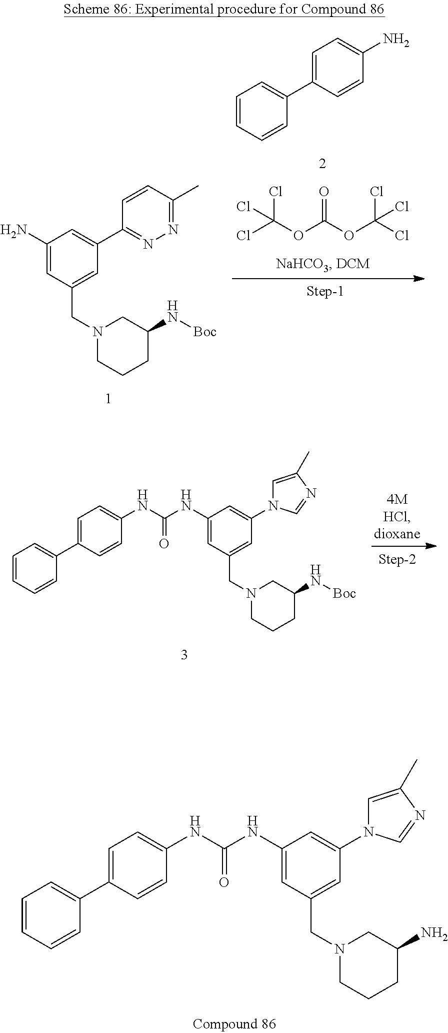 [SMILES: Nc1ccc(-c2ccccc2)cc1 ; Cc1ccc(-c2cc(N)cc(CN3CCC[C@H](NC(=O)OC(C)(C)C)C3)c2)nn1 ; O=C(OC(Cl)(Cl)Cl)OC(Cl)(Cl)Cl ; Cc1cn(-c2cc(CN3CCC[C@H](NC(=O)OC(C)(C)C)C3)cc(NC(=O)Nc3ccc(-c4ccccc4)cc3)c2)cn1 ; Cc1cn(-c2cc(CN3CCC[C@H](N)C3)cc(NC(=O)Nc3ccc(-c4ccccc4)cc3)c2)cn1]

Step-1 Synthesis of tert-butyl N-[(3S)-1-({3-[({[1,1′-biphenyl]-4-yl}carbamoyl)amino]-5-(4-methyl-1H-imidazol-1-yl)phenyl}methyl)piperidin-3-yl]carbamate (3): To a solution of amine 2 (100 mg, 591 μmol) in dichloromethane (6.00 mL), saturated aqueous NaHCO₃ solution (1.00 mL) was added. The reaction was stirred at 0° C. and triphosgene (49.3 mg, 0.295 mmol) was added dropwise. The reaction was allowed to stir for 1 h and solution of amine 1 (251 mg, 0.650 mmol) in DCM (4 mL) was added to the reaction mixture. The reaction was continued at room temperature for 16 h. After completion of reaction (monitored by TLC), water (30 mL) was added and the organic layer was extracted with DCM (3×50 mL). The organic layer was dried (Na₂SO₄) and concentrated in vacuo. The residue was purified by flash column chromatography [normal phase, silica gel (100-200 mesh), gradient 5% to 10% methanol in DCM] to give the title compound (3, 200 mg, 51%) as a yellow solid. LCMS: m/z 581.26 (M+H)⁺ (ES+), 89.68%

Step-2 Synthesis of 3-(3-{[(3S)-3-aminopiperidin-1-yl]methyl}-5-(4-methyl-1H-imidazol-1-yl)phenyl)-1-{[1,1′-biphenyl]-4-yl}urea (Compound 86): To a stirred solution of 3 in DCM (2.00 mL), 4M HCl in dioxane (5 mL) was added dropwise at 0° C. and the reaction mixture was stirred at room temperature for 1 h. After completion of reaction, the crude was concentrated in vacuo to give the residue. This residue was neutralized with NH₄OH solution (5 mL) and concentrated to dryness and purified by flash column chromatography [normal phase, silica gel (100-200 mesh), gradient 5% to 10% methanol in DCM] and the solid was further washed with pentane/diethyl ether to give the title compound (Compound 86, 44.0 mg, 48.33%) as a white solid.

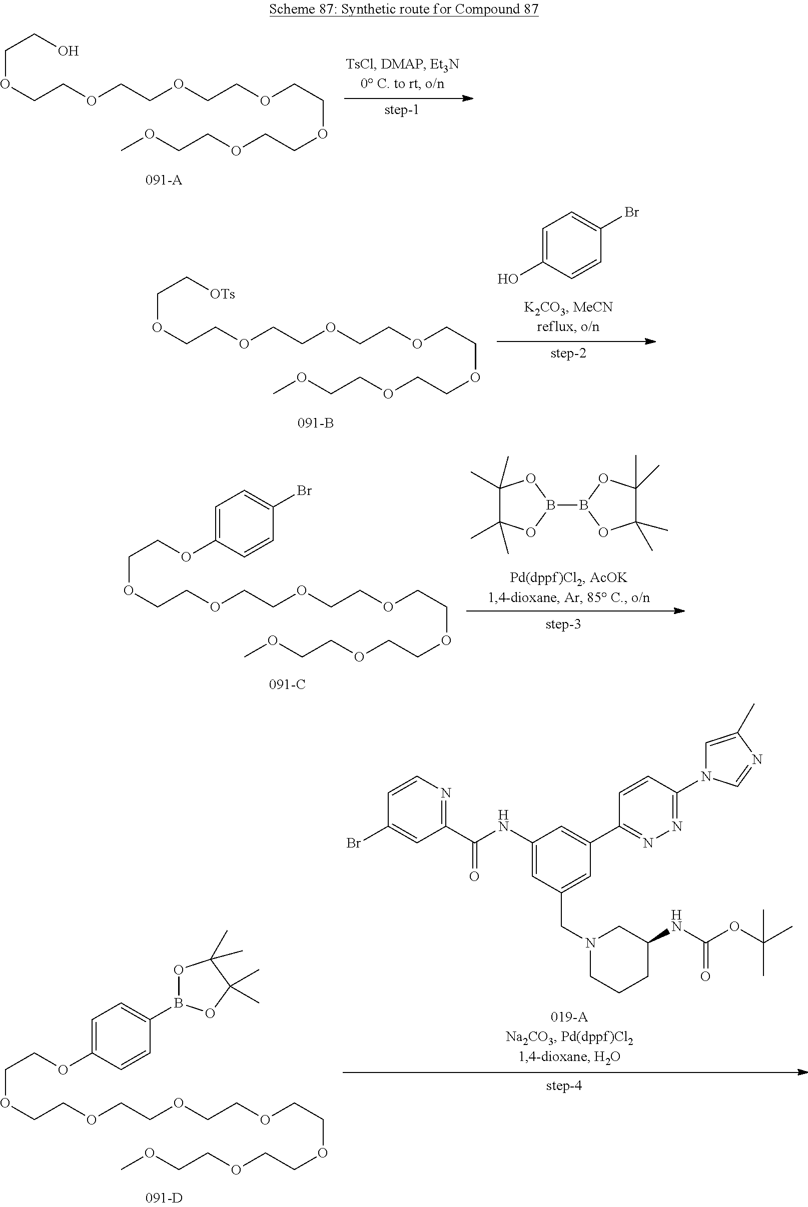

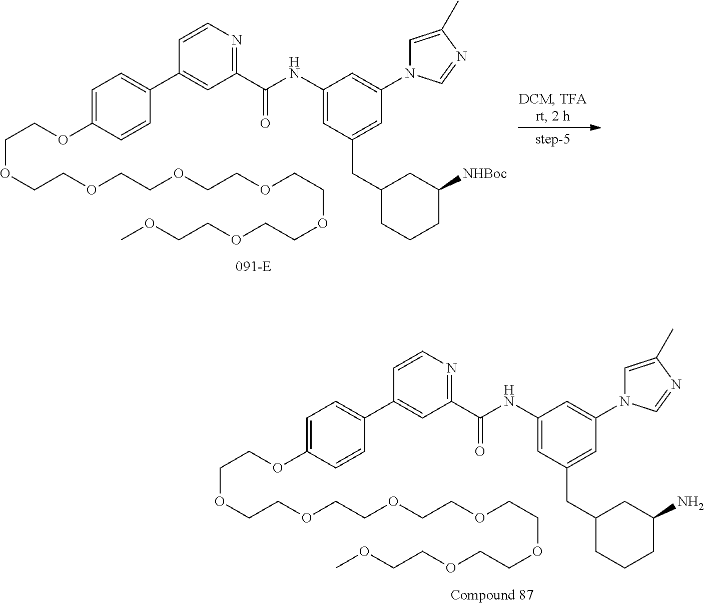 [SMILES: COCCOCCOCCOCCOCCOCCOCCOc1ccc(-c2ccnc(C(=O)Nc3cc(CC4CCC[C@H](NC(=O)OC(C)(C)C)C4)cc(-n4cnc(C)c4)c3)c2)cc1 ; COCCOCCOCCOCCOCCOCCOCCOc1ccc(-c2ccnc(C(=O)Nc3cc(CC4CCC[C@H](N)C4)cc(-n4cnc(C)c4)c3)c2)cc1]

Step-1 The synthesis of 091-B: The title compound 091-B (1.6 g, 3.24 mmol, 65% yield) was prepared according to the General Method K with 091-A (1.70 g, 5.0 mmol), TsCl (1.44 g, 6.0 mmol), Et₃N (1.4 mL, 10.0 mmol) and DMAP (61 mg, 0.5 mmol) in DCM (50 mL) at 0° C., and the reaction mixture was allowed to stir at room temperature overnight. LC/MS (ESI): m/z=512.0 [M+18]⁺. RT=1.71 min

Step-2 The synthesis of 091-C: The title compound 091-C (200 mg, 0.40 mmol, 80% yield) was prepared according to the General Method B2 with 091-B (280 mg, 0.55 mmol), 4-bromophenol (86.5 mg, 0.5 mmol) and K₂CO₃ (104 mg, 0.75 mmol) in CH₃CN (15 mL) at 85° C.

Step-3 The synthesis of 091-D: The title compound 091-D (410 mg, 0.76 mol, 53% yield) was prepared according to the General Method I with 091-C (620 mg, 1.25 mmol), bis(pinacolato)diborane (476 mg, 1.9 mmol), KOAc (245 mg, 2.5 mmol) and Pd(dppf)Cl₂ (91 mg, 0.125 mmol) in 1,4-dioxane (25 mL) and H₂O (5 mL). LC/MS (ESI): m/z=560.2 [M+1]⁺. RT=1.70 min

Step-4 The synthesis of 091-E: The title compound 091-E (110 mg, 0.12 mol, 35% yield) was prepared according to the General Method F with 019-A (200 mg, 0.35 mmol), 091-D (244 mg, 0.45 mmol), K₂CO₃ (97 mg, 0.7 mmol) and Pd(dppf)Cl₂ (30 mg, 0.035 mmol) in 1,4-dioxane (25 mL) and H₂O (5 mL). LC/MS (ESI): m/z=905.0 [M+1]⁺. RT=2.15 min

Step-5 The synthesis of Compound 87: The title compound Compound 87 (20 mg, 20% yield) was prepared according to the General Method G with 091-E (110 mg, 0.12 mmol) and TFA (3 mL) in DCM (10 mL).

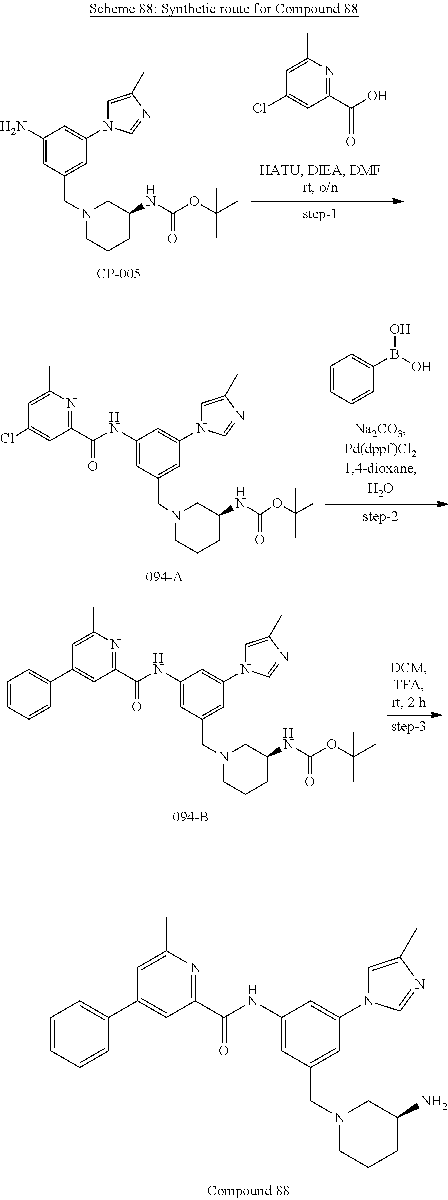 [SMILES: Cc1cn(-c2cc(N)cc(CN3CCC[C@H](NC(=O)OC(C)(C)C)C3)c2)cn1 ; Cc1cc(Cl)cc(C(=O)O)n1 ; Cc1cn(-c2cc(CN3CCC[C@H](NC(=O)OC(C)(C)C)C3)cc(NC(=O)c3cc(Cl)cc(C)n3)c2)cn1 ; OB(O)c1ccccc1 ; Cc1cn(-c2cc(CN3CCC[C@H](NC(=O)OC(C)(C)C)C3)cc(NC(=O)c3cc(-c4ccccc4)cc(C)n3)c2)cn1 ; Cc1cn(-c2cc(CN3CCC[C@H](N)C3)cc(NC(=O)c3cc(-c4ccccc4)cc(C)n3)c2)cn1]

Step-1 The synthesis of 094-A: The title compound 094-A (140 mg, 0.26 mmol, 30% yield) was prepared according to the General Method E with CP-005 (332 mg, 0.86 mmol), 4-chloro-6-methylpicolinic acid (147 mg, 0.86 mmol), HATU (4489 mg, 1.29 mmol) and DIEA (333 mg, 2.598 mmol) in DMF (5 mL). LC/MS (ESI): m/z=539.3 [M+1]⁺. RT=2.30 min

Step-2 The synthesis of 094-B: The title compound 094-B (60 mg, 0.10 mmol, 17% yield) was prepared according to the General Method F with 094-A (340 mg, 0.63 mmol), phenylboronic acid (128 mg, 1.89 mmol), Pd(dppf)Cl₂ (80 mg, 0.126 mmol) and Na₂CO₃ (164 mg, 2.5 mmol) in 1, 4-dioxane (25 mL) and H₂O (5 mL). LC/MS (ESI): m/z=581.3 [M+1]⁺. RT=2.42 min

Step-3 The synthesis of Compound 88: The title compound Compound 88 (17 mg, 14.4% yield) was prepared according to the General Method G with 094-B (140 mg, 0.24 mmol) and TFA (3 mL) in DCM (10 mL).

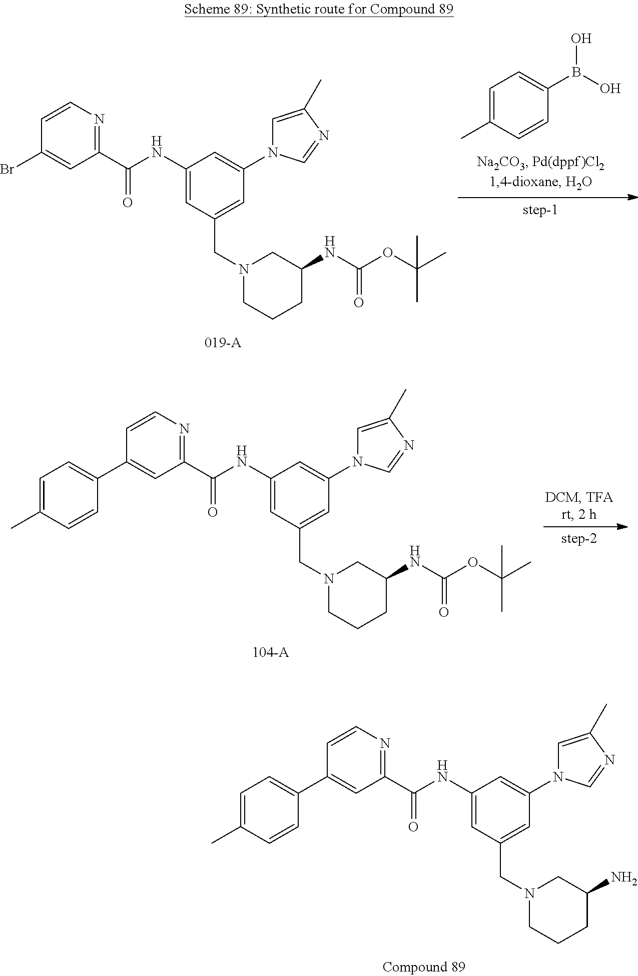

Step-1 The synthesis of 104-A: The title compound 104-A (135 mg, 0.23 mmol, 66% yield) was prepared according to the General Method F with 019-A (200 mg, 0.35 mmol), (4-methylphenyl)boronic acid (65 mg, 0.53 mmol), Pd(dppf)Cl₂ (26 mg, 0.036 mmol) and Na₂CO₃ (112 mg, 1.06 mmol) in 1, 4-dioxane (15 mL) and H₂O (3 mL). LC/MS (ESI): m/z=581.0 [M+1]⁺. RT=2.22 min

Step-2 The synthesis of Compound 89: The title compound Compound 89 (51 mg, 45% yield) was prepared according to the General Method G with 104-A (135 mg, 0.23 mmol) and TFA (5 mL) in DCM (15 mL).

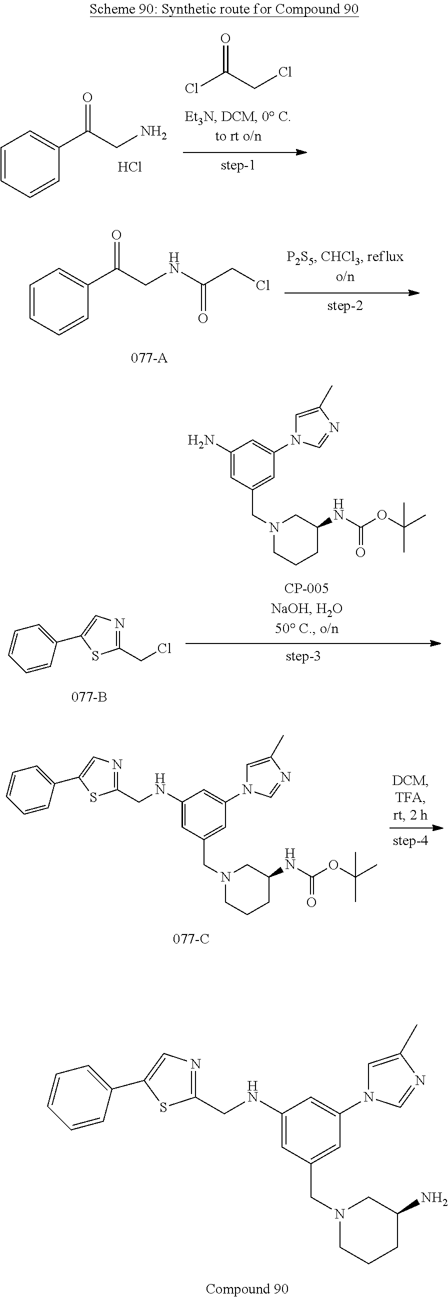

Step-1 The synthesis of 077-A: To a mixture of 2-amino-1-phenylethanone hydrochloride (1.72 g, 10.0 mmol) and 2-chloroacetyl chloride (0.95 mL, 12.0 mmol) in DCM (80 mL) was added Et₃N (4.2 mL, 30.0 mmol) slowly at 0° C., and the reaction mixture was allowed to stir at room temperature overnight. After consumption of the starting material, the reaction mixture was concentrated, then diluted by water (70 mL) and extracted with DCM (2×70 mL). The combined organic extract was washed by 2N HCl (100 mL) and brine (100 mL) successively, dried over anhydrous Na₂SO₄, filtered and evaporated under reduced pressure to give crude 077-A (1.66 g, 78% yield) as a yellow solid. LC/MS (ESI): m/z=212.2 [M+1]⁺. RT=1.51 min

Step-2 The synthesis of 077-B: To a solution of 077-A (846 mg, 4.0 mmol) in THF (30 mL) was added P₂S₅ (1.78 g, 8.0 mmol), and the reaction mixture was allowed to stir at reflux overnight. After consumption of the starting material, the reaction mixture was diluted by water (70 mL) and extracted with EtOAc (2×50 mL). The combined organic extract was washed by brine (50 mL), dried over anhydrous Na₂SO₄, filtered and evaporated under reduced pressure to give a residue. The residue was purified by column chromatography on silica gel (eluting with 4% EA in PE) to give 077-B (160 mg, 19% yield) as a yellow solid. LC/MS (ESI): m/z=210.2 [M+1]⁺. RT=0.75 min

Step-3 The synthesis of 077-C: To a mixture of CP-005 (300 mg, 0.78 mmol), 077-B (165 mg, 0.78 mmol) in H₂O (30 mL) was added NaOH (93 mg, 2.34 mmol), and the reaction mixture was stirred at 60° C. overnight. After consumption of the starting material, water (30 mL) was added and the mixture was extracted with EtOAc (2×50 mL). The combined organic layers were washed with brine (50 mL), dried over anhydrous Na₂SO₄ and concentrated under reduced pressure to give a residue. The residue was purified by Prep-TLC (eluting with 5% MeOH in DCM) and then reverse phase column chromatography to give 077-C (45 mg, 10.3% yield) as a yellow solid. LC/MS (ESI): m/z=559.0 [M+1]⁺. RT=2.01 min

Step-4 The synthesis of Compound 90: The title compound Compound 90 (20 mg, 54% yield) was prepared according to the General Method G with 077-C (45 mg, 0.080 mmol) and TFA (5 mL) in DCM (10 mL).

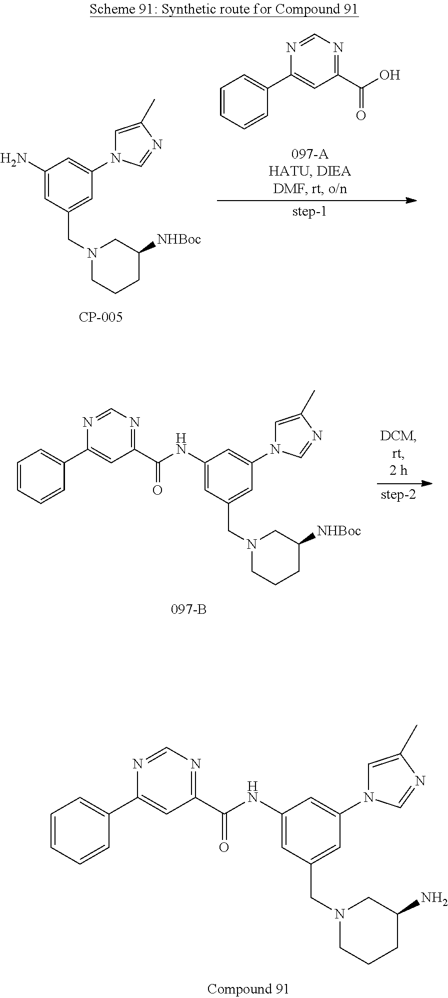 [SMILES: Cc1cn(-c2cc(N)cc(CN3CCC[C@H](NC(=O)OC(C)(C)C)C3)c2)cn1 ; O=C(O)c1cc(-c2ccccc2)ncn1 ; Cc1cn(-c2cc(CN3CCC[C@H](NC(=O)OC(C)(C)C)C3)cc(NC(=O)c3cc(-c4ccccc4)ncn3)c2)cn1 ; Cc1cn(-c2cc(CN3CCC[C@H](N)C3)cc(NC(=O)c3cc(-c4ccccc4)ncn3)c2)cn1]

Step-1 The synthesis of 097-B: The title compound 097-B (43 mg, 0.076 mmol, 9.7% yield) was prepared according to the General Method E with CP-005 (300 mg, 0.78 mmol), 6-phenylpyrimidine-4-carboxylic acid (155 mg, 0.78 mmol), HATU (385 mg, 1.01 mmol) and DIEA (300 mg, 2.34 mmol) in DMF (15 mL). LC/MS (ESI): m/z=568.3 [M+1]⁺. RT=2.28 min

Step-2 The synthesis of Compound 91: The title compound Compound 91 (18 mg, 50% yield) was prepared according to the General Method G with 097-B (43 mg, 0.76 mmol) and TFA (5 mL) in DCM (15 mL).

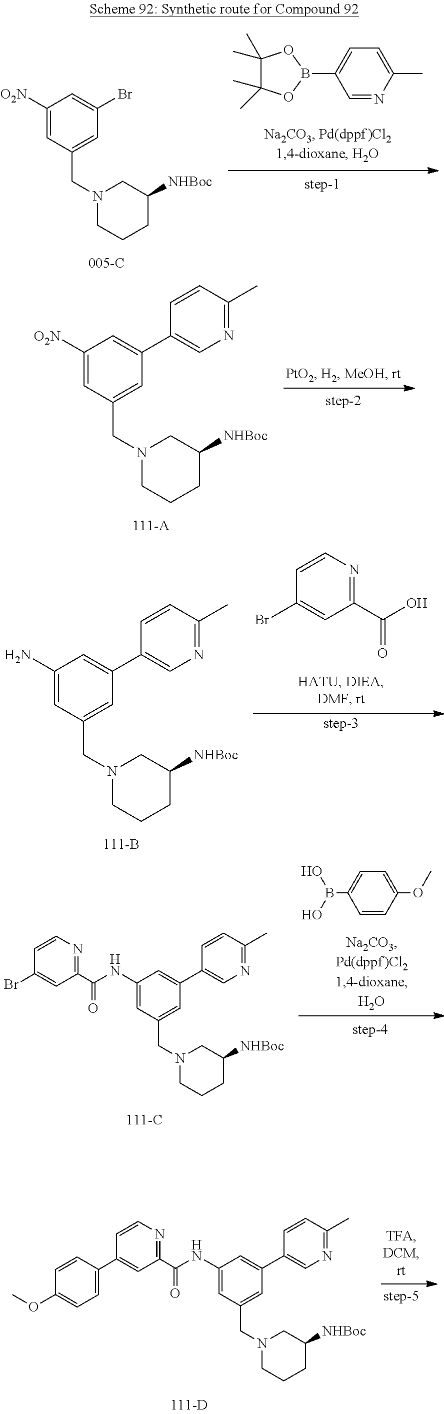

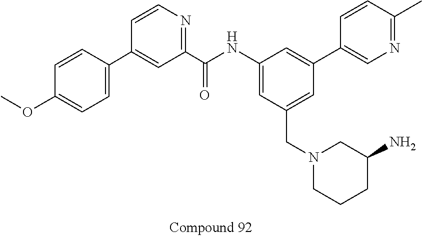

Step-1 The synthesis of 111-A: The title compound 111-A (930 mg, 2.08 mmol, 90% yield) was prepared according to the General Method F with 005-C (1 g, 2.42 mmol), 2-methyl-5-(4,4,5,5-tetramethyl-1,3,2-dioxaborolan-2-yl)pyridine (800 mg, 3.64 mmol), Pd(dppf)Cl₂ (177 mg, 0.24 mmol) and Na₂CO₃ (513 mg, 4.83 mmol) in 1, 4-dioxane (100 mL) and H₂O (20 mL). LC/MS (ESI): m/z=427.1 [M+1]⁺. RT=2.22 min

Step-2 The synthesis of 111-B: The title compound 111-B (510 mg, 1.29 mmol, 92.7% yield) was prepared according to the General Method D1 with 111-A (930 mg, 2.08 mmol), PtO₂ (93 mg) in MeOH (50 mL) under hydrogen atmosphere. LC/MS (ESI): m/z=397.1 [M+1]⁺. RT=1.91 min

Step-3 The synthesis of 111-C: The title compound 111-C (205 mg, 0.35 mmol, 70% yield) was prepared according to the General Method E with 111-B (200 mg, 0.51 mmol), 4-bromopicolinic acid (122 mg, 0.61 mmol), HATU (249.5 mg, 0.66 mmol) and DIEA (195.45 mg, 1.52 mmol) in DMF (20 mL). LC/MS (ESI): m/z=579.8 [M+1]⁺. RT=2.16 min

Step-4 The synthesis of 111-D: The title compound 111-D (185 mg, 0.30 mmol, 86% yield) was prepared according to the General Method F with 111-C (205 mg, 0.35 mmol), (4-methoxyphenyl)boronic acid (81 mg, 0.53 mmol), Pd(dppf)Cl₂ (26 mg, 0.036 mmol) and Na₂CO₃ (74.2 mg, 0.70 mmol) in 1,4-dioxane (20 mL) and H₂O (4 mL). LC/MS (ESI): m/z=608.0 [M+1]⁺. RT=2.23 min

Step-5 The synthesis of Compound 92: The title compound Compound 92 (81 mg, 53.6% yield) was prepared according to the General Method G with 111-D (185 mg, 0.30 mmol) and TFA (2 mL) in DCM (10 mL).

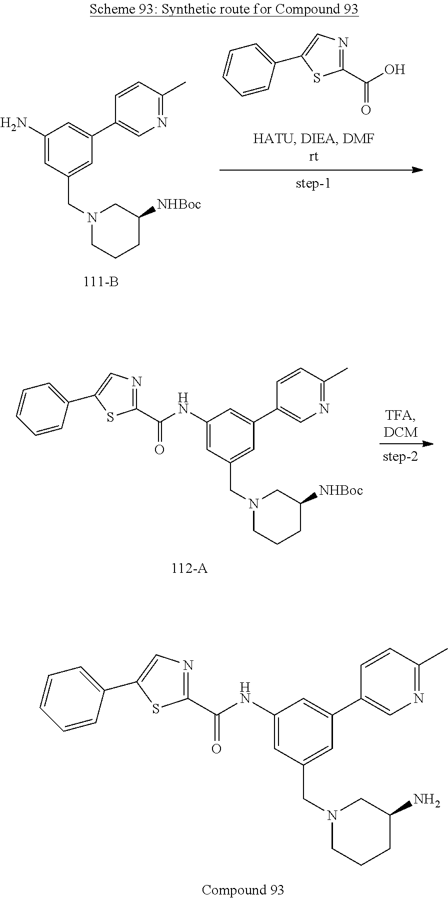

Step-1 The synthesis of 112-A: The title compound 112-A (138 mg, 0.24 mmol, 62.7% yield) was prepared according to the General Method E with 111-B (150 mg, 0.38 mmol), 5-phenylthiazole-2-carboxylic acid (94 mg, 0.46 mmol), HATU (187 mg, 0.49 mmol) and DIEA (147 mg, 1.14 mmol) in DMF (15 mL). LC/MS (ESI): m/z=584.0 [M+1]⁺. RT=2.02 min

Step-2 The synthesis of Compound 93: The title compound Compound 93 (54 mg, 47% yield) was prepared according to the General Method G with 112-A (138 mg, 0.24 mmol) and TFA (2 mL) in DCM (10 mL).

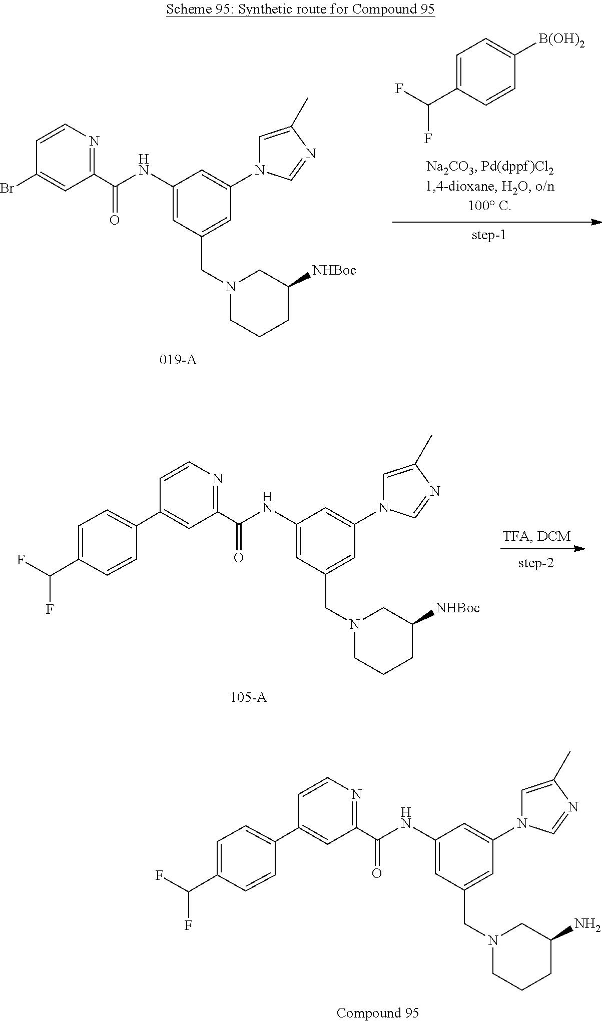 [SMILES: Cc1cn(-c2cc(CN3CCC[C@H](NC(=O)OC(C)(C)C)C3)cc(NC(=O)c3cc(Br)ccn3)c2)cn1 ; OB(O)c1ccc(C(F)F)cc1 ; Cc1cn(-c2cc(CN3CCC[C@H](NC(=O)OC(C)(C)C)C3)cc(NC(=O)c3cc(-c4ccc(C(F)F)cc4)ccn3)c2)cn1 ; Cc1cn(-c2cc(CN3CCC[C@H](N)C3)cc(NC(=O)c3cc(-c4ccc(C(F)F)cc4)ccn3)c2)cn1]

Step-1 The synthesis of 105-A: The title compound 105-A (211 mg, 0.37 mmol, 81% yield) was prepared according to the General Method F with 019-A (240 mg, 0.42 mmol), 4-(difluoromethyl)phenylboronic acid (110 mg, 0.64 mmol), Pd(dppf)Cl₂ (29 mg, 0.04 mmol) and Na₂CO₃ (134 mg, 1.26 mmol) in 1, 4-dioxane (20 mL) and H₂O (4 mL). LC/MS (ESI): m/z=617.0 [M+1]⁺. RT=1.91 min

Step-2 The synthesis of Compound 95: The title compound Compound 95 (39 mg, 20.6% yield) was prepared according to the General Method G with 105-A (211 mg, 0.37 mmol) and TFA (2 mL) in DCM (10 mL).

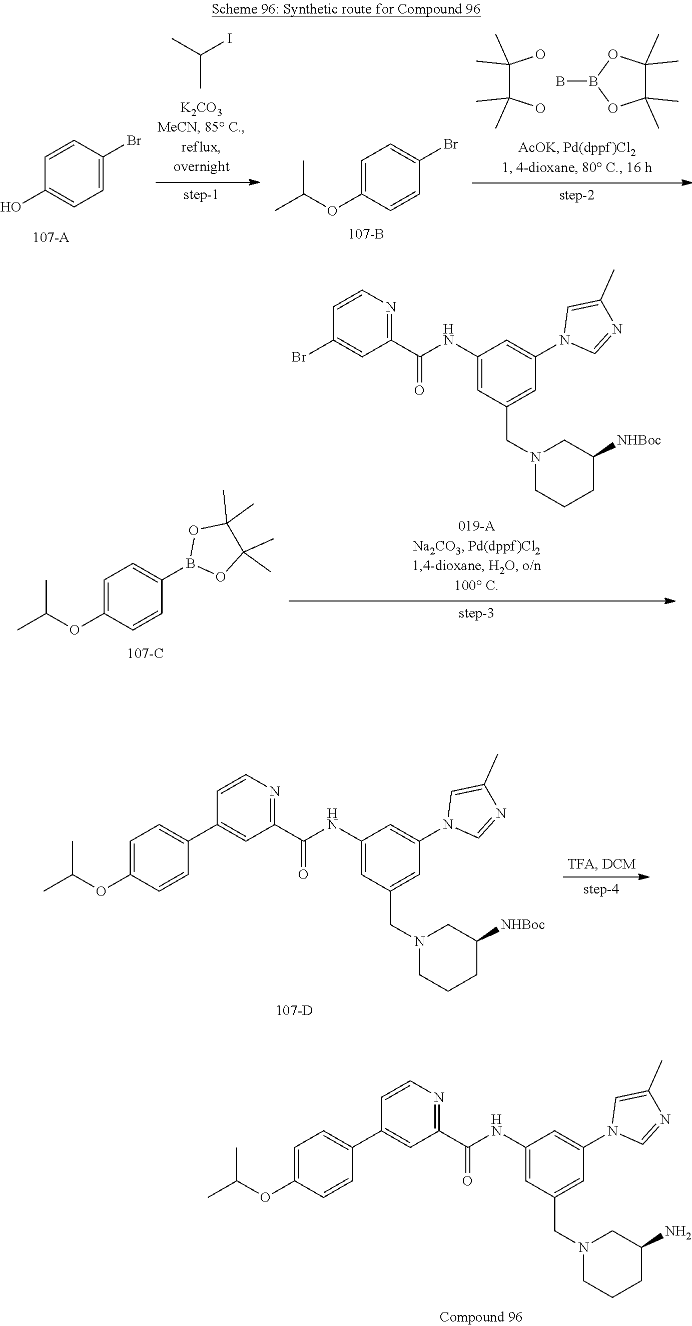

Step-1 The synthesis of 107-B: The title compound 107-B (1.2 g, 5.6 mmol, 99% yield) was prepared according to the General Method B2 with 4-bromophenol (1 g, 5.8 mmol), 2-iodopropane (2.96 g, 17.4 mmol), K₂CO₃ (1.2 g, 8.7 mmol) in CH₃CN (50 mL) at 85° C. overnight.

Step-2 The synthesis of 107-C: The title compound 107-C (675 mg, 2.58 mol, 50% yield) was prepared according to the General Method I with 107-B (1.1 g, 5.2 mmol), bis(pinacolato)diborane (1.97 mg, 7.8 mmol), KOAc (1.02 g, 10.4 mmol) and Pd(dppf)Cl₂ (0.38 g, 0.52 mmol) in 1,4-dioxane (30 mL) and H₂O (6 mL). LC/MS (ESI): m/z=263.0 [M+1]⁺. RT=1.98 min

Step-3 The synthesis of 107-D: The title compound 107-D (116 mg, 0.19 mmol, 53% yield) was prepared according to the General Method F with 019-A (200 mg, 0.35 mmol), 107-C (138 mg, 0.53 mmol), Pd(dppf)Cl₂ (25 mg, 0.035 mmol) and K₂CO₃ (145 mg, 1.05 mmol) in 1, 4-dioxane (25 mL) and H₂O (5 mL). LC/MS (ESI): m/z=624.8 [M+1]⁺. RT=2.03 min

Step-4 The synthesis of Compound 96: The title compound Compound 96 (18 mg, 19% yield) was prepared according to the General Method G with 107-D (116 mg, 0.19 mmol) and TFA (3 mL) in DCM (10 mL).

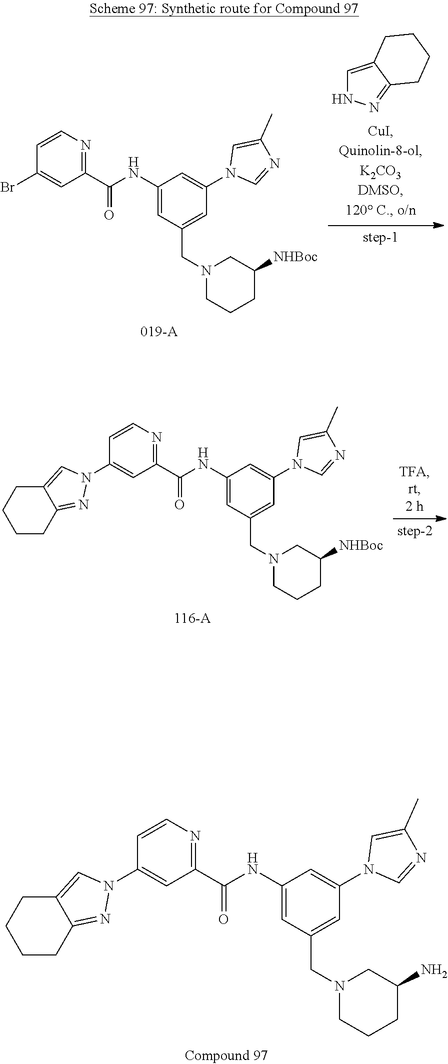 [SMILES: c1[nH]nc2c1CCCC2 ; Cc1cn(-c2cc(CN3CCC[C@H](NC(=O)OC(C)(C)C)C3)cc(NC(=O)c3cc(Br)ccn3)c2)cn1 ; Cc1cn(-c2cc(CN3CCC[C@H](NC(=O)OC(C)(C)C)C3)cc(NC(=O)c3cc(-n4cc5c(n4)CCCC5)ccn3)c2)cn1 ; Cc1cn(-c2cc(CN3CCC[C@H](N)C3)cc(NC(=O)c3cc(-n4cc5c(n4)CCCC5)ccn3)c2)cn1]

Step-1 The synthesis of 116-A: The title compound 116-A (200 mg, 0.33 mmol, 62% yield) was prepared according to the General Method C with 019-A (300 mg, 0.53 mmol), 4,5,6,7-tetrahydro-2H-indazole (180 mg, 1.5 mmol), K₂CO₃ (146 mg, 1.06 mmol), CuI (30.2 mg, 0.16 mmol) and quinolin-8-ol (16 mg, 0.11 mmol) in DMSO (10 mL). LC/MS (ESI): m/z=611.0 [M+1]⁺. RT=1.95 min

Step-2 The synthesis of Compound 97: The title compound Compound 97 (36 mg, 21% yield) was prepared according to the General Method G with 116-A (200 mg, 0.33 mmol) and TFA (3 mL) in DCM (10 mL).

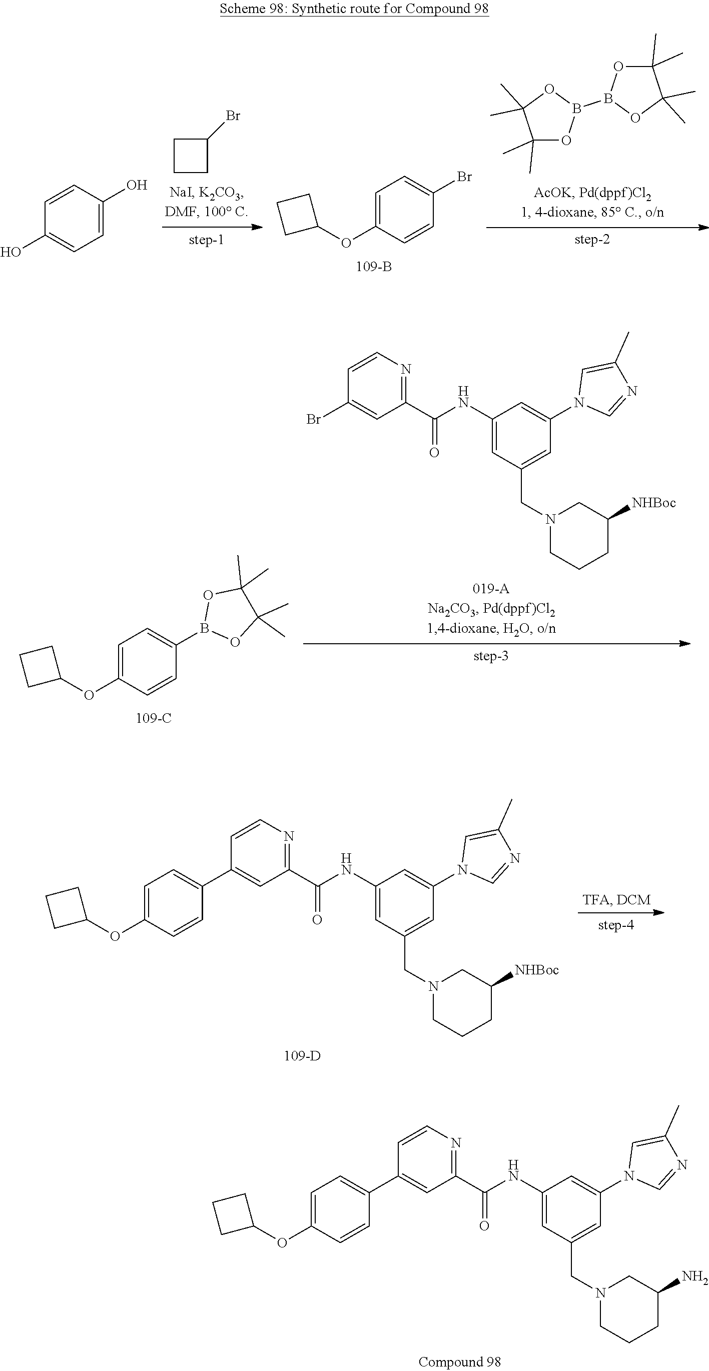

Step-1 The synthesis of 109-B: The title compound 109-B (337 mg, 1.49 mmol, 86% yield) was prepared according to the General Method B6 with 4-bromophenol (300 mg, 1.73 mmol), bromocyclobutane (468 mg, 3.49 mmol), NaI (26 mg, 0.17 mmol), K₂CO₃ (1.2 g, 8.69 mmol) in DMF (20 mL) at 100° C. overnight.

Step-2 The synthesis of 109-C: The title compound 109-C (377 mg, 1.38 mmol, 92% yield) was prepared according to the General Method I with 109-B (337 mg, 1.49 mmol), bis(pinacolato)diboron (758 mg, 2.98 mmol), Pd(dppf)Cl₂ (105 mg, 0.14 mmol) and KOAc (438 mg, 4.47 mmol) in 1, 4-dioxane (20 mL). LC/MS (ESI): m/z=275.0 [M+1]⁺. RT=2.05 min

Step-3 The synthesis of 109-D: The title compound 109-D (134 mg, 0.21 mmol, 50% yield) was prepared according to the General Method F with 109-C (140 mg, 0.51 mmol), 019-A (200 mg, 0.35 mmol), Pd(dppf)Cl₂ (52 mg, 0.071 mmol) and Na₂CO₃ (112 mg, 1.06 mmol) in 1, 4-dioxane (15 mL) and H₂O (3 mL). LC/MS (ESI): m/z=636.8 [M+1]⁺. RT=2.10 min

Step-4 The synthesis of Compound 109: The title compound Compound 109 (25 mg, 22% yield) was prepared according to the General Method G with 109-D (134 mg, 0.21 mmol) and TFA (2 mL) in DCM (10 mL).

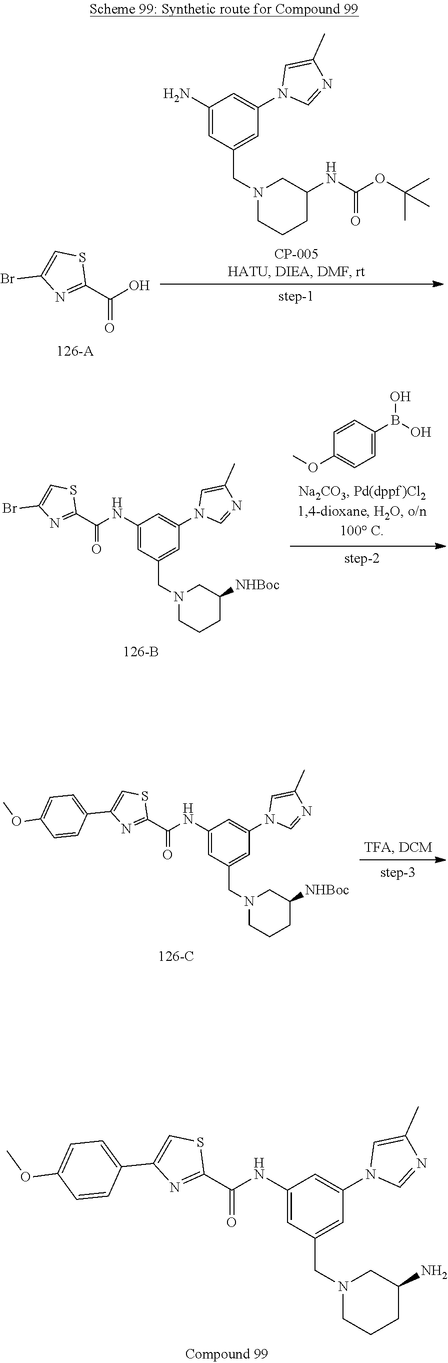 [SMILES: Cc1cn(-c2cc(N)cc(CN3CCCC(NC(=O)OC(C)(C)C)C3)c2)cn1 ; O=C(O)c1nc(Br)cs1 ; Cc1cn(-c2cc(CN3CCC[C@H](NC(=O)OC(C)(C)C)C3)cc(NC(=O)c3nc(Br)cs3)c2)cn1 ; COc1ccc(B(O)O)cc1 ; COc1ccc(-c2csc(C(=O)Nc3cc(CN4CCC[C@H](NC(=O)OC(C)(C)C)C4)cc(-n4cnc(C)c4)c3)n2)cc1 ; COc1ccc(-c2csc(C(=O)Nc3cc(CN4CCC[C@H](N)C4)cc(-n4cnc(C)c4)c3)n2)cc1]

Step-1 The synthesis of 126-B: The title compound 126-B (60 mg, 0.10 mmol, 20% yield) was prepared according to the General Method E with CP-005 (200 mg, 0.52 mmol), 4-bromothiazole-2-carboxylic acid (140 mg, 0.68 mmol), HATU (260 mg, 0.68 mmol) and DIEA (200 mg, 1.56 mmol) in DMF (15 mL). LC/MS (ESI): m/z=574.7 [M+1]⁺. RT=1.77 min

Step-2 The synthesis of 126-C: The title compound 126-C (60 mg, 0.10 mmol, 95% yield) was prepared according to the General Method F with 126-B (60 mg, 0.10 mmol), 4-methoxyphenylboronic acid (32 mg, 0.20 mmol), Pd(dppf)Cl₂ (7.6 mg, 0.01 mmol) and K₂CO₃ (43 mg, 0.30 mmol) in 1, 4-dioxane (16 mL) and H₂O (4 mL). LC/MS (ESI): m/z=602.8 [M+1]⁺. RT=1.88 min

Step-3 The synthesis of Compound 99: The title compound Compound 99 (25 mg, 50% yield) was prepared according to the General Method G with 126-C (60 mg, 0.1 mmol) and TFA (5 mL) in DCM (15 mL).

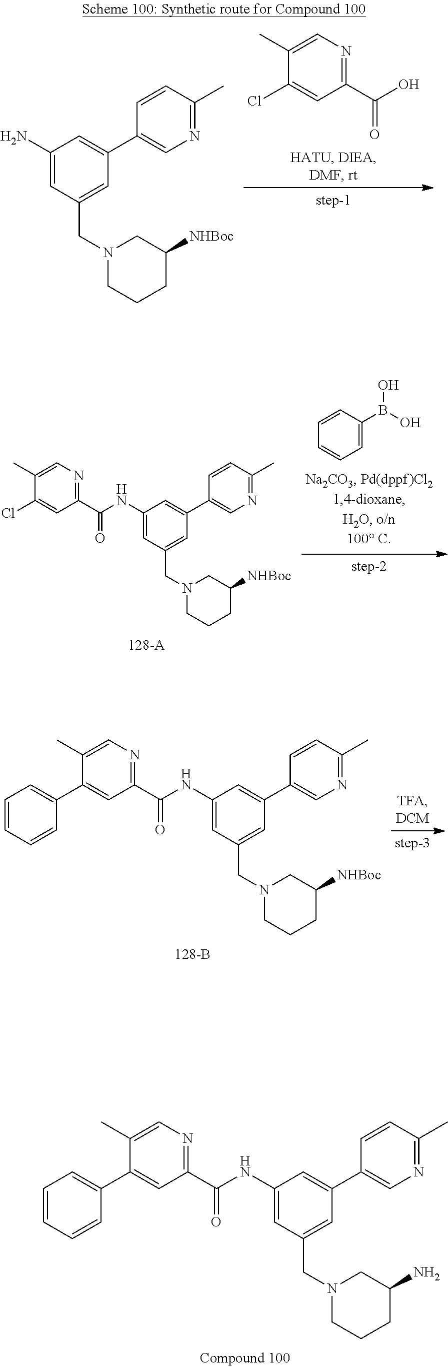

Step-1 The synthesis of 128-A: The title compound 128-A (290 mg, 0.53 mmol, 85% yield) was prepared according to the General Method E with (S)-tert-butyl 1-(3-amino-5-(6-methylpyridin-3-yl)benzyl)piperidin-3-ylcarbamate (245 mg, 0.62 mmol), 4-chloro-5-methylpicolinic acid (127 mg, 0.74 mmol), HATU (306 mg, 0.81 mmol) and DIEA (240 mg, 0.81 mmol) in DMF (20 mL). LC/MS (ESI): m/z=549.8 [M+1]⁺. RT=1.96 min

Step-2 The synthesis of 128-B: The title compound 128-B (102 mg, 0.17 mmol, 33% yield) was prepared according to the General Method F with 128-A (290 mg, 0.53 mmol), phenylboronic acid (65 mg, 0.53 mmol), Pd(dppf)Cl₂ (37 mg, 0.051 mmol) and Na₂CO₃ (169 mg, 1.59 mmol) in 1, 4-dioxane (15 mL) and H₂O (3 mL). LC/MS (ESI): m/z=591.8 [M+1]⁺. RT=2.03 min

Step-3 The synthesis of Compound 100: The title compound Compound 100 (49 mg, 59% yield) was prepared according to the General Method G with 128-B (102 mg, 0.17 mmol) and TFA (2 mL) in DCM (10 mL).

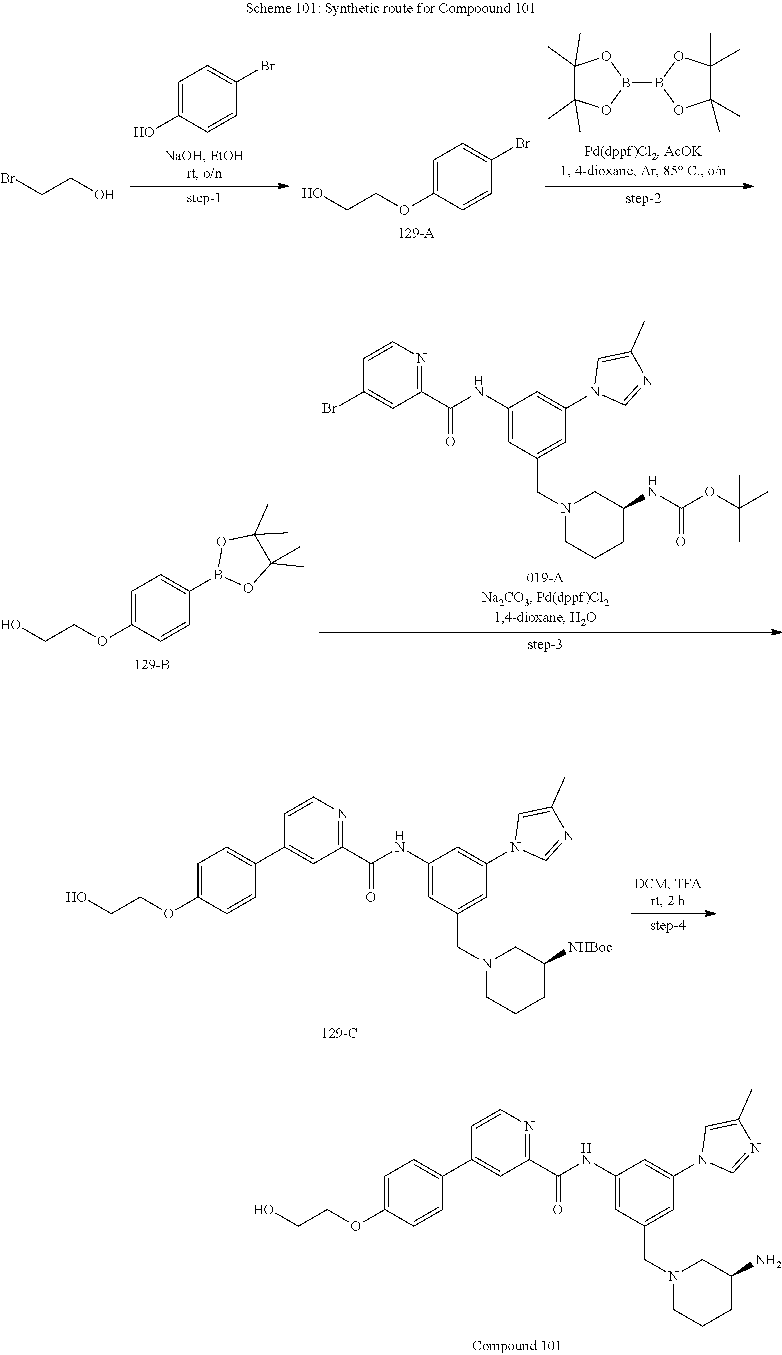

Step-1 The synthesis of 129-A: General Method B6: To a mixture of 4-bromophenol (1.73 g, 10.0 mmol), 2-bromoethanol (1.25 g, 10.0 mmol) in EtOH (30 mL) was added NaOH (440 mg, 11.0 mmol), and the reaction mixture was stirred at 60° C. overnight. After consumption of the starting material, water (50 mL) was added and the mixture was extracted with EtOAc (2×50 mL). The combined organic layers were washed with brine (50 mL), dried over anhydrous Na₂SO₄ and concentrated under reduced pressure to give the crude product 129-A (1.99 g, 91.7% yield) as a light-yellow oil, which was used directly to next step without further purification. LC/MS (ESI): m/z=217.0 [M+1]⁺. RT=0.59 min

Step-2 The synthesis of 129-B: The title compound 129-B (1.08 g, 4.09 mmol, 89% yield) was prepared according to the General Method I with 129-A (1.0 g, 4.61 mmol), bis(pinacolato)diborane (2.34 g, 9.22 mmol), KOAc (1.36 g, 13.83 mmol) and Pd(dppf)Cl₂ (340 mg, 0.46 mmol) in 1,4-dioxane (40 mL). LC/MS (ESI): m/z=265.2 [M+1]⁺. RT=0.66 min

Step-3 The synthesis of 129-C: The title compound 129-C (145 mg, 0.23 mmol, 46% yield) was prepared according to the General Method F with 019-A (285 mg, 0.50 mmol), 129-B (200 mg, 0.75 mmol), Pd(dppf)Cl₂ (36 mg, 0.05 mmol) and K₂CO₃ (207 mg, 1.50 mmol) in 1, 4-dioxane (20 mL) and H₂O (5 mL). LC/MS (ESI): m/z=626.8 [M+1]⁺. RT=1.69 min

Step-4 The synthesis of Compound 101: The title compound Compound 101 (45 mg, 37% yield) was prepared according to the General Method G with 129-C (145 mg, 0.23 mmol) and TFA (5 mL) in DCM (15 mL).

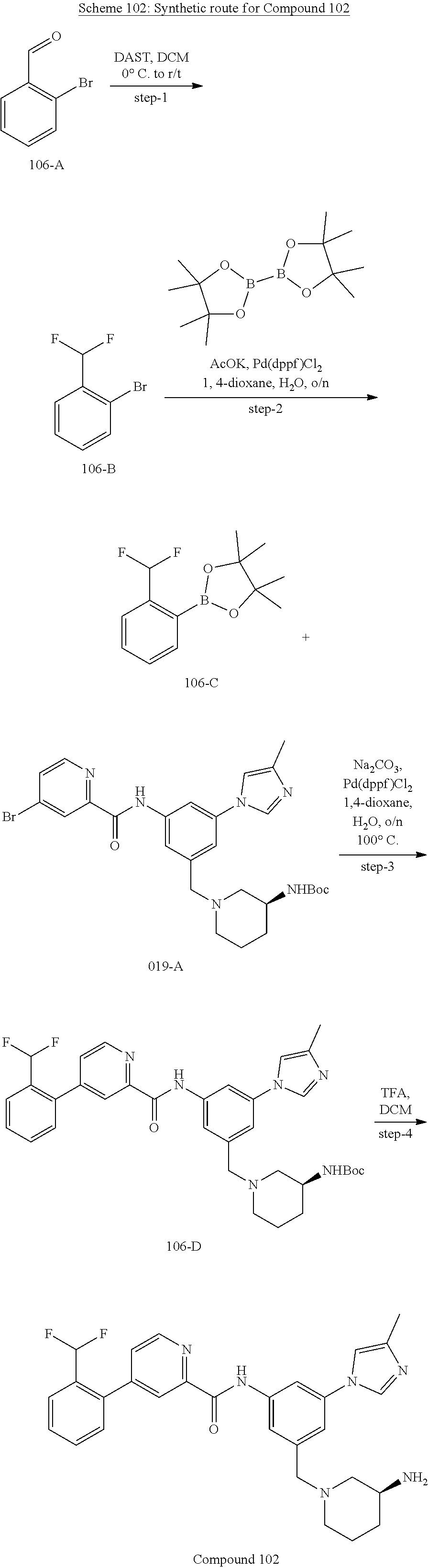

Step-1 The synthesis of 106-B: General Method O: To a solution of 2-bromobenzaldehyde (276 mg, 1.5 mmol) in DCM (15 mL) was added DAST (725 mg, 4.5 mmol) at 0° C., and the reaction mixture was allowed to warm to room temperature slowly and stir at room temperature overnight. After consumption of the starting material, the reaction mixture was concentrated, then diluted by water (50 mL) and extracted with DCM (2×40 mL). The organic extract was collected and washed by brine (50 mL), dried over anhydrous Na₂SO₄, filtered and evaporated under reduced pressure to give crude 106-B (240 mg, 77% yield) as a yellow gel.

Step-2 The synthesis of 106-C: The title compound 106-C (295 g, 1.16 mmol, 99% yield) was prepared according to the General Method I with 106-B (240 g, 1.16 mmol), bis(pinacolato)diborane (590 mg, 2.32 mmol), KOAc (342 mg, 3.48 mmol) and Pd(dppf)Cl₂ (85 mg, 0.12 mmol) in 1,4-dioxane (30 mL). LC/MS (ESI): m/z=255.3 [M+1]⁺. RT=0.77 min

Step-3 The synthesis of 106-D: The title compound 106-D (115 mg, 0.19 mmol, 35% yield) was prepared according to the General Method F with 019-A (300 mg, 0.53 mmol), 106-C (270 mg, 1.06 mmol), Pd(dppf)Cl₂ (77 mg, 0.10 mmol) and K₂CO₃ (218 mg, 1.58 mmol) in 1, 4-dioxane (20 mL) and H₂O (5 mL). LC/MS (ESI): m/z=616.9 [M+1]⁺. RT=2.32 min

Step-4 The synthesis of Compound 102: The title compound Compound 102 (56 mg, 58% yield) was prepared according to the General Method G with 106-D (115 mg, 0.19 mmol) and TFA (5 mL) in DCM (15 mL).

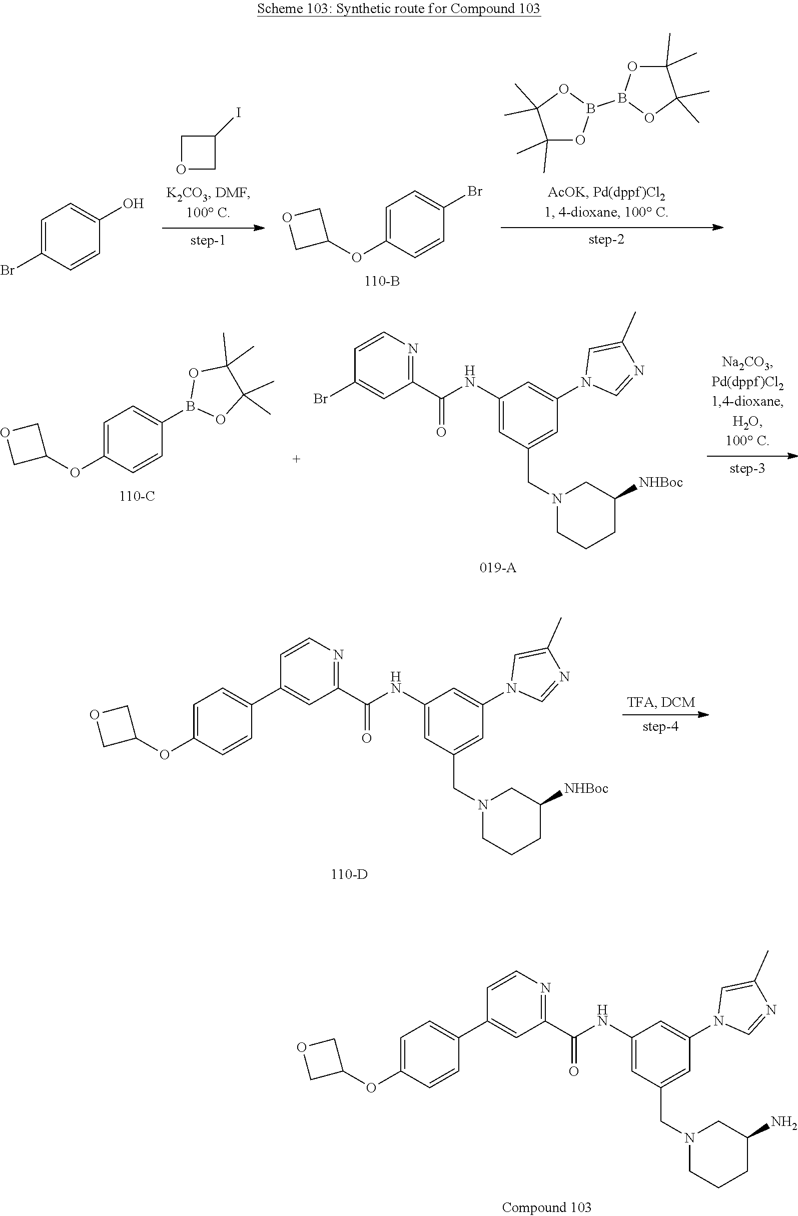

Step-1 The synthesis of 110-B: The title compound 110-B (409 mg, 1.79 mmol, 64% yield) was prepared according to the General Method B2 with 4-bromophenol (480 mg, 2.79 mmol), 3-bromooxetane (570 mg, 4.19 mmol), NaI (42 mg, 0.28 mmol), K₂CO₃ (1.15 g, 8.37 mmol) in DMF (30 mL) at 100° C. overnight.

Step-2 The synthesis of 110-C: The title compound 110-C (276 mg, 1.0 mmol, 56% yield) was prepared according to the General Method I with 110-B (409 mg, 1.79 mmol), bis(pinacolato)diboron (905 g, 3.56 mmol), Pd(dppf)Cl₂ (125 mg, 0.17 mmol) and KOAc (526 mg, 5.37 mmol) in 1, 4-dioxane (20 mL). LC/MS (ESI): m/z=277.0 [M+1]⁺. RT=1.92 min

Step-3 The synthesis of 110-D: The title compound 110-D (161 mg, 0.25 mmol, 48% yield) was prepared according to the General Method F with 110-C (276 mg, 1.0 mmol), 019-A (250 mg, 0.44 mmol), Pd(dppf)Cl₂ (65 mg, 0.089 mmol) and Na₂CO₃ (140 mg, 1.32 mmol) in 1, 4-dioxane (15 mL) and H₂O (3 mL). LC/MS (ESI): m/z=639.2 [M+1]⁺. RT=1.83 min

Step-4 The synthesis of Compound 103: The title compound Compound 103 (47 mg, 35% yield) was prepared according to the General Method G with 110-D (161 mg, 0.25 mmol) and TFA (2 mL) in DCM (10 mL).

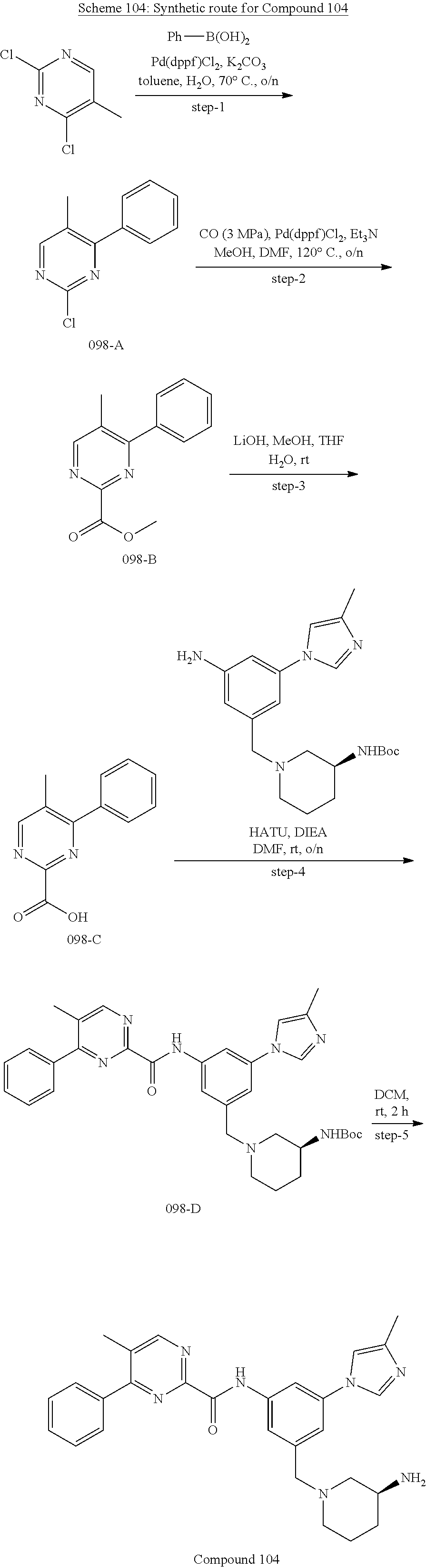

Step-1 The synthesis of 098-A: The title compound 098-A (823 mg, 4.03 mmol, 66% yield) was prepared according to the General Method F with 2,4-dichloro-5-methylpyrimidine (1.0 g, 6.13 mmol), phenylboronic acid (748 mg, 6.13 mmol), Pd(dppf)Cl₂ (446 mg, 0.61 mmol) and K₂CO₃ (1.69 g, 12.26 mmol) in toluene (40 mL) and H₂O (10 mL) at 70° C. LC/MS (ESI): m/z=205.2 [M+1]⁺. RT=1.61 min

Step-2 The synthesis of 098-B: General Method P: To a solution of 098-A (600 mg, 3.0 mmol) in MeOH (40 mL) and DMF (10 mL) was added Pd(dppf)Cl₂ (440 mg, 0.6 mmol) and Et₃N (2.5 mL), and the reaction mixture was purged with CO gas, heated to 120° C. and stirred overnight under CO atmosphere (3 MPa). After cooling to room temperature, it was diluted by water (100 mL) and extracted with EtOAc (2×100 mL). The organic extract was washed by water (4×100 mL) and brine (100 mL) successively, dried over anhydrous Na₂SO₄ and evaporated under reduced pressure to give a residue. The residue was purified by column chromatography on silica gel (eluting with 33% EA in PE) to give 098-B (85 mg, 12.5% yield) as light-red solid. LC/MS (ESI): m/z=229.3 [M+1]⁺. RT=1.60 min

Step-3 The synthesis of 098-C: The title compound 098-C (80 mg, 0.37 mmol, 99% yield) was prepared according to the General Method H with 098-B (85 mg, 0.37 mmol) and LiOH·H₂O (630 mg, 15.0 mmol) in THF (10 mL), MeOH (10 mL) and H₂O (10 mL). LC/MS (ESI): m/z=215.1 [M+1]⁺. RT=1.11 min

Step-4 The synthesis of 098-D: The title compound 098-D (80 mg, 0.14 mmol, 37% yield) was prepared according to the General Method E with CP-005 (215 mg, 0.56 mmol), 098-C (80 mg, 0.37 mmol), HATU (212 mg, 0.56 mmol) and DIEA (215 mg, 1.68 mmol) in DMF (10 mL). LC/MS (ESI): m/z=582.3 [M+1]⁺. RT=1.75 min

Step-5 The synthesis of Compound 104: The title compound Compound 104 (35 mg, 53% yield) was prepared according to the General Method G with 098-D (80 mg, 0.14 mmol) and TFA (5 mL) in DCM (15 mL).

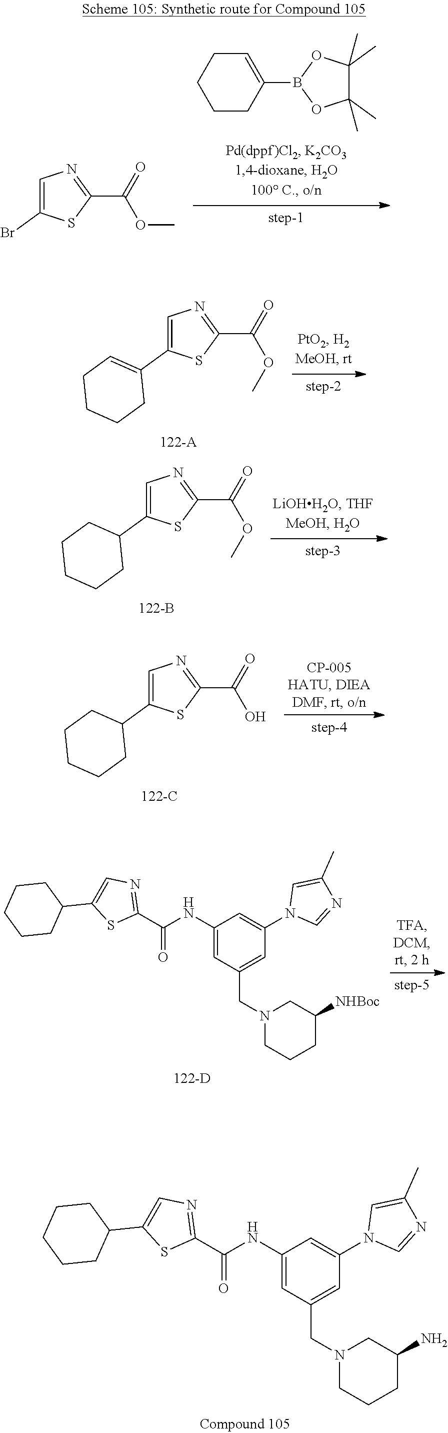

Step-1 The synthesis of 122-A: The title compound 122-A (283 mg, 1.27 mmol, 54% yield) was prepared according to the General Method F with methyl 5-bromothiazole-2-carboxylate (500 mg, 2.27 mmol), 2-cyclohexenyl-4,4,5,5-tetramethyl-1,3,2-dioxaborolane (708 mg, 3.41 mmol), Pd(dppf)Cl₂ (166 mg, 0.227 mmol) and K₂CO₃ (940 mg, 6.88 mmol) in 1, 4-dioxane (24 mL) and H₂O (6 mL). LC/MS (ESI): m/z=224.1 [M+1]⁺. RT=2.24 min

Step-2 The synthesis of 122-B: The title compound 122-B (260 mg, 1.16 mmol, 90% yield) was prepared according to the General Method D1 with 122-A (283 mg, 1.27 mmol) and PtO₂ (30 mg) in MeOH (20 mL). LC/MS (ESI): m/z=226.1 [M+1]⁺. RT=2.23 min

Step-3 The synthesis of 122-C: The title compound 122-C (160 mg, 0.20 mmol, 66% yield) was prepared according to the General Method H with 122-B (260 mg, 1.16 mmol), LiOH·H₂O (315 mg, 7.5 mmol) in THF (10 mL), MeOH (10 mL) and H₂O (10 mL). LC/MS (ESI): m/z=212.1 [M+1]⁺. RT=1.68 min

Step-4 The synthesis of 122-D: The title compound 122-D (65 mg, 0.11 mmol, 22% yield) was prepared according to the General Method E with 122-C (110 mg, 0.52 mmol), CP-005 (200 mg, 0.52 mmol), HATU (300 mg, 0.78 mmol) and DIEA (202 mg, 1.56 mmol) in DMF (25 mL). LC/MS (ESI): m/z=579.0 [M+1]⁺. RT=2.30 min

Step-5 The synthesis of Compound 105: The title compound Compound 105 (20 mg, 38% yield) was prepared according to the General Method G with 122-D (65 mg, 0.11 mmol) and TFA (3 mL) in DCM (15 mL).

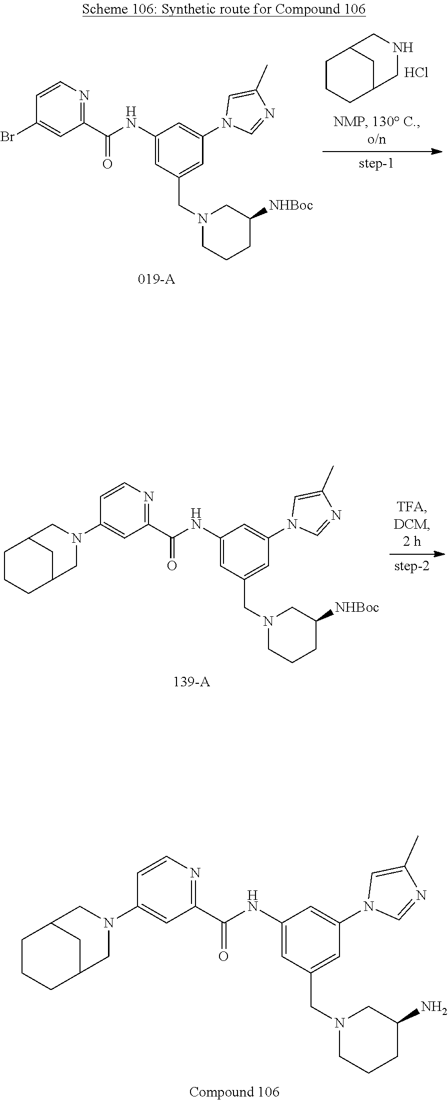 [SMILES: Cc1cn(-c2cc(CN3CCC[C@H](NC(=O)OC(C)(C)C)C3)cc(NC(=O)c3cc(Br)ccn3)c2)cn1 ; C1CC2CNCC(C1)C2 ; Cl ; Cc1cn(-c2cc(CN3CCC[C@H](NC(=O)OC(C)(C)C)C3)cc(NC(=O)c3cc(N4CC5CCCC(C5)C4)ccn3)c2)cn1 ; Cc1cn(-c2cc(CN3CCC[C@H](N)C3)cc(NC(=O)c3cc(N4CC5CCCC(C5)C4)ccn3)c2)cn1]

Step-1 The synthesis of 139-A: To a mixture of 019-A (285 mg, 0.50 mmol) and 3-azabicyclo[3.3.1]nonane hydrochloride (160 mg, 1.0 mmol) was added NMP (2 mL), and the reaction mixture was allowed to stir at 130° C. overnight. After consumption of the starting material, the reaction mixture was cooled to room temperature, diluted by water (20 mL) and extracted with EtOAc (2×20 mL). The organic extract was washed by water (4×50 mL) and brine (30 mL) successively, dried over anhydrous Na₂SO₄, filtered and evaporated under reduced pressure to give a residue. The residue was purified by Prep-TLC (eluting with DCM:MeOH=30:1) to give 139-A (200 mg, 65% yield) as a yellow solid. LC/MS (ESI): m/z=614.2 [M+1]⁺. RT=2.30 min

Step-2 The synthesis of Compound 106: To a solution of 139-A (200 mg, 0.33 mmol) in DCM (15 mL) was added TFA (5 mL), and the reaction mixture was stirred at room temperature for 2 h. After consumption of the starting material, the volatiles were evaporated under reduced pressure to give a residue. The resulted residue was dissolved in DCM (30 mL) and basified by aqueous K₂CO₃ (30 mL). The organic layer was separated, washed by water (30 mL) and brine (30 mL) successively, dried over anhydrous Na₂SO₄, filtered and evaporated under reduced pressure. The resulted residue was purified by Prep-HPLC to give Compound 106 (75 mg, 45% yield) as a white solid.

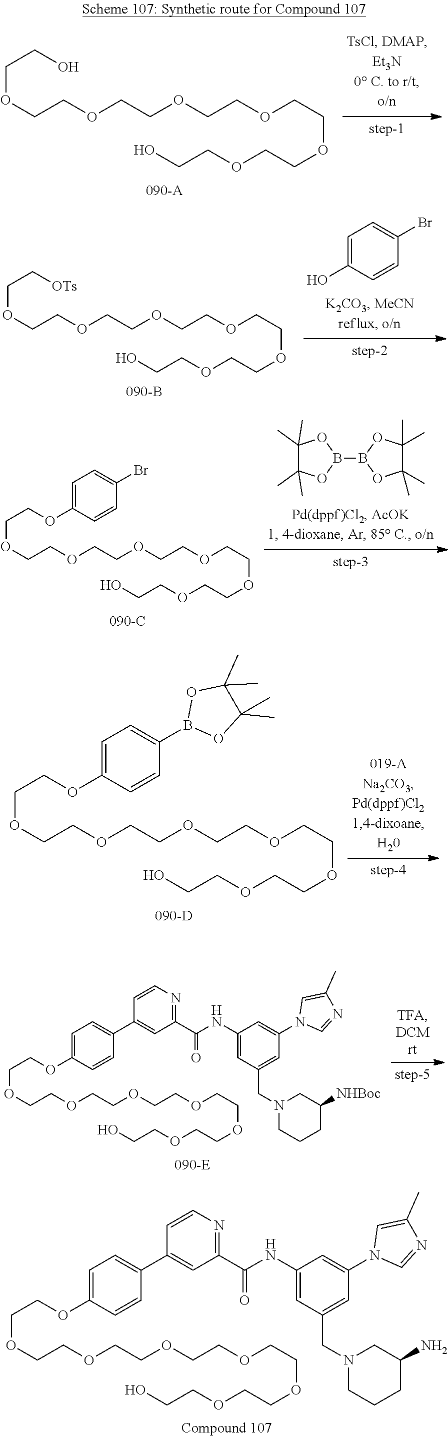

Step-1 The synthesis of 090-B: To a mixture of 090-A (3.55 g, 8.53 mmol), DMAP (112 mg, 0.921 mmol), and Et₃N (1.86 g, 18.42 mmol) in dry DCM (50 mL) was added TsCl (573 mg, 9.21 mmol) slowly at 0° C., and the reaction mixture was allowed to stir overnight under argon atmosphere at room temperature. After consumption of the starting materials, it was evaporated under reduced pressure to give a residue. The residue was dissolved in DCM (50 mL) and washed with brine (50 mL). The organic layer was dried over anhydrous Na₂SO₄ and evaporated under reduced pressure to give 090-B (880 mg, 1.83 mmol, 21.5% yield) as a colorless oil. LC/MS (ESI): m/z=498 [M+18]⁺. RT=1.376 min

Step-2 The synthesis of 090-C: The title compound 090-C (670 mg, 1.39 mmol, 76.1% yield) was prepared according to the General Method B2 with 090-B (880 mg, 1.83 mmol), 4-bromophenol (475 mg, 2.75 mmol) and K₂CO₃ (760 mg, 5.49 mmol) in CH₃CN (15 mL) at 85° C. LC/MS (ESI): m/z=498.2 [M+18]⁺. RT=1.465 min

Step-3 The synthesis of 090-D: The title compound 090-D (467 mg, 0.88 mol, 63.6% yield) was prepared according to the General Method I with 090-C (670 mg, 1.39 mmol), bis(pinacolato)diborane (686 mg, 2.7 mmol), AcOK (397 mg, 4.05 mmol) and Pd(dppf)Cl₂ (99 mg, 0.135 mmol) in 1,4-dioxane (25 mL) and H₂O (5 mL). LC/MS (ESI): m/z=546.3 [M+18]⁺. RT=1.576 min

Step-4 The synthesis of 090-E: The title compound 090-E (105 mg, 0.118 mol, 13.4% yield) was prepared according to the General Method F1 with 90-D (463 mg, 0.88 mol), 019-A (650 mg, 1.14 mmol), Na₂CO₃ (493 mg, 4.65 mmol) and Pd(dppf)Cl₂ (66 mg, 0.09 mmol) in 1,4-dioxane (60 mL) and H₂O (12 mL). LC/MS (ESI): m/z=454.8 [(M+18)/2+1]⁺. RT=1.70 min

Step-5 The synthesis of Compound 107: The title compound Compound 107 (5 mg, 5.3% yield) was prepared according to the General Method G with 090-E (105 mg, 0.12 mmol) and TFA (3 mL) in DCM (10 mL).

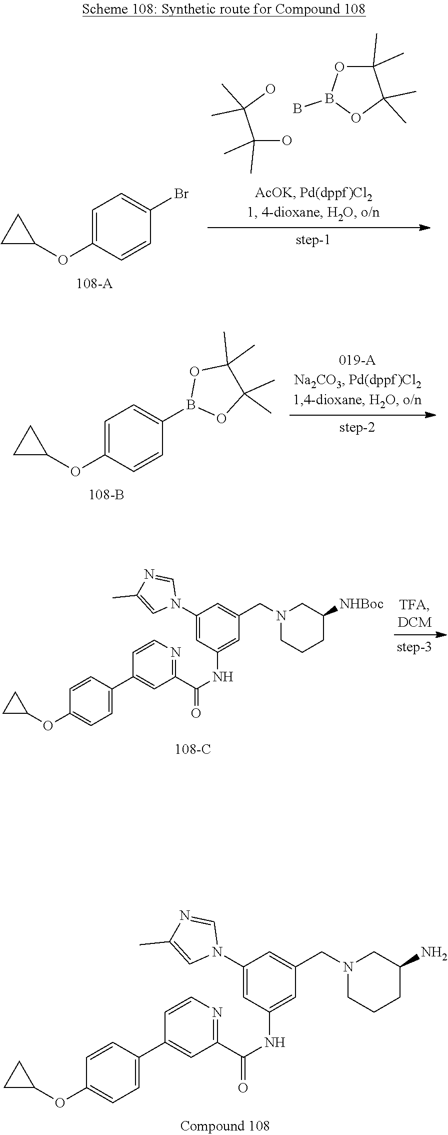

Step-1 The synthesis of 108-B: The title compound 108-B (700 mg, 2.69 mol, 57% yield) was prepared according to the General Method I with 108-A (1.0 g, 4.72 mmol), bis(pinacolato)diborane (3.57 g, 14 mmol), AcOK (920 mg, 9.39 mmol) and Pd(dppf)Cl₂ (691 mg, 0.94 mmol) in 1,4-dioxane (60 mL) and H₂O (12 mL). LC/MS (ESI): m/z=261.2 [M+1]⁺. RT=1.969 min

Step-2 The synthesis of 108-C: The title compound 108-C (267 mg, 0.43 mol, 15.99% yield) was prepared according to the General Method F2 with 108-B (700 mg, 2.69 mmol), 019-A (1.27 g, 2.24 mmol), Na₂CO₃ (712 mg, 6.72 mmol) and Pd(dppf)Cl₂ (328 mg, 0.45 mmol) in 1,4-dioxane (60 mL) and H₂O (12 mL). LC/MS (ESI): m/z=623 [M+1]⁺. RT=2.179 min

Step-3 The synthesis of Compound 108: The title compound Compound 108 (9 mg, 4% yield) was prepared according to the General Method G with 108-C (267 mg, 0.43 mmol) and TFA (3 mL) in DCM (10 mL).

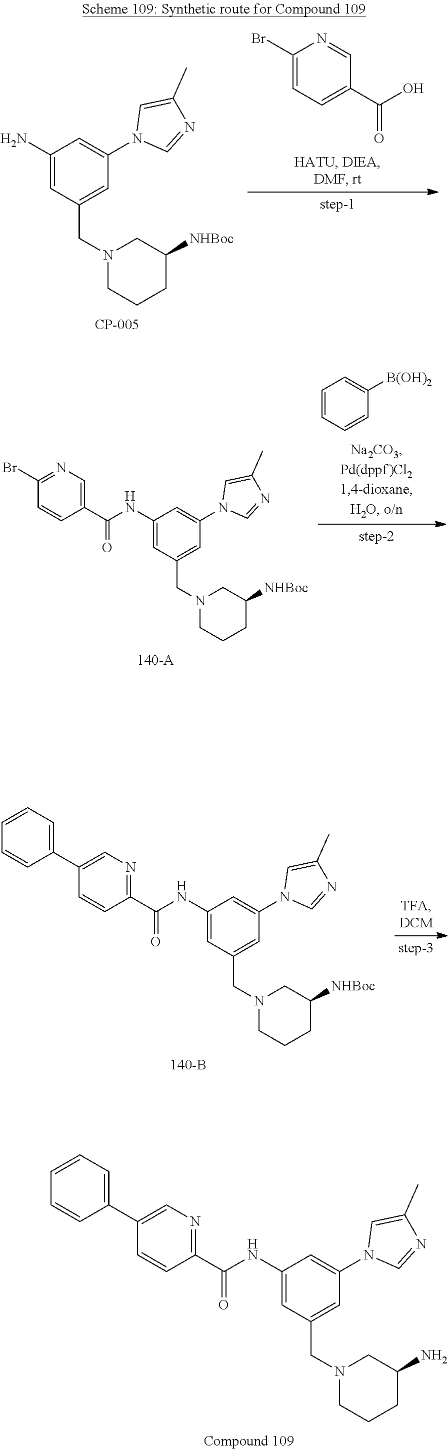 [SMILES: Cc1cn(-c2cc(N)cc(CN3CCC[C@H](NC(=O)OC(C)(C)C)C3)c2)cn1 ; O=C(O)c1ccc(Br)nc1 ; OB(O)c1ccccc1 ; Cc1cn(-c2cc(CN3CCC[C@H](NC(=O)OC(C)(C)C)C3)cc(NC(=O)c3ccc(Br)nc3)c2)cn1 ; Cc1cn(-c2cc(CN3CCC[C@H](NC(=O)OC(C)(C)C)C3)cc(NC(=O)c3ccc(-c4ccccc4)cn3)c2)cn1 ; Cc1cn(-c2cc(CN3CCC[C@H](N)C3)cc(NC(=O)c3ccc(-c4ccccc4)cn3)c2)cn1]

Step-1 The synthesis of 140-A: The title compound 140-A (190 mg, 0.33 mmol, 26% yield) was prepared according to the General Method E with CP-005 (500 mg, 1.30 mmol), 6-bromonicotinic acid (312 mg, 1.56 mmol), HATU (643 mg, 1.7 mmol) and DIEA (500 mg, 3.9 mmol) in DMF (15 mL). LC/MS (ESI): m/z=569.2 [M+1]⁺. RT=1.818 min

Step-2 The synthesis of 140-B: The title compound 140-B (112 mg, 0.198 mol, 60% yield) was prepared according to the General Method F1 with 140-A (190 mg, 0.33 mmol), phenylboronic acid (48 mg, 0.40 mmol), Na₂CO₃ (105 mg, 0.99 mmol) and Pd(dppf)Cl₂ (24 mg, 0.033 mmol) in 1,4-dioxane (60 mL) and H₂O (12 mL). LC/MS (ESI): m/z=567.2 [M+1]⁺. RT=1.927 min

Step-3 The synthesis of Compound 109: The title compound Compound 109 (33 mg mmol, 36% yield) was prepared according to the General Method G with 140-B (112 mg, 0.20 mmol) and TFA (3 mL) in DCM (10 mL).

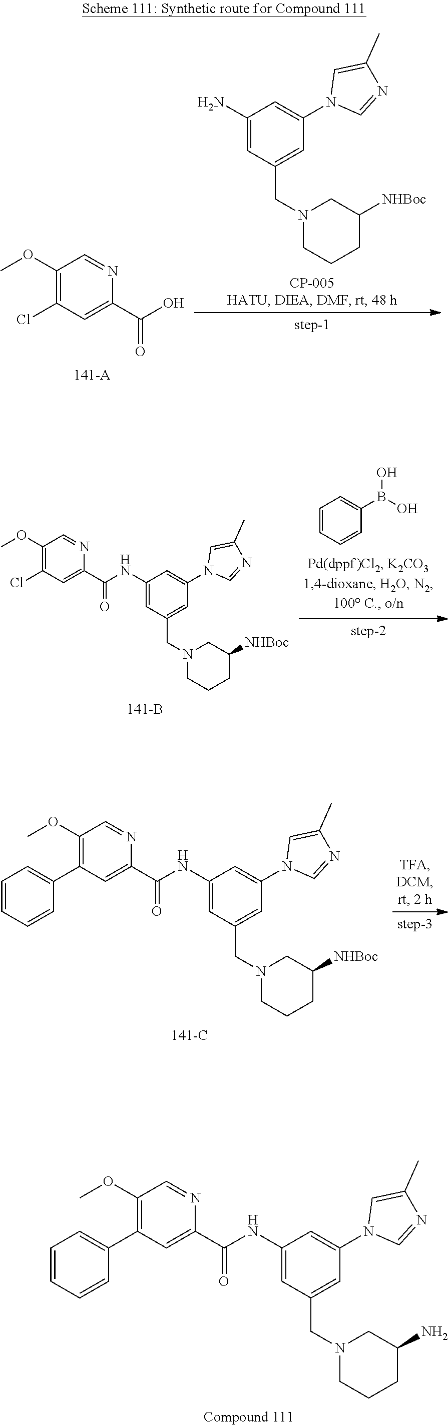 [SMILES: COc1cnc(C(=O)O)cc1Cl ; Cc1cn(-c2cc(N)cc(CN3CCCC(NC(=O)OC(C)(C)C)C3)c2)cn1 ; COc1cnc(C(=O)Nc2cc(CN3CCC[C@H](NC(=O)OC(C)(C)C)C3)cc(-n3cnc(C)c3)c2)cc1Cl ; OB(O)c1ccccc1 ; COc1cnc(C(=O)Nc2cc(CN3CCC[C@H](NC(=O)OC(C)(C)C)C3)cc(-n3cnc(C)c3)c2)cc1-c1ccccc1 ; COc1cnc(C(=O)Nc2cc(CN3CCC[C@H](N)C3)cc(-n3cnc(C)c3)c2)cc1-c1ccccc1]

Step-1 The synthesis of 141-B: To a solution of 141-A (243 mg, 1.298 mmol) and CP-005 (415 mg, 1.08 mmol) in DMF (30 mL) was added HATU (615 mg, 1.62 mmol) and DIEA (421 mg, 3.24 mmol), and the reaction mixture was allowed to stir at room temperature overnight. After consumption of the starting material, the reaction mixture was diluted by water (100 mL) and extracted with EtOAc (3×50 mL). The organic extract was washed by water (4×100 mL) and brine (100 mL) successively, dried over anhydrous Na₂SO₄, filtered and evaporated under reduced pressure to give a residue. The residue was purified by column chromatography on silica gel (eluting with 55% EA in PE) to give 141-B (550 mg, 91% yield) as a white solid. LC/MS (ESI): m/z=555.0 [M+1]⁺. RT=1.984 min

Step-2 The synthesis of 141-C: To a mixture of 141-B (642 mg, 1.16 mmol), phenylboronic acid (212 mg, 1.74 mmol), and K₂CO₃ (480 mg, 3.48 mmol) in 1, 4-dioxane (30 mL) and H₂O (6 mL) was added Pd(dppf)Cl₂ (170 mg, 0.23 mmol), and the reaction mixture was allowed to heat to 100° C. and stir overnight under nitrogen atmosphere. After cooling to room temperature, it was diluted by water (100 mL) and extracted with EtOAc (3×50 mL). The organic extract was washed by brine (100 mL), dried over anhydrous Na₂SO₄ and evaporated under reduced pressure to give a residue. The residue was purified by column chromatography on silica gel (eluting with 30% EA in PE) to give 141-C (400 mg, 82% yield) as a white solid. LC/MS (ESI): m/z=597.1 [M+1]⁺. RT=2.331 min

Step-3 The synthesis of Compound 111: To a solution of 141-C (400 mg, 0.67 mmol) in DCM (15 mL) was added TFA (4 mL), and the reaction mixture was stirred at room temperature for 2 h. After consumption of the starting material, the volatiles were evaporated under reduced pressure to give a residue. The resulted residue was dissolved in DCM (50 mL) and basified by aqueous K₂CO₃ (30 mL). The organic layer was separated, washed by water (50 mL) and brine (50 mL) successively, dried over anhydrous Na₂SO₄, filtered and evaporated under reduced pressure and purified by Prep-HPLC to give Compound 111 (106 mg, 32% yield) as a white solid.

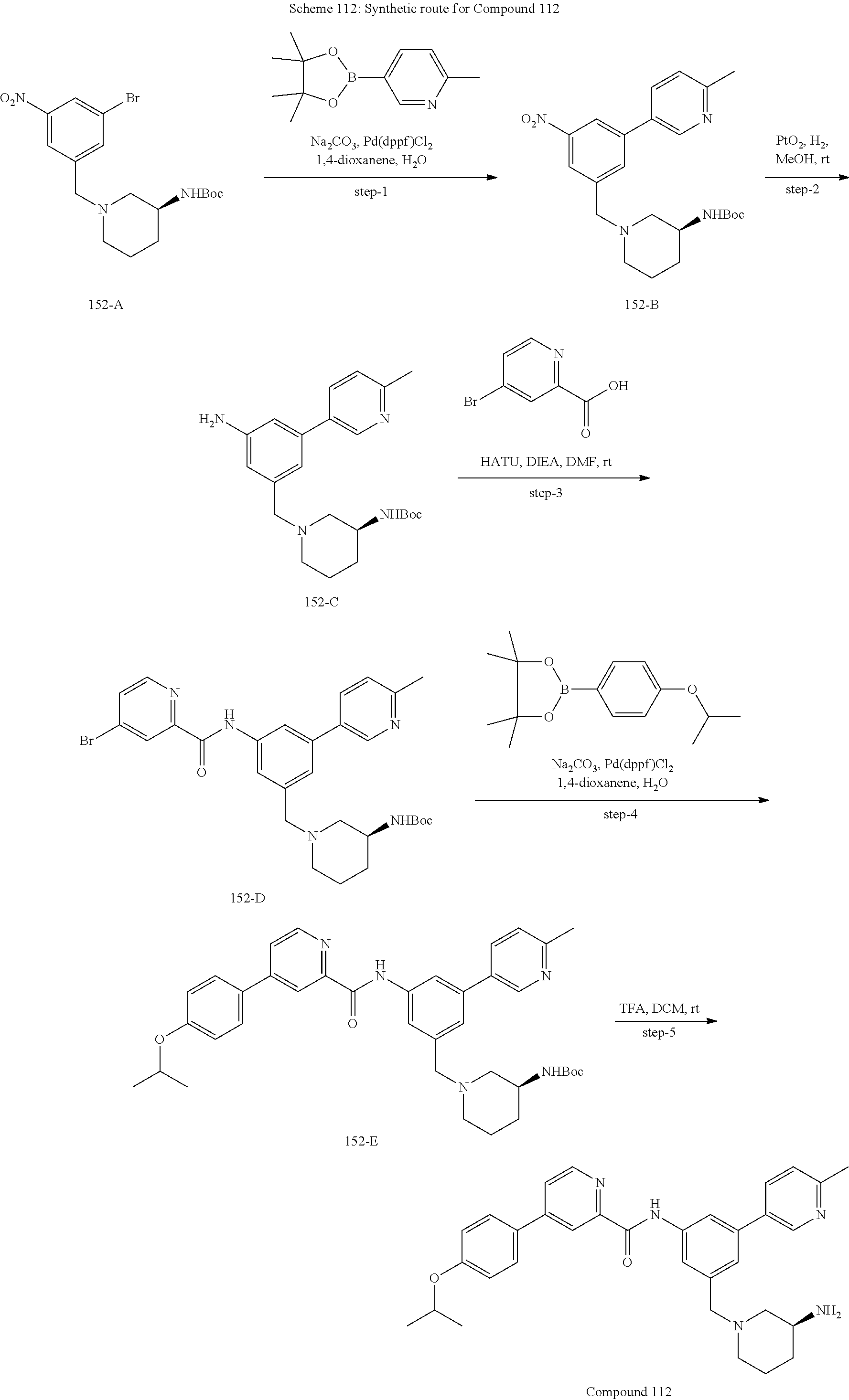

Step-1 The synthesis of 152-B: The title compound 152-B (980 mg, 2.3 mol, 95% yield) was prepared according to the General Method F1 with 152-A (1.0 g, 2.42 mmol), 2-methyl-5-(4,4,5,5-tetramethyl-1,3,2-dioxaborolan-2-yl)pyridine (636 mg, 2.90 mmol), Na₂CO₃ (770 mg, 7.26 mmol) and Pd(dppf)Cl₂ (177 mg, 0.242 mmol) in 1,4-dioxane (60 mL) and H₂O (12 mL).

Step-2 The synthesis of 152-C: The title compound 152-C (800 mg, 4.89 mmol, 88% yield) was prepared according to the General Method D1 with 152-B (980 mg, 2.3 mmol), PtO₂ (80 mg) in MeOH (50 mL).

Step-3 The synthesis of 152-D: The title compound 152-D (245 mg, 0.42 mmol, 55.94% yield) was prepared according to the General Method E with 152-C (300 mg, 0.76 mmol), 6-bromonicotinic acid (182 mg, 0.92 mmol), HATU (433 mg, 1.14 mmol) and DIEA (294 mg, 2.28 mmol) in DMF (15 mL). LC/MS (ESI): m/z=580.0 [M+1]⁺. RT=2.140 min

Step-4 The synthesis of 152-E: The title compound 152-E (152 mg, 0.29 mol, 57% yield) was prepared according to the General Method F1 with 152-D (245 mg, 0.42 mmol), 2-(4-isopropoxyphenyl)-4,4,5,5-tetramethyl-1,3,2-dioxaborolane (132 mg, 0.504 mmol), Na₂CO₃ (134 mg, 1.26 mmol) and Pd(dppf)Cl₂ (31 mg, 0.042 mmol) in 1,4-dioxane (60 mL) and H₂O (12 mL). LC/MS (ESI): m/z=636.2 [M+1]⁺. RT=2.152 min

Step-5 The synthesis of Compound 112: The title compound Compound 112 (60 mg, 39% yield) was prepared according to the General Method G with 152-E (152 mg, 0.29 mmol) and TFA (3 mL) in DCM (10 mL).

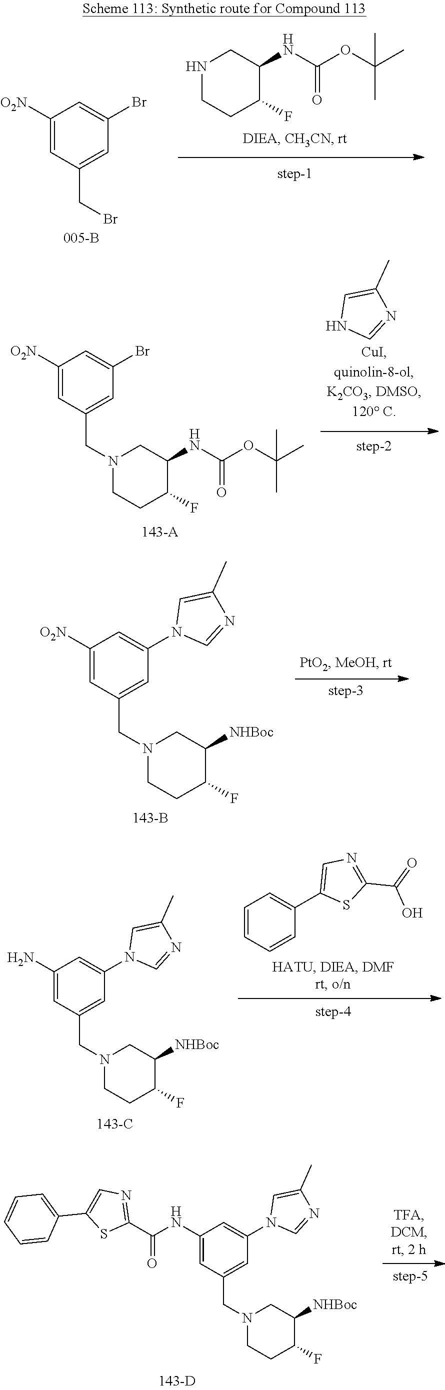

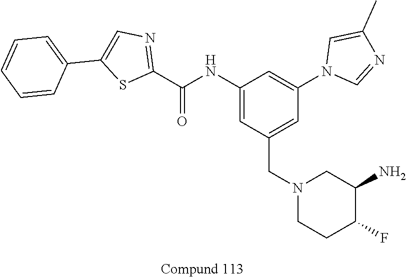 [SMILES: Cc1cn(-c2cc(CN3CC[C@@H](F)[C@H](N)C3)cc(NC(=O)c3ncc(-c4ccccc4)s3)c2)cn1]

Step-1 The synthesis of 143-A: To a mixture of 005-B (1.0 g, 3.43 mmol), tert-butyl ((3R,4R)-4-fluoropiperidin-3-yl)carbamate (500 mg, 2.29 mmol) in CH₃CN (20 mL) was added DIEA (1.2 mL, 6.87 mmol), and the reaction mixture was stirred at room temperature overnight. After consumption of the starting material, it was concentrated to remove CH₃CN, water (50 mL) was added and the mixture was extracted with EtOAc (2×50 mL). The combined organic layers were washed with brine (50 mL), dried over anhydrous Na₂SO₄ and concentrated under reduced pressure to give a residue. The residue was purified by column chromatography on silica gel (eluting with 15% EA in PE) to give 143-A (875 mg, 59% yield) as a white solid. LC/MS (ESI): m/z=432.0 [M+1]⁺. RT=1.91 min

Step-1 The synthesis of 143-B: A suspension of 143-A (432 mg, 1.0 mmol), 4-methyl-1H-imidazole (246 mg, 3.0 mmol), K₂CO₃ (276 mg, 2.0 mmol), CuI (57 mg, 0.3 mmol) and quinolin-8-ol (29 mg, 0.2 mmol) in DMSO (20 mL) was heated at 120° C. overnight under nitrogen atmosphere. After cooling to room temperature, water (50 mL) was added and the mixture was extracted with EtOAc (2×50 mL). The combined organic layers were washed with water (4×100 mL) and brine (50 mL) successively, dried over Na₂SO₄ and concentrated under reduced pressure to give a residue. The residue was purified by column chromatography on silica gel (eluting with EA) to give 143-B (105 mg, 24% yield) as a yellow solid. LC/MS (ESI): m/z=434.0 [M+1]⁺. RT=2.04 min

Step-3 The synthesis of 143-C: To a solution of 143-B (105 mg, 0.24 mmol) in MeOH (20 mL) was added PtO₂ (30 mg), and the reaction mixture was stirred at room temperature for 3 h under hydrogen atmosphere. After consumption of the starting material, the reaction mixture was filtered through a celite pad to remove the solid and the filtrate was evaporated under reduced pressure to give crude 143-C (97 mg, 99% yield) as brown solid. LC/MS (ESI): m/z=404.1 [M+1]⁺. RT=1.80 min

Step-4 The synthesis of 143-D: To a solution of 143-C (97 mg, 0.24 mmol) and 5-phenylthiazole-2-carboxylic acid (60 mg, 0.29 mmol) in DMF (15 mL) was added HATU (138 mg, 0.36 mmol) and DIEA (1 mL), and the reaction mixture was allowed to stir at room temperature overnight. After consumption of the starting material, the reaction mixture was diluted by water (30 mL) and extracted with EtOAc (2×30 mL). The organic extract was washed by water (4×500 mL) and brine (30 mL) successively, dried over anhydrous Na₂SO₄, filtered and evaporated under reduced pressure to give a residue. The residue was purified by Prep-TLC (eluting with 5% MeOH in DCM) to give 143-D (38 mg, 26% yield) as a yellow solid. LC/MS (ESI): m/z=591.2 [M+1]⁺. RT=1.91 min

Step-5 The synthesis of Compound 113: To a solution of 143-D (38 mg, 0.064 mmol) in DCM (10 mL) was added TFA (2.5 mL), and the reaction mixture was stirred at room temperature for 2 h. After consumption of the starting material, the volatiles were evaporated under reduced pressure to give a residue. The resulted residue was dissolved in DCM (20 mL) and neutralized by aqueous K₂CO₃ (20 mL). The organic layer was separated, washed by water (20 mL) and brine (20 mL) successively, dried over anhydrous Na₂SO₄, filtered, evaporated under reduced pressure and purified by Prep-HPLC to give Compound 113 (7 mg, 22% yield) as a white solid.

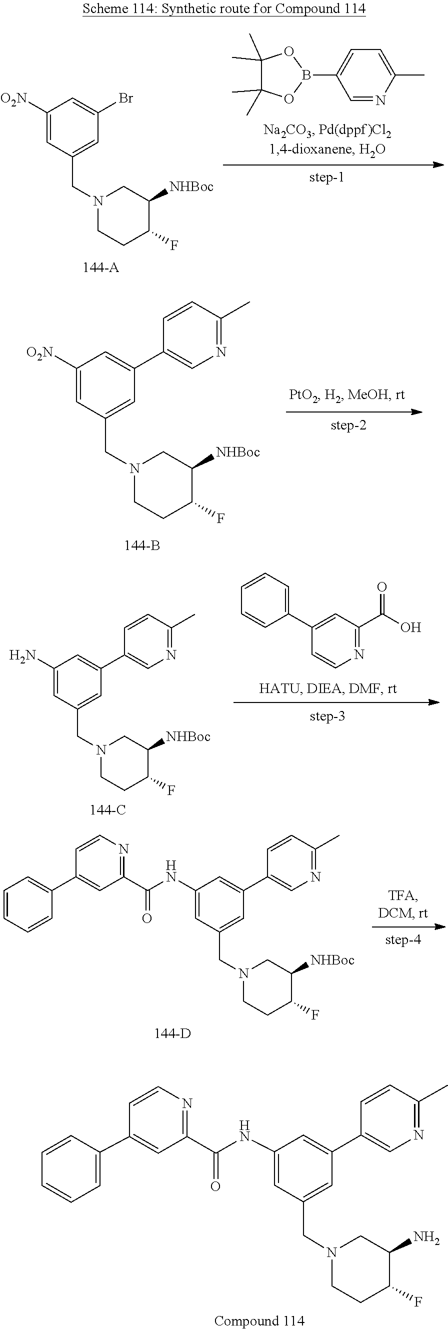

Step-1 The synthesis of 144-B: The title compound 144-B (172 mg, 0.39 mol, 76% yield) was prepared according to the General Method F1 with 144-A (220 mg, 0.51 mmol), 2-methyl-5-(4,4,5,5-tetramethyl-1,3,2-dioxaborolan-2-yl)pyridine (159 mg, 0.62 mmol), Na₂CO₃ (162 mg, 1.53 mmol) and Pd(dppf)Cl₂ (39 mg, 0.051 mmol) in 1,4-dioxane (60 mL) and H₂O (12 mL). LC/MS (ESI): m/z=445.0 [M+1]⁺. RT=2.186 min

Step-2 The synthesis of 144-C: The title compound 144-C (128 mg, 0.30 mmol, 79% yield) was prepared according to the General Method D1 with 144-B (172 mg, 0.39 mol), PtO₂ (20 mg) in MeOH (30 mL). LC/MS (ESI): m/z=415.1 [M+1]⁺. RT=1.926 min

Step-3 The synthesis of 144-D: The title compound 144-D (91 mg, 0.15 mmol, 51% yield) was prepared according to the General Method E with 144-C (128 mg, 0.30 mmol), 111B (66 mg, 0.33 mmol), HATU (171 mg, 0.45 mmol) and DIEA (117 mg, 0.90 mmol) in DMF (15 mL). LC/MS (ESI): m/z=597.9 [M+1]⁺. RT=2.327 min

Step-4 The synthesis of Compound 114: The title compound Compound 114 (62 mg 84% yield) was prepared according to the General Method G with 144-D (90 mg, 0.15 mmol) and TFA (3 mL) in DCM (10 mL).

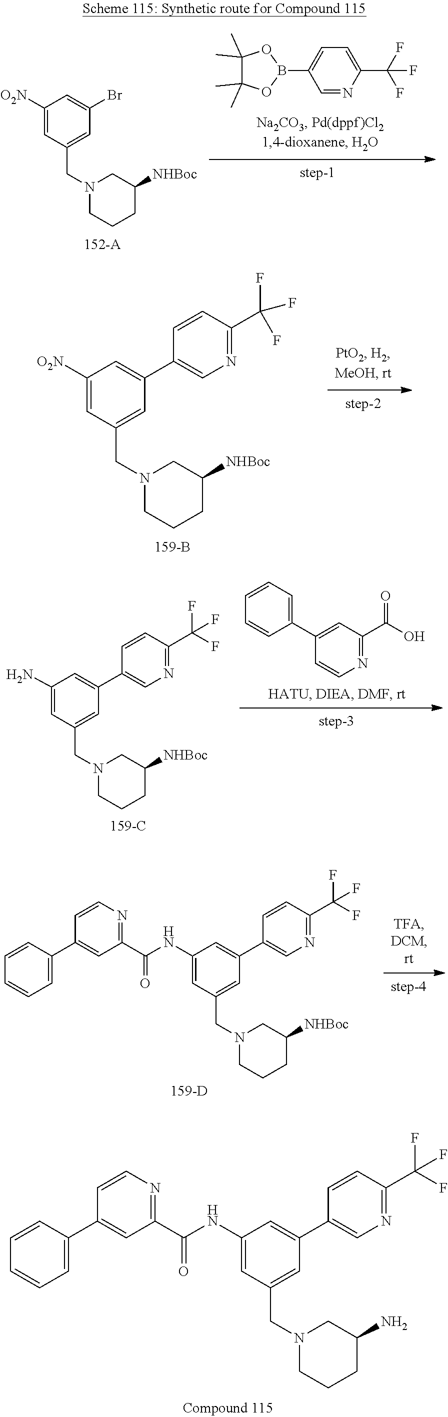

Step-1 The synthesis of 159-B: The title compound 159-B (195 mg, 0.41 mol, 66% yield) was prepared according to the General Method F1 with 152-A (250 mg, 0.61 mmol), 5-(4,4,5,5-tetramethyl-1,3,2-dioxaborolan-2-yl)-2-(trifluoromethyl)pyridine (200 mg, 0.73 mmol), Na₂CO₃ (194 mg, 1.83 mmol) and Pd(dppf)Cl₂ (45 mg, 0.061 mmol) in 1,4-dioxane (60 mL) and H₂O (12 mL). LC/MS (ESI): m/z=481.2 [M+1]⁺. RT=2.156 min

Step-2 The synthesis of 159-C: The title compound 159-C (157 mg, 0.35 mmol, 85% yield) was prepared according to the General Method D1 with 159-B (195 mg, 0.41 mol), PtO₂ (20 mg) in MeOH (30 mL). LC/MS (ESI): m/z=451.2 [M+1]⁺. RT=1.778 min

Step-3 The synthesis of 159-D: The title compound 169-D (100 mg, 0.16 mmol, 43% yield) was prepared according to the General Method E with 159-C (157 mg, 0.35 mmol), 111B (77 mg, 0.39 mmol), HATU (200 mg, 0.53 mmol) and DIEA (151 mg, 1.17 mmol) in DMF (15 mL). LC/MS (ESI): m/z=632.2 [M+1]⁺. RT=2.168 min

Step-4 The synthesis of Compound 115: The title compound Compound 115 (41 mg, 48% yield) was prepared according to the General Method G with 159-D (100 mg, 0.16 mmol) and TFA (3 mL) in DCM (10 mL).

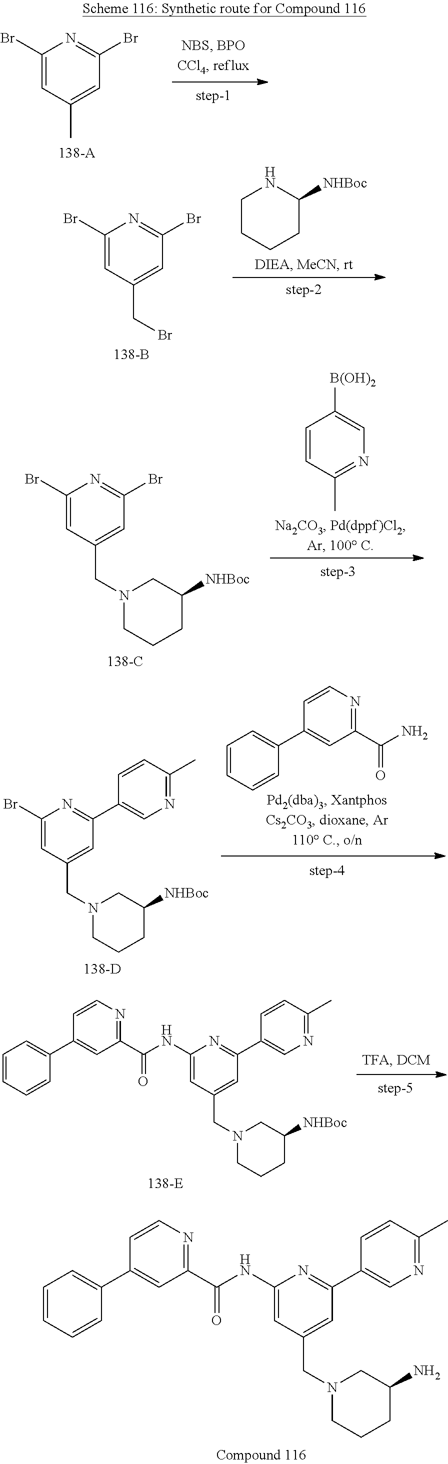

Step-1 The synthesis of 138-B: The title compound 138-B (1.3 g, 3.98 mol, 40% yield) was prepared according to the General Method A with 138-A (2.47 g, 9.96 mmol), NBS (1.95 g, 10.96 mmol) and benzoyl peroxide (235 mg, 0.97 mmol) in CCl₄ (40 mL).

Step-2 The synthesis of 138-C: The title compound 138-C (1.14 g, 3.98 mol, 40% yield) was prepared according to the General Method B1 with 138-B (1.3 g, 3.98 mol), tert-butyl (S)-piperidin-2-ylcarbamate (955 mg, 4.78 mmol), and DIEA (1.54 g, 11.94 mmol) in CH₃CN (30 mL).

Step-3 The synthesis of 138-D: The title compound 138-D (92 mg, 0.2 mol, 30% yield) was prepared according to the General Method F1 with 138-C (300 mg, 0.67 mol), 2-methylpyridin-5-ylboronic acid (110 mg, 0.81 mmol), Na₂CO₃ (213 mg, 2.01 mmol) and Pd(dppf)Cl₂ (49 mg, 0.067 mmol) in 1,4-dioxane (60 mL) and H₂O (12 mL).

Step-4 The synthesis of 138-E: A mixture of 138-D (92 mg, 0.2 mol), 4-phenylpicolinamide (48 mg, 0.24 mmol), Cs₂CO₃ (196 mg, 0.6 mmol), Xantphos (35 mg, 0.06 mmol) and Pd₂(dba)₃ (27 mg, 0.03 mmol) in 1,4-dioxane (60 mL) was allowed to heat at 100° C. and stirred overnight under argon. After cooling to room temperature, water (50 mL) was added and the mixture was extracted with EtOAc (50 mL×2). The combined organic layers were washed with brine (50 mL), dried over Na₂SO₄ and concentrated. The residue was purified by silica gel column chromatography (eluting with 60% EA in PE) to give 138-E (40 mg, 0.069 mol, 34% yield) as a yellow solid. LC/MS (ESI): m/z=579.2 [M+1]⁺. RT=2.135 min

Step-5 The synthesis of Compound 116: The title compound Compoound 116 (21 mg, 63.6% yield) was prepared according to the General Method G with 138-E (40 mg, 0.069 mmol) and TFA (3 mL) in DCM (10 mL).

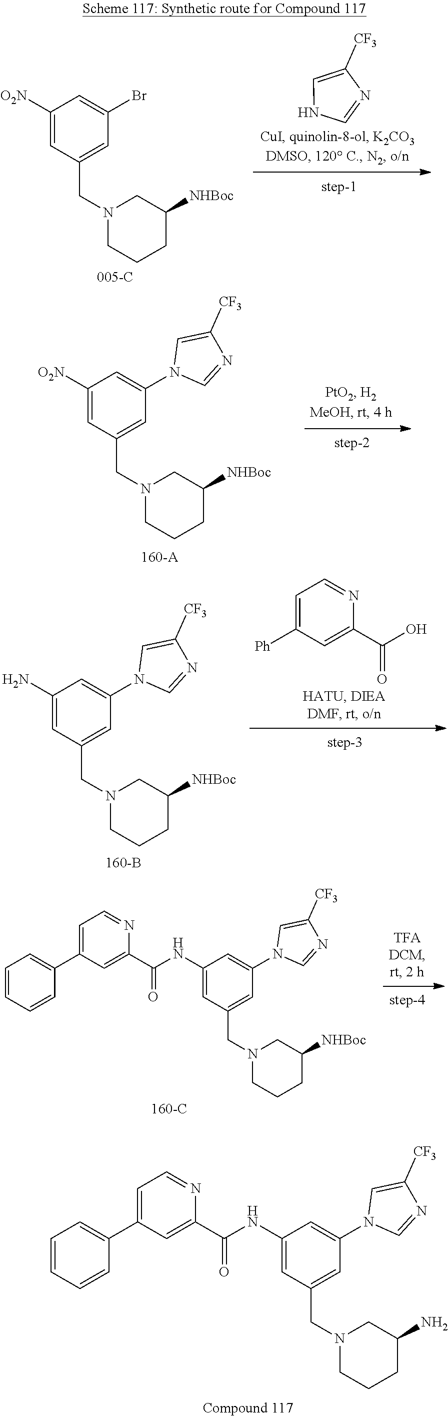

Step-1 The synthesis of 160-A: A suspension of 005-C (300 mg, 0.725 mmol), 4-(trifluoromethyl)-1H-imidazole (295 mg, 2.17 mmol), K₂CO₃ (300 mg, 2.17 mmol), CuI (41 mg, 0.22 mmol) and quinolin-8-ol (21 mg, 0.145 mmol) in DMSO (30 mL) was heated at 120° C. overnight under nitrogen atmosphere. After cooling to room temperature, water (100 mL) was added and the mixture was extracted with EtOAc (2×100 mL). The combined organic layers were washed with water (4×100 mL) and brine (100 mL) successively, dried over Na₂SO₄ and concentrated under reduced pressure to give a residue. The residue was purified by column chromatography on silica gel (eluting with 3% MeOH in DCM) to give 160-A (105 mg, 30% yield) as a yellow solid. LC/MS (ESI): m/z=470.2 [M+1]⁺. RT=1.828 min

Step-2 The synthesis of 160-B: To a solution of 160-A (105 mg, 0.22 mmol) in MeOH (15 mL) was added PtO₂ (20 mg), and the reaction mixture was stirred at room temperature for 12 hours under hydrogen atmosphere. After consumption of the starting material, the reaction mixture was filtered through celite to remove the solid and the filtrate was evaporated under reduced pressure to give crude 160-B (80 mg, 83% yield) as black solid. LC/MS (ESI): m/z=440.2 [M+1]⁺. RT=1.868 min

Step-3 The synthesis of 160-C: To a solution of 160-B (80 mg, 0.18 mmol) and 4-phenylpicolinic acid (36.3 mg, 0.18 mmol) in DMF (20 mL) was added HATU (104 mg, 0.27 mmol) and DIEA (80 mg, 0.55 mmol), and the reaction mixture was allowed to stir at room temperature overnight. After consumption of the starting material, the reaction mixture was diluted by water (100 mL) and extracted with EtOAc (2×50 mL). The organic extract was washed by water (4×100 mL) and brine (100 mL) successively, dried over anhydrous Na₂SO₄, filtered and evaporated under reduced pressure to give a residue. The residue was purified by column chromatography on silica gel (eluting with 3% MeOH in DCM) to give 160-C (65 mg, 58% yield) as a yellow solid. LC/MS (ESI): m/z=621.2 [M+1]⁺. RT=2.076 min

Step-4 The synthesis of Compound 117: To a solution of 160-C (65 mg, 0.1 mmol) in DCM (10 mL) was added TFA (3 mL), and the reaction mixture was stirred at room temperature for 2 h. After consumption of the starting material, the volatiles were evaporated under reduced pressure to give a residue. The resulted residue was dissolved in DCM (50 mL) and basified by aqueous K₂CO₃ (30 mL). The organic layer was separated, washed by water (50 mL) and brine (50 mL) successively, dried over anhydrous Na₂SO₄, filtered and evaporated under reduced pressure. The residue was purified by pre-HPLC to give Compound 117 (11 mg, 20% yield) as a white solid.

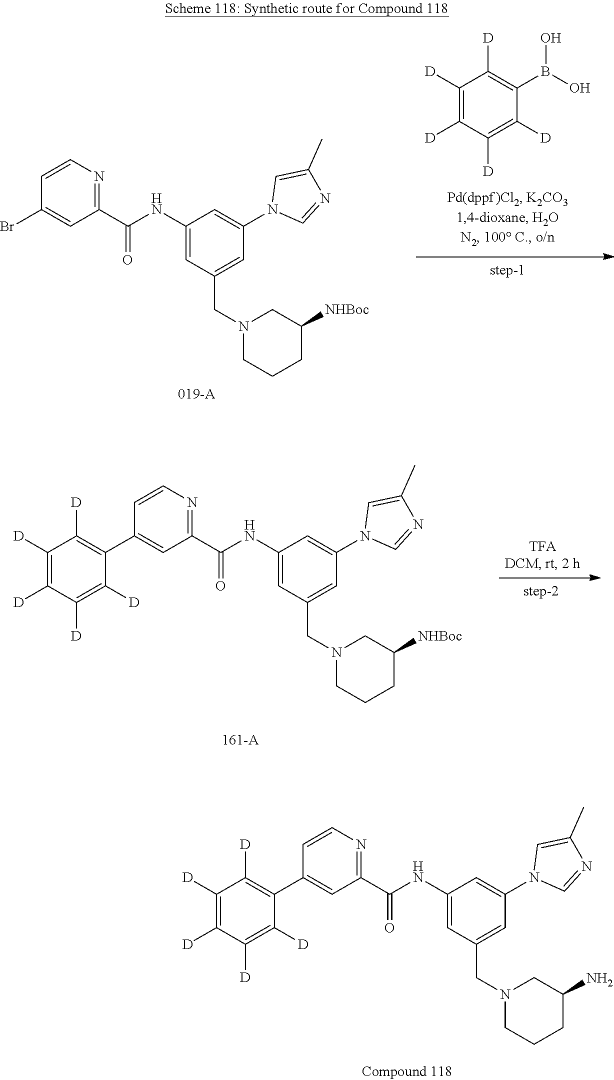

Step-1 The synthesis of 161-A: To a mixture of 019-A (250 mg, 0.44 mmol), (pentadeuterophenyl)boronic acid (67 mg, 0.53 mmol), and K₂CO₃ (182 mg, 1.32 mmol) in 1, 4-dioxane (30 mL) and H₂O (6 mL) was added Pd(dppf)Cl₂ (35 mg, 0.04 mmol), and the reaction mixture was allowed to heat at reflux and stir overnight under nitrogen atmosphere. After cooling to room temperature, it was diluted by water (50 mL) and extracted with EtOAc (2×40 mL). The organic extract was washed by brine (50 mL), dried over anhydrous Na₂SO₄ and evaporated under reduced pressure to give a residue. The residue was purified by column chromatography on silica gel (eluting with 3% MeOH in DCM) to give 161-A (110 mg, 44% yield) as gray solid. LC/MS (ESI): m/z=572.3 [M+1]⁺. RT=1.932 min

Step-2 The synthesis of Compound 118: To a solution of 161-A (110 mg, 0.19 mmol) in DCM (10 mL) was added TFA (3 mL), and the reaction mixture was stirred at room temperature for 2 h. After consumption of the starting material, the volatiles were evaporated under reduced pressure to give a residue. The resulted residue was dissolved in DCM (50 mL) and basified by aqueous K₂CO₃ (20 mL). The organic layer was separated, washed by water (50 mL) and brine (50 mL) successively, dried over anhydrous Na₂SO₄, filtered and evaporated under reduced pressure, and purified by pre-HPLC to give Compound 118 (65 mg, 73% yield) as a white solid.

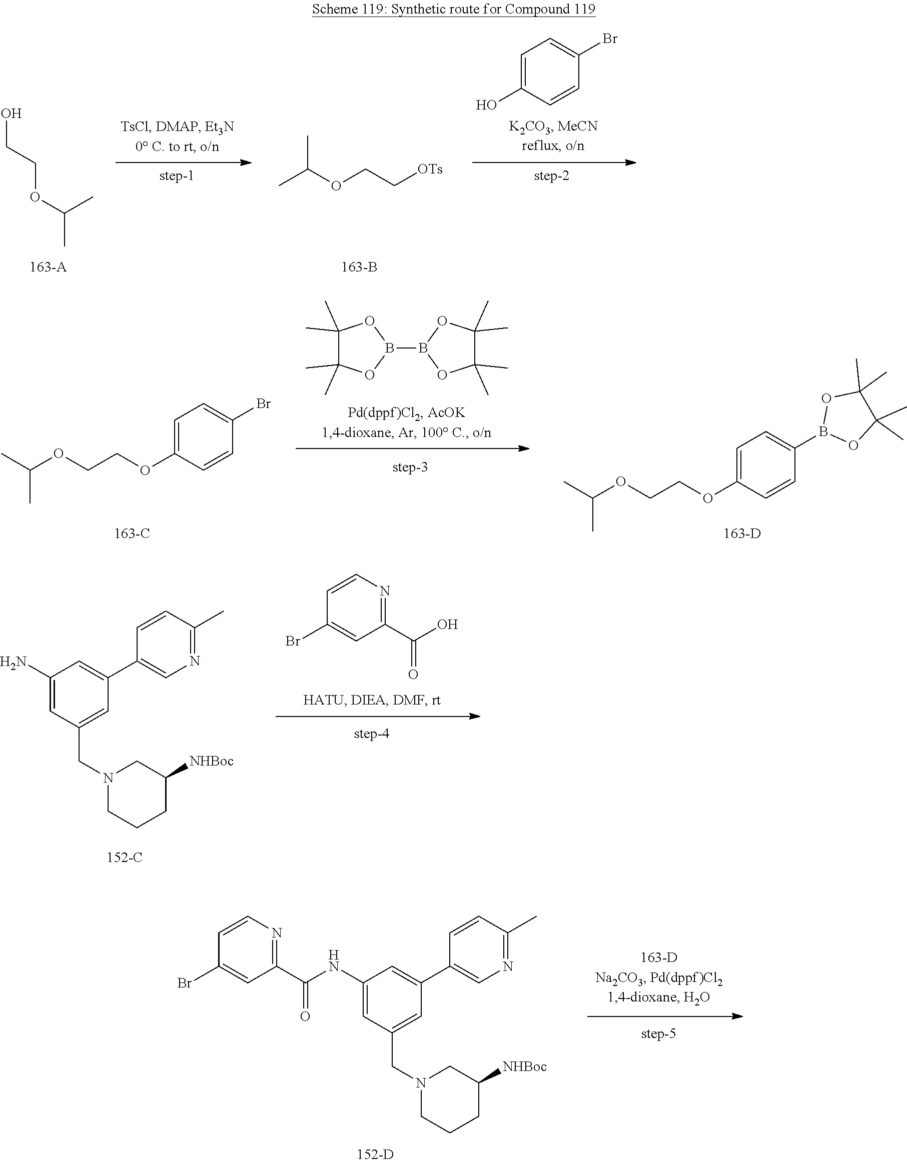

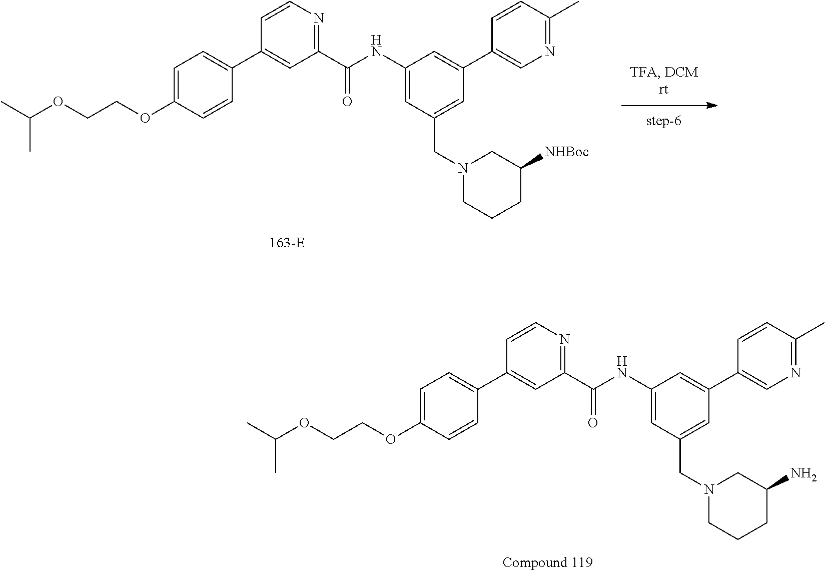

Step-1 The synthesis of 163-B: A mixture of 163-A (2.0 g, 19.23 mmol), TsCl (4.41 g, 23 mmol), DMAP (235 mg, 1.92 mmol), Et₃N (3.89 g, 38.46 mmol) in DCM (100 mL) was stirred at room temperature under nitrogen atmosphere overnight. After consumption of the starting material, water (30 mL) was added and the mixture was extracted with EtOAc (2×30 mL). The combined organic layers were washed with brine (30 mL), dried over anhydrous Na₂SO₄ and concentrated under reduced pressure to give a residue. The residue was purified by column chromatography on silica gel (eluting with 8% EA in PE) to give 163-B (3.8 g, 76% yield) as a yellow solid. LC/MS (ESI): m/z=276.2 [M+18]⁺. RT=1.910 min

Step-2 The synthesis of 163-C: The title compound 163-C (1.9 g, 3.98 mol, 95% yield) was prepared according to the General Method B2 with 163-B (2.0 g, 7.75 mmol), 4-bromophenol (2.0 g, 11.63 mmol) and K₂CO₃ (3.2 g, 23.25 mmol) in CH₃CN (30 mL). LC/MS (ESI): m/z=276.2 [M+18]⁺. RT=2.11 min

Step-3 The synthesis of 163-D: The title compound 163-D (1.8 g, 5.89 mol, 80% yield) was prepared according to the General Method I with 163-C (1.9 g, 7.36 mol), bis(pinacolato)diborane (3.74 g, 14.72 mmol), AcOK (2.16 g, 22.08 mmol) and Pd(dppf)Cl₂ (539 mg, 0.736 mmol) in 1,4-dioxane (100 mL) and H₂O (20 mL). LC/MS (ESI): m/z=324.2 [M+18]⁺. RT=1.983 min

Step-4 The synthesis of 152-D: The title compound 152-D (370 mg, 0.64 mmol, 88% yield) was prepared according to the General Method E with 152-C (290 mg, 0.73 mmol), 4-bromopicolinic acid (178 mg, 0.88 mmol), HATU (362 mg, 0.95 mmol) and DIEA (283 mg, 2.19 mmol) in DMF (15 mL).

Step-5 The synthesis of 163-E: The title compound 163-E (80 mg, 0.12 mol, 34% yield) was prepared according to the General Method F1 with 152-D (200 mg, 0.35 mol), 163-D (128 mg, 0.42 mmol), Na₂CO₃ (111 mg, 1.05 mmol) and Pd(dppf)Cl₂ (26 mg, 0.035 mmol) in 1,4-dioxane (30 mL) and H₂O (6 mL). LC/MS (ESI): m/z=680.2 [M+1]⁺. RT=2.121 min

Step-6 The synthesis of Compound 119: The title compound Compound 119 (36 mg, 52% yield) was prepared according to the General Method G with 163-E (80 mg, 0.12 mmol) and TFA (3 mL) in DCM (10 mL).

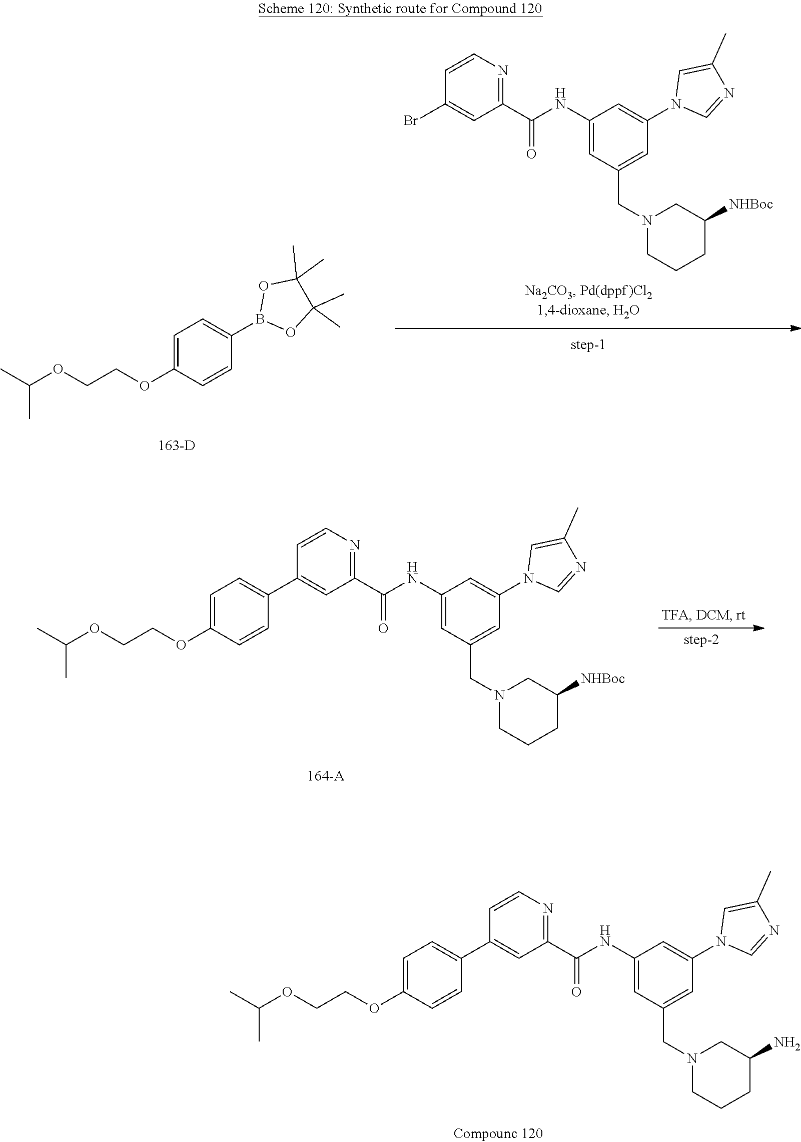 [SMILES: CC(C)OCCOc1ccc(B2OC(C)(C)C(C)(C)O2)cc1 ; Cc1cn(-c2cc(CN3CCC[C@H](NC(=O)OC(C)(C)C)C3)cc(NC(=O)c3cc(Br)ccn3)c2)cn1 ; Cc1cn(-c2cc(CN3CCC[C@H](NC(=O)OC(C)(C)C)C3)cc(NC(=O)c3cc(-c4ccc(OCCOC(C)C)cc4)ccn3)c2)cn1 ; Cc1cn(-c2cc(CN3CCC[C@H](N)C3)cc(NC(=O)c3cc(-c4ccc(OCCOC(C)C)cc4)ccn3)c2)cn1]

Step-1 The synthesis of 164-A: The title compound 164-A (70 mg, 0.10 mmol, 20% yield) was prepared according to the General Method F1 with 163-D (160 mg, 0.52 mmol), 019-A (250 mg, 0.44 mmol), Na₂CO₃ (140 mg, 1.32 mmol) and Pd(dppf)Cl₂ (32 mg, 0.044 mmol) in 1,4-dioxane (30 mL) and H₂O (6 mL). LC/MS (ESI): m/z=669.3 [M+1]⁺. RT=2.006 min

Step-2 The synthesis of Compound 120: The title compound Compound 120 (41 mg, 72% yield) was prepared according to the General Method G with 164-A (70 mg, 0.10 mmol) and TFA (3 mL) in DCM (10 mL).

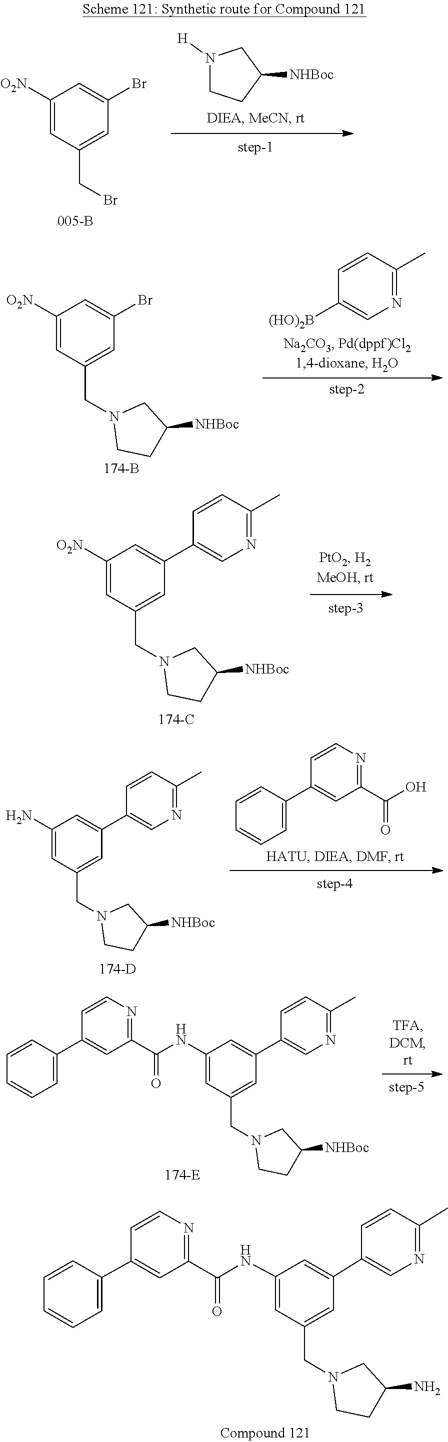

Step-1 The synthesis of 174-B: The title compound 174-B (400 mg, 1 mmol, 49% yield) was prepared according to the General Method B1 with 005-B (600 mg, 2.03 mmol), tert-butyl (S)-pyrrolidin-3-ylcarbamate (415 mg, 2.23 mmol) and K₂CO₃ (3.2 g, 23.25 mmol) in CH₃CN (30 mL). LC/MS (ESI): m/z=400.0 [M+1]⁺. RT=1.867 min

Step-2 The synthesis of 174-C: The title compound 174-C (200 mg, 0.48 mmol, 48% yield) was prepared according to the General Method F1 with 174-B (400 mg, 1 mmol), 2-methylpyridin-5-ylboronic acid (164 mg, 1.2 mmol), Na₂CO₃ (309 mg, 3 mmol) and Pd(dppf)Cl₂ (146 mg, 0.2 mmol) in 1,4-dioxane (30 mL) and H₂O (6 mL). LC/MS (ESI): m/z=413.2 [M+1]⁺. RT=1.713 min

Step-3 The synthesis of 174-D: The title compound 174-D (175 mg, 0.46 mmol, 95% yield) was prepared according to the General Method D1 with 174-C (200 mg, 0.48 mmol), PtO₂ (20 mg) in MeOH (50 mL). LC/MS (ESI): m/z=383.2 [M+1]⁺. RT=1.667 min

Step-4 The synthesis of 174-E: The title compound 174-E (145 mg, 0.26 mmol, 56% yield) was prepared according to the General Method E with 174-D (175 mg, 0.46 mmol), 111B (100 mg, 0.51 mmol), HATU (227 mg, 0.598 mmol) and DIEA (178 mg, 1.38 mmol) in DMF (15 mL). LC/MS (ESI): m/z=564.0 [M+1]⁺. RT=2.129 min

Step-5 The synthesis of Compound 121: The title compound Compound 121 (60 mg, 50% yield) was prepared according to the General Method G with 174-E (145 mg, 0.26 mmol) and TFA (3 mL) in DCM (10 mL).

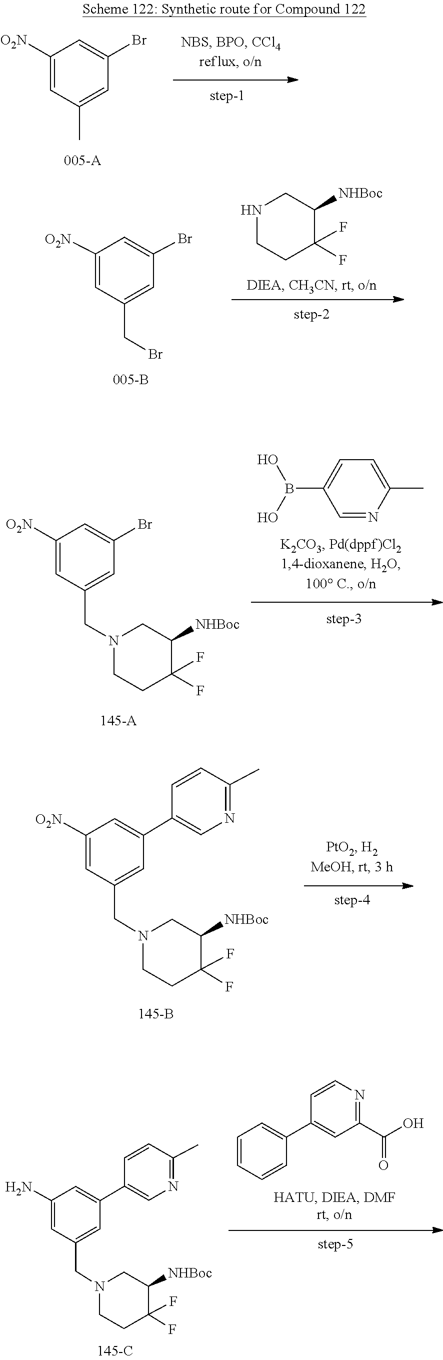

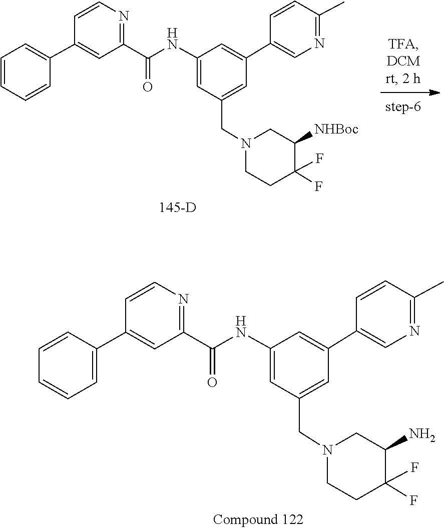 [SMILES: Cc1ccc(-c2cc(CN3CCC(F)(F)[C@H](NC(=O)OC(C)(C)C)C3)cc(NC(=O)c3cc(-c4ccccc4)ccn3)c2)cn1 ; Cc1ccc(-c2cc(CN3CCC(F)(F)[C@H](N)C3)cc(NC(=O)c3cc(-c4ccccc4)ccn3)c2)cn1]

Step-1 The synthesis of 005-B: To a mixture of 005-A (25.0 g, 115.7 mmol) and NBS (21.6 g, 121.5 mmol) in CCl₄ (250 mL) was added benzoyl peroxide (2.80 g, 11.6 mmol), and the reaction mixture was heated to reflux and stirred overnight. After cooling to room temperature, the precipitate was removed by filtration and the filtrate was evaporated under reduced pressure to give a residue. The residue was purified by column chromatography on silica gel (eluting with 5% EA in PE) to give 005-B (27.0 g, 80% yield) as a yellow solid.

Step-2 The synthesis of 145-A: To a mixture of 005-B (5.62 g, 19.05 mmol), tert-butyl (3R)-(4,4-difluoropiperidin-3-yl)carbamate (4.50 g, 19.05 mmol) in CH₃CN (100 mL) was added DIEA (9.85 g, 76.20 mmol), and the reaction mixture was stirred at room temperature overnight. After consumption of the starting material, it was concentrated to remove CH₃CN, water (100 mL) was added and the mixture was extracted with EtOAc (2×100 mL). The combined organic layers were washed with brine (100 mL), dried over anhydrous Na₂SO₄ and concentrated under reduced pressure to give a residue. The residue was purified by column chromatography on silica gel (eluting with 9% EA in PE) to give 145-A (7.10 g, 83% yield) as a yellow solid. LC/MS (ESI): m/z=449.9 [M+1]⁺. RT=2.38 min

Step-3 The synthesis of 145-B: To a solution of 145-A (1.40 g, 3.11 mmol) and (2-methylpyridin-5-yl)boronic acid (510 mg, 3.73 mmol) and K₂CO₃ (1.29 g, 9.33 mmol) in 1, 4-dioxane (50 mL) and H₂O (10 mL) was added Pd(dppf)Cl₂ (227 mg, 0.31 mmol), and the reaction mixture was allowed to heat to 100° C. and stir overnight under argon atmosphere. After cooling to room temperature, it was diluted by water (50 mL) and extracted with EtOAc (2×50 mL). The organic extract was washed by brine (50 mL), dried over anhydrous Na₂SO₄ and evaporated under reduced pressure to give a residue. The residue was purified by column chromatography on silica gel (eluting with DCM:MeOH=30:1) to give 145-B (1.3 g, 90% yield) as a white solid. LC/MS (ESI): m/z=463.1 [M+1]⁺. RT=2.00 min

Step-4 The synthesis of 145-C: To a solution of 145-B (1.3 g, 2.81 mmol) in MeOH (100 mL) was added PtO₂ (100 mg), and the reaction mixture was stirred at room temperature for 3 h under hydrogen atmosphere. After consumption of the starting material, the reaction mixture was filtered through a celite pad to remove the solid and the filtrate was evaporated under reduced pressure to give crude 145-C (1.1 g, 91% yield) as gray solid. LC/MS (ESI): m/z=433.1 [M+1]⁺. RT=1.82 min

Step-5 The synthesis of 145-D: To a solution of 145-C (300 mg, 0.69 mmol) and 4-phenylpicolinic acid (208 mg, 1.04 mmol) in DMF (30 mL) was added HATU (570 mg, 1.04 mmol) and DIEA (270 mg, 2.08 mmol), and the reaction mixture was allowed to stir at room temperature overnight. After consumption of the starting material, the reaction mixture was diluted by water (50 mL) and extracted with EtOAc (2×50 mL). The organic extract was washed by water (4×100 mL) and brine (50 mL) successively, dried over anhydrous Na₂SO₄, filtered and evaporated under reduced pressure to give a residue. The residue was purified by column chromatography on silica gel (eluting with 50% EA in PE) to give 145-D (160 mg, 38% yield) as a light-yellow solid. LC/MS (ESI): m/z=614.0 [M+1]⁺. RT=2.47 min

Step-7 The synthesis of Compound 122: To a solution of 145-D (160 mg, 0.26 mmol) in DCM (15 mL) was added TFA (3 mL), and the reaction mixture was stirred at room temperature for 2 h. After consumption of the starting material, the volatiles were evaporated under reduced pressure to give a residue. The resulted residue was dissolved in DCM (30 mL) and basified by aqueous K₂CO₃ (30 mL). The organic layer was separated, washed by water (30 mL) and brine (30 mL) successively, dried over anhydrous Na₂SO₄, filtered, evaporated under reduced pressure and purified by Prep-HPLC to give Compound 122 (81 mg, 61% yield) as a white solid.

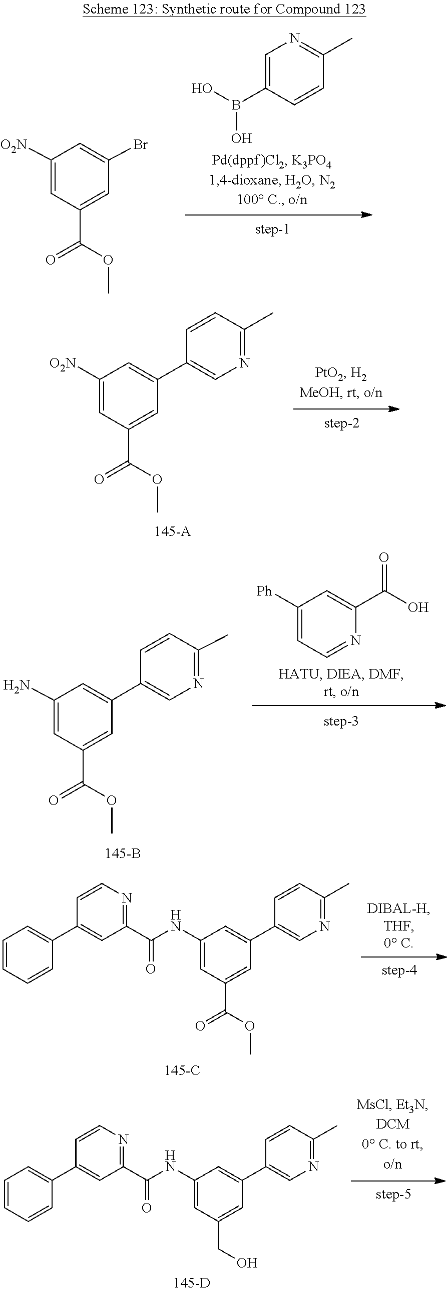

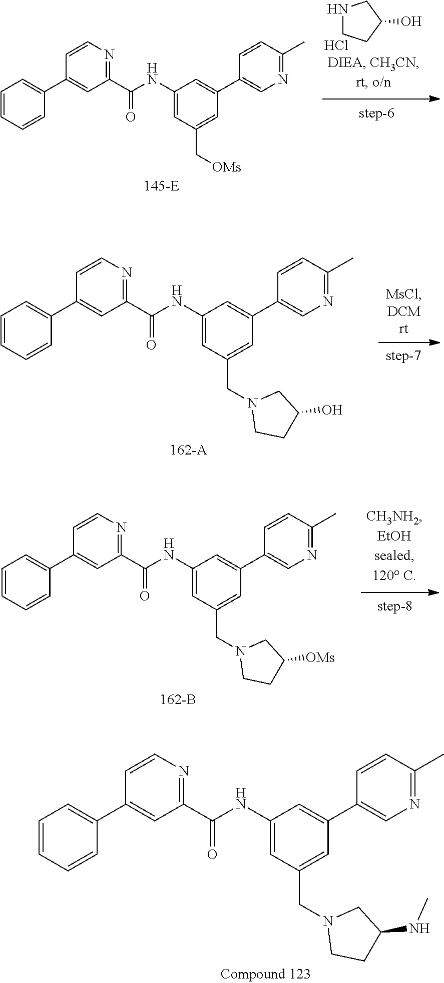

Step-1 The synthesis of 145-A: To a mixture of methyl 3-bromo-5-nitrobenzoate (4.0 g, 15.4 mmol), 2-methylpyridin-5-ylboronic acid (2.1 g, 15.4 mmol), and K₃PO₃ (9.8 g, 46.2 mmol) in 1, 4-dioxane (100 mL) and H₂O (20 mL) was added Pd(dppf)Cl₂ (1.12 g, 1.54 mmol), and the reaction mixture was allowed to heat at reflux and stir overnight under nitrogen atmosphere. After cooling to room temperature, it was diluted by water (200 mL) and extracted with EtOAc (2×200 mL). The organic extract was washed by brine (200 mL), dried over anhydrous Na₂SO₄ and evaporated under reduced pressure to give a residue. The residue was purified by column chromatography on silica gel (eluting with 3% MeOH in DCM) to give 145-A (1.5 g, 35% yield) as a white solid. LC/MS (ESI): m/z=273.1 [M+1]⁺. RT=1.565 min

Step-2 The synthesis of 145-B: To a solution of 145-A (1.0 g, 3.67 mmol) in MeOH (100 mL) was added PtO₂ (100 mg), and the reaction mixture was stirred at room temperature for 12 hours under hydrogen atmosphere. After consumption of the starting material, the reaction mixture was filtered through celite to remove the solid and the filtrate was evaporated under reduced pressure to give crude 145-B (550 g, 62% yield) as a yellow oil. LC/MS (ESI): m/z=243.2 [M+1]⁺. RT=1.547 min

Step-3 The synthesis of 145-C: To a solution of 145-B (0.55 g, 2.27 mmol) and 4-phenylpicolinic acid (0.45 g, 2.27 mmol) in DMF (30 mL) was added HATU (1.3 g, 3.41 mmol) and DIEA (0.88 g, 6.81 mmol), and the reaction mixture was allowed to stir at room temperature overnight. After consumption of the starting material, the reaction mixture was diluted by water (100 mL) and extracted with EtOAc (2×100 mL). The organic extract was washed by water (4×200 mL) and brine (200 mL) successively, dried over anhydrous Na₂SO₄, filtered and evaporated under reduced pressure to give a residue. The residue was purified by column chromatography on silica gel (eluting with 3% MeOH in DCM) to give 145-C (0.94 g, 95% yield) as a yellow solid. LC/MS (ESI): m/z=424.2 [M+1]⁺. RT=1.880 min

Step-4 The synthesis of 145-D: To a solution of 145-C (1.0 g, 2.36 mmol) in THF (20 mL) was added DIBAL-H of toluene solution (1 N, 8 mL), and the reaction mixture was stirred at 0° C. for 2 hours under argon atmosphere. After consumption of the starting material, the reaction mixture was quenched by 10% NaOH aqueous solution (50 mL) slowly at 0° C. The mixture was evaporated in vacuo to remove the volatiles and extracted with EtOAc (3×50 mL). The combined organic extract was dried over anhydrous Na₂SO₄, filtered and evaporated under reduced pressure to give 145-D (0.34 g, 37% yield) as grey solid. LC/MS (ESI): m/z=396.2 [M+1]⁺. RT=1.828 min

Step-5 The synthesis of 145-E: To a solution of 145-D (340 mg, 0.86 mmol) in dry DCM (20 mL) was added Et₃N (260 mg, 2.60 mmol) and MsCl (225 mg, 1.29 mmol) at 0° C., and the reaction mixture was allowed to stir at room temperature for 2 hours. After consumption of the starting material, the reaction mixture was evaporated under reduced pressure to remove the volatiles. The residue was diluted by water (100 mL) and extracted with EtOAc (2×40 mL). The organic extract was washed by water (50 mL) and brine (50 mL) successively, dried over anhydrous Na₂SO₄, filtered and evaporated under reduced pressure to give 145-E (250 mg, 61% yield) as grey solid. LC/MS (ESI): m/z=474.2 [M+1]⁺. RT=1.755 min

Step-6 The synthesis of 162-A: To a mixture of 145-E (130 mg, 0.27 mmol), (R)-pyrrolidin-3-ol hydrochloride (40 mg, 0.33 mmol) in CH₃CN (20 mL) was added DIEA (70 mg, 0.54 mmol), and the reaction mixture was stirred at room temperature overnight. After consumption of the starting material, water (50 mL) was added and the mixture was extracted with EtOAc (2×50 mL). The combined organic layers were washed with brine (50 mL), dried over anhydrous Na₂SO₄ and concentrated under reduced pressure to give a residue. The residue was purified by column chromatography on silica gel (eluting with 3% MeOH in DCM) to give 162-A (93 mg, 53% yield) as a yellow oil. LC/MS (ESI): m/z=465.1 [M+1]⁺. RT=2.006 min

Step-7 The synthesis of 162-B: To a solution of 162-A (125 mg, 0.27 mmol) in dry DCM (20 mL) was added Et₃N (82 mg, 0.81 mmol) and MsCl (72 mg, 0.41 mmol), and the reaction mixture was allowed to stir at room temperature for 2 hours. After consumption of the starting material, the reaction mixture was evaporated under reduced pressure, diluted by water (50 mL) and extracted with EtOAc (2×40 mL). The organic extract was washed by water (50 mL) and brine (50 mL) successively, dried over anhydrous Na₂SO₄, filtered and evaporated under reduced pressure to give crude 162-B (100 mg, 68% yield) as a yellow solid. LC/MS (ESI): m/z=543.0 [M+1]⁺. RT=1.965 min

Step-8 The synthesis of Compound 123: A mixture of crude 162-B (50 mg, 0.09 mmol) in a solution of CH₃NH₂ in EtOH (5 mL) was stirred at 120° C. in sealed tube overnight. After consumption of the starting material, water (50 mL) was added and the mixture was extracted with EtOAc (2×50 mL). The combined organic layers were washed with brine (50 mL), dried over anhydrous Na₂SO₄ and concentrated under reduced pressure to give a residue. The residue was purified by column chromatography on silica gel (eluting with 3% MeOH in DCM) to give Compound 123 (3.3 mg, 10% yield) as a white solid.

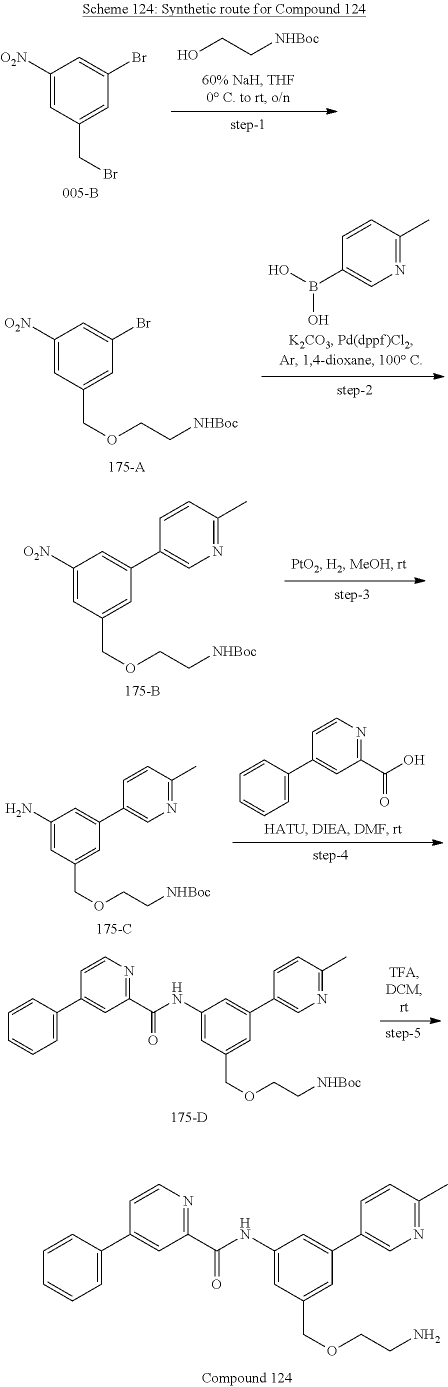

Step-1 The synthesis of 175-A: To a solution of tert-butyl (2-hydroxyethyl)carbamate (483 mg, 3.0 mmol) in dry THF (30 mL) was added 60% NaH (320 mg, 8.0 mmol) slowly at 0° C., after stirring for 30 min, 005-B (590 mg, 2.0 mmol) was added, and the reaction mixture was allowed to stirred at room temperature overnight. After consumption of the starting material, it was quenched by water (30 mL) slowly at 0° C., concentrated to remove THF, and extracted with EtOAc (2×30 mL). The combined organic layers were washed with brine (50 mL), dried over anhydrous Na₂SO₄ and concentrated under reduced pressure to give a residue. The residue was purified by column chromatography on silica gel (eluting with 15˜20% EA in PE) to give 175-A (190 mg, 25% yield) as brown gel. LC/MS (ESI): m/z=318.9 [M−56]⁺. RT=2.23 min

Step-2 The synthesis of 175-B: To a solution of 175-A (590 mg, 1.57 mmol) and (2-methylpyridin-5-yl)boronic acid (260 mg, 1.89 mmol) and K₂CO₃ (650 mg, 4.72 mmol) in 1, 4-dioxane (30 mL) and H₂O (6 mL) was added Pd(dppf)Cl₂ (128 mg, 0.16 mmol), and the reaction mixture was allowed to heat to 100° C. and stir overnight under argon atmosphere. After cooling to room temperature, it was diluted by water (50 mL) and extracted with EtOAc (2×50 mL). The organic extract was washed by brine (50 mL), dried over anhydrous Na₂SO₄ and evaporated under reduced pressure to give a residue. The residue was purified by column chromatography on silica gel (eluting with EA:PE=3:2) to give 175-B (390 mg, 64% yield) as a yellow solid. LC/MS (ESI): m/z=388.2 [M+1]⁺. RT=1.67 min

Step-3 The synthesis of 145-C: To a solution of 175-B (390 mg, 1.0 mmol) in MeOH (30 mL) was added PtO₂ (40 mg), and the reaction mixture was stirred at room temperature for 2 h under hydrogen atmosphere. After consumption of the starting material, the reaction mixture was filtered through a celite pad to remove the solid and the filtrate was evaporated under reduced pressure to give crude 175-C (360 mg, 99% yield) as brown gel. LC/MS (ESI): m/z=358.1 [M+1]⁺. RT=1.82 min

Step-4 The synthesis of 175-D: To a solution of 175-C (357 mg, 1.0 mmol) and 4-phenylpicolinic acid (260 mg, 1.3 mmol) in DMF (20 mL) was added HATU (570 mg, 1.5 mmol) and DIEA (1 mL), and the reaction mixture was allowed to stir at room temperature overnight. After consumption of the starting material, the reaction mixture was diluted by water (50 mL) and extracted with EtOAc (2×50 mL). The organic extract was washed by water (4×100 mL) and brine (50 mL) successively, dried over anhydrous Na₂SO₄, filtered and evaporated under reduced pressure to give a residue. The residue was purified by column chromatography on silica gel (eluting with 60% EA in PE) to give 175-D (420 mg, 78% yield) as a light-yellow solid. LC/MS (ESI): m/z=539.0 [M+1]⁺. RT=2.34 min

Step-5 The synthesis of Compound 124: To a solution of 175-D (420 mg, 0.78 mmol) in DCM (15 mL) was added TFA (5 mL), and the reaction mixture was stirred at room temperature for 2 h. After consumption of the starting material, the volatiles were evaporated under reduced pressure to give a residue. The resulted residue was dissolved in DCM (50 mL) and basified by aqueous K₂CO₃ (30 mL). The organic layer was separated, washed by water (30 mL) and brine (30 mL) successively, dried over anhydrous Na₂SO₄, filtered, evaporated under reduced pressure and purified by Prep-HPLC to give Compound 124 (210 mg, 61% yield) as a light-yellow solid.

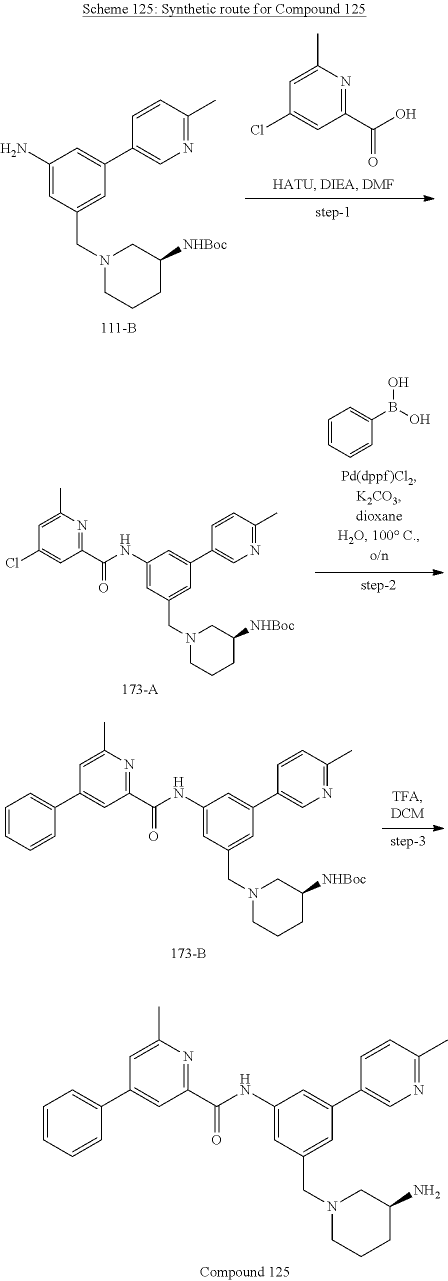 [SMILES: Cc1ccc(-c2cc(N)cc(CN3CCC[C@H](NC(=O)OC(C)(C)C)C3)c2)cn1 ; Cc1cc(Cl)cc(C(=O)O)n1 ; OB(O)c1ccccc1 ; Cc1ccc(-c2cc(CN3CCC[C@H](NC(=O)OC(C)(C)C)C3)cc(NC(=O)c3cc(Cl)cc(C)n3)c2)cn1 ; Cc1ccc(-c2cc(CN3CCC[C@H](NC(=O)OC(C)(C)C)C3)cc(NC(=O)c3cc(-c4ccccc4)cc(C)n3)c2)cn1 ; Cc1ccc(-c2cc(CN3CCC[C@H](N)C3)cc(NC(=O)c3cc(-c4ccccc4)cc(C)n3)c2)cn1]

Step-1 The synthesis of 173-A: To a solution of 111-B (396 mg, 1.0 mmol) and 4-chloro-6-methylpicolinic acid (206 g, 1.2 mmol) in DMF (40 mL) was added HATU (570 mg, 1.5 mmol) and DIEA (387 mg, 3.0 mmol), and the reaction mixture was allowed to stir at room temperature overnight. After consumption of the starting material, the reaction mixture was diluted by water (100 mL) and extracted with EtOAc (2×50 mL). The organic extract was washed by water (4×100 mL) and brine (100 mL) successively, dried over anhydrous Na₂SO₄, filtered and evaporated under reduced pressure to give a residue. The residue was purified by column chromatography on silica gel (eluting with 3% MeOH in DCM) to give 173-A (500 mg, 91% yield) as grey solid. LC/MS (ESI): m/z=550.0 [M+1]⁺. RT=2.180 min

Step-2 The synthesis of 173-B: To a mixture of 173-A (600 mg, 1.1 mmol), phenylboronic acid (201 mg, 1.65 mmol), and K₂CO₃ (455 mg, 3.3 mmol) in 1, 4-dioxane (40 mL) and H₂O (8 mL) was added Pd(dppf)Cl₂ (161 mg, 0.22 mmol), and the reaction mixture was allowed to heat at reflux and stir overnight under argon atmosphere. After cooling to room temperature, it was diluted by water (100 mL) and extracted with EtOAc (2×50 mL). The organic extract was washed by brine (100 mL), dried over anhydrous Na₂SO₄ and evaporated under reduced pressure to give a residue. The residue was purified by column chromatography on silica gel (eluting with 3% MeOH in DCM) to give 173-B (450 mg, 69% yield) as a yellow solid. LC/MS (ESI): m/z=592.1 [M+1]⁺. RT=2.273 min

Step-3 The synthesis of Compound 125: To a solution of 173-B (450 mg, 0.76 mmol) in DCM (15 mL) was added TFA (5 mL), and the reaction mixture was stirred at room temperature for 2 h. After consumption of the starting material, the volatiles were evaporated under reduced pressure to give a residue. The resulted residue was dissolved in DCM (50 mL) and basified by aqueous K₂CO₃ (20 mL). The organic layer was separated, washed by water (50 mL) and brine (50 mL) successively, dried over anhydrous Na₂SO₄, filtered and evaporated under reduced pressure, purified by pre-HPLC to give Compound 125 (144 mg, 38% yield) as a white solid.

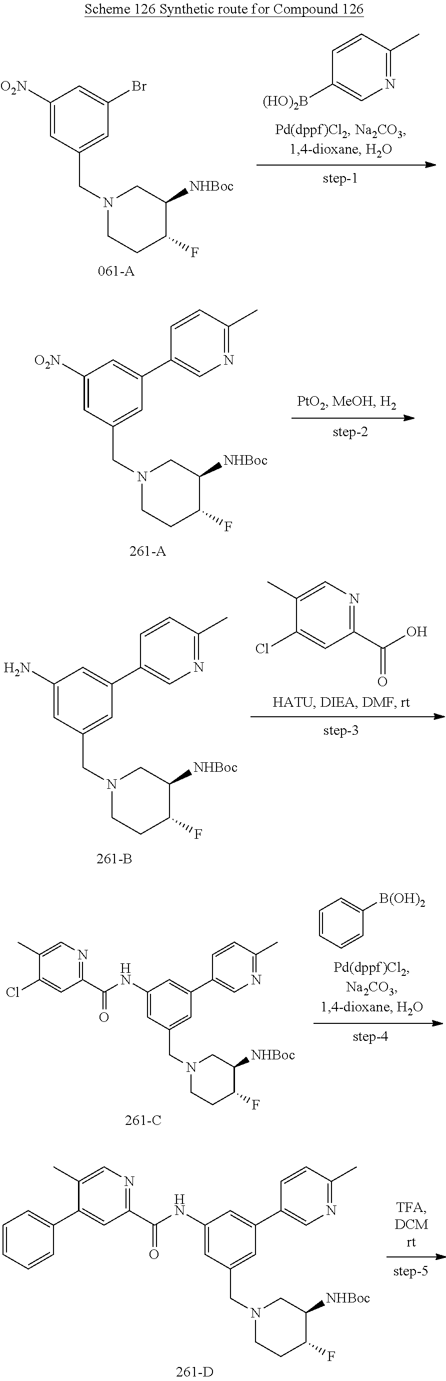

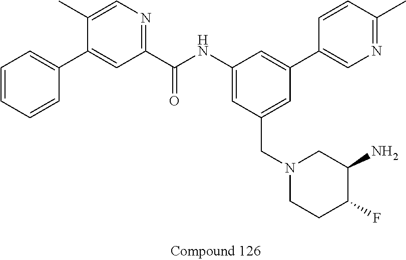 [SMILES: Cc1ccc(-c2cc(CN3CC[C@@H](F)[C@H](N)C3)cc(NC(=O)c3cc(-c4ccccc4)c(C)cn3)c2)cn1]

Step-1 The synthesis of 261-A: The title compound 260-A (370 mg, 0.83 mmol, 72% yield) was prepared according to the General Method F1 with 061-A (500 mg, 1.16 mmol), (2-methylpyridin-5-yl)boronic acid (175 mg, 1.27 mmol), Na₂CO₃ (481 mg, 3.48 mmol) and Pd(dppf)Cl₂ (180 mg, 0.12 mmol) in 1,4-dioxane (30 mL) and H₂O (6 mL). LC/MS (ESI): m/z=445.2 [M+1]⁺. RT=1.78 min

Step-2 The synthesis of 261-B: The title compound 261-B (280 mg, 0.68 mmol, 81% yield) was prepared according to the General Method D1 with 216-A (370 mg, 0.83 mmol), PtO₂ (37 mg) in MeOH (50 mL). LC/MS (ESI): m/z=415.1 [M+1]⁺. RT=1.92 min

Step-3 The synthesis of 261-C: The title compound 261-C (210 mg, 0.37 mmol, 54% yield) was prepared according to the General Method E with 261-B (280 mg, 0.68 mmol), 4-chloro-5-methylpicolinic acid (131 mg, 0.76 mmol), HATU (394 mg, 1.04 mmol) and DIEA (267 mg, 2.08 mmol) in DMF (15 mL). LC/MS (ESI): m/z=568.2 [M+1]⁺. RT=1.975 min

Step-4 The synthesis of 261-D: The title compound 261-D (110 mg, 0.18 mmol, 49% yield) was prepared according to the General Method F1 with 261-C (210 mg, 0.37 mol), phenylboronic acid (54 mg, 0.44 mmol), Na₂CO₃ (118 mg, 1.11 mmol) and Pd(dppf)Cl₂ (54 mg, 0.074 mmol) in 1,4-dioxane (30 mL) and H₂O (6 mL). LC/MS (ESI): m/z=610.2 [M+1]⁺. RT=2.063 min

Step-5 The synthesis of Compound 126: The title compound Compound 126 (74 mg, 80% yield) was prepared according to the General Method G with 261-D (110 mg, 0.18 mmol) and TFA (3 mL) in DCM (10 mL).

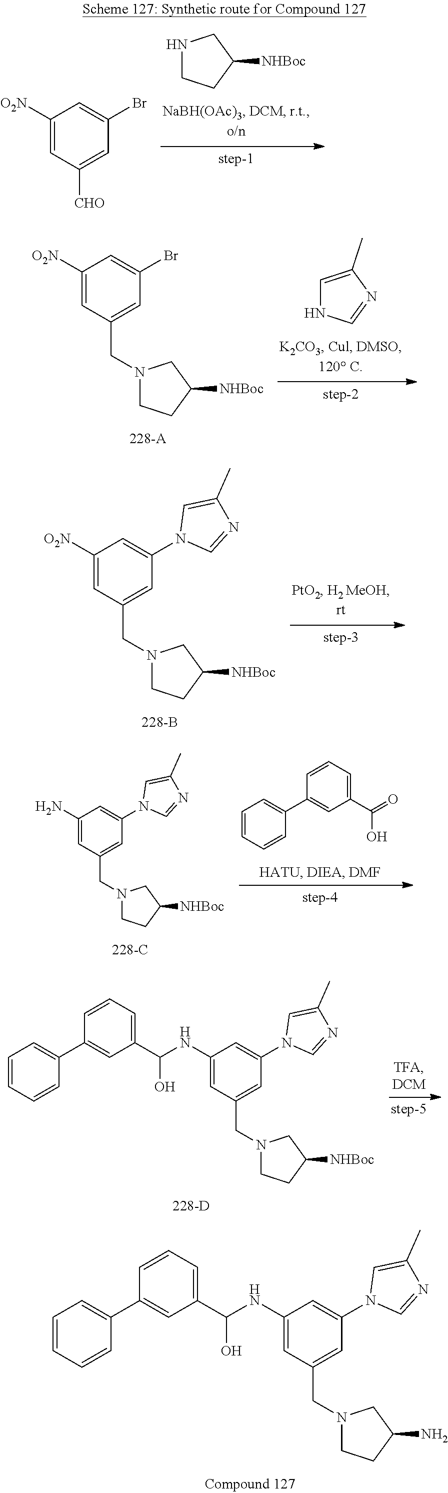

Step-1 The synthesis of 228-A: To a mixture of 3-bromo-5-nitrobenzaldehyde (1.0 g, 4.4 mmol) and tert-butyl (3S)-pyrrolidin-3-ylcarbamate (820 mg, 4.4 mmol) in DCM (50 mL) was added NaBH(OAc)₃ (4.66 g, 22 mmol) and the reaction mixture was stirred at room temperature overnight. The solvent was evaporated in vacuo to give a residue and the residue was partitioned between saturated aqueous NaHCO₃ solution (100 mL) and EA (100 mL). The mixture was stirred for 5 minutes and layers were separated. The organic phase was concentrated in vacuo to give a residue. The residue was purified by column chromatography on silica gel (eluting with 50% EA in PE) to give 228-A (1.0 g, 57% yield) as a yellow oil. LC/MS (ESI): m/z=400.0 [M+1]⁺. RT=2.295 min

Step-2 The synthesis of 228-B: A suspension of 228-A (1.0 g, 4.0 mmol), 4-methyl-1H-imidazole (1.64 g, 20 mmol), K₂CO₃ (1.10 g, 8.0 mmol), CuI (228 mg, 1.20 mmol) and quinolin-8-ol (174 mg, 1.20 mmol) in DMSO (20 mL) was heated at 120° C. and stirred overnight under nitrogen atmosphere. After cooling to room temperature, water (100 mL) was added and the mixture was extracted with EtOAc (50 mL×2). The combined organic layers were washed with water (30 mL×4) and brine (30 mL) successively, dried over Na₂SO₄ and concentrated under reduced pressure to give a residue. The residue was purified by column chromatography on silica gel (eluting with 3% MeOH in DCM) to give 228-B (700 mg, 87% yield) as clear oil. LC/MS (ESI): m/z=402.1 [M+1]⁺. RT=1.78 min

Step-3 The synthesis of 228-C: To a solution of 228-B (700 mg, 1.74 mmol) in MeOH (50 mL) was added PtO₂ (100 mg), and the reaction mixture was stirred at room temperature for 3 h under hydrogen atmosphere. After consumption of the starting material, the reaction mixture was filtered through a celite pad to remove the solid and the filtrate was evaporated under reduced pressure to give crude 228-C (600 mg, 93% yield) as gray solid. LC/MS (ESI): m/z=372.1 [M+1]⁺. RT=1.58 min

Step-4 The synthesis of 228-D: To a solution of 228-C (300 mg, 0.80 mmol) and [1,1′-biphenyl]-3-carboxylic acid (208 mg, 1.04 mmol) in DMF (20 mL) was added HATU (570 mg, 1.04 mmol) and DIEA (270 mg, 2.08 mmol), and the reaction mixture was allowed to stir at room temperature overnight. After consumption of the starting material, the reaction mixture was diluted by water (100 mL) and extracted with EtOAc (50 mL×2). The organic extract was washed by water (50 mL×4) and brine (50 mL) successively, dried over anhydrous Na₂SO₄, filtered and evaporated under reduced pressure to give a residue. The residue was purified by column chromatography on silica gel (eluting with 50% EA in PE) to give 228-D (200 mg, 45% yield) as a white solid. LC/MS (ESI): m/z=552.0 [M+1]⁺. RT=2.21 min

Step-5 The synthesis of Compound 127:

To a solution of 228-D (200 mg, 0.36 mmol) in DCM (15 mL) was added TFA (3 mL), and the reaction mixture was stirred at room temperature for 2 h. After consumption of the starting material, the volatiles were evaporated under reduced pressure to give a residue. The resulted residue was dissolved in DCM (30 mL) and basified by aqueous K₂CO₃ (30 mL). The organic layer was separated, washed by water (30 mL) and brine (30 mL) successively, dried over anhydrous Na₂SO₄, filtered, evaporated under reduced pressure and purified by Prep-HPLC to give Compound 127 (15 mg, 9.2% yield) as a white solid.

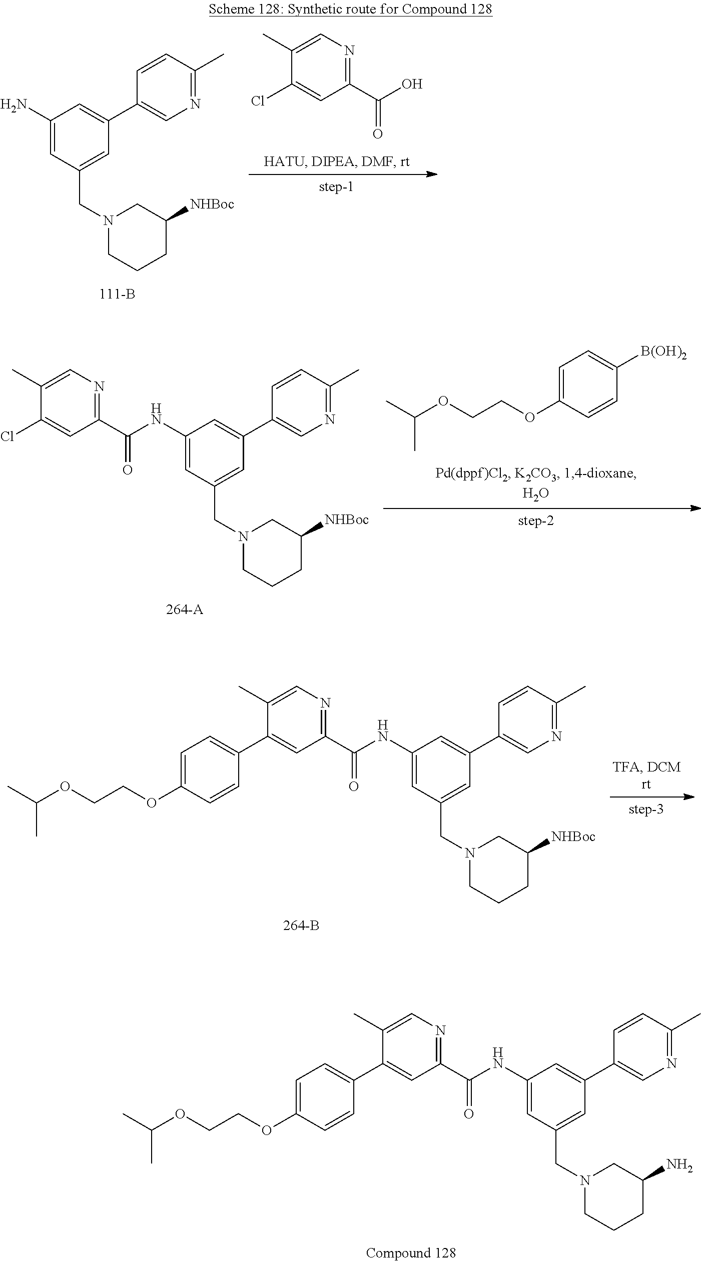

Step-1 The synthesis of 264-A: The title compound 264-A (292 mg, 0.53 mmol, 53% yield) was prepared according to the General Method E with 111-B (400 mg, 1.0 mmol), 4-chloro-5-methylpicolinic acid (191 mg, 1.1 mmol), HATU (576 mg, 1.5 mmol) and DIEA (391 mg, 3.0 mmol) in DMF (15 mL). LC/MS (ESI): m/z=550.1 [M+1]⁺. RT=2.174 min

Step-2 The synthesis of 264-B: The title compound 264-B (40 mg, 0.06 mmol, 11% yield) was prepared according to the General Method F1 with 264-A (292 mg, 0.53 mmol), 4-(2-isopropoxyethoxy)phenylboronic acid (142 mg, 0.64 mmol), Na₂CO₃ (169 mg, 1.59 mmol) and Pd(dppf)Cl₂ (39 mg, 0.053 mmol) in 1,4-dioxane (30 mL) and H₂O (6 mL). LC/MS (ESI): m/z=694.2 [M+1]⁺. RT=2.164 min

Step-3 The synthesis of Compound 128: The title compound Compound 128 (7.5 mg, 21.12% yield) was prepared according to the General Method G with 264-B (40 mg, 0.06 mmol) and TFA (3 mL) in DCM (10 mL).

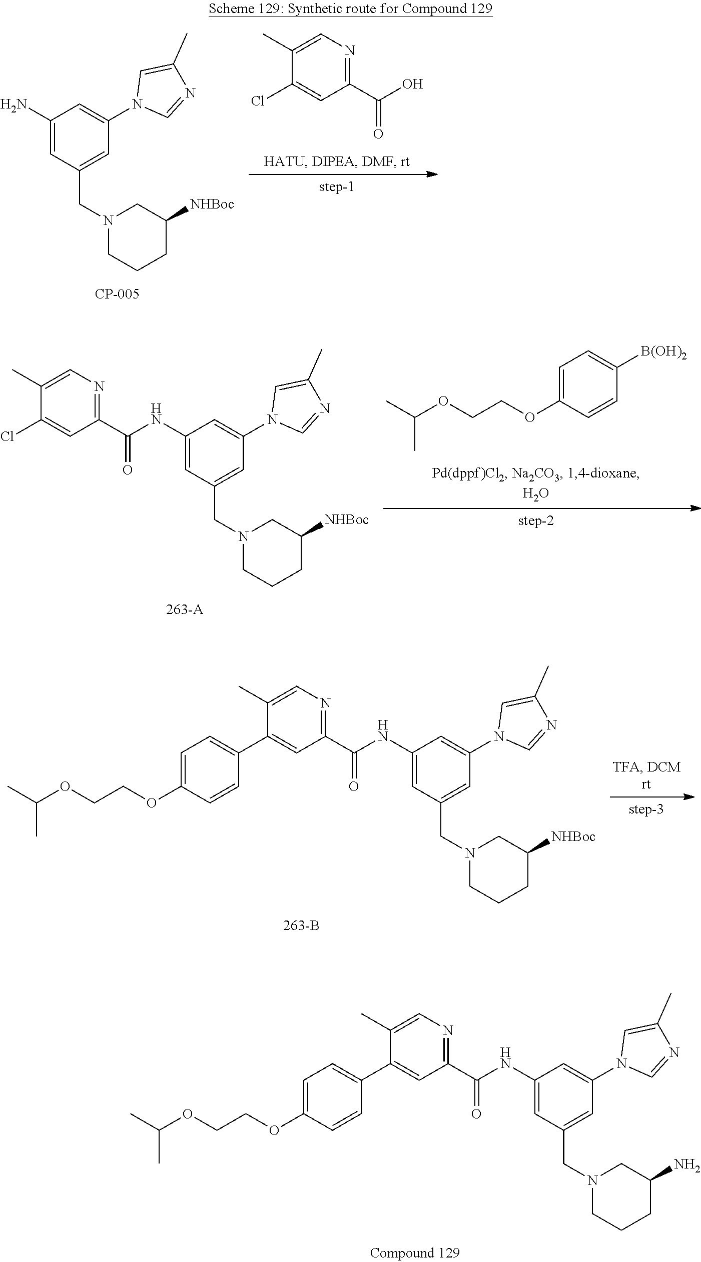 [SMILES: Cc1cn(-c2cc(N)cc(CN3CCC[C@H](NC(=O)OC(C)(C)C)C3)c2)cn1 ; Cc1cnc(C(=O)O)cc1Cl ; Cc1cn(-c2cc(CN3CCC[C@H](NC(=O)OC(C)(C)C)C3)cc(NC(=O)c3cc(Cl)c(C)cn3)c2)cn1 ; CC(C)OCCOc1ccc(B(O)O)cc1 ; Cc1cn(-c2cc(CN3CCC[C@H](NC(=O)OC(C)(C)C)C3)cc(NC(=O)c3cc(-c4ccc(OCCOC(C)C)cc4)c(C)cn3)c2)cn1 ; Cc1cn(-c2cc(CN3CCC[C@H](N)C3)cc(NC(=O)c3cc(-c4ccc(OCCOC(C)C)cc4)c(C)cn3)c2)cn1]

Step-1 The synthesis of 263-A: The title compound 263-A (190 mg, 0.35 mmol, 34% yield) was prepared according to the General Method E with CP-005 (400 mg, 1.04 mmol), 4-chloro-5-methylpicolinic acid (197 mg, 1.14 mmol), HATU (593 mg, 1.56 mmol) and DIEA (402 mg, 3.12 mmol) in DMF (15 mL). LC/MS (ESI): m/z=539.0 [M+1]⁺. RT=2.31 min

Step-2 The synthesis of 263-B: The title compound 263-B (90 mg, 0.13 mmol, 37.5% yield) was prepared according to the General Method F1 with 263-A (190 mg, 0.35 mmol), 4-(2-isopropoxyethoxy)phenylboronic acid (94 mg, 0.42 mmol), Na₂CO₃ (111 mg, 1.05 mmol) and Pd(dppf)Cl₂ (51 mg, 0.07 mmol) in 1,4-dioxane (30 mL) and H₂O (6 mL). LC/MS (ESI): m/z=683.0 [M+1]⁺. RT=2.20 min

Step-3 The synthesis of Compound 129: The title compound Compound 129 (11 mg, 14.5% yield) was prepared according to the General Method G with 263-B (90 mg, 0.13 mmol) and TFA (3 mL) in DCM (10 mL).

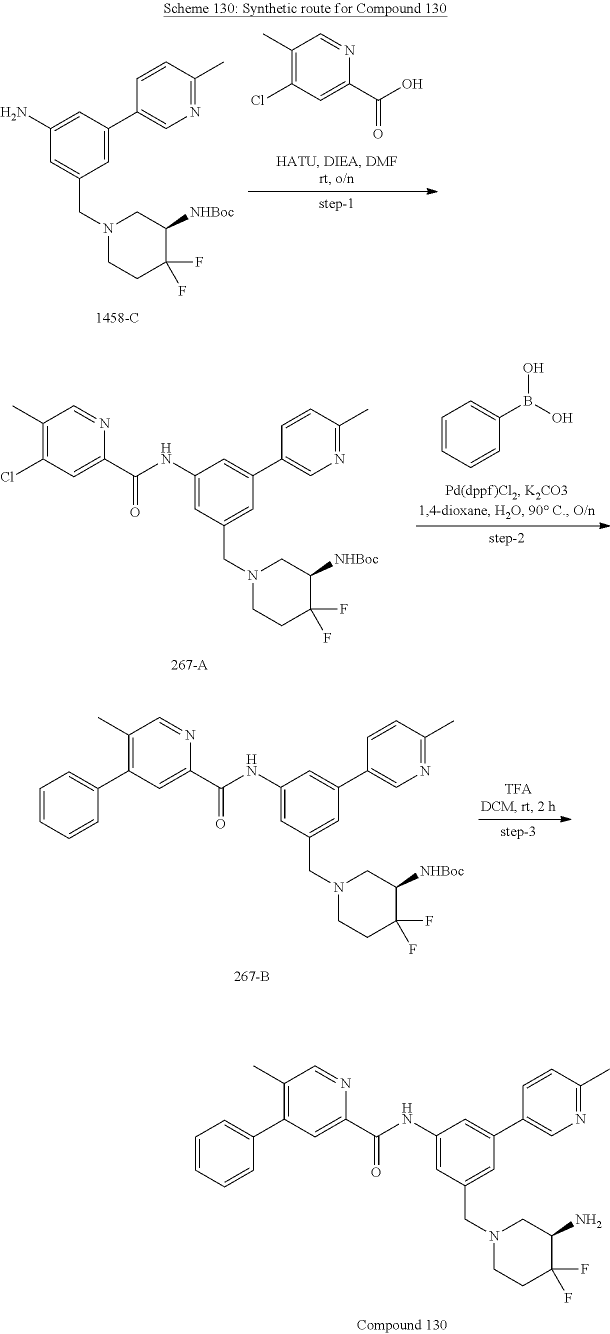

Step-1 The synthesis of 267-A: To a solution of 1458-C (600 mg, 1.39 mmol) and 4-chloro-5-methylpicolinic acid (238 mg, 1.39 mmol) in DMF (40 mL) was added HATU (802 mg, 2.08 mmol) and DIEA (538 mg, 4.17 mmol), and the reaction mixture was allowed to stir at room temperature overnight. After consumption of the starting material, the reaction mixture was diluted by water (100 mL) and extracted with EtOAc (2×50 mL). The organic extract was washed by water (4×100 mL) and brine (100 mL) successively, dried over anhydrous Na₂SO₄, filtered and evaporated under reduced pressure to give a residue. The residue was purified by column chromatography on silica gel (eluting with 3% MeOH in DCM) to give 267-A (765 mg, 94% yield) as grey solid. LC/MS (ESI): m/z=586.0 [M+1]⁺. RT=2.160 min

Step-2 The synthesis of 267-B: To a mixture of 267-A (378 mg, 0.65 mmol), phenylboronic acid (118 mg, 0.97 mmol), and K₂CO₃ (267 mg, 1.94 mmol) in 1, 4-dioxane (30 mL) and H₂O (6 mL) was added Pd(dppf)Cl₂ (94 mg, 0.13 mmol), and the reaction mixture was allowed to heat at reflux and stir overnight under argon atmosphere. After cooling to room temperature, it was diluted by water (100 mL) and extracted with EtOAc (2×50 mL). The organic extract was washed by brine (100 mL), dried over anhydrous Na₂SO₄ and evaporated under reduced pressure to give a residue. The residue was purified by column chromatography on silica gel (eluting with 3% MeOH in DCM) to give 267-B (250 mg, 62% yield) as a yellow solid. LC/MS (ESI): m/z=628.1 [M+1]⁺. RT=2.219 min

Step-3 The synthesis of Compound 130:

To a solution of 267-B (450 mg, 0.76 mmol) in DCM (10 mL) was added TFA (3 mL), and the reaction mixture was stirred at room temperature for 2 h. After consumption of the starting material, the volatiles were evaporated under reduced pressure to give a residue. The resulted residue was dissolved in DCM (50 mL) and basified by aqueous K₂CO₃ (20 mL). The organic layer was separated, washed by water (50 mL) and brine (50 mL) successively, dried over anhydrous Na₂SO₄, filtered and evaporated under reduced pressure, purified by reversed phase pre-HPLC to give Compound 130 (117 mg, 55% yield) as a white solid.

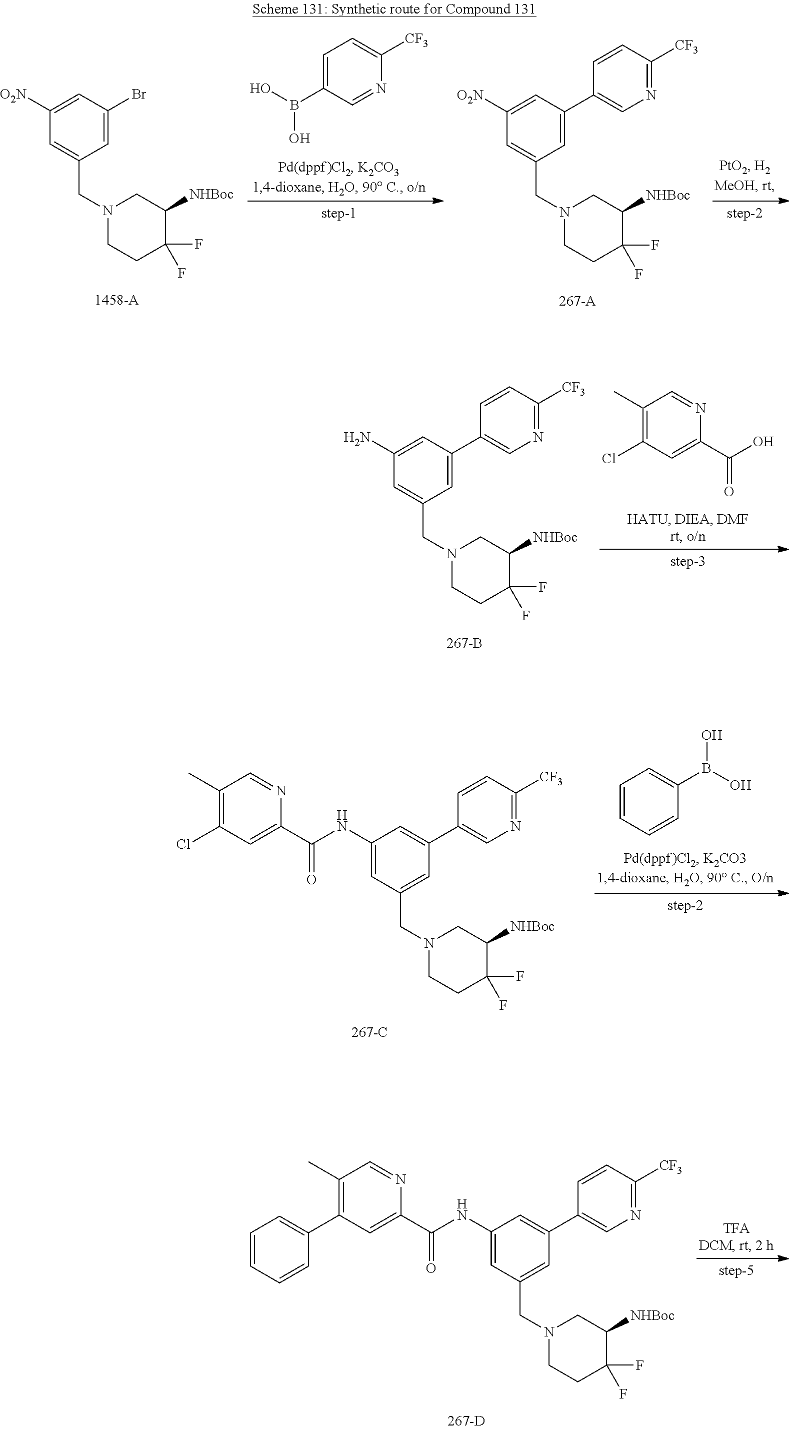

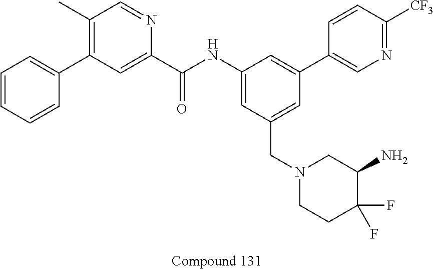 [SMILES: Cc1cnc(C(=O)Nc2cc(CN3CCC(F)(F)[C@H](N)C3)cc(-c3ccc(C(F)(F)F)nc3)c2)cc1-c1ccccc1]

Step-1 The synthesis of 287-A: To a mixture of 1458-A (480 mg, 1.06 mmol), 2-(trifluoromethyl)pyridin-5-ylboronic acid (224 mg, 1.2 mmol), and K₂CO₃ (438 mg, 3.18 mmol) in 1, 4-dioxane (30 mL) and H₂O (6 mL) was added Pd(dppf)Cl₂ (78 mg, 0.11 mmol), and the reaction mixture was allowed to heat at reflux and stir overnight under nitrogen atmosphere. After cooling to room temperature, it was diluted by water (100 mL) and extracted with EtOAc (2×50 mL). The organic extract was washed by brine (100 mL), dried over anhydrous Na₂SO₄ and evaporated under reduced pressure to give a residue. The residue was purified by column chromatography on silica gel (eluting with 3% MeOH in DCM) to give 287-A (470 mg, 86% yield) as a white solid. LC/MS (ESI): m/z=517.0 [M+1]⁺. RT=2.148 min

Step-2 The synthesis of 287-B: To a solution of 287-A (470 mg, 0.91 mmol) in MeOH (40 mL) was added PtO₂ (47 mg), and the reaction mixture was stirred at room temperature for 2 hours under hydrogen atmosphere. After consumption of the starting material, the reaction mixture was filtered through a celite pad to remove the solid and the filtrate was evaporated under reduced pressure to give crude 287-B (550 mg, 62% yield) as grey solid. LC/MS (ESI): m/z=487.3 [M+1]⁺. RT=2.242 min

Step-3 The synthesis of 287-C: To a solution of 287-B (275 mg, 0.57 mmol) and 4-chloro-5-methylpicolinic acid (98 mg, 0.57 mmol) in DMF (30 mL) was added HATU (323 mg, 0.85 mmol) and DIEA (220 mg, 1.70 mmol), and the reaction mixture was allowed to stir at room temperature overnight. After consumption of the starting material, the reaction mixture was diluted by water (100 mL) and extracted with EtOAc (2×50 mL). The organic extract was washed by water (4×100 mL) and brine (100 mL) successively, dried over anhydrous Na₂SO₄, filtered and evaporated under reduced pressure to give a residue. The residue was purified by column chromatography on silica gel (eluting with 3% MeOH in DCM) to give 287-C (340 mg, 93% yield) as a white solid. LC/MS (ESI): m/z=639.9 [M+1]⁺. RT=2.271 min

Step-4 The synthesis of 287-D: To a mixture of 287-C (400 mg, 0.63 mmol), phenylboronic acid (115 mg, 0.94 mmol), and K₂CO₃ (260 mg, 1.88 mmol) in 1, 4-dioxane (30 mL) and H₂O (6 mL) was added Pd(dppf)Cl₂ (92 mg, 0.13 mmol), and the reaction mixture was allowed to heat at reflux and stir overnight under argon atmosphere. After cooling to room temperature, it was diluted by water (100 mL) and extracted with EtOAc (2×50 mL). The organic extract was washed by brine (100 mL), dried over anhydrous Na₂SO₄ and evaporated under reduced pressure to give a residue. The residue was purified by column chromatography on silica gel (eluting with 3% MeOH in DCM) to give 287-D (180 mg, 42% yield) as grey solid. LC/MS (ESI): m/z=682.0 [M+1]⁺. RT=2.334 min

Step-5 The synthesis of Compound 131: To a solution of 287-D (180 mg, 0.26 mmol) in DCM (10 mL) was added TFA (3 mL), and the reaction mixture was stirred at room temperature for 2 h. After consumption of the starting material, the volatiles were evaporated under reduced pressure to give a residue. The resulted residue was dissolved in DCM (50 mL) and basified by aqueous K₂CO₃ (10 mL). The organic layer was separated, washed by water (50 mL) and brine (50 mL) successively, dried over anhydrous Na₂SO₄, filtered and evaporated under reduced pressure, and purified by reversed phase pre-HPLC to give Compound 131 (78 mg, 52% yield) as a white solid.

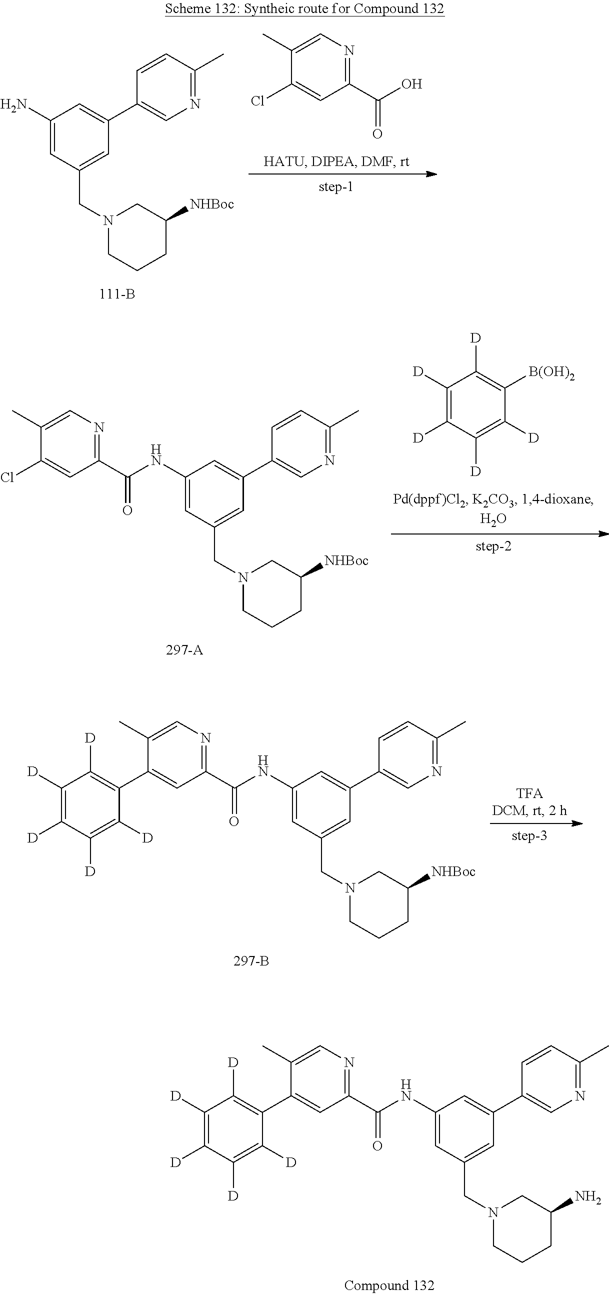

Step-1 The synthesis of 297-A: The title compound 297-A (700 mg, 1.17 mmol, 48% yield) was prepared according to the General Method E with 111-B (1.0 g, 2.42 mmol), 4-chloro-5-methylpicolinic acid (537 mg, 2.66 mmol), HATU (1.38 g, 3.63 mmol) and DIEA (467 mg, 3.63 mmol) in DMF (15 mL). LC/MS (ESI): m/z=550.0 [M+1]⁺. RT=2.164 min

Step-2 The synthesis of 297-B: The title compound 297-B (320 mg, 0.53 mmol, 45% yield) was prepared according to the General Method F1 with 297-A (700 mg, 1.17 mmol), (pentadeuterophenyl)boronic acid (223 mg, 1.76 mmol), K₂CO₃ (344 mg, 3.51 mmol) and Pd(dppf)Cl₂ (86 mg, 0.117 mmol) in 1,4-dioxane (30 mL) and H₂O (6 mL). LC/MS (ESI): m/z=597.2 [M+1]⁺. RT=2.25 min

Step-3 The synthesis of Compound 132: The title compound Compound 132 (100 mg, 38% yield) was prepared according to the General Method G with 297-B (320 mg, 0.53 mmol) and TFA (3 mL) in DCM (10 mL).

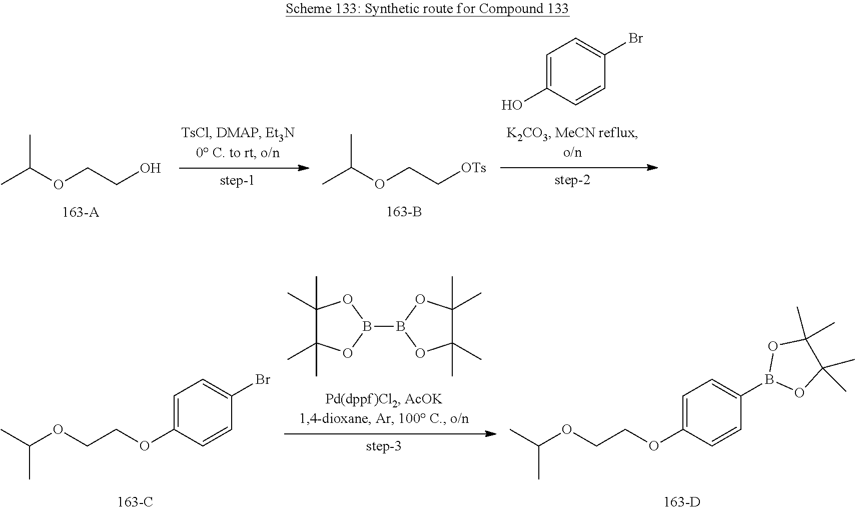

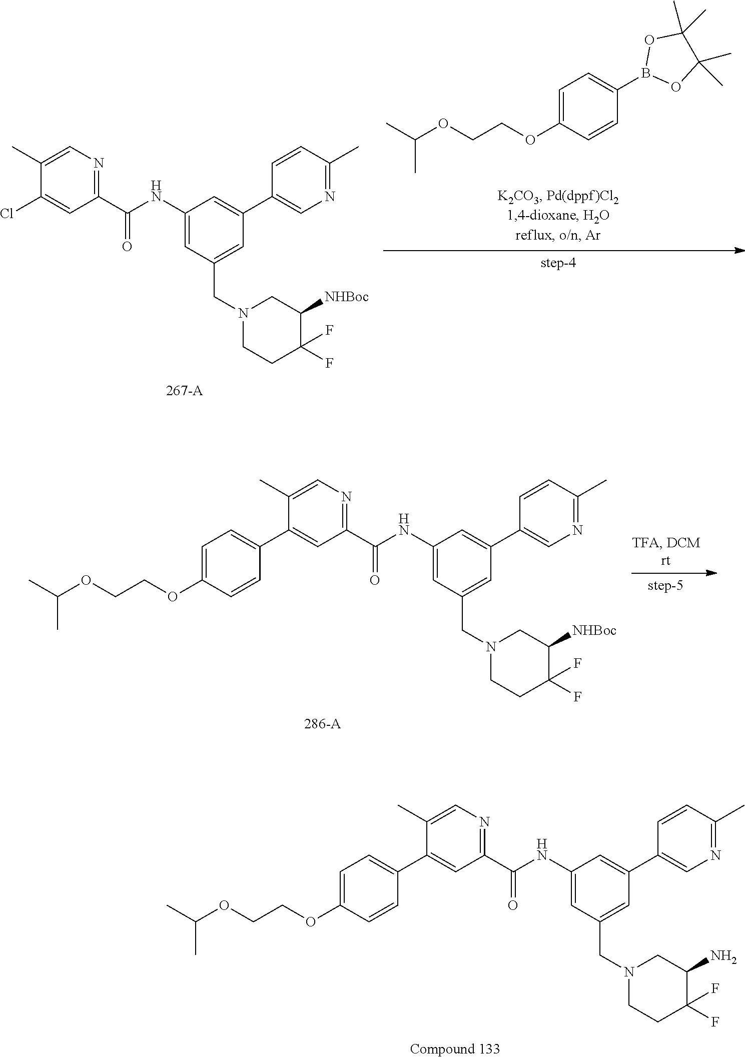

Step-1 The synthesis of 163-B: To a mixture of 2-isopropoxyethanol (4.0 g, 38.4 mmol), DMAP (8.76 g, 46.1 mmol), and Et₃N (7.76 g, 76.8 mmol) in dry DCM (200 mL) was added TsCl (8.76 g, 46.1 mmol) slowly at 0° C., and the reaction mixture was allowed to stir overnight under argon atmosphere at room temperature. After consumption of the starting materials, it was evaporated under reduced pressure to give a residue. The residue was dissolved in EA (200 mL) and washed with brine (200 mL). The organic layer was dried over anhydrous Na₂SO₄ and evaporated under reduced pressure to give 163-B (5.6 g, 56% yield) as a white solid. LC/MS (ESI): m/z=276.1 [M+18]⁺. RT=1.900 min

Step-2 The synthesis of 163-C: To a mixture of 163-B (5.6 g, 21.7 mmol), 4-bromophenol (3.94 g, 22.8 mmol) in MeCN (100 mL) was added K₂CO₃ (4.49 g, 32.6 mmol), and the reaction mixture was allowed to heat at reflux and stirred overnight under argon atmosphere at room temperature. After consumption of the starting materials, it was evaporated under reduced pressure to give a residue. The residue was dissolved in EA (200 mL) and washed with brine (200 mL). The organic layer was dried over anhydrous Na₂SO₄ and evaporated under reduced pressure to give 163-C (5.06 g, 90% yield) as a white solid.

Step-3 The synthesis of 163-D: To a mixture of 163-C (5.06 g, 19.6 mmol), bis(pinacolato)diborane (7.47 g, 29.4 mmol), and AcOK (3.85 g, 39.2 mmol) in 1, 4-dioxane (120 mL) was added Pd(dppf)Cl₂ (2.8 g, 3.9 mmol), and the reaction mixture was allowed to heat at 85° C. and stir overnight under argon atmosphere. After cooling to room temperature, it was diluted by water (200 mL) and extracted with EtOAc (2×200 mL). The organic extract was washed by brine (200 mL), dried over anhydrous Na₂SO₄ and evaporated under reduced pressure to give a residue. The residue was purified by column chromatography on silica gel (eluting with 10% EA in PE) to give 163-D (4.8 g, 80% yield) as gray solid. LC/MS (ESI): m/z=324.1 [M+18]⁺. RT=2.166 min

Step-4 The synthesis of 286-A: To a mixture of 267-A (200 mg, 0.34 mmol), 163-D (125 mg, 0.41 mmol), and K₂CO₃ (142 mg, 1.02 mmol) in 1, 4-dioxane (30 mL) and H₂O (6 mL) was added Pd(dppf)Cl₂ (50 mg, 0.068 mmol), and the reaction mixture was allowed to heat at reflux and stir overnight under argon atmosphere. After cooling to room temperature, it was diluted by water (100 mL) and extracted with EtOAc (2×50 mL). The organic extract was washed by brine (100 mL), dried over anhydrous Na₂SO₄ and evaporated under reduced pressure to give a residue. The residue was purified by column chromatography on silica gel (eluting with 3% MeOH in DCM) to give 286-A (120 mg, 48% yield) as gray solid. LC/MS (ESI): m/z=730.0 [M+1]⁺. RT=2.604 min

Step-5 The synthesis of Compound 133: To a solution of 286-A (120 mg, 0.16 mmol) in DCM (10 mL) was added TFA (3 mL), and the reaction mixture was stirred at room temperature for 2 h. After consumption of the starting material, the volatiles were evaporated under reduced pressure to give a residue. The resulted residue was dissolved in DCM (30 mL) and basified by aqueous K₂CO₃ (20 mL). The organic layer was separated, washed by water (30 mL) and brine (30 mL) successively, dried over anhydrous Na₂SO₄, filtered and evaporated under reduced pressure, and purified by reversed phase pre-HPLC to give Compound 133 (46 mg, 45% yield) as a gray solid.

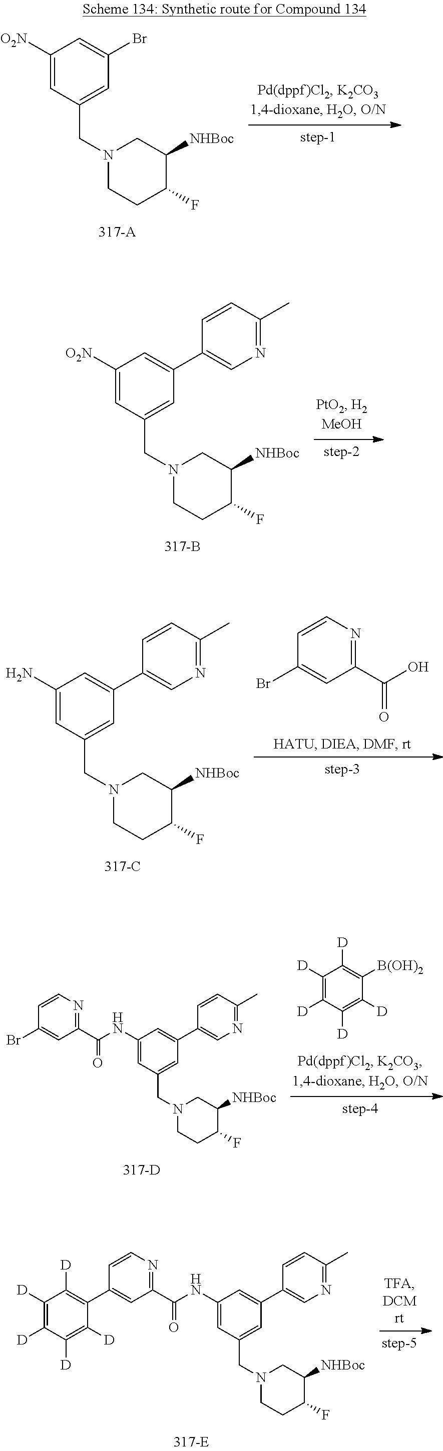

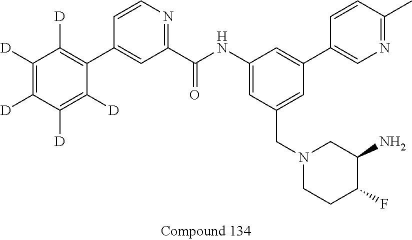 [SMILES: [2H]c1c([2H])c([2H])c(-c2ccnc(C(=O)Nc3cc(CN4CC[C@@H](F)[C@H](N)C4)cc(-c4ccc(C)nc4)c3)c2)c([2H])c1[2H]]

Step-1 The synthesis of 317-B: The title compound 317-B (1.15 g, 2.60 mmol, 68% yield) was prepared according to the General Method F1 with 317-A (1.65 g, 3.8 mmol), (2-methylpyridin-5-yl)boronic acid (628 mg, 4.56 mmol), Na₂CO₃ (1.13 g, 11.4 mmol) and Pd(dppf)Cl₂ (278 mg, 0.38 mmol) in 1,4-dioxane (30 mL) and H₂O (6 mL). LC/MS (ESI): m/z=445.2 [M+1]⁺. RT=1.80 min

Step-2 The synthesis of 317-C: The title compound 317-C (1.0 g, 2.42 mmol, 93% yield) was prepared according to the General Method D1 with 317-B (1.15 g, 2.60 mmol), PtO₂ (120 mg) in MeOH (80 mL). LC/MS (ESI): m/z=415.1 [M+1]⁺. RT=1.75 min

Step-3 The synthesis of 317-D: The title compound 317-D (700 mg, 1.17 mmol, 48% yield) was prepared according to the General Method E with 317-C (1.0 g, 2.42 mmol), 4-bromopicolinic acid (537 mg, 2.66 mmol), HATU (1.38 g, 3.63 mmol) and DIEA (467 mg, 3.63 mmol) in DMF (15 mL). LC/MS (ESI): m/z=598.2 [M+1]⁺. RT=1.93 min

Step-4 The synthesis of 317-E: The title compound 317-E (320 mg, 0.53 mmol, 45% yield) was prepared according to the General Method F1 with 317-D (700 mg, 1.17 mmol), phenyl-d5-boronic acid (223 mg, 1.76 mmol), K₂CO₃ (484 mg, 3.51 mmol) and Pd(dppf)Cl₂ (86 mg, 0.117 mmol) in 1,4-dioxane (30 mL) and H₂O (6 mL). LC/MS (ESI): m/z=601.0 [M+1]⁺. RT=2.42 min

Step-5 The synthesis of Compound 134: The title compound Compound 134 (100 mg, 38% yield) was prepared according to the General Method G with 317-E (320 mg, 0.53 mmol) and TFA (3 mL) in DCM (10 mL).

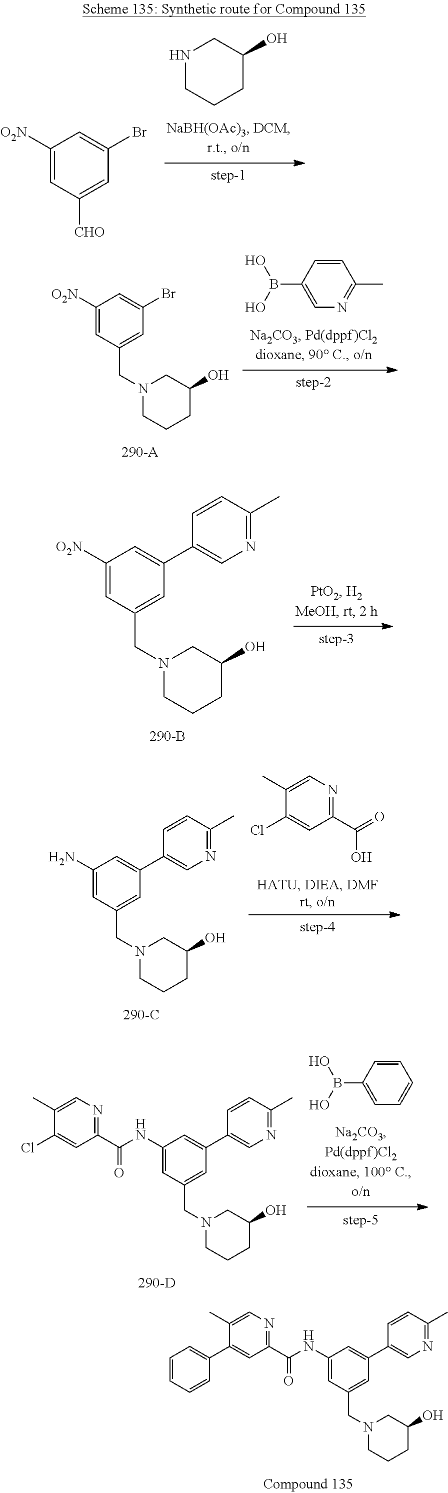

Step-1 The synthesis of 290-A: To a mixture of 3-bromo-5-nitrobenzaldehyde (800 mg, 3.5 mmol) and (3S)-piperidin-3-ol (390 mg, 3.5 mmol) in DCM (50 mL) was added NaBH(OAc)₃ (3.0 g, 14.0 mmol) and the reaction mixture was stirred at room temperature overnight. The solvent was evaporated in vacuo to a residue and the residue was partitioned between saturated aqueous NaHCO₃ solution (100 mL) and EA (100 mL). The mixture was stirred for 5 minutes and the layers were separated. The organic phase was concentrated in vacuo to give a residue. The residue was purified by column chromatography on silica gel (eluting with 50% EA in PE) to give 290-A (400 mg, 36% yield) as a white solid. LC/MS (ESI): m/z=315.0 [M+1]⁺. RT=1.789 min

Step-2 The synthesis of 290-B: A suspension of 290-A (400 mg, 1.27 mmol), (2-methylpyridin-5-yl)boronic acid (260 mg, 1.91 mmol), Na₂CO₃ (270 mg, 2.54 mmol) and Pd (dppf)Cl₂ (100 mg, 0.13 mmol) in dioxane (50 mL) and water (10 mL) was heated at 90° C. and stirred overnight under nitrogen atmosphere. After cooling to room temperature, water (100 mL) was added and the mixture was extracted with EtOAc (50 mL×2). The combined organic layers were washed with water (30 mL) and brine (30 mL) successively, dried over Na₂SO₄ and concentrated under reduced pressure to give a residue. The residue was purified by column chromatography on silica gel (eluting with EA) to give 290-B (300 mg, 72% yield) as a white solid. LC/MS (ESI): m/z=328.1 [M+1]⁺. RT=1.655 min

Step-3 The synthesis of 290-C: To a solution of 290-B (300 mg, 0.92 mmol) in MeOH (50 mL) was added PtO₂ (30 mg), and the reaction mixture was stirred at room temperature for 2 h under hydrogen atmosphere. After consumption of the starting material, the reaction mixture was filtered through a celite pad to remove the solid and the filtrate was evaporated under reduced pressure to give crude 290-C (200 mg, 73% yield) as gray solid. LC/MS (ESI): m/z=298.2 [M+1]⁺. RT=1.39 min

Step-4 The synthesis of 290-D: To a solution of 290-C (200 mg, 0.67 mmol) and 4-chloro-5-methylpicolinic acid (138 mg, 0.81 mmol) in DMF (20 mL) was added HATU (510 mg, 1.34 mmol) and DIEA (166 mg, 1.34 mmol), and the reaction mixture was allowed to stir at room temperature overnight. After consumption of the starting material, the reaction mixture was diluted by water (100 mL) and extracted with EtOAc (50 mL×2). The organic extract was washed by water (100 mL×4) and brine (50 mL) successively, dried over anhydrous Na₂SO₄, filtered and evaporated under reduced pressure to give a residue. The residue was purified by Pre-HPLC to give 290-D (150 mg, 50% yield) as a white solid. LC/MS (ESI): m/z=451.1 [M+1]⁺. RT=1.85 min

Step-5 The synthesis of Compound 135: A suspension of 290-D (150 mg, 0.33 mmol), phenylboronic acid (80 mg, 0.66 mmol), Na₂CO₃ (70 mg, 0.66 mmol) and Pd (dppf)Cl₂ (74 mg, 0.1 mmol) in dioxane (30 mL) and water (5 mL) was heated at 100° C. and stirred overnight under nitrogen atmosphere. After cooling to room temperature, water (100 mL) was added and the mixture was extracted with EtOAc (50 mL×2). The combined organic layers were washed with water (30 mL) and brine (30 mL) successively, dried over Na₂SO₄ and concentrated under reduced pressure to give a residue. The residue was purified by Pre-HPLC to give Compound 135 (18 mg, 11% yield) as a white solid.

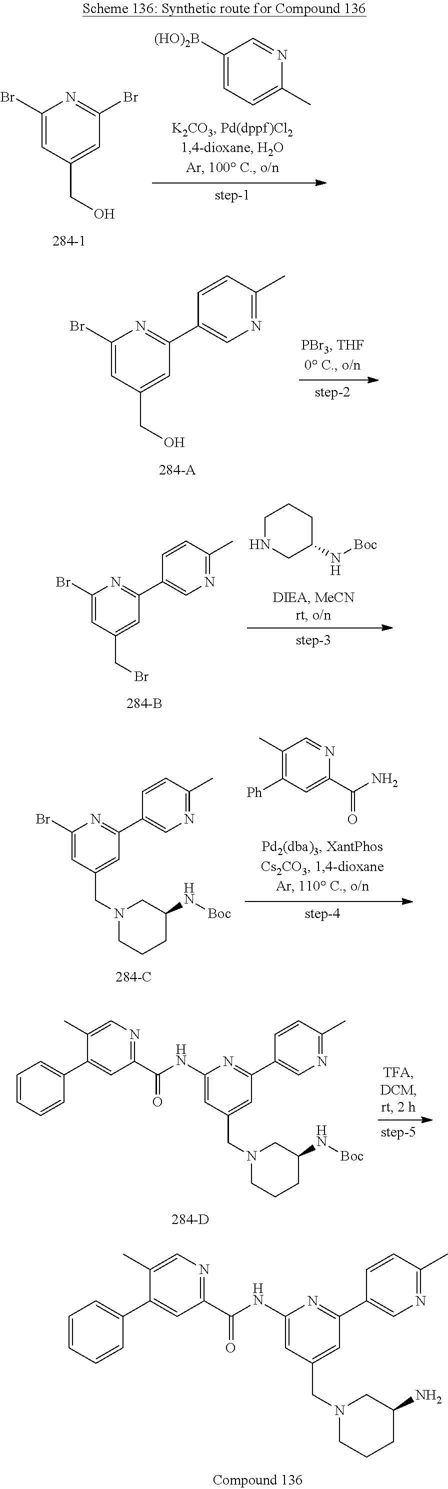

Step-1 The synthesis of 284-A: A mixture of 284-1 (2.93 g, 10.97 mmol), (2-methylpyridin-5-yl)boronic acid (1.5 g, 10.97 mmol), K₂CO₃ (4.55 g, 32.9 mmol) and Pd(dppf)Cl₂ (803 mg, 1.1 mmol) in 1, 4-dioxane (100 mL) and H₂O (20 mL) was heated to 100° C. and stirred overnight under argon atmosphere. After cooling to room temperature, water (50 mL) was added and the mixture was extracted with EtOAc (50 mL×2). The combined organic layers were washed with brine (50 mL), dried over Na₂SO₄ and concentrated. The residue was purified by silica gel column (eluting with 88% EA in PE) to give 284-A (552 mg, 18% yield) as a white solid. LC/MS (ESI): m/z=279.0 [M+1]⁺. RT=1.637 min

Step-2 The synthesis of 284-B: To a solution of 284-A (552 mg, 1.98 mmol) in THF (40 mL) was added PBr₃ (0.2 mL, 1.97 mmol) at 0° C., and the reaction mixture was stirred at room temperature overnight. After consumption of the starting material, the volatiles were evaporated under reduced pressure to give a residue. The resulted residue was dissolved in EtOAc (30 mL) and basified by aqueous Na₂CO₃ (30 mL). The organic layer was separated, washed by water (30 mL) and brine (30 mL) successively, dried over anhydrous Na₂SO₄, filtered, evaporated under reduced pressure to give 284-B (600 mg, 90% yield) as a yellow liquid. LC/MS (ESI): m/z=342.9 [M+1]⁺. RT=1.915 min

Step-3 The synthesis of 284-C: To a solution of 284-B (676 mg, 1.98 mmol) and tert-butyl (3S)-piperidin-3-ylcarbamate (395 mg, 1.98 mmol) in MeCN (40 mL) was added DIEA (1.2 mL, 6.92 mmol), and the reaction mixture was stirred at room temperature overnight. After consumption of the starting material, the volatiles were evaporated under reduced pressure to give a residue. The resulted residue was added with water (50 mL), extracted with EtOAc (30 mL×2). The combined organic layers were washed with brine (50 mL), dried over Na₂SO₄ and concentrated. The residue was purified by silica gel column chromatography (eluting with 80% EA in PE) to give 284-C (499 mg, 55% yield) as a white solid. LC/MS (ESI): m/z=461.0 [M+1]⁺. RT=2.066 min

Step-4 The synthesis of 284-D: A mixture of 284-C (200 mg, 0.43 mmol), 5-methyl-4-phenylpicolinamide (101 mg, 0.48 mmol), Pd₂(dba)₃ (40 mg, 0.04 mmol), XantPhos (51 mg, 0.09 mmol) and Cs₂CO₃ (422 mg, 1.3 mmol) in 1, 4-dioxane (20 mL) was heated at 110° C. and stirred overnight under argon. After cooling to room temperature, water (50 mL) was added and the mixture was extracted with EtOAc (50 mL×2). The combined organic layers were washed with brine (50 mL), dried over Na₂SO₄ and concentrated. The residue was purified by silica gel column chromatography (eluting with 60% EA in PE) to give 284-D (193 mg, 75% yield) as a yellow solid. LC/MS (ESI): m/z=593.0 [M+1]⁺. RT=2.702 min

Step-5 The synthesis of Compound 136: To a solution of 284-D (193 mg, 0.33 mmol) in DCM (30 mL) was added TFA (4 mL), and the reaction mixture was stirred at room temperature for 2 h. After consumption of the starting material, the volatiles were evaporated under reduced pressure to give a residue. The resulted residue was dissolved in DCM (30 mL) and basified by aqueous K₂CO₃ (30 mL). The organic layer was separated, washed by brine (30 mL), dried over anhydrous Na₂SO₄, filtered, evaporated under reduced pressure and purified by Prep-HPLC to give Compound 136 (50 mg, 31% yield) as a white solid.

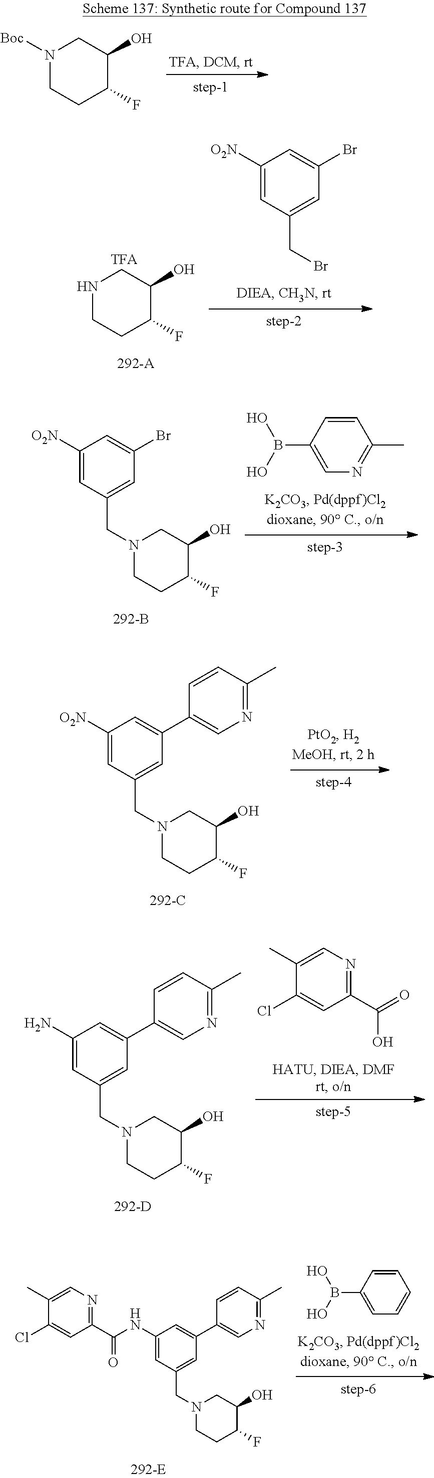

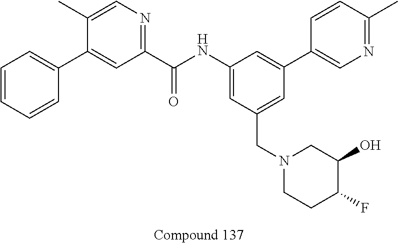 [SMILES: Cc1ccc(-c2cc(CN3CC[C@@H](F)[C@H](O)C3)cc(NC(=O)c3cc(-c4ccccc4)c(C)cn3)c2)cn1]

Step-1 The synthesis of 292-A: To a mixture of tert-butyl (3R,4R)-4-fluoro-3-hydroxypiperidine-1-carboxylate (1.0 g, 4.56 mmol) in DCM (30 mL) was added TFA (10 mL). The reaction was stirred at room temperature for 2 h. The mixture was concentrated in vacuo to give the crude product 292-A (980 mg, 99%) as a yellow liquid. LC/MS (ESI): m/z=120.3 [M+1]⁺. RT=0.26 min

Step-2 The synthesis of 292-B: To a mixture of 292-A (543 mg, 4.56 mmol) and 1-bromo-3-(bromomethyl)-5-nitrobenzene (3.34 g, 11.31 mmol) in MeCN (30 mL) was added DIEA (3 mL). The reaction mixture was stirred at room temperature overnight. After consumption of the starting material, the mixture was concentrated in vacuo, then water (30 mL) was added and the mixture was extracted with EA (3×30 mL). The combined organic layers were washed with brine (80 mL), dried over Na₂SO₄ and concentrated in vacuo to give a residue. The residue was purified by column chromatography on silica gel (eluting with PE:EA=3:1) to give 292-B (1.10 g, 73% yield) as a yellow liquid. LC/MS (ESI): m/z=333.0 [M+1]⁺. RT=1.800 min

Step-3 The synthesis of 292-C: To a mixture of 292-B (300 mg, 0.9 mmol) and 2-methylpyridin-5-ylboronic acid (185 mg, 1.4 mmol) in 1,4-dioxane/H₂O (20 mL/4 mL) was added Pd(dppf)Cl₂ (30 mg) and K₂CO₃ (248 mg, 1.8 mmol). The reaction mixture was stirred at 90° C. overnight. After consumption of the starting material, water (30 mL) was added and the mixture was extracted with EA (3×50 mL). The combined organic layers were washed with brine (80 mL), dried over Na₂SO₄ and concentrated in vacuo to give a residue. The residue was purified by column chromatography on silica gel (eluting with PE:EA=1:9) to give 292-C (220 mg, 63% yield) as a yellow liquid. LC/MS (ESI): m/z=346.1 [M+1]⁺. RT=1.651 min

Step-4 The synthesis of 292-D: To a solution of 292-C (220 mg, 0.64 mmol) in MeOH (20 mL) was added PtO₂ (20 mg). The reaction mixture was stirred at room temperature for 5 h under H₂. After consumption of the starting material, the mixture was filtered and concentrated in vacuo to give crude 292-D (210 mg) as a yellow liquid. LC/MS (ESI): m/z=316.2 [M+1]⁺. RT=1.209 min

Step-5 The synthesis of 292-E: To a solution of 292-D (210 mg, 0.67 mmol) and 4-chloro-5-methylpicolinic acid (137 mg, 0.80 mmol) in DMF (20 mL) was added HATU (380 mg, 1.0 mmol) and DIEA (0.5 mL). The reaction mixture was stirred at room temperature overnight. After consumption of the starting material, water (40 mL) was added and the mixture was extracted with EA (3×50 mL). The combined organic layers were washed with brine (100 mL), dried over Na₂SO₄ and concentrated in vacuo to give a residue. The residue was purified by column chromatography on silica gel (eluting with PE:EA=1:3) to give 292-E (200 mg, 52% yield) as a yellow liquid. LC/MS (ESI): m/z=469.2 [M+1]⁺. RT=1.659 min

Step-6 The synthesis of Compound 137: To a mixture of 292-E (200 mg, 0.43 mmol) and phenylboronic acid (68 mg, 0.56 mmol) in 1,4-dioxane/H₂O (20 mL/4 mL) was added Pd(dppf)Cl₂ (63 mg, 0.086 mmol) and K₂CO₃ (119 mg, 0.86 mmol). The reaction mixture was stirred at 90° C. overnight. After consumption of the starting material, water (50 mL) was added and the mixture was extracted with EA (3×50 mL). The combined organic layers were washed with brine (100 mL), dried over Na₂SO₄ and concentrated in vacuo to give a residue. The residue was purified by Prep-HPLC to give Compound 137 (42 mg, 26% yield) as a white solid.

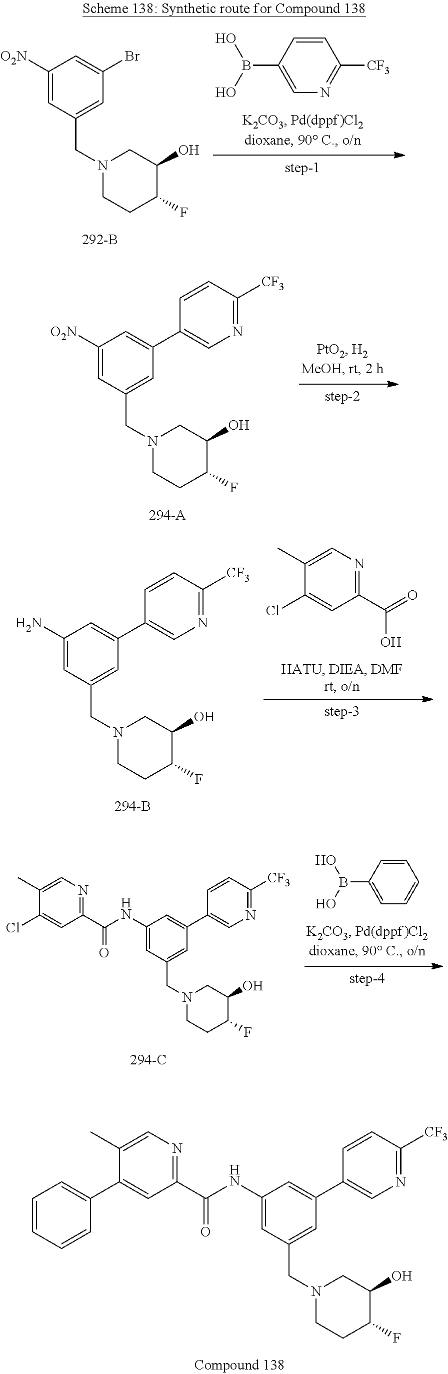

Step-1 The synthesis of 294-A: To a mixture of 292-B (300 mg, 0.9 mmol) and 2-(trifluoromethyl)pyridin-5-ylboronic acid (267 mg, 1.4 mmol) in 1,4-dioxane/H₂O (20 mL/4 mL) was added Pd(dppf)Cl₂ (30 mg) and K₂CO₃ (248 mg, 1.8 mmol). The reaction mixture was stirred at 90° C. overnight. After consumption of the starting material, water (50 mL) was added and the mixture was extracted with EA (3×50 mL). The combined organic layers were washed with brine (100 mL), dried over Na₂SO₄ and concentrated in vacuo to give a residue. The residue was purified by column chromatography on silica gel (eluting with PE:EA=2:1) to give 294-A (330 mg, 92% yield) as a yellow liquid. LC/MS (ESI): m/z=400.0 [M+1]⁺. RT=1.853 min

Step-2 The synthesis of 294-B: To a mixture of 294-A (330 mg, 0.83 mmol) in MeOH (20 mL) was added PtO₂ (33 mg). The reaction mixture was stirred at room temperature for 5 h under H₂. After consumption of the starting material, the mixture was filtered and concentrated in vacuo to give crude 294-B (275 mg, 99%) as a yellow solid. LC/MS (ESI): m/z=370.2 [M+1]⁺. RT=1.442 min

Step-3 The synthesis of 294-C: To a solution of 294-B (275 mg, 0.83 mmol) and 4-chloro-5-methylpicolinic acid (181 mg, 1.06 mmol) in DMF (20 mL) was added HATU (463 mg, 1.22 mmol) and DIEA (0.5 mL). The reaction mixture was stirred at room temperature overnight. After consumption of the starting material, water 50 mL) was added and the mixture was extracted with EA (3×50 mL). The combined organic layers were washed with water (4×100 mL) and brine (100 mL) successively, dried over Na₂SO₄ and concentrated in vacuo to give a residue. The residue was purified by column chromatography on silica gel (eluting with PE:EA=1:1) to give 294-C (230 mg, 51% yield) as a white solid. LC/MS (ESI): m/z=523.1 [M+1]⁺. RT=1.878 min

Step-4 The synthesis of Compound 138: To a mixture of 294-C (230 mg, 0.44 mmol) and phenylboronic acid (70 mg, 0.57 mmol) in 1,4-dioxane/H₂O (20 mL/4 mL) was added Pd(dppf)Cl₂ (65 mg, 0.088 mmol) and K₂CO₃ (121 mg, 0.88 mmol). The reaction mixture was stirred at 90° C. overnight. After consumption of the starting material, water (50 mL) was added and the mixture was extracted with EA (2×50 mL). The combined organic layers were washed with brine (50 mL), dried over Na₂SO₄ and concentrated in vacuo to give a residue. The residue was purified by Prep-HPLC to give Compound 138 (107 mg, 44% yield) as a white solid.

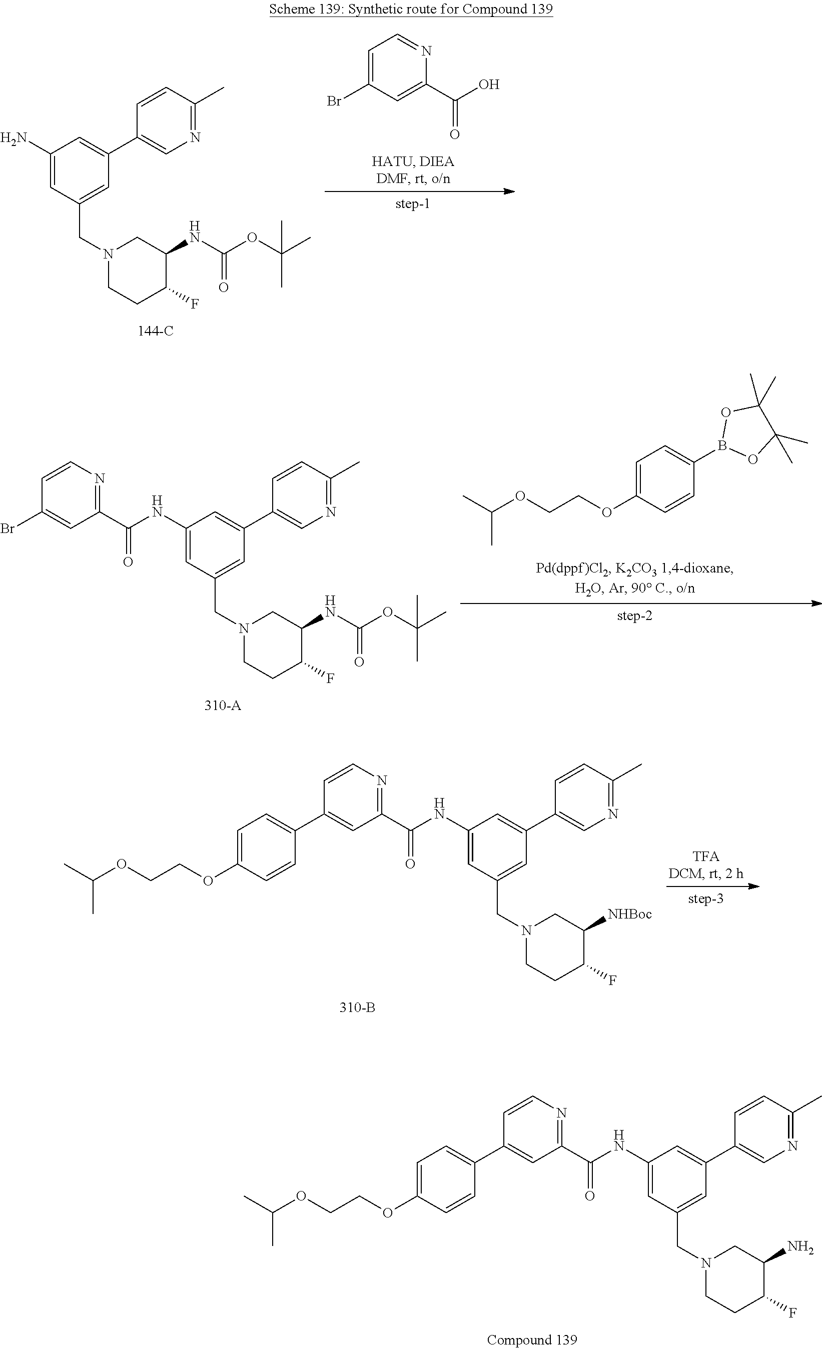

Step-1 The synthesis of 310-A: To a solution of 144-C (250 mg, 0.6 mmol) and 4-bromopicolinic acid (146 mg, 0.72 mmol) in DMF (30 mL) was added HATU (342 mg, 0.9 mmol) and DIEA (232 mg, 1.8 mmol), and the reaction mixture was allowed to stir at room temperature overnight. After consumption of the starting material, the reaction mixture was diluted by water (100 mL) and extracted with EtOAc (2×50 mL). The organic extract was washed by water (4×100 mL) and brine (100 mL) successively, dried over anhydrous Na₂SO₄, filtered and evaporated under reduced pressure to give a residue. The residue was purified by column chromatography on silica gel (eluting with 3% MeOH in DCM) to give 310-A (300 mg, 83% yield) as a yellow solid. LC/MS (ESI): m/z=598.0 [M+1]⁺. RT=2.090 min

Step-2 The synthesis of 310-B: To a mixture of 310-A (300 mg, 0.5 mmol), 2-(4-(2-isopropoxyethoxy)phenyl)-4,4,5,5-tetramethyl-1,3,2-dioxaborolane (184 mg, 0.6 mmol), and K₂CO₃ (207 mg, 1.5 mmol) in 1, 4-dioxane (30 mL) and H₂O (6 mL) was added Pd(dppf)Cl₂ (37 mg, 0.05 mmol), and the reaction mixture was allowed to heat at reflux and stir overnight under argon atmosphere. After cooling to room temperature, it was diluted by water (100 mL) and extracted with EtOAc (2×50 mL). The organic extract was washed by brine (100 mL), dried over anhydrous Na₂SO₄ and evaporated under reduced pressure to give a residue. The residue was purified by column chromatography on silica gel (eluting with 3% MeOH in DCM) to give 310-B (260 mg, 75% yield) as a white solid. LC/MS (ESI): m/z=698.0 [M+1]⁺. RT=2.514 min

Step-3 The synthesis of Compound 139: To a solution of 310-B (260 mg, 0.37 mmol) in DCM (10 mL) was added TFA (3 mL), and the reaction mixture was stirred at room temperature for 2 h. After consumption of the starting material, the volatiles were evaporated under reduced pressure to give a residue. The resulted residue was dissolved in DCM (50 mL) and basified by aqueous K₂CO₃ (30 mL). The organic layer was separated, washed by water (50 mL) and brine (50 mL) successively, dried over anhydrous Na₂SO₄, filtered and evaporated under reduced pressure, and purified by reversed phase pre-HPLC to give Compound 139 (94.4 mg, 43% yield) as a white solid.

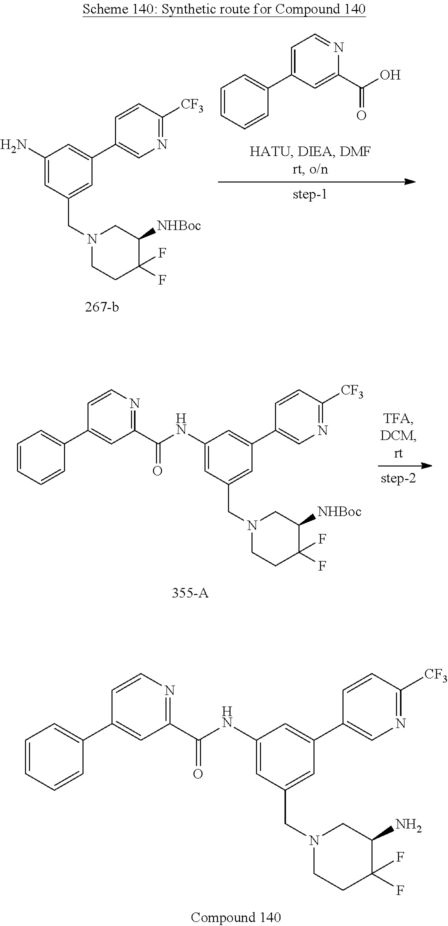

Step-1 The synthesis of 355-A: To a mixture of 287-B (200 mg, 0.41 mmol), 4-phenylpicolinic acid (98 mg, 0.49 mmol) in DMF (30 mL) was added HATU (224 mg, 0.62 mmol) and DIEA (159 mg, 1.23 mmol), and the reaction mixture was allowed to stir at room temperature overnight. After consumption of the starting material, the reaction mixture was diluted by water (100 mL) and extracted with EtOAc (2×50 mL). The organic extract was washed by water (4×100 mL) and brine (100 mL) successively, dried over anhydrous Na₂SO₄, filtered and evaporated under reduced pressure to give a residue. The residue was purified by column chromatography on silica gel (eluting with 3% MeOH in DCM) to give 355-A (200 mg, 73% yield) as a white solid. LC/MS (ESI): m/z=667.9 [M+1]⁺. RT=2.279 min

Step-2 The synthesis of Compound 140: To a solution of 355-A (200 mg, 0.30 mmol) in DCM (10 mL) was added TFA (3 mL), and the reaction mixture was stirred at room temperature for 2 h. After consumption of the starting material, the volatiles were evaporated under reduced pressure to give a residue. The resulted residue was dissolved in DCM (50 mL) and basified by aqueous K₂CO₃ (30 mL). The organic layer was separated, washed by water (50 mL) and brine (50 mL) successively, dried over anhydrous Na₂SO₄, filtered and evaporated under reduced pressure, and purified by reversed phase pre-HPLC to give Compound 140 (87 mg, 51% yield) as a white solid.

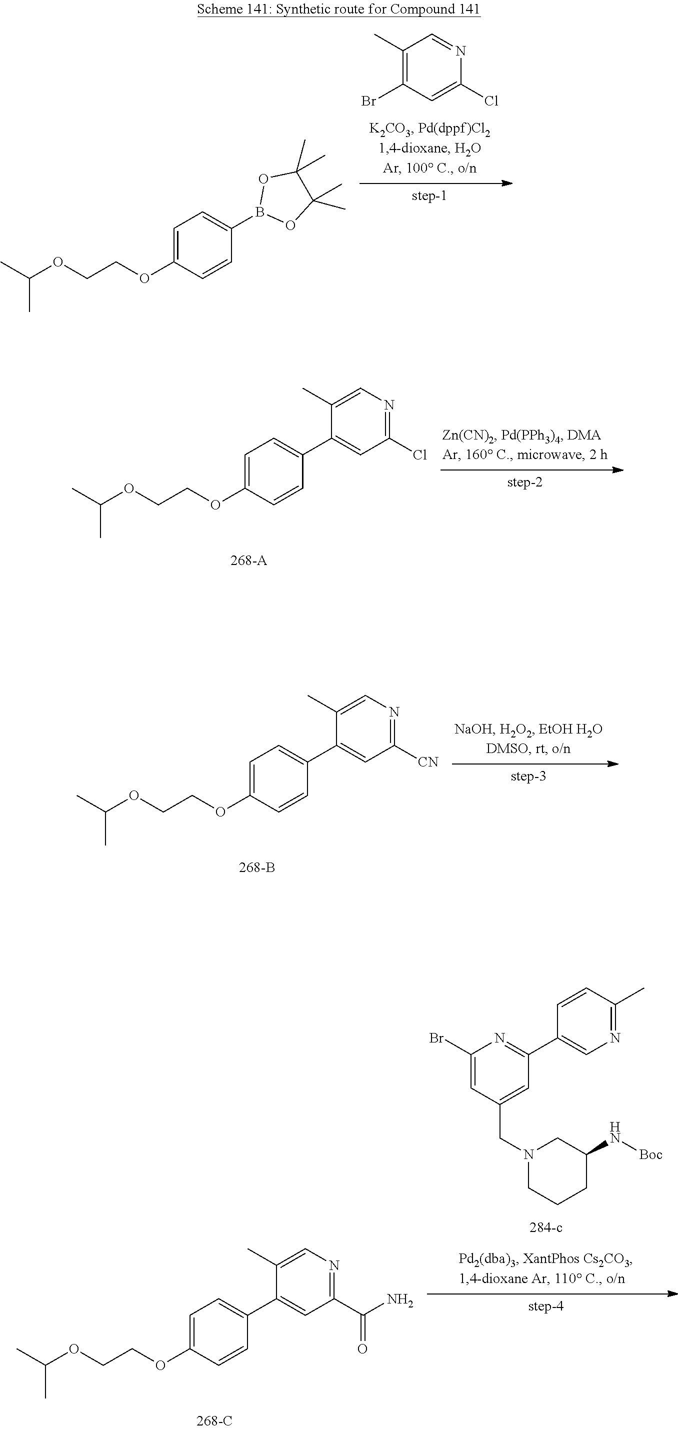

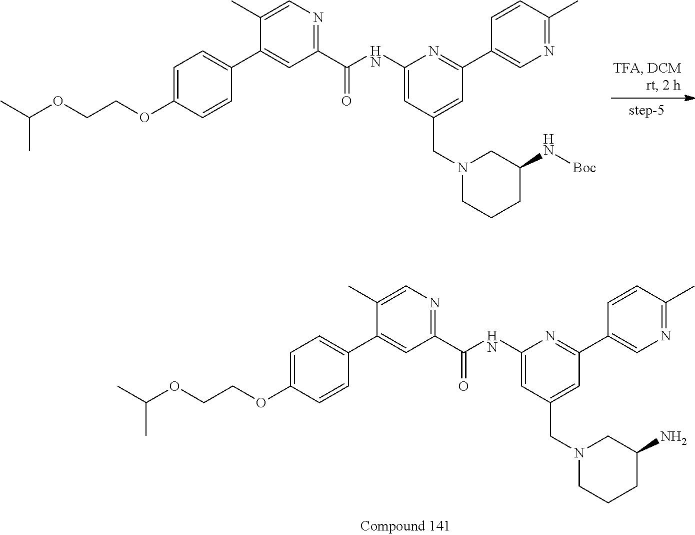 [SMILES: Cc1ccc(-c2cc(CN3CCC[C@H](NC(=O)OC(C)(C)C)C3)cc(NC(=O)c3cc(-c4ccc(OCCOC(C)C)cc4)c(C)cn3)n2)cn1 ; Cc1ccc(-c2cc(CN3CCC[C@H](N)C3)cc(NC(=O)c3cc(-c4ccc(OCCOC(C)C)cc4)c(C)cn3)n2)cn1]

Step-1 The synthesis of 268-A: To a mixture of 2-(4-(2-isopropoxyethoxy)phenyl)-4,4,5,5-tetramethyl-1,3,2-dioxaborolane (1.48 g, 4.84 mmol), 4-bromo-2-chloro-5-methylpyridine (1.0 g, 4.84 mmol), K₂CO₃ (2.0 g, 14.5 mmol) and Pd(dppf)Cl₂ (351 mg, 0.48 mmol) in 1, 4-dioxane (80 mL) and H₂O (16 mL) was heated at 100° C. and stirred overnight under argon. After cooling to room temperature, water was added and the mixture was extracted with EtOAc (50 mL×2), the combined organic layers were washed with brine (50 mL), dried over Na₂SO₄ and concentrated. The residue was purified by silica gel column chromatography (eluting with 10% EA in PE) to give 268-A (651 mg, 44% yield) as a colorless liquid. LC/MS (ESI): m/z=306.1 [M+1]⁺. RT=2.077 min

Step-2 The synthesis of 268-B: To a mixture of 268-A (300 mg, 0.98 mmol), Zn(CN)₂ (230 mg, 1.96 mmol) and Pd(PPh₃)₄ (453 mg, 0.39 mmol) in DMA (10 mL) was heated at 160° C. and stirred for 2 hours under argon with microwave. After cooling to room temperature, water was added and the mixture was extracted with EtOAc (50 mL×3), the combined organic layers were washed with brine (50 mL×4), dried over Na₂SO₄ and concentrated to give crude 268-B (291 mg, 99% yield) as a yellow gel. LC/MS (ESI): m/z=297.1 [M+1]⁺. RT=1.999 min

Step-3 The synthesis of 268-C: To a solution of 268-B (291 mg, 0.98 mmol) and NaOH (40 mg, 0.98 mmol) in EtOH (20 mL), H₂O (2 mL) and DMSO (40 mL) was added H₂O₂ (7.0 mL, 6.87 mmol), and the reaction mixture was stirred at room temperature overnight. After consumption of the starting material, the volatiles were evaporated under reduced pressure to give a residue. The resulted residue was diluted with water (30 mL), extracted with EtOAc (30 mL×3). The combined organic layers were washed with brine (50 mL), dried over Na₂SO₄ and concentrated. The residue was purified by silica gel column chromatography (eluting with 33% EA in PE) to give 268-C (241 mg, 78% yield) as a white solid. LC/MS (ESI): m/z=315.1 [M+1]⁺. RT=1.793 min

Step-4 The synthesis of 268-D: To a mixture of 268-C (98 mg, 0.31 mmol), 284-C (120 mg, 0.26 mmol), Pd₂(dba)₃ (24 mg, 0.03 mmol), XantPhos (30 mg, 0.05 mmol) and Cs₂CO₃ (254 mg, 0.78 mmol) in 1, 4-dioxane (20 mL) was heated at 110° C. and stirred overnight under argon. After cooling to room temperature, water was added and the mixture was extracted with EtOAc (50 mL×2), the combined organic layers were washed with brine (50 mL), dried over Na₂SO₄ and concentrated. The residue was purified by silica gel column chromatography (eluting with 50% EA in PE) to give 268-D (77 mg, 42% yield) as a white solid. LC/MS (ESI): m/z=695.3 [M+1]⁺. RT=2.261 min

Step-5 The synthesis of Compound 141: To a solution of 268-D (77 mg, 0.11 mmol) in DCM (20 mL) was added TFA (2 mL), and the reaction mixture was stirred at room temperature for 2 h. After consumption of the starting material, the volatiles were evaporated under reduced pressure to give a residue. The resulted residue was dissolved in DCM (30 mL) and basified by aqueous K₂CO₃ (30 mL). The organic layer was separated, washed by water (30 mL) and brine (30 mL) successively, dried over anhydrous Na₂SO₄, filtered, evaporated under reduced pressure, and purified by reversed phase Prep-HPLC to give Compound 141 (20 mg, 30% yield) as a white solid.

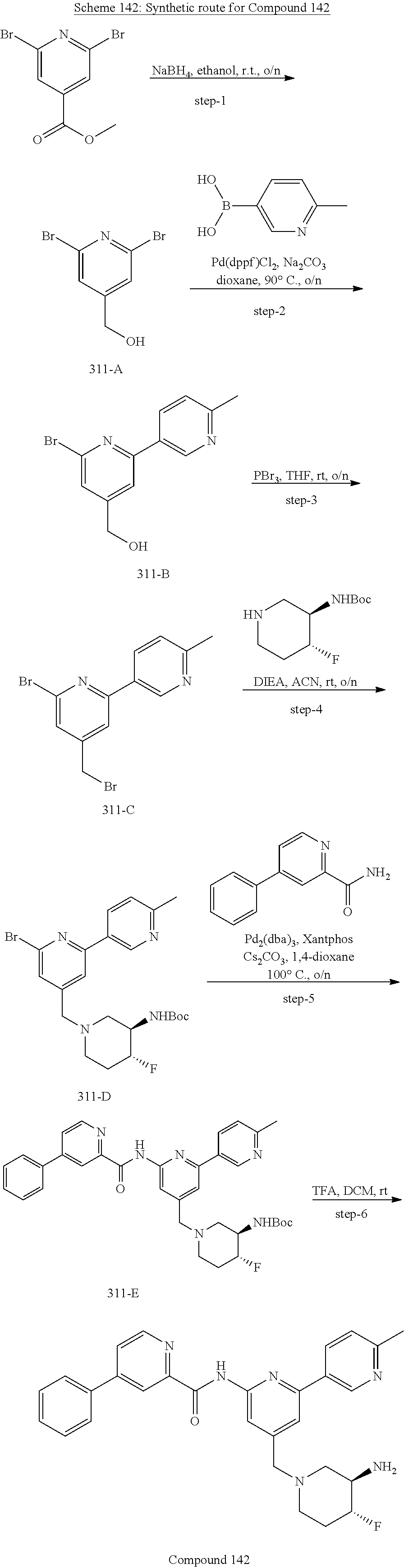

Step-1 The synthesis of 311-A: To a solution of methyl 2, 6-dibromoisonicotinate (4.0 g, 13.7 mmol) in ethanol (100 mL) was added NaBH₄ (2.08 g, 54.8 mmol) slowly and the reaction mixture was stirred at room temperature overnight. The solvent was evaporated in vacuo to give a residue and the residue was purified by column chromatography on silica gel (eluting with 20% EA in PE) to give 311-A (2.0 g, 55% yield) as a white solid. LC/MS (ESI): m/z=267.9 [M+1]⁺. RT=1.54 min

Step-2 The synthesis of 311-B: A suspension of 311-A (2.0 g, 7.5 mmol), (2-methylpyridin-5-yl)boronic acid (1.24 g, 9.0 mmol), Na₂CO₃ (1.59 g, 15.0 mmol) and Pd (dppf)Cl₂ (560 mg, 0.75 mmol) in dioxane (100 mL) and water (20 mL) was heated at 90° C. and stirred overnight under nitrogen atmosphere. After cooling to room temperature, water (100 mL) was added and the mixture was extracted with EtOAc (100 mL×2). The combined organic layers were washed with water (50 mL) and brine (50 mL) successively, dried over Na₂SO₄ and concentrated under reduced pressure to give a residue. The residue was purified by column chromatography on silica gel (eluting with 50% EA in PE) to give 311-B (600 mg, 29% yield) as a white solid. LC/MS (ESI): m/z=279.0 [M+1]⁺. RT=1.54 min

Step-3 The synthesis of 311-C: To a solution of 311-B (600 mg, 2.14 mmol) in THF (50 mL) was added PBr₃ (3.5 g, 12.84 mmol), and the reaction mixture was stirred at room temperature overnight. After consumption of the starting material, the reaction mixture was evaporated under reduced pressure to give crude 311-C as a residue. The residue was used for the next step directly. LC/MS (ESI): m/z=342.9 [M+1]⁺. RT=1.92 min

Step-4 The synthesis of 311-D: To a solution of crude 311-C in ACN (100 mL) was added DIEA (2.58 g, 20 mmol) and tert-butyl ((3R,4R)-4-fluoropiperidin-3-yl)carbamate (1.09 g, 5.0 mmol) and the reaction mixture was allowed to stir at room temperature overnight. After consumption of the starting material, the reaction mixture was concentrated in vacuo and purified by reversed phase flash chromatography (eluting with 20% ACN in water) to give 311-D (200 mg, 39% yield for two steps) as a white solid. LC/MS (ESI): m/z=479.0 [M+1]⁺. RT=2.027 min

Step-5 The synthesis of 311-E: A suspension of 311-D (200 mg, 0.42 mmol), 4-phenylpicolinamide (130 mg, 0.66 mmol), Cs₂CO₃ (270 mg 0.84 mmol), Pd₂(dba)₃ (92 mg, 0.1 mmol) and Xantphos (60 mg, 0.1 mmol) in dioxane (50 mL) was heated at 100° C. and stirred overnight under nitrogen atmosphere. After cooling to room temperature, water (100 mL) was added and the mixture was extracted with EtOAc (50 mL×2). The combined organic layers were washed with water (30 mL) and brine (30 mL) successively, dried over Na₂SO₄ and concentrated under reduced pressure to give a residue. The residue was purified by chromatography on silica gel (eluting with EA) to give 311-E (150 mg, 60% yield) as a white solid. LC/MS (ESI): m/z=597.1 [M+1]⁺. RT=2.24 min

Step-6 The synthesis of Compound 142: To a solution of 311-E (150 mg, 0.25 mmol) in DCM (25 mL) was added TFA (5.0 mL), and the reaction mixture was stirred at room temperature for 2 h. After consumption of the starting material, the volatiles were evaporated under reduced pressure to give a residue. The resulted residue was dissolved in DCM (50 mL) and basified by aqueous K₂CO₃ (20 mL). The organic layer was separated, washed by brine (30 mL), dried over anhydrous Na₂SO₄, filtered and evaporated under reduced pressure to give a residue. The residue was purified by Prep-HPLC to give Compound 142 (50 mg, 40% yield) as a white solid.

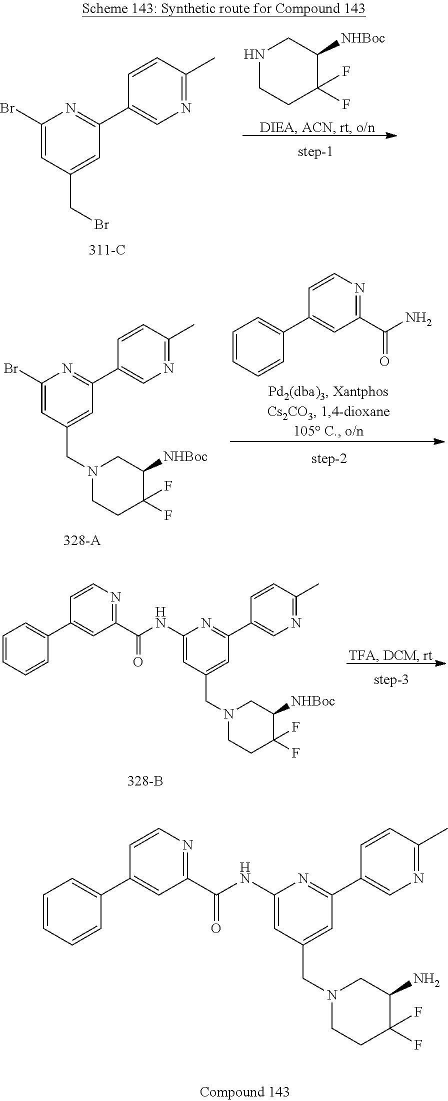

Step-1 The synthesis of 328-A: Crude 311-C (see synthesis of 311-C) was dissolved in ACN (100 mL). To this solution was added DIEA (2.58 g, 20 mmol) and tert-butyl (3R)-(4,4-difluoropiperidin-3-yl)carbamate (1.18 g, 5.0 mmol) and the reaction mixture was allowed to stir at room temperature overnight. After consumption of the starting material, the reaction mixture was concentrated in vacuo and purified by reversed phase flash chromatography on Biotage (eluting with 20% ACN in water) to give 328-A (250 mg, 21% yield for two steps) as a white solid. LC/MS (ESI): m/z=497.0 [M+1]⁺. RT=2.05 min

Step-2 The synthesis of 328-B: A suspension of 328-A (120 mg, 0.24 mmol), 4-phenylpicolinamide (95 mg, 0.48 mmol), Cs₂CO₃ (156 mg 0.48 mmol), Pd₂(dba)₃ (46 mg, 0.05 mmol) and Xantphos (30 mg, 0.05 mmol) in dioxane (50 mL) was heated at 100° C. and stirred overnight under nitrogen atmosphere. After cooling to room temperature, water (100 mL) was added and the mixture was extracted with EtOAc (50 mL×2). The combined organic layers were washed with brine (50 mL), dried over Na₂SO₄ and concentrated under reduced pressure to give a residue. The residue was purified by chromatography on silica gel (eluting with EA) to give 328-B (120 mg, 80% yield) as a white solid. LC/MS (ESI): m/z=615.0 [M+1]⁺. RT=2.27 min

Step-3 The synthesis of Compound 143: To a solution of 328-B (120 mg, 0.20 mmol) in DCM (25 mL) was added TFA (5.0 mL), and the reaction mixture was stirred at room temperature for 2 h. After consumption of the starting material, the volatiles were evaporated under reduced pressure to give a residue. The resulted residue was dissolved in DCM (50 mL) and basified by aqueous K₂CO₃ (20 mL). The organic layer was separated, washed by water (30 mL) and brine (30 mL) successively, dried over anhydrous Na₂SO₄, filtered and evaporated under reduced pressure to give a residue. The residue was purified by Prep-HPLC to give Compound 143 (41 mg, 40% yield) as a white solid.

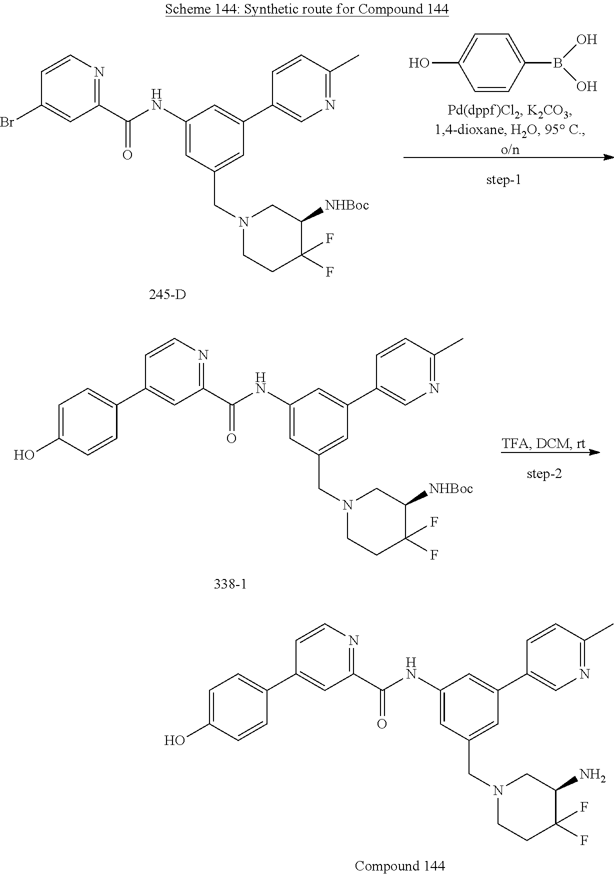 [SMILES: Cc1ccc(-c2cc(CN3CCC(F)(F)[C@H](NC(=O)OC(C)(C)C)C3)cc(NC(=O)c3cc(Br)ccn3)c2)cn1 ; OB(O)c1ccc(O)cc1 ; Cc1ccc(-c2cc(CN3CCC(F)(F)[C@H](NC(=O)OC(C)(C)C)C3)cc(NC(=O)c3cc(-c4ccc(O)cc4)ccn3)c2)cn1 ; Cc1ccc(-c2cc(CN3CCC(F)(F)[C@H](N)C3)cc(NC(=O)c3cc(-c4ccc(O)cc4)ccn3)c2)cn1]

Step-1 The synthesis of 338-1: The title compound 338-1 (120 mg, 0.19 mmol, 38% yield) was prepared according to the General Method F1 with 245-D (200 mg, 0.5 mmol), 4-hydroxyphenylboronic acid (83 mg, 0.60 mmol), K₂CO₃ (207 mg, 1.5 mmol) and Pd(dppf)Cl₂ (37 mg, 0.05 mmol) in 1,4-dioxane (15 mL) and H₂O (3 mL). LC/MS (ESI): m/z=630.0 [M+1]⁺. RT=2.002 min

Step-2 The synthesis of Compound 144: The title compound Compound 144 (99 mg, 99% yield) was prepared according to the General Method G with 338-1 (120 mg, 0.19 mmol) and TFA (3 mL) in DCM (10 mL).

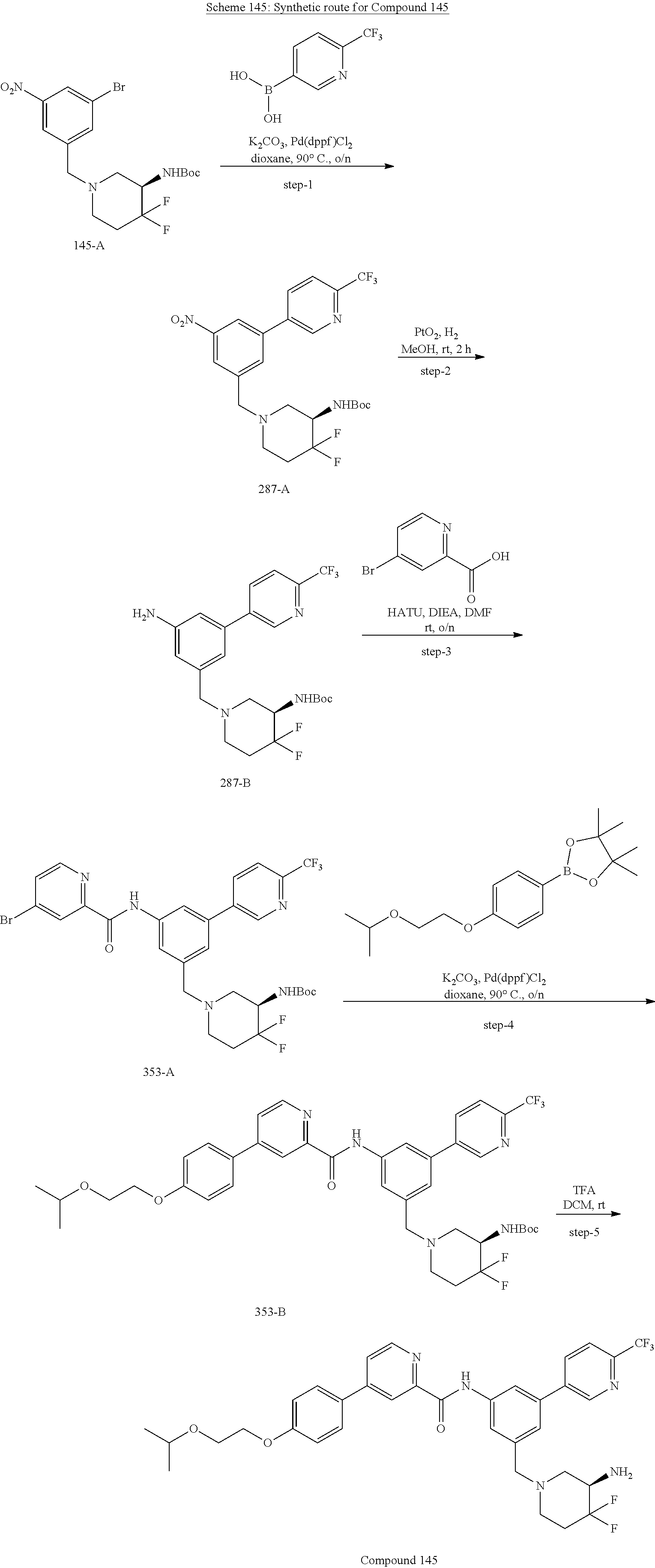

Step-1 The synthesis of 287-A: To a mixture of 145-A (480 mg, 1.06 mmol), 2-(trifluoromethyl)pyridin-5-ylboronic acid (224 mg, 1.2 mmol), and K₂CO₃ (438 mg, 3.18 mmol) in 1, 4-dioxane (30 mL) and H₂O (6 mL) was added Pd(dppf)Cl₂ (78 mg, 0.11 mmol), and the reaction mixture was allowed to heat at reflux and stir overnight under argon atmosphere. After cooling to room temperature, it was diluted by water (100 mL) and extracted with EtOAc (2×50 mL). The organic extract was washed by brine (100 mL), dried over anhydrous Na₂SO₄ and evaporated under reduced pressure to give a residue. The residue was purified by column chromatography on silica gel (eluting with 3% MeOH in DCM) to give 287-A (470 mg, 86% yield) as a white solid. LC/MS (ESI): m/z=517.0 [M+1]⁺. RT=2.148 min

Step-2 The synthesis of 287-B: To a solution of 287-A (470 mg, 0.91 mmol) in MeOH (40 mL) was added PtO₂ (47 mg), and the reaction mixture was stirred at room temperature for 2 hours under hydrogen atmosphere. After consumption of the starting material, the reaction mixture was filtered through a celite pad to remove the solid and the filtrate was evaporated under reduced pressure to give crude 287-B (550 mg, 62% yield) as grey solid. LC/MS (ESI): m/z=487.3 [M+1]⁺. RT=2.242 min

Step-3 The synthesis of 353-A: To a solution of 287-B (200 mg, 0.41 mmol) and 4-bromopicolinic acid (99 mg, 0.49 mmol) in DMF (30 mL) was added HATU (234 mg, 0.62 mmol) and DIEA (159 mg, 1.23 mmol), and the reaction mixture was allowed to stir at room temperature overnight. After consumption of the starting material, the reaction mixture was diluted by water (100 mL) and extracted with EtOAc (2×50 mL). The organic extract was washed by water (4×100 mL) and brine (100 mL) successively, dried over anhydrous Na₂SO₄, filtered and evaporated under reduced pressure to give a residue. The residue was purified by column chromatography on silica gel (eluting with 3% MeOH in DCM) to give 353-A (250 mg, 91% yield) as a white solid. LC/MS (ESI): m/z=670.1 [M+1]⁺. RT=2.553 min

Step-4 The synthesis of 353-B: To a mixture of 353-A (250 mg, 0.37 mmol), 163-D (137 mg, 0.45 mmol), and K₂CO₃ (154 mg, 1.12 mmol) in 1,4-dioxane (30 mL) and H₂O (6 mL) was added Pd(dppf)Cl₂ (30 mg, 0.04 mmol), and the reaction mixture was allowed to heat at reflux and stir overnight under argon atmosphere. After cooling to room temperature, it was diluted by water (100 mL) and extracted with EtOAc (2×50 mL). The organic extract was washed by brine (100 mL), dried over anhydrous Na₂SO₄ and evaporated under reduced pressure to give a residue. The residue was purified by column chromatography on silica gel (eluting with 3% MeOH in DCM) to give 353-B (100 mg, 35% yield) as grey solid. LC/MS (ESI): m/z=770.0 [M+1]⁺. RT=2.349 min

Step-5 The synthesis of Compound 145: To a solution of 353-B (100 mg, 0.13 mmol) in DCM (10 mL) was added TFA (3 mL), and the reaction mixture was stirred at room temperature for 2 h. After consumption of the starting material, the volatiles were evaporated under reduced pressure to give a residue. The resulted residue was dissolved in DCM (50 mL) and basified by aqueous K₂CO₃ (20 mL). The organic layer was separated, washed by water (50 mL) and brine (50 mL) successively, dried over anhydrous Na₂SO₄, filtered and evaporated under reduced pressure, and purified by reversed phase pre-HPLC to give Compound 145 (68 mg, 77% yield) as a white solid.

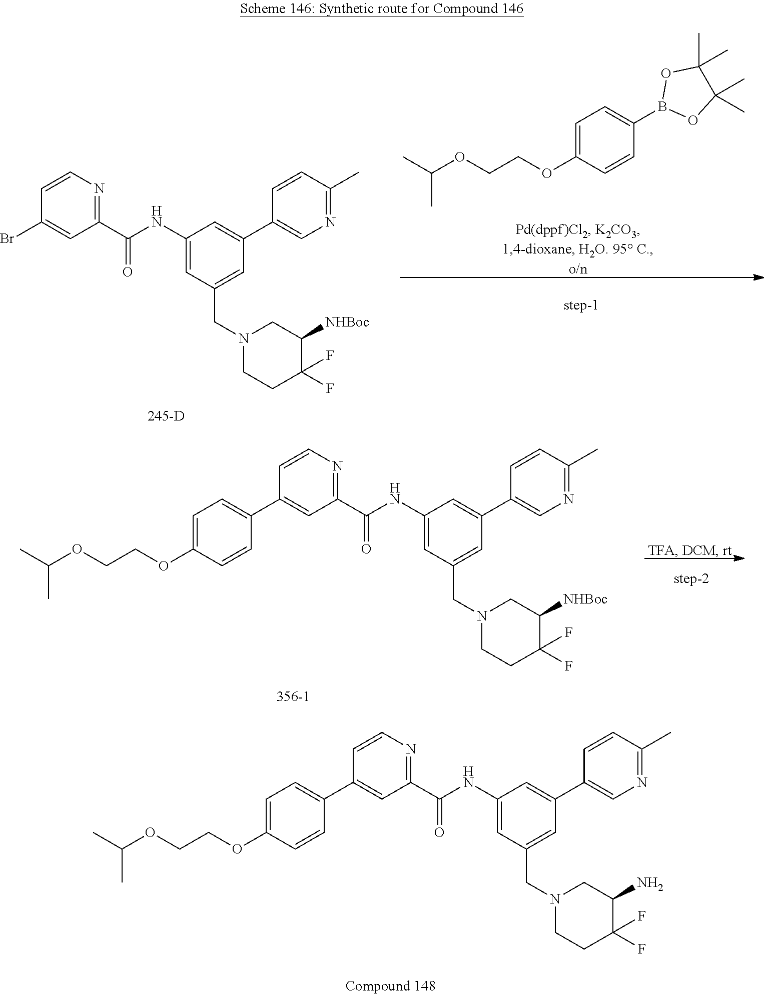

Step-1 The synthesis of 356-1: The title compound 356-1 (132 mg, 0.18 mmol, 56% yield) was prepared according to the General Method F1 with 245-D (200 mg, 0.33 mmol), 2-(4-(2-isopropoxyethoxy)phenyl)-4,4,5,5-tetramethyl-1,3,2-dioxaborolane (119 mg, 0.39 mmol), K₂CO₃ (137 mg, 0.99 mmol) and Pd(dppf)Cl₂ (24 mg, 0.033 mmol) in 1,4-dioxane (15 mL) and H₂O (3 mL). LC/MS (ESI): m/z=716.3 [M+1]⁺. RT=2.12 min

Step-2 The synthesis of Compound 146: The title compound Compound 146 (69 mg, 62% yield) was prepared according to the General Method G with 356-1 (132 mg, 0.18 mmol) and TFA (3 mL) in DCM (10 mL).

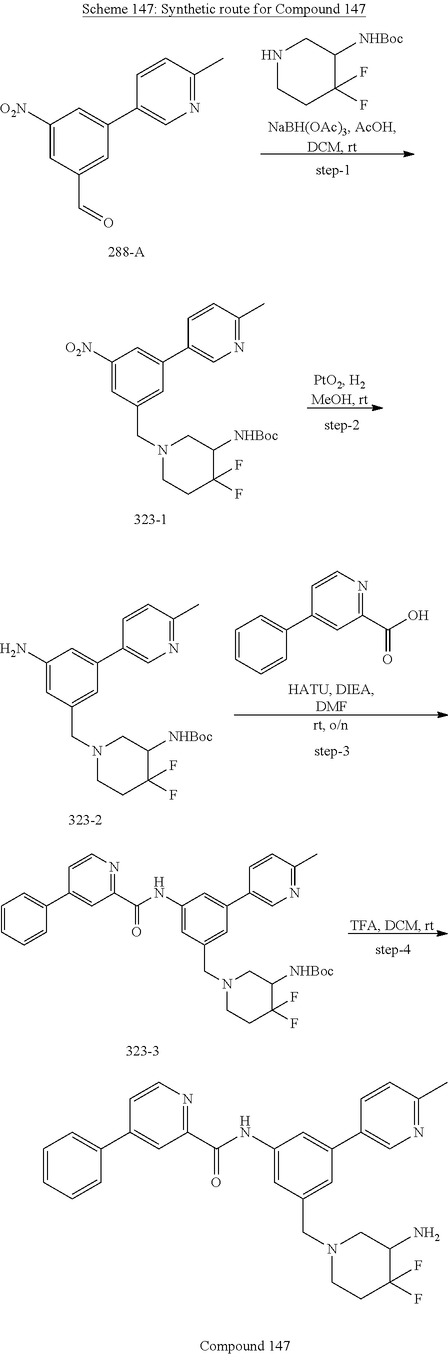

Step-1 The synthesis of 323-1: To a mixture of 288-A (315 mg, 1.3 mmol), tert-butyl (4,4-difluoropiperidin-3-yl)carbamate (236 mg, 1.0 mmol) in DCM (30 mL) was added NaBH(OAc)₃ (635 mg, 3.0 mmol), and the reaction mixture was stirred at room temperature overnight. After consumption of the starting material, it was diluted by water (50 mL), neutralized by aqueous K₂CO₃ and the mixture was extracted with DCM (2×50 mL). The combined organic layers were washed with brine (50 mL), dried over anhydrous Na₂SO₄ and concentrated under reduced pressure to give a residue. The residue was purified by column chromatography on silica gel (eluting with 40% EA in PE) to give 323-1 (280 mg, 61% yield) as a white solid. LC/MS (ESI): m/z=463.3 [M+1]⁺. RT=2.23 min

Step-2 The synthesis of 323-2: To a solution of 323-1 (280 mg, 0.61 mmol) in MeOH (40 mL) was added PtO₂ (30 mg), and the reaction mixture was stirred at room temperature for 3 h under hydrogen atmosphere. After consumption of the starting material, the reaction mixture was filtered through a celite pad to remove the solid and the filtrate was evaporated under reduced pressure to give crude 323-2 (250 mg, 96% yield) as colorless solid. LC/MS (ESI): m/z=433.3 [M+1]⁺. RT=2.01 min

Step-3 The synthesis of 323-3: To a solution of 323-2 (250 mg, 0.58 mmol) and 4-phenylpicolinic acid (165 mg, 0.81 mmol) in DMF (25 mL) was added HATU (330 mg, 0.87 mmol) and DIEA (224 mg, 1.74 mmol), and the reaction mixture was allowed to stir at room temperature overnight. After consumption of the starting material, the reaction mixture was diluted by water (50 mL) and extracted with EtOAc (2×50 mL). The organic extract was washed by water (4×100 mL) and brine (50 mL) successively, dried over anhydrous Na₂SO₄, filtered and evaporated under reduced pressure to give a residue. The residue was purified by column chromatography on silica gel (eluting with 50% EA in PE) to give 323-3 (285 mg, 38% yield) as colorless gel. LC/MS (ESI): m/z=614.3 [M+1]⁺. RT=2.47 min

Step-4 The synthesis of Compound 147: To a solution of 323-3 (285 mg, 0.46 mmol) in DCM (25 mL) was added TFA (5 mL), and the reaction mixture was stirred at room temperature for 2 h. After consumption of the starting material, the volatiles were evaporated under reduced pressure to give a residue. The resulted residue was dissolved in DCM (30 mL) and basified by aqueous K₂CO₃ (30 mL). The organic layer was separated, washed by water (30 mL) and brine (30 mL) successively, dried over anhydrous Na₂SO₄, filtered, evaporated under reduced pressure and purified by Prep-HPLC to give Compound 147 (165 mg, 69% yield) as an off-white solid.

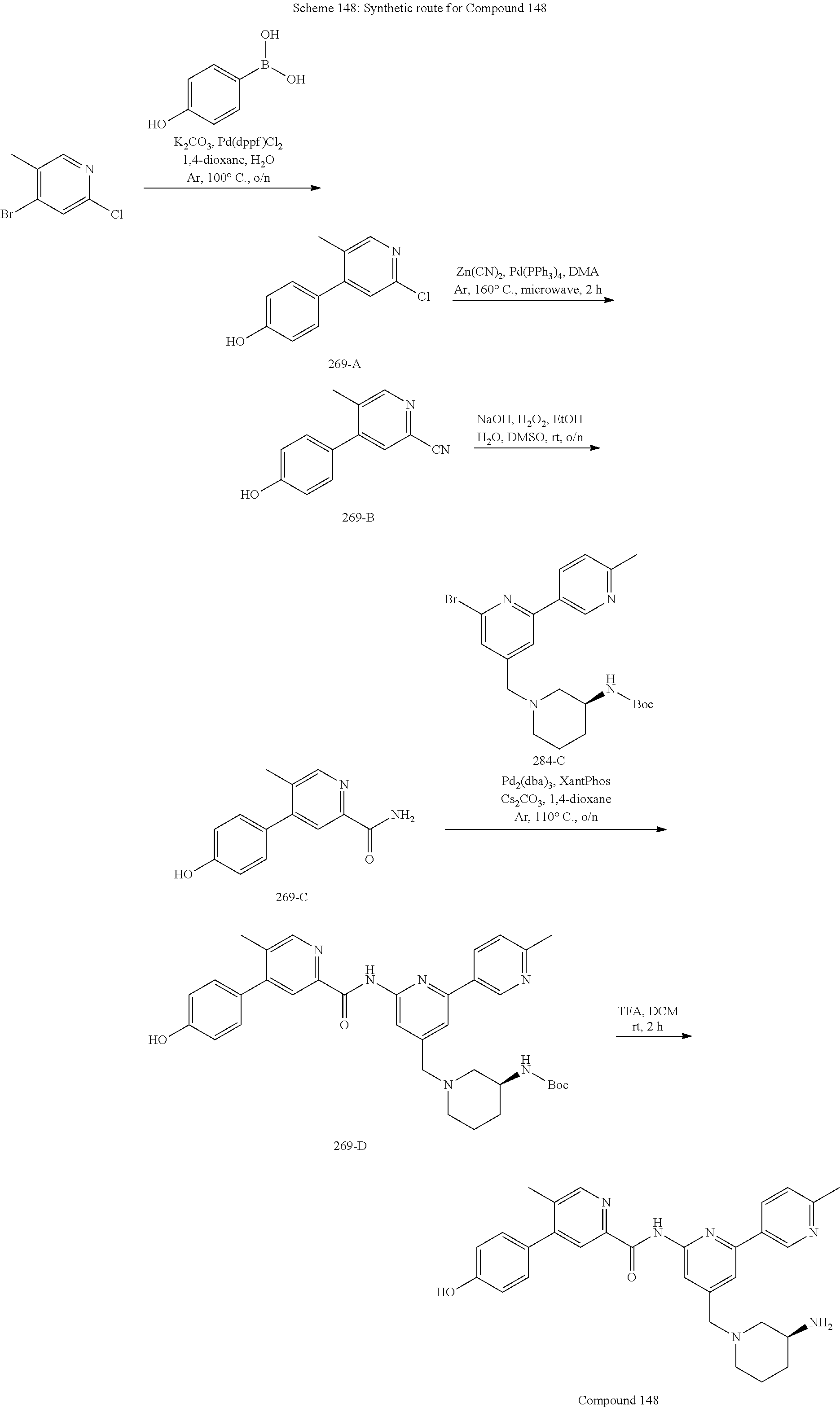

Step-1 The synthesis of 269-A: A mixture of (4-hydroxyphenyl)boronic acid (668 mg, 4.84 mmol), 4-bromo-2-chloro-5-methylpyridine (1.0 g, 4.84 mmol), K₂CO₃ (2.0 g, 14.5 mmol) and Pd(dppf)Cl₂ (351 mg, 0.48 mmol) in 1, 4-dioxane (80 mL) and H₂O (16 mL) was heated at 100° C. and stirred overnight under argon. After cooling to room temperature, water was added and the mixture was extracted with EtOAc (50 mL×2). The combined organic layers were washed with brine (50 mL), dried over Na₂SO₄ and concentrated. The residue was purified by silica gel column chromatography (eluting with 16% EA in PE) to give 269-A (745 mg, 44% yield) as a white solid. LC/MS (ESI): m/z=220.1 [M+1]⁺. RT=1.689 min

Step-2 The synthesis of 269-B: A mixture of 269-A (300 mg, 1.36 mmol), Zn(CN)₂ (321 mg, 2.73 mmol) and Pd(PPh₃)₄ (631 mg, 0.55 mmol) in DMA (10 mL) was heated at 160° C. and stirred for 2 hours under argon with microwave. After cooling to room temperature, water (50 mL) was added and the mixture was extracted with EtOAc (50 mL×2). The combined organic layers were washed with brine (50 mL×4), dried over Na₂SO₄ and concentrated to give crude 269-B (287 mg) as a brown gel. LC/MS (ESI): m/z=211.1 [M+1]⁺. RT=1.589 min

Step-3 The synthesis of 269-C: To a solution of 269-B (287 mg, 1.36 mmol) and NaOH (55 mg, 1.36 mmol) in EtOH (20 mL), H₂O (2 mL) and DMSO (40 mL) was added H₂O₂ (9.6 mL, 9.6 mmol), and the reaction mixture was stirred at room temperature overnight. After consumption of the starting material, the volatiles were evaporated under reduced pressure to give a residue. The resulted residue was added with water (30 mL) and extracted with EtOAc (30 mL×2). The combined organic layers were washed with brine (50 mL×4), dried over Na₂SO₄ and concentrated. The residue was purified by silica gel column chromatography (eluting with 50% EA in PE) to give 269-C (300 mg, 96% yield) as a white solid. LC/MS (ESI): m/z=229.3 [M+1]⁺. RT=1.405 min

Step-4 The synthesis of 269-D: A mixture of 269-C (119 mg, 0.52 mmol), 284-C (120 mg, 0.26 mmol), Pd₂(dba)₃ (24 mg, 0.03 mmol), XantPhos (30 mg, 0.05 mmol) and Cs₂CO₃ (254 mg, 0.78 mmol) in 1, 4-dioxane (20 mL) was heated at 110° C. and stirred overnight under argon. After cooling to room temperature, water (50 mL) was added and the mixture was extracted with EtOAc (50 mL) for three times, the combined organic layers were washed with brine (50 mL) for four times, dried over Na₂SO₄ and concentrated. The residue was purified by silica gel column chromatography (eluting with 75% EA in PE) to give 269-D (80 mg, 50% yield) as a white solid. LC/MS (ESI): m/z=609.1 [M+1]⁺. RT=2.102 min

Step-5 The synthesis of Compound 148: To a solution of 269-D (80 mg, 0.13 mmol) in DCM (20 mL) was added TFA (2 mL), and the reaction mixture was stirred at room temperature for 2 h. After consumption of the starting material, the volatiles were evaporated under reduced pressure to give a residue. The resulted residue was dissolved in DCM (30 mL) and basified by aqueous K₂CO₃ (30 mL). The organic layer was separated, washed by water (30 mL) and brine (30 mL) successively, dried over anhydrous Na₂SO₄, filtered, evaporated under reduced pressure and purified by Prep-HPLC to give Compound 148 (5 mg, 7.4% yield) as a white solid.

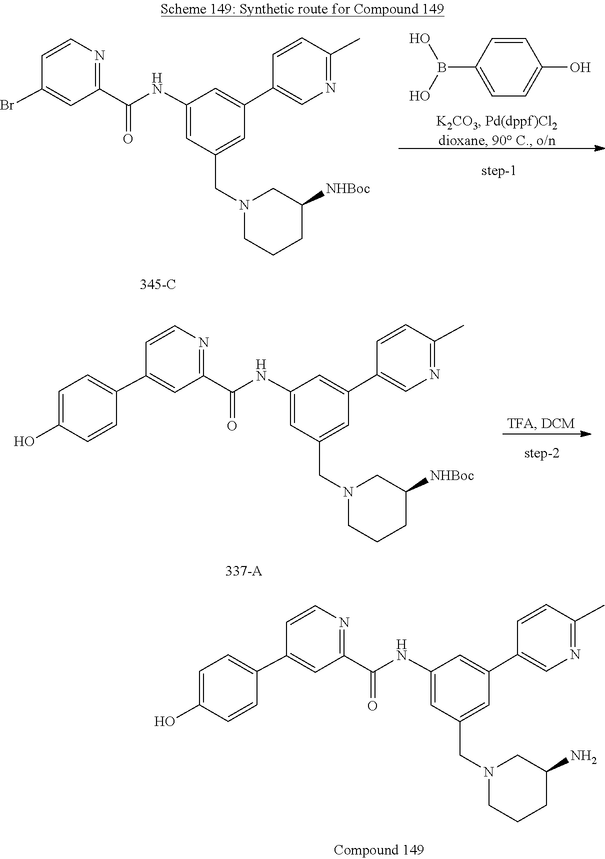 [SMILES: Cc1ccc(-c2cc(CN3CCC[C@H](NC(=O)OC(C)(C)C)C3)cc(NC(=O)c3cc(Br)ccn3)c2)cn1 ; OB(O)c1ccc(O)cc1 ; Cc1ccc(-c2cc(CN3CCC[C@H](NC(=O)OC(C)(C)C)C3)cc(NC(=O)c3cc(-c4ccc(O)cc4)ccn3)c2)cn1 ; Cc1ccc(-c2cc(CN3CCC[C@H](N)C3)cc(NC(=O)c3cc(-c4ccc(O)cc4)ccn3)c2)cn1]

Step-1 The synthesis of 337-A: To a mixture of 345-C (200 mg, 0.34 mmol) and 4-hydroxyphenylboronic acid (61 mg, 0.44 mmol) in 1,4-dioxane/H₂O (15 mL/3 mL) was added Pd(dppf)Cl₂ (25 mg, 0.034 mmol) and K₂CO₃ (141 mg, 1.02 mmol), then the reaction mixture was stirred at 90° C. overnight under Ar atmosphere. After consumption of the starting material, water (30 mL) was added and the mixture was extracted with EA (2×50 mL). The combined organic layers were washed with brine (50 mL), dried over anhydrous Na₂SO₄ and concentrated in vacuo to give a residue. The residue was purified by column chromatography on silica gel (eluting with DCM:MeOH=25:1) to give 337-A (170 mg, 73% yield) as a yellow liquid. LC/MS (ESI): m/z=594.2 [M+1]⁺. RT=1.82 min

Step-2 The synthesis of Compound 149: To a solution of 337-A (210 mg, 0.35 mmol) in DCM (15 mL) was added TFA (5 mL). The reaction was stirred at room temperature for 2 h, and the volatiles were evaporated under reduced pressure. The residue was treated with water (20 mL) and basified with aqueous K₂CO₃, then extracted with DCM (3×15 mL). The organic extract was washed with brine (50 mL), dried over anhydrous Na₂SO₄, filtered and evaporated under reduced pressure to give a residue. The residue was purified by reversed phase Prep-HPLC to give Compound 149 (20 mg, 19% yield) as a white solid.

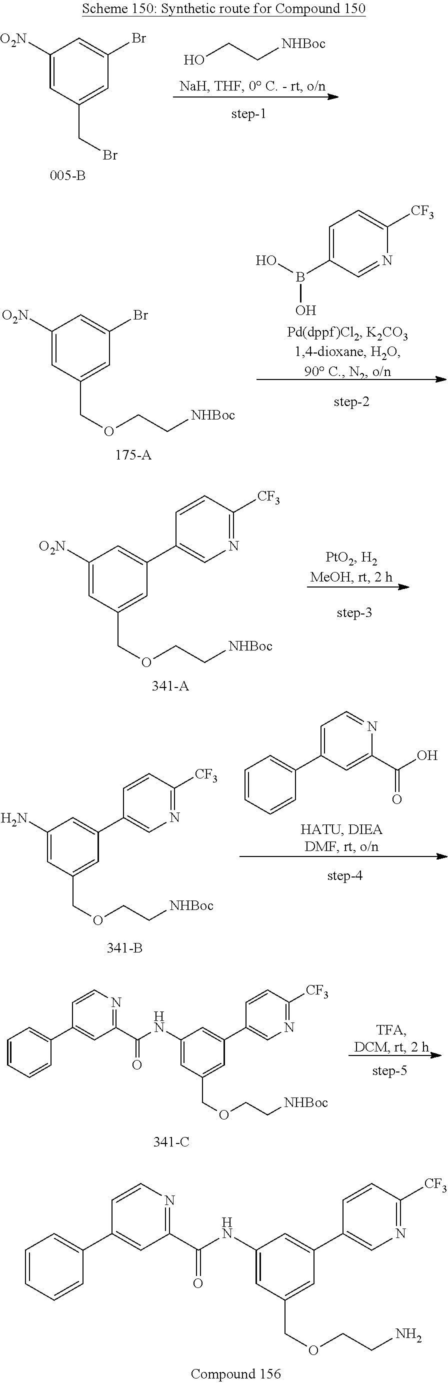

Step-1 The synthesis of 175-A: To a mixture of tert-butyl 2-hydroxyethylcarbamate (483 mg, 3.0 mmol) in dry THF (50 mL) was added NaH (60% wt, 384 mg, 9.6 mmol) at 0° C. After the reaction mixture was stirred at 0° C. for 30 min, a solution of 005-B (590 mg, 2.0 mmol) in dry THF (5 mL) was added, and the reaction mixture was allowed to stir at room temperature overnight. After consumption of the starting material, water (100 mL) was added to quench the reaction and the mixture was extracted with EtOAc (2×50 mL). The combined organic layers were washed with brine (100 mL), dried over anhydrous Na₂SO₄ and concentrated under reduced pressure to give a residue. The residue was purified by column chromatography on silica gel (eluting with 15% EA in PE) to give 175-A (200 mg, 27% yield) as a yellow solid. LC/MS (ESI): m/z=319.0 [M+1]⁺. RT=2.026 min

Step-2 The synthesis of 341-A: To a mixture of 175-A (250 mg, 0.66 mmol), 2-(trifluoromethyl)pyridin-5-ylboronic acid (152 mg, 0.80 mmol), and K₂CO₃ (276 mg, 2.0 mmol) in 1, 4-dioxane (30 mL) and H₂O (6 mL) was added Pd(dppf)Cl₂ (50 mg, 0.07 mmol), and the reaction mixture was allowed to heat at reflux and stir overnight under nitrogen atmosphere. After cooling to room temperature, it was diluted by water (100 mL) and extracted with EtOAc (2×50 mL). The organic extract was washed by brine (100 mL), dried over anhydrous Na₂SO₄ and evaporated under reduced pressure to give a residue. The residue was purified by column chromatography on silica gel (eluting with 3% MeOH in DCM) to give 341-A (110 mg, 38% yield) as a yellow oil. LC/MS (ESI): m/z=386.1 [M+1]⁺. RT=2.272 min

Step-3 The synthesis of 341-B: To a solution of 341-A (110 mg, 0.25 mmol) in MeOH (20 mL) was added PtO₂ (11 mg), and the reaction mixture was stirred at room temperature for 2 hours under hydrogen atmosphere. After consumption of the starting material, the reaction mixture was filtered through a celite pad to remove the solid and the filtrate was evaporated under reduced pressure to give crude 241-B (80 mg, 78% yield) as a black solid. LC/MS (ESI): m/z=356.2 [M+1]⁺. RT=2.097 min

Step-4 The synthesis of 341-C: To a solution of 341-B (80 mg, 0.19 mmol) and 4-phenylpicolinic acid (46 mg, 0.23 mmol) in DMF (20 mL) was added HATU (108 mg, 0.29 mmol) and DIEA (74 mg, 0.57 mmol), and the reaction mixture was allowed to stir at room temperature overnight. After consumption of the starting material, the reaction mixture was diluted by water (50 mL) and extracted with EtOAc (2×50 mL). The organic extract was washed by water (4×100 mL) and brine (100 mL) successively, dried over anhydrous Na₂SO₄, filtered and evaporated under reduced pressure to give a residue. The residue was purified by column chromatography on silica gel (eluting with 3% MeOH in DCM) to give 341-C (110 mg, 97% yield) as a yellow solid. LC/MS (ESI): m/z=537.0 [M+1]⁺. RT=2.238 min

Step-5 The synthesis of Compound 150: To a solution of 341-C (110 mg, 0.18 mmol) in DCM (10 mL) was added TFA (3 mL), and the reaction mixture was stirred at room temperature for 2 h. After consumption of the starting material, the volatiles were evaporated under reduced pressure to give a residue. The resulted residue was dissolved in DCM (30 mL) and basified by aqueous K₂CO₃ (20 mL). The organic layer was separated, washed by water (30 mL) and brine (30 mL) successively, dried over anhydrous Na₂SO₄, filtered and evaporated under reduced pressure to give Compound 150 (65 mg, 71% yield) as a white solid.

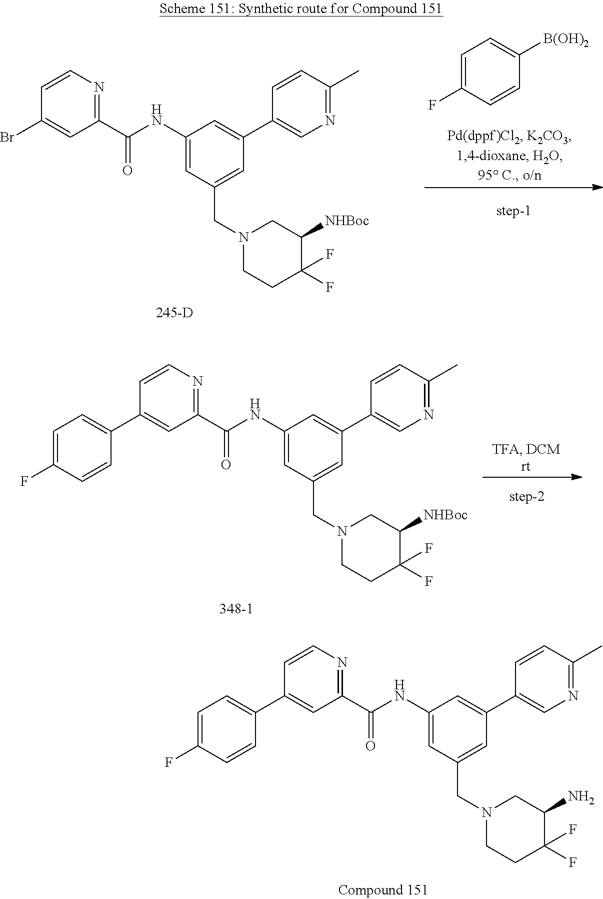 [SMILES: Cc1ccc(-c2cc(CN3CCC(F)(F)[C@H](NC(=O)OC(C)(C)C)C3)cc(NC(=O)c3cc(Br)ccn3)c2)cn1 ; OB(O)c1ccc(F)cc1 ; Cc1ccc(-c2cc(CN3CCC(F)(F)[C@H](NC(=O)OC(C)(C)C)C3)cc(NC(=O)c3cc(-c4ccc(F)cc4)ccn3)c2)cn1 ; Cc1ccc(-c2cc(CN3CCC(F)(F)[C@H](N)C3)cc(NC(=O)c3cc(-c4ccc(F)cc4)ccn3)c2)cn1]

Step-1 The synthesis of 348-1: The title compound 348-1 (157 mg, 0.25 mmol, 75% yield) was prepared according to the General Method F1 with 245-D (200 mg, 0.33 mmol), 4-fluorophenylboronic acid (55 mg, 0.39 mmol), K₂CO₃ (137 mg, 0.99 mmol) and Pd(dppf)Cl₂ (24 mg, 0.033 mmol) in 1,4-dioxane (15 mL) and H₂O (3 mL). LC/MS (ESI): m/z=632.2 [M+1]⁺. RT=2.05 min

Step-2 The synthesis of Compound 151: The title compound Compound 151 (88 mg, 66% yield) was prepared according to the General Method G with 348-1 (157 mg, 0.25 mmol) and TFA (3 mL) in DCM (10 mL).

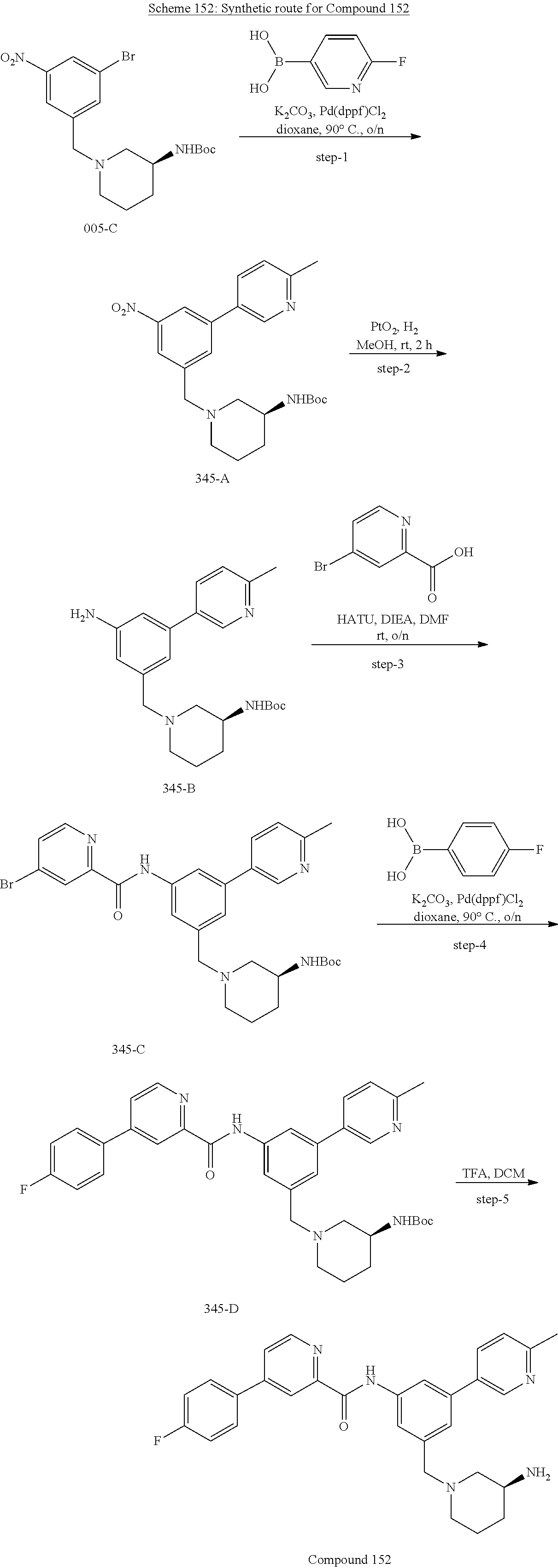

Step-1 The synthesis of 345-A: To a mixture of 005-C (2.50 g, 6.03 mmol) and 2-methylpyridin-5-ylboronic acid (1.07 g, 7.84 mmol) in 1,4-dioxane/H₂O (40 mL/8 mL) was added Pd(dppf)Cl₂ (440 mg, 0.60 mmol) and K₂CO₃ (2.50 g, 18.09 mmol). The reaction mixture was stirred at 90° C. for 48 h under Ar atmosphere. After consumption of the starting material, water (50 mL) was added and the mixture was extracted with EA (2×50 mL). The combined organic layers were washed with brine (50 mL), dried over anhydrous Na₂SO₄ and concentrated in vacuo to give the residue. The residue was purified by column chromatography on silica gel (eluting with PE:EA=1:1) to give 345-A (1.60 g, 62% yield) as a yellow solid. LC/MS (ESI): m/z=427.1 [M+1]⁺. RT=2.00 min

Step-2 The synthesis of 345-B: To a solution of 345-A (1.60 g, 3.75 mmol) in MeOH (40 mL) was added PtO₂ (80 mg). The reaction mixture was stirred at room temperature for 2 h under H₂. After consumption of the starting material, the mixture was filtered through a celite pad and concentrated in vacuo to give crude 345-B (1.48 g) as black solid. LC/MS (ESI): m/z=397.1 [M+1]⁺. RT=1.758 min

Step-3 The synthesis of 345-C: To a solution of 345-B (1.48 g, 3.75 mmol) and 4-bromopicolinic acid (990 mg, 4.91 mmol) in DMF (30 mL) was added HATU (2.15 g, 5.67 mmol) and DIEA (2 mL). The reaction mixture was stirred at room temperature overnight. After consumption of the starting material, water (60 mL) was added and the mixture was extracted with EA (3×60 mL). The combined organic layers were washed with water (4×100 mL) and brine (100 mL) successively, dried over anhydrous Na₂SO₄ and concentrated in vacuo to give a residue. The residue was purified by column chromatography on silica gel (eluting with PE:EA=1:2) to give 345-C (1.90 g, 86% yield) as a yellow solid. LC/MS (ESI): m/z=580.0 [M+1]⁺. RT=2.128 min

Step-4 The synthesis of 345-D: To a mixture of 345-C (340 mg, 0.58 mmol) and 4-fluorophenylboronic acid (106 mg, 0.76 mmol) in 1,4-dioxane/H₂O (20 mL/4 mL) was added Pd(dppf)Cl₂ (42 mg, 0.058 mmol) and K₂CO₃ (240 mg, 1.74 mmol). The reaction mixture was stirred at 90° C. overnight under Ar atmosphere. After consumption of the starting material, water (50 mL) was added and the mixture was extracted with EA (2×50 mL). The combined organic layers were washed with brine (50 mL), dried over anhydrous Na₂SO₄ and concentrated in vacuo to give a residue. The residue was purified by column chromatography on silica gel (eluting with PE:EA=1:2) to give 345-D (180 mg, 51% yield) as a yellow solid. LC/MS (ESI): m/z=596.0 [M+1]⁺. RT=2.202 min

Step-5 The synthesis of Compound 152: To a solution of 345-D (180 mg, 0.3 mmol) in DCM (15 mL) was added TFA (5 mL). The reaction was stirred at room temperature for 2 h before evaporation under reduce pressure. The residue was treated with water (20 mL) and basified with aqueous K₂CO₃, then extracted with DCM (3×15 mL). The organic extract was washed with brine (30 mL), dried over anhydrous Na₂SO₄, filtered and evaporated under reduced pressure to give a residue. The residue was purified by Prep-HPLC to give Compound 152 (40 mg, 26% yield) as a white solid.

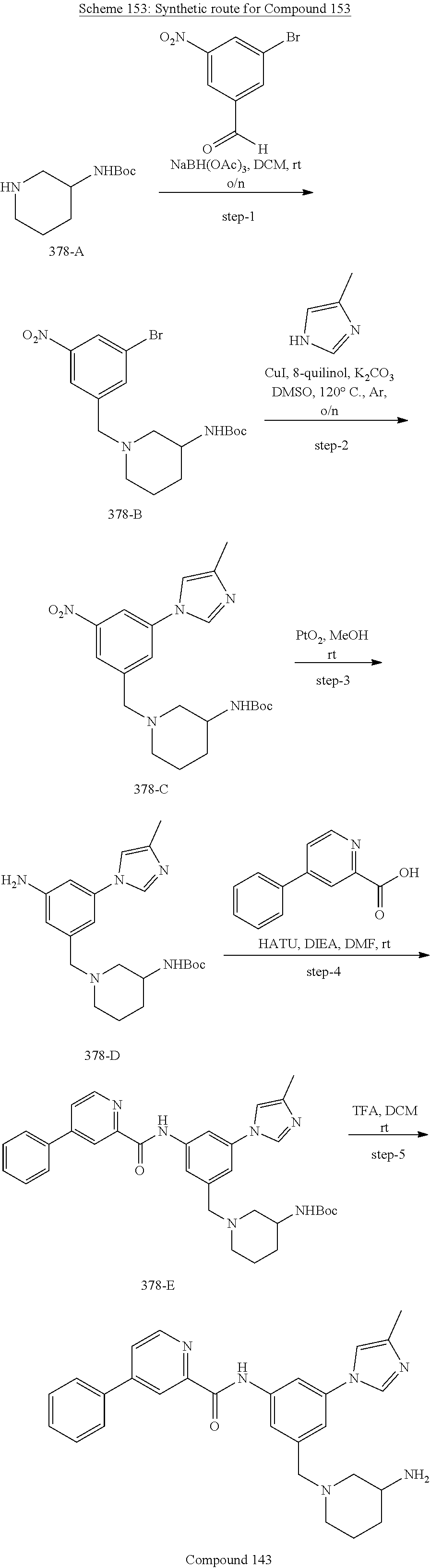

Step-1 The synthesis of 378-B: To a mixture of 378-A (600 mg, 3 mmol), 3-bromo-5-nitrobenzaldehyde (1.0 g, 4.5 mmol) in DCM (20 mL) was added NaBH(OAc)₃ (2 g, 9 mmol), and the reaction mixture was stirred at room temperature overnight. After consumption of the starting material, water (50 mL) was added and the mixture was extracted with EtOAc (2×50 mL). The combined organic layers were washed with water (4×50 mL) and brine (50 mL) successively, dried over anhydrous Na₂SO₄ and concentrated under reduced pressure to give a residue. The residue was purified by column chromatography on silica gel (eluting with 20% EA in PE) to give 378-B (950 mg, 76% yield) as a yellow solid. LC/MS (ESI): m/z=414.0 [M+1]⁺. RT=1.96 min

Step-2 The synthesis of 378-C: The title compound 317-C (478 mg, 1.15 mmol, 50% yield) was prepared according to the General Method C with 317-B (950 mg, 2.3 mmol), 4-methyl-1H-imidazole (566 mg, 6.9 mmol), K₂CO₃ (952 mg, 6.9 mmol) and CuI (131 mg, 0.69 mmol), 8-quilinol (67 mg, 0.46 mmol) in DMSO (15 mL). LC/MS (ESI): m/z=416.2 [M+1]⁺. RT=1.72 min

Step-3 The synthesis of 378-D: The title compound 317-D (365 mg, 0.95 mmol, 83% yield) was prepared according to the General Method D1 with 317-C (478 mg, 1.15 mmol), PtO₂ (50 mg) in MeOH (30 mL). LC/MS (ESI): m/z=386.2 [M+1]⁺. RT=1.46 min

Step-4 The synthesis of 378-E: The title compound 378-E (110 mg, 0.19 mmol, 45% yield) was prepared according to the General Method E with 378-D (160 mg, 0.42 mmol), 4-phenylpicolinic acid (116 mg, 0.58 mmol), HATU (239 mg, 0.63 mmol) and DIEA (163 mg, 1.26 mmol) in DMF (15 mL). LC/MS (ESI): m/z=567.2 [M+1]⁺. RT=1.94 min

Step-5 The synthesis of Compound 153: The title compound Compound 153 (22 mg, 24.8% yield) was prepared according to the General Method G with 348-E (110 mg, 0.19 mmol) and TFA (3 mL) in DCM (10 mL).

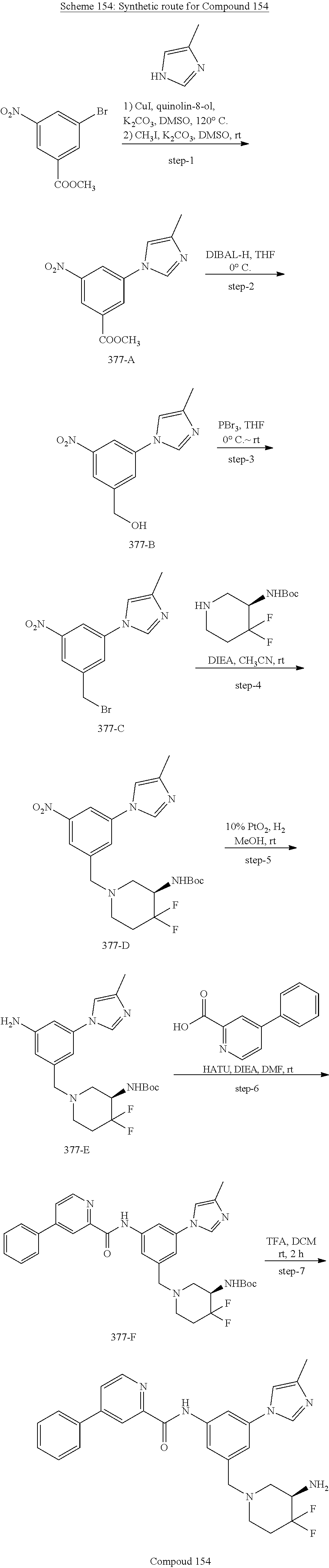

Step-1 The synthesis of 377-A: A suspension of methyl 3-bromo-5-nitrobenzoate (2.60 g, 10.0 mmol), 4-methyl-1H-imidazole (1.23 g, 15.0 mmol), K₂CO₃ (2.76 mg, 20.0 mmol), CuI (570 mg, 3.0 mmol) and quinolin-8-ol (290 mg, 2.0 mmol) in DMSO (70 mL) was heated at 120° C. overnight under nitrogen atmosphere. After cooling to room temperature, the mixture was added with CH₃₁ (0.75 mL, 12.0 mmol), stirred for 2 h at room temperature, diluted by water (100 mL) and extracted with EtOAc (2×100 mL). The combined organic layers were washed with water (4×200 mL) and brine (100 mL) successively, dried over Na₂SO₄ and concentrated under reduced pressure to give a residue. The residue was purified by column chromatography on silica gel (eluting with 60% EA in PE) to give 377-A (830 mg, 32% yield) as a yellow solid. LC/MS (ESI): m/z=262.3 [M+1]⁺. RT=1.75 min

Step-2 The synthesis of 377-B: To a solution of 377-A (400 mg, 1.53 mmol) in dry THF (30 mL) was added DIBAL-H (4.6 mL, 4.6 mmol, 1.0 M in hexane) at 0° C., and the reaction mixture was allowed to warm to room temperature and stirred overnight. After consumption of the starting material, it was quenched by 10% aqueous NaOH, concentrated, and the mixture was extracted with EtOAc (2×50 mL). The combined organic layers were washed with brine (50 mL), dried over anhydrous Na₂SO₄ and concentrated under reduced pressure to give crude 377-B (340 mg, 95% yield) as a yellow semi-solid. LC/MS (ESI): m/z=234.2 [M+1]⁺. RT=1.46 min

Step-3 The synthesis of 377-C: To a solution of 377-B (1.0 g, 3.43 mmol) in THF (30 mL) was added PBr₃ (590 mg, 2.19 mmol) at 0° C., and the reaction mixture was stirred at room temperature overnight. After consumption of the starting material, it was concentrated to remove THF to give crude 377-C (430 mg, 99% yield) as a yellow gel. LC/MS (ESI): m/z=296.1 [M+1]⁺. RT=1.88 min

Step-4 The synthesis of 377-D: To a mixture of 377-C (210 mg, 0.71 mmol), tert-butyl (3R)-(4,4-difluoropiperidin-3-yl)carbamate (140 mg, 0.59 mmol) in CH₃CN (30 mL) was added DIEA (2.5 mL), and the reaction mixture was stirred at room temperature overnight. After consumption of the starting material, it was concentrated to remove CH₃CN, water (30 mL) was added and the mixture was extracted with EtOAc (2×30 mL). The combined organic layers were washed with brine (50 mL), dried over anhydrous Na₂SO₄ and concentrated under reduced pressure to give a residue. The residue was purified by column chromatography on silica gel (eluting with 80% EA in PE) to give 377-D (120 mg, 45% yield) as a light-yellow solid. LC/MS (ESI): m/z=452.3 [M+1]⁺. RT=2.10 min

Step-5 The synthesis of 377-E: To a solution of 377-D (120 mg, 0.27 mmol) in MeOH (20 mL) was added PtO₂ (20 mg), and the reaction mixture was stirred at room temperature for 3 h under hydrogen atmosphere. After consumption of the starting material, the reaction mixture was filtered through a celite pad to remove the solid and the filtrate was evaporated under reduced pressure to give crude 377-E (110 mg, 99% yield) as a brown solid. LC/MS (ESI): m/z=422.4 [M+1]⁺. RT=1.90 min

Step-5 The synthesis of 377-F: To a solution of 377-E (110 mg, 0.26 mmol) and 4-phenylpicolinic acid (52 mg, 0.26 mmol) in DMF (15 mL) was added HATU (120 mg, 0.31 mmol) and DIEA (0.5 mL), and the reaction mixture was allowed to stir at room temperature overnight. After consumption of the starting material, the reaction mixture was diluted by water (30 mL) and extracted with EtOAc (2×30 mL). The organic extract was washed by water (4×50 mL) and brine (30 mL) successively, dried over anhydrous Na₂SO₄, filtered and evaporated under reduced pressure to give a residue. The residue was purified by column chromatography on silica gel (eluting with 80% EA in PE) to give 377-F (56 mg, 35% yield) as a light-yellow solid. LC/MS (ESI): m/z=603.4 [M+1]⁺. RT=2.36 min

Step-6 The synthesis of Compound 154: To a solution of 377-F (56 mg, 0.09 mmol) in DCM (15 mL) was added TFA (2.5 mL), and the reaction mixture was stirred at room temperature for 2 h. After consumption of the starting material, the volatiles were evaporated under reduced pressure to give a residue. The resulted residue was dissolved in DCM (20 mL) and basified by aqueous K₂CO₃ (20 mL). The organic layer was separated, washed by water (20 mL) and brine (20 mL) successively, dried over anhydrous Na₂SO₄, filtered, evaporated under reduced pressure and purified by Prep-HPLC to give Compound 154 (13 mg, 28% yield) as a white solid.

The analytical data for the synthesised compounds is found in the table 4 below:

TABLE 4

| Compound | |
|---|---|
| number | Analytical data |
| 1 | LC/MS: m/z 490.46 (M + H)⁺ (ES+), 99.72% |
| | ¹H NMR (400 MHZ, DMSO-d₆): δ 1.60-1.70 (m, 1H), 2.00-2.10 (m, 2H), 2.17 (s, 3H), |
| | 2.20 (s, 3H), 2.36 (s, 3H), 2.50-2.59 (m, 2H), 2.60-2.70 (m, 2H), 3.30-3.40 (m, 1H), |
| | 3.60-3.70 (m, 2H), 3.80-4.00 (m, 5H), 5.10-5.20 (m, 1H), 7.26 (s, 1H), 7.31 (d, J = 7.8 |
| | Hz, 1H), 7.35 (s, 1H), 7.47 (s, 1H), 7.50 (d, J = 7.6 Hz, 1H), 7.67 (s, 1H), 7.93 (s, 1H), |
| | 8.04 (s, 1H), 8.30 (bs, 1H), 10.35 (bs, 1H) |
| 2 | LC/MS: m/z 488.41 (M + H)⁺ (ES+), 99.66% |
| | ¹H NMR (400 MHZ, DMSO-d₆): δ 1.32-1.49 (m, 2H), 1.60-1.70 (m, 2H), 1.80-2.00 (m, |
| | 3H), 2.10-2.20 (m, 2H), 2.33-2.40 (m, 2H), 2.31 (s, 3H), 2.57 (s, 3H), 3.00-3.20 (m, |
| | 4H), 3.30-3.40 (m, 1H), 3.78-3.82 (m, 1H), 4.10-4.20 (m, 2H), 4.25-4.35 (m, 1H), 6.85- |
| | 6.97 (m, 3H), 7.20-7.30 (m, 2H), 7.41 (s, 1H), 7.75 (s, 1H), 7.80 (s, 1H), 7.86 (s, 1H), |
| | 9.18 (s, 1H) |
| 7 | LC/MS: m/z 489.30 (M + H)⁺ (ES+), 99.12% |
| | ¹H NMR (400 MHZ, DMSO-d6): δ 1.42-1.51 (m, 1H), 1.53-1.62 (m, 2H), 1.90-2.00 (m, |
| | 3H), 2.15 (s, 3H), 2.20 (s, 3H), 2.20-2.25 (m, 1H), 2.40-2.50 (m, 2H), 2.65-2.73 (m, |
| | 1H), 3.02-3.10 (m, 1H), 3.30-3.32 (m, 2H), 3.40-3.45 (m, 2H), 3.60-3.70 (m, 2H), 4.58- |
| | 4.65 (m, 1H), 6.91 (t, J = 7.3 Hz, 1H), 6.98 (d, J = 8.1 Hz, 2H), 7.04 (s, 1H), 7.26-7.31 |
| | (m, 3H), 7.36 (s, 1H), 7.62 (s, 1H), 7.96 (s, 1H), 8.75 (s, 1H) |
| 8 | LC/MS: m/z 475.31 (M + H)⁺ (ES+), 98.13% |
| | ¹H NMR (400 MHZ, DMSO-d₆): δ 1.40-1.50 (m, 1H), 1.98-2.03 (m, 2H), 2.15 (s, 3H), |
| | 2.22 (s, 3H), 2.40-2.60 (m, 2H), 2.70-2 .73 (m, 1H), 3.00-3.10 (m, 1H), 3.45-3.55 (m, |
| | 4H), 3.60-3.72 (m, 4H), 4.99-5.13 (m, 1H), 6.95-7.00 (m, 3H), 7.03 (s, 1H), 7.28-7.35 |
| | (m, 3H), 7.40 (s, 1H), 7.67 (s, 1H), 7.94 (s, 1H), 8.40 (s, 1H) |
| 9 | LC/MS: m/z 503.29 (M + H)⁺ (ES+), 98.40% |
| | ¹H NMR (400 MHZ, DMSO-d₆): δ 1.40-1.50 (m, 1H), 1.66-1.72 (m, 1H), 1.80-1.98 (m, |
| | 8H), 2.00-2.10 (m, 1H), 2.15 (s, 3H), 2.22 (s, 3H), 2.70-2.75 (m, 1H), 3.02-3.10 (m, |
| | 1H), 3.40-3.62 (m, 6H), 4.50-4.60 (m, 1H), 6.88-6.95 (m, 3H), 7.04 (s, 1H), 7.25-7.30 |
| | (m, 3H), 7.42 (s, 1H), 7.67 (s, 1H), 7.95 (s, 1H), 8.40 (s, 1H) |
| 12 | LC/MS: m/z 518.25 (M + H)⁺ (ES+), 97.86% |
| | ¹H NMR (400 MHZ, CD₃OD): δ 1.92-2.02 (m, 1H), 2.35-2.40 (m, 1H), 2.42 (s, 3H), |
| | 2.60-2.67 (m, 1H), 2.69 (s, 3H), 2.88-3.18 (m, 3H), 3.80-3.85 (m, 1H), 3.90-3.99 (m, |
| | 2H), 6.92-6.98 (m, 2H), 7.14-7.23 (m, 2H), 7.32-7.40 (m, 5H), 7.45-7.53 (m, 2H), 7.57- |
| | 7.61 (m, 1H), 7.65 (s, 1H ), 9.17 (s, 1H) |
| 13 | LC/MS: m/z 466.30 (M + H)⁺ (ES+), 96.04% |
| | ¹H NMR (400 MHZ, CD₃OD): δ 2.25-2.35 (m, 1H), 2.42 (s, 3H), 2.60-2.70 (m, 1H), |
| | 2.77 (s, 3H), 3.30-3.40 (m, 1H), 3.60-3.80 (m, 3H), 4.00-4.15 (m, 1H), 4.50-4.60 (m, |
| | 2H), 7.38-7.42 (m, 1H), 7.48-7.52 (m, 2H), 7.64 (t, J = 7.7 Hz, 1H), 7.71 (s, 1H), 7.73 |
| | (s, 1H), 7.79 (s, 1H), 7.88-7.92 (m, 2H), 7.97 (d, J = 8.0 Hz , 1H), 8.07 (s, 1H), 8.24 |
| | (m, 1H), 8.30 (s, 1H ), 9.24 (s, 1H). 14.55% TFA content. |
| 14 | LC/MS: m/z 467.34 (M + H)⁺ (ES+), 96.87% |
| | ¹H NMR (400 MHZ, CD₃OD): δ 2.03-2.13 (m, 1H), 2.47 (s, 3H), 2.50-2.59 (m, 1H), |
| | 2.74 (s, 3H), 2.88-2.93 (m, 1H), 3.16-3.26 (m, 1H), 3.36-3.70 (m, 2H), 3.89-4.00 (m, |
| | 1H), 4.22 (m, 2H), 7.50-7.60 (m, 4H), 7.80 - 7.85 (m, 3H), 7.92-7.97 (m, 1H), 8.12 (s, |
| | 1H), 8.29 (s, 1H), 8.51 (s, 1H), 8.77 (d, J = 5.1 Hz, 1H ), 9.34 (s, 1H) |
| 19 | LC/MS: m/z 483.34 (M + H)⁺ (ES+), 97.87% |
| | ¹H NMR (400 MHZ; DMSO-d₆ with D₂O): 0 1.40-1.50 (m, 1H), 1.53-1.63 (m, 1H), 1.80- |
| | 1.95 (m, 2H), 2.31 (s, 3H), 2.99-3.10 (m, 4H), 3.20-3.30 (m, 1H), 3.90-4.02 (m, 2H), |
| | 7.20-7.30 (m, 3H), 7.33-7.40 (m, 1H), 7.45-7.57 (m, 4H), 7.77 (s, 1H), 8.03 (s, 1H), |
| | 8.18 (s, 1H), 8.63 (d, J = 5.6 Hz, 1H), 9.18 (s, 1H) |
| 22 | LC/MS: m/z 481.21 (M + H)⁺ (ES+), 95.01% |
| | ¹H NMR (400 MHz; CD₃OD): δ 1.35-1.42 (m, 1H), 1.60-1.69 (m, 1H), 1.75-1.90 (m, |
| | 2H), 2.10-2.20 (m, 1H), 2.33 (s, 3H), 2.30-2.40 (m, 1H), 2.60-2.70 (m, 1H), 2.70-2.80 |
| | (m, 1H), 2.99-3.10 (m, 1H), 3.59 (s, 2H), 3.97 (s, 2H), 7.28 (d, J = 8.4 Hz, 2H), 7.40- |
| | 7.60 (m, 5H), 7.67 (d, J = 7.2 Hz, 2H), 7.75 (s, 1H), 7.98 (s, 1H), 8.08 (d, J = 2.4 Hz, |
| | 1H), 8.76 (d, J = 1.5 Hz, 1H) |
| 25 | LC/MS: m/z 504.37 (M + H)⁺ (ES+), 95.26% |
| | ¹H NMR (400 MHZ; DMSO-d₆): δ 0.90-1.00 (m, 1H), 1.40-1.50 (m, 1H), 1.51-1.62 (m, |
| | 3H), 1.65-1.75 (m, 2H), 1.90-2.00 (m, 3H), 2.15 (s, 3H), 2.39 (s, 3H), 2.60-2.75 (m, |
| | 2H), 3.25-3. 50 (m, 5H), 3.80-3.88 (m, 2H), 4.60-4.70 (m, 1H), 7.03 (s, 1H), 7.16 (d, J = |
| | 8.8 Hz, 1H), 7.28 (s, 1H), 7.32-7.38 (m, 2H), 7.63 (m, 1H), 7.96 (s, 1H), 8.18 (d, J = |
| | 2.8 Hz, 1H), 8.75 (s, 1H) |
| 26 | LC/MS: m/z 521.27 (M + H)⁺ (ES+), 97.93% |
| | ¹H NMR (400 MHZ; DMSO-d₆): δ 0.90-1.00 (m, 1H), 1.40-1.51 (m, 3H), 1.60-1.75 (m, |
| | 3H), 1.88-1.95 (m, 2H), 2.15 (s, 3H), 2.60-2.72 (m, 4H), 3.10-3.20 (m, 2H), 3.32-3.50 |
| | (m, 2H), 3.58-3. 62 (m, 1H), 3.65-3.73 (m, 2H), 4.52 (s, 2H), 7.02 (s, 1H), 7.17 (dd, J = |
| | 8.9, 8.8 Hz, 1H), 7.27 (s, 1H), 7.34 (s, 1H), 7.36-7.42 (m, 2H), 7.63 (s, 1H), 7.95 (d, J = |
| | 2.8 Hz, 1H), 8.69 (s, 1H) |
| 27 | LC/MS: m/z 521.29 (M + H)⁺ (ES+), 98.46% |
| | ¹H NMR (400 MHZ; DMSO-d₆): δ 0.90-1.00 (m, 1H), 1.40-1.51 (m, 3H), 1.53-1.75 (m, |
| | 3H), 1.85-1.95 (m, 3H), 2.15 (s, 3H), 2.60-2.72 (m, 3H), 3.12-3.20 (m, 2H), 3.37-3.47 |
| | (m, 2H), 3.58-3. 65 (m, 1H), 3.75-3.85 (m, 2H), 4.57 (s, 2H), 7.01 (s, 1H), 7.08-7.20 |
| | (m, 1H), 7.27 (s, 1H), 7.34 (s, 1H), 7.36-7.42 (m, 2H), 7.63 (s, 1H), 7.95 (s, 1H), 8.69 |
| | (s, 1H) |
| 28 | LC/MS: m/z 466.23 (M + H)⁺ (ES+), 98.55% |
| | ¹H NMR (400 MHZ; DMSO-d₆): δ 0.95-1.02 (m, 1H), 1.45-1.53 (m, 1H), 1.60-1.65 (m, |
| | 1H), 1.70-1.80 (m, 2H), 1.90-2.00 (m, 1H), 2.17 (s, 3H), 2.60-2.70 (m, 3H), 3.40-3.60 |
| | (m, 2H), 7.23 (s, 1H), 7.35 (s, 1H), 7.40-7.46 (m, 1H), 7.50-7.55 (m, 2H), 7.70 (s, 1H), |
| | 7.76 (d, J = 7.3 Hz, 1H), 7.85 (d, J = 8.3 Hz, 1H), 8.00 (s, 1H), 8.03 (s, 1H), 8.08 (d, J = |
| | 8.3 Hz, 1H), 10.48 (bs, 1H) |
| 29 | LC/MS (ESI): m/z = 467.0 [M + 1]⁺. RT = 5.482 min |
| | ¹H NMR (400 MHZ, DMSO-d₆) δ 10.84 (s, 1H), 8.82 (d, J = 5.2 Hz, 1H), 8.43 (d, J = |
| | 0.8 Hz, 1H), 8.08-8.00 (m, 3H), 7.97-7.90 (m, 3H), 7.61-7.52 (m, 3H), 7.40 (s, 1H), |
| | 7.27 (s, 1H), 3.54 (d, J = 13.6 Hz, 1H), 3.46 (d, J = 13.2 Hz, 1H), 2.75-2.67 (m, 3H), |
| | 2.18 (s, 3H), 1.97 (t, J = 9.6 Hz, 1H), 1.74-1.61 (m, 3H), 1.51-1.43 (m, 1H), 1.01-0.94 |
| | (m, 1H) |
| 30 | LC/MS: m/z 468.34 (M + H)⁺ (ES+), 95.53% |
| | ¹H NMR (400 MHZ; DMSO-d₆): δ 1.00-1.10 (m, 1H), 1.43-1.52 (m, 1H), 1.60-1.69 (m, |
| | 1H), 1.71-1.81 (m, 2H), 1.99-2.06 (m, 1H), 2.18 (s, 3H), 2.62-2.70 (m, 1H), 2.71-2.81 |
| | (m, 2H), 3.40-3.57 (s, 2H), 7.27 (s, 1H), 7.39 (s, 1H), 7.53-7.59 (m, 1H), 7.96 (s, 1H), |
| | 8.00-8.05 (m, 2H), 8.07 (s, 1H), 8.24 (t, J = 8.0 Hz, 1H), 8.36 (d, J = 3.6 Hz, 1H), 8.80 |
| | (d, J = 4 Hz, 1H), 8.84 (s, 1H), 8.87 (d, J = 5.2 Hz, 1H), 10.85 (bs, 1H) |
| 31 | LC/MS: m/z 473.27 (M + H)⁺ (ES+), 98.98% |
| | ¹H NMR (400 MHZ, DMSO-d₆): δ 0.89-1.02 (m, 1H), 1.40-1.52 (m, 1H), 1.60-1.69 (m, |
| | 1H), 1.70-1.81 (m, 2H), 1.92-2.00 (m, 1H), 2.17 (s, 3H), 2.62-2.72 (m, 3H), 3.41-3.60 |
| | (m, 2H), 7.24 (s, 1H), 7.36 (s, 1H), 7.63 (t, J = 7.6 Hz, 1H), 7.69 (s, 1H), 7.92-8.00 (m, |
| | 2H), 8.04 (s, 1H), 8.21 (dd, J = 7.6 Hz, 1H), 8.31 (d, J = 1.2 Hz, 1H), 8.58 (d, J = 1.6 |
| | Hz, 1H), 9.26 (d, J = 2.4 Hz, 1H), 10.56 (s, 1H) |
| 32 | LC/MS: m/z 516.99 (M + H)⁺ (ES+), 99.38% |
| | ¹H NMR (400 MHZ; DMSO-d₆): δ 0.98-1.03 (m, 1H), 1.44-1.49 (m, 1H), 1.60-1.70 (m, |
| | 5H), 1.93-1.98 (m, 3H), 1.82 (m, 4H), 2.16 (s, 3H), 2.63-2.70 (m, 3H), 3.14-3.17 (m, |
| | 1H), 3.61-3.70 (m, 2H), 4.36 (br, 1H), 7.18 (s, 1H), 7.32 (s, 1H), 7.39-7.41 (m, 2H), |
| | 7.45-7.46 (m, 3H), 7.72 (s, 1H), 7.88 (s, 1H), 8.00 (d, J = 1.3 Hz, 1H), 10.00 (bs, 1H) |
| 33 | LC/MS: m/z 491.35 (M + H)⁺ (ES+), 97.09% |
| | ¹H NMR (400 MHZ; DMSO-d₆): δ 0.95-1.02 (m, 1H), 1.40-1.50 (m, 1H), 1.60-1.80 (m, |
| | 4H), 1.83-2.00 (m, 4H), 2.15 (s, 3H), 2.60-2.70 (m, 6H), 3.35-3.50 (m, 2H), 3.61-3.70 |
| | (m, 2H), 6.95-7.07 (m, 4H), 7.14 (s, 1H), 7.30 (s, 1H), 7.43 (s, 1H), 7.83 (s, 1H), 7.98 |
| | (s, 1H), 10.17 (bs, 1H) |
| 34 | LC/MS: m/z 491.25 (M + H)⁺ (ES+), 97.14% |
| | ¹H NMR (400 MHZ; DMSO-d₆): δ 0.95-1.02 (m, 1H), 1.40-1.50 (m, 1H), 1.50-1.80 (m, |
| | 7H), 1.90-2.00 (m, 2H), 2.15 (s, 3H), 2.60-2.80 (m, 5H), 3.35-3.49 (m, 2H), 3.50-3.60 |
| | (m, 1H), 3.61-3.70 (m, 1H), 6.95-7.07 (m, 4H), 7.15 (s, 1H), 7.30 (s, 1H), 7.43 (s, 1H), |
| | 7.81 (s, 1H), 7.98 (s, 1H), 10.18 (bs, 1H) |
| 35 | LC/MS: m/z 467.20 (M + H)⁺ (ES+), 97.03% |
| | ¹H NMR (400 MHZ; DMSO-d₆): δ 1.32-1.40 (m, 1H), 1.52-1.64 (m, 1H), 1.80-1.90 (m, |
| | 2H), 2.05-2.25 (m, 2H), 2.27 (s, 3H), 2.60-2.70 (m, 1H), 2.90-2.95 (m, 1H), 3.60-3.80 |
| | (m, 3H), 7.32 (s, 1H), 7.35 (s, 1H), 7.38-7.43 (m, 1H), 7.49 (t, J = 7.5 Hz, 2H), 7.62 (t, |
| | J = 7.7 Hz, 1H), 7.70-7.75 (m, 3H), 7.86 (d, J = 7.7 Hz, 1H), 7.93 (s, 1H), 7.96 (d, J = |
| | 1.7 Hz, 1H), 8.05 (s, 1H), 8.21 (s, 1H) |
| 38 | LC/MS: m/z 466.38 (M + H)⁺ (ES+), 96.38% |
| | ¹H NMR (400 MHZ; DMSO-d₆ with D₂O): δ 1.40-1.52 (m, 1H), 1.60-1.70 (m, 1H), 1.80- |
| | 2.00 (m, 2H), 2.29 (s, 3H), 2.70-2.80 (m, 2H), 3.12-3.22 (m, 1H), 3.30-3.40 (m, 2H), |
| | 4.18 (s, 2H), 7.35-7.42 (m, 1H), 7.43-7.51 (m, 3H), 7.61-7.71 (m, 4H), 7.83-7.91 (m, |
| | 3H), 8.00 (s, 1H), 8.12 (s, 1H), 9.06 (s, 1H) |
| 39 | LC/MS: m/z 485.33 (M + H)⁺ (ES+), 98.37% |
| | ¹H NMR (400 MHZ; DMSO-d₆ in D₂O): δ 1.10-1.20 (m, 1H), 1.45-1.50 (m, 1H), 1.60- |
| | 1.69 (m, 1H), 1.70-1.80 (m, 1H), 1.90-2.00 (m, 1H), 2.05-2.13 (m, 1H), 2.17 (s, 3H), |
| | 2.55-2.65 (m, 1H), 2.70-2.78 (m, 1H), 2.88-2.93 (m, 1H), 3.48 - 3.52 (m, 2H), 7.29 (s, |
| | 1H), 7.35-7.43 (m, 3H), 7.89 (s, 1H), 7.91-8.01 (m, 4H), 8.07 (s, 1H), 8.38 (s, 1H), 8.78 |
| | (d, J = 5.2 Hz, 1H) |
| 40 | LC/MS: m/z 468.27 (M + H)⁺ (ES+), 95.56% |
| | ¹H NMR (400 MHZ; DMSO-d₆ in D₂O): δ 1.10-1.20 (m, 1H), 1.43-1.50 (m, 1H), 1.60- |
| | 1.69 (m, 1H), 1.70-1.80 (m, 2H), 1.95-2.05 (m, 1H), 2.18 (s, 3H), 2.63-2.70 (m, 1H), |
| | 2.70-2.80 (m, 1H), 3.48-3.52 (m, 2H), 7.32 (s, 1H), 7.41(s, 1H), 7.55-7.63 (m, 3H), |
| | 7.89 (s, 1H), 8.03 (s, 1H), 8.09 (s, 1H), 9.24 (d, J = 6.4 Hz, 1H), 9.52 (s, 1H), 10.72 |
| | (bs, 1H) |
| 42 | LC/MS: m/z 497.31 (M + H)⁺ (ES+), 99.06% |
| | ¹H NMR (400 MHZ, DMSO-d₆): δ 0.90-1.04 (m, 1H), 1.47-1.52 (m, 1H), 1.60-1.69 (m, |
| | 1H), 1.70-1.81 (m, 2H), 1.98-2.05 (m, 1H), 2.18 (s, 3H), 2.62-2.68 (m, 1H), 2.70-2.78 |
| | (m, 2H), 3.42-3.58 (m, 2H), 3.84 (s, 3H), 7.13 (d, J = 8.8 Hz, 1H), 7.26 (s, 1H), 7.39 (s, |
| | 1H), 7.88 (d, J = 8.8 Hz, 2H), 7.95 (s, 1H), 7.98 (dd, J = 1.6 Hz, 5.2 Hz, 1H), 8.03 (s, |
| | 1H), 8.07 (d, J = 1.2 Hz, 1H), 8.38 (d, J = 1.6 Hz, 1H), 8.74 (d, J = 5.2 Hz, 1H), 10.80 |
| | (s, 1H) |
| 43 | LC/MS: m/z 545.36 (M + H)⁺ (ES+), 98.65% |
| | ¹H NMR (400 MHz; DMSO-d₆): δ 1.00-1.10 (m, 1H), 1.40-1.52 (m, 1H), 1.70-1.80 (m, |
| | 2H), 1.97-2.05 (m, 1H), 2.18 (s, 3H), 2.60-2.69 (m, 1H), 2.70-2.80 (m, 2H), 3.35 (s, |
| | 3H), 3.40-3.61 (m, 3H), 7.27 (s, 1H), 7.39 (s, 1H), 7.86 (t, J = 7.6 Hz, 1H), 7.97 (s, |
| | 1H), 8.01-8.10 (m, 3H), 8.14 (d, J = 4.8 Hz, 1H), 8.29 (d, J = 7.6 Hz, 1H), 8.40 (s, 1H), |
| | 8.53 (s, 1H), 8.88 (d, J = 5.2 Hz, 1H), 10.86 (bs, 1H) |
| 44 | LC/MS: m/z 468.21 (M + H)⁺ (ES+), 95.54% |
| | ¹H NMR (400 MHZ; DMSO-d₆ with D₂O): δ 1.01-1.12 (m, 1H), 1.23 (s, 2H), 1.47-1.52 |
| | (m, 2H), 1.63-1.72 (m, 1H), 1.73-1.80 (m, 1H), 1.81-1.87 (m, 1H), 2.05-2.11 (m, 1H), |
| | 2.18 (s, 3H), 2.53-2.59 (m, 1H), 2.72-2.83 (m, 1H), 3.53-3.68 (m, 2H), 7.46 (s, 1H), |
| | 7.52-7.62 (m, 3H), 7.71 (s, 1H), 7.92 (d, J = 6.8 Hz, 2H), 8.07 (dd, J = 2.0 Hz, 5.2 Hz, |
| | 1H), 8.23 (s, 1H), 8.46 (s, 1H), 8.84 (d, J = 4.8 Hz, 1H), 10.52 (bs, 1H) |
| 45 | LC/MS: m/z 468.24 (M + H)⁺ (ES+), at 4.24 min, 96.96% |
| | ¹H NMR (400 MHZ; DMSO-d₆ at 353 K): δ 1.50-1.58 (m, 1H), 1.60-1.68 (m, 1H), 1.79- |
| | 1.91 (m, 2H), 2.25 (s, 3H), 2.53-2.61 (m, 1H), 2.70-2.78 (m, 1H), 2.86-2.91 (m, 1H), |
| | 3.79 (s, 2H), 7.53-7.62 (m, 3H), 7.66 (s, 1H), 7.78 (S, 1H), 7.88 (d, J = 7.2 Hz, 2H), |
| | 8.07 (d, J = 5.6 Hz, 1H), 8.46 (s, 1H), 8.50 (s, 1H), 8.82 (d, J = 4.8 Hz, 1H), 8.92 (s, |
| | 1H), 10.77 (s, 1H). |
| 46 | LC/MS: m/z 456.36 (M + H)⁺ (ES+), 99.44% |
| | ¹H NMR (400 MHZ, CD₃OD): δ 1.53-1.68 (m, 2H), 1.78-1.85 (m, 1H), 1.86-1.93 (m, |
| | 1H), 2.39 (s, 3H), 2.45-2.63 (m, 3H), 2.80-2.90 (m, 1H), 3.31-3.40 (m, 1H), 3.60-3.70 |
| | (m, 2H), 4.60 (s, 2H), 6.81 (d, J = 4.4 Hz, 2H), 6.88 (s, 1H), 7.45-7.55 (m, 3H), 7.59 (s, |
| | 1H), 7.64 (d, J = 5.2 Hz, 1H), 7.71 (d, J = 7.2 Hz, 1H), 7.79 (s, 1H), 8.57 (d, J = 5.2 |
| | Hz, 1H), 8.98 (s, 1H) |
| 47 | LC/MS: m/z 517.20 (M + H)⁺ (ES+), 97.69% |
| | ¹H NMR (400 MHZ; DMSO-d₆ with D₂O): δ 1.42-1.51 (m, 1H), 1.59-1.69 (m, 1H), 1.89- |
| | 1.99 (m, 2H), 2.34 (s, 3H), 2.60-2.71 (m, 2H), 3.01-3.23 (m, 2H), 3.31 (bs, 1H), 4.10 |
| | (bs, 1H), 6.97-7.25 (m, 1H), 7.53 (s, 1H), 7.74 (d, J = 4.8 Hz, 1H), 7.82 (s, 1H), 8.03- |
| | 8.10 (m, 3H), 8.12 (s, 1H), 8.25 (s, 1H), 8.42 (s, 1H), 8.85 (d, J = 5.2 Hz, 1H), 9.27 (s, |
| | 1H) |
| 48 | LC/MS: m/z 511.20 (M + H)⁺ (ES+), at 4.32 min, 95.26% |
| | ¹H NMR (400 MHZ; CD₃OD): δ 1.50 (d, J = 6.4 Hz, 3H), 1.60-1.75 (m, 2H), 1.81-1.95 |
| | (m, 2H), 2.38 (s, 3H), 2.50-2.60 (m, 3H), 2.75-2.82 (m, 1H), 3.38-3. 42 (m, 1H), 3.68- |
| | 3.75 (m, 2H), 4.95 (q, J = 6.4 Hz, 2H), 7.43 (s, 1H), 7.51-7.55 (m, 2H), 7.60 (s, 1H), |
| | 7.70-7.75 (m, 1H), 7.84 (s, 1H), 7.85-7.90 (m, 0.19H), 7.93-7.95 (m, 1.12H), 7.97 (s, |
| | 0.89H), 8.09 (m, 0.15H), 8.14 (br s, 0.85H), 8.40-8.41 (m, 0.15H), 8.52 (s, 1H), 8.58- |
| | 8.60 (m, 0.17H), 8.77 (d, J = 4.8 Hz, 2H) |
| 49 | LC/MS: m/z 471.40 (M + H)⁺ (ES+), 99.29% |
| | ¹H NMR (400 MHZ; DMSO-d₆ in D₂O): δ 1.40-1.52 (m, 1H), 1.60-1.68 (m, 1H), 1.80- |
| | 2.00 (m, 2H), 2.34 (s, 3H), 2.55-2.60 (m, 2H), 3.00-3.20 (m, 2H), 3.25-3.38 (m, 1H), |
| | 4.00-4.12 (m, 2H), 7.15-7.55 (m, 3H), 7.60-7.69 (m, 2H), 7.76 (d, J = 8 Hz, 1H), 7.82 |
| | (s, 1H), 8.02-8.09 (m, 1H), 8.13 (s, 1H), 8.25 (s, 1H), 8.41 (s, 1H), 8.84 (d, J = 5.2 Hz, |
| | 1H), 9.26 (s, 1H) |
| 50 | LC/MS: m/z 524.21 (M + H)⁺ (ES+), 96.16% |
| | ¹H NMR (400 MHZ; DMSO-d₆): δ 1.00-1.10 (m, 1H), 1.45-1.52 (m, 1H), 1.60-1.68 (m, |
| | 1H), 1.70-1.80 (m, 2H), 1.97-2.08 (m, 1H), 2.18 (s, 3H), 2.60-2.69 (m, 1H), 2.70-2.80 |
| | (m, 2H), 3.45-3.60 (m, 2H), 7.27 (s, 1H), 7.39 (s, 1H), 7.90-8.20 (m, 4H), 8.17 (d, J = 2 |
| | Hz, 1H), 8.39 (d, J = 8.4 Hz, 1H), 8.53 (s, 1H), 8.62 (s, 1H), 8.85 (d, J = 5.2 Hz, 1H), |
| | 9.51 (bs, 1H), 10.84 (bs, 1H) |
| 51 | LC/MS: m/z 507.39 (M + H)⁺ (ES+), 95.26% |
| | ¹H NMR (400 MHZ; DMSO-d₆): δ 1.10-1.20 (m, 1H), 1.45-1.53 (m, 1H), 1.60-1.70 (m, |
| | 1H), 1.72-1.80 (m, 2H), 2.00-2.07 (m, 1H), 2.18 (s, 3H), 2.60-2.70 (m, 1H), 2.72-2.80 |
| | (m, 2H), 3.46-3.60 (m, 2H), 7.29 (s, 1H), 7.36-7.49 (m, 3H), 7.84 (d, J = 7.6 Hz, 1H), |
| | 7.89 (d, J = 8.0 Hz, 1H), 7.97 (s, 1H), 8.05-8.09 (m, 2H), 8.12-8.16 (m, 1H), 8.47 (s, |
| | 1H), 8.91 (s, 1H), 8.94 (d, J = 5.6 Hz, 1H), 10.91 (bs, 1H). |
| 52 | LC/MS: m/z 471.40 (M + H)⁺ (ES+), at 4.08 min, 99.29% |
| | ¹H NMR (400 MHZ; DMSO-d₆): δ 0.98-1.15 (m, 1H), 1.45-1.52 (m, 1H), 1.60-1.68 (m, |
| | 1H), 1.70-1.80 (m, 2H), 1.97-2.05 (m, 1H), 2.18 (s, 3H), 2.19 (s, 3H), 2.62-2.75 (m, |
| | 3H), 3.45-3.59 (m, 2H), 7.27 (s, 1H), 7.37 (s, 1H), 7.95 (s, 1H), 7.98-8.01 (m, 1H), 8.02 |
| | (s, 1H), 8.09 (s, 1H), 8.36 (d, J = 2H, 1H), 8.57 (s, 1H), 8.78 (d, J = 5.2 Hz, 1H), 10.84 |
| | (bs, 1H) |
| 53 | LC/MS (ESI): m/z = 523.3 [M + 1]⁺ RT = 6.44 min |
| | ¹H NMR (400 MHZ, DMSO-d₆) δ 10.84 (s, 1H), 8.81 (d, J = 5.2 Hz, 1H), 8.43 (d, J = |
| | 1.6 Hz, 1H), 8.09-8.03 (m, 3H), 7.97 (s, 1H), 7.92-7.90 (m, 2H), 7.63-7.54 (m, 4H), |
| | 7.41 (s, 1H), 7.30 (s, 1H), 3.78-3.71 (m, 1H), 3.60-3.49 (m, 2H). 2.79-2.67 (m, 2H), |
| | 2.18 (s, 3H), 2.07-2.01 (m, 3H), 1.89-1.83 (m, 1H), 1.72-1.65 (m, 2H), 1.56-1.46 (m, |
| | 1H), 1.25-1.22 (m, 1H), 0.93 (t, J = 7.2 Hz, 3H) |
| 54 | LC/MS (ESI): m/z = 421.3 [M + 1]⁺ RT = 4.24 min |
| | ¹H NMR (400 MHZ, DMSO-d₆) δ 10.58 (s, 1H), 8.73 (dd, J = 4.4, 1.6 Hz, 1H), 8.50 (d, |
| | J = 2.8 Hz, 1H), 8.04 (d, J = 1.2 Hz, 1H), 7.95 (d, J = 2 Hz, 1H), 7.87 (dd, J = 2.8, 2.0 |
| | Hz, 1H), 7.65 (s, 1H), 7.36 (s, 1H), 7.26 (s, 1H), 3.93 (s, 3H), 3.53 (d, J = 13.6 Hz, |
| | 1H). 3.43 (d, J = 13.2 Hz, 1H), 2.74-2.65 (m, 3H), 2.18 (s, 3H), 1.98-1.93 (m, 1H), |
| | 1.74-1.61 (m, 3H), 1.48-1.45 (m, 1H), 0.99-0.96 (m, 1H) |
| 55 | LC/MS (ESI): m/z = 483.1 [M + 1]⁺ RT = 4.95 min |
| | ¹H NMR (400 MHZ, DMSO-d₆) δ 10.79 (s, 1H), 8.72 (d, J = 5.2 Hz, 1H), 8.35 (d, J = |
| | 1.2 Hz, 1H), 8.08-8.03 (m, 2H), 7.96-7.92 (m, 2H), 7.77 (d, J = 8.8 Hz, 2H), 7.40 (s, |
| | 1H), 7.26 (s, 1H), 6.94 (d, J = 8.8 Hz, 2H), 3.54 (d, J = 13.6 Hz, 1H). 3.46 (d, J = 13.2 |
| | Hz, 1H), 2.75-2.67 (m, 3H), 2.18 (s, 3H), 2.01-1.96 (m, 1H), 1.73-1.62 (m, 3H), 1.52- |
| | 1.43 (m, 1H), 1.01-0.93 (m, 1H) |
| 56 | LC/MS (ESI): m/z = 473.2 [M + 1]⁺ RT = 5.83 min |
| | ¹H NMR (400 MHZ, DMSO-d₆) δ 8.55 (s, 1H), 8.05 (d, J = 0.8 Hz, 1H), 7.98 (s, 1H), |
| | 7.85-7.77 (m, 3H), 7.53-7.44 (m, 3H), 7.36 (s, 1H), 7.27 (s, 1H), 3.53 (d, J = 13.6 Hz, |
| | 1H). 3.46 (d, J = 13.2 Hz, 1H), 2.74-2.63 (m, 3H), 2.18 (s, 3H), 2.0-1.96 (m, 1H), 1.72- |
| | 1.61 (m, 3H), 1.52-1.46 (m, 1H), 1.01-0.98 (m, 1H) |
| 57 | LC/MS (ESI): m/z = 457.3 [M + 1]⁺ RT = 5.62 min |
| | ¹H NMR (400 MHZ, DMSO-d₆) δ 8.05-7.93 (m, 4H), 7.98 (s, 1H), 7.75 (s, 1H), 7.62- |
| | 7.52 (m, 4H), 7.36 (s, 1H), 7.28 (s, 1H), 3.53 (d, J = 13.6 Hz, 1H). 3.46 (d, J = 14 Hz, |
| | 1H), 2.74-2.67 (m, 3H), 2.18 (s, 3H), 2.0-1.95 (m, 1H), 1.71-1.61 (m, 3H), 1.49-1.46 |
| | (m, 1H), 0.99-0.97 (m, 1H) |
| 58 | LC/MS (ESI): m/z = 456.3 [M + 1]⁺ RT = 5.05 min |
| | ¹H NMR (400 MHZ, DMSO-d₆) δ 10.12 (s, 1H), 8.5 (d, J = 1.2 Hz, 1H), 8.46 (d, J = 1.2 |
| | Hz, 1H), 8.04 (s, 1H), 7.98 (s, 1H), 7.87 (s, 1H), 7.80 (d, J = 7.6 Hz, 2H), 7.57 (dd, J = |
| | 8.4, 7.6 Hz, 2H), 7.44 (t, J = 7.6 Hz, 1H), 7.36 (s, 1H), 7.20 (s, 1H), 3.52 (d, J = 13.6 |
| | Hz, 1H). 3.44 (d, J = 13.2 Hz, 1H), 2.74-2.61 (m, 3H), 2.18 (s, 3H), 2.0-1.95 (m, 1H), |
| | 1.72-1.61 (m, 3H), 1.51-1.46 (m, 1H), 1.01-0.98 (m, 1H) |
| 59 | LC/MS (ESI): m/z = 405.1 [M + 1]⁺ RT = 4.71 min |
| | ¹H NMR (400 MHZ, DMSO-d₆) δ 10.70 (s, 1H), 8.59 (s, 1H), 8.08-8.06 (m, 2H), 8.01 |
| | (s, 1H), 7.92-7.88 (m, 2H), 7.38 (s, 1H), 7.24 (s, 1H), 3.52 (d, J = 13.2 Hz, 1H), 3.44 |
| | (d, J= 13.2 Hz, 1H), 2.74-2.66 (m, 3H), 2.43 (s, 3H), 2.18 (s, 3H), 2.0-1.95 (m, 1H), |
| | 1.73-1.61 (m, 3H), 1.51-1.42 (m, 1H), 1.02-0.97 (m, 1H) |
| 60 | LC/MS: m/z 586.46 (M + H)⁺ (ES+), 98.56% |
| | ¹H NMR (400 MHz; DMSO-d₆): δ 0.90-1.00 (m, 1H), 1.40-1.51 (m, 3H), 1.53-1.75 (m, |
| | 3H), 1.85-1.95 (m, 3H), 2.15 (s, 3H), 2.60-2.72 (m, 3H), 3.12-3.20 (m, 2H), 3.37-3.47 |
| | (m, 2H), 3.58-3.65 (m, 1H), 3.75-3.85 (m, 2H), 4.57 (s, 2H), 7.18-7.21 (m, 1H), 7.25- |
| | 7.32 (m, 2H), 7.39 (s, 1H), 7.50-7.60 (m, 3H), 7.69 (d, J = 6 Hz, 1H ), 7.88 (d, J = 6.8 |
| | Hz, 2H), 7.96 (s, 1H), 8.00-8.06 (m, 3H), 8.07 (s, 1H), 8.42 (s, 2H), 8.79 (d, J = 5.2 Hz, |
| | 1H), 10.89 (bs, 1H) |
| 61 | LC/MS: m/z 559.46 (M + H)⁺ (ES+), 95.45% |
| | ¹H NMR (400 MHZ; DMSO-d₆): δ 1.10-1.20 (m, 3H), 1.30-1.49 (m, 2H), 1.60-1.72 (m, |
| | 1H), 1.85-2.00 (m, 2H), 2.26 (s, 3H), 2.60-2.72 (m, 1H), 3.00-3.10 (m, 2H), 3.57-3.62 |
| | (m, 2H), 3.92-4.02 (m, 1H), 4.34-4.52 (s, 2H), 7.51-7.70 (m, 5H ), 7.88 (d, J = 6.8 Hz, |
| | 2H), 8.06 (s, 1H), 8.20-8.40 (m, 1H), 8.42 (s, 1H), 8.83 (d, J = 5.2 Hz, 1H), 9.94 (bs, |
| | 1H), 11.08 (bs, 1H) |
| 62 | LC/MS: m/z 580.30 (M + H)⁺ (ES+), 96.32% |
| | ¹H NMR (400 MHZ; DMSO-d₆): δ 1.10-1.20 (m, 3H), 1.30-1.49 (m, 2H), 1.60-1.72 (m, |
| | 1H), 1.85-2.00 (m, 2H), 2.26 (s, 3H), 2.60-2.72 (m, 1H), 3.00-3.10 (m, 2H), 3.57-3.62 |
| | (m, 2H), 3.92-4.02 (m, 1H), 4.34-4.52 (s, 2H), 7.91 (d, J = 8.4 Hz, 1H ), 7.97(s, 1H), |
| | 8.00-8.06 (m, 2H), 8.07 (s, 1H), 8.42 (s, 1H), 8.80 (d, J = 5.2 Hz, 1H), 10.82 (bs, 1H) |
| 63 | LC/MS (ESI): m/z = 430.0 [M + 1]⁺ RT = 6.81 min |
| | ¹H NMR (400 MHZ, DMSO-d₆) δ 10.11 (s, 1H), 8.57-8.54 (m, 2H), 8.17 (d, J = 9.2 Hz, |
| | 1H), 8.04-8.01 (m, 2H), 7.90 (s, 1H), 7.37 (s, 1H), 7.25-7.17 (m, 2H), 6.97-6.94 (m, |
| | 1H), 3.52 (d, J = 13.2 Hz, 1H), 3.44 (d, J = 13.2 Hz, 1H), 2.74-2.66 (m, 3H), 2.43 (s, |
| | 3H), 2.18 (s, 3H), 2.0-1.95 (m, 1H), 1.73-1.61 (m, 3H), 1.51-1.42 (m, 1H), 1.02-0.97 |
| | (m, 1H) |
| 64 | LC/MS (ESI): m/z = 541.1 [M + 1]⁺. RT = 5.67 min |
| | ¹H NMR (400 MHZ, DMSO-d₆) δ 10.81 (s, 1H), 8.75 (d, J = 5.2 Hz, 1H), 8.39 (d, J = |
| | 1.6 Hz, 1H), 8.05-7.96 (m, 4H), 7.88 (d, J = 8.8 Hz, 2H), 7.39 (s, 1H), 7.26 (s, 1H), |
| | 7.14 (d, J = 8.8 Hz, 2H), 4.19 (t, J = 4.6 Hz, 2H), 3.70 (t, J = 4.4 Hz, 2H), 3.54 (d, J = |
| | 13.2 Hz, 1H), 3.46 (d, J = 13.2 Hz, 1H), 3.34 (s, 3H), 2.75-2.64 (m, 3H), 2.18 (s, 3H), |
| | 2.01-1.97 (m, 1H), 1.85-1.44 (m, 4H), 1.02-0.92 (m, 1H) |
| 65 | LC/MS (ESI): m/z = 481.1 [M + 1]⁺. RT = 5.99 min |
| | ¹H NMR (400 MHZ, DMSO-d₆) δ 10.78 (s, 1H), 8.70 (s, 1H), 8.07 (s, 1H), 8.02 (s, 1H), |
| | 7.97-7.95 (m, 2H), 7.57-7.48 (m, 5H), 7.39 (s, 1H), 7.25 (s, 1H), 3.53 (d, J = 13.6 Hz, |
| | 1H), 3.45 (d, J = 13.2 Hz, 1H), 2.74-2.64 (m, 3H), 2.44 (s, 3H), 2.18 (s, 3H), 2.01-1.91 |
| | (m, 1H), 1.73-1.42 (m, 4H), 1.01-0.93 (m, 1H) |
| 66 | LC/MS (ESI): m/z = 447.2 [M + 1]⁺. RT = 5.70 min |
| | ¹H NMR (400 MHZ, DMSO-d₆) δ 10.76 (s, 1H), 8.66 (d, J = 4.8 Hz, 1H), 8.17-7.94 (m, |
| | 4H), 7.72 (d, J = 3.6 Hz, 1H), 7.53-7.39 (m, 1H), 7.26 (s, 1H), 3.52-3.48 (m, 2H), 2.84- |
| | 2.67 (m, 3H), 2.18 (s, 3H), 1.97-1.64 (m, 4H), 1.50-1.47 (m, 1H), 1.35 (s, 9H), 1.15- |
| | 0.93 (m, 1H) |
| 67 | LC/MS (ESI): m/z = 478.1 [M + 1]⁺. RT = 5.76 min |
| | ¹H NMR (400 MHZ, DMSO-d₆) δ 10.79 (s, 1H), 8.81 (d, J = 5.2 Hz, 1H), 8.76 (d, J = 2 |
| | Hz, 1H), 8.43 (d, J = 1.2 Hz, 1H), 8.14 (s, 1H), 8.03-8.01 (m, 2H), 7.96 (dd, J₁ = 8.0 |
| | Hz, J₂ = 2.0 Hz, 1H), 7.92-7.90 (m, 2H), 7.61-7.52 (m, 3H), 7.38-7.36 (m, 2H), 3.57 (d, |
| | J = 13.2 Hz, 1H), 3.48 (d, J = 13.2 Hz, 1H), 2.78-2.67 (m, 3H), 2.53 (s, 3H), 1.97 (t, J = |
| | 10.0 Hz, 1H), 1.74-1.61 (m, 3H), 1.52-1.46 (m, 1H), 1.01-0.96 (m, 1H) |
| 68 | LC/MS (ESI): m/z = 507.1 [M + 1]⁺. RT = 5.71 min |
| | ¹H NMR (400 MHZ, DMSO-d₆) δ 10.89 (s, 1H), 8.87 (d, J = 5.6 Hz, 1H), 8.64-8.61 (m, |
| | 2H), 8.24-8.17 (m, 2H), 8.08-8.06 (m, 2H), 8.01-7.96 (m, 2H), 7.67 (t, J = 7.4 Hz, 1H), |
| | 7.43-7.40 (m, 2H), 7.28 (s, 1H), 3.55 (d, J = 13.6 Hz, 1H), 3.47 (d, J = 13.6 Hz, 1H), |
| | 2.76-2.67 (m, 3H), 2.19 (s, 3H), 2.0-1.95 (m, 1H), 1.74-1.42 (m, 4H), 1.02-0.94 (m, 1H) |
| 69 | LC/MS (ESI): m/z = 485.1 [M + 1]⁺. RT = 6.05 min |
| | ¹H NMR (400 MHZ, DMSO-d₆) δ 10.85 (s, 1H), 8.82 (d, J = 5.2 Hz, 1H), 8.43 (d, J = |
| | 1.2 Hz, 1H), 8.08-8.03 (m, 3H), 7.98 (s, 1H), 7.91 (d, J = 7.2 Hz, 2H), 7.61-7.54 (m, |
| | 3H), 7.40 (s, 1H), 7.29 (s, 1H), 4.29-4.12 (m, 1H), 3.58 (d, J = 13.6 Hz, 1H), 3.51 (d, J = |
| | 13.6 Hz, 1H), 2.87-2.74 (m, 3H), 2.18 (s, 3H), 2.04-1.91 (m, 2H), 1.77-1.66 (m, 2H) |
| 70 | LC/MS: m/z 527.29 (M + H)⁺ (ES+), 97.80% |
| | ¹H NMR (400 MHz; DMSO-d₆): δ 1.00-1.10 (m, 1H), 1.43-1.52 (m, 1H), 1.60-1.70 (m, |
| | 1H), 1.70-1.80 (m, 2H), 2.00-2.10 (m, 1H), 2.18 (s, 3H), 2.60-2.69 (m, 1H), 2.70-2.82 |
| | (m, 2H), 3.45-3.60 (m, 2H), 3.84 (s, 3H), 3.90 (s, 3H), 7.15 (d, J = 8.4 Hz, 1H), 7.27 (s, |
| | 1H), 7.38 (s, 1H), 7.42-7.50 (m, 2H), 7.79 (s, 1H), 8.00-8.09 (m, 2H), 8.07 (s, 1H), 8.40 |
| | (s, 1H), 8.74 (d, J = 5.2 Hz, 1H), 10.80 (bs, 1H) |
| 71 | LC/MS: m/z 511.21 (M + H)⁺ (ES+), 96.47% |
| | ¹H NMR (400 MHZ; DMSO-d₆ in D₂O): δ 1.40-1.49 (m, 1H), 1.50-1.62 (m, 1H), 1.80- |
| | 1.92 (m, 2H), 2.25 (s, 3H), 2.50-2.60 (m, 1H), 2.85-3.01 (m, 2H), 3.21-3.32 (m, 1H). |
| | 3.85-4.00 (m, 2H), 6.08 (s, 2H), 7.09 (d, J = 8 Hz, 1H), 7.40 (d, J = 8 Hz, 1H), 7.42- |
| | 7.48 (m, 2H), 7.76 (s, 1H), 7.93-7.95 (m, 1H), 8.06 (s, 1H), 8.20 (s, 1H), 8.32 (s, 1H), |
| | 8.75 (d, J = 5.2 Hz, 1H), 9.10 (s, 1H). |
| 72 | LC/MS (ESI): m/z = 473.2 [M + 1]⁺. RT = 5.50 min |
| | ¹H NMR (400 MHZ, DMSO-d₆) δ 10.72 (s, 1H), 8.63 (d, J = 4.8 Hz, 1H), 8.06-8.00 (m, |
| | 3H), 7.92 (s, 1H), 7.57 (d, J = 4.8 Hz, 1H), 7.38 (s, 1H), 7.25 (s, 1H), 3.53 (d, J = 13.2 |
| | Hz, 1H), 3.45 (d, J = 13.6 Hz, 1H), 2.74-2.64 (m, 4H), 2.18 (s, 3H), 2.0-1.95 (m, 1H), |
| | 1.86-1.80 (m, 4H), 1.74-1.60 (m, 4H), 1.51-1.38 (m, 5H), 1.32-1.23 (m, 1H), 1.02-0.95 |
| | (m, 1H) |
| 73 | LC/MS (ESI): m/z = 503.1 [M + 1]⁺. RT = 5.59 min |
| | ¹H NMR (400 MHZ, DMSO-d₆) δ 8.43 (s, 1H), 8.04 (d, J = 1.2 Hz, 1H), 7.97 (s, 1H), |
| | 7.85 (s, 1H), 7.77 (d, J = 8.8 Hz, 2H), 7.36 (s, 1H), 7.26 (s, 1H), 7.06 (d, J = 8.8 Hz, |
| | 2H), 3.82 (s, 3H), 3.52 (d, J = 14.0 Hz, 1H), 3.45 (d, J = 13.6 Hz, 1H), 2.74-2.65 (m, |
| | 3H), 2.18 (s, 3H), 1.97 (t, J = 9.2 Hz 1H), 1.73-1.61 (m, 3H), 1.51-1.43 (m, 1H), 1.01- |
| | 0.94 (m, 1H) |
| 74 | LC/MS (ESI): m/z = 457.1 [M + 1]⁺. RT = 5.13 min |
| | ¹H NMR (400 MHZ, DMSO-d₆) δ 8.05 (s, 2H), 7.95 (t, J = 2 Hz, 1H), 7.93-7.88 (m, 2H), |
| | 7.82-7.77 (s, 1H), 7.58-7.54 (m, 2H), 7.49-7.46 (m, 1H), 7.36 (s, 1H), 7.28 (s, 1H), |
| | 3.54 (d, J = 13.6 Hz, 1H), 3.46 (d, J = 13.6 Hz, 1H), 2.74-2.64 (m, 3H), 2.18 (s, 3H), |
| | 1.99-1.94 (m, 1H), 1.73-1.61 (m, 3H), 1.51-1.45 (m, 1H), 1.01-0.94 (m, 1H) |
| 75 | LC/MS (ESI): m/z = 467.1 [M + 1]⁺. RT = 5.49 min |
| | ¹H NMR (400 MHz, DMSO-d₆) δ 10.84 (s, 1H), 8.81 (d, J = 5.2 Hz, 1H), 8.43 (d, J = |
| | 1.2 Hz, 1H), 8.08-8.02 (m, 3H), 7.97-7.90 (m, 3H), 7.61-7.52 (m, 3H), 7.39 (s, 1H), |
| | 7.27 (s, 1H), 3.54 (d, J = 13.2 Hz, 1H), 3.46 (d, J = 13.6 Hz, 1H), 2.84-2.66 (m, 3H), |
| | 2.18 (s, 3H), 2.0-1.95 (m, 1H), 1.87-1.62 (m, 3H), 1.52-1.46 (m, 1H), 1.17-0.97 (m, 1H) |
| 76 | LC/MS: m/z 430.22 (M + H)⁺ (ES+), 98.84% |
| | ¹H NMR (400 MHZ, DMSO-d₆): δ 0.98-1.03 (m, 1H), 1.43-1.56 (m, 1H), 1.60-1.68 (m, |
| | 1H), 1.70-1.78 (m, 2H), 1.98-2.03 (m, 1H), 2.18 (s, 3H), 2.62-2.68 (m, 1H), 2.70-2.79 |
| | (m, 2H), 3.48-3.58 (m, 2H), 7.14 (s, 1H), 7.38-7.43 (m, 2H), 7.63 (t, J = 8.4 Hz, 1H), |
| | 7.79 (s, 1H), 7.90 (s, 1H), 7.94 (d, J = 8 Hz, 1H), 8.06 (s, 1H), 8.34 (d, J = 8.4 Hz, 1H), |
| | 8.55 (s, 1H), 10.55 (bs, 1H) |
| 77 | LC/MS (ESI): m/z = 541.2 [M + 1]⁺. RT = 5.53 min |
| | ¹H NMR (400 MHZ, DMSO-d₆) δ 10.83 (s, 1H), 8.80 (d, J = 5.2 Hz, 1H), 8.42 (d, J = |
| | 1.2 Hz, 1H), 8.08-8.03 (m, 3H), 7.97 (s, 1H), 7.51-7.39 (m, 4H), 7.26 (s, 1H), 7.11 (d, J = |
| | 7.6 Hz, 1H), 4.23 (t, J = 4.4 Hz, 2H), 3.71 (t, J = 4.6 Hz, 2H), 3.54 (d, J = 13.2 Hz, |
| | 1H), 3.46 (d, J = 13.6 Hz, 1H), 3.34 (s, 3H), 2.75-2.65 (m, 3H), 2.18 (s, 3H), 1.98 (t, J = |
| | 9.4 Hz, 1H), 1.74-1.62 (m, 3H), 1.52-1.43 (m, 1H), 1.03-0.98 (m, 1H) |
| 78 | LC/MS: m/z 580.33 (M + H)⁺ (ES+), 98.36% |
| | ¹H NMR (400 MHZ, DMSO-d₆): δ 1.32-1.40 (m, 1H), 1.45-1.70 (m, 4H), 2.05-2.34 (m, |
| | 2H), 2.18 (s, 3H), 2.20-2.25 (m, 1H), 2.78 (t, J = 6.8 Hz, 2H), 2.95-3.02 (m, 2H), 3.50- |
| | 3.60 (m, 4H), 3.76-3.86 (m, 1H), 4.15-4.21 (m, 1H), 5.26 (d, J = 6.4 Hz, 1H), 7.28 (s, |
| | 1H), 7.39 (s, 1H), 7.45 (d, J = 8.4 Hz 1H), 7.50-7.60 (m, 3H), 7.89 (d, J = 6.8 Hz, 2H), |
| | 7.96 (s, 1H), 8.00-8.07 (m, 2H ), 8.07 (s, 1H), 8.42 (s, 1H), 8.81 (d, J = 4.8 Hz, 1H), |
| | 10.82 (bs, 1H) |
| 79 | LC/MS: m/z 483.25 (M + H)⁺ (ES+), 96.00% |
| | ¹H NMR (400 MHZ, DMSO-d₆): δ 0.90-1.04 (m, 1H), 1.30-1.42 (m, 1H), 1.50-1.59 (m, |
| | 1H), 1.60-1.71 (m, 2H), 1.80-1.90 (m, 1H), 2.18 (s, 3H), 2.60-2.70 (m, 2H), 3.32-3.43 |
| | (m, 3H), 3.80 (s, 3H), 4.44 (d, J = 5.6 Hz, 2H), 6.45 (d, J = 6.0 Hz, 2H), 6.60 (s, 1H), |
| | 6.74 (s, 1H), 7.01 (d, J = 4.8 Hz, 1H), 7.24 (s, 1H), 7.52-7.58 (m, 1H), 7.68-7.70 (m, |
| | 3H), 7.92 (s, 1H), 8.52 (d, J = 5.2 Hz, 1H) |
| 80 | LC/MS (ESI): m/z = 479.1 [M + 1]⁺. RT = 5.99 min |
| | ¹H NMR (400 MHZ, DMSO-d₆) δ 10.84 (s, 1H), 8.82 (d, J = 5.2 Hz, 1H), 8.57 (s, 1H), |
| | 8.43 (d, J = 1.2 Hz, 1H), 8.09-8.07 (m, 2H), 8.03 (dd, J1 = 5.2 Hz, J2 = 2.0 Hz, 1H), |
| | 7.93-7.90 (m, 2H), 7.82 (s, 1H), 7.69 (d, J = 8.8 Hz, 1H), 7.62-7.53 (m, 3H), 3.60 (d, J = |
| | 13.6 Hz, 1H), 3.52 (d, J = 13.2 Hz, 1H), 2.79-2.68 (m, 6H), 2.01-1.95 (m, 1H), 1.78- |
| | 1.62 (m, 3H), 1.50-1.47 (m, 1H), 0.99-0.96 (m, 1H) |
| 81 | LC/MS (ESI): m/z = 638.2 [M + 1]⁺. RT = 6.78 min |
| | ¹H NMR (400 MHZ, DMSO-d₆) δ 10.84 (s, 1H), 10.72 (s, 1H), 8.81 (d, J = 5.2 Hz, 1H), |
| | 8.42 (d, J = 1.2 Hz, 1H), 8.09-7.89 (m, 6H), 7.71 (d, J = 8.8 Hz, 1H), 7.61-7.48 (m, |
| | 4H), 7.41 (s, 1H), 7.30-7.28 (m, 2H), 7.06-6.91 (m, 3H), 3.80-3.77 (m, 1H), 3.55-3.53 |
| | (m, 2H), 2.88 (d, J = 7.6 Hz, 2H), 2.78-2.67 (m, 2H), 2.43-2.38 (m, 2H), 2.17 (s, 3H), |
| | 2.01-1.83 (m, 1H), 1.71-1.49 (m, 4H), 1.23-1.13 (m, 1H) |
| 82 | LC/MS (ESI): m/z = 497.1 [M + 1]⁺. RT = 5.58 min |
| | ¹H NMR (400 MHZ, DMSO-d₆) δ 10.83 (s, 1H), 8.81 (d, J = 5.6 Hz, 1H), 8.41 (d, J = |
| | 1.2 Hz, 1H), 8.08-8.02 (m, 3H), 7.97 (s, 1H), 7.52-7.39 (m, 4H), 7.26 (s, 1H), 7.11 (dd, |
| | J = 6.4, 1.2 Hz, 1H), 3.88 (s, 3H), 3.54 (d, J = 13.6 Hz, 1H), 3.46 (d, J = 13.6 Hz, 1H), |
| | 2.75-2.65 (m, 3H), 2.18 (s, 3H), 2.00-1.93 (m, 1H), 1.74-1.42 (m, 4H), 1.04-0.94 (m, |
| | 1H) |
| 83 | LC/MS (ESI): m/z = 530.0 [M + 1]⁺. RT = 5.14 min |
| | ¹H NMR (400 MHZ, DMSO-d₆) δ 9.51 (s, 1H), 8.72 (s, 1H), 8.61 (d, J = 1.2 Hz, 1H), |
| | 8.32 (d, J = 8.4 Hz, 1H), 8.05 (s, 1H), 7.99 (s, 1H), 7.95 (dd, J = 8.4, 1.6 Hz, 1H), 7.87 |
| | (s, 1H), 7.37 (s, 1H), 7.28 (s, 1H), 3.54 (d, J = 13.6 Hz, 1H), 3.46 (d, J = 13.6 Hz, 1H), |
| | 2.74-2.64 (m, 3H), 2.18 (s, 3H), 2.01-1.96 (m, 1H), 1.73-1.46 (m, 4H), 1.01-0.98 (m, |
| | 1H) |
| 84 | LC/MS (ESI): m/z = 511.1 [M + 1]⁺. RT = 5.78 min |
| | ¹H NMR (400 MHZ, DMSO-d₆) δ 10.76 (s, 1H), 8.66(s, 1H), 8.07(d, J = 1.2 Hz, 1H), |
| | 8.02 (t, J = 1.6 Hz, 1H), 7.96-7.95(m, 2H), 7.49-7.46 (m, 2H), 7.39 (s, 1H), 7.25 (s, |
| | 1H), 7.12-7.09 (m, 2H), 3.84(s, 3H), 3.53 (dd, J = 13.6 Hz, 1H), 3.45 (dd, J = 13.6 Hz, |
| | 1H), 2.75-2.64 (m, 3H), 2.33 (s, 3H), 2.18 (s, 3H), 2.00-1.95 (m, 1H), 1.73-1.61 (m, |
| | 3H), 1.48-1.45 (m, 1H), 1.00-0.97 (m, 1H) |
| 85 | LC/MS: m/z 525.27 (M + H)⁺ (ES+), 95.19% |
| | ¹H NMR (400 MHZ; DMSO-d₆): δ 1.33-1.43 (m, 1H), 1.50-1.60 (m, 1H), 1.73-1.80 (m, |
| | 1H), 2.18 (s, 3H), 2.20-2.30 (m, 1H), 2.50-2.60 (m, 1H), 2.70-2.75 (m, 1H), 3.10-3.20 |
| | (m, 1H), 3.50-3.62 (m, 2H), 4.32 (s, 2H), 7.03 (d, J = 8.4 Hz, 1H), 7.32 (s, 1H), 7.38- |
| | 7.45 (m, 3H), 7.95-8.00 (m, 2H), 8.04 (s, 1H), 8.09 (s, 1H), 8.33 (s, 1H), 8.73 (d, J = |
| | 5.2 Hz, 1H), 10.78 (bs, 1H) |
| 86 | LC/MS: m/z 481.24 (M + H)⁺ (ES+), 95.38% |
| | ¹H NMR (400 MHZ, DMSO-d₆): δ 0.90-1.03 (m, 1H), 1.41-1.50 (m, 1H), 1.60-1.68 (m, |
| | 1H), 1.70-1.78 (m, 2H), 1.90-2.00 (m, 1H), 2.17 (s, 3H), 2.62-2.68 (m, 1H), 2.69-2.79 |
| | (m, 2H), 3.40-3.53 (m, 2H), 7.08 (s, 1H), 7.28-7.35 (m, 3H), 7.44 (t, J = 7.6 Hz, 2H), |
| | 7.52-7.69 (m, 7H), 8.01 (s, 1H), 8.86 (bs, 1H), 8.93 (bs, 1H) |
| 87 | LC/MS (ESI): m/z = 805.3 [M + 1]⁺. RT = 5.59 min |
| | ¹H NMR (400 MHZ, DMSO-d₆) δ 10.81 (s, 1H), 8.75 (d, J = 5.2 Hz, 1H), 8.39 (s, 1H), |
| | 8.07-8.02 (m, 2H), 7.99-7.93 (m, 2H), 7.88 (d, J = 8.4 Hz, 2H), 7.39 (s, 1H), 7.26 (s, |
| | 1H), 7.14 (d, J = 8.4 Hz, 2H), 4.19 (s, 2H), 3.78 (s, 2H), 3.61 (t, J = 4.8 Hz, 2H), 3.57- |
| | 3.49 (m, 22H), 3.41 (t, J = 4.6 Hz, 2H), 3.22 (s, 3H), 2.75-2.67 (m, 3H), 2.18 (s, 3H), |
| | 1.99-1.97 (m, 1H), 1.73-1.61 (m, 3H), 1.51-1.44 (m, 1H), 0.99-0.97 (m, 1H) |
| 88 | LC/MS (ESI): m/z = 481.1 [M + 1]⁺. RT = 5.79 min |
| | ¹H NMR (400 MHZ, DMSO-d₆) δ 10.64 (s, 1H), 8.23 (s, 1H), 8.10 (s, 1H), 8.04 (s, 1H), |
| | 7.93-7.84 (m, 4H), 7.59-7.51 (m, 3H), 7.42 (s, 1H), 7.27 (s, 1H), 3.55 (d, J = 13.2 Hz, |
| | 1H), 3.46 (d, J = 13.6 Hz, 1H), 2.76-2.65 (m, 6H), 2.18 (s, 3H), 2.00-1.95 (m, 1H), |
| | 1.74-1.61 (m, 3H), 1.50-1.44 (m, 1H), 1.02-0.95 (m, 1H) |
| 89 | LC/MS (ESI): m/z = 481.1 [M + 1]⁺. RT = 5.86 min |
| | ¹H NMR (400 MHZ, DMSO-d₆) δ 10.82 (s, 1H), 8.78 (d, J = 5.2 Hz, 1H), 8.40 (s, 1H), |
| | 8.07-7.93 (m, 4H), 7.81 (d, J = 7.6 Hz, 2H), 7.40-7.38 (m, 3H), 7.26 (s, 1H), 3.54 (d, J = |
| | 13.2 Hz, 1H), 3.46 (d, J = 13.2 Hz, 1H), 2.75-2.65 (m, 3H), 2.39 (s, 3H), 2.18 (s, 3H), |
| | 2.00-1.95 (m, 1H), 1.73-1.61( m, 3H), 1.52-1.46 (m, 1H), 0.99-0.96 (m, 1H) |
| 90 | LC/MS (ESI): m/z = 459.1 [M + 1]⁺. RT = 5.33 min |
| | ¹H NMR (400 MHZ, DMSO-d₆) δ 8.13 (s, 1H), 7.96 (d, J = 1.6 Hz, 1H), 7.62-7.59 (m, |
| | 2H), 7.42-7.28 (m, 4H), 6.89 (t, J = 6 Hz, 1H), 6.69-6.68 (m, 2H), 6.59 (s, 1H), 4.66 (d, |
| | J = 6 Hz, 2H), 3.37 (d, J = 13.2 Hz, 1H), 3.30 (d, J = 13.2 Hz, 1H), 2.78-2.56 (m, 3H), |
| | 2.13 (s, 3H), 1.87-1.82 (m, 1H), 1.68-1.52( m, 3H), 1.42-1.33 (m, 1H), 1.11-0.85 (m, |
| | 1H) |
| 91 | LC/MS (ESI): m/z = 468.1 [M + 1]⁺. RT = 5.27 min |
| | ¹H NMR (400 MHZ, DMSO-d₆) δ 11.0 (br, 1H), 9.48 (d, J = 1.2 Hz, 1H), 8.62 (d, J = |
| | 0.8 Hz, 1H), 8.34-8.32 (m, 2H), 8.07-8.03 (m, 2H), 7.96 (s, 1H), 7.64-7.60 (m, 3H), |
| | 7.39 (s, 1H), 7.30 (s, 1H), 3.55 (d, J = 13.6 Hz, 1H), 3.47 (d, J = 13.6 Hz, 1H), 2.84- |
| | 2.65 (m, 3H), 2.18 (s, 3H), 2.00-1.95 (m, 1H), 1.74-1.61 (m, 3H), 1.52-1.46 (m, 1H), |
| | 1.14-0.96 (m, 1H) |
| 92 | LC/MS (ESI): m/z = 508.1 [M + 1]⁺. RT = 5.60 min |
| | ¹H NMR (400 MHZ, DMSO-d₆) δ 10.76 (s, 1H), 8.76-8.74 (m, 2H), 8.40 (d, J = 1.2 Hz, |
| | 1H), 8.13 (s, 1H), 8.02-7.95 (m, 3H), 7.89 (d, J = 8.8 Hz, 2H) , 7.38-7.36 (m, 2H), 7.13 |
| | (d, J = 8.8 Hz, 2H), 3.85 (s, 3H), 3.57 (d, J = 13.6 Hz, 1H), 3.49 (d, J = 13.2 Hz, 1H), |
| | 2.77-2.65 (m, 3H), 2.53 (s, 3H), 2.00-1.95 (m, 1H), 1.74-1.61( m, 3H), 1.52-1.46 (m, |
| | 1H), 1.01-0.97 (m, 1H) |
| 93 | LC/MS (ESI): m/z = 484.1 [M + 1]⁺. RT = 5.61 min |
| | ¹H NMR (400 MHZ, DMSO-d₆) δ 10.81 (br, 1H), 8.73 (d, J = 2.4 Hz, 1H), 8.54 (s, 1H), |
| | 8.08 (t, J = 1.6 Hz, 1H), 7.95-7.92 (m, 2H), 7.85-7.83 (m, 2H), 7.54-7.44 (m, 3H) , |
| | 7.38-7.36 (m, 2H), 3.56 (d, J = 13.2 Hz, 1H), 3.48 (d, J = 13.2 Hz, 1H), 2.75-2.64 (m, |
| | 3H), 2.53 (s, 3H), 2.00-1.94 (m, 1H), 1.73-1.60 (m, 3H), 1.48-1.45 ( m, 1H), 1.02-0.92 |
| | (m, 1H) |
| 94 | LC/MS: m/z 481.11 (M + H)⁺ (ES+), 95.09% |
| | ¹H NMR (400 MHZ; DMSO-d₆): δ 1.13-1.18 (m, 1H), 1.42-1.51 (m, 1H), 1.62-1.70 (m, |
| | 1H), 1.73-1.80 (m, 1H), 1.83-1.90 (m, 1H), 2.02-2.12 (m, 1H), 2.15 (s, 3H), 2.57-2.65 |
| | (m, 1H), 2.70-2.75 (m, 1H), 2.80-2.90 (m, 1H), 3.40-3.55 (m, 2H), 3.92 (s, 2H), 4.98 |
| | (bs, 2H), 7.19 (s, 1H), 7.30 (s, 1H), 7.40-7.53 (m, 5H), 7.70-7.75 (m, 2H), 7.83 (s, 1H), |
| | 7.99 (s, 1H), 8.07 (dd, J = 8.4, 2.4 Hz, 1H), 8.81 (d, J = 2 Hz, 1H), 10.50 (bs, 1H) |
| 95 | LC/MS (ESI): m/z = 516.9 [M + 1]⁺. RT = 5.89 min |
| | ¹H NMR (400 MHZ, DMSO-d₆) δ 10.86 (s, 1H), 8.86 (d, J = 5.2 Hz, 1H), 8.46 (d, J = |
| | 0.8 Hz, 1H), 8.08-8.05 (m, 5H), 7.97 (s, 1H), 7.78 (d, J = 8.0 Hz, 2H), 7.40 (s, 1H) , |
| | 7.29-7.01(m, 2H), 3.58-3.45 (m, 2H), 2.83-2.68 (m, 3H), 2.18 (s, 3H), 2.02-1.96 (m, |
| | 1H), 1.74-1.62 (m, 3H), 1.52-1.43 ( m, 1H), 1.00-0.97 (m, 1H) |
| 96 | LC/MS (ESI): m/z = 525.0 [M + 1]⁺. RT = 6.26 min |
| | ¹H NMR (400 MHZ, DMSO-d₆) δ 10.82 (s, 1H), 8.75 (d, J = 5.2 Hz, 1H), 8.38 (d, J = |
| | 1.2 Hz, 1H), 8.07 (d, J = 1.6 Hz, 1H), 8.04 (t, J = 1.8 Hz, 1H), 7.99-7.97 (m, 2H), 7.86 |
| | (d, J = 8.8 Hz, 2H), 7.40 (s, 1H), 7.26 (s, 1H), 7.11 (d, J = 8.8 Hz, 2H), 4.77-4.71 (m, |
| | 1H), 3.54 (d, J = 13.6 Hz, 1H), 3.46 (d, J = 13.6 Hz, 1H), 2.75-2.66 (m, 3H), 2.18 (s, |
| | 3H), 1.97 (t, J = 9.2 Hz, 1H), 1.73-1.61( m, 3H), 1.52-1.43 (m, 1H), 1.31 (d, J = 6.0 Hz, |
| | 6H), 1.01-0.94 (m, 1H) |
| 97 | LC/MS (ESI): m/z = 511.0 [M + 1]⁺. RT = 5.65 min |
| | ¹H NMR (400 MHZ, DMSO-d₆) δ 10.80 (s, 1H), 8.72 (d, J = 5.6 Hz, 1H), 8.51-8.47 (m, |
| | 2H), 8.07-7.91 (m, 4H), 7.39 (s, 1H), 7.26 (s, 1H), 3.54 (d, J = 13.6 Hz, 1H), 3.46 (d, J = |
| | 13.2 Hz, 1H), 2.75-2.58 (m, 6H), 2.18 (s, 3H), 1.99-1.95 (m, 1H), 1.80-1.61 (m, 8H), |
| | 1.51-1.45 (m, 1H), 1.01-0.96 (m, 1H) |
| 98 | LC/MS (ESI): m/z = 537.0 [M + 1]⁺. RT = 6.45 min |
| | ¹H NMR (400 MHZ, DMSO-d₆) δ 10.81 (s, 1H), 8.75 (d, J = 5.2 Hz, 1H), 8.37 (d, J = |
| | 1.2 Hz, 1H), 8.08-8.03 (m, 2H), 7.98-7.96 (m, 2H), 7.85 (d, J = 8.8 Hz, 2H), 7.39 (s, |
| | 1H) , 7.26 (s, 1H), 7.03 (d, J = 8.8 Hz, 2H), 4.81-4.78 (m, 1H), 3.54 (d, J = 13.6 Hz, |
| | 1H), 3.46 (d, J = 13.6 Hz, 1H), 2.75-2.65 (m, 3H), 2.47-2.44 (m, 2H), 2.18 (s, 3H), |
| | 2.10-1.97 (m, 3H), 1.83-1.62 (m, 5H), 1.51-1.44 ( m, 1H), 1.03-0.95 (m, 1H) |
| 99 | LC/MS (ESI): m/z = 503.0 [M + 1]⁺. RT = 5.54 min |
| | ¹H NMR (400 MHZ, DMSO-d₆) δ 10.75 (br, 1H), 8.38 (s, 1H), 8.13-8.08 (m, 3H), 8.01 |
| | (s, 1H), 7.83 (s, 1H), 7.39 (s, 1H), 7.30 (s, 1H), 7.07 (d, J = 8.8 Hz, 2H), 3.83 (s, 3H), |
| | 3.55 (d, J = 13.2 Hz, 1H), 3.46 (d, J = 13.2 Hz, 1H), 2.76-2.67 (m, 3H), 2.18 (s, 3H), |
| | 2.01-1.95 (m, 1H), 1.75-1.61 (m, 3H), 1.49-1.46 (m, 1H), 1.00-0.97 (m, 1H) |
| 100 | LC/MS (ESI): m/z = 492.0 [M + 1]⁺. RT = 5.82 min |
| | ¹H NMR (400 MHZ, DMSO-d₆) δ 10.73 (s, 1H), 8.75 (d, J = 2 Hz, 1H), 8.70 (s, 1H), |
| | 8.12 (t, J = 2 Hz, 1H), 8.01-7.95 (m, 3H), 7.57-7.48 (m, 5H) , 7.38-7.36 (m, 2H), 3.55 |
| | (d, J = 13.6 Hz, 1H), 3.47 (d, J = 13.2 Hz, 1H), 2.77-2.63 (m, 3H), 2.52(s, 3H), 2.39 (s, |
| | 3H), 1.99-1.94 (m, 1H), 1.77-1.60 (m, 3H), 1.51-1.45 (m, 1H), 0.98-0.95 (m, 1H) |
| 101 | LC/MS (ESI): m/z = 526.9 [M + 1]⁺. RT = 4.76 min |
| | ¹H NMR (400 MHZ, DMSO-d₆) δ 10.81 (s, 1H), 8.75 (d, J = 5.2 Hz, 1H), 8.39 (d, J = |
| | 0.8 Hz, 1H), 8.08-7.95 (m, 4H), 7.87 (d, J = 8.8 Hz, 2H) , 7.39 (s, 1H), 7.26 (s, 1H), |
| | 7.13 (d, J = 8.8 Hz, 2H), 4.08 (t, J = 5.2 Hz, 2H), 3.75 (t, J = 4.8 Hz, 2H), 3.54 (d, J = |
| | 13.6 Hz, 1H), 3.50 (d, J = 13.6 Hz, 1H), 2.80-2.65 (m, 3H), 2.19 (s, 3H), 2.00-1.95 (m, |
| | 1H), 1.86-1.61 (m, 3H), 1.52-1.46 (m, 1H), 1.18-0.97 (m, 1H) |
| 102 | LC/MS (ESI): m/z = 517.0 [M + 1]⁺. RT = 5.72 min |
| | ¹H NMR (400 MHZ, DMSO-d₆) δ 10.88 (s, 1H), 8.85 (d, J = 4.8 Hz, 1H), 8.13 (s, 1H), |
| | 8.08-8.04 (m, 2H), 7.95 (s, 1H) , 7.83-7.67 (m, 4H), 7.54-7.52 (m, 1H), 7.39 (s, 1H), |
| | 7.27 (s, 1H), 6.95 (t, J = 14.4 Hz, 1H), 3.54 (d, J = 13.2 Hz, 1H), 3.46 (d, J = 13.2 Hz, |
| | 1H), 2.74-2.67 (m, 3H), 2.18 (s, 3H), 1.98-1.93 (m, 1H), 1.73-1.61 (m, 3H), 1.51-1.46 |
| | (m, 1H), 0.99-0.97 (m, 1H) |
| 103 | LC/MS (ESI): m/z = 539.0 [M + 1]⁺. RT = 5.14 min |
| | ¹H NMR (400 MHZ, DMSO-d₆) δ 10.81 (s, 1H), 8.76 (d, J = 4.4 Hz, 1H), 8.37 (s, 1H), |
| | 8.07-7.92 (m, 4H), 7.87 (d, J = 7.6 Hz, 2H), 7.39 (s, 1H), 7.26 (s, 1H), 6.99 (d, J = 7.6 |
| | Hz, 2H) , 5.42-5.37 (m, 1H), 4.97 (t, J = 6.8 Hz, 2H), 4.58 (dd, J = 7.2, 5.2 Hz, 2H), |
| | 3.54 (d, J = 13.2 Hz, 1H), 3.46 (d, J = 12.8 Hz, 1H), 2.72-2.65 (m, 3H), 2.18 (s, 3H), |
| | 1.98-1.95 (m, 1H), 1.73-1.71 (m, 2H), 1.64-1.61 (m, 1H), 1.52-1.46 (m, 1H), 1.02-0.98 |
| | (m, 1H). |
| 104 | LC/MS (ESI): m/z = 482.0 [M + 1]⁺. RT = 5.10 min |
| | ¹H NMR (400 MHZ, DMSO-d₆) δ 10.78 (br, 1H), 8.97 (s, 1H), 8.06 (d, J = 1.2 Hz, 1H), |
| | 7.99 (s, 1H), 7.84-7.81 (m, 3H), 7.60-7.57 (m, 3H), 7.39 (s, 1H), 7.27 (s, 1H), 3.53 (d, |
| | J = 13.6 Hz, 1H), 3.46 (d, J = 13.2 Hz, 1H), 2.74-2.64 (m, 3H), 2.47 (s, 3H), 2.17 (s, |
| | 3H), 2.01-1.94 (m, 1H), 1.73-1.61 (m, 3H), 1.51-1.42 (m, 1H), 1.00-0.94 (m, 1H) |
| 105 | LC/MS (ESI): m/z = 479.0 [M + 1]⁺. RT = 5.63 min |
| | ¹H NMR (400 MHZ, DMSO-d₆) δ 10.53 (br, 1H), 8.51 (s, 1H), 8.03 (d, J = 1.2 Hz, 1H), |
| | 7.89 (d, J = 1.6 Hz, 1H), 7.56 (s, 1H), 7.35 (s, 1H), 7.24 (s, 1H), 3.52 (d, J = 13.6 Hz, |
| | 1H), 3.43 (d, J = 13.6 Hz, 1H), 3.05-3.00 (m, 1H), 2.74-2.65 (m, 3H), 2.17 (s, 3H), |
| | 2.09-2.06 (m, 2H), 1.97-1.92 (m, 1H), 1.80-1.60 (m, 6H), 1.51-1.35 (m, 5H), 1.29-1.23 |
| | (m, 1H), 1.01-0.93 (m, 1H) |
| 106 | LC/MS (ESI): m/z = 514.0 [M + 1]⁺. RT = 4.85 min |
| | ¹H NMR (400 MHZ, DMSO-d₆) δ 10.67 (s, 1H), 8.27 (d, J = 6.0 Hz, 1H), 8.07 (s, 1H), |
| | 7.99 (s, 1H), 7.94 (s, 1H), 7.55 (d, J = 2.4 Hz, 1H), 7.39 (s, 1H), 7.23 (s, 1H), 7.01 (dd, |
| | J = 5.6, 2.4 Hz, 1H), 3.89-3.86 (m, 2H), 3.53 (d, J = 13.2 Hz, 1H), 3.45 (d, J = 13.6 Hz, |
| | 1H), 3.15-3.12 (m, 2H), 2.74-2.64 (m, 3H), 2.17 (s, 3H), 2.08 (s, 2H), 1.99-1.94 (m, |
| | 1H), 1.81-1.60 (m, 10H), 1.51-1.41 (m, 2H), 1.01-0.93 (m, 1H) |
| 107 | LC/MS (ESI): m/z = 790.9 [M + 1]⁺. RT = 5.30 min |
| | ¹H NMR (400 MHZ, DMSO-d₆) δ 10.81 (s, 1H), 8.75 (d, J = 5.2 Hz, 1H), 8.39 (d, J = |
| | 1.2 Hz, 1H), 8.08-8.04 (m, 2H), 8.00-7.97 (m, 2H), 7.88 (d, J = 8.8 Hz, 2H), 7.40 (s, |
| | 1H), 7.26 (s, 1H), 7.14 (d, J = 8.8 Hz, 2H), 4.19 (t, J = 4.4 Hz, 2H), 3.78 (t, J = 4.4 Hz, |
| | 2H), 3.62-3.38 (m, 26 H), 2.75-2.65 (m, 3H), 2.18 (s, 3H), 2.03-1.95 (m, 1H), 1.75-1.52 |
| | (m, 3H), 1.49-1.46 (m, 1H), 1.03-0.95 (m, 1H) |
| 108 | LC/MS (ESI): m/z = 523 [M + 1]⁺. RT = 6.077 min |
| | ¹H NMR (400 MHZ, DMSO-d₆) δ 10.82 (s, 1H), 8.76 (d, J = 5.2 Hz, 1H), 8.39 (d, J = |
| | 1.2 Hz, 1H), 8.08-8.04 (m, 2H), 7.99-7.93 (m, 2H), 7.89 (d, J = 8.4 Hz, 2H), 7.40 (s, |
| | 1H), 7.28-7.23 (m, 3H), 3.96-3.94 (m, 1H), 3.54 (d, J = 13.2 Hz, 1H), 3.46 (d, J = 13.2 |
| | Hz, 1H), 2.82-2.67 (m, 3H), 2.18 (s, 3H), 2.00-1.95 (m, 1H), 1.84-1.61 (m, 3H), 1.52- |
| | 1.43 (m, 1H), 1.22-0.97 (m, 1H), 0.86-0.82 (m, 2H), 0.76-0.69 (m, 2H) |
| 109 | LC/MS (ESI): m/z = 467.0 [M + 1]⁺. RT = 5.492 min |
| | ¹H NMR (400 MHZ, DMSO-d₆) δ 10.81 (s, 1H), 9.04 (s, 1H), 8.37 (d, J = 8.0 Hz, 1H), |
| | 8.25 (d, J = 8.4 Hz, 1H), 8.08-8.04 (m, 2H), 7.96 (s, 1H), 7.85 (d, J = 7.6 Hz, 2H), |
| | 7.59-7.49 (m, 3H), 7.40 (s, 1H), 7.26 (s, 1H), 3.54 (d, J = 13.2 Hz, 1H), 3.46 (d, J = |
| | 13.6 Hz, 1H), 2.75-2.65 (m, 3H), 2.18 (s, 3H), 2.02-1.95 (m, 1H), 1.74-1.61 (m, 3H), |
| | 1.52-1.43 (m, 1H), 1.02-0.94 (m, 1H) |
| 111 | LC/MS (ESI): m/z = 497.0 [M + 1]⁺. RT = 5.685 min |
| | ¹H NMR (400 MHZ, DMSO-d₆) δ 10.66 (s, 1H), 8.56 (s, 1H), 8.07 (s, 1H), 8.06 (s, 1H), |
| | 8.03-8.01 (m, 1H), 7.93-7.91 (m, 1H), 7.64 (d, J = 6.8 Hz, 2H), 7.54-7.45 (m, 3H), 7.39 |
| | (s, 1H), 7.24 (s, 1H), 4.04 (s, 3H), 3.53 (d, J = 13.2 Hz, 1H), 3.45 (d, J = 13.6 Hz, 1H), |
| | 2.81-2.66 (m, 3H), 2.18 (s, 3H), 1.98-1.91 (m, 1H), 1.83-1.61 (m, 3H), 1.51-1.42 (m, |
| | 1H), 1.16-0.94 (m, 1H) |
| 112 | LC/MS (ESI): m/z = 536 [M + 1]⁺. RT = 6.207 min |
| | ¹H NMR (400 MHZ, DMSO-d₆) δ 10.77 (s, 1H), 8.76-8.74 (m, 2H), 8.38 (d, J = 1.2 Hz, |
| | 1H), 8.13 (s, 1H), 8.02 (s, 1H), 7.98-7.95 (m, 2H),7.86 (d, J = 8.8 Hz, 2H), 7.38-7.36 |
| | (m, 2H), 7.10 (d, J = 8.8 Hz, 2H), 4.77-4.71 (m, 1H), 3.57 (d, J = 13.2 Hz, 1H), 3.49 (d, |
| | J= 13.2 Hz, 1H), 2.77-2.64 (m, 3H), 2.53 (s, 3H), 2.02-1.96 (m, 1H), 1.74-1.61 (m, |
| | 3H), 1.52-1.46 (m, 1H), 1.31 (d, J = 6.0 Hz , 6H), 1.02-0.93 (m, 1H) |
| 113 | LC/MS (ESI): m/z = 490.9 [M + 1]⁺. RT = 5.93 min |
| | ¹H NMR (400 MHZ, DMSO-d₆) δ 10.95 (s, 1H), 8.56 (s, 1H), 8.06 (s, 1H), 8.00 (s, 1H), |
| | 7.86-7.83 (m, 3H), 7.54-7.44 (m, 3H), 7.37 (s, 1H), 7.30 (s, 1H), 4.34-4.19 (m, 1H), |
| | 3.59 (d, J = 13.6 Hz, 1H), 3.51 (d, J = 13.2 Hz, 1H), 2.92-2.72 (m, 3H), 2.22-2.18 (m, |
| | 4H), 2.06-1.93 (m, 2H), 1.73-1.67 (m, 1H) |
| 114 | LC/MS (ESI): m/z = 496.3 [M + 1]⁺. RT = 5.769 min |
| | ¹H NMR (400 MHZ, DMSO-d₆) δ 10.80 (s, 1H), 8.81 (d, J = 5.2 Hz, 1H), 8.77 (d, J = |
| | 2.0 Hz, 1H), 8.43 (d, J = 1.6 Hz, 1H), 8.16 (d, J = 1.6 Hz, 1H), 8.03-8.02 (m, 2H), 7.97 |
| | (dd, J = 8.0, 2.4 Hz, 1H), 7.91 (d, J = 8.4 Hz, 2H), 7.61-7.52 (m, 3H), 7.38-7.36 (m, |
| | 2H), 4.29-4.11 (m, 1H), 3.60 (d, J = 13.2 Hz, 1H), 3.54 (d, J = 13.2 Hz, 1H), 2.85-2.74 |
| | (m, 3H), 2.53 (s, 3H), 2.21-2.16 (m, 1H), 2.04-1.91 (m, 2H), 1.74-1.65 (m, 1H) |
| 115 | LC/MS (ESI): m/z = 531.9 [M + 1]⁺. RT = 7.239 min |
| | ¹H NMR (400 MHZ, DMSO-d₆) δ 10.87 (s, 1H), 9.10 (d, J=1.6 Hz, 1H), 8.82 (d, J = |
| | 4.8 Hz, 1H), 8.44 (d, J = 1.6 Hz, 1H), 8.37 (dd, J = 8.0, 1.6 Hz , 1H), 8.25 (s, 1H), 8.14 |
| | (s, 1H), 8.04 (dd, J = 5.6, 2.0 Hz, 2H), 7.91 (d, J = 7.2 Hz, 2H), 7.61-7.53 (m, 3H), 7.50 |
| | (s, 1H), 3.60 (d, J = 13.6 Hz, 1H), 3.51 (d, J = 13.6 Hz, 1H), 2.78-2.65 (m, 3H), 2.01- |
| | 1.97 (m, 1H), 1.74-1.62 (m, 3H), 1.52-1.43 (m, 1H), 1.02-0.94 (m, 1H) |
| 116 | LC/MS (ESI): m/z = 479.0 [M + 1]⁺. RT = 5.738 min |
| | ¹H NMR (400 MHZ, DMSO-d₆) δ 10.57 (brs, 1H), 9.16 (d, J = 2.0 Hz, 1H), 8.85 (d, J = |
| | 5.2 Hz, 1H), 8.48 (d, J = 1.2 Hz, 1H), 8.37 (dd, J = 8.0, 2.4 Hz , 1H), 8.33 (s, 1H), 8.09 |
| | (dd, J = 5.2, 1.6 Hz , 1H), 7.94-7.92 (m, 2H), 7.75 (s, 1H), 7.62-7.53 (m, 3H), 7.40 (d, J = |
| | 8.0 Hz, 1H), 3.65 (d, J = 14.4 Hz, 1H), 3.58 (d, J = 14.0 Hz, 1H), 2.78-2.70 (m, 3H), |
| | 2.55 (s, 3H), 2.05-1.99 (m, 1H), 1.76-1.64 (m, 3H), 1.56-1.47 (m, 1H), 1.04-0.96 (m, |
| | 1H) |
| 117 | LC/MS (ESI): m/z = 520.9 [M + 1]⁺. RT = 6.824 min |
| | ¹H NMR (400 MHZ, DMSO-d₆) δ 10.91 (s, 1H), 8.82 (d, J = 5.2 Hz, 1H), 8.44 (s, 3H), |
| | 8.14 (s, 1H), 8.10 (s, 1H), 8.05 (dd, J = 4.8, 1.6 Hz, 1H), 7.91 (d, J = 6.8 Hz, 2H), 7.61- |
| | 7.53 (m, 3H), 7.40 (s, 1H), 3.56 (d, J = 13.2 Hz, 1H). 3.49 (d, J = 13.6 Hz, 1H). 2.76- |
| | 2.65 (m, 3H), 2.02-1.95 (m, 1H), 1.75-1.62 (m, 3H), 1.52-1.44 (m, 1H), 1.03-0.94 (m, |
| | 1H) |
| 118 | LC/MS (ESI): m/z = 472.0 [M + 1]⁺. RT = 5.438 min |
| | ¹H NMR (400 MHZ, DMSO-d₆) δ 10.85 (s, 1H), 8.82 (d, J = 5.2 Hz, 1H), 8.43 (d, J = |
| | 1.2 Hz, 1H), 8.08-8.03 (m, 3H), 7.97 (s, 1H), 7.40 (s, 1H), 7.27 (s, 1H), 3.54 (d, J = |
| | 13.6 Hz, 1H). 3.46 (d, J = 13.6 Hz, 1H). 2.75-2.66 (m, 3H), 2.18 (s, 3H), 2.00-1.95 (m, |
| | 1H), 1.74-1.61 (m, 3H), 1.52-1.43 (m, 1H), 1.03-0.94 (m, 1H) |
| 119 | LC/MS (ESI): m/z = 580 [M + 1]⁺. RT = 6.159 min |
| | ¹H NMR (400 MHZ, DMSO-d₆) δ 10.77 (s, 1H), 8.76-8.75 (m, 2H), 8.39 (d, J = 1.6 Hz, |
| | 1H), 8.13 (s, 1H), 8.02 (s, 1H), 7.98-7.95 (m, 2H), 7.88 (d, J = 8.8 Hz, 2H), 7.38-7.36 |
| | (m, 2H), 7.14 (d, J = 8.8 Hz, 2H), 4.16 (t, J = 4.8 Hz, 2H), 3.73 (t, J = 4.8 Hz, 2H), |
| | 3.68-3.62 (m, 1H), 3.57 (d, J = 13.6 Hz, 1H), 3.48 (d, J = 13.2 Hz, 1H), 2.77-2.63 (m, |
| | 3H), 2.53 (s, 3H), 1.99-1.92 (m, 1H), 1.72-1.61 (m, 3H), 1.52-1.43 (m, 1H), 1.12 (d, J = |
| | 6.0 Hz, 6H), 1.01-0.92 (m, 1H) |
| 120 | LC/MS (ESI): m/z = 569.0 [M + 1]⁺. RT = 6.114 min |
| | ¹H NMR (400 MHZ, DMSO-d₆) δ 10.82 (s, 1H), 8.75 (d, J = 5.2 Hz, 1H), 8.39 (d, J = |
| | 1.2 Hz, 1H), 8.08 (d, J = 1.2 Hz, 1H), 8.03 (d, J = 2.0 Hz, 1H), 7.99-7.97 (m, 2H), 7.88 |
| | (d, J = 8.8 Hz, 2H), 7.40 (s, 1H), 7.26 (s, 1H), 7.14 (d, J =8.8 Hz, 2H), 4.16 (t, J = 4.8 |
| | Hz, 2H), 3.73 (t, J = 4.8 Hz, 2H), 3.68-3.62 (m, 1H), 3.54 (d, J = 13.2 Hz, 1H), 3.46 (d, |
| | J = 13.6 Hz, 1H), 2.75-2.66 (m, 3H), 2.18 (s, 3H), 2.00-1.95 (m, 1H), 1.74-1.61 (m, |
| | 3H), 1.52-1.46 (m, 1H), 1.12 (d, J = 6.0 Hz, 3H), 1.02-0.94 (m, 1H). |
| 121 | LC/MS (ESI): m/z = 464 [M + 1]⁺. RT = 5.552 min |
| | ¹H NMR (400 MHZ, DMSO-d₆) δ 10.80 (s, 1H), 8.82 (d, J = 4.8 Hz, 1H), 8.76 (d, J = |
| | 2.0 Hz, 1H), 8.43 (d, J = 1.2 Hz, 1H), 8.13 (s, 1H), 8.04-8.02 (m, 2H), 7.97 (dd, J = 8.0, |
| | 2.4 Hz, 1H), 7.92 (d, J = 8.4 Hz, 2H), 7.62-7.53(m, 3H), 7.38-7.36 (m, 2H), 3.67 (d, J = |
| | 13.2 Hz, 1H), 3.61 (d, J = 13.2 Hz, 1H), 2.74-2.71 (m, 1H), 2.62-2.58 (m, 1H), 2.53- |
| | 2.50 (m, 4H), 2.20-2.16 (m, 1H), 2.08-1.99 (m, 2H), 1.41-1.36 (m, 1H) |
| 122 | LC/MS (ESI): m/z = 514.2 [M + 1]⁺. RT = 6.23 min |
| | ¹H NMR (400 MHZ, DMSO-d₆) δ 10.81 (s, 1H), 8.82 (d, J = 5.2 Hz, 1H), 8.77 (d, J = |
| | 2.0 Hz, 1H), 8.43 (d, J = 1.2 Hz, 1H), 8.18 (s, 1H), 8.04-8.02 (m, 2H), 7.98 (dd, J= 8.0, |
| | 2.4 Hz, 1H), 7.92 (s, 1H), 7.90 (s, 1H), 7.61-7.52 (m, 3H), 7.41-7.37 (m, 2H), 3.67-3.59 |
| | (m, 2H), 2.98-2.92 (m, 1H), 2.72-2.64 (m, 2H), 2.53 (s, 3H), 2.45-2.36 (m, 1H), 2.19- |
| | 2.09 (m, 2H), 1.96-1.87 (m, 1H) |
| 123 | LC/MS (ESI): m/z = 478.0 [M + 1]⁺. RT = 5.570 min |
| | ¹H NMR (400 MHZ, DMSO-d₆) δ 10.79 (s, 1H), 8.82 (d, J = 4.8 Hz, 1H), 8.76 (d, J = |
| | 2.4 Hz, 1H), 8.43 (d, J = 1.2 Hz, 1H), 8.12 (d, J = 1.6 Hz, 1H), 8.04-8.02 (m, 2H), 7.97 |
| | (dd, J = 8.0, 2.4 Hz, 1H), 7.94-7.90 (m, 2H), 7.62-7.53 (m, 3H), 7.38-7.36 (m, 2H), |
| | 3.66 (d, J = 13.6 Hz, 1H). 3.62 (d, J = 13.6 Hz, 1H). 3.11-3.04 (m, 1H), 2.77-2.73 (m, |
| | 1H), 2.56-2.51 (m, 4H), 2.26-2.23 (m, 1H), 2.20 (s, 3H), 2.00-1.91 (m, 1H), 1.55-1.44 |
| | (m, 1H), 1.29-0.78 (m, 2H) |
| 124 | LC/MS (ESI): m/z = 439.0 [M + 1]⁺. RT = 6.07 min |
| | ¹H NMR (400 MHZ, DMSO-d₆) δ 10.84 (s, 1H), 8.82 (d, J = 4.8 Hz, 1H), 8.78 (d, J = |
| | 2.0 Hz, 1H), 8.43 (d, J = 1.2 Hz, 1H), 8.18 (s, 1H), 8.08 (s, 1H), 8.03 (dd, J = 5.2, 2.0 |
| | Hz, 1H), 7.98 (dd, J = 8.0, 2.0 Hz, 1H), 7.92-7.90 (m, 2H), 7.61-7.52 (m, 3H), 7.43 (s, |
| | 1H), 7.38 (d, J = 8.0 Hz, 1H), 4.58 (s, 2H), 3.47 (t, J = 5.6 Hz, 2H), 2.74 (d, J = 5.6 Hz, |
| | 2H), 2.53 (s, 3H) |
| 125 | LC/MS (ESI): m/z = 492.0 [M + 1]⁺. RT = 5.82 min |
| | ¹H NMR (400 MHZ, DMSO-d₆) δ 10.60 (s, 1H), 8.77 (d, J = 2.0 Hz, 1H), 8.23 (d, J = |
| | 1.2 Hz, 1H), 8.14 (s, 1H), 7.99-7.96 (m, 2H), 7.89-7.87 (m, 3H), 7.59-7.50 (m, 3H), |
| | 7.38-7.36 (m, 2H), 3.57 (d, J = 13.2 Hz, 1H), 3.49 (d, J = 13.6 Hz, 1H), 2.78-2.65 (m, |
| | 6H), 2.53 (s, 3H), 1.96 (t, J = 10.0 Hz, 1H), 1.73-1.61 (m, 3H), 1.52-1.43 (m, 1H), 1.01- |
| | 0.93 (m, 1H) |
| 126 | LC/MS (ESI): m/z = 510.0 [M + 1]⁺. RT = 6.026 min |
| | ¹H NMR (400 MHZ, DMSO-d₆) δ 10.75 (s, 1H), 8.76 (d, J = 2.0 Hz, 1H), 8.70 (s, 1H), |
| | 8.15 (s, 1H), 8.02 (s, 1H), 7.98-7.95 (m, 2H), 7.57-7.48 (m, 5H), 7.38-7.36 (m, 2H), |
| | 4.28-4.10 (m, 1H), 3.59 (d, J = 13.6 Hz, 1H), 3.52 (d, J = 13.2 Hz, 1H), 2.84-2.75 (m, |
| | 3H), 2.52 (s, 3H), 2.39 (s, 3H), 2.20-2.14 (m, 1H), 2.03-1.90 (m, 2H), 1.70-1.64 (m, |
| | 1H) |
| 127 | LC/MS (ESI): m/z = 452.0 [M + 1]⁺. RT = 5.66 min |
| | ¹H NMR (400 MHZ, CD₃OD) δ 8.20 (s, 1H), 8.03 (s, 1H), 7.94-7.92 (m, 2H), 7.86 (d, J = |
| | 7.6 Hz, 1H), 7.72-7.70 (m, 3H), 7.61 (t, J = 8.0 Hz, 1H), 7.50-7.46 (m, 2H), 7.40-7.37 |
| | (m, 1H), 7.33 (s, 1H), 7.30 (s, 1H), 3.74 (d, J = 13.2 Hz, 1H), 3.68 (d, J = 13.2 Hz, 1H), |
| | 3.55-3.48 (m, 1H), 2.85-2.78 (m, 2H), 2.60-2.55 (m, 1H), 2.45 (dd, J = 9.6, 4.4 Hz, |
| | 1H), 2.28-2.19 (m, 4H), 1.64-1.56 (m, 1H) |
| 128 | LC/MS (ESI): m/z = 594 [M + 1]⁺. RT = 6.395 min |
| | ¹H NMR (400 MHZ, DMSO-d₆) δ 10.71 (s, 1H), 8.75 (d, J = 2.0 Hz, 1H), 8.66 (s, 1H), |
| | 8.12 (s, 1H), 8.00 (s, 1H), 7.97-7.94 (m, 2H), 7.45 (d, J = 8.8 Hz, 2H), 7.38-7.35 (m, |
| | 2H), 7.10 (d, J = 8.8 Hz, 2H), 4.15 (t, J = 4.8 Hz, 2H), 3.73 (t, J = 4.8 Hz, 2H), 3.68- |
| | 3.62 (m, 1H), 3.55 (d, J = 13.2 Hz, 1H), 3.48 (d, J = 13.6 Hz, 1H), 2.76-2.62 (m, 3H), |
| | 2.52 (s, 3H), 2.40 (s, 3H), 1.99-1.92 (m, 1H), 1.73-1.60 (m, 3H), 1.51-1.42 (m, 1H), |
| | 1.13 (d, J = 6.4 Hz, 6H), 1.02-0.93 (m, 1H) |
| 129 | LC/MS (ESI): m/z = 583.3 [M + 1]⁺. RT = 6.36 min |
| | ¹H NMR (400 MHZ, DMSO-d₆) δ 10.76 (s, 1H), 8.65 (s, 1H), 8.06 (d, J = 1.2 Hz, 1H), |
| | 8.02 (s, 1H), 7.95 (s, 2H), 7.45 (d, J = 8.8 Hz, 2H), 7.39 (s, 1H), 7.25 (s, 1H), 7.10 (d, J = |
| | 8.8 Hz, 2H), 4.15 (t, J = 4.8 Hz, 2H), 3.73 (t, J = 4.8 Hz, 2H), 3.68-3.62 (m, 1H), 3.53 |
| | (d, J = 13.2 Hz, 1H), 3.45 (d, J= 13.2 Hz, 1H), 2.74-2.64 (m, 3H), 2.40 (s, 3H), 2.18 (s, |
| | 3H), 2.01-1.94 (m, 1H), 1.74-1.59 (m, 3H), 1.51-1.42 (m, 1H), 1.13 (d, J = 6.4 Hz, 6H), |
| | 1.04-0.96 (m, 1H) |
| 130 | LC/MS (ESI): m/z = 528.2 [M + 1]⁺. RT = 6.51 min |
| | ¹H NMR (400 MHZ, DMSO-d₆) δ 10.75 (s, 1H), 8.76 (d, J = 2.0 Hz, 1H), 8.70 (s, 1H), |
| | 8.16 (s, 1H), 8.03 (s, 1H), 7.98-7.96 (m, 2H), 7.57-7.48 (m, 5H), 7.40-7.36 (m, 2H), |
| | 3.65-3.58 (m, 2H), 2.95-2.90 (m, 1H), 2.71-2.64 (m, 2H), 2.52 (s, 3H), 2.39-2.33 (m, |
| | 4H), 2.19-2.12 (m, 2H), 1.96-1.84 (m, 1H) |
| 131 | LC/MS (ESI): m/z = 582.2 [M + 1]⁺. RT = 8.76 min |
| | ¹H NMR (400 MHZ, DMSO-d₆) δ 10.82 (s, 1H), 9.10 (d, J = 2.0 Hz, 1H), 8.71 (s, 1H), |
| | 8.37 (dd, J = 8.0, 2.0 Hz, 1H), 8.28 (s, 1H), 8.13 (s, 1H), 8.03 (d, J = 8.4 Hz, 1H), 7.98 |
| | (s, 1H), 7.58-7.48 (m, 6H), 3.65 (m, 2H), 2.99-2.91 (m, 1H), 2.72-2.63 (m, 2H), 2.45- |
| | 2.36 (m, 4H), 2.23-2.13 (m, 2H), 1.98-1.89 (m, 1H) |
| 132 | LC/MS (ESI): m/z = 497.3 [M + 1]⁺. RT = 5.80 min |
| | ¹H NMR (400 MHZ, DMSO-d₆) δ 10.73 (s, 1H), 8.75 (d, J = 2.0 Hz, 1H), 8.70 (s, 1H), |
| | 8.12 (s, 1H), 8.01 (s, 1H), 7.98-7.94 (m, 2H), 7.38-7.36 (m, 2H), 3.56 (d, J = 13.2 Hz, |
| | 1H), 3.47 (d, J = 13.2 Hz, 1H), 2.77-2.63 (m, 3H), 2.52 (s, 3H), 2.39 (s, 3H), 2.00-1.94 |
| | (m, 1H), 1.73-1.60 (m, 3H), 1.51-1.42 (m, 1H), 1.01-0.93 (m, 1H) |
| 133 | LC/MS (ESI): m/z = 630.3 [M + 1]⁺. RT = 7.09 min |
| | ¹H NMR (400 MHZ, DMSO-d₆) δ 10.73 (s, 1H), 8.77 (d, J = 2.4 Hz, 1H), 8.66 (s, 1H), |
| | 8.16 (s, 1H), 8.02 (s, 1H), 7.98-7.95 (m, 2H), 7.45 (d, J = 8.8 Hz, 2H), 7.39-7.36 (m, |
| | 2H), 7.10 (d, J = 8.4 Hz, 2H), 4.15 (t, J = 4.8 Hz, 2H), 3.73 (t, J = 4.8 Hz, 2H), 3.68- |
| | 3.61 (m, 3H), 2.98-2.89 (m, 1H), 2.71-2.63 (m, 2H), 2.52 (s, 3H), 2.40 (s, 3H), 2.20- |
| | 2.12 (m, 2H), 1.98-1.86 (m, 1H). 1.76-1.70 (m, 1H), 1.13 (d, J = 6.0 Hz, 6H) |
| 134 | LC/MS (ESI): m/z = 501.3 [M + 1]⁺. RT = 5.74 min |
| | ¹H NMR (400 MHZ, DMSO-d₆) δ 10.81 (s, 1H), 8.82 (d, J = 5.2 Hz, 1H), 8.77 (d, J = |
| | 2.0 Hz, 1H), 8.44 (d, J = 1.6 Hz, 1H), 8.16 (s, 1H), 8.04-8.02 (m, 2H), 7.97 (dd, J = 8.0, |
| | 2.4 Hz, 1H), 7.39-7.36 (m, 2H), 4.28-4.10 (m, 1H), 3.60 (d, J = 13.2 Hz, 1H), 3.54 (d, J = |
| | 13.2 Hz, 1H), 2.85-2.74 (m, 3H), 2.53 (s, 3H), 2.21-2.16 (m, 1H), 2.04-1.91 (m, 2H), |
| | 1.72-1.65 (m, 1H) |
| 135 | LC/MS (ESI): m/z = 493.2 [M + 1]⁺. RT = 6.31 min |
| | ¹H NMR (400 MHZ, DMSO-d₆) δ 10.73 (s, 1H), 8.75 (s, 1H ), 8.70 (s, 1H ), 8.12 (s, |
| | 1H), 8.00-7.95 (m, 3H), 7.55-7.50 (m, 5H), 7.38-7.36 (m, 2H), 4.58 (d, J = 4.4 Hz, 1H), |
| | 3.61-3.47 (m, 3H), 2.84 (d, J = 8.0 Hz, 1H), 2.70 (d, J = 10.0 Hz, 1H), 2.52 (s, 3H), |
| | 2.39 (s, 3H), 1.92 (t, J = 10.4 Hz, 1H), 1.81-1.73 (m, 2H), 1.64-1.61 (m, 1H), 1.50-1.41 |
| | (m, 1H), 1.12-1.03 (m, 1H) |
| 136 | LC/MS (ESI): m/z = 493.2 [M + 1]⁺. RT = 6.06 min |
| | ¹H NMR (400 MHZ, DMSO-d₆) δ 10.52 (brs, 1H), 9.15 (s, 1H), 8.73 (s, 1H), 8.37 (d, J = |
| | 8.0 Hz, 1H), 8.29 (s, 1H), 8.01 (s, 1H), 7.73 (s, 1H), 7.58-7.49 (m, 5H), 7.39 (d, J = |
| | 8.8 Hz, 1H), 3.63 (d, J = 14.4 Hz, 1H), 3.56 (d, J = 14.8 Hz, 1H), 2.76-2.68 (m, 3H), |
| | 2.55 (s, 3H), 2.40 (s, 3H), 2.04-1.98 (m, 1H), 1.78-1.63 (m, 3H), 1.54-1.45 (m, 1H), |
| | 1.03-0.94 (m, 1H) |
| 137 | LC/MS (ESI): m/z = 511.3 [M + 1]⁺. RT = 6.425 min |
| | ¹H NMR (400 MHZ, DMSO-d₆) δ 10.75 (s, 1H), 8.76 (d, J = 2.0 Hz, 1H), 8.70 (s, 1H), |
| | 8.14 (s, 1H), 8.01 (s, 1H), 7.98-7.95 (m, 2H), 7.57-7.48 (m, 5H), 7.38-7.36 (m, 2H), |
| | 5.16 (d, J = 5.2 Hz, 1H), 4.33-4.14 (m, 1H), 3.64-3.51 (m, 3H), 2.88-2.78 (m, 2H), 2.52 |
| | (s, 3H), 2.39 (s, 3H), 2.11-2.06 (m, 1H), 1.99-1.98 (m, 1H), 1.89 (t, J = 10.0 Hz, 1H), |
| | 1.70-1.60 (m, 1H) |
| 138 | LC/MS (ESI): m/z = 565.2 [M + 1]⁺. RT = 8.68 min |
| | ¹H NMR (400 MHZ, DMSO-d₆) δ 10.82 (s, 1H), 9.09 (s, 1H), 8.70 (s, 1H), 8.36 (d, J = |
| | 8.4 Hz, 1H), 8.26 (s, 1H), 8.12 (s, 1H), 8.03 (d, J = 8.0 Hz, 1H), 7.98 (s, 1H), 7.57-7.50 |
| | (m, 6H), 5.17 (d, J = 5.2 Hz, 1H), 4.33-4.16 (m, 1H), 3.66-3.54 (m, 3H), 2.91-2.77 (m, |
| | 2H), 2.39 (s, 3H), 2.13-2.05 (m, 1H), 2.00-1.89 (m, 2H), 1.73-1.62 (m, 1H) |
| 139 | LC/MS (ESI): m/z = 598.3 [M + 1]⁺. RT = 6.41 min |
| | ¹H NMR (400 MHZ, DMSO-d₆) δ 10.78 (s, 1H), 8.77-8.75 (m, 2H), 8.39 (d, J = 1.2 Hz, |
| | 1H), 8.16 (s, 1H), 8.03 (s, 1H), 7.99-7.96 (m, 2H), 7.88 (d, J = 8.8 Hz, 2H), 7.38-7.36 |
| | (m, 2H), 7.14 (d, J = 8.8 Hz, 2H), 4.28-4.15 (m, 3H), 3.73 (t, J = 4.8 Hz, 2H), 3.68-3.52 |
| | (m, 3H), 2.85-2.73 (m, 3H), 2.53 (s, 3H), 2.18 (t, J = 9.8 Hz, 1H), 2.04-1.91 (m, 2H), |
| | 1.68-1.61 (m, 1H), 1.12 (t, J = 6.0 Hz, 6H) |
| 140 | LC/MS (ESI): m/z = 568.2 [M + 1]⁺. RT = 8.46 min |
| | ¹H NMR (400 MHZ, DMSO-d₆) δ 10.88 (s, 1H), 9.11 (d, J = 2.0 Hz, 1H), 8.83 (d, J = |
| | 5.2 Hz, 1H), 8.44 (d, J = 1.2 Hz, 1H), 8.38 (dd, J = 8.4, 2.0 Hz, 1H), 8.29 (s, 1H), 8.15 |
| | (s, 1H), 8.04 (dd, J = 5.6, 2.0 Hz, 2H), 7.93-7.90 (m, 2H), 7.62-7.53 (m, 4H), 3.69-3.62 |
| | (m, 2H), 3.01-2.92 (m, 1H), 2.73-2.65 (m, 2H), 2.45-2.39 (m, 1H), 2.24-2.11 (m, 2H), |
| | 1.99-1.87 (m, 1H) |
| 141 | LC/MS (ESI): m/z = 595.4 [M + 1]⁺. RT = 6.67 min |
| | ¹H NMR (400 MHZ, DMSO-d₆) δ 10.51 (brs, 1H), 9.15 (d, J = 2.0 Hz, 1H), 8.69 (s, 1H), |
| | 8.36 (dd, J = 8.4, 2.4 Hz, 1H), 8.30 (s, 1H), 8.00 (s, 1H), 7.73 (s, 1H), 7.47 (d, J = 8.8 |
| | Hz, 2H), 7.40 (d, J = 8.0 Hz, 1H), 7.11 (d, J = 8.4 Hz, 2H), 4.15 (t, J = 4.8 Hz, 2H), |
| | 3.73 (t, J = 4.4 Hz, 2H), 3.69-3.54 (m, 3H), 2.76-2.67 (m, 3H), 2.55 (s, 3H), 2.41 (s, |
| | 3H), 2.03-1.97 (m, 1H), 1.77-1.72 (m, 2H), 1.66-1.63 (m, 1H), 1.54-1.46 (m, 1H), 1.13 |
| | (d, J = 6.0 Hz, 6H), 1.03-0.95 (m, 1H) |
| 142 | LC/MS (ESI): m/z = 497.2 [M + 1]⁺. RT = 6.33 min |
| | ¹H NMR (400 MHZ, DMSO-d₆) δ 10.59 (s, 1H), 9.15 (d, J = 5.2 Hz, 1H ), 8.86-8.82 (m, |
| | 1H), 8.48-8.45 (m, 1H), 8.38-8.30 (m, 2H), 8.09-8.05 (m, 1H), 7.91 (m, 2H), 7.76-7.73 |
| | (m, 1H), 7.59-7.56 (m, 3H), 7.43-7.37 (m, 1H), 4.31-4.16 (m, 1H), 3.67-3.59 (m, 2H), |
| | 2.86-2.77 (m, 3H), 2.54 (s, 3H), 2.24 (t, J = 10.0 Hz, 1H), 2.02-1.97 (m, 2H), 1.77-1.69 |
| | (m, 1H) |
| 143 | LC/MS (ESI): m/z = 515.2 [M + 1]⁺. RT = 6.58 min |
| | ¹H NMR (400 MHZ, DMSO-d₆) δ 10.61 (s, 1H), 9.18 (d, J = 2.0 Hz, 1H), 8.85 (d, J = |
| | 4.8 Hz, 1H), 8.46 (d, J = 1.2 Hz, 1H), 8.39 (dd, J =8.0, 2.4 Hz, 1H), 8.29 (s, 1H), 8.08 |
| | (dd, J = 5.2, 2.0 Hz, 1H), 7.93-7.91 (m, 2H), 7.82 (s, 1H), 7.62-7.53 (m, 3H), 7.41 (d, J = |
| | 8.0 Hz, 1H), 3.80 (J = 14.4 Hz, 1H), 3.75 (J = 14.4 Hz, 1H), 3.60-3.57 (m, 1H), 2.88- |
| | 2.76 (m, 2H), 2.55-2.52 (m, 4H), 2.45-2.40 (m, 1H), 2.34-2.24 (m, 1H), 2.17-2.09 (m, |
| | 1H) |
| 144 | LC/MS (ESI): m/z = 530.3 [M + 1]⁺. RT = 5.43 min |
| | ¹H NMR (400 MHZ, DMSO-d₆) δ 10.77 (s, 1H), 9.98 (s, 1H), 8.77 (d, J = 2.0 Hz, 1H), |
| | 8.72 (d, J = 5.2 Hz, 1H), 8.36 (d, J = 1.2 Hz, 1H), 8.17 (s, 1H), 8.03 (s, 1H), 7.97 (dd, J = |
| | 8.0, 2.4 Hz, 1H), 7.93 (dd, J = 5.2, 2.0 Hz, 1H), 7.77 (d, J = 8.8 Hz, 2H), 7.40-7.36 |
| | (m, 2H), 6.95 (d, J = 8.4 Hz, 2H), 3.66-3.59 (m, 2H), 3.00-2.91 (m, 1H), 2.72-2.64 (m, |
| | 2H), 2.53 (s, 3H), 2.43-2.37 (m, 1H), 2.21-2.10 (m, 2H), 2.00-1.89 (m, 1H) |
| 145 | LC/MS (ESI): m/z = 670.3 [M + 1]⁺. RT = 8.95 min |
| | ¹H NMR (400 MHZ, DMSO-d₆) δ 10.85 (s, 1H), 9.11 (d, J = 1.6 Hz, 1H), 8.76 (d, J = |
| | 5.2 Hz, 1H), 8.40-8.36 (m, 2H), 8.29 (s, 1H), 8.14 (s, 1H), 8.03 (d, J = 8.4 Hz, 1H), |
| | 7.99 (d, J = 5.2 Hz, 1H), 7.88 (d, J = 8.8 Hz, 2H), 7.54 (s, 1H), 7.14 (d, J = 8.8 Hz, |
| | 2H), 4.16 (t, J = 4.8 Hz, 2H), 3.73 (t, J =4.6 Hz, 2H), 3.69-3.62 (m, 3H), 2.99-2.92 (m, |
| | 1H), 2.73-2.65 (m, 2H), 2.46-2.39 (m, 1H), 2.24-2.14 (m, 2H), 2.00-1.89 (m, 1H), 1.12 |
| | (d, J = 6.0 Hz, 6H) |
| 146 | LC/MS (ESI): m/z = 617.3 [M + 1]⁺. RT = 6.85 min |
| | ¹H NMR (400 MHZ, DMSO-d₆) δ 10.78 (s, 1H), 8.78-8.75 (m, 2H), 8.39 (d, J = 1.6 Hz, |
| | 1H), 8.18 (s, 1H), 8.03 (s, 1H), 7.99-7.96 (m, 2H), 7.88 (d, J = 8.8 Hz, 2H), 7.41 (s, |
| | 1H), 7.37 (d, J = 8.14 Hz, 1H), 7.13 (d, J = 8.4 Hz, 2H), 4.16 (t, J = 4.8 Hz, 2H), 3.73 |
| | (t, J = 4.8 Hz, 2H), 3.68-3.59 (m, 3H), 3.00-2.91 (m, 1H), 2.72-2.64 (m, 2H), 2.53 (s, |
| | 3H), 2.43-2.37 (m, 1H), 2.22-2.10 (m, 2H), 1.97-1.89 (m, 1H), 1.13 (d, J = 6.0 Hz, 6H) |
| 147 | LC/MS (ESI): m/z = 514.2 [M + 1]⁺. RT = 6.26 min |
| | ¹H NMR (400 MHZ, DMSO-d₆) δ 10.81 (s, 1H), 8.82 (d, J = 5.2 Hz, 1H), 8.78 (d, J = |
| | 2.0 Hz, 1H), 8.43 (d, J = 1.2 Hz, 1H), 8.18 (s, 1H), 8.04-8.02 (m, 2H), 7.98 (dd, J = 8.0, |
| | 2.4 Hz, 1H), 7.92-7.89 (m, 2H), 7.61-7.52 (m, 3H), 7.41 (s, 1H), 7.38 (d, J = 8.0 Hz, |
| | 1H), 3.67-3.59 (m, 2H), 3.03-2.94 (m, 1H), 2.73-2.65 (m, 2H), 2.53 (s, 3H), 2.43-2.37 |
| | (m, 1H), 2.23-2.11 (m, 2H), 2.00-1.90 (m, 1H) |
| 148 | LC/MS (ESI): m/z = 509.3 [M + 1]⁺. RT = 5.35 min |
| | ¹H NMR (400 MHZ, DMSO-d₆) δ 10.51 (brs, 1H), 9.15 (d, J = 2.0 Hz, 1H), 8.67 (s, 1H), |
| | 8.37 (dd, J = 8.0, 2.4 Hz, 1H), 8.30 (s, 1H), 7.99 (s, 1H), 7.73 (s, 1H), 7.41-7.35 (m, |
| | 3H), 6.92 (d, J = 8.4 Hz, 2H), 3.63 (d, J = 14.4 Hz, 1H), 3.56 (d, J = 14.4 Hz, 1H), |
| | 2.73-2.67 (m, 3H), 2.55 (s, 3H), 2.42 (s, 3H), 2.04-1.98 (m, 1H), 1.78-1.71 (m, 2H), |
| | 1.67-1.61 (m, 1H), 1.54-1.48 (m, 1H), 1.03-0.97 (m, 1H) |
| 149 | LC/MS (ESI): m/z = 494.3 [M + 1]⁺. RT = 4.865 min |
| | ¹H NMR (400 MHZ, DMSO-d₆) δ 10.74 (s, 1H), 8.76 (d, J = 2.0 Hz, 1H), 8.72 (d, J = |
| | 5.2 Hz, 1H), 8.36 (d, J = 1.6 Hz, 1H), 8.12 (s, 1H), 8.01 (s, 1H), 7.96 (dd, J = 8.0, 2.4 |
| | Hz, 1H), 7.93 (dd, J = 5.2, 2.0 Hz, 1H), 7.77 (d, J = 8.4 Hz, 2H), 7.38-7.36 (m, 2H), |
| | 6.94 (d, J = 8.8 Hz, 2H), 3.57 (d, J = 13.2 Hz, 1H), 3.48 (d, J = 13.6 Hz, 1H), 2.78-2.63 |
| | (m, 3H), 2.53 (s, 3H), 2.01-1.95 (m, 1H), 1.74-1.61 (m, 3H), 1.52-1.43 (m, 1H), 1.02- |
| | 0.93 (m, 1H) |
| 150 | LC/MS (ESI): m/z = 493.2 [M + 1]⁺. RT = 8.39 min |
| | ¹H NMR (400 MHZ, DMSO-d₆) δ 10.90 (s, 1H), 9.11 (d, J = 2.0 Hz, 1H), 8.83 (d, J = |
| | 5.2 Hz, 1H), 8.44 (d, J = 1.6 Hz, 1H), 8.38 (dd, J = 8.0, 1.6 Hz, 1H), 8.29 (s, 1H), 8.18 |
| | (s, 1H), 8.05-8.03 (m, 2H), 7.91 (d, J = 7.2 Hz, 2H), 7.62-7.54 (m, 4H), 4.61 (s, 2H), |
| | 3.48 (t, J = 5.8 Hz, 2H), 2.75 (t, J = 5.8 Hz, 2H) |
| 151 | LC/MS (ESI): m/z = 532.2 [M + 1]⁺. RT = 6.34 min |
| | ¹H NMR (400 MHZ, DMSO-d₆) δ 10.80 (s, 1H), 8.81 (d, J = 4.8 Hz, 1H), 8.77 (d, J = |
| | 2.4 Hz, 1H), 8.42 (d, J = 1.6 Hz, 1H), 8.17 (d, J = 1.6 Hz, 1H), 8.04-7.96 (m, 5H), 7.44- |
| | 7.36 (m, 4H), 3.66-3.59 (m, 2H), 2.99-2.91 (m, 1H), 2.72-2.64 (m, 2H), 2.53 (s, 3H), |
| | 2.43-2.37 (m, 1H), 2.22-2.10 (m, 2H), 1.99-1.87 (m, 1H) |
| 152 | LC/MS (ESI): m/z = 496.3 [M + 1]⁺. RT = 5.675 min |
| | ¹H NMR (400 MHZ, DMSO-d₆) δ 10.78 (s, 1H), 8.80 (d, J = 5.2 Hz, 1H), 8.76 (d, J = |
| | 2.0 Hz, 1H), 8.42 (s, 1H), 8.13 (s, 1H), 8.02-7.95 (m, 5H), 7.44-7.36 (m, 4H), 3.57 (d, J = |
| | 13.6 Hz, 1H), 3.49 (d, J = 13.2 Hz, 1H), 2.77-2.64 (m, 3H), 2.53 (s, 3H), 1.99-1.94 |
| | (m, 1H), 1.76-1.61 (m, 3H), 1.51-1.42 (m, 1H), 1.02-0.93 (m, 1H) |
| 153 | LC/MS (ESI): m/z = 467.2 [M + 1]⁺. RT = 5.48 min |
| | ¹H NMR (400 MHZ, DMSO-d₆) δ 10.84 (s, 1H), 8.81 (d, J = 5.2 Hz, 1H), 8.43 (d, J = |
| | 1.2 Hz, 1H), 8.08-8.02 (m, 3H), 7.97-7.90 (m, 3H), 7.61-7.52 (m, 3H), 7.39 (s, 1H), |
| | 7.28-7.27 (m, 1H), 3.54 (d, J = 13.6 Hz, 1H), 3.46 (d, J = 13.6 Hz, 1H), 2.83-2.65 (m, |
| | 3H), 2.18 (s, 3H), 2.02-1.95 (m, 1H), 1.87-1.61 (m, 3H), 1.52-1.43 (m, 1H), 1.17-0.95 |
| | (m, 1H) |
| 154 | LC/MS (ESI): m/z = 503.2 [M + 1]⁺. RT = 6.26 min |
| | ¹H NMR (400 MHZ, DMSO-d₆) δ 10.85 (s, 1H), 8.82 (d, J = 5.2 Hz, 1H), 8.43 (s, 1H), |
| | 8.09 (s, 1H), 8.08 (s, 1H), 8.04 (dd, J = 4.8, 1.6 Hz, 1H), 7.98 (s, 1H), 7.92 (s, 1H), |
| | 7.90 (s, 1H), 7.61-7.52 (m, 3H), 7.40 (s, 1H), 7.31 (d, J = 4.8 Hz, 1H), 3.64-3.57 (m, |
| | 2H), 2.99-2.91 (m, 1H), 2.70-2.61 (m, 2H), 2.40-2.33 (m, 1H), 2.18-2.09 (m, 5H), 1.99- |
| | 1.88 (m, 1H) |

### Example 9. In Vitro PCSK9-LDLR Binding Assay

Compounds were assayed for their ability to inhibit the binding between PCSK9 and the LDL receptor using a CircuLex PCSK9-LDLR in vitro binding assay kit. The procedure employed the reagents and buffers included in the kit as follows. 88 μL of 1× reaction buffer was placed into each well. 5 μL of test compound in 20% DMSO was added into each well. 10 mM solutions of test compounds in DMSO were diluted by 3-fold series to give 6-point concentration curves. The compounds were then diluted 20-fold with the reaction buffer. To each well was then added 7 μL of His-tagged PCSK9 wild type solution (1000 ng/mL). The plate was then covered with a plate sealer and incubated at room temperature for 3 hours, shaking at 300 rpm on an orbital microplate shaker. The test solutions were washed 4 times with 350 μL wash buffer. One 100 μL of biotinylated anti-His-tag monoclonal antibody was added to each well. The plate was covered with a plate sealer, and incubated at room temperature for 1 h, shaking at 300 rpm. The test solutions were washed 4 times with 350 μL wash buffer. 10 μL of HRP-conjugated streptavidin was added to each well. The plate was covered with a plate sealer, and incubated at room temperature for 20 min, shaking at 300 rpm. The test solutions were washed 4 times with 350 μL wash buffer. 100 μL of substrate reagent was added into each well. The plate was covered with a plate sealer, and incubated at room temperature for 15 min, shaking at 300 rpm. Finally, 100 μL of stop solution was added to each well in the same order as the previously added substrate reagent. Absorbance was measured at 450 nm and 540 nm and IC₅₀ curves were plotted.

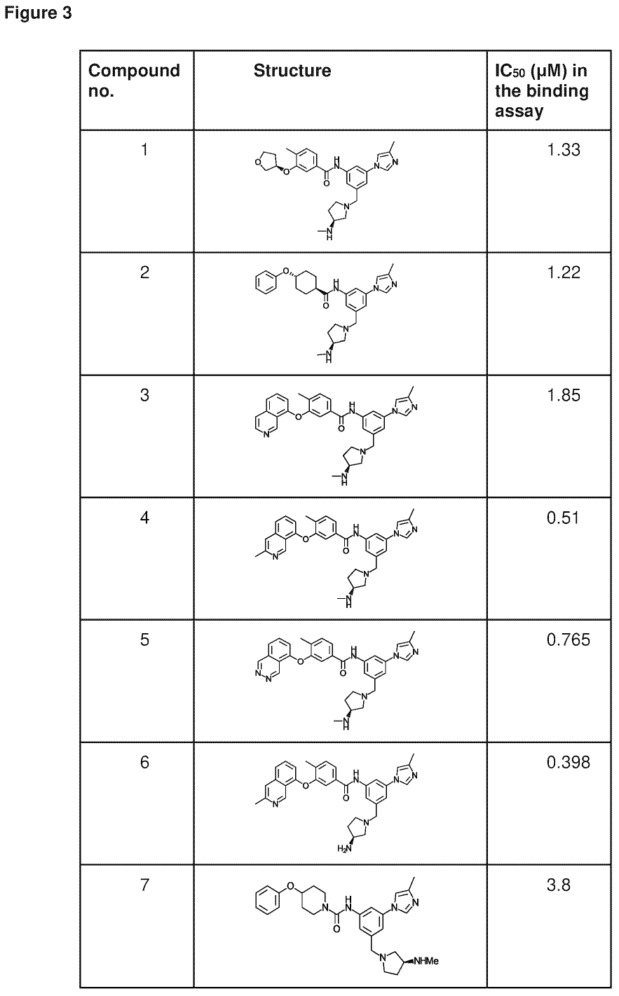

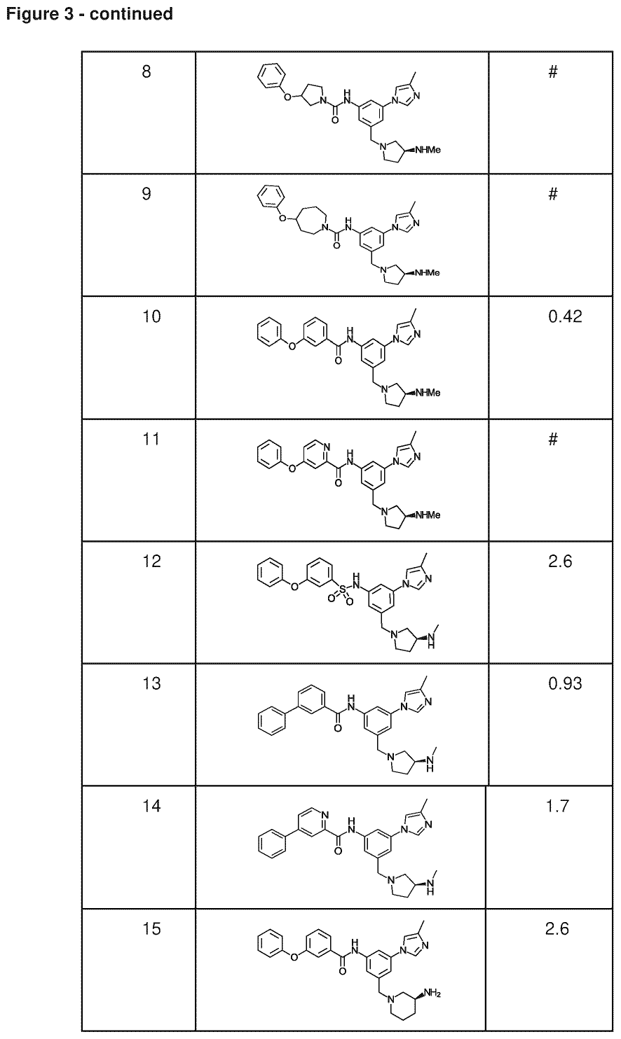

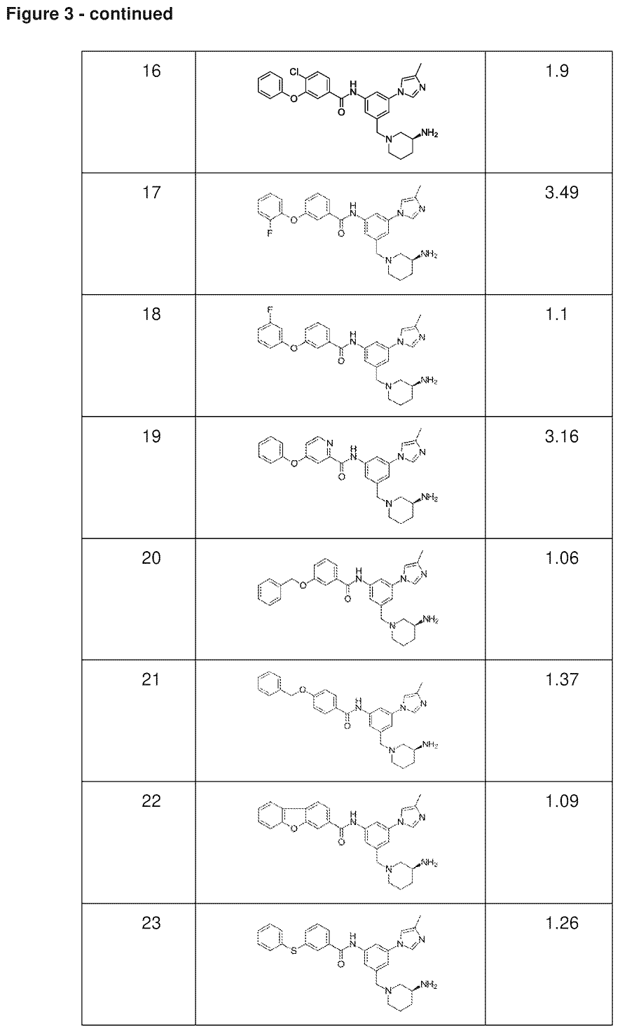

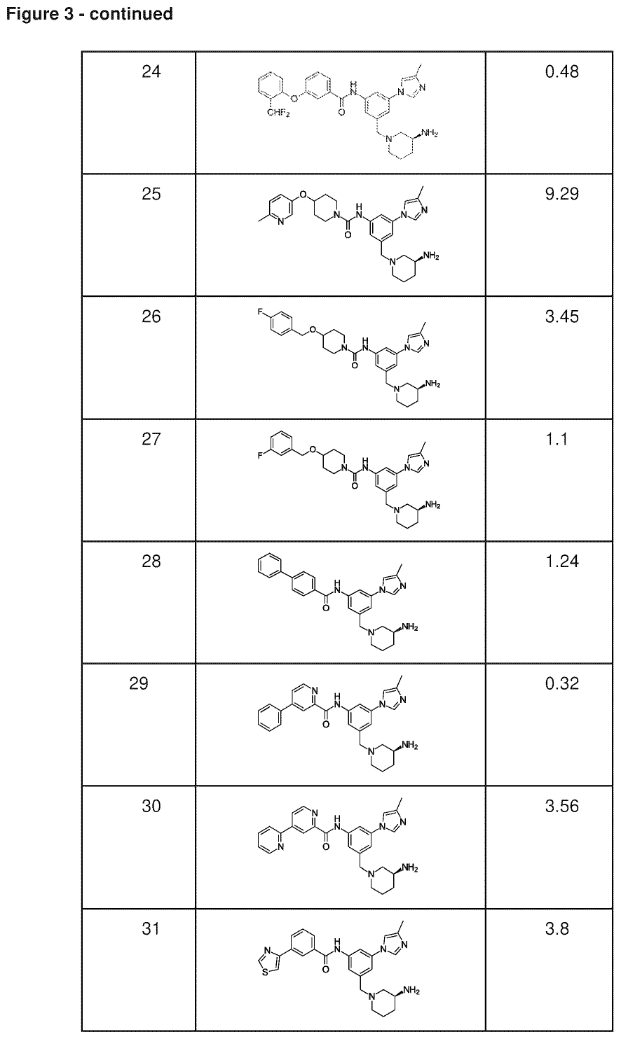

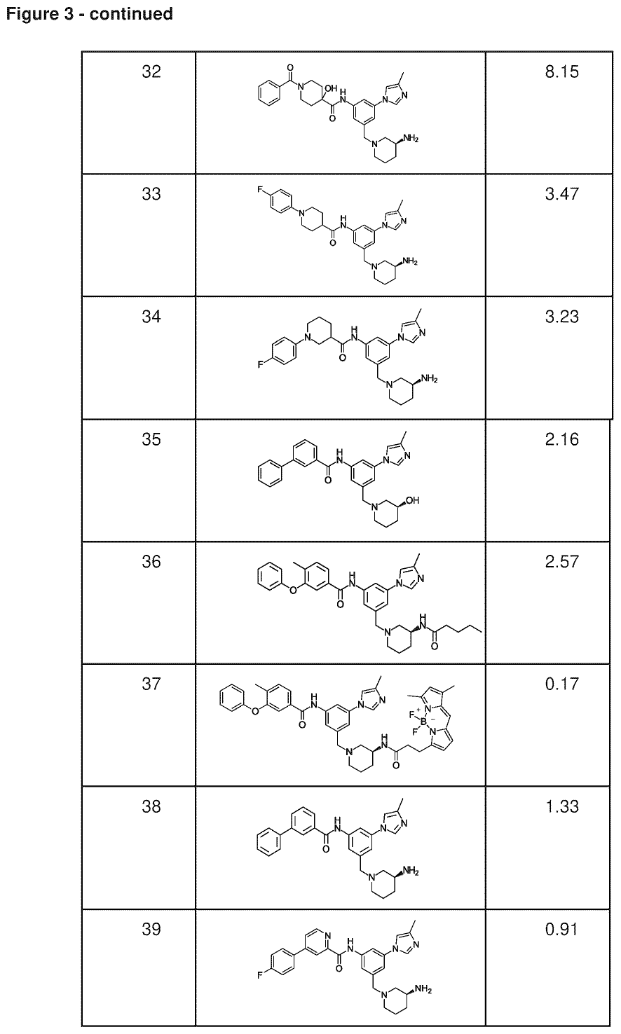

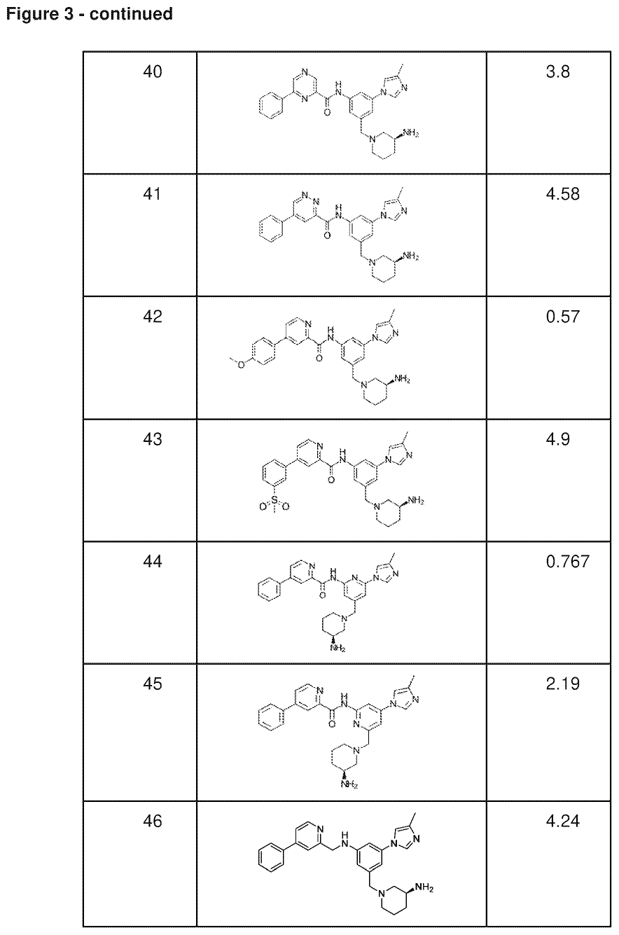

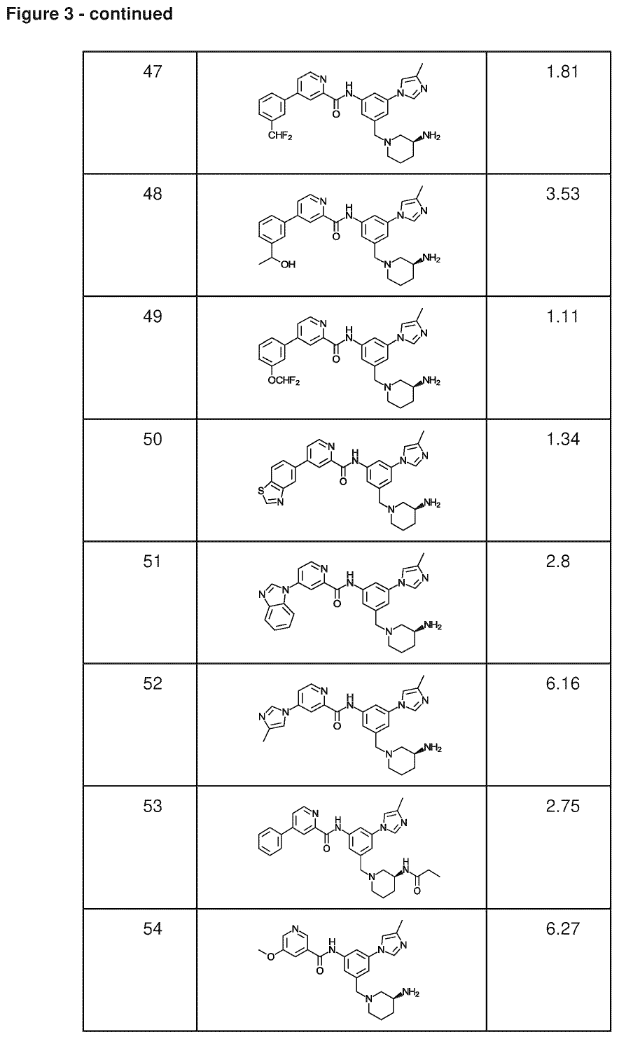

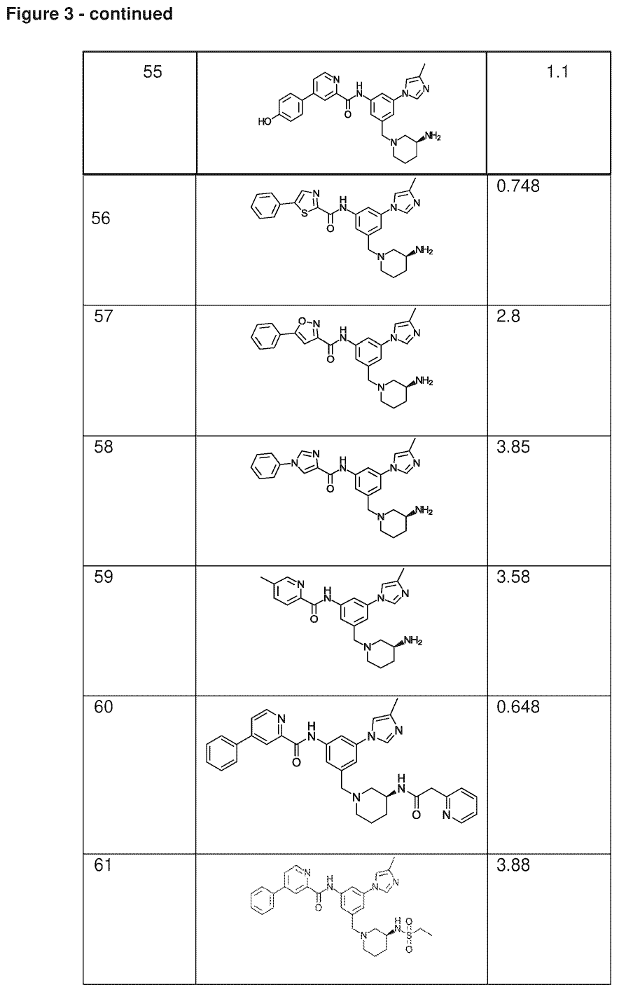

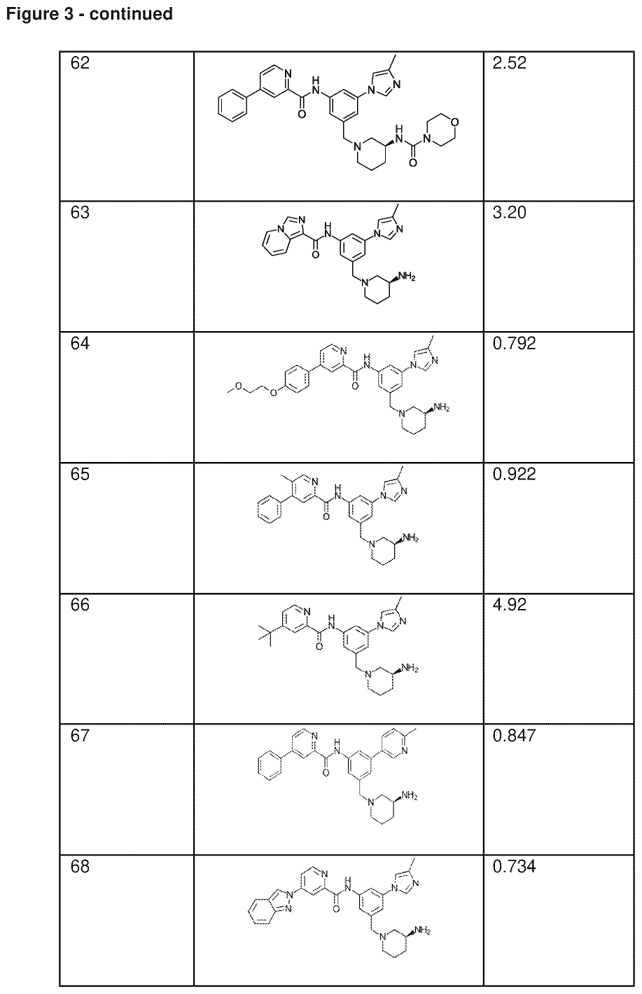

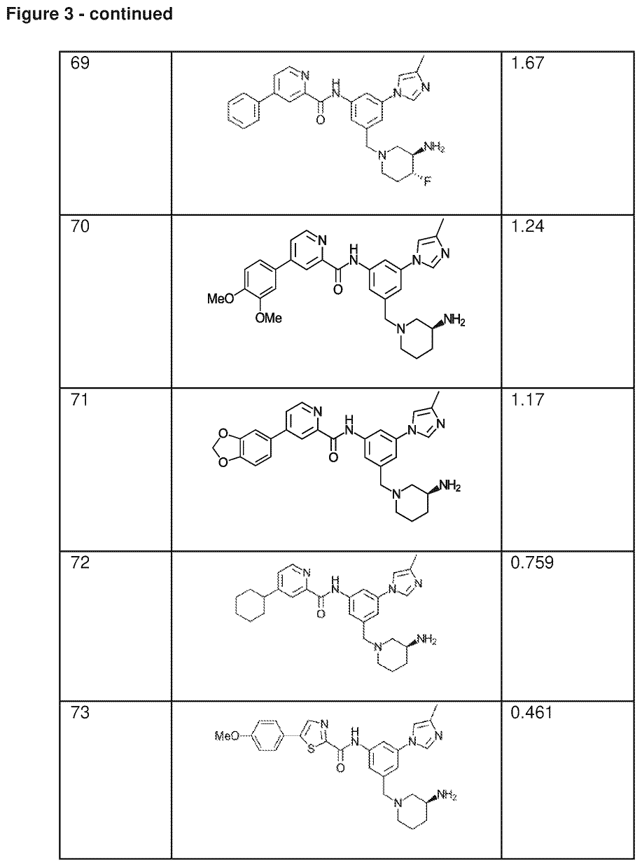

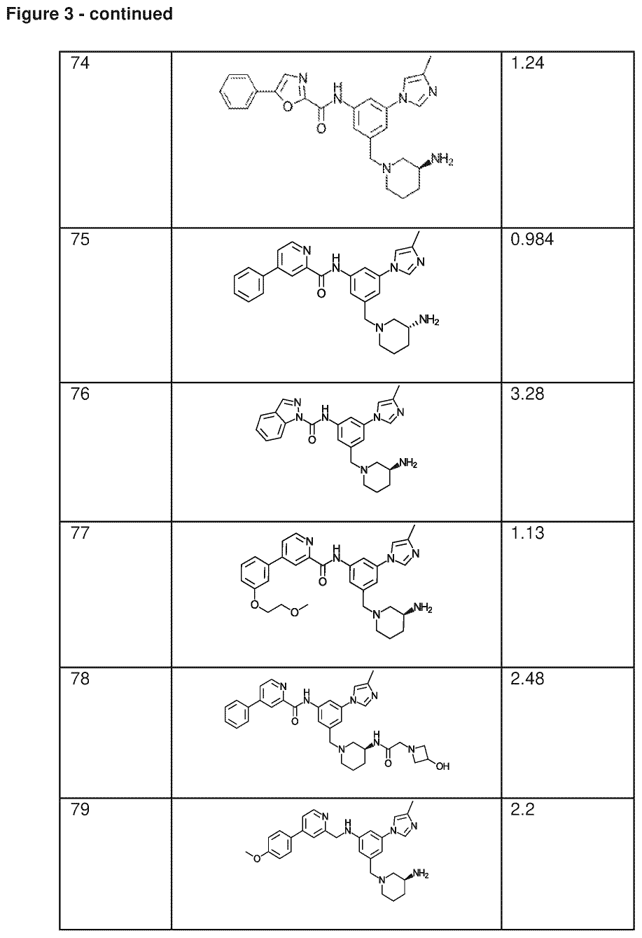

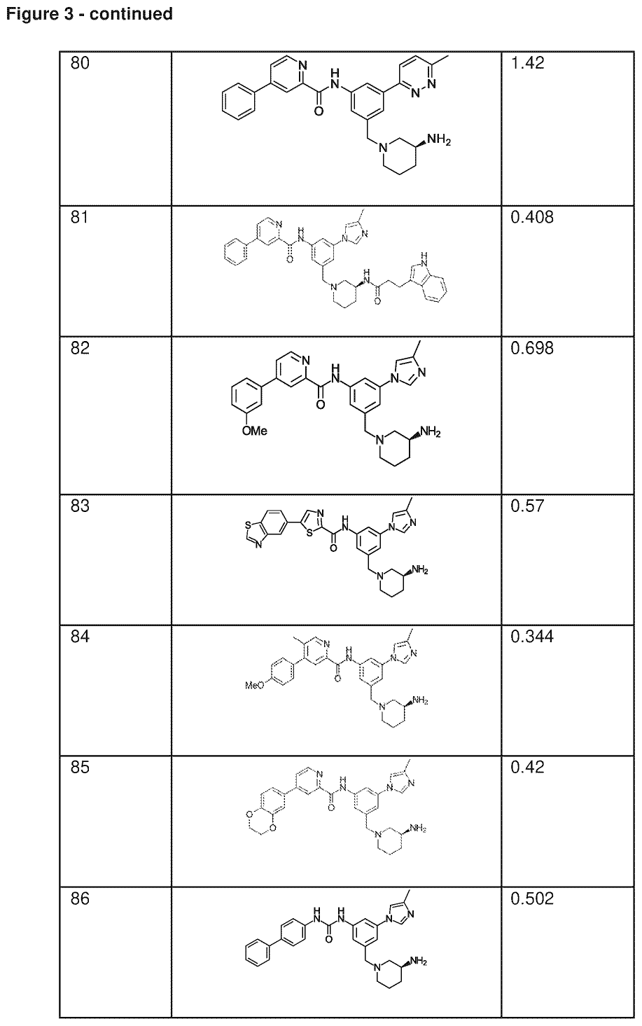

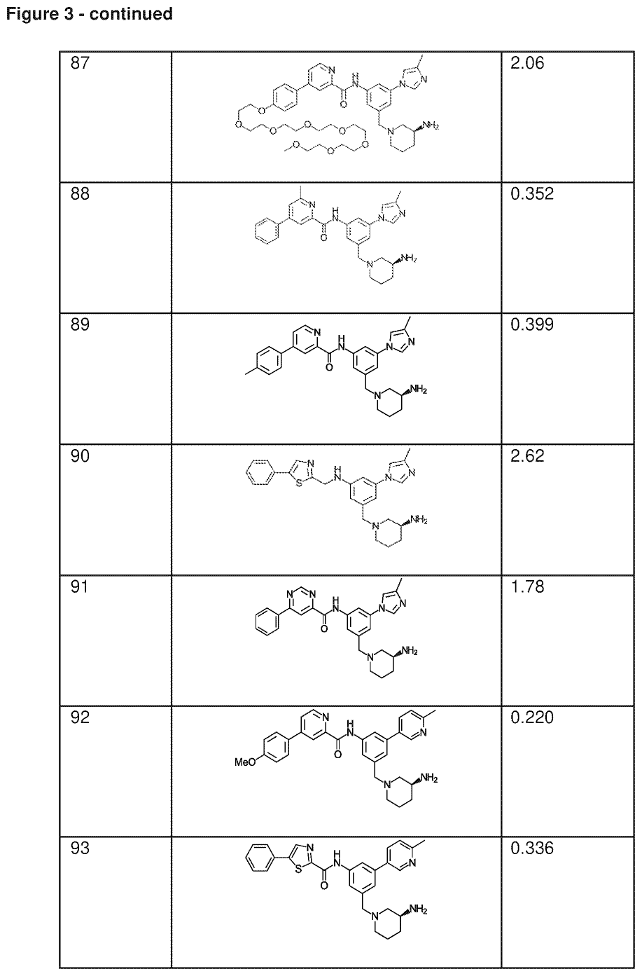

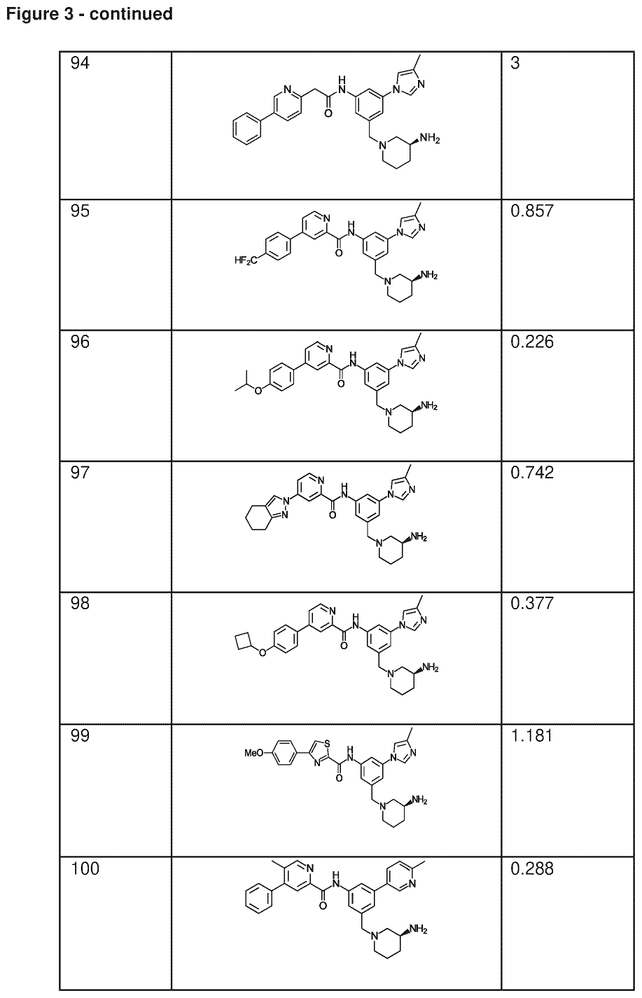

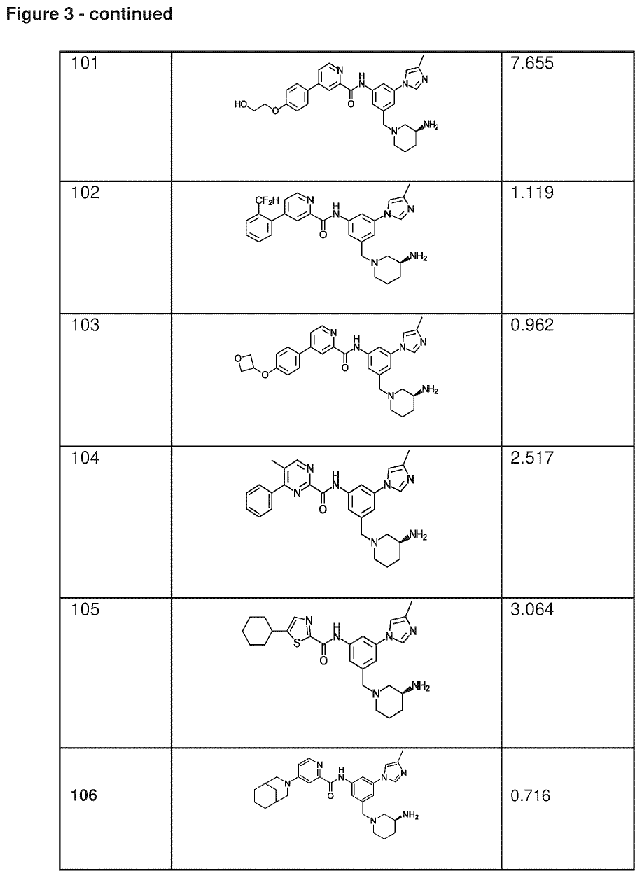

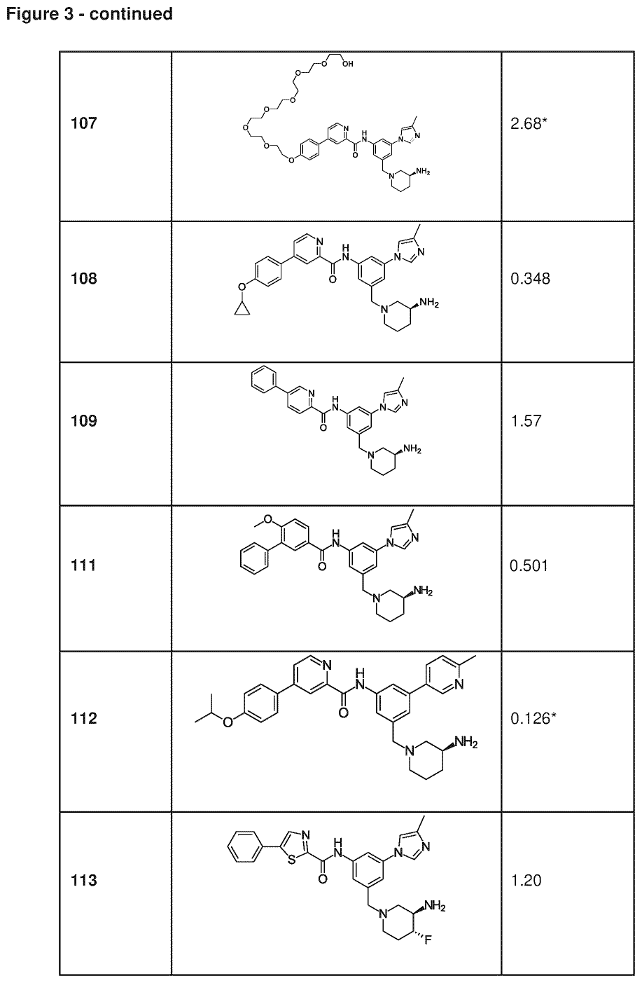

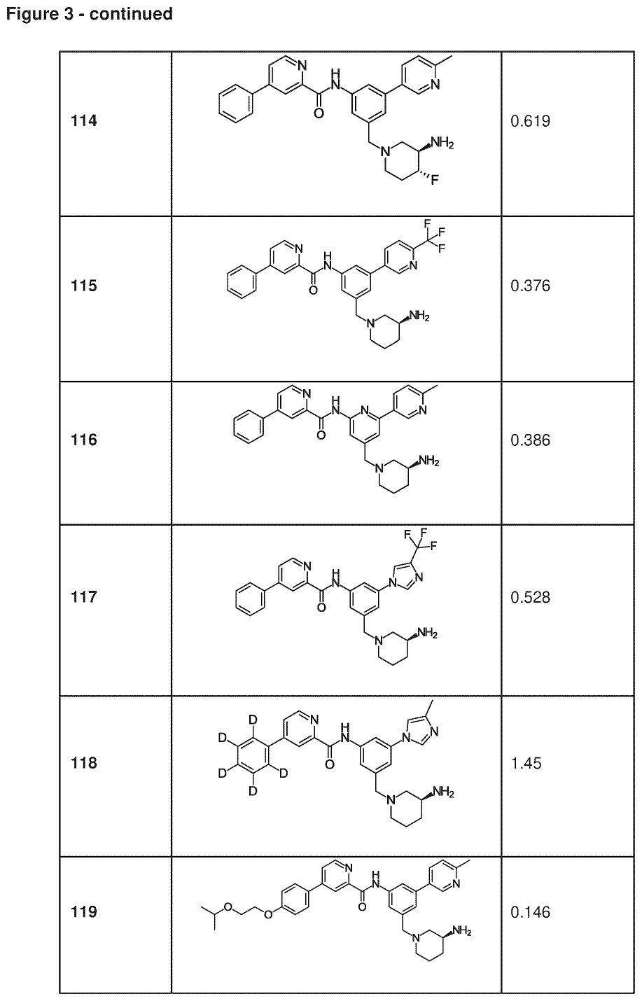

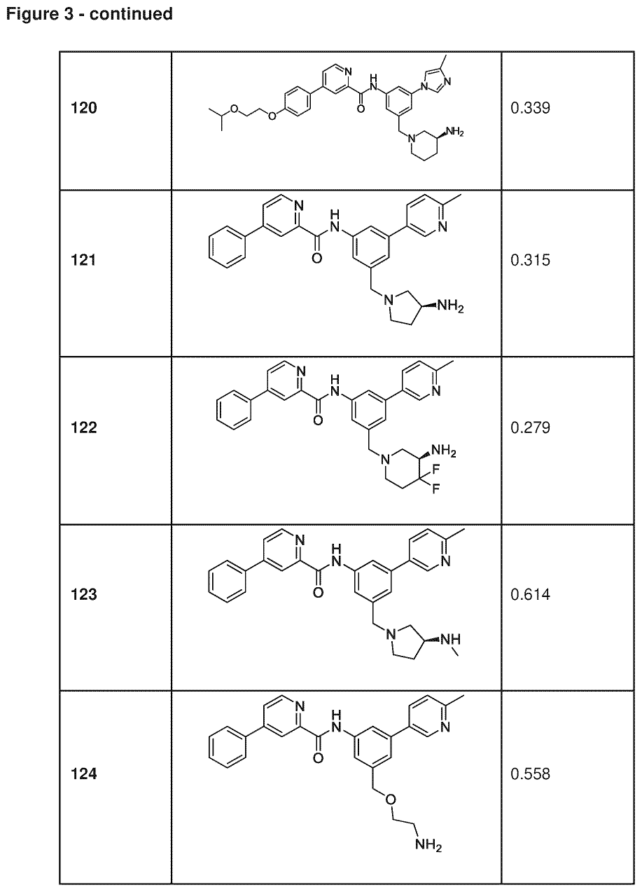

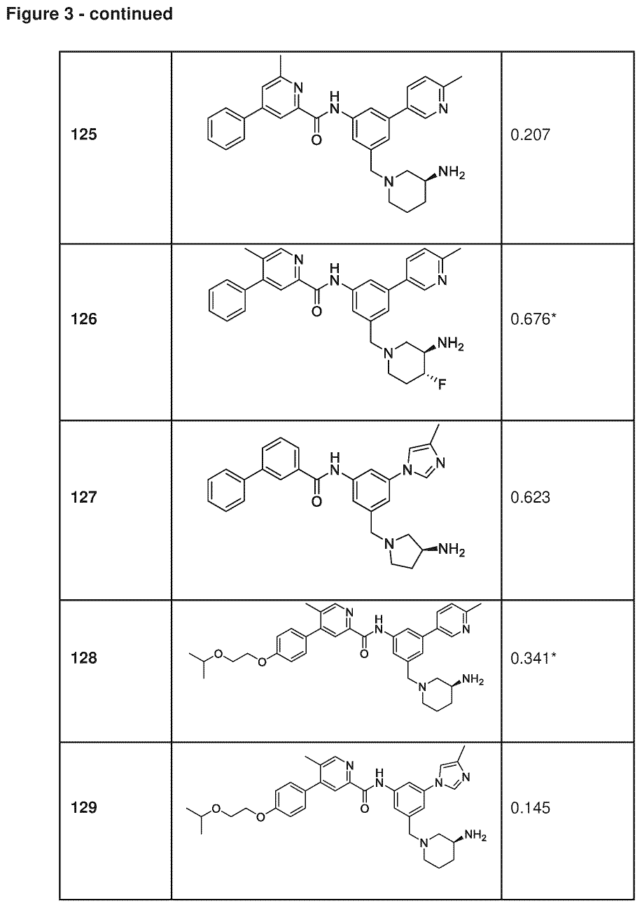

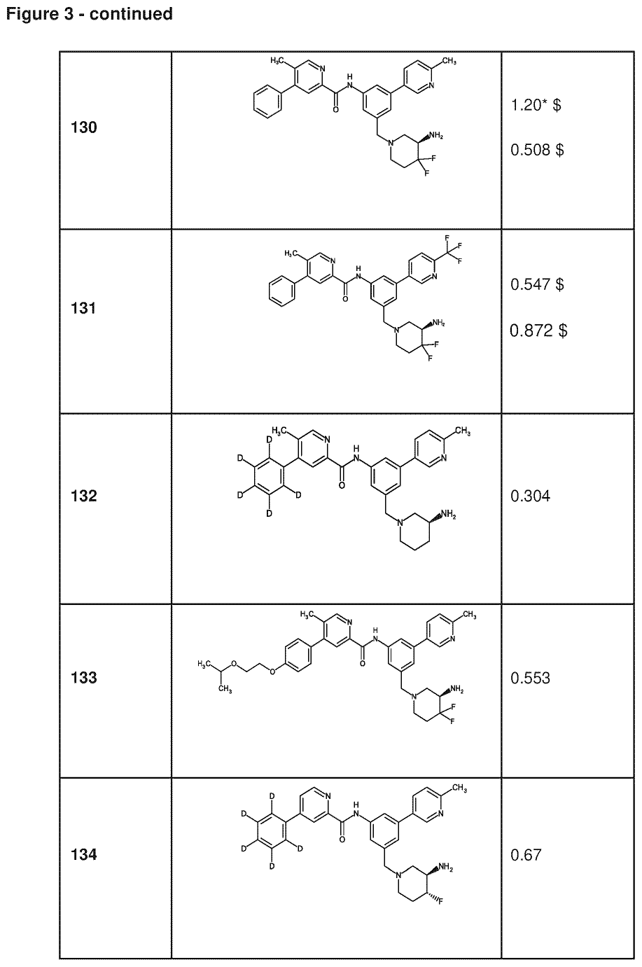

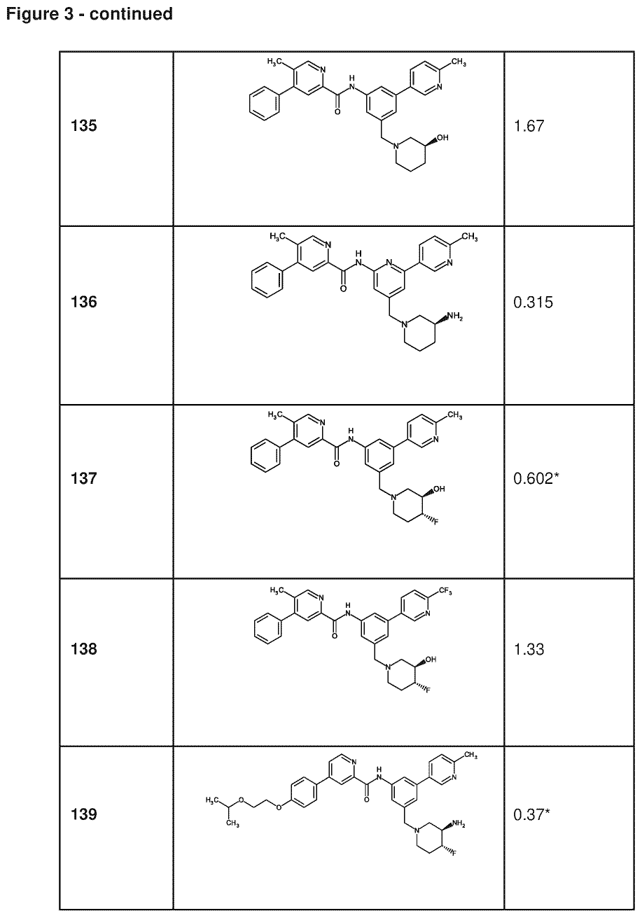

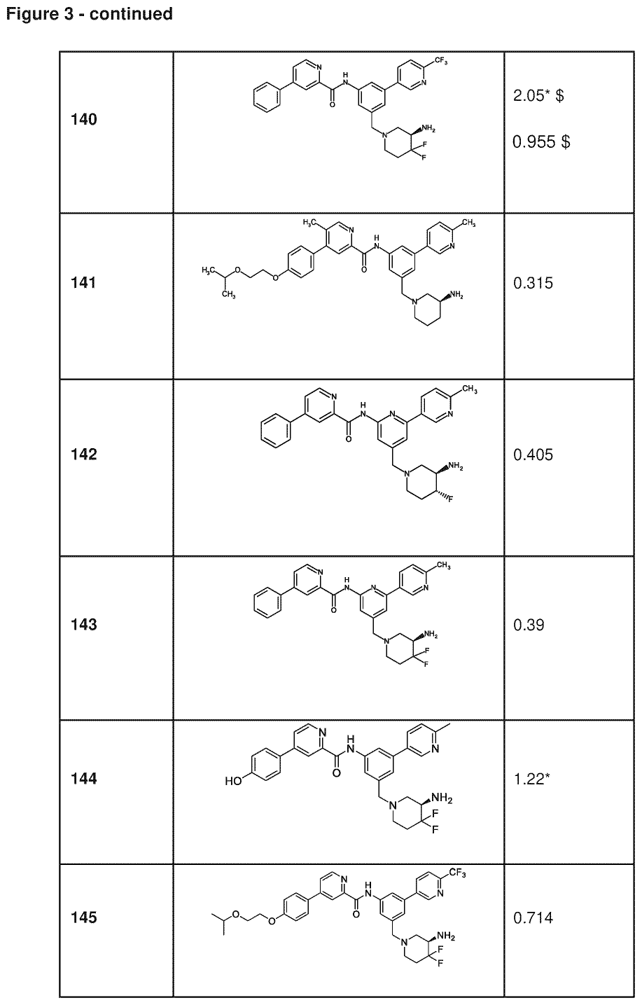

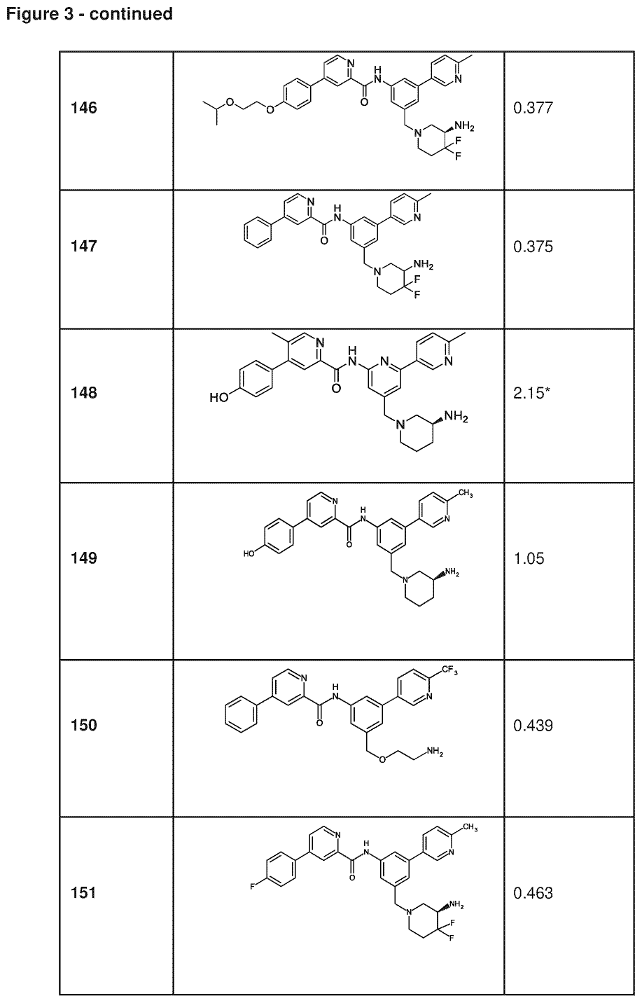

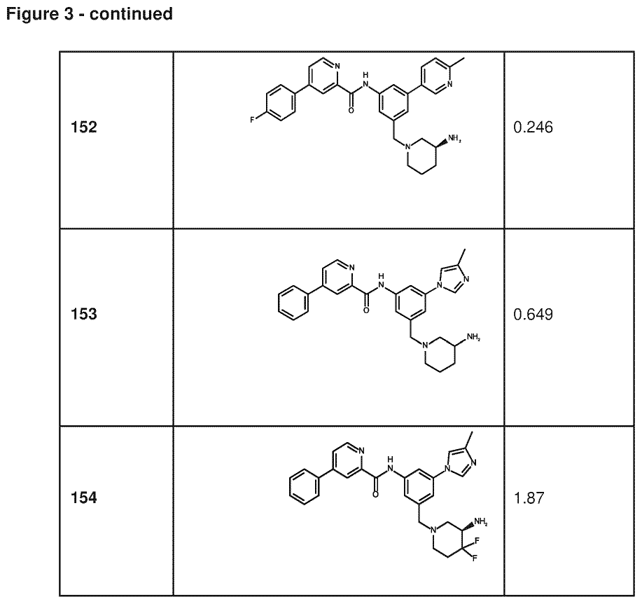

The results of the binding assay are shown in FIG. 3, reported as IC₅₀ (μM).

### Example 10. Ex Vivo LDLR Surface Expression on Primary Human Lymphocytes—Comparative Example

Peripheral blood mononuclear cells (PBMCs) were isolated using Ficoll and subsequently frozen at −80° C. in RPMI culture medium containing 70% fetal calf serum (FCS) and 10% DMSO until use. Freshly thawed PBMCs were seeded in flat bottom 96-well plates (2×10⁵ cells per well) in RPMI containing 10 mM HEPES, 1 mM sodium pyruvate and 0.5% FCS for 2 h at 37° C. The culture medium was subsequently supplemented with or without 600 ng/mL of recombinant PCSK9-D374Y for 4 h in the presence or absence of increasing concentrations of the test compounds or the control—alirocumab. Alirocumab is a fully humanized monoclonal antibody that binds free plasma PCSK9.

Cells were subsequently washed in ice cold phosphate buffered saline (PBS) containing 1% bovine serum albumin (BSA) and incubated with an allophycocyanin-conjugated antibody against the human LDLR or an IgG1 isotype control for 20 min at room temperature in the dark. Cells were washed twice in ice-cold PBS-1% BSA and once in ice-cold PBS. PBMCs were analysed on a Cytoflex flow cytometer. Forward scatter versus side-scatter gates were set to include only viable lymphocytes. A minimum of 5000 lymphocytes were analysed for LDLR expression. Mean fluorescence intensity (MFI) of cells incubated with the isotype control fluorescent antibody (non-specific binding) were subtracted from the MFI of cells incubated with a specific anti-LDLR fluorescent antibody to determine specific levels of LDLR cell surface expression [ΔMFU].

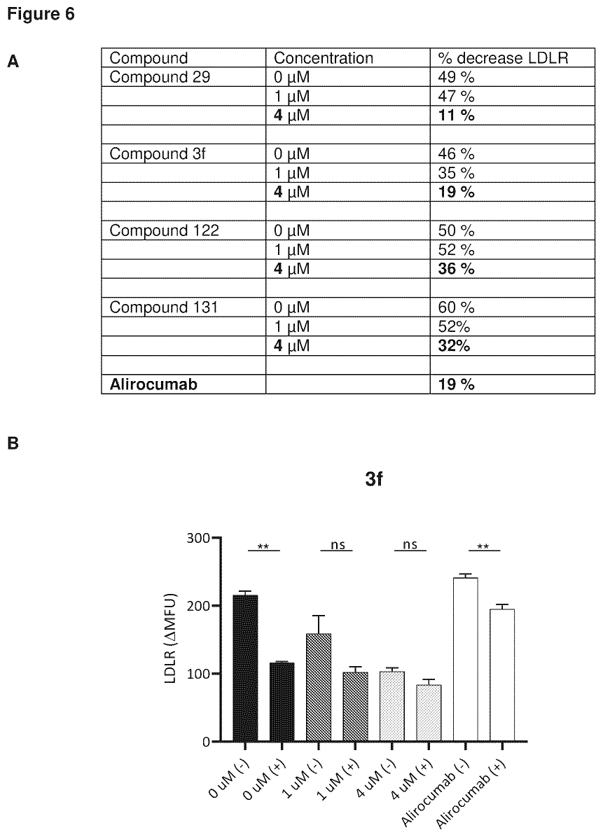

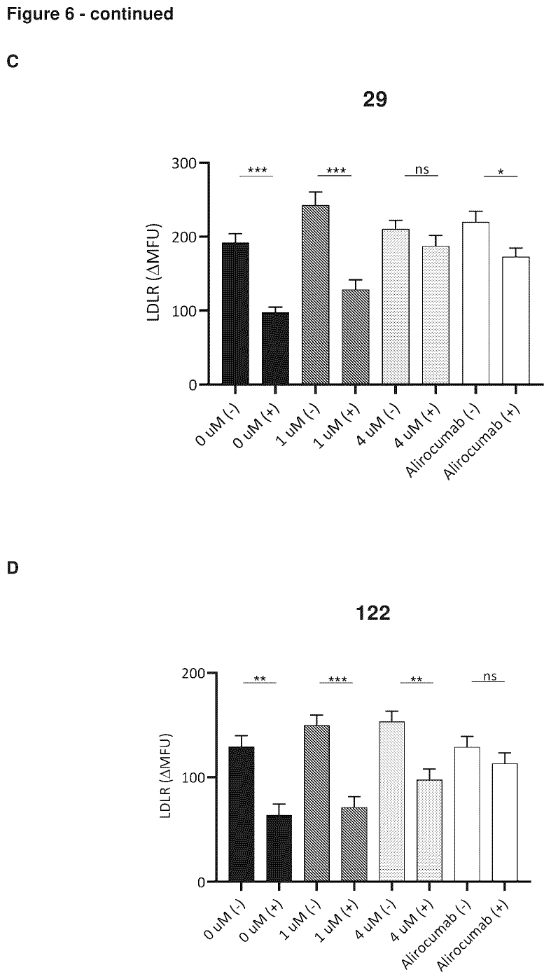

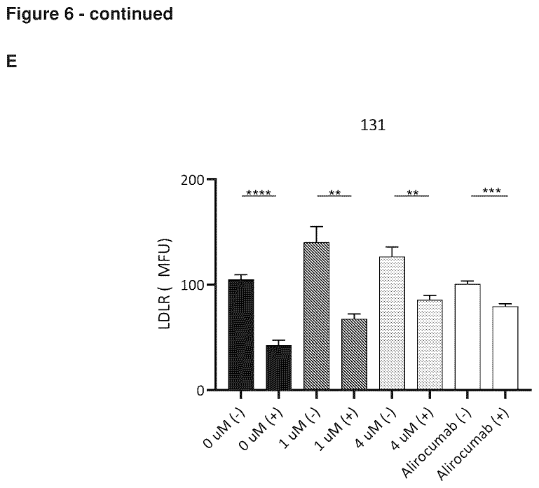

The results of the ex vivo LDLR surface expression on primary human lymphocytes are shown in FIGS. 6A-6E.

FIG. 6A summarises the percent decrease in LDLR expression levels on the surface of human primary lymphocytes upon the addition of recombinant PCSK9-D374Y to cells relative to non-PCSK9-D374Y-treated controls in the presence of either 0, 1 or 4 μM compound. Alirocumab was used as a positive control. The % decrease in LDLR levels diminishes as compound levels increase. This strongly indicates that each compound is protecting the LDLR from PCSK9-D374Y-directed degradation.

FIGS. 6B, C, D and E graphically illustrate the results summarized in FIG. 6A. Human PBMCs were cultured in the presence (+) or absence (−) of 600 ng/mL recombinant PCSK9-D374Y together with increasing doses of compound 3f^{#} (FIG. 6B), compound 29 (FIG. 6C), compound 122 (FIG. 6D) or compound 131 (FIG. 6E). Alirocumab was used as a positive control in each case. (Mean±SEM, P values: *≤0.05, **<0.01, ***<0.001). ^{#}Compound 3f—Evison, B. J., et al. Bioorg Med. Chem. 28, 115344 (2020):

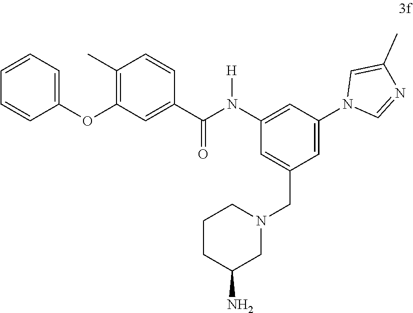 [SMILES: Cc1cn(-c2cc(CN3CCC[C@H](N)C3)cc(NC(=O)c3ccc(C)c(Oc4ccccc4)c3)c2)cn1]

### Example 11. Caco2 Assay

Caco-2 cells are a human colon epithelial cancer cell line used as a model of human intestinal absorption of drugs and other compounds. The compounds were assessed for their oral suitability in this assay, as compounds with good oral bioavailability likely evade efflux by pumps. The permeability coefficient (Papp) denotes the permeability of the drug through a monolayer of cells. Transport is assessed in both directions (apical to basolateral (A-B) and basolateral to apical (B-A)) across the cell monolayer to enable an efflux ratio to be determined which provides an indicator as to whether a compound undergoes active efflux.

Caco-2 cells were removed from an incubator and the cell monolayer was washed with HBSS buffer before the Transepithelial/transendothelial electrical resistance (TEER) was measured at room temperature. The transport experiment was started by adding 10 μM solution of test compounds in HBSS buffer containing luciferase yellow to the apical side. A sample from the apical chamber was taken at t=0 min. The volumes of the apical and basal sides were 400 μL and 800 μL respectively. The apical and basolateral plates were incubated at 37° C. for 90 min. After 90 min, the apical plate was separated from the basolateral plate and an aliquot from the basolateral side was transferred to an opaque plate for the determination of lucifer yellow concentration at t=90 min. The luciferase yellow concentration of each sample was measured by fluorimetry using excitation and emission wavelengths of 485 nm and 535 nm, respectively. Samples from the donor and receiver chambers were diluted with 0.4% DMSO in HBSS then mixed with ACN and either osalmid or imipramine, which were used as an IS in preparation for analysis by LC-MS/MS. Samples were then assayed for their levels of test compounds using an UPLC-MS/MS-29(API6500+) or an LC-MS/MS-014(API4000) instrument operating in a positive electrospray ionisation mode.

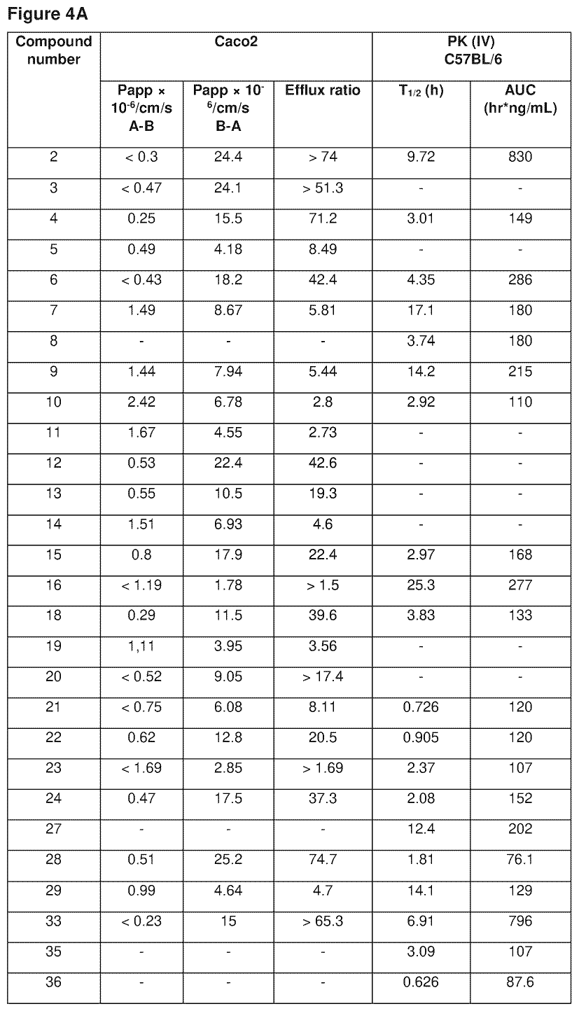

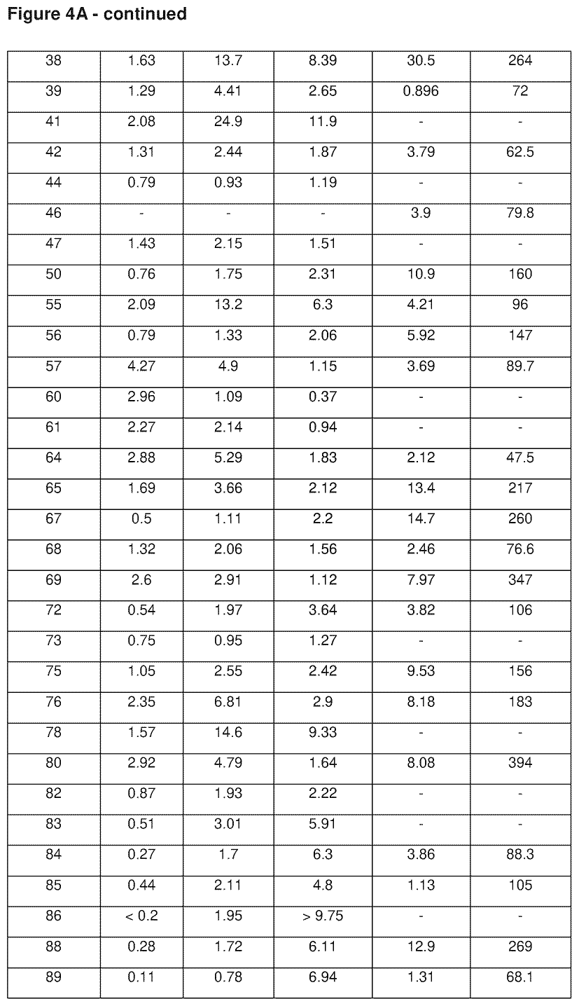

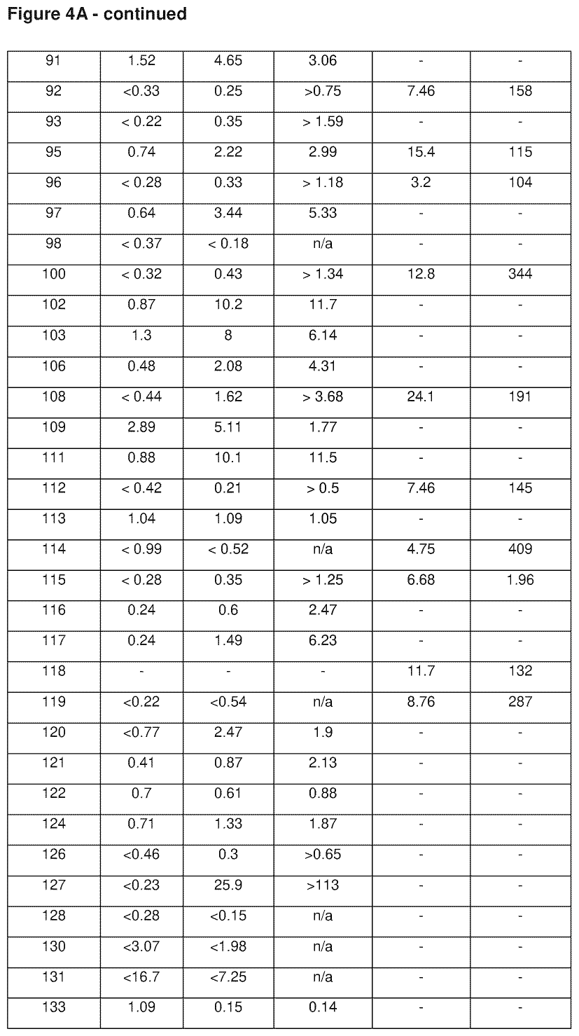

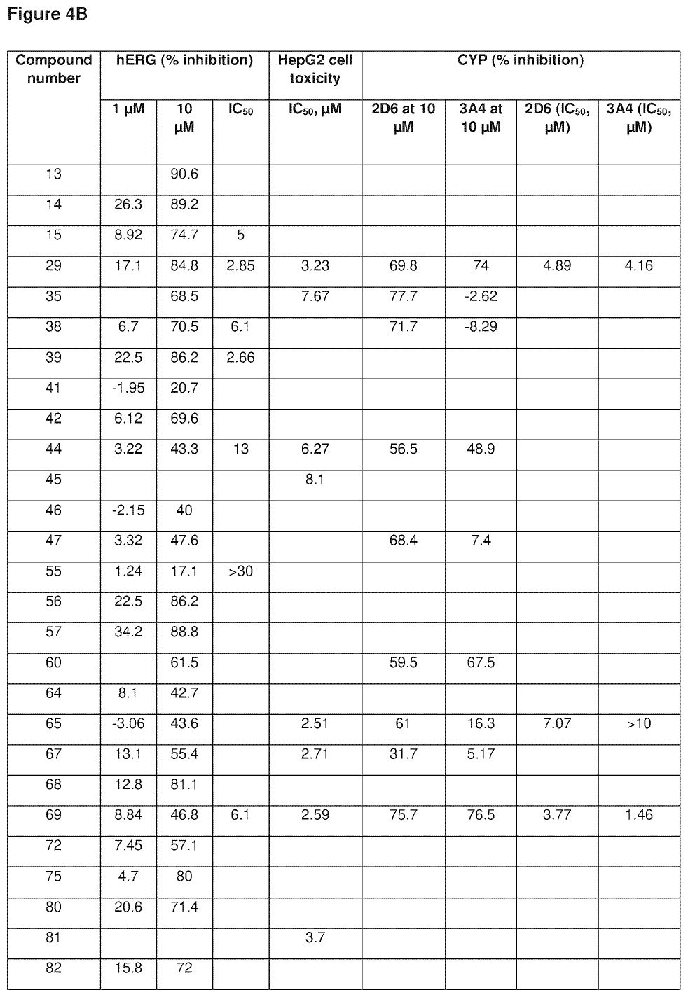

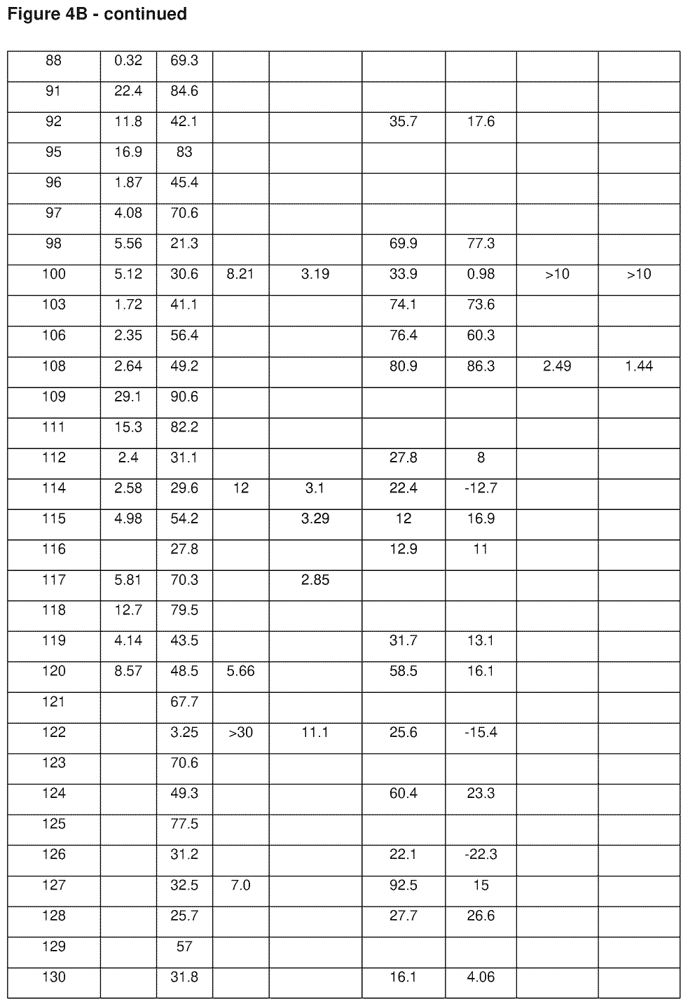

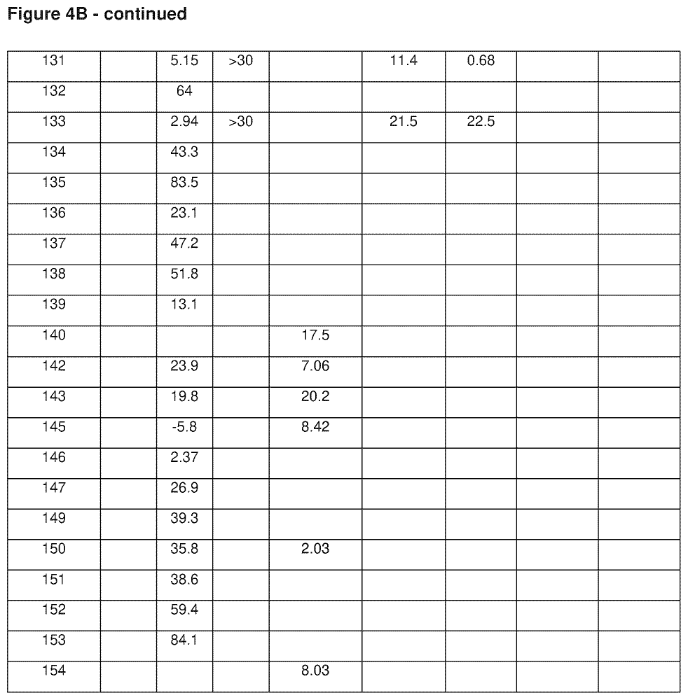

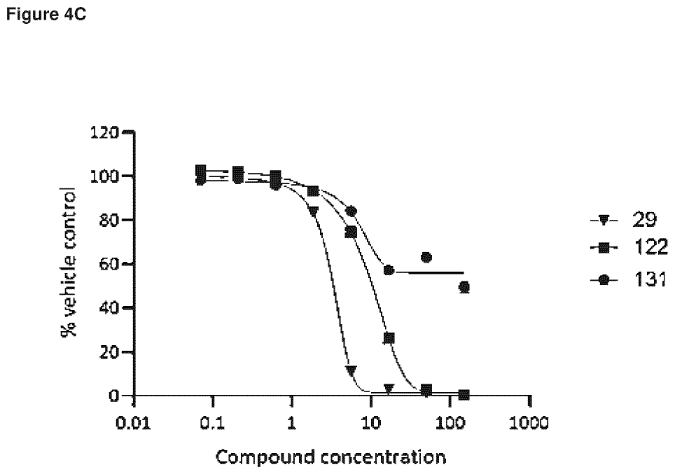

The results of the Caco2 assay are shown in FIG. 4A.

### Pharmacokinetics

### Example 12: Cassette Studies

Cassette dosing is a technique primarily used in drug discovery efforts in non-clinical studies to collect pharmacokinetic data from multiple drug candidates in a single experiment.

C57BL/6 mice (21-22 g, 6-8 weeks, male, purchased from Jihui Laboratory Animal Co. LTD) were allowed access to food and water ad libitum throughout the in-life phase of the study. Mice were dosed intravenously (IV) with a cassette of 0.4 mg/kg of test compounds in 5% DMAC, 5% Solutol HS15 and 90% saline. At designated time points, the animals were restrained manually, and ˜110 μL of blood/time point was collected into pre-cooled EDTA-K2 tubes via the facial vein. Blood samples were centrifuged at 4° C. (2000 g, 5 min) to obtain plasma within 15 min after sample collection. Plasma samples were stored at approximately −70° C. until analysis. Samples were assayed for their levels of test compounds by LC/MS/MS using a Triple Quadruple (6500+) system operating in a positive electrospray ionisation mode.

The results of the Cassette study are shown in FIG. 4A. The PK parameters of half-life (T_{1/2}) and area under the curve (AUC) of the compounds were assessed following a single intravenous (IV) injection (0.4 mg/kg) in mice (male, C57BL/6). These results for compounds 29, 122 and 131 are also summarised in Table 5 in Example 13 together with other PK data.

### Example 13: Dedicated Studies

A number of compounds—29, 122 and 131—indicated to have good bioavailability in the in vitro Caco2 study above (FIG. 4A) were further assessed in a dedicated in vivo pharmacokinetic study.

For dedicated pharmacokinetic analyses of compounds 29, 122 and 131, C57BL/6 mice (19-21 g, 10-12 weeks, female, purchased from Jihui Laboratory Animal Co. LTD) were given free access to food and water throughout the in-life phase of the study. Mice were orally dosed once with 50 mg/kg (10 mL/kg) of either compound 29, 122 and 131 with each compound formulated in 90% saline, 5% Solutol HS15 and 5% DMSO. At designated time points following dosing (t=0.083, 0.25, 0.5, 1, 2, 4, 8 and 24 h), the animals were restrained manually and ˜110 μL of blood/time point was collected into pre-cooled EDTA-K2 tubes via the facial vein. Blood samples were put on wet ice, then centrifuged at 2000 g for 5 min (4° C.) to obtain plasma within 15 min after sample collection. Plasma samples were subsequently stored at approximately −70° C. until analysis. Each sample was assayed for levels of 29, 122 and 131 by LC/MS/MS using a Triple Quadruple (6500+) system operating in a positive electrospray ionisation mode.

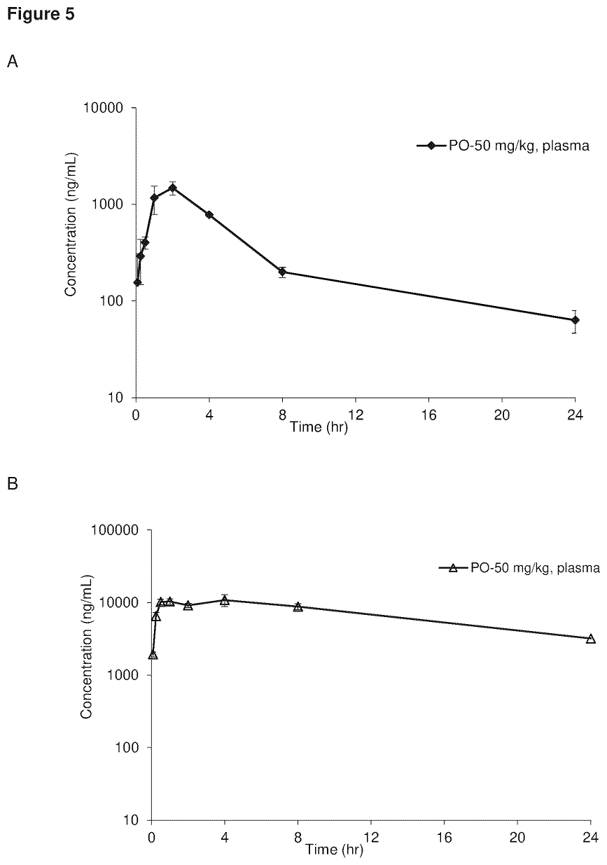

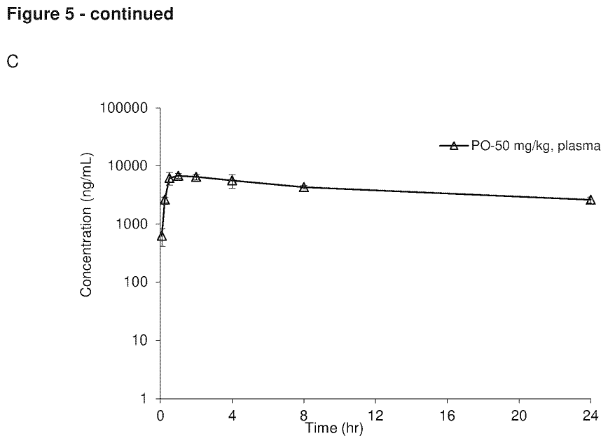

The mean plasma concentration-time profiles from dedicated pharmacokinetic analyses of compound 29, 122 and 131 are shown in FIG. 5A-C. Mice were orally dosed once with either 50 mg/kg of (A) compound 29, (B) compound 122 or (C) compound 131. The data demonstrate that compound 29 shows good bioavailability (F=40%) when administered orally while compound 122 (F=75%) and compound 131 (F=54%) exhibit very good and good oral bioavailability, respectively, under the same conditions.

### Example 14: Toxicity

Various additional ADMET properties of compounds of the invention were assessed.

HepG2 Cytotoxicity

HepG2 is a human hepatoma commonly used in drug metabolism and hepatotoxicity studies.

HepG2 cells were seeded at a density of 7000 or 8000 cells/well in a 96-well plate. Twenty to twenty four hours later, cells were treated with test compound for 48 hours in 3-fold serial dilutions with a top test concentration of 50 or 150 μM. After the incubation, CellTiter-Glo reagent was added directly to the wells. The plate was shaken for 2 or 5 mins on a plate shaker then incubated at room temperature for a further 10 mins. Subsequently, 100 μL of the above solution was transferred to a white flat bottom opaque 96 well plate (or opaque-walled clear bottom plates taped with a white bottom seal) and luminescence recorded on a plate reader.

hERG Assay

The hERG channel inhibition assay is a highly sensitive measurement which will identify compounds exhibiting cardiotoxicity related to hERG inhibition in vivo.

A CHO cell line stably transfected with hERG cDNA and expressing hERG channels was used. Cells used in the QPatch all met the following criteria: under microscopy examination, the majority of cells in suspension were single and isolated, their viability was greater than 95%, cell density ranged between 3-8×10⁶ cells/mL in the final suspension before applying to the QPatch stir chamber. Cells were used within 4 hours of harvesting. Whole-cell recordings were performed using automated QPatch (Sophion Bioscience, Denmark). The cells were voltage clamped at a holding potential of −80 mV. The hERG current was activated by depolarising at +20 mV for 5 seconds, after which the current was taken back to −50 mV for 2.5 seconds to remove the inactivation and observe the outward tail current. The maximum amount of tail current was used to determine hERG current amplitude.

CYP Inhibition Assay

In vitro CYP (cytochrome P450) enzyme inhibition studies predict a drug candidate's predisposition to inhibit cytochrome P450 enzymes.

The cytochrome P450 (CYP) inhibition assay was run against CYP2D6 and CYP3A4 isoforms using human liver microsomes. For CYP2D6, the probe substrates was Bufaralol (10 μM) and the positive control was Quinidine (2.5 μM). Midazolam (5 μM) was chosen as the probe substrate for CYP3A4 and Ketocanazole (2.5 μM) was used as the positive control inhibitor. To increase the throughput of the assay, one concentration (10 μM) was tested. For analysis via LC-MS, internal standards of 1-OH-Bufuralol-maleate-[D₉] for CYP2D6 and 1′-OH-Midazolam-13C₃ for CYP3A4 were used.

The ADMET properties determined by these assays are shown in FIG. 4B (together with other ADME and PK data in FIG. 4A). The cytotoxicity profile of compounds 29, 122 and 131 in HepG2 cells following 48 h of exposure to the compounds is illustrated in FIG. 4C. The results for compounds 29, 122 and 131 and are also summarized and compared in Table 5 below, together with compound 3f as a comparator/reference compound:

TABLE 5

| Parameter | | Compound 3f ** | Compound 29 | Compound 122 | Compound 131 |
|---|---|---|---|---|---|
| PCSK9-LDLR binding | | 939 | 320 | 279 | 547 |
| assay IC₅₀ (nM) | | | | | |
| Pharmacokinetic | Bioavailability | 0.5** | 40 | 75 | 54 |
| properties | (%) | | | | |
| | AUCₗₐₛₜ | 92.3** | 8,130 | 173,000 | 98,500 |
| | (h*ng/ml) | | | | |
| | T_{1/2} (h) | N/A | 6.29 | 11.3 | 19.0 |
| | C_{Max} (ng/ml) | 52.5** | 1,480 | 10,800 | 6,740 |
| | T_{Max} (h) | N/A | 2 | 4 | 1 |
| | V_{SS} (L/kg) | 3.87** | 14.7 | 1.29 | 3.25 |

| | | | | |
|---|---|---|---|---|
| CYP3A4 | 95.0 | 74.0 | −15.4 | 0.68 |
| (% inhibition at 10 μM) | | | | |
| CYP2D6 | 82.2 | 69.8 | 25.6 | 11.4 |
| (% inhibition at 10 μM) | | | | |
| hERG IC₅₀ (μM) | 19.0 | 2.85 | 35.7 | 94.2 |
| HepG2 cytotoxicity | | | | |
| IC₅₀ (μM) | 5.23 | 3.23 | 11.1 | 109 |

**Data from Evison, B.J., et al. Bioorg Med. Chem. 28, 115344 (2020). Compound 3f is also referred to as NYX-330 in some publications. 3f is illustrated above in Example 10.

### Example 15: In Vivo Efficacy Study: Compound 29 Monotherapy

Female APOE*3-Leiden.CETP mice (˜10-14 weeks old) were obtained from the SPF breeding stock at TNO-Metabolic Health Research (Leiden). These mice are a specialised mouse model which has demonstrated high predictability of human cholesterol metabolism and cardiovascular health outcomes.

Mice were housed in macrolon cages (maximum of 6 mice per cage) in clean-conventional animal rooms (relative humidity 50-60%, temperature ˜21° C., light cycle 7 am to 7 μm). Mice were fed a semi-synthetic western type diet with 15% saturated fat and 0.15% (w/w) cholesterol and were provided with heat-sterilized tap water and food ad libitum.

Forty female APOE*3-Leiden.CETP mice were allowed to acclimate to these conditions for a 3 week run-in period prior to the removal of 8 low-responder mice. The remaining 32 mice were divided into 4 study groups of 8 mice each, matched for age, body weight, and plasma cholesterol and triglycerides. In the final week of the run-in diet, all mice received vehicle solution (5% DMSO, 5% Solutol HS15, 90% saline) twice daily by oral gavage to accustom the animals to the procedure. After matching, mice received vehicle solution only (Group 1) or compound 29 in vehicle solution (Groups 2, 3 and 4) for the remainder of the study by twice daily administration using oral gavage in a volume of 5 mL/kg body weight. On days 0, 3, 7, 14, 21 and 28 of treatment, blood samples were taken after 4 h of fasting, from which EDTA plasma was collected and used for measurement of plasma total cholesterol. Lipoprotein profiles were determined on day 0, 14 and 28 in group pooled plasma samples using an equal volume of plasma per mouse. On day 28, directly after the last blood sampling point, mice were sacrificed via CO₂ asphyxiation, fasted. At sacrifice, EDTA-plasma samples were obtained via heart puncture.

EDTA plasma blood samples were obtained after 4 h fasting. Animals were placed under a red light lamp for at least 10 minutes. The tail was fixed and the mice were allowed to move freely during blood collection and were not fixed to avoid additional stress. An incision was made in the tail vein to collect tail blood using microvette tubes containing EDTA-dipotassium. Tubes containing blood were placed on ice immediately. Plasma was obtained after centrifugation (10 min at 4500×g) of the samples in a lab centrifuge at 4° C. Plasma was separated and stored in an Eppendorf vial and stored at −70° C. for further use.

Total plasma cholesterol and triglyceride levels were determined using a “Cholesterol CHOD-PAP” kit both from Roche.

Lipoprotein profiles were measured by FPLC analysis using an AKTA apparatus from GE Healthcare. Analyses were performed in samples pooled per group. Cholesterol levels were measured using a “Cholesterol CHOD-PAP” kit from Roche.

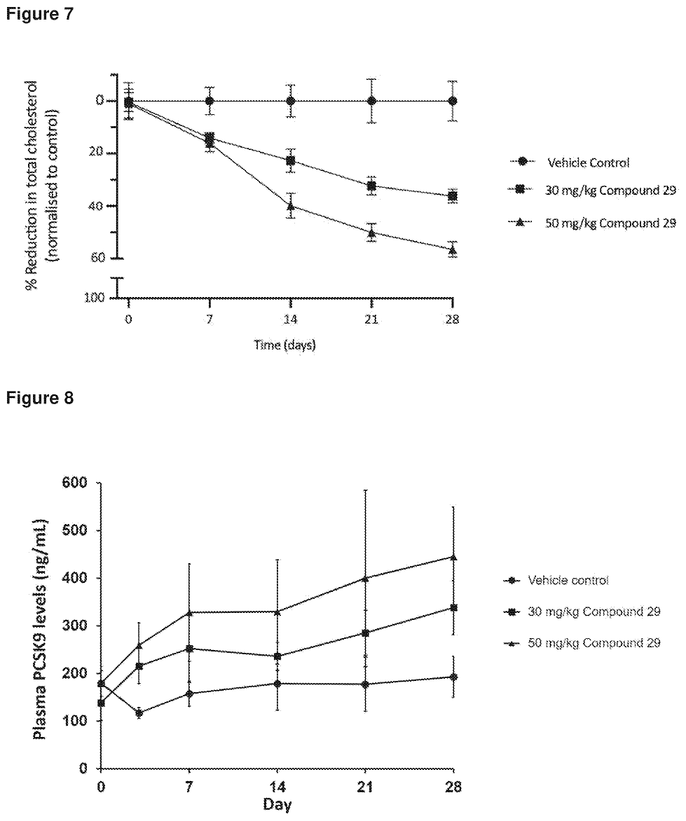

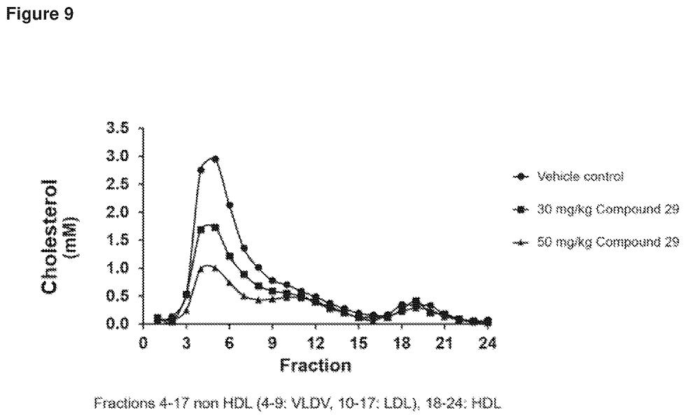

The results from the in-vivo efficacy studies are shown in FIGS. 7, 8 and 9. The results show that oral dosing of compound 29 at 30 mg/kg and 50 mg/kg were able to reduce total plasma cholesterol levels by more than 30% and 50%, compared to the vehicle control, respectively over a period of four weeks (FIG. 7 and Table 6 below).

TABLE 6

| | % difference in plasma total cholesterol versus vehicle control (P-value) | | | |
|---|---|---|---|---|
| Time (days) | 7 | 14 | 21 | 28 |
| 30 mg/kg compound 29 | −14% (0.05) | −23% (0.015) | −32% (0.002) | −36% (<0.001) |
| 50 mg/kg compound 29 | −16% (0.05) | −40% (<0.001) | −50% (<0.001) | −57% (<0.001) |

The results also show an increase in plasma PCSK9 levels over the same time-period (FIG. 8). An increase in PCSK9 plasma levels indicates that compound 29 is inhibiting the interaction of the protein with LDLR in vivo and, as such, PCSK9 cannot be taken up by the liver cells from the blood.

These results confirm the usefulness of the compound 29 to modulate PCSK9 levels, and reduce cholesterol concentrations in plasma following oral dosing of the compound.

### Example 16: In Vivo Efficacy Study: Compound 29 in Combination with Atorvastatin (Lipitor™)

Forty-four, 11-16 week old female APOE*3-Leiden.CETP transgenic mice were put on a Western-type diet (WTD) with 0.15% cholesterol and 15% saturated fat and sterilized tap water ad libitum. Throughout the study, mice were housed in macrolon cages (3-5 mice per cage) in clean-conventional animal rooms at TNO-Leiden; relative humidity 40-70%, temperature 20-24° C., light cycle 7 am to 7 μm. After 2 weeks of WTD feeding and one week prior to start of treatment, all animals were conditioned to a twice per day oral gavage procedure. After a 3-week run-in period on WTD, 12 low-responder mice were removed from the study. The remaining 32 mice were divided into 4 study groups of 8 mice each, matched for age, body weight, and plasma cholesterol and triglycerides after 4 hours of fasting. Treatment with the test compounds, atorvastatin and vehicle solution was started after matching.

Mice in Groups 1 and 3 were treated with vehicle solution alone (5% DMSO, 5% Solutol HS15, 90% saline). Mice in Groups 2 and 4 were treated with 50 mg/kg body weight of compound 29 in 5% DMSO, 5% Solutol HS15, 90% saline twice per day using a dose volume of 5 mL/kg body weight at around 08:00 and 16:00. Mice in Groups 3 and 4 were treated with atorvastatin as diet admix in a dose of 4.9 mg atorvastatin/kg body weight.

The body weight of each mouse and food intake (per cage) were measured on day 0 (prior to group matching) and on day 7, 14, 21, 28 and 35 of the study. Blood samples were collected after 4 hours of fasting on day 0, 7, 14, 21, 28 and 35 and processed to collect EDTA plasma as described above. Plasma total cholesterol (TC) and PCSK9 levels were measured on day 0, 7, 14, 21, 28 and 35. Plasma HDL-C was measured on day 0, 14, 28 and 35. Non-HDL-C levels were calculated from plasma TC and HDL-C levels. Lipoprotein profiles were determined in group-pooled samples on day 0, 14, 28 and 35. Group-pooled plasma samples contained an equal volume of plasma per mouse. On day 35, directly after the last fasted blood sampling point, mice were sacrificed via CO₂ asphyxiation. The livers were collected and snap-frozen in liquid N₂.

Mouse body weight and food intake were determined using a calibrated balance. Total plasma cholesterol levels were evaluated using a “Cholesterol CHOD-PAP”kit and from Roche. Plasma HDL-cholesterol was also determined using the “Cholesterol CHOD-PAP” kit from Roche after precipitation of apoB-containing lipids by using PEG-6000/glycine. Non-HDL cholesterol levels were calculated by subtracting HDL-Cholesterol levels from total cholesterol levels. Lipoprotein profiles were measured by FPLC analysis using an AKTA apparatus from GE Healthcare. Analyses were performed in samples pooled per group. Cholesterol levels were measured in the fractions using the “Cholesterol CHOD-PAP” from Roche. Plasma PCSK9 levels were measured using the Mouse Proprotein Convertase 9/PCSK9 Quantikine ELISA Kit (MPC900) from R&D Systems. Hepatic LDL receptor protein expression analysis was performed by Western blotting using liver tissue homogenates (Kühnast, S. et al. Alirocumab inhibits atherosclerosis, improves the plaque morphology, and enhances the effects of a statin. J. Lipid Res. 55, 2103 (2014)).

Results

The results from this experiment demonstrate at least an additive effect of combining compound 29 and atorvastatin (Lipitor).

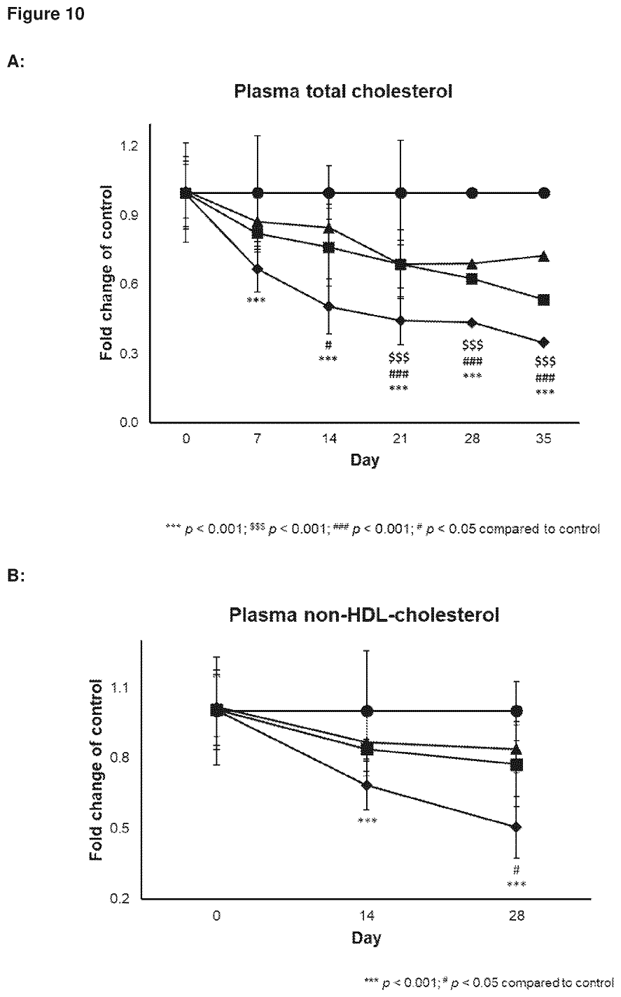

Plasma total cholesterol levels of female APOE*3-Leiden.CETP mice treated orally with either vehicle (solid circles), 4.9 mg/kg/day atorvastatin (solid triangles), 50 mg/kg compound 29 twice per day (solid squares) or a combination of 4.9 mg/kg/day and 50 mg/kg compound 29 twice per day (solid diamonds) are shown in FIG. 10A. Significant differences from vehicle-only control are indicated using $ for atorvastatin; # for compound 29 alone and * for combination of compound 29 and atorvastatin.

Plasma non-HDL-cholesterol levels of female APOE*3-Leiden.CETP mice treated orally with either vehicle (solid circles), 4.9 mg/kg/day atorvastatin (solid triangles), 50 mg/kg compound 29 twice per day (solid squares) or a combination of 4.9 mg/kg/day and 50 mg/kg compound 29 twice per day (solid diamonds) are shown in FIG. 10B. Significant differences from the vehicle-only control are indicated using # for compound 29 alone and * for the combination of compound 29 and atorvastatin.

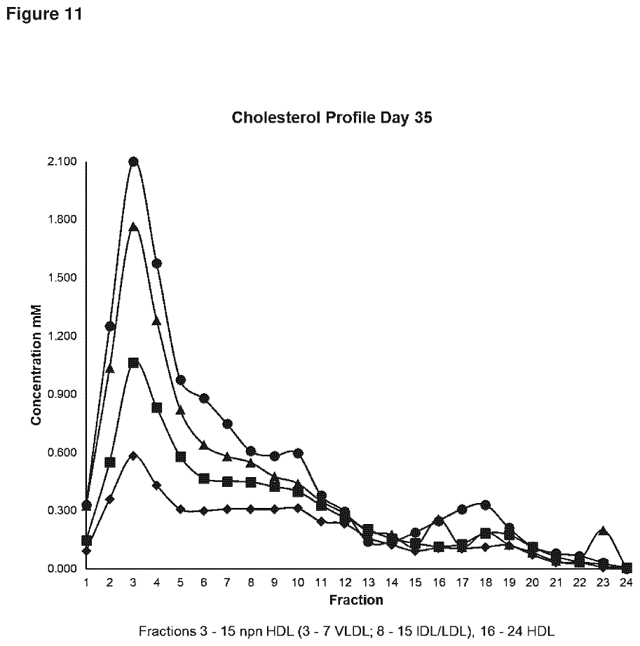

The fractionation of cholesterol levels according to lipoprotein type in pooled plasma samples of female APOE*3-Leiden.CETP mice following 35 days of treatment is shown in FIG. 11. The mice were treated orally with either vehicle (solid circles), 4.9 mg/kg/day atorvastatin (solid triangles), 50 mg/kg compound 29 twice per day (solid squares) or a combination of 4.9 mg/kg/day and 50 mg/kg compound 29 twice per day (solid diamonds).

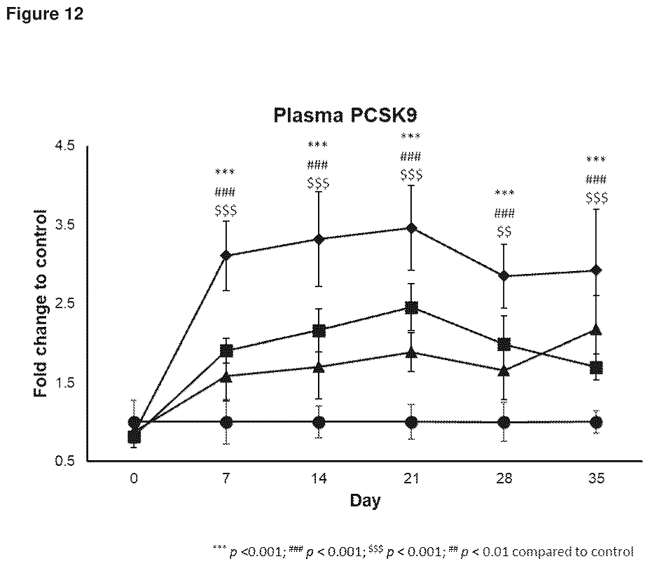

Plasma total PCSK9 levels of female APOE*3-Leiden.CETP mice treated orally with either vehicle (solid circles), 4.9 mg/kg/day atorvastatin (solid triangles), 50 mg/kg compound 29 twice per day (solid squares) or a combination of 4.9 mg/kg/day and 50 mg/kg compound 29 twice per day (solid diamonds) is shown in FIG. 12. Significant differences from the vehicle-only control are indicated using $ for atorvastatin; # for compound 29 alone and * for the combination of compound 29 and atorvastatin.

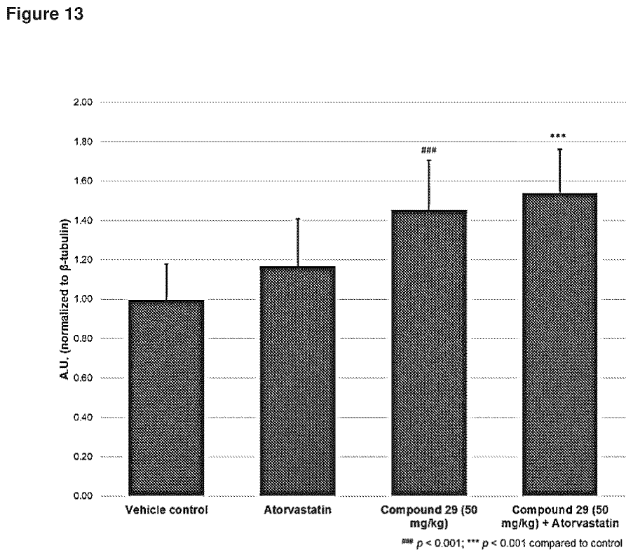

The hepatic levels of the LDL receptor in APOE*3-Leiden.CETP mice treated orally with either vehicle, 4.9 mg/kg/day atorvastatin, 50 mg/kg compound 29 twice per day or a combination of 4.9 mg/kg/day and 50 mg/kg compound 29 twice per day as indicated, is shown in FIG. 13. Significant differences relative to the vehicle-control are indicated using # for compound 29 alone and * for the combination of compound 29 and atorvastatin.

1. A compound according to Formula (I): 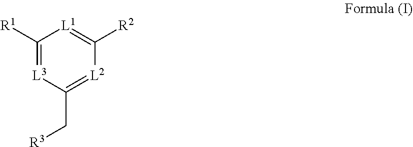
or a pharmaceutically acceptable salt, solvate, prodrug or polymorph thereof,
wherein
L¹, L² and L³ are independently CH or N;
R¹ is —NHCOR¹¹ or 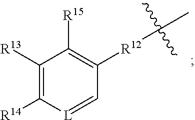
wherein L is CH or N;
R² is 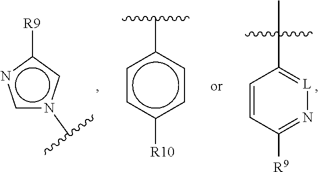
wherein L is CH or N;
R³ is —OCH₂CH₂NH₂, 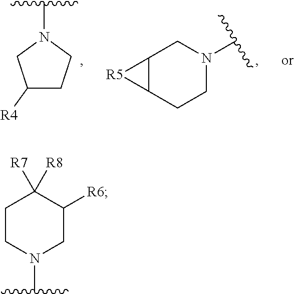
R⁴ is —NH₂ or —NHCH₃;
R⁵ is —NHCH₂CH₂O— or —OCH₂CH₂NH—;
R⁶ is —NH₂, —OH, —NHCOR¹⁶, or —NHSO₂CH₂CH₃;
R⁷ is —H, —OH, or —F;
R⁸ is —H or —F;
R⁹ is —CH₃ or —CF₃;
R¹⁰ is —H or —CH₃;
R¹¹ is 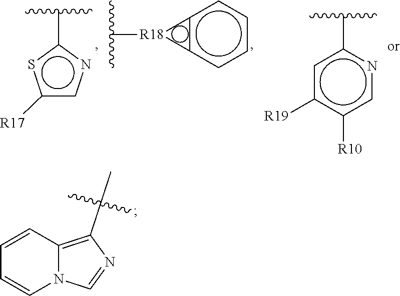
R¹² is —*CH₂NH—, —*CONH—, —*SO₂NH—, 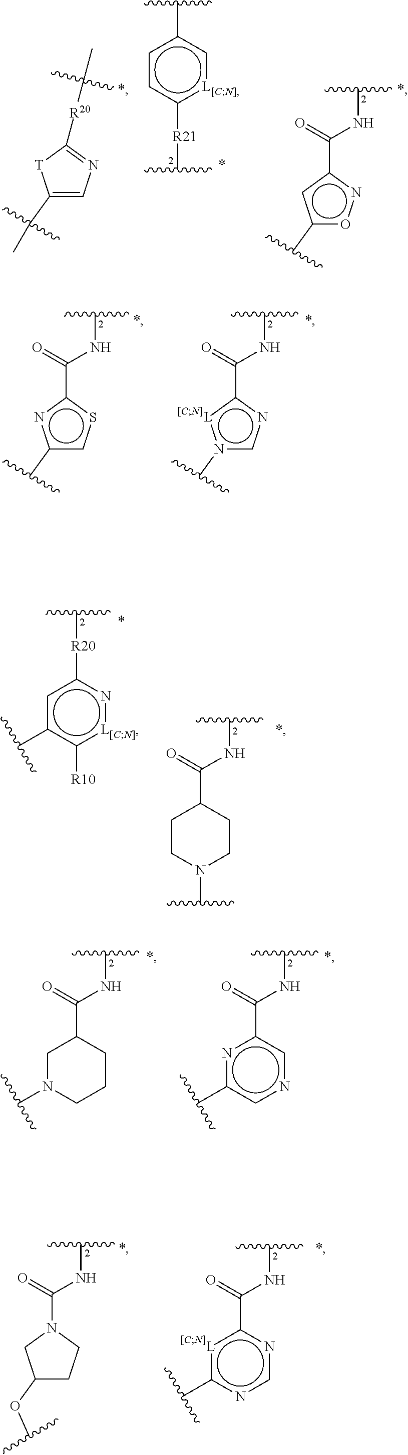 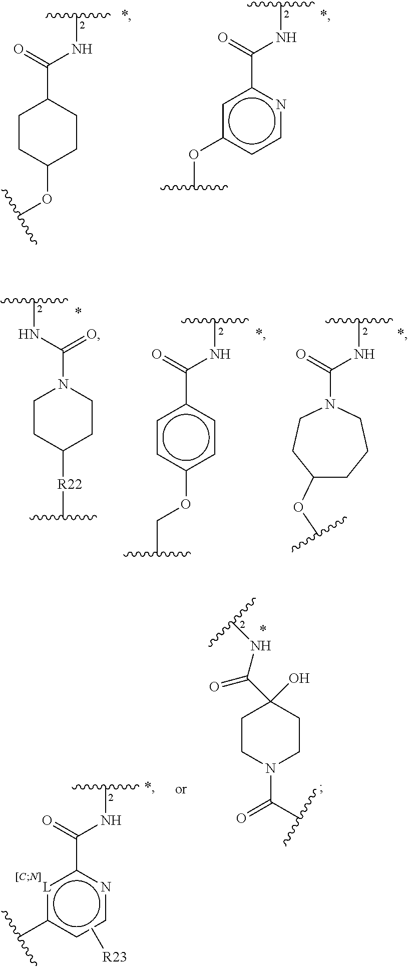
wherein the * indicates the attachment of R¹² to the carbon of the central ring of formula (I) and the other attachment point signifies the attachment of R¹² to the phenyl ring of R¹;
wherein T is O or S and L is CH or N;
R¹³ is —H, —CH₃, —F, —OCH₃, —CHCH₃OH, —SCH₃O₂, 2-methoxyethoxy, phenyl, or oxolan-3-yloxy;
R¹⁴ is —H, —CH₃, —OH, —F, —Cl, —OCH₃, cyclopropoxy, —OCF₃, —OCH(R¹⁰)CH₂R²⁷ wherein R¹⁰ is H or CH₃, or 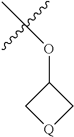
Q wherein Q is CH₂ or O;
R¹⁵ is —H or —CHF₂;
R¹⁶ is —CH₂CH₃, 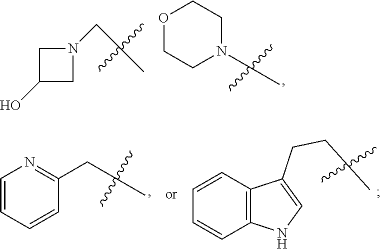
R¹⁷ is 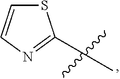 [SMILES: *c1nccs1]
cyclohexyl, 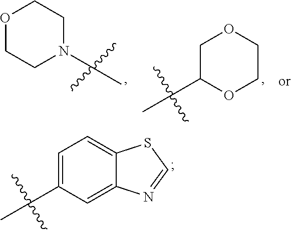 [SMILES: *N1CCOCC1 ; *C1COCCO1 ; *c1ccc2scnc2c1]
R¹⁸ is 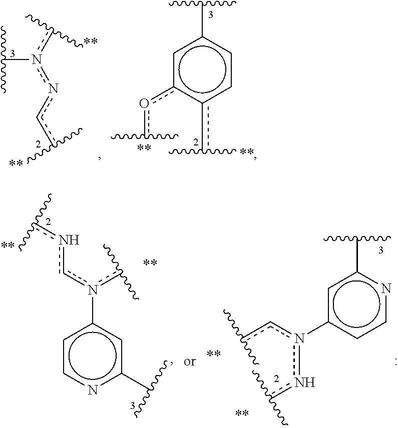
wherein the ** indicates the attachment points of R¹⁸ which form the fused ring and the remaining attachment point indicates the attachment to the carbonyl carbon of R¹;
R¹⁹ is —H, —(CH₃)₃, 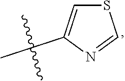 [SMILES: *c1cscn1]
cyclohexyl, 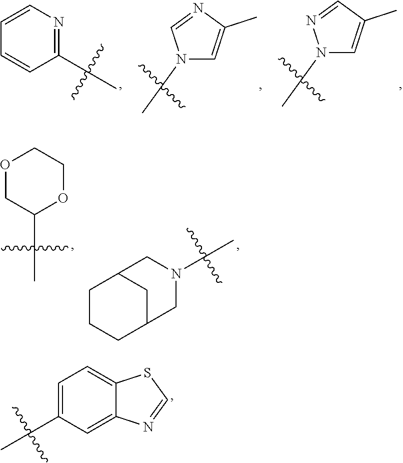
2,3,4,5,6-deuterated phenyl, 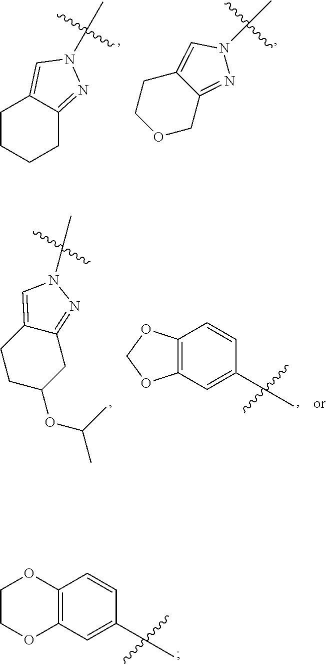 [SMILES: *n1cc2c(n1)CCCC2 ; *n1cc2c(n1)COCC2 ; *n1cc2c(n1)CC(OC(C)C)CC2 ; *c1ccc2c(c1)OCO2 ; *c1ccc2c(c1)OCCO2]
R²⁰ is —CH₂NH*— or —CONH*—; wherein the * indicates the attachment of R²⁰ to the carbon of the central ring of Formula (I);
R²¹ is —*CONH—, 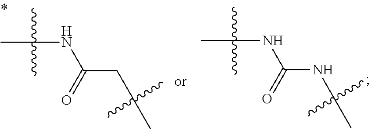 [SMILES: *CC(=O)N* ; *NC(=O)N*]
wherein the * indicates the preferred attachment of R²¹ to the carbon of the central ring of Formula (I);
R²² is —O— or —*OCH₂—; wherein the * indicates the attachment of R²² to the phenyl ring of R¹²;
R²³ is —CH₃ or —OCH₃;
R²⁷ is —H, —OH, —OCH₃, and —OCH(R¹⁰)CH₂R³⁰; wherein R¹⁰ is —H or —CH₃; and
R³⁰ is selected from the group consisting of —H and —OCH₂CH₂OCH₂CH₂OCH₂CH₂OCH₂CH₂OCH₂CH₂OR¹⁰, wherein R¹⁰ is H or CH₃.

2. A compound or a pharmaceutically acceptable salt, solvate, prodrug or polymorph thereof according to claim 1 wherein: R¹² is —CONH*—, 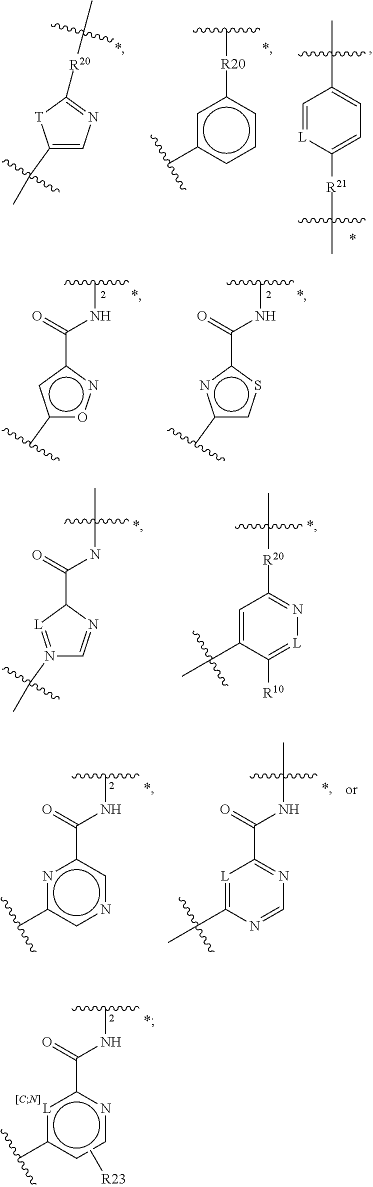
wherein T is O or S, wherein L is CH or N, and wherein the * indicates the attachment of R¹² to the carbon on the central ring of Formula (I); R¹³ is —H, —CH₃, —OCH₃, —CHCH₃OH, —SCH₃O₂, or 2-methoxyethoxy;
R¹⁴ is —H, —CH₃, —OH, —F, —OCH₃, cyclopropoxy, —OCH₂CH₂R²⁷, —OCH(CH₃)CH₂R²⁷ 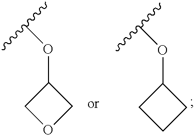 [SMILES: *OC1COC1 ; *OC1CCC1]
R¹⁸ is 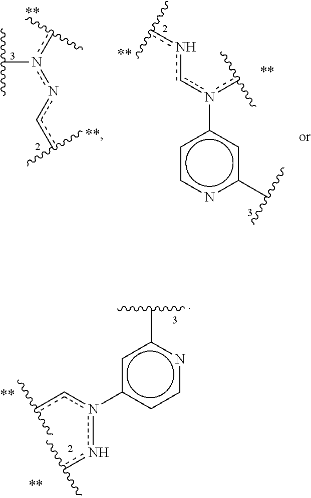
R²³ is —CH₃ or —OCH₃.

3. A compound or a pharmaceutically acceptable salt, solvate, prodrug or polymorph or deuterated analogue thereof according to claim 1, wherein the compound is a compound of formula (III) 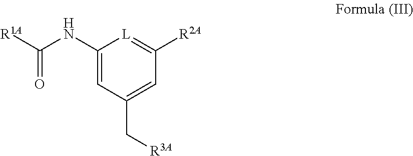
wherein L is CH or N;
wherein R^{1A} is 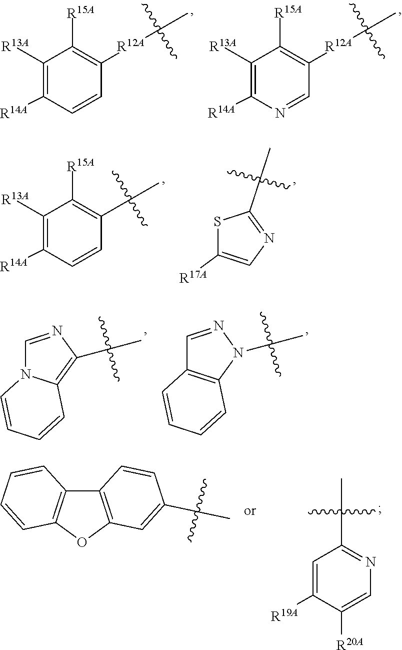
wherein R^{12A} is selected from 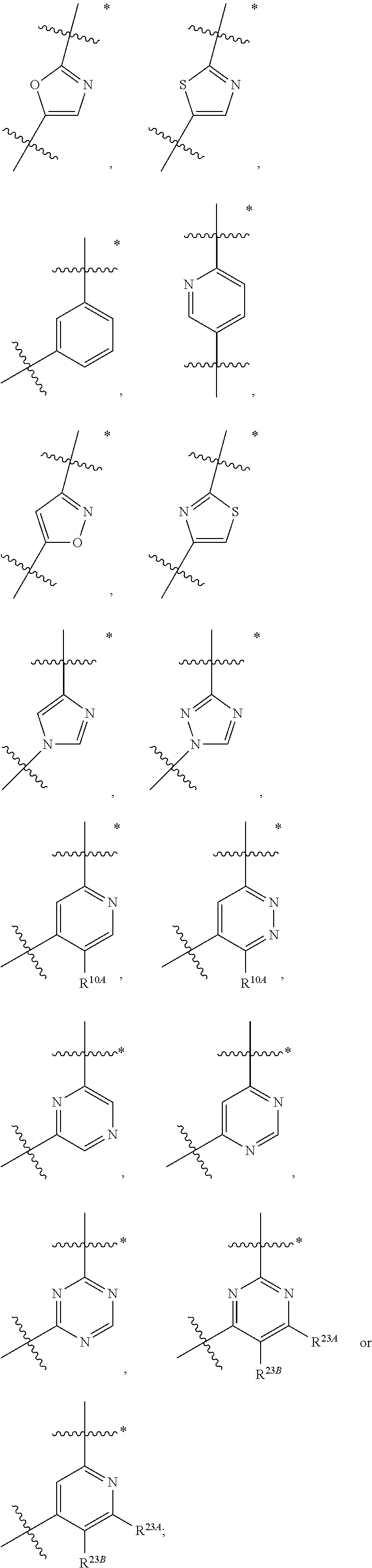
wherein the * signifies the attachment of R^{12A} to the carbonyl carbon of Formula (III) and the other attachment point signifies the attachment of R^{12A} to the phenyl ring of R^{1A};
R^{2A} and R^{3A} are as defined for R² and R³ in claim 1;
R^{10A} is —H or —CH₃;
R^{23A} and R^{23B} are independently —H, —CH₃ or —OCH₃;
R^{13A} is as defined for R¹³ in claim 1;
R^{14A} is —H, —CH₃, —OH, —F, —OCH₃, cyclopropoxy, —OCH₂CH₂OCH₃, —OCH₂CH₂OH, —OCH(CH₃)₂, —OCH₂CH₂OCH(CH₃)₂, —O(CH₂CH₂O)₇CH₃, —O(CH₂CH₂O)₇H, 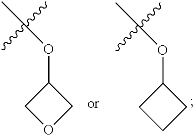 [SMILES: *OC1COC1 ; *OC1CCC1]
R^{15A} is as defined for R¹⁵ in claim 1;
R^{17A} is as defined for R¹⁷ in claim 1;
R^{19A} is as defined for R¹⁹ in claim 1; and
R^{20A} is —H or —CH₃.

4. A compound or a pharmaceutically acceptable salt, solvate, prodrug or polymorph or deuterated analogue thereof according to claim 1, wherein the compound is a compound of formula (IIIa) or formula (IIIb) 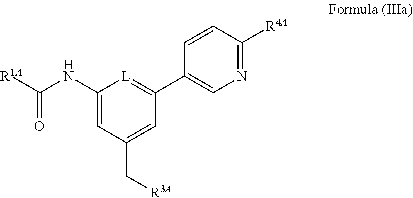 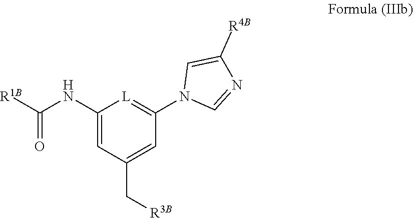
wherein L is CH or N;
R^{4A} and R^{4B} are independently CH₃ or CF₃;
R^{1A} and R^{1B} are independently as defined for R^{1A} in claim 3; and
R^{3A} and R^{3B} are independently as defined for R^{3A} in claim 3.

5. A compound or a pharmaceutically acceptable salt, solvate, prodrug or polymorph or deuterated analogue thereof according to claim 1, wherein the compound is a compound of formula (IVa) or formula (IVb) 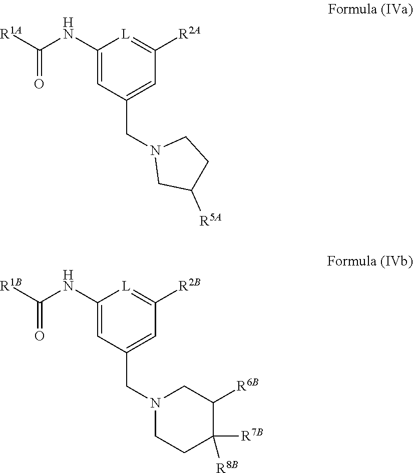
wherein L is CH or N;
R^{5A} is —NH₂ or —NHCH₃;
R^{6B} is defined as for R⁶ in claim 1;
R^{7B} is defined as for R⁷ is claim 1;
R^{8B} is defined as for R⁷ is claim 1;
R^{1A} and R^{1B} are independently as defined for R^{1A} in claim 3; and
R^{2A} and R^{2B} are independently as defined for R^{2A} in claim 3.

6. A compound or a pharmaceutically acceptable salt, solvate, prodrug or polymorph or deuterated analogue thereof wherein the compound is a compound of Formula (V) 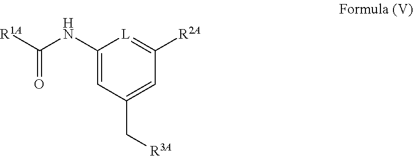
wherein L is CH or N;
R^{1A} is 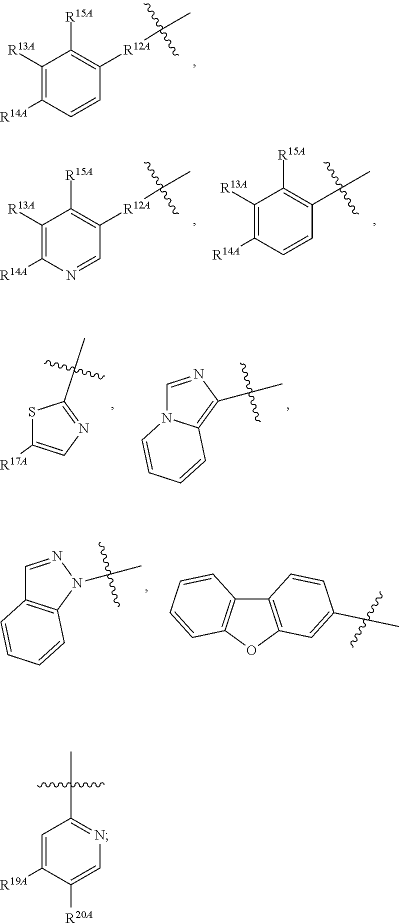
R^{12A} is selected from 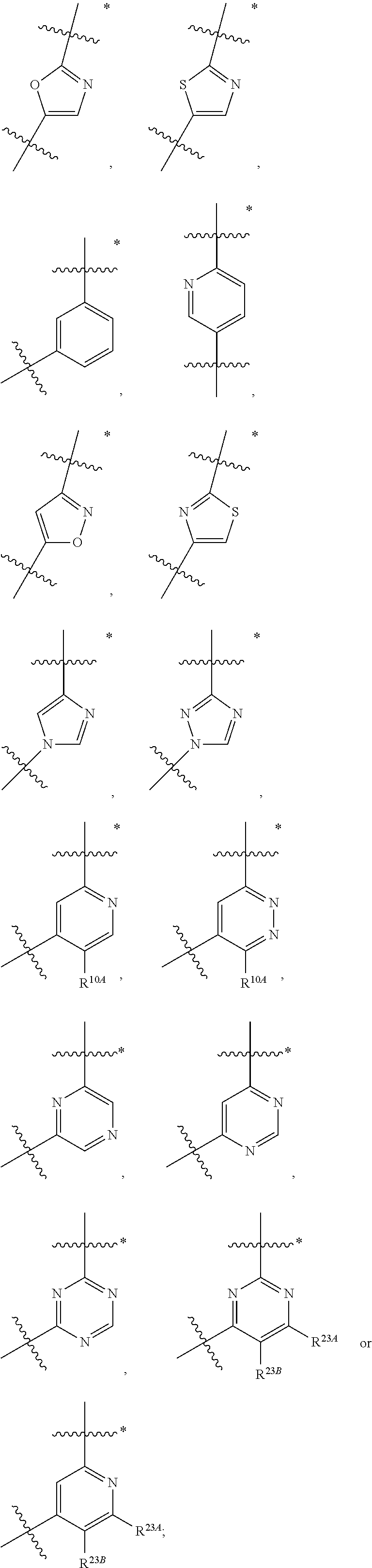
wherein the * signifies the attachment of R^{12A} to the carbonyl carbon of Formula (V) and the other attachment point signifies the attachment of R^{12A} to the phenyl ring of R^{1A};
R^{10A} is —H, —CH₃, C₂-C₆alkyl or C₁-C₆ fluoroalkyl;
R^{23A} and R^{23B} are independently —H, —CH₃, —OCH₃, C₂-C₆alkyl, or C₂-C₆alkoxy;
R^{13A} is —H, —CH₃, —F, —OCH₃, —CH₂CH₃OH, —SCH₃O₂, 2-methoxyethoxy, phenyl, or oxolan-3-yloxy;
R^{14A} is —H, —CH₃, C₂-C₆ alkyl, —OH, —F, —Cl, —I, —Br, —OCH₃, —OC₂-C₆alkyl, cyclopropoxy, —OCH₂CH₂OCH₃, —OCH₂CH₂OH, —OCH(CH₃)₂, —OCH₂CH₂OCH(CH₃)₂, —O(CH₂CH₂O)₇CH₃, —O(CH₂CH₂O)₇H, —O(CH₂CH₂O)ₙCH₃, or —O(CH₂CH₃O)ₙH, wherein n is 2, 3, 4, 5, or 6. 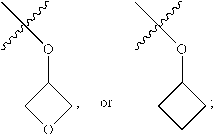 [SMILES: *OC1COC1 ; *OC1CCC1]
R^{15A} is —H, —CHF₂, —CH₂F, —CF₃, —C₁-C₆alkyl, or C₂-C₆ fluoroalkyl;
R^{17A} is 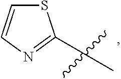 [SMILES: *c1nccs1]
cyclohexyl, C₃-C₅ cycloalkyl, 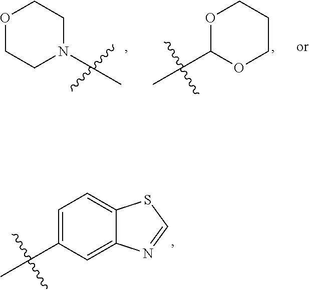 [SMILES: *N1CCOCC1 ; *C1OCCCO1 ; *c1ccc2scnc2c1]
R^{19A} is —H, —C(CH₃)₃, -n-butyl, isobutyl, C₁-C₃alkyl, C₅-C₆alkyl, C₁-C₆ fluoroalkyl, 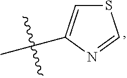 [SMILES: *c1cscn1]
cyclohexyl, C₃-C₅ cycloalkyl, 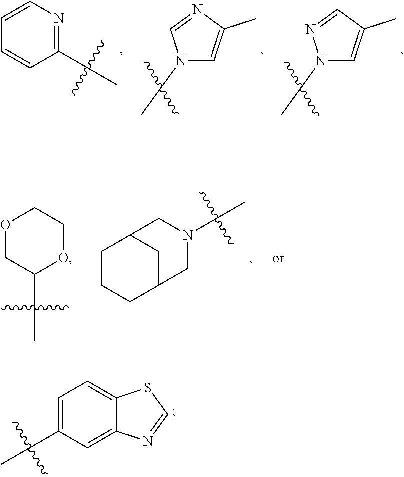
R^{20A} is —H, —CH₃, C₂-C₆ alkyl, C₁-C₆ fluoroalkyl, substituted C₁ alkyl, or substituted C₂-C₆ alkyl;
R^{2A} is selected from the group consisting of 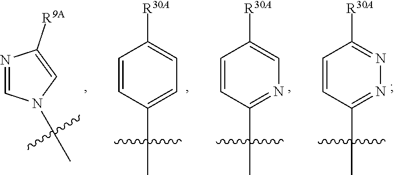
wherein R^{9A} is —CH₃, —CF₃, C₂-C₆alkyl, C₂-C₆ fluoroalkyl, —CH₂F, —CHF₂;
R^{30A} is H, —F, —CH₃, —C₂-C₆alkyl or C₁-C₆ fluoroalkyl;
R^{3A} is selected from the group consisting of —OCH₂CH₂NH₂, 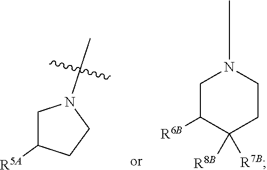
wherein R^{5A} is —NH₂, —NHCH₃ or —C₂-C₆alkylamino;
R^{6B} is —NH₂, —NH₂CH₃, C₂-C₆alkylamino, —OH, —NHCOR^{16B}, or —NHSO₂CH₂CH₃;
R^{16B} is —CH₂CH₃, —CH₃, —C₃-C₆alkyl, 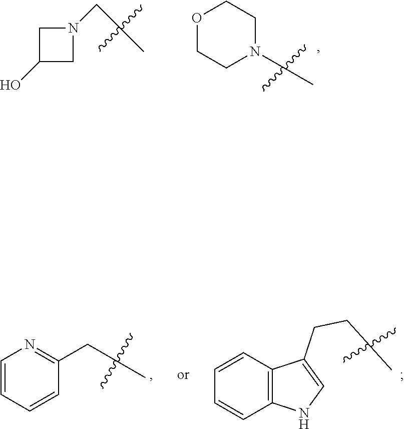 [SMILES: *CN1CC(O)C1 ; *N1CCOCC1 ; *Cc1ccccn1 ; *CCc1c[nH]c2ccccc12]
R^{7B} is —H, —OH, —F, —Cl, —Br, —I or —C₁-C₃alkyl;
R^{8B} is —H, —F, —Cl, —Br, —I or —C₁-C₃alkyl;

7. A compound or a pharmaceutically acceptable salt, solvate, prodrug or polymorph or deuterated analogue thereof according to claim 6, wherein the compound is a compound of formula (Va) or formula (Vb): 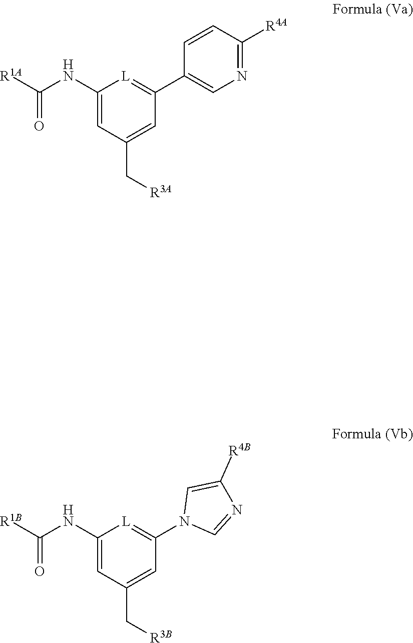
wherein
L is CH or N;
R^{4A} and R^{4B} are independently —CH₃, —CF₃, —C₂-C₆ alkyl or —C₂-C₆ fluoroalkyl;
R^{1A} and R^{1B} are independently as defined for R^{1A} in claim 6; and
R^{3A} and R^{3B} are independently as defined for R^{3A} in claim 6.

8. A compound or a pharmaceutically acceptable salt, solvate, prodrug or polymorph or deuterated analogue thereof according to claim 6, wherein the compound is a compound of formula (VIa) or formula (VIb): 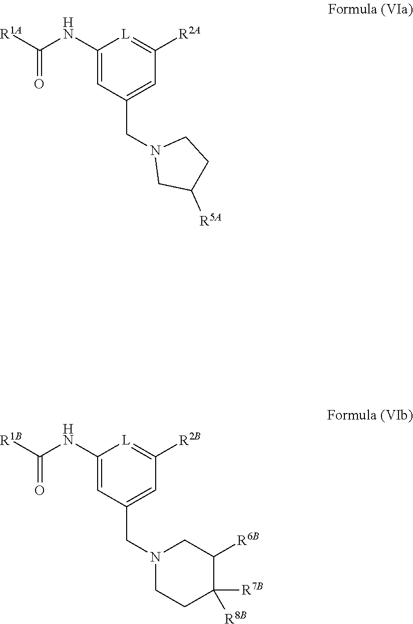
wherein
L is CH or N;
R^{5A} is —NH₂, —NHCH₃ or —C₂-C₆alkylamino;
R^{6B} is —NH₂, —NH₂CH₃, —C₂-C₆alkylamino;
R^{7B} is —H, —OH, —F, —Cl, —Br, or —I;
R^{8B} is —H, —F, —Cl, —Br, or —I;
R^{1A} and R^{1B} are independently as defined for R^{1A} in claim 6; and
R^{3A} and R^{3B} are independently as defined for R^{3A} in claim 6.

9. A compound selected from the group consisting of: 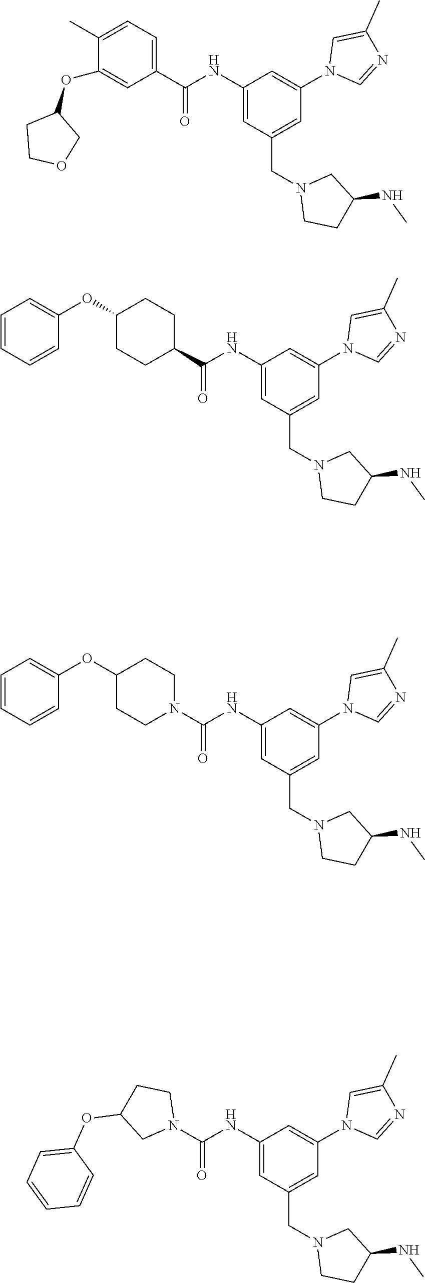 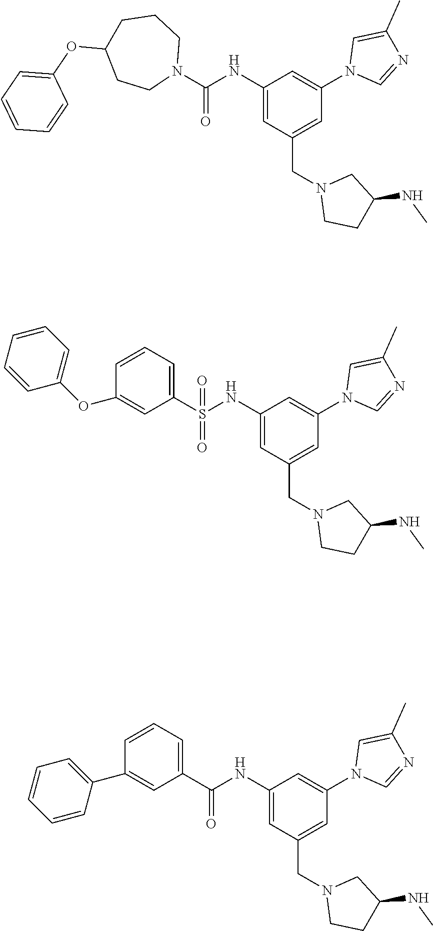 [SMILES: CN[C@H]1CCN(Cc2cc(NC(=O)N3CCCC(Oc4ccccc4)CC3)cc(-n3cnc(C)c3)c2)C1 ; CN[C@H]1CCN(Cc2cc(NS(=O)(=O)c3cccc(Oc4ccccc4)c3)cc(-n3cnc(C)c3)c2)C1 ; CN[C@H]1CCN(Cc2cc(NC(=O)c3cccc(-c4ccccc4)c3)cc(-n3cnc(C)c3)c2)C1] 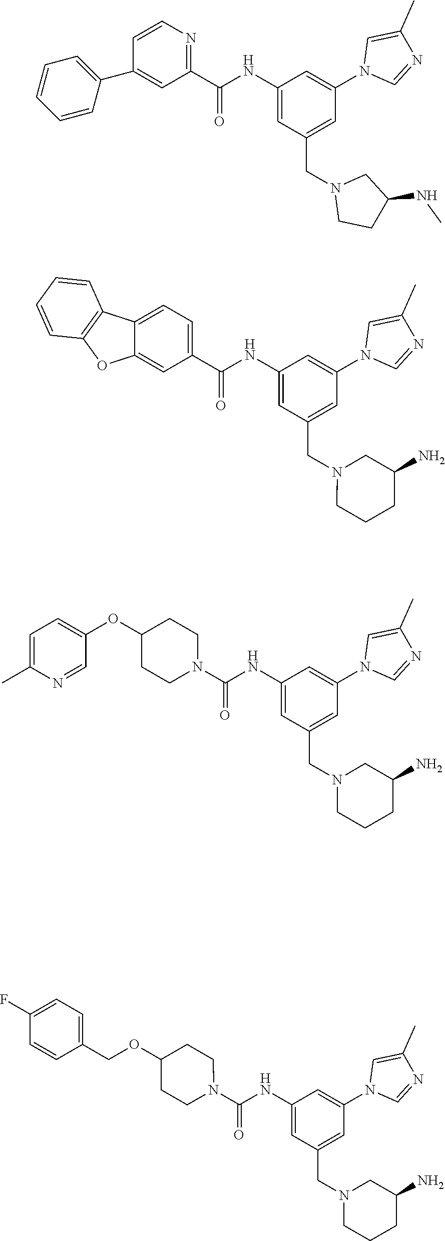 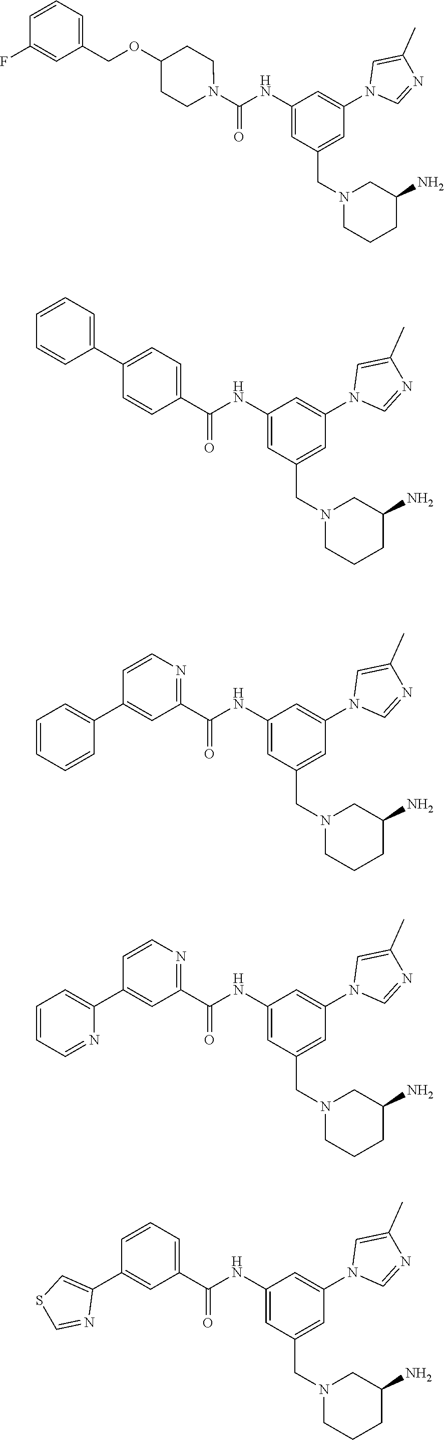 [SMILES: Cc1cn(-c2cc(CN3CCC[C@H](N)C3)cc(NC(=O)N3CCC(OCc4cccc(F)c4)CC3)c2)cn1 ; Cc1cn(-c2cc(CN3CCC[C@H](N)C3)cc(NC(=O)c3ccc(-c4ccccc4)cc3)c2)cn1 ; Cc1cn(-c2cc(CN3CCC[C@H](N)C3)cc(NC(=O)c3cc(-c4ccccc4)ccn3)c2)cn1 ; Cc1cn(-c2cc(CN3CCC[C@H](N)C3)cc(NC(=O)c3cc(-c4ccccn4)ccn3)c2)cn1 ; Cc1cn(-c2cc(CN3CCC[C@H](N)C3)cc(NC(=O)c3cccc(-c4cscn4)c3)c2)cn1] 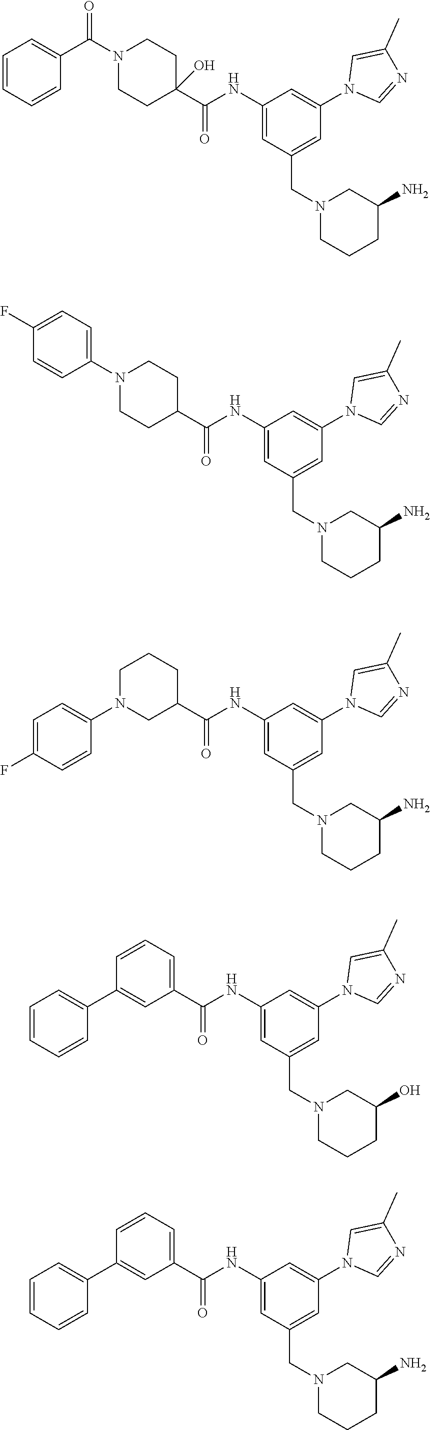 [SMILES: Cc1cn(-c2cc(CN3CCC[C@H](N)C3)cc(NC(=O)C3(O)CCN(C(=O)c4ccccc4)CC3)c2)cn1 ; Cc1cn(-c2cc(CN3CCC[C@H](N)C3)cc(NC(=O)C3CCN(c4ccc(F)cc4)CC3)c2)cn1 ; Cc1cn(-c2cc(CN3CCC[C@H](N)C3)cc(NC(=O)C3CCCN(c4ccc(F)cc4)C3)c2)cn1 ; Cc1cn(-c2cc(CN3CCC[C@H](O)C3)cc(NC(=O)c3cccc(-c4ccccc4)c3)c2)cn1 ; Cc1cn(-c2cc(CN3CCC[C@H](N)C3)cc(NC(=O)c3cccc(-c4ccccc4)c3)c2)cn1] 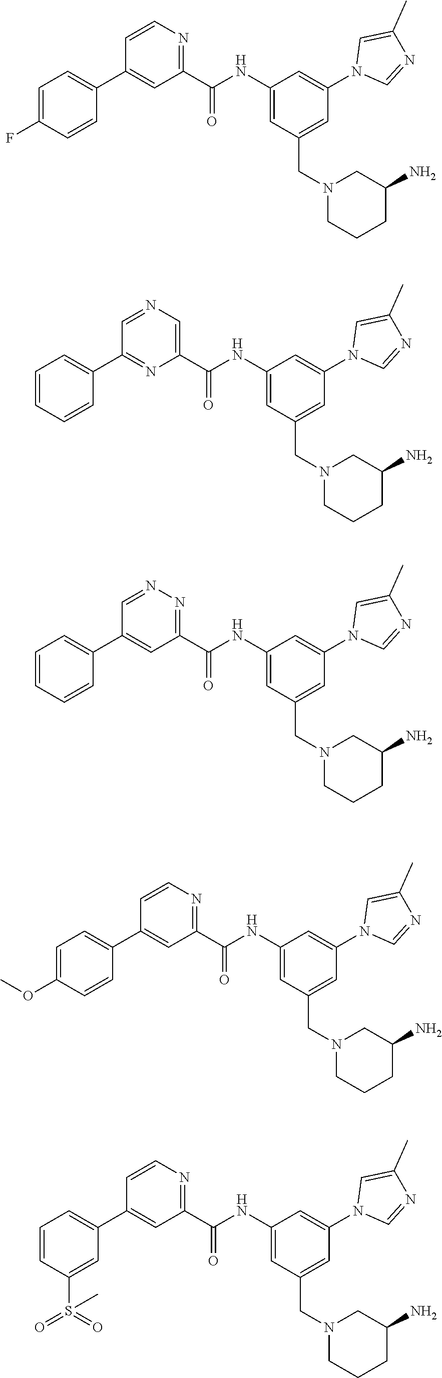 [SMILES: Cc1cn(-c2cc(CN3CCC[C@H](N)C3)cc(NC(=O)c3cc(-c4ccc(F)cc4)ccn3)c2)cn1 ; Cc1cn(-c2cc(CN3CCC[C@H](N)C3)cc(NC(=O)c3cncc(-c4ccccc4)n3)c2)cn1 ; Cc1cn(-c2cc(CN3CCC[C@H](N)C3)cc(NC(=O)c3cc(-c4ccccc4)cnn3)c2)cn1 ; COc1ccc(-c2ccnc(C(=O)Nc3cc(CN4CCC[C@H](N)C4)cc(-n4cnc(C)c4)c3)c2)cc1 ; Cc1cn(-c2cc(CN3CCC[C@H](N)C3)cc(NC(=O)c3cc(-c4cccc(S(C)(=O)=O)c4)ccn3)c2)cn1] 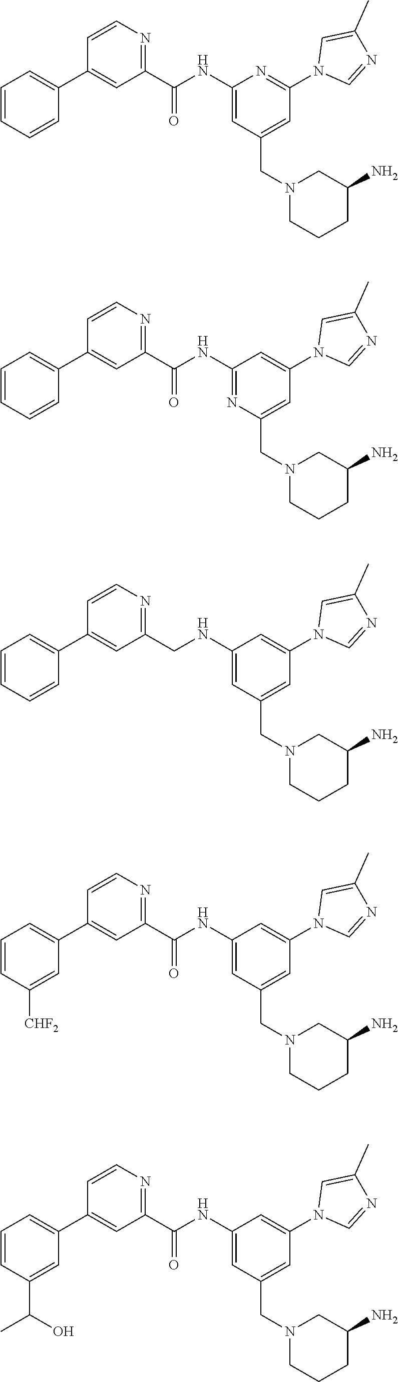 [SMILES: Cc1cn(-c2cc(CN3CCC[C@H](N)C3)cc(NC(=O)c3cc(-c4ccccc4)ccn3)n2)cn1 ; Cc1cn(-c2cc(CN3CCC[C@H](N)C3)nc(NC(=O)c3cc(-c4ccccc4)ccn3)c2)cn1 ; Cc1cn(-c2cc(CN3CCC[C@H](N)C3)cc(NCc3cc(-c4ccccc4)ccn3)c2)cn1 ; Cc1cn(-c2cc(CN3CCC[C@H](N)C3)cc(NC(=O)c3cc(-c4cccc(C(F)F)c4)ccn3)c2)cn1 ; Cc1cn(-c2cc(CN3CCC[C@H](N)C3)cc(NC(=O)c3cc(-c4cccc(C(C)O)c4)ccn3)c2)cn1] 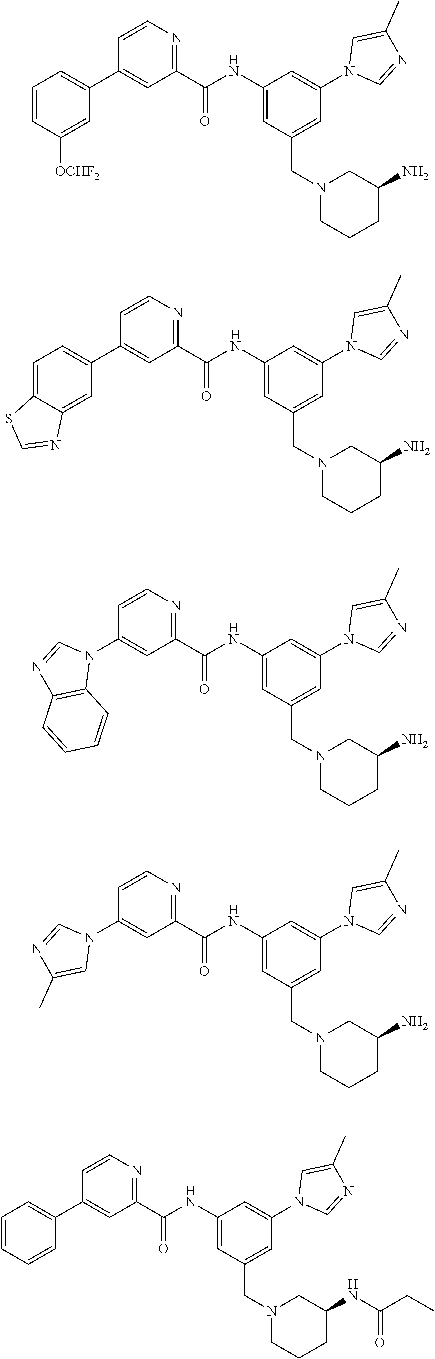 [SMILES: Cc1cn(-c2cc(CN3CCC[C@H](N)C3)cc(NC(=O)c3cc(-c4cccc(OC(F)F)c4)ccn3)c2)cn1 ; Cc1cn(-c2cc(CN3CCC[C@H](N)C3)cc(NC(=O)c3cc(-c4ccc5scnc5c4)ccn3)c2)cn1 ; Cc1cn(-c2cc(CN3CCC[C@H](N)C3)cc(NC(=O)c3cc(-n4cnc5ccccc54)ccn3)c2)cn1 ; Cc1cn(-c2cc(CN3CCC[C@H](N)C3)cc(NC(=O)c3cc(-n4cnc(C)c4)ccn3)c2)cn1 ; CCC(=O)N[C@H]1CCCN(Cc2cc(NC(=O)c3cc(-c4ccccc4)ccn3)cc(-n3cnc(C)c3)c2)C1] 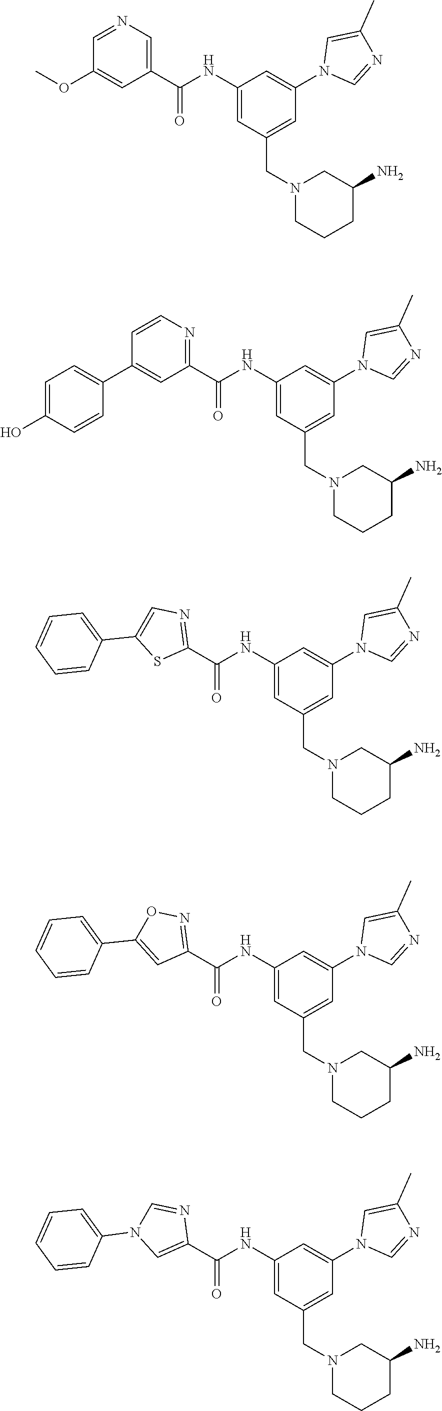 [SMILES: COc1cncc(C(=O)Nc2cc(CN3CCC[C@H](N)C3)cc(-n3cnc(C)c3)c2)c1 ; Cc1cn(-c2cc(CN3CCC[C@H](N)C3)cc(NC(=O)c3cc(-c4ccc(O)cc4)ccn3)c2)cn1 ; Cc1cn(-c2cc(CN3CCC[C@H](N)C3)cc(NC(=O)c3ncc(-c4ccccc4)s3)c2)cn1 ; Cc1cn(-c2cc(CN3CCC[C@H](N)C3)cc(NC(=O)c3cc(-c4ccccc4)on3)c2)cn1 ; Cc1cn(-c2cc(CN3CCC[C@H](N)C3)cc(NC(=O)c3cn(-c4ccccc4)cn3)c2)cn1] 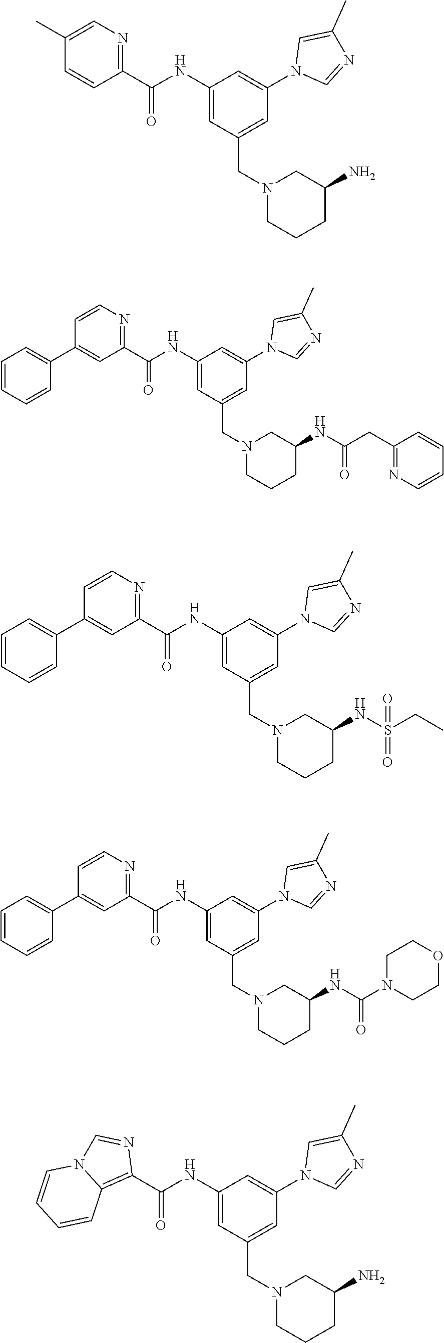 [SMILES: Cc1ccc(C(=O)Nc2cc(CN3CCC[C@H](N)C3)cc(-n3cnc(C)c3)c2)nc1 ; Cc1cn(-c2cc(CN3CCC[C@H](NC(=O)Cc4ccccn4)C3)cc(NC(=O)c3cc(-c4ccccc4)ccn3)c2)cn1 ; CCS(=O)(=O)N[C@H]1CCCN(Cc2cc(NC(=O)c3cc(-c4ccccc4)ccn3)cc(-n3cnc(C)c3)c2)C1 ; Cc1cn(-c2cc(CN3CCC[C@H](NC(=O)N4CCOCC4)C3)cc(NC(=O)c3cc(-c4ccccc4)ccn3)c2)cn1 ; Cc1cn(-c2cc(CN3CCC[C@H](N)C3)cc(NC(=O)c3ncn4ccccc34)c2)cn1] 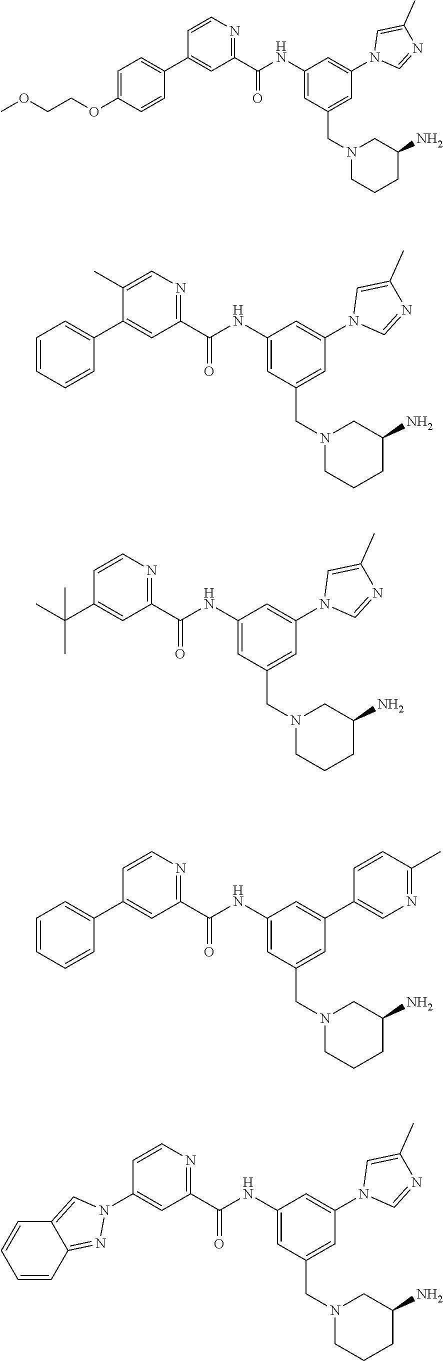 [SMILES: COCCOc1ccc(-c2ccnc(C(=O)Nc3cc(CN4CCC[C@H](N)C4)cc(-n4cnc(C)c4)c3)c2)cc1 ; Cc1cn(-c2cc(CN3CCC[C@H](N)C3)cc(NC(=O)c3cc(-c4ccccc4)c(C)cn3)c2)cn1 ; Cc1cn(-c2cc(CN3CCC[C@H](N)C3)cc(NC(=O)c3cc(C(C)(C)C)ccn3)c2)cn1 ; Cc1ccc(-c2cc(CN3CCC[C@H](N)C3)cc(NC(=O)c3cc(-c4ccccc4)ccn3)c2)cn1 ; Cc1cn(-c2cc(CN3CCC[C@H](N)C3)cc(NC(=O)c3cc(-n4cc5ccccc5n4)ccn3)c2)cn1] 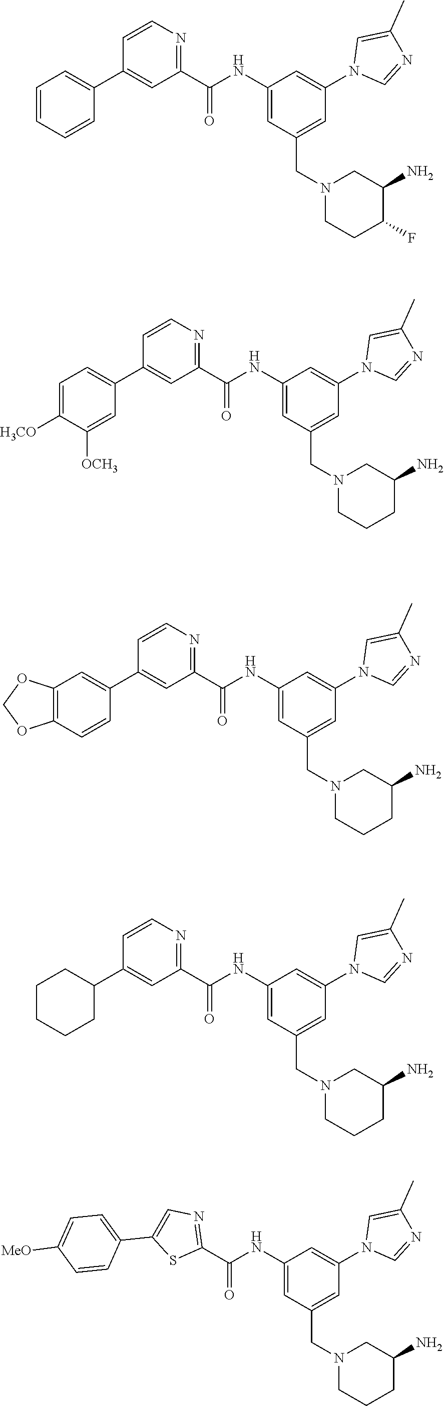 [SMILES: Cc1cn(-c2cc(CN3CC[C@@H](F)[C@H](N)C3)cc(NC(=O)c3cc(-c4ccccc4)ccn3)c2)cn1 ; COc1ccc(-c2ccnc(C(=O)Nc3cc(CN4CCC[C@H](N)C4)cc(-n4cnc(C)c4)c3)c2)cc1OC ; Cc1cn(-c2cc(CN3CCC[C@H](N)C3)cc(NC(=O)c3cc(-c4ccc5c(c4)OCO5)ccn3)c2)cn1 ; Cc1cn(-c2cc(CN3CCC[C@H](N)C3)cc(NC(=O)c3cc(C4CCCCC4)ccn3)c2)cn1 ; COc1ccc(-c2cnc(C(=O)Nc3cc(CN4CCC[C@H](N)C4)cc(-n4cnc(C)c4)c3)s2)cc1] 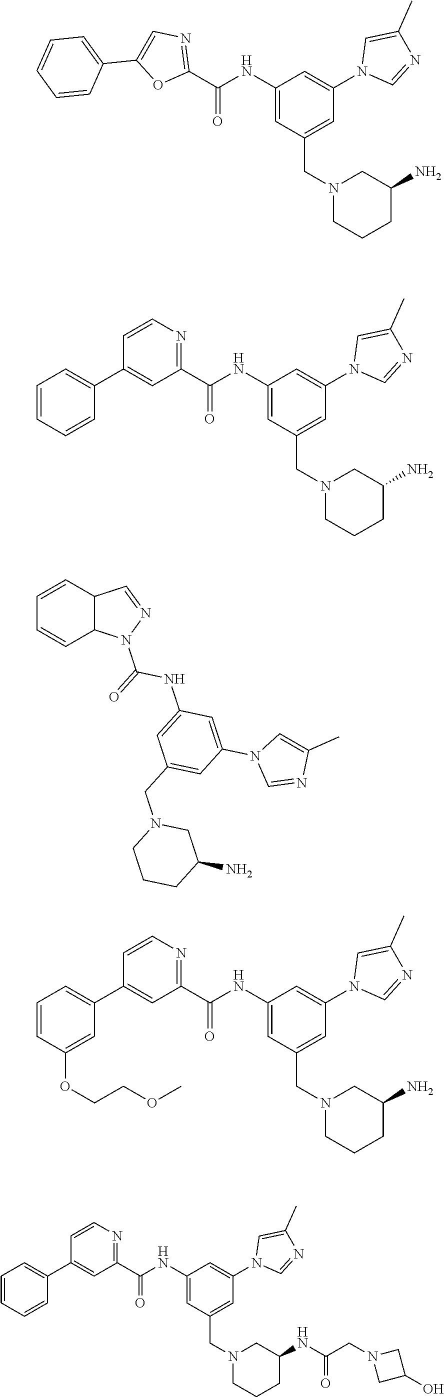 [SMILES: Cc1cn(-c2cc(CN3CCC[C@H](N)C3)cc(NC(=O)c3ncc(-c4ccccc4)o3)c2)cn1 ; Cc1cn(-c2cc(CN3CCC[C@@H](N)C3)cc(NC(=O)c3cc(-c4ccccc4)ccn3)c2)cn1 ; Cc1cn(-c2cc(CN3CCC[C@H](N)C3)cc(NC(=O)N3N=CC4C=CC=CC43)c2)cn1 ; COCCOc1cccc(-c2ccnc(C(=O)Nc3cc(CN4CCC[C@H](N)C4)cc(-n4cnc(C)c4)c3)c2)c1 ; Cc1cn(-c2cc(CN3CCC[C@H](NC(=O)CN4CC(O)C4)C3)cc(NC(=O)c3cc(-c4ccccc4)ccn3)c2)cn1] 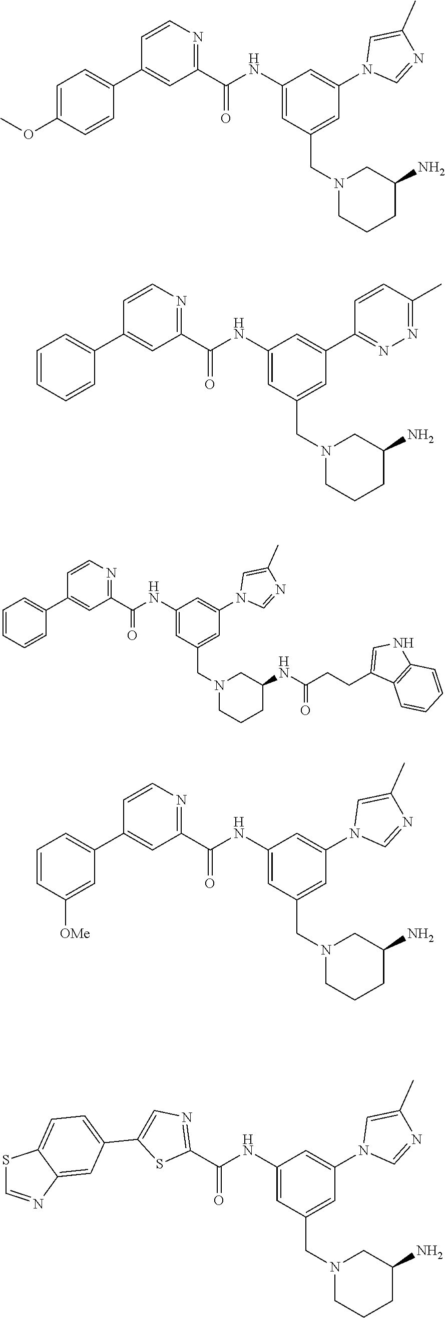 [SMILES: COc1ccc(-c2ccnc(C(=O)Nc3cc(CN4CCC[C@H](N)C4)cc(-n4cnc(C)c4)c3)c2)cc1 ; Cc1ccc(-c2cc(CN3CCC[C@H](N)C3)cc(NC(=O)c3cc(-c4ccccc4)ccn3)c2)nn1 ; Cc1cn(-c2cc(CN3CCC[C@H](NC(=O)CCc4c[nH]c5ccccc45)C3)cc(NC(=O)c3cc(-c4ccccc4)ccn3)c2)cn1 ; COc1cccc(-c2ccnc(C(=O)Nc3cc(CN4CCC[C@H](N)C4)cc(-n4cnc(C)c4)c3)c2)c1 ; Cc1cn(-c2cc(CN3CCC[C@H](N)C3)cc(NC(=O)c3ncc(-c4ccc5scnc5c4)s3)c2)cn1] 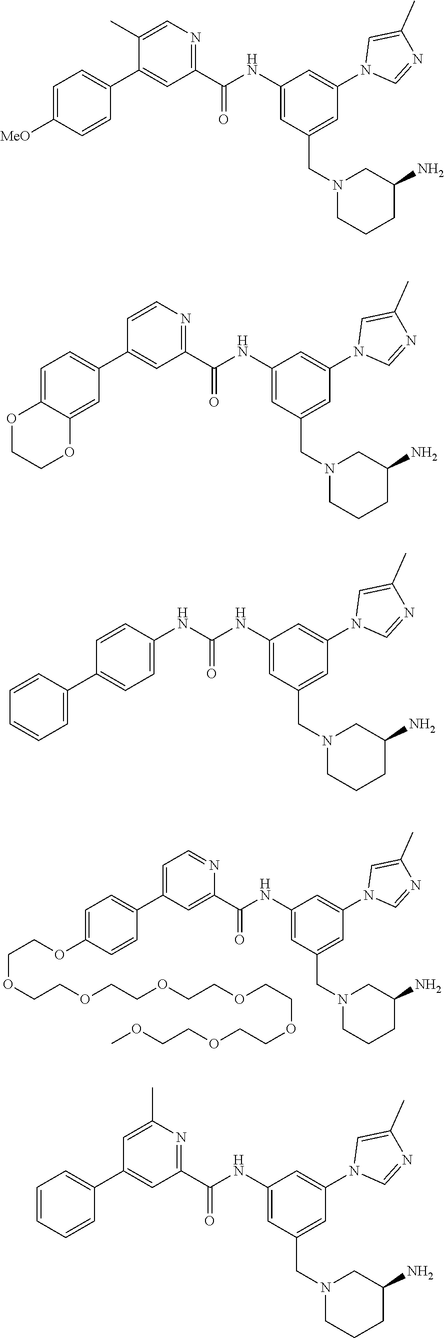 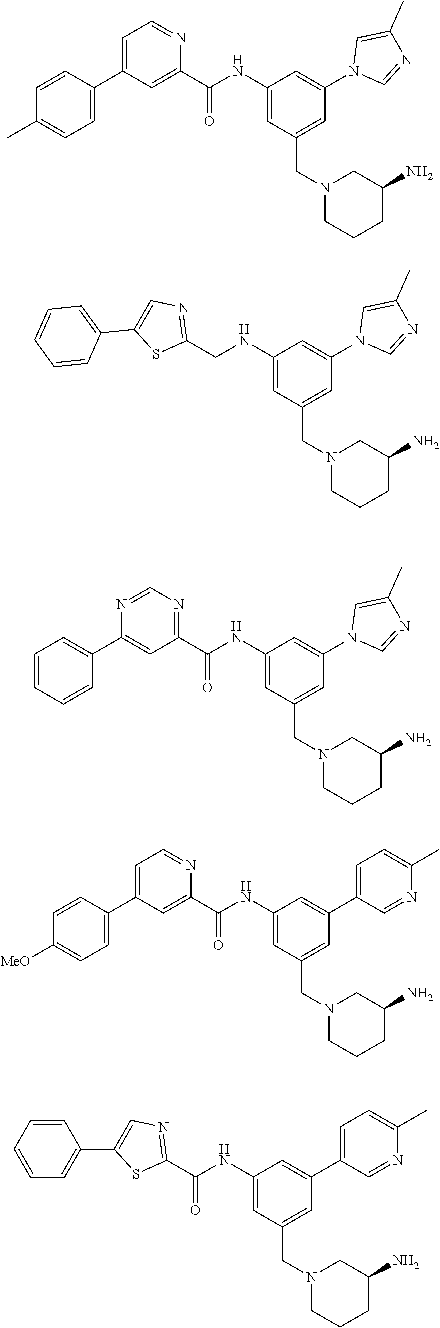 [SMILES: Cc1ccc(-c2ccnc(C(=O)Nc3cc(CN4CCC[C@H](N)C4)cc(-n4cnc(C)c4)c3)c2)cc1 ; Cc1cn(-c2cc(CN3CCC[C@H](N)C3)cc(NCc3ncc(-c4ccccc4)s3)c2)cn1 ; Cc1cn(-c2cc(CN3CCC[C@H](N)C3)cc(NC(=O)c3cc(-c4ccccc4)ncn3)c2)cn1 ; COc1ccc(-c2ccnc(C(=O)Nc3cc(CN4CCC[C@H](N)C4)cc(-c4ccc(C)nc4)c3)c2)cc1 ; Cc1ccc(-c2cc(CN3CCC[C@H](N)C3)cc(NC(=O)c3ncc(-c4ccccc4)s3)c2)cn1] 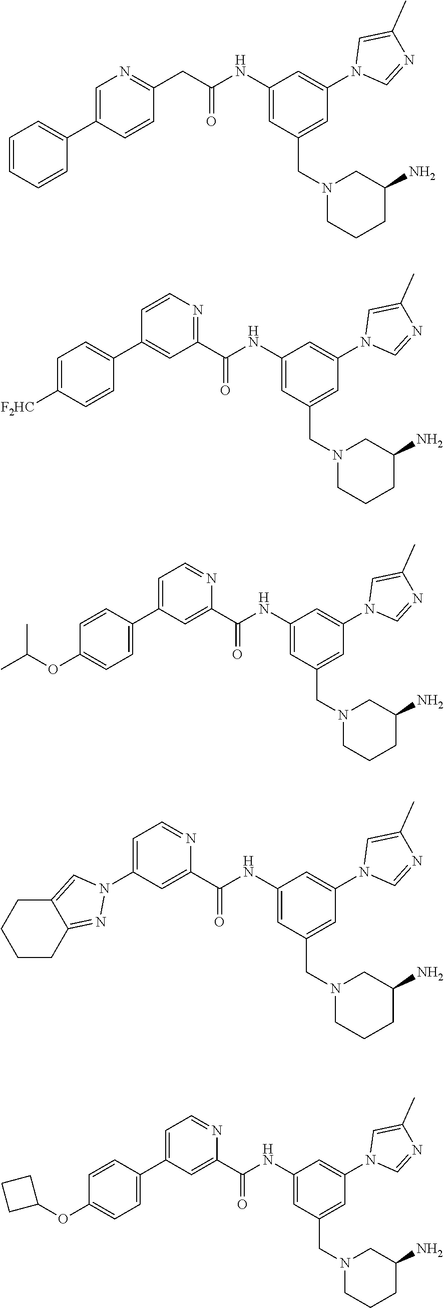 [SMILES: Cc1cn(-c2cc(CN3CCC[C@H](N)C3)cc(NC(=O)Cc3ccc(-c4ccccc4)cn3)c2)cn1 ; Cc1cn(-c2cc(CN3CCC[C@H](N)C3)cc(NC(=O)c3cc(-c4ccc(C(F)F)cc4)ccn3)c2)cn1 ; Cc1cn(-c2cc(CN3CCC[C@H](N)C3)cc(NC(=O)c3cc(-c4ccc(OC(C)C)cc4)ccn3)c2)cn1 ; Cc1cn(-c2cc(CN3CCC[C@H](N)C3)cc(NC(=O)c3cc(-n4cc5c(n4)CCCC5)ccn3)c2)cn1 ; Cc1cn(-c2cc(CN3CCC[C@H](N)C3)cc(NC(=O)c3cc(-c4ccc(OC5CCC5)cc4)ccn3)c2)cn1] 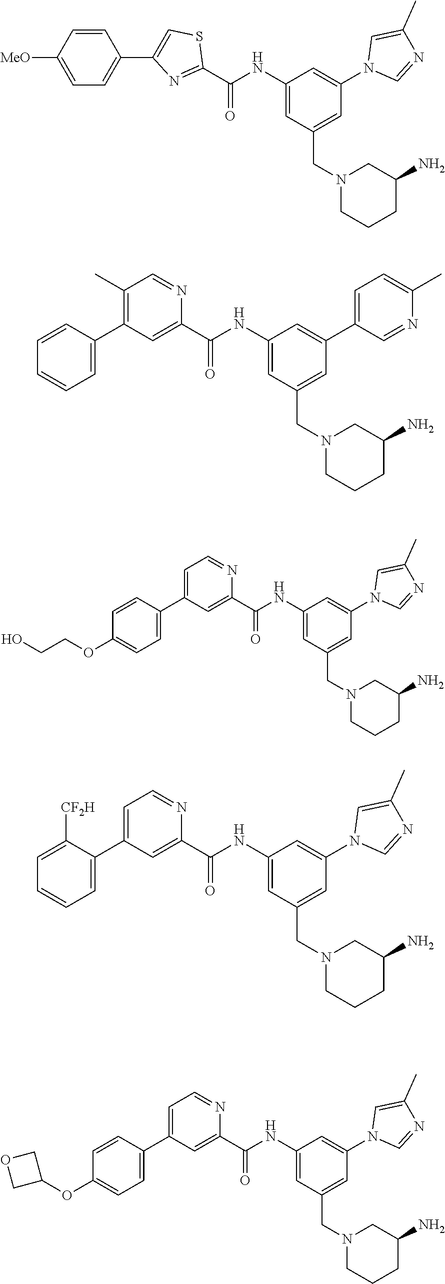 [SMILES: COc1ccc(-c2csc(C(=O)Nc3cc(CN4CCC[C@H](N)C4)cc(-n4cnc(C)c4)c3)n2)cc1 ; Cc1ccc(-c2cc(CN3CCC[C@H](N)C3)cc(NC(=O)c3cc(-c4ccccc4)c(C)cn3)c2)cn1 ; Cc1cn(-c2cc(CN3CCC[C@H](N)C3)cc(NC(=O)c3cc(-c4ccc(OCCO)cc4)ccn3)c2)cn1 ; Cc1cn(-c2cc(CN3CCC[C@H](N)C3)cc(NC(=O)c3cc(-c4ccccc4C(F)F)ccn3)c2)cn1 ; Cc1cn(-c2cc(CN3CCC[C@H](N)C3)cc(NC(=O)c3cc(-c4ccc(OC5COC5)cc4)ccn3)c2)cn1] 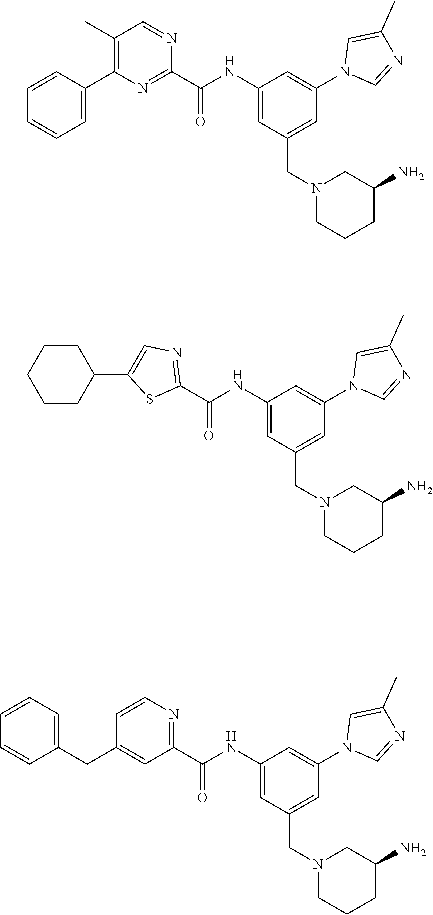 [SMILES: Cc1cn(-c2cc(CN3CCC[C@H](N)C3)cc(NC(=O)c3ncc(C)c(-c4ccccc4)n3)c2)cn1 ; Cc1cn(-c2cc(CN3CCC[C@H](N)C3)cc(NC(=O)c3ncc(C4CCCCC4)s3)c2)cn1 ; Cc1cn(-c2cc(CN3CCC[C@H](N)C3)cc(NC(=O)c3cc(Cc4ccccc4)ccn3)c2)cn1]
or a salt, solvate, prodrug or polymorph thereof.

10. A compound selected from the group consisting of: 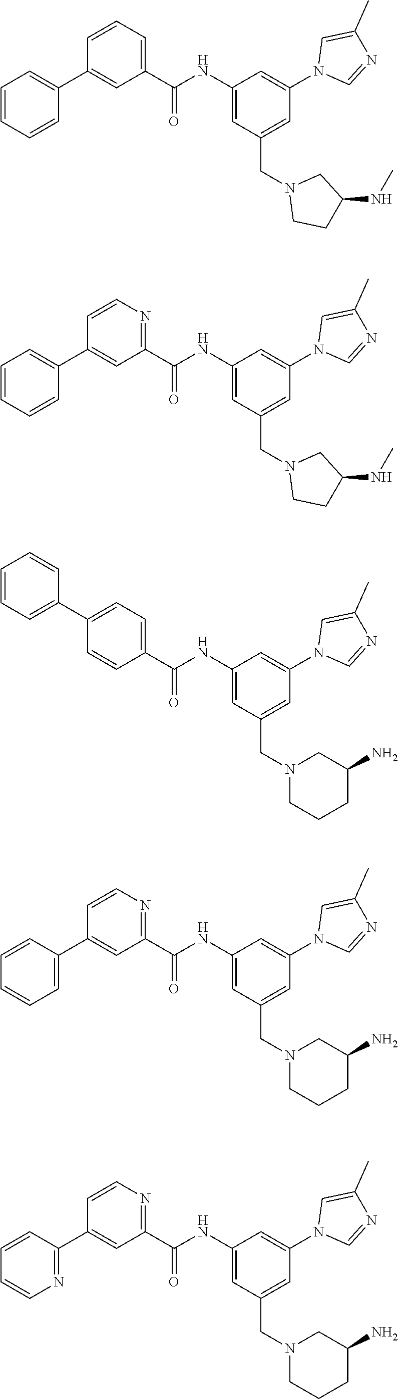 [SMILES: CN[C@H]1CCN(Cc2cc(NC(=O)c3cccc(-c4ccccc4)c3)cc(-n3cnc(C)c3)c2)C1 ; CN[C@H]1CCN(Cc2cc(NC(=O)c3cc(-c4ccccc4)ccn3)cc(-n3cnc(C)c3)c2)C1 ; Cc1cn(-c2cc(CN3CCC[C@H](N)C3)cc(NC(=O)c3ccc(-c4ccccc4)cc3)c2)cn1 ; Cc1cn(-c2cc(CN3CCC[C@H](N)C3)cc(NC(=O)c3cc(-c4ccccc4)ccn3)c2)cn1 ; Cc1cn(-c2cc(CN3CCC[C@H](N)C3)cc(NC(=O)c3cc(-c4ccccn4)ccn3)c2)cn1] 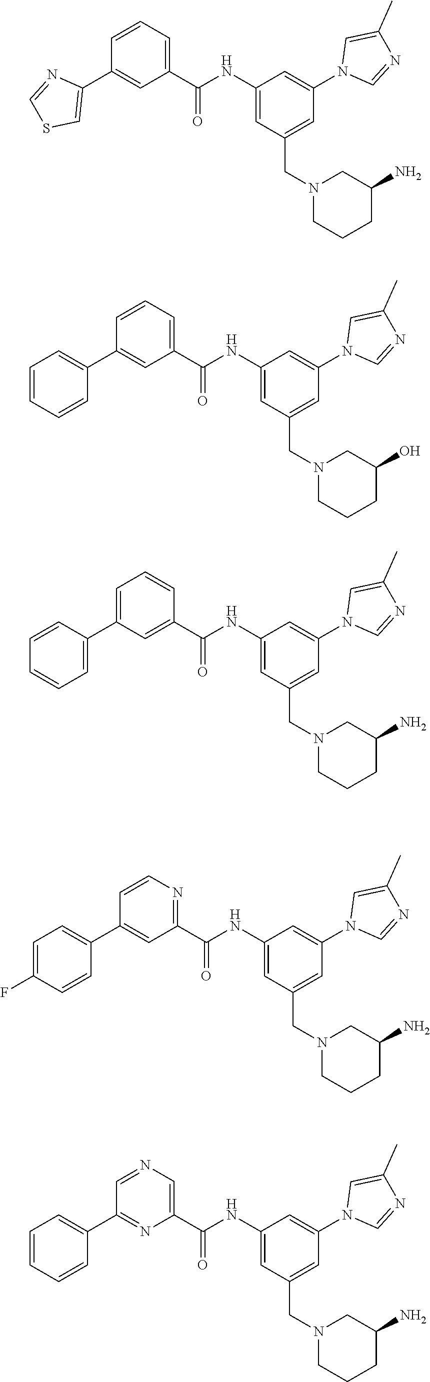 [SMILES: Cc1cn(-c2cc(CN3CCC[C@H](N)C3)cc(NC(=O)c3cccc(-c4cscn4)c3)c2)cn1 ; Cc1cn(-c2cc(CN3CCC[C@H](O)C3)cc(NC(=O)c3cccc(-c4ccccc4)c3)c2)cn1 ; Cc1cn(-c2cc(CN3CCC[C@H](N)C3)cc(NC(=O)c3cccc(-c4ccccc4)c3)c2)cn1 ; Cc1cn(-c2cc(CN3CCC[C@H](N)C3)cc(NC(=O)c3cc(-c4ccc(F)cc4)ccn3)c2)cn1 ; Cc1cn(-c2cc(CN3CCC[C@H](N)C3)cc(NC(=O)c3cncc(-c4ccccc4)n3)c2)cn1] 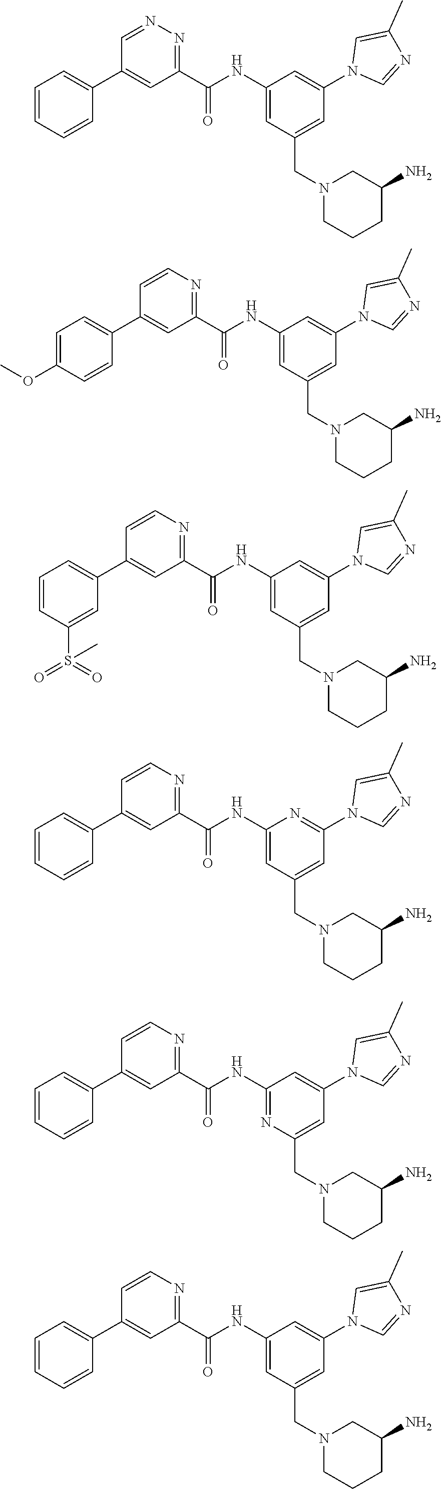 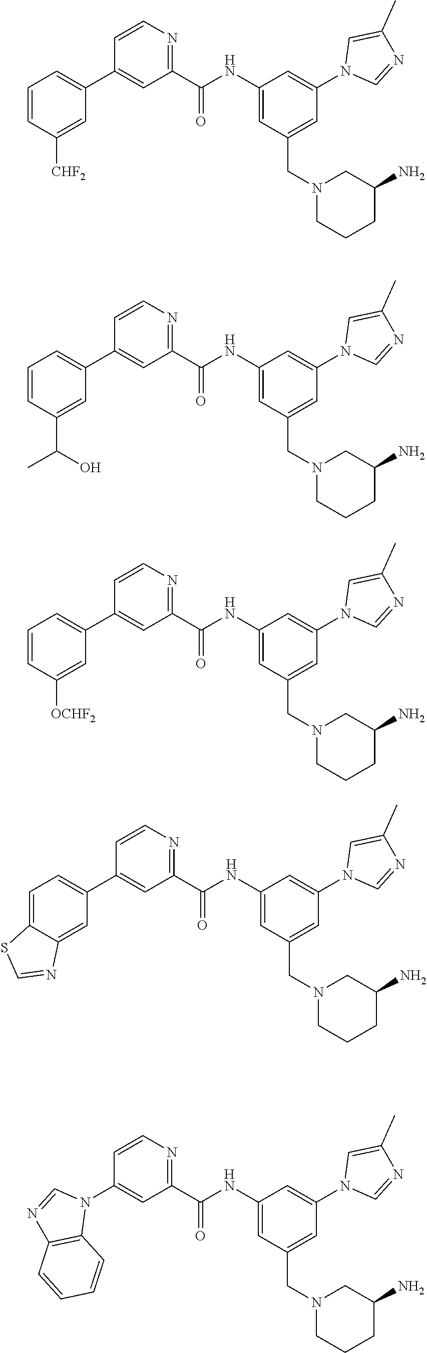 [SMILES: Cc1cn(-c2cc(CN3CCC[C@H](N)C3)cc(NC(=O)c3cc(-c4cccc(C(F)F)c4)ccn3)c2)cn1 ; Cc1cn(-c2cc(CN3CCC[C@H](N)C3)cc(NC(=O)c3cc(-c4cccc(C(C)O)c4)ccn3)c2)cn1 ; Cc1cn(-c2cc(CN3CCC[C@H](N)C3)cc(NC(=O)c3cc(-c4cccc(OC(F)F)c4)ccn3)c2)cn1 ; Cc1cn(-c2cc(CN3CCC[C@H](N)C3)cc(NC(=O)c3cc(-c4ccc5scnc5c4)ccn3)c2)cn1 ; Cc1cn(-c2cc(CN3CCC[C@H](N)C3)cc(NC(=O)c3cc(-n4cnc5ccccc54)ccn3)c2)cn1] 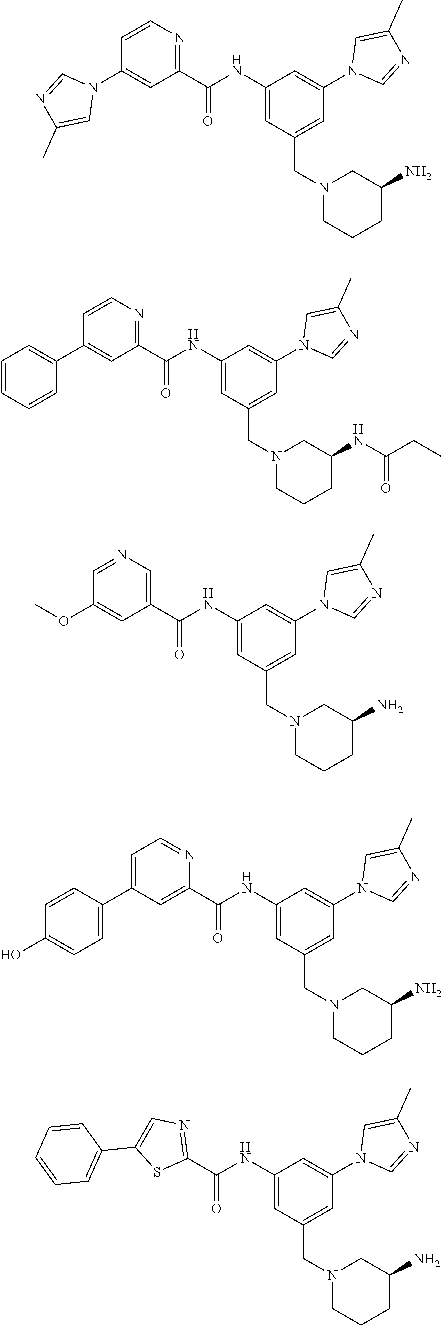 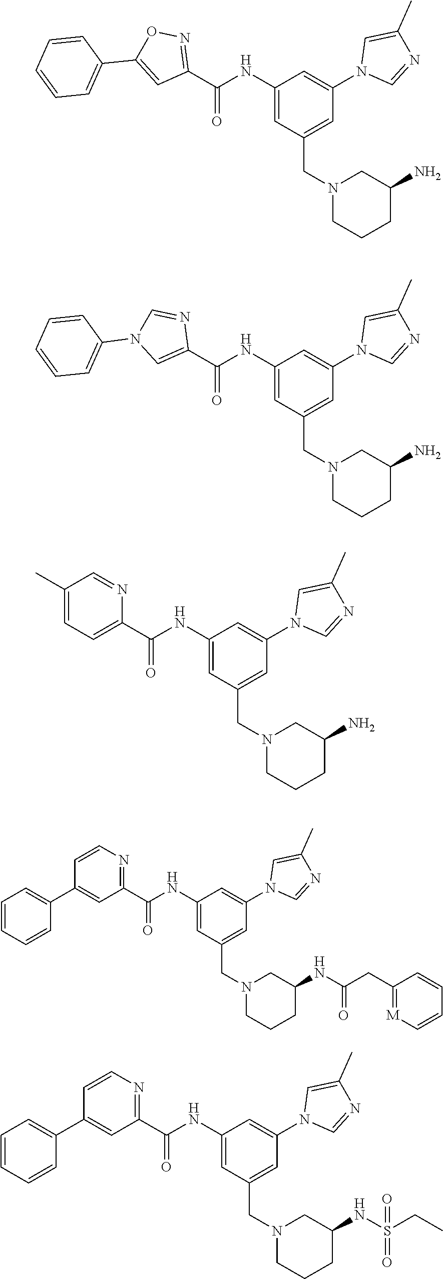 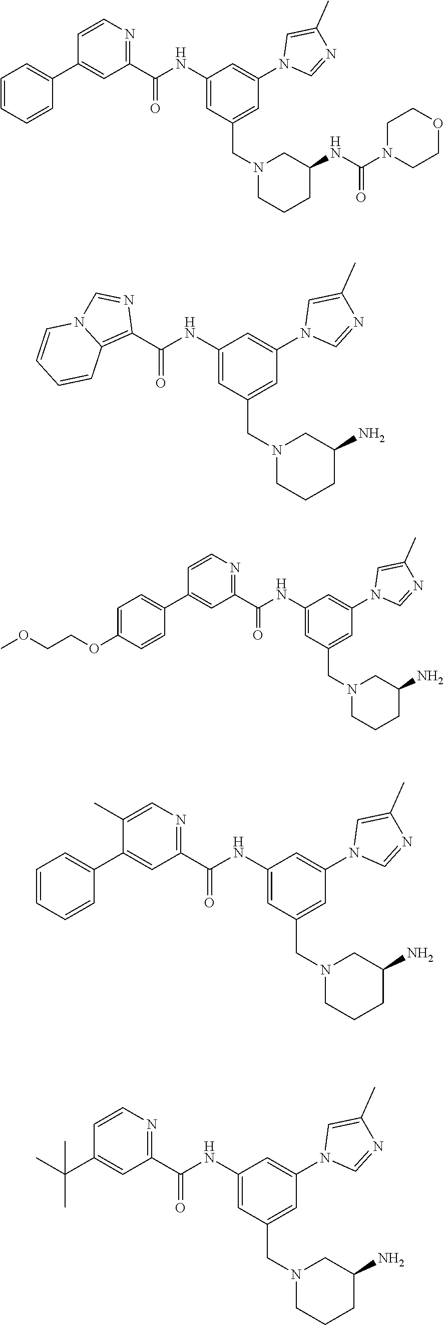 [SMILES: Cc1cn(-c2cc(CN3CCC[C@H](NC(=O)N4CCOCC4)C3)cc(NC(=O)c3cc(-c4ccccc4)ccn3)c2)cn1 ; Cc1cn(-c2cc(CN3CCC[C@H](N)C3)cc(NC(=O)c3ncn4ccccc34)c2)cn1 ; COCCOc1ccc(-c2ccnc(C(=O)Nc3cc(CN4CCC[C@H](N)C4)cc(-n4cnc(C)c4)c3)c2)cc1 ; Cc1cn(-c2cc(CN3CCC[C@H](N)C3)cc(NC(=O)c3cc(-c4ccccc4)c(C)cn3)c2)cn1 ; Cc1cn(-c2cc(CN3CCC[C@H](N)C3)cc(NC(=O)c3cc(C(C)(C)C)ccn3)c2)cn1] 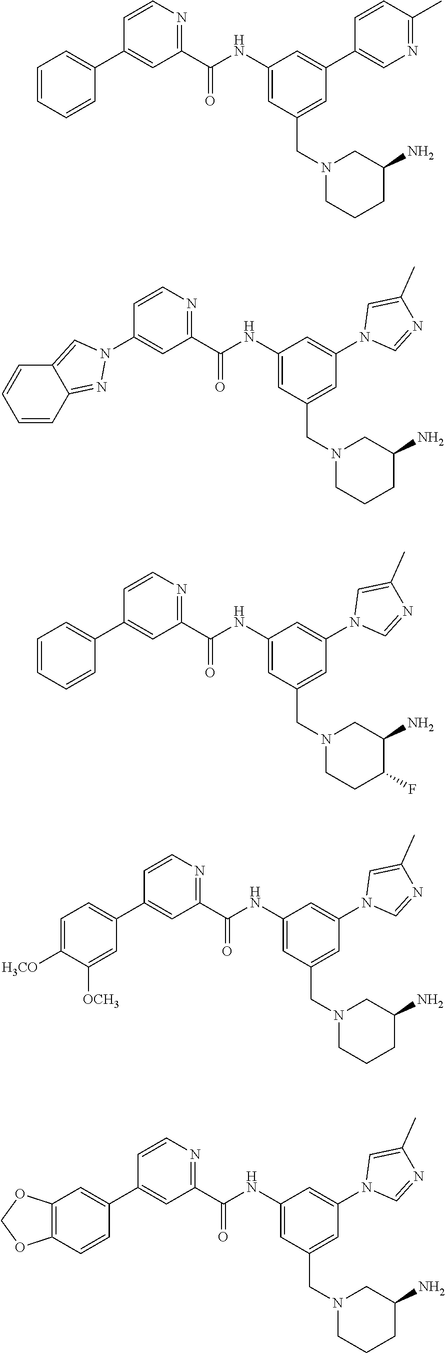 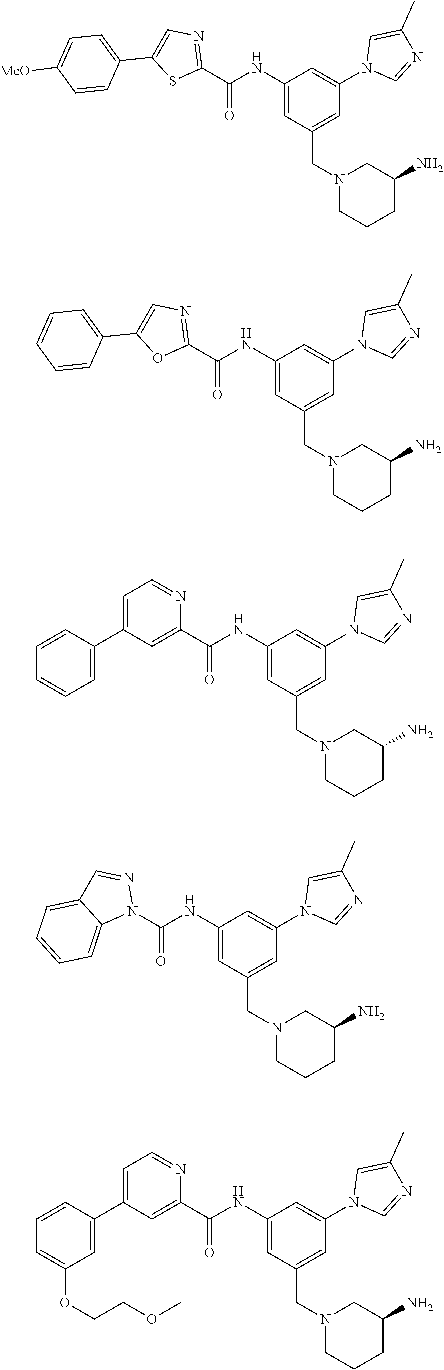 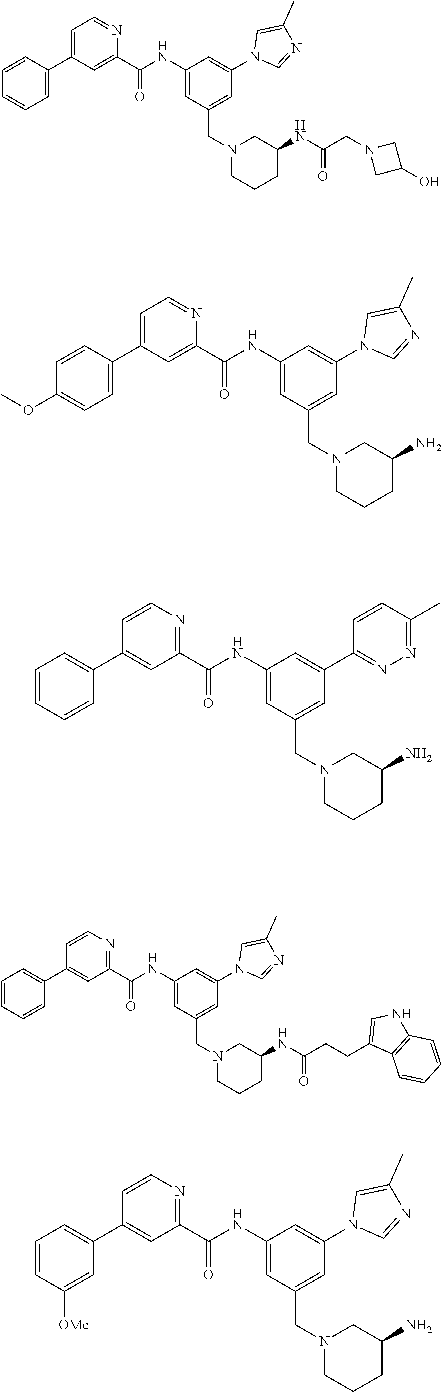 [SMILES: Cc1cn(-c2cc(CN3CCC[C@H](NC(=O)CN4CC(O)C4)C3)cc(NC(=O)c3cc(-c4ccccc4)ccn3)c2)cn1 ; COc1ccc(-c2ccnc(C(=O)Nc3cc(CN4CCC[C@H](N)C4)cc(-n4cnc(C)c4)c3)c2)cc1 ; Cc1ccc(-c2cc(CN3CCC[C@H](N)C3)cc(NC(=O)c3cc(-c4ccccc4)ccn3)c2)nn1 ; Cc1cn(-c2cc(CN3CCC[C@H](NC(=O)CCc4c[nH]c5ccccc45)C3)cc(NC(=O)c3cc(-c4ccccc4)ccn3)c2)cn1 ; COc1cccc(-c2ccnc(C(=O)Nc3cc(CN4CCC[C@H](N)C4)cc(-n4cnc(C)c4)c3)c2)c1] 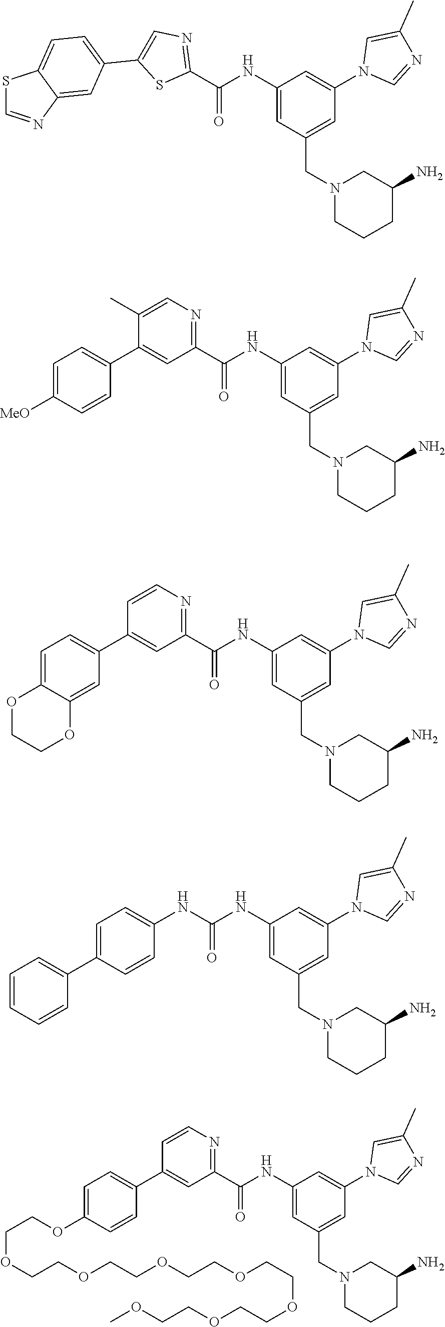 [SMILES: Cc1cn(-c2cc(CN3CCC[C@H](N)C3)cc(NC(=O)c3ncc(-c4ccc5scnc5c4)s3)c2)cn1 ; COc1ccc(-c2cc(C(=O)Nc3cc(CN4CCC[C@H](N)C4)cc(-n4cnc(C)c4)c3)ncc2C)cc1 ; Cc1cn(-c2cc(CN3CCC[C@H](N)C3)cc(NC(=O)c3cc(-c4ccc5c(c4)OCCO5)ccn3)c2)cn1 ; Cc1cn(-c2cc(CN3CCC[C@H](N)C3)cc(NC(=O)Nc3ccc(-c4ccccc4)cc3)c2)cn1 ; COCCOCCOCCOCCOCCOCCOCCOc1ccc(-c2ccnc(C(=O)Nc3cc(CN4CCC[C@H](N)C4)cc(-n4cnc(C)c4)c3)c2)cc1] 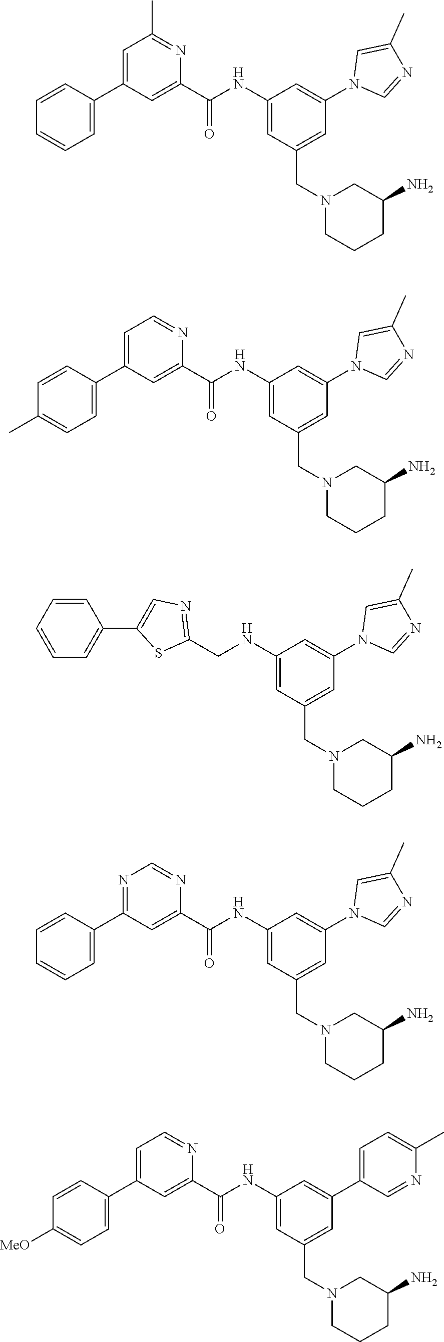 [SMILES: Cc1cn(-c2cc(CN3CCC[C@H](N)C3)cc(NC(=O)c3cc(-c4ccccc4)cc(C)n3)c2)cn1 ; Cc1ccc(-c2ccnc(C(=O)Nc3cc(CN4CCC[C@H](N)C4)cc(-n4cnc(C)c4)c3)c2)cc1 ; Cc1cn(-c2cc(CN3CCC[C@H](N)C3)cc(NCc3ncc(-c4ccccc4)s3)c2)cn1 ; Cc1cn(-c2cc(CN3CCC[C@H](N)C3)cc(NC(=O)c3cc(-c4ccccc4)ncn3)c2)cn1 ; COc1ccc(-c2ccnc(C(=O)Nc3cc(CN4CCC[C@H](N)C4)cc(-c4ccc(C)nc4)c3)c2)cc1] 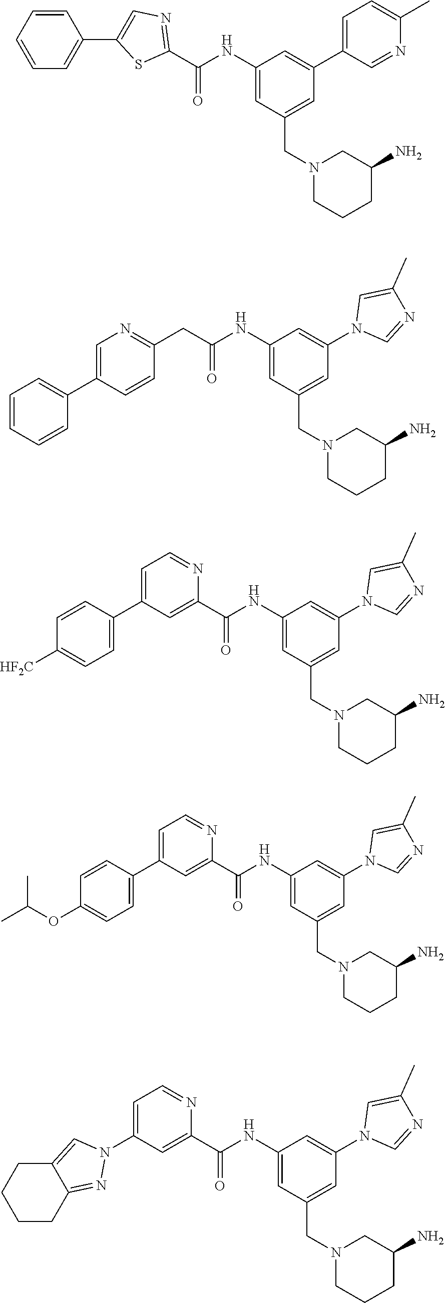 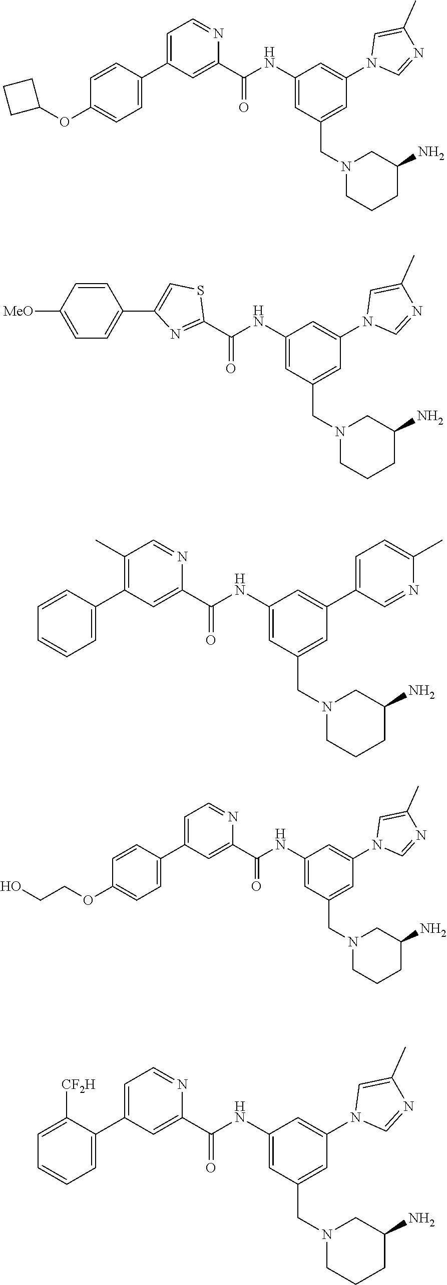 [SMILES: Cc1cn(-c2cc(CN3CCC[C@H](N)C3)cc(NC(=O)c3cc(-c4ccc(OC5CCC5)cc4)ccn3)c2)cn1 ; COc1ccc(-c2csc(C(=O)Nc3cc(CN4CCC[C@H](N)C4)cc(-n4cnc(C)c4)c3)n2)cc1 ; Cc1ccc(-c2cc(CN3CCC[C@H](N)C3)cc(NC(=O)c3cc(-c4ccccc4)c(C)cn3)c2)cn1 ; Cc1cn(-c2cc(CN3CCC[C@H](N)C3)cc(NC(=O)c3cc(-c4ccc(OCCO)cc4)ccn3)c2)cn1 ; Cc1cn(-c2cc(CN3CCC[C@H](N)C3)cc(NC(=O)c3cc(-c4ccccc4C(F)F)ccn3)c2)cn1] 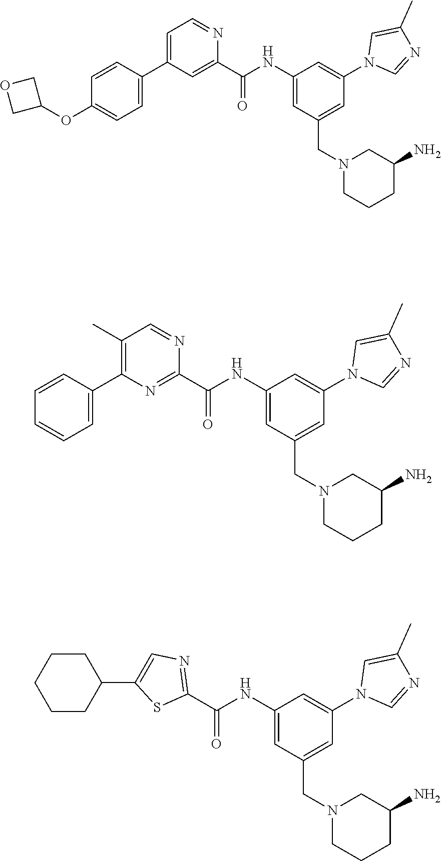 [SMILES: Cc1cn(-c2cc(CN3CCC[C@H](N)C3)cc(NC(=O)c3cc(-c4ccc(OC5COC5)cc4)ccn3)c2)cn1 ; Cc1cn(-c2cc(CN3CCC[C@H](N)C3)cc(NC(=O)c3ncc(C)c(-c4ccccc4)n3)c2)cn1 ; Cc1cn(-c2cc(CN3CCC[C@H](N)C3)cc(NC(=O)c3ncc(C4CCCCC4)s3)c2)cn1]
or a salt, solvate, prodrug or polymorph thereof.

11. A compound selected from the group consisting of: 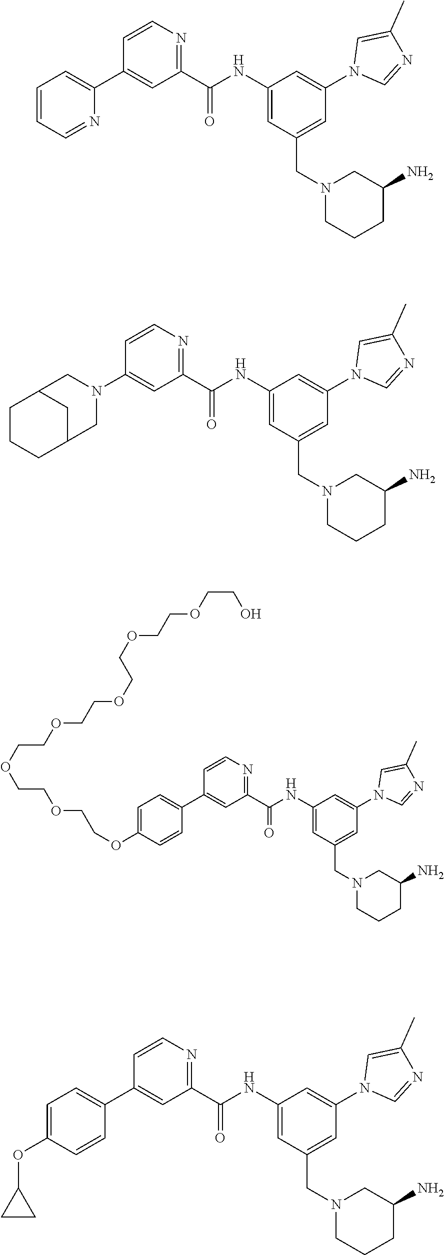 [SMILES: Cc1cn(-c2cc(CN3CCC[C@H](N)C3)cc(NC(=O)c3cc(-c4ccccn4)ccn3)c2)cn1 ; Cc1cn(-c2cc(CN3CCC[C@H](N)C3)cc(NC(=O)c3cc(N4CC5CCCC(C5)C4)ccn3)c2)cn1 ; Cc1cn(-c2cc(CN3CCC[C@H](N)C3)cc(NC(=O)c3cc(-c4ccc(OCCOCCOCCOCCOCCOCCOCCO)cc4)ccn3)c2)cn1 ; Cc1cn(-c2cc(CN3CCC[C@H](N)C3)cc(NC(=O)c3cc(-c4ccc(OC5CC5)cc4)ccn3)c2)cn1] 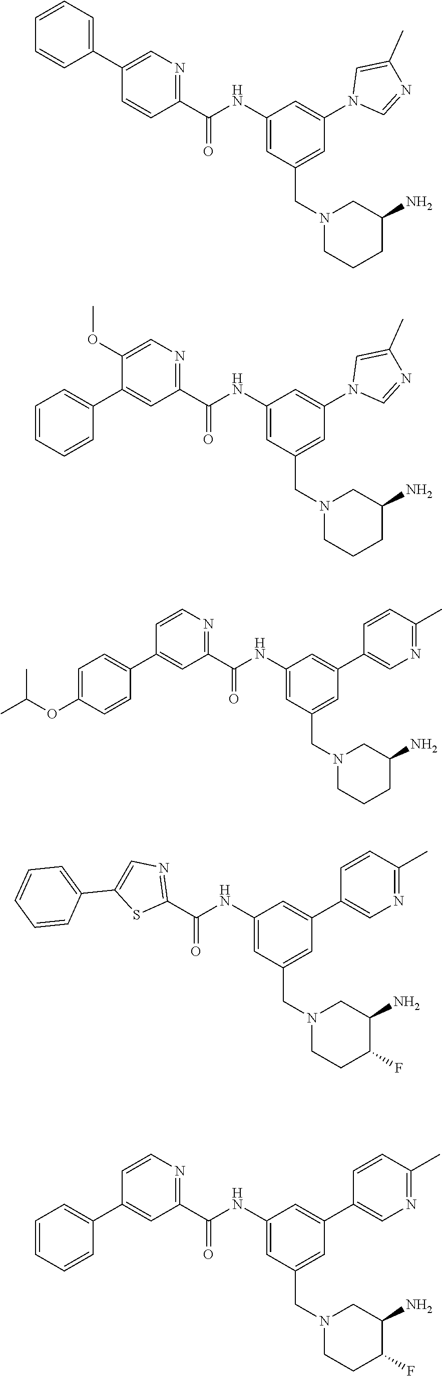 [SMILES: Cc1cn(-c2cc(CN3CCC[C@H](N)C3)cc(NC(=O)c3ccc(-c4ccccc4)cn3)c2)cn1 ; COc1cnc(C(=O)Nc2cc(CN3CCC[C@H](N)C3)cc(-n3cnc(C)c3)c2)cc1-c1ccccc1 ; Cc1ccc(-c2cc(CN3CCC[C@H](N)C3)cc(NC(=O)c3cc(-c4ccc(OC(C)C)cc4)ccn3)c2)cn1 ; Cc1ccc(-c2cc(CN3CC[C@@H](F)[C@H](N)C3)cc(NC(=O)c3ncc(-c4ccccc4)s3)c2)cn1 ; Cc1ccc(-c2cc(CN3CC[C@@H](F)[C@H](N)C3)cc(NC(=O)c3cc(-c4ccccc4)ccn3)c2)cn1] 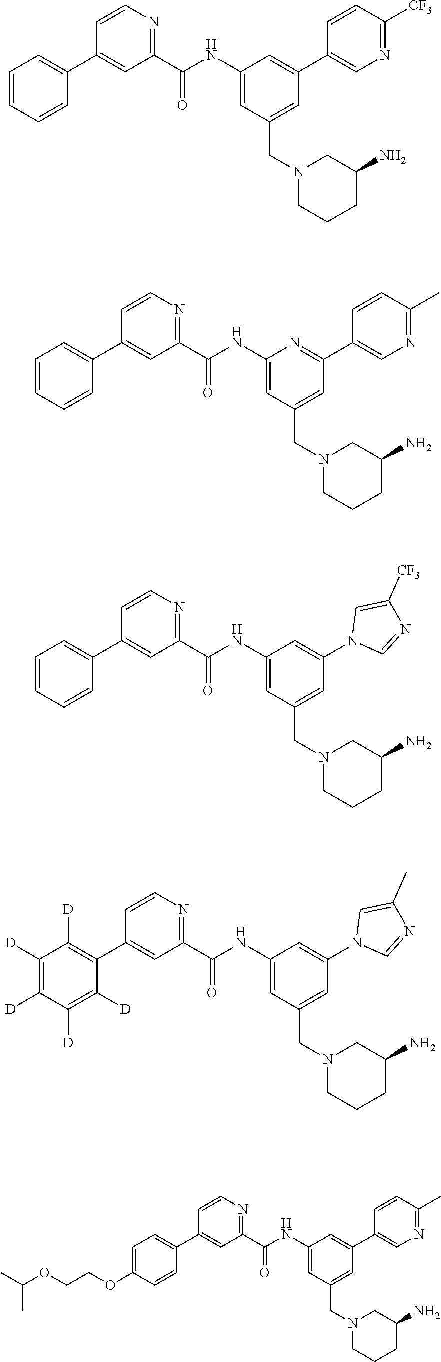 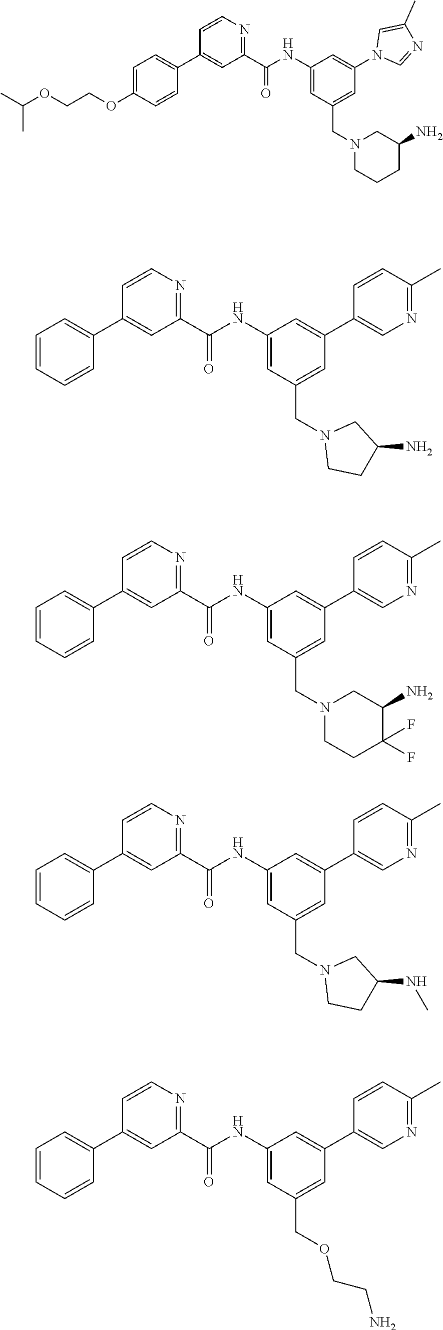 [SMILES: Cc1cn(-c2cc(CN3CCC[C@H](N)C3)cc(NC(=O)c3cc(-c4ccc(OCCOC(C)C)cc4)ccn3)c2)cn1 ; Cc1ccc(-c2cc(CN3CC[C@H](N)C3)cc(NC(=O)c3cc(-c4ccccc4)ccn3)c2)cn1 ; Cc1ccc(-c2cc(CN3CCC(F)(F)[C@H](N)C3)cc(NC(=O)c3cc(-c4ccccc4)ccn3)c2)cn1 ; CN[C@H]1CCN(Cc2cc(NC(=O)c3cc(-c4ccccc4)ccn3)cc(-c3ccc(C)nc3)c2)C1 ; Cc1ccc(-c2cc(COCCN)cc(NC(=O)c3cc(-c4ccccc4)ccn3)c2)cn1] 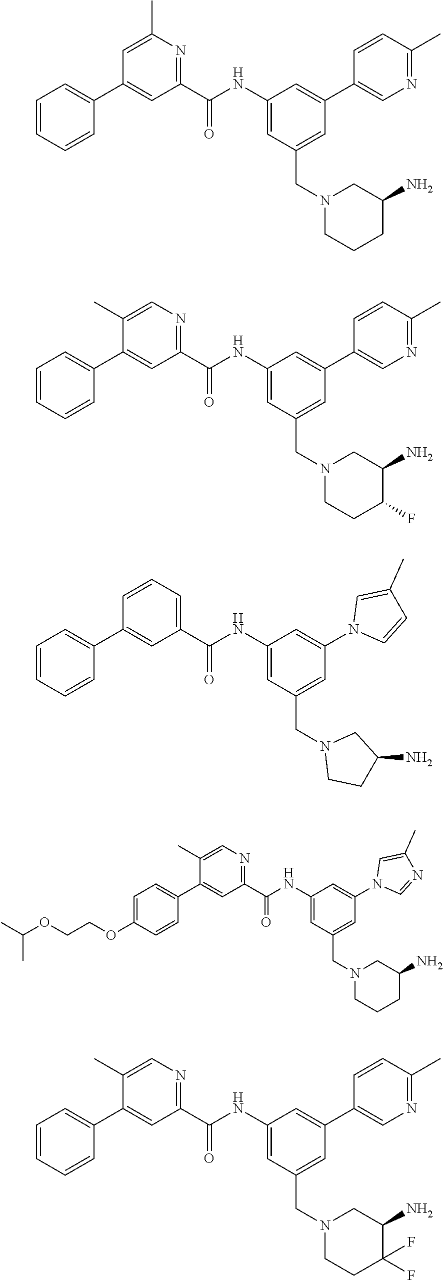 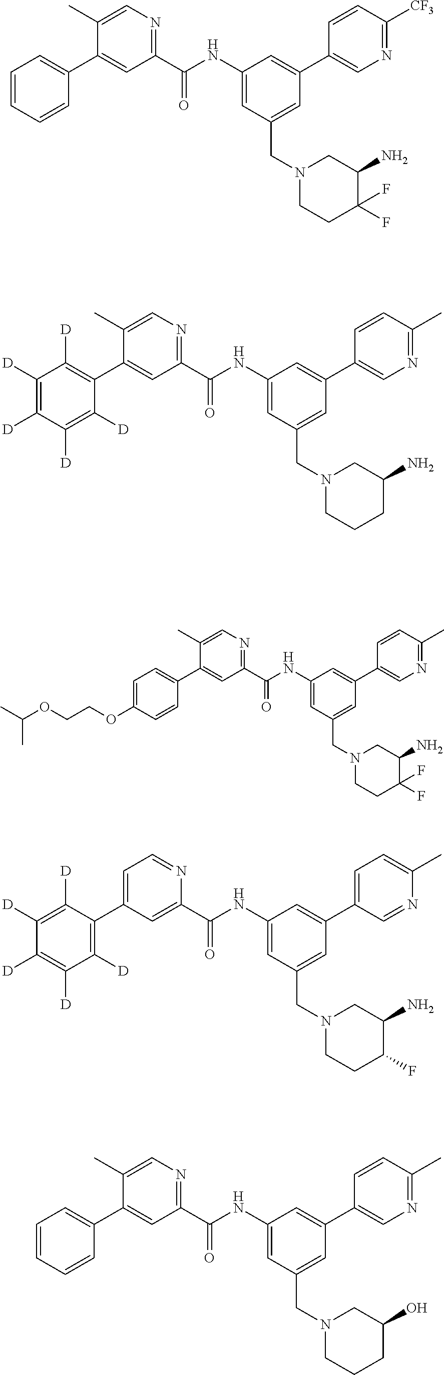 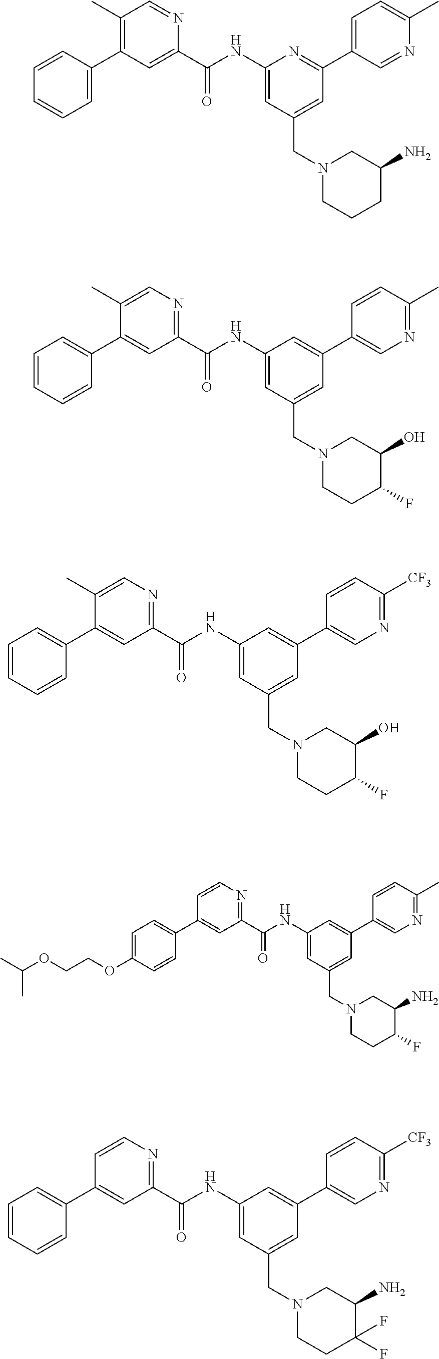 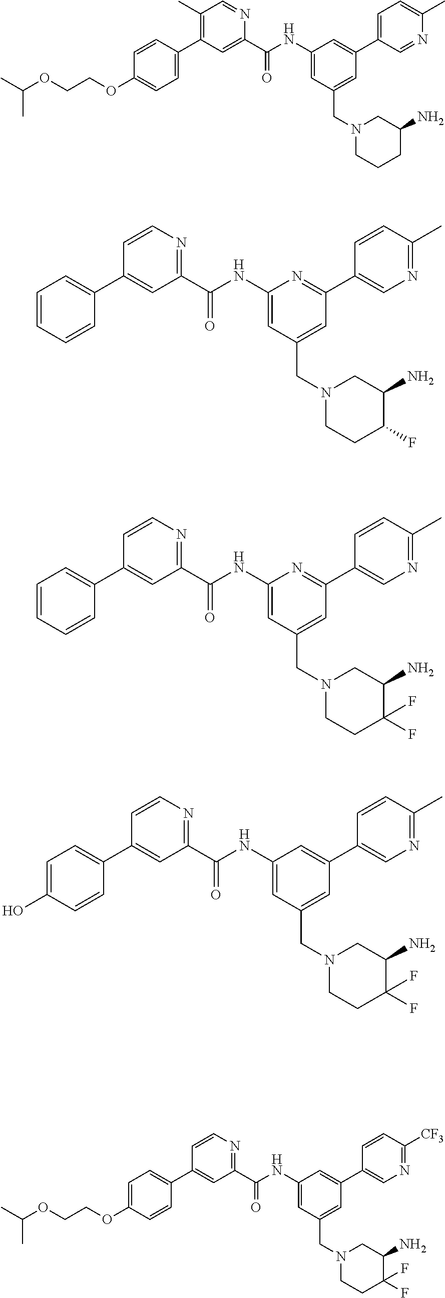 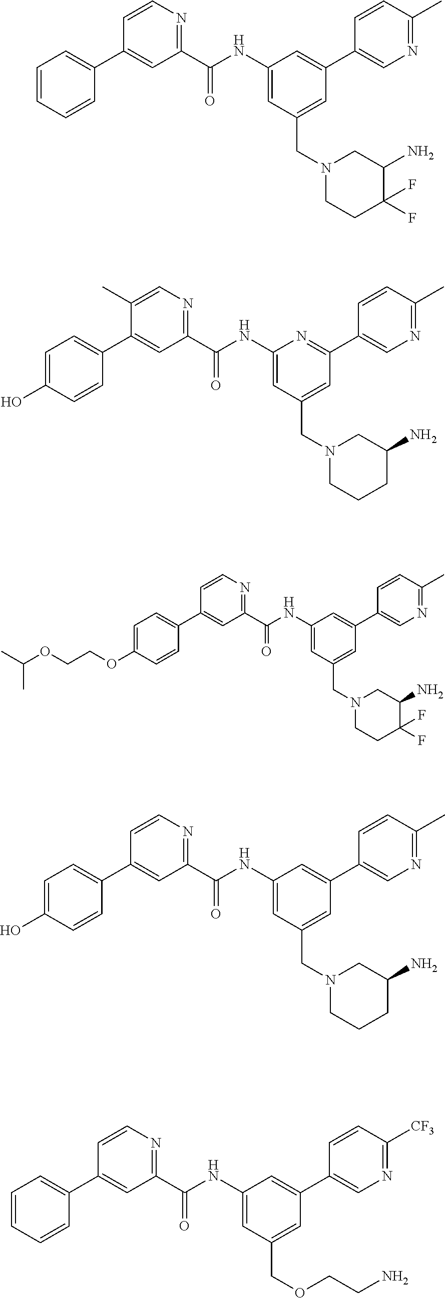 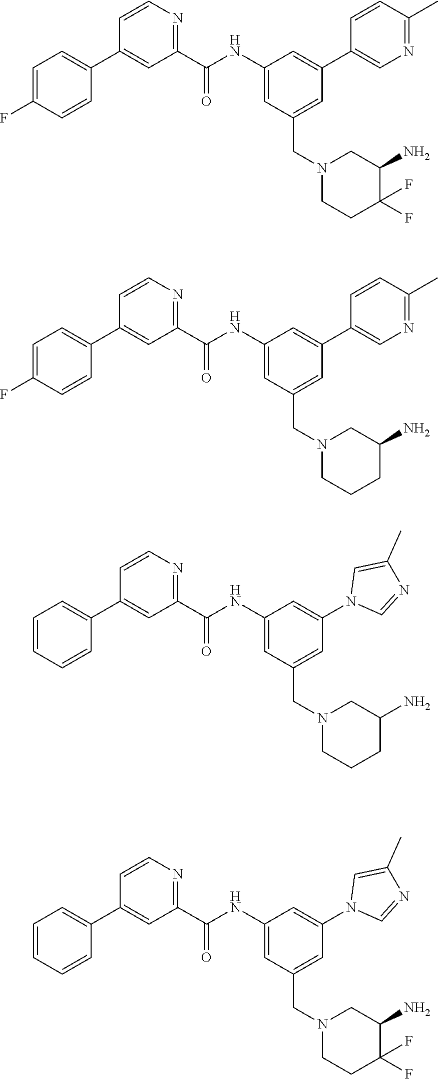 [SMILES: Cc1ccc(-c2cc(CN3CCC(F)(F)[C@H](N)C3)cc(NC(=O)c3cc(-c4ccc(F)cc4)ccn3)c2)cn1 ; Cc1ccc(-c2cc(CN3CCC[C@H](N)C3)cc(NC(=O)c3cc(-c4ccc(F)cc4)ccn3)c2)cn1 ; Cc1cn(-c2cc(CN3CCCC(N)C3)cc(NC(=O)c3cc(-c4ccccc4)ccn3)c2)cn1 ; Cc1cn(-c2cc(CN3CCC(F)(F)[C@H](N)C3)cc(NC(=O)c3cc(-c4ccccc4)ccn3)c2)cn1]
or a pharmaceutically acceptable salt, solvate, prodrug or polymorph thereof.

12. The compound selected from 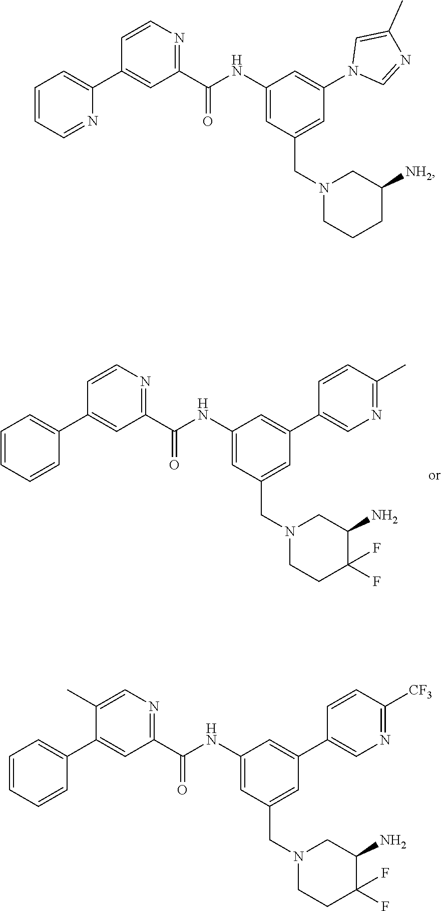 [SMILES: Cc1cn(-c2cc(CN3CCC[C@H](N)C3)cc(NC(=O)c3cc(-c4ccccn4)ccn3)c2)cn1 ; Cc1ccc(-c2cc(CN3CCC(F)(F)[C@H](N)C3)cc(NC(=O)c3cc(-c4ccccc4)ccn3)c2)cn1 ; Cc1cnc(C(=O)Nc2cc(CN3CCC(F)(F)[C@H](N)C3)cc(-c3ccc(C(F)(F)F)nc3)c2)cc1-c1ccccc1]
or a pharmaceutically acceptable salt, solvate, prodrug or polymorph thereof.

13. A composition comprising:
a compound according to claim 1, or a pharmaceutically acceptable salt, solvate, prodrug or polymorph thereof; and
a pharmaceutically acceptable excipient.

14. A composition comprising:
a compound according to claim 1, or a pharmaceutically acceptable salt, solvate, prodrug or polymorph thereof, and
a statin.

15. A method for inhibiting PCSK9 in a subject in need thereof, the method comprising administering a therapeutically effective amount of a compound according to claim 1, or a pharmaceutically acceptable salt, solvate, prodrug or polymorph thereof.

16. A method for reducing LDL in a subject in need thereof, the method comprising administering a therapeutically effective amount of a compound according to claim 1, or a pharmaceutically acceptable salt, solvate, prodrug or polymorph thereof.

17. A method for treating a disease or condition responsive to PCSK9 inhibition in a subject in need thereof, wherein the disease or condition is any one of the following: cardiovascular disease, cerebrovascular disease, atherosclerosis and/or their associated diseases or their symptoms, the method comprising administering a therapeutically effective amount of a compound according to claim 1, or a pharmaceutically acceptable salt, solvate, prodrug or polymorph thereof.

18-23. (canceled)

24. A method according to claim 17, wherein the disease or condition is selected from any one of the following: stroke, heart attack, coronary artery disease and/or hypercholesterolemia.

25. A method of preventing the protein-protein interaction between LDLR and PCSK9, the method comprising administering a compound according to claim 1.

26. A method of increasing LDLR expression of the surface of PCSK9-exposed human lymphocytes the method comprising administering a compound according to claim 1.
